SEQUENCE LISTING (620 sequences)

SEQ ID NO: 1 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 2 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTENQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 3 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTENQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 4 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREKMTKNQVSLTCLVEGFYPSDIAVEWESNGQPENNYKTTPPVLDSDGSFFLYSKLTVDKSRWQQGNVFSCSVMHEALHNHYTQKSLSLSP

SEQ ID NO: 5 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLKSDGSFFLYSELTVDKSRWQQGNVFSCSVMHEALHNHYTQKSLSLSP

SEQ ID NO: 6 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLKSDGSFFLYSDLTVDKSRWQQGNVFSCSVMHEALHNHYTQKSLSLSP

SEQ ID NO: 7 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSELTVDKSRWQQGNVFSCSVMHEALHNHYTQKSLSLSP

SEQ ID NO: 8 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLRSDGSFFLYSDLTVDKSRWQQGNVFSCSVMHEALHNHYTQKSLSLSP

SEQ ID NO: 9 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYETTPPVLKSDGSFFLYSKLTVDKSRWQQGNVFSCSVMHEALHNHYTQKSLSLSP

SEQ ID NO: 10 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLKSDGSFFLYSKLTVDKSRWQQGNVFSCSVMHEALHNHYTQKSLSLSP

SEQ ID NO: 11 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYETTPPVLRSDGSFFLYSKLTVDKSRWQQGNVFSCSVMHEALHNHYTQKSLSLSP

SEQ ID NO: 12 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLRSDGSFFLYSKLTVDKSRWQQGNVFSCSVMHEALHNHYTQKSLSLSP

SEQ ID NO: 13 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTTPPVLCSDGSFFLYSKLTVDKSRWQQGNVFSCSVMHEALHNHYTQKSLSLSP

SEQ ID NO: 14 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPCREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 15 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRCEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 16 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 17 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTCPPCREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTCPPCLDSDGSFFLYSKLTVDKSRWQQGNVFSCSVMHEALHNHYTQKSLSLSP

SEQ ID NO: 19 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 20 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTDPPSREEMTKNQVSLKCLVKGFYPSDIAVEWESNGQPENNYKTTPPVLDSDGSFFLYSKLTVDKSRWQQGNVFSCSVMHEALHNHYTQKSLSLSP

SEQ ID NO: 21 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTEPPSREEMTKNQVSLKCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLDCLVKGFYPSDIAVEWESNGQPENNYKTTPPVLDSDGSFFLYSKLTVDKSRWQQGNVFSCSVMHEALHNHYTQKSLSLSP

SEQ ID NO: 23 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTKPPSREEMTKNQVSLECLVKGFYPSDIAVEWESNGQPENNYKTTPPVLDSDGSFFLYSKLTVDKSRWQQGNVFSCSVMHEALHNHYTQKSLSLSP

SEQ ID NO: 24 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSRLTVDKSRWQQGNVFSCSVMHEALHNHYTQKSLSLSP

SEQ ID NO: 25 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 26 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 27 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTTPPVLDSDGSFFLTSKLTVDKSRWQQGNVFSCSVMHEALHNHYTQKSLSLSP

SEQ ID NO: 28 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLASKLTVDKSRWQQGNVFSCSVMHEALHNHYTQKSLSLSP

SEQ ID NO: 29 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCLVKGFYPSDIAVEWESNGQPENNYKTWPPVLDSDGSFALTSKLTVDKSRWQQGNVFSCSVMHEALHNHYTQKSLSLSP

SEQ ID NO: 30 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRENMTKNQVSLTCLVEGFYPSDIAVEWESNGQPENNYKTTPPVLVSDGSFTLYSWLTVDKSRWQQGNVFSCSVMHEALHNHYTQKSLSLSP

SEQ ID NO: 31 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 32 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTTLPPSREEMTKNQVHLTCLVKGFYPSDIAVEWESNGQPENNYKTFPPVLDSDGSFALYSKLTVDKSRWQQGNVFSCSVMHEALHNHYTQKSLSLSP

SEQ ID NO: 33 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTTLPPSREEMTKNQVHLTCLVKGFYPSDIAVEWESNGQPENNYKTTPPVLDSDGSFFLYSKLTVDKSRWQQGNVFSCSVMHEALHNHYTQKSLSLSP

SEQ ID NO: 34 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFALYSKLTVDKSRWQQGNVFSCSVMHEALHNHYTQKSLSLSP

SEQ ID NO: 35 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREKMTKNQVSLTCLVEGFYPSDIAVEWESNGQPENNYKTTPPVLKSDGSFFLYSELTVDKSRWQQGNVFSCSVMHEALHNHYTQKSLSLSP

SEQ ID NO: 36 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSELTVDKSRWQQGNVFSCSVMHEALHNHYTQESLSLSP

SEQ ID NO: 37 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKKMTKNQVSLTCLVEGFYPSDIAVEWESNGQPENNYKTTPPVLDSDGSFFLYSKLTVDKSRWQQGNVFSCSVMHEALHNHYTQESLSLSP

SEQ ID NO: 38 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKKMTKNQVSLTCLVEGFYPSDIAVEWESNGQPENNYKTTPPVLKSDGSFFLYSELTVDKSRWQQGNVFSCSVMHEALHNHYTQESLSLSP

SEQ ID NO: 39 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPYREEMTKNQVSLYCLVKGFYPSDIAVEWESNGQPENNYKTTPPVLDSDGSFFLTSKLTVDKSRWQQGNVFSCSVMHEALHNHYTQKSLSLSP

SEQ ID NO: 40 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLLCLVKGFYPSDIAVEWESNGQPENNYKTWPPVLDSDGSFALYSKLTVDKSRWQQGNVFSCSVMHEALHNHYTQKSLSLSP

SEQ ID NO: 41 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYLTWPPVLDSDGSFALVSKLTVDKSRWQQGNVFSCSVMHEALHNHYTQKSLSLSP

SEQ ID NO: 42 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVKGFYPSDIAVEWESNGQPENNYCTTPPVLCSDGSFFLYSKLTVDKSRWQQGNVFSCSVMHEALHNHYTQKSLSLSP

SEQ ID NO: 43 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTCPPCRECMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 44 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVKGFYPSDIAVEWESNGQPENNYKTCPPCLDSDGSFFLYSKLTVDKSRWQQGNVFSCSVMHEALHNHYTQKSLSLSP

SEQ ID NO: 45 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVKGFYPSDIAVEWESNGQPENNYCTCPPCLCSDGSFFLYSKLTVDKSRWQQGNVFSCSVMHEALHNHYTQKSLSLSP

SEQ ID NO: 46 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYCTCPPCLCSDGSFFLYSKLTVDKSRWQQGNVFSCSVMHEALHNHYTQKSLSLSP

SEQ ID NO: 47 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTCPPCREEMTKNQVSLTCLVKGFYPSDIAVEWESNGQPENNYCTTPPVLCSDGSFFLYSKLTVDKSRWQQGNVFSCSVMHEALHNHYTQKSLSLSP

SEQ ID NO: 48 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 49 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREKMTKNQVSLTCLVEGFYPSDIAVEWESNGQPENNYDTTPPVLKSDGSFFLYSKLTVDKSRWQQGNVFSCSVMHEALHNHYTQKSLSLSP

SEQ ID NO: 50 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 51 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVKGFYPSDIAVEWESNGQPENNYDTTPPVLKSDGSFFLYSKLTVDKSRWQQGNVFSCSVMHEALHNHYTQKSLSLSP

SEQ ID NO: 52 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREKMTKNQVSLTCLVEGFYPSDIAVEWESNGQPENNYKTCPPCLDSDGSFFLYSKLTVDKSRWQQGNVFSCSVMHEALHNHYTQKSLSLSP

SEQ ID NO: 53 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVEGFYPSDIAVEWESNGQPENNYKTTPPVLDSDGSFFLYSKLTVDKSRWQQGNVFSCSVMHEALHNHYTQKSLSLSP

SEQ ID NO: 54 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREKMTKNQVSLTCLVEGFYPSDIAVEWESNGQPENNYCTTPPVLCSDGSFFLYSKLTVDKSRWQQGNVFSCSVMHEALHNHYTQKSLSLSP

SEQ ID NO: 55 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 56 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 57 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLDSDGSFFLYSKLTVDKSRWQQGNVFSCSVMHEALHNHYTQKSLSLSP

SEQ ID NO: 58 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTENQVSLTCLVKGFYPSDIAVEWESNGQPENNYKTTPPVLMSDGSFFLASVLTVDKSRWQQGNVFSCSVMHEALHNHYTQKSLSLSP

SEQ ID NO: 59 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTCPPCLKSDGSFFLYSKLTVDKSRWQQGNVFSCSVMHEALHNHYTQKSLSLSP

SEQ ID NO: 60 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLLCLVKGFYPSDIAVEWESNGQPENNYLTWPPVLDSDGSFALVSKLTVDKSRWQQGNVFSCSVMHEALHNHYTQKSLSLSP

SEQ ID NO: 61 - protein, 214 aa, Homo sapiens [sequence omitted]

SEQ ID NO: 62 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 63 - protein, 225 aa, Homo sapiens; identical to SEQ ID NO: 62

SEQ ID NO: 64 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 65 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 66 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 67 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFALYSKLTVDKSRWQQGNVFSCSVMHEALHNHYTQKSLSLSP

SEQ ID NO: 68 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSELTVDKSRWQQGNVFSCSVMHEALHNHYTQESLSLSP

SEQ ID NO: 69 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPYREEMTKNQVSLYCLVKGFYPSDIAVEWESNGQPENNYKTTPPVLDSDGSFFLTSKLTVDKSRWQQGNVFSCSVMHEALHNHYTQKSLSLSP

SEQ ID NO: 70 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYLTWPPVLDSDGSFALVSKLTVDKSRWQQGNVFSCSVMHEALHNHYTQKSLSLSP

SEQ ID NO: 71 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 72 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 73 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVKGFYPSDIAVEWESNGQPENNYDTTPPVLKSDGSFFLYSKLTVDKSRWQQGNVFSCSVMHEALHNHYTQKSLSLSP

SEQ ID NO: 74 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 75 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 76 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTENQVSLTCLVKGFYPSDIAVEWESNGQPENNYKTTPPVLMSDGSFFLASVLTVDKSRWQQGNVFSCSVMHEALHNHYTQKSLSLSP

SEQ ID NO: 77 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLLCLVKGFYPSDIAVEWESNGQPENNYLTWPPVLDSDGSFALVSKLTVDKSRWQQGNVFSCSVMHEALHNHYTQKSLSLSP

SEQ ID NO: 78 - protein, 447 aa, Homo sapiens
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 79 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 80 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRESEGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 81 - protein, 447 aa, Homo sapiens
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 82 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 83 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRESEGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSELTVDKSRWQQGNVFSCSVMHEALHNHYTQESLSLSP

SEQ ID NO: 84 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRESEGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 85 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTENQVSLTCLVKGFYPSDIAVEWESNGQPENNYKTTPPVLMSDGSFFLASVLTVDKSRWQQGNVFSCSVMHEALHNHYTQKSLSLSP

SEQ ID NO: 86 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 87 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTWPPMLDSDGSFALYSKLTVDKSRWQQGNVFSCSVMHEALHNHYTQKSLSLSP

SEQ ID NO: 88 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLWCAVKGFYPSDIAVEWESNGQPENNYKTTPPMLDSDGSFFLVSKLTVDKSRWQQGNVFSCSVMHEALHNHYTQKSLSLSP

SEQ ID NO: 89 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPRVYTLPPSREEMTENQVSLTCLVKGFYPSDIAVEWESNGQPENNYKTTPPMLVSDGSFTLYSWLTVDKSRWQQGNVFSCSVMHEALHNHYTQKSLSLSP

SEQ ID NO: 90 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYLTWPPMLDSDGSFALVSKLTVDKSRWQQGNVFSCSVMHEALHNHYTQKSLSLSP

SEQ ID NO: 91 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYDTTPPMLKSDGSFFLYSDLTVDKSRWQQGNVFSCSVMHEALHNHYTQKSLSLSP

SEQ ID NO: 92 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 93 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTWPPVLDSDGSFALYSRLTVDKSRWQEGNVFSCSVMHEALHNHYTQKSLSLSL

SEQ ID NO: 94 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCAVKGFYPSDIAVEWESNGQPENNYKTTPPVLDSDGSFFLVSRLTVDKSRWQEGNVFSCSVMHEALHNHYTQKSLSLSL

SEQ ID NO: 95 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPRVYTLPPSQEEMTENQVSLTCLVKGFYPSDIAVEWESNGQPENNYKTTPPVLVSDGSFTLYSWLTVDKSRWQEGNVFSCSVMHEALHNHYTQKSLSLSL

SEQ ID NO: 96 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYLTWPPVLDSDGSFALVSRLTVDKSRWQEGNVFSCSVMHEALHNHYTQKSLSLSL

SEQ ID NO: 97 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYDTTPPVLKSDGSFFLYSDLTVDKSRWQEGNVFSCSVMHEALHNHYTQKSLSLSL

SEQ ID NO: 98 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSRKEMTKNQVSLTCLVKGFYPSDIAVEWESNGQPENNYKTTPPMLKSDGSFFLYSELTVDKSRWQQGNVFSCSVMHEALHNHYTQKSLSLSP

SEQ ID NO: 99 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPEVYTLPPYREEMTKNQVSLYCLVKGFYPSDIAVEWESNGQPENNYKTTPPMLDSDGSFFLTSKLTVDKSRWQQGNVFSCSVMHEALHNHYTQKSLSLSP

SEQ ID NO: 100 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRRPRVYTLPPSREEMTDNQVSLVCLVKGFYPSDIAVEWESNGQPENNYKTTPPMLMSDGSFFLASVLTVDKSRWQQGNVFSCSVMHEALHNHYTQKSLSLSP

SEQ ID NO: 101 - protein, 494 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSP

SEQ ID NO: 102 - protein, 227 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 103 - protein, 226 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSP

SEQ ID NO: 104 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSRKEMTKNQVSLTCLVKGFYPSDIAVEWESNGQPENNYDTTPPMLKSDGSFFLYSKLTVDKSRWQQGNVFSCSVMHEALHNHYTQESLSLSP

SEQ ID NO: 105 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSRKEMTKNQVSLTCLVKGFYPSDIAVEWESNGQPENNYDTTPPMLKSDGSFFLYSDLTVDKSRWQQGNVFSCSVMHEALHNHYTQKSLSLSP

SEQ ID NO: 106 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPRRPQVYTLPPSREEMTENQVSLTCLVKGFYPSDIAVEWESNGQPENNYKTTPPMLMSDGSFFLASVLTVDKSRWQQGNVFSCSVMHEALHNHYTQKSLSLSP

SEQ ID NO: 107 - protein, 443 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYVYPPSREEMTKNQVSLLCLVKGFYPSDIAVEWESNGQPENNYLTWPPMLDSDGSFALVSKLTVDKSRWQQGNVFSCSVMHEALHNHYTQKSLSLSP

SEQ ID NO: 108 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYDTTPPVLKSDGSFFLYSRLTVDKSRWQEGNVFSCSVMHEALHNHYTQESLSLSL

SEQ ID NO: 109 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLKSDGSFFLYSELTVDKSRWQEGNVFSCSVMHEALHNHYTQESLSLSL

SEQ ID NO: 110 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPEVYTLPPYQEEMTKNQVSLYCLVKGFYPSDIAVEWESNGQPENNYKTTPPVLDSDGSFFLTSRLTVDKSRWQEGNVFSCSVMHEALHNHYTQKSLSLSL

SEQ ID NO: 111 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPRRPRVYTLPPSQEEMTDNQVSLVCLVKGFYPSDIAVEWESNGQPENNYKTTPPVLMSDGSFFLASVLTVDKSRWQEGNVFSCSVMHEALHNHYTQKSLSLSL

SEQ ID NO: 112 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYDTTPPVLKSDGSFFLYSDLTVDKSRWQEGNVFSCSVMHEALHNHYTQKSLSLSL

SEQ ID NO: 113 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPRRPQVYTLPPSQEEMTENQVSLTCLVKGFYPSDIAVEWESNGQPENNYKTTPPVLMSDGSFFLASVLTVDKSRWQEGNVFSCSVMHEALHNHYTQKSLSLSL

SEQ ID NO: 114 - protein, 444 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYVYPPSQEEMTKNQVSLLCLVKGFYPSDIAVEWESNGQPENNYLTWPPVLDSDGSFALVSRLTVDKSRWQEGNVFSCSVMHEALHNHYTQKSLSLSL

SEQ ID NO: 115 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 116 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 117 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 118 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 119 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSELTVDKSRWQQGNVFSCSVMHEALHNHYTQESLSLSP

SEQ ID NO: 120 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 121 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 122 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPRRPQVYTLPPSREEMTENQVSLTCLVKGFYPSDIAVEWESNGQPENNYKTTPPVLMSDGSFFLASVLTVDKSRWQQGNVFSCSVMHEALHNHYTQKSLSLSP

SEQ ID NO: 123 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYVYPPSREEMTKNQVSLLCLVKGFYPSDIAVEWESNGQPENNYLTWPPVLDSDGSFALVSKLTVDKSRWQQGNVFSCSVMHEALHNHYTQKSLSLSP

SEQ ID NO: 124 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHTTRKELSLSP

SEQ ID NO: 125 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVLHEALHAHTTRKELSLSP

SEQ ID NO: 126 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVLHEALHAHTTRKELSLSP

SEQ ID NO: 127 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVLHEALHAHTTRKELSLSP

SEQ ID NO: 128 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSELTVDKSRWQQGNVFSCSVLHEALHAHTTREELSLSP

SEQ ID NO: 129 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVLHEALHAHTTREELSLSP

SEQ ID NO: 130 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLKSDGSFFLYSDLTVDKSRWQQGNVFSCSVLHEALHAHTTRKELSLSP

SEQ ID NO: 131 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTENQVSLTCLVKGFYPSDIAVEWESNGQPENNYKTTPPVLMSDGSFFLASVLTVDKSRWQQGNVFSCSVLHEALHAHTTRKELSLSP

SEQ ID NO: 132 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLLCLVKGFYPSDIAVEWESNGQPENNYLTWPPVLDSDGSFALVSKLTVDKSRWQQGNVFSCSVLHEALHAHTTRKELSLSP

SEQ ID NO: 133 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 134 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 135 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 136 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 137 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 138 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 139 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 140 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 141 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 142 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 143 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 144 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 145 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 146 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 147 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 148 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSP

SEQ ID NO: 149 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVLHEALHSHYTQKSLSLSP

SEQ ID NO: 150 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVLHEALHSHYTQKSLSLSP

SEQ ID NO: 151 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVLHEALHSHYTQKSLSLSP

SEQ ID NO: 152 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVLHEALHSHYTQESLSLSP

SEQ ID NO: 153 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 154 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 155 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 156 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 157 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 158 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 159 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 160 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 161 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSP

SEQ ID NO: 162 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTWPPVLDSDGSFALYSKLTVDKSRWQQGNVFSCSVMHGALHNHYTQKYLSLSP

SEQ ID NO: 163 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLWCAVKGFYPSDIAVEWESNGQPENNYKTTPPVLDSDGSFFLVSKLTVDKSRWQQGNVFSCSVMHGALHNHYTQKYLSLSP

SEQ ID NO: 164 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTENQVSLTCLVKGFYPSDIAVEWESNGQPENNYKTTPPVLVSDGSFTLYSWLTVDKSRWQQGNVFSCSVMHGALHNHYTQKYLSLSP

SEQ ID NO: 165 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKYLSLSP

SEQ ID NO: 166 - protein, 466 aa, Homo sapiens
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKGGGGSGGGGSGGGGSQVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 167 - protein, 466 aa, Homo sapiens
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKGGGGSGGGGSGGGGSQVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 168 - protein, 466 aa, Homo sapiens
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKGGGGSGGGGSGGGGSQVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 169 - protein, 466 aa, Homo sapiens
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKGGGGSGGGGSGGGGSQVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 170 - protein, 466 aa, Homo sapiens
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKGGGGSGGGGSGGGGSQVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 171 - protein, 466 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 172 - protein, 466 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 173 - protein, 466 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 174 - protein, 466 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 175 - protein, 466 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 176 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRREMTKNQVSLTCLVKGFYPSDIAVEWESNGQPENNYKTTPPVLKSDGSFFLYSELTVDKSRWQQGNVFSCSVMHEALHNHYTQESLSLSP

SEQ ID NO: 177 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRHEMTKNQVSLTCLVKGFYPSDIAVEWESNGQPENNYKTTPPVLKSDGSFFLYSELTVDKSRWQQGNVFSCSVMHEALHNHYTQESLSLSP

SEQ ID NO: 178 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLRSDGSFFLYSELTVDKSRWQQGNVFSCSVMHEALHNHYTQESLSLSP

SEQ ID NO: 179 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLHSDGSFFLYSELTVDKSRWQQGNVFSCSVMHEALHNHYTQESLSLSP

SEQ ID NO: 180 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSDLTVDKSRWQQGNVFSCSVMHEALHNHYTQESLSLSP

SEQ ID NO: 181 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSELTVDKSRWQQGNVFSCSVMHEALHNHYTQDSLSLSP

SEQ ID NO: 182 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLRSDGSFFLYSDLTVDKSRWQQGNVFSCSVMHEALHNHYTQESLSLSP

SEQ ID NO: 183 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLHSDGSFFLYSDLTVDKSRWQQGNVFSCSVMHEALHNHYTQESLSLSP

SEQ ID NO: 184 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRREMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 185 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQDSLSLSP

SEQ ID NO: 186 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLRSDGSFFLYSKLTVDKSRWQQGNVFSCSVMHEALHNHYTQESLSLSP

SEQ ID NO: 187 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYETTPPVLKSDGSFFLYSKLTVDKSRWQQGNVFSCSVMHEALHNHYTQESLSLSP

SEQ ID NO: 188 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLRSDGSFFLYSDLTVDKSRWQQGNVFSCSVMHEALHNHYTQKSLSLSP

SEQ ID NO: 189 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLKSDGSFFLYSELTVDKSRWQQGNVFSCSVMHEALHNHYTQKSLSLSP

SEQ ID NO: 190 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYETTPPVLKSDGSFFLYSDLTVDKSRWQQGNVFSCSVMHEALHNHYTQKSLSLSP

SEQ ID NO: 191 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSELTVDKSRWQQGNVFSCSVMHEALHNHYTQKSLSLSP

SEQ ID NO: 192 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLRSDGSFFLYSDLTVDKSRWQQGNVFSCSVMHEALHNHYTQKSLSLSP

SEQ ID NO: 193 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRREMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 194 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRHEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 195 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYETTPPVLKSDGSFFLYSDLTVDKSRWQQGNVFSCSVMHEALHNHYTQKSLSLSP

SEQ ID NO: 196 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRREMTKNQVSLTCLVKGFYPSDIAVEWESNGQPENNYDTTPPVLKSDGSFFLYSKLTVDKSRWQQGNVFSCSVMHEALHNHYTQDSLSLSP

SEQ ID NO: 197 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLHSDGSFFLYSDLTVDKSRWQQGNVFSCSVMHEALHNHYTQKSLSLSP

SEQ ID NO: 198 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRREMTKNQVSLTCLVKGFYPSDIAVEWESNGQPENNYKTTPPVLKSDGSFFLYSELTVDKSRWQQGNVFSCSVMHEALHNHYTQDSLSLSP

SEQ ID NO: 199 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRHEMTKNQVSLTCLVKGFYPSDIAVEWESNGQPENNYKTTPPVLKSDGSFFLYSELTVDKSRWQQGNVFSCSVMHEALHNHYTQDSLSLSP

SEQ ID NO: 200 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTDNQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 201 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPKVYTLPPSREEMTDNQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 202 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTENQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 203 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPKVYTLPPSREEMTDNQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 204 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 205 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPKVYTLPPSREEMTENQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 206 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASQLTVDKSRWQQGNVFSCSVMHEALHNHYTQKSLSLSP

SEQ ID NO: 207 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASQLTVDKSRWQQGNVFSCSVMHEALHNHYTQKSLSLSP

SEQ ID NO: 208 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASQLTVDKSRWQQGNVFSCSVMHEALHNHYTQKSLSLSP

SEQ ID NO: 209 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASQLTVDKSRWQQGNVFSCSVMHEALHNHYTQKSLSLSP

SEQ ID NO: 210 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASQLTVDKSRWQQGNVFSCSVMHEALHNHYTQKSLSLSP

SEQ ID NO: 211 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASQLTVDKSRWQQGNVFSCSVMHEALHNHYTQKSLSLSP

SEQ ID NO: 212 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASQLTVDKSRWQQGNVFSCSVMHEALHNHYTQKSLSLSP

SEQ ID NO: 213 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASQLTVDKSRWQQGNVFSCSVMHEALHNHYTQKSLSLSP

SEQ ID NO: 214 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASNLTVDKSRWQQGNVFSCSVMHEALHNHYTQKSLSLSP

SEQ ID NO: 215 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASNLTVDKSRWQQGNVFSCSVMHEALHNHYTQKSLSLSP

SEQ ID NO: 216 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASNLTVDKSRWQQGNVFSCSVMHEALHNHYTQKSLSLSP

SEQ ID NO: 217 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASNLTVDKSRWQQGNVFSCSVMHEALHNHYTQKSLSLSP

SEQ ID NO: 218 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASNLTVDKSRWQQGNVFSCSVMHEALHNHYTQKSLSLSP

SEQ ID NO: 219 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASNLTVDKSRWQQGNVFSCSVMHEALHNHYTQKSLSLSP

SEQ ID NO: 220 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASNLTVDKSRWQQGNVFSCSVMHEALHNHYTQKSLSLSP

SEQ ID NO: 221 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASNLTVDKSRWQQGNVFSCSVMHEALHNHYTQKSLSLSP

SEQ ID NO: 222 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASHLTVDKSRWQQGNVFSCSVMHEALHNHYTQKSLSLSP

SEQ ID NO: 223 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASHLTVDKSRWQQGNVFSCSVMHEALHNHYTQKSLSLSP

SEQ ID NO: 224 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASHLTVDKSRWQQGNVFSCSVMHEALHNHYTQKSLSLSP

SEQ ID NO: 225 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASHLTVDKSRWQQGNVFSCSVMHEALHNHYTQKSLSLSP

SEQ ID NO: 226 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASHLTVDKSRWQQGNVFSCSVMHEALHNHYTQKSLSLSP

SEQ ID NO: 227 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASHLTVDKSRWQQGNVFSCSVMHEALHNHYTQKSLSLSP

SEQ ID NO: 228 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASHLTVDKSRWQQGNVFSCSVMHEALHNHYTQKSLSLSP

SEQ ID NO: 229 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASHLTVDKSRWQQGNVFSCSVMHEALHNHYTQKSLSLSP

SEQ ID NO: 230 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASHLTVDKSRWQQGNVFSCSVMHEALHNHYTQKSLSLSP

SEQ ID NO: 231 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASVLTVDKSRWQQGNVFSCSVMHEALHNHYTQKSLSLSP

SEQ ID NO: 232 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASVLTVDKSRWQQGNVFSCSVMHEALHNHYTQKSLSLSP

SEQ ID NO: 233 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASVLTVDKSRWQQGNVFSCSVMHEALHNHYTQKSLSLSP

SEQ ID NO: 234 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASVLTVDKSRWQQGNVFSCSVMHEALHNHYTQKSLSLSP

SEQ ID NO: 235 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASVLTVDKSRWQQGNVFSCSVMHEALHNHYTQKSLSLSP

SEQ ID NO: 236 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASVLTVDKSRWQQGNVFSCSVMHEALHNHYTQKSLSLSP

SEQ ID NO: 237 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASVLTVDKSRWQQGNVFSCSVMHEALHNHYTQKSLSLSP

SEQ ID NO: 238 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASVLTVDKSRWQQGNVFSCSVMHEALHNHYTQKSLSLSP

SEQ ID NO: 239 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASVLTVDKSRWQQGNVFSCSVMHEALHNHYTQKSLSLSP

SEQ ID NO: 240 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASVLTVDKSRWQQGNVFSCSVMHEALHNHYTQKSLSLSP

SEQ ID NO: 241 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASLLTVDKSRWQQGNVFSCSVMHEALHNHYTQKSLSLSP

SEQ ID NO: 242 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASLLTVDKSRWQQGNVFSCSVMHEALHNHYTQKSLSLSP

SEQ ID NO: 243 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASLLTVDKSRWQQGNVFSCSVMHEALHNHYTQKSLSLSP

SEQ ID NO: 244 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASLLTVDKSRWQQGNVFSCSVMHEALHNHYTQKSLSLSP

SEQ ID NO: 245 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASLLTVDKSRWQQGNVFSCSVMHEALHNHYTQKSLSLSP

SEQ ID NO: 246 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASLLTVDKSRWQQGNVFSCSVMHEALHNHYTQKSLSLSP

SEQ ID NO: 247 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASLLTVDKSRWQQGNVFSCSVMHEALHNHYTQKSLSLSP

SEQ ID NO: 248 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASILTVDKSRWQQGNVFSCSVMHEALHNHYTQKSLSLSP

SEQ ID NO: 249 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASILTVDKSRWQQGNVFSCSVMHEALHNHYTQKSLSLSP

SEQ ID NO: 250 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASILTVDKSRWQQGNVFSCSVMHEALHNHYTQKSLSLSP

SEQ ID NO: 251 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASILTVDKSRWQQGNVFSCSVMHEALHNHYTQKSLSLSP

SEQ ID NO: 252 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASILTVDKSRWQQGNVFSCSVMHEALHNHYTQKSLSLSP

SEQ ID NO: 253 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASILTVDKSRWQQGNVFSCSVMHEALHNHYTQKSLSLSP

SEQ ID NO: 254 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASILTVDKSRWQQGNVFSCSVMHEALHNHYTQKSLSLSP

SEQ ID NO: 255 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASILTVDKSRWQQGNVFSCSVMHEALHNHYTQKSLSLSP

SEQ ID NO: 256 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASILTVDKSRWQQGNVFSCSVMHEALHNHYTQKSLSLSP

SEQ ID NO: 257 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASFLTVDKSRWQQGNVFSCSVMHEALHNHYTQKSLSLSP

SEQ ID NO: 258 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASFLTVDKSRWQQGNVFSCSVMHEALHNHYTQKSLSLSP

SEQ ID NO: 259 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASFLTVDKSRWQQGNVFSCSVMHEALHNHYTQKSLSLSP

SEQ ID NO: 260 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASFLTVDKSRWQQGNVFSCSVMHEALHNHYTQKSLSLSP

SEQ ID NO: 261 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASFLTVDKSRWQQGNVFSCSVMHEALHNHYTQKSLSLSP

SEQ ID NO: 262 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASFLTVDKSRWQQGNVFSCSVMHEALHNHYTQKSLSLSP

SEQ ID NO: 263 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASFLTVDKSRWQQGNVFSCSVMHEALHNHYTQKSLSLSP

SEQ ID NO: 264 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASFLTVDKSRWQQGNVFSCSVMHEALHNHYTQKSLSLSP

SEQ ID NO: 265 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASFLTVDKSRWQQGNVFSCSVMHEALHNHYTQKSLSLSP

SEQ ID NO: 266 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASYLTVDKSRWQQGNVFSCSVMHEALHNHYTQKSLSLSP

SEQ ID NO: 267 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASYLTVDKSRWQQGNVFSCSVMHEALHNHYTQKSLSLSP

SEQ ID NO: 268 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASYLTVDKSRWQQGNVFSCSVMHEALHNHYTQKSLSLSP

SEQ ID NO: 269 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASYLTVDKSRWQQGNVFSCSVMHEALHNHYTQKSLSLSP

SEQ ID NO: 270 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASYLTVDKSRWQQGNVFSCSVMHEALHNHYTQKSLSLSP

SEQ ID NO: 271 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASYLTVDKSRWQQGNVFSCSVMHEALHNHYTQKSLSLSP

SEQ ID NO: 272 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASYLTVDKSRWQQGNVFSCSVMHEALHNHYTQKSLSLSP

SEQ ID NO: 273 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASYLTVDKSRWQQGNVFSCSVMHEALHNHYTQKSLSLSP

SEQ ID NO: 274 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASTLTVDKSRWQQGNVFSCSVMHEALHNHYTQKSLSLSP

SEQ ID NO: 275 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASTLTVDKSRWQQGNVFSCSVMHEALHNHYTQKSLSLSP

SEQ ID NO: 276 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASTLTVDKSRWQQGNVFSCSVMHEALHNHYTQKSLSLSP

SEQ ID NO: 277 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASTLTVDKSRWQQGNVFSCSVMHEALHNHYTQKSLSLSP

SEQ ID NO: 278 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASTLTVDKSRWQQGNVFSCSVMHEALHNHYTQKSLSLSP

SEQ ID NO: 279 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASTLTVDKSRWQQGNVFSCSVMHEALHNHYTQKSLSLSP

SEQ ID NO: 280 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASTLTVDKSRWQQGNVFSCSVMHEALHNHYTQKSLSLSP

SEQ ID NO: 281 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASTLTVDKSRWQQGNVFSCSVMHEALHNHYTQKSLSLSP

SEQ ID NO: 282 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASTLTVDKSRWQQGNVFSCSVMHEALHNHYTQKSLSLSP

SEQ ID NO: 283 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASSLTVDKSRWQQGNVFSCSVMHEALHNHYTQKSLSLSP

SEQ ID NO: 284 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASSLTVDKSRWQQGNVFSCSVMHEALHNHYTQKSLSLSP

SEQ ID NO: 285 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLHSDGSFFLASSLTVDKSRWQQGNVFSCSVMHEALHNHYTQKSLSLSP

SEQ ID NO: 286 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASSLTVDKSRWQQGNVFSCSVMHEALHNHYTQKSLSLSP

SEQ ID NO: 287 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASSLTVDKSRWQQGNVFSCSVMHEALHNHYTQKSLSLSP

SEQ ID NO: 288 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASSLTVDKSRWQQGNVFSCSVMHEALHNHYTQKSLSLSP

SEQ ID NO: 289 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLFSDGSFFLASSLTVDKSRWQQGNVFSCSVMHEALHNHYTQKSLSLSP

SEQ ID NO: 290 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASSLTVDKSRWQQGNVFSCSVMHEALHNHYTQKSLSLSP

SEQ ID NO: 291 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASSLTVDKSRWQQGNVFSCSVMHEALHNHYTQKSLSLSP

SEQ ID NO: 292 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASSLTVDKSRWQQGNVFSCSVMHEALHNHYTQKSLSLSP

SEQ ID NO: 293 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASQLTVDKSRWQQGNVFSCSVMHEALHNHYTQKSLSLSP

SEQ ID NO: 294 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASNLTVDKSRWQQGNVFSCSVMHEALHNHYTQKSLSLSP

SEQ ID NO: 295 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASHLTVDKSRWQQGNVFSCSVMHEALHNHYTQKSLSLSP

SEQ ID NO: 296 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASLLTVDKSRWQQGNVFSCSVMHEALHNHYTQKSLSLSP

SEQ ID NO: 297 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASILTVDKSRWQQGNVFSCSVMHEALHNHYTQKSLSLSP

SEQ ID NO: 298 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASFLTVDKSRWQQGNVFSCSVMHEALHNHYTQKSLSLSP

SEQ ID NO: 299 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASYLTVDKSRWQQGNVFSCSVMHEALHNHYTQKSLSLSP

SEQ ID NO: 300 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASTLTVDKSRWQQGNVFSCSVMHEALHNHYTQKSLSLSP

SEQ ID NO: 301 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASSLTVDKSRWQQGNVFSCSVMHEALHNHYTQKSLSLSP

SEQ ID NO: 302 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASQLTVDKSRWQQGNVFSCSVMHEALHNHYTQKSLSLSP

SEQ ID NO: 303 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASQLTVDKSRWQQGNVFSCSVMHEALHNHYTQKSLSLSP

SEQ ID NO: 304 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASNLTVDKSRWQQGNVFSCSVMHEALHNHYTQKSLSLSP

SEQ ID NO: 305 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASHLTVDKSRWQQGNVFSCSVMHEALHNHYTQKSLSLSP

SEQ ID NO: 306 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLVSDGSFFLASLLTVDKSRWQQGNVFSCSVMHEALHNHYTQKSLSLSP

SEQ ID NO: 307 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLYSDGSFFLASLLTVDKSRWQQGNVFSCSVMHEALHNHYTQKSLSLSP

SEQ ID NO: 308 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLSSDGSFFLASFLTVDKSRWQQGNVFSCSVMHEALHNHYTQKSLSLSP

SEQ ID NO: 309 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASYLTVDKSRWQQGNVFSCSVMHEALHNHYTQKSLSLSP

SEQ ID NO: 310 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLTSDGSFFLASYLTVDKSRWQQGNVFSCSVMHEALHNHYTQKSLSLSP

SEQ ID NO: 311 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLQSDGSFFLASTLTVDKSRWQQGNVFSCSVMHEALHNHYTQKSLSLSP

SEQ ID NO: 312 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLNSDGSFFLASNLTVDKSRWQQGNVFSCSVMHEALHNHYTQKSLSLSP

SEQ ID NO: 313 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSFLTVDKSRWQQGNVFSCSVMHEALHNHYTQKSLSLSP

SEQ ID NO: 314 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPKVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 315 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPYVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 316 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTDNQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 317 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPKVYTLPPSREEMTDNQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 318 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPHVYTLPPSREEMTDNQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 319 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPYVYTLPPSREEMTDNQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 320 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLLSDGSFTLYSWLTVDKSRWQQGNVFSCSVMHEALHNHYTQKSLSLSP

SEQ ID NO: 321 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLISDGSFTLYSWLTVDKSRWQQGNVFSCSVMHEALHNHYTQKSLSLSP

SEQ ID NO: 322 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLMSDGSFTLYSWLTVDKSRWQQGNVFSCSVMHEALHNHYTQKSLSLSP

SEQ ID NO: 323 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLSSDGSFTLYSWLTVDKSRWQQGNVFSCSVMHEALHNHYTQKSLSLSP

SEQ ID NO: 324 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLTSDGSFTLYSWLTVDKSRWQQGNVFSCSVMHEALHNHYTQKSLSLSP

SEQ ID NO: 325 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLCSDGSFTLYSWLTVDKSRWQQGNVFSCSVMHEALHNHYTQKSLSLSP

SEQ ID NO: 326 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLHSDGSFTLYSWLTVDKSRWQQGNVFSCSVMHEALHNHYTQKSLSLSP

SEQ ID NO: 327 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFALYSWLTVDKSRWQQGNVFSCSVMHEALHNHYTQKSLSLSP

SEQ ID NO: 328 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFVLYSWLTVDKSRWQQGNVFSCSVMHEALHNHYTQKSLSLSP

SEQ ID NO: 329 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFSLYSWLTVDKSRWQQGNVFSCSVMHEALHNHYTQKSLSLSP

SEQ ID NO: 330 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFCLYSWLTVDKSRWQQGNVFSCSVMHEALHNHYTQKSLSLSP

SEQ ID NO: 331 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFNLYSWLTVDKSRWQQGNVFSCSVMHEALHNHYTQKSLSLSP

SEQ ID NO: 332 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFDLYSWLTVDKSRWQQGNVFSCSVMHEALHNHYTQKSLSLSP

SEQ ID NO: 333 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSYLTVDKSRWQQGNVFSCSVMHEALHNHYTQKSLSLSP

SEQ ID NO: 334 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSHLTVDKSRWQQGNVFSCSVMHEALHNHYTQKSLSLSP

SEQ ID NO: 335 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLASDGSFTLYSFLTVDKSRWQQGNVFSCSVMHEALHNHYTQKSLSLSP

SEQ ID NO: 336 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLLSDGSFTLYSFLTVDKSRWQQGNVFSCSVMHEALHNHYTQKSLSLSP

SEQ ID NO: 337 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLISDGSFTLYSFLTVDKSRWQQGNVFSCSVMHEALHNHYTQKSLSLSP

SEQ ID NO: 338 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLMSDGSFTLYSFLTVDKSRWQQGNVFSCSVMHEALHNHYTQKSLSLSP

SEQ ID NO: 339 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLTSDGSFTLYSFLTVDKSRWQQGNVFSCSVMHEALHNHYTQKSLSLSP

SEQ ID NO: 340 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLCSDGSFTLYSFLTVDKSRWQQGNVFSCSVMHEALHNHYTQKSLSLSP

SEQ ID NO: 341 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLNSDGSFTLYSFLTVDKSRWQQGNVFSCSVMHEALHNHYTQKSLSLSP

SEQ ID NO: 342 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLQSDGSFTLYSFLTVDKSRWQQGNVFSCSVMHEALHNHYTQKSLSLSP

SEQ ID NO: 343 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLHSDGSFTLYSFLTVDKSRWQQGNVFSCSVMHEALHNHYTQKSLSLSP

SEQ ID NO: 344 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLASDGSFTLYSYLTVDKSRWQQGNVFSCSVMHEALHNHYTQKSLSLSP

SEQ ID NO: 345 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLLSDGSFTLYSYLTVDKSRWQQGNVFSCSVMHEALHNHYTQKSLSLSP

SEQ ID NO: 346 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLMSDGSFTLYSYLTVDKSRWQQGNVFSCSVMHEALHNHYTQKSLSLSP

SEQ ID NO: 347 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLSSDGSFTLYSYLTVDKSRWQQGNVFSCSVMHEALHNHYTQKSLSLSP

SEQ ID NO: 348 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLTSDGSFTLYSYLTVDKSRWQQGNVFSCSVMHEALHNHYTQKSLSLSP

SEQ ID NO: 349 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLCSDGSFTLYSYLTVDKSRWQQGNVFSCSVMHEALHNHYTQKSLSLSP

SEQ ID NO: 350 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLNSDGSFTLYSYLTVDKSRWQQGNVFSCSVMHEALHNHYTQKSLSLSP

SEQ ID NO: 351 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLQSDGSFTLYSYLTVDKSRWQQGNVFSCSVMHEALHNHYTQKSLSLSP

SEQ ID NO: 352 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLHSDGSFTLYSYLTVDKSRWQQGNVFSCSVMHEALHNHYTQKSLSLSP

SEQ ID NO: 353 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFALYSFLTVDKSRWQQGNVFSCSVMHEALHNHYTQKSLSLSP

SEQ ID NO: 354 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFVLYSFLTVDKSRWQQGNVFSCSVMHEALHNHYTQKSLSLSP

SEQ ID NO: 355 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFSLYSFLTVDKSRWQQGNVFSCSVMHEALHNHYTQKSLSLSP

SEQ ID NO: 356 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFCLYSFLTVDKSRWQQGNVFSCSVMHEALHNHYTQKSLSLSP

SEQ ID NO: 357 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFDLYSFLTVDKSRWQQGNVFSCSVMHEALHNHYTQKSLSLSP

SEQ ID NO: 358 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFALYSYLTVDKSRWQQGNVFSCSVMHEALHNHYTQKSLSLSP

SEQ ID NO: 359 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFCLYSYLTVDKSRWQQGNVFSCSVMHEALHNHYTQKSLSLSP

SEQ ID NO: 360 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFNLYSYLTVDKSRWQQGNVFSCSVMHEALHNHYTQKSLSLSP

SEQ ID NO: 361 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFDLYSYLTVDKSRWQQGNVFSCSVMHEALHNHYTQKSLSLSP

SEQ ID NO: 362 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLLSDGSFALYSWLTVDKSRWQQGNVFSCSVMHEALHNHYTQKSLSLSP

SEQ ID NO: 363 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLLSDGSFSLYSWLTVDKSRWQQGNVFSCSVMHEALHNHYTQKSLSLSP

SEQ ID NO: 364 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLLSDGSFCLYSWLTVDKSRWQQGNVFSCSVMHEALHNHYTQKSLSLSP

SEQ ID NO: 365 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLISDGSFSLYSWLTVDKSRWQQGNVFSCSVMHEALHNHYTQKSLSLSP

SEQ ID NO: 366 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLISDGSFCLYSWLTVDKSRWQQGNVFSCSVMHEALHNHYTQKSLSLSP

SEQ ID NO: 367 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLMSDGSFALYSWLTVDKSRWQQGNVFSCSVMHEALHNHYTQKSLSLSP

SEQ ID NO: 368 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLMSDGSFSLYSWLTVDKSRWQQGNVFSCSVMHEALHNHYTQKSLSLSP

SEQ ID NO: 369 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLMSDGSFCLYSWLTVDKSRWQQGNVFSCSVMHEALHNHYTQKSLSLSP

SEQ ID NO: 370 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLGSDGSFTLYSWLTVDKSRWQQGNVFSCSVMHEALHNHYTQKSLSLSP

SEQ ID NO: 371 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPHVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 372 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLASDGSFTLYSWLTVDKSRWQQGNVFSCSVMHEALHNHYTQKSLSLSP

SEQ ID NO: 373 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLNSDGSFTLYSWLTVDKSRWQQGNVFSCSVMHEALHNHYTQKSLSLSP

SEQ ID NO: 374 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLQSDGSFTLYSWLTVDKSRWQQGNVFSCSVMHEALHNHYTQKSLSLSP

SEQ ID NO: 375 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLSSDGSFTLYSFLTVDKSRWQQGNVFSCSVMHEALHNHYTQKSLSLSP

SEQ ID NO: 376 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFNLYSFLTVDKSRWQQGNVFSCSVMHEALHNHYTQKSLSLSP

SEQ ID NO: 377 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFVLYSYLTVDKSRWQQGNVFSCSVMHEALHNHYTQKSLSLSP

SEQ ID NO: 378 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFSLYSYLTVDKSRWQQGNVFSCSVMHEALHNHYTQKSLSLSP

SEQ ID NO: 379 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLISDGSFALYSWLTVDKSRWQQGNVFSCSVMHEALHNHYTQKSLSLSP

SEQ ID NO: 380 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFGLYSWLTVDKSRWQQGNVFSCSVMHEALHNHYTQKSLSLSP

SEQ ID NO: 381 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFSLYSKLTVDKSRWQQGNVFSCSVMHEALHNHYTQKSLSLSP

SEQ ID NO: 382 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFTLYSKLTVDKSRWQQGNVFSCSVMHEALHNHYTQKSLSLSP

SEQ ID NO: 383 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTYPPVLDSDGSFALYSKLTVDKSRWQQGNVFSCSVMHEALHNHYTQKSLSLSP

SEQ ID NO: 384 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTHPPVLDSDGSFALYSKLTVDKSRWQQGNVFSCSVMHEALHNHYTQKSLSLSP

SEQ ID NO: 385 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTYPPVLDSDGSFTLYSKLTVDKSRWQQGNVFSCSVMHEALHNHYTQKSLSLSP

SEQ ID NO: 386 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTYPPVLDSDGSFCLYSKLTVDKSRWQQGNVFSCSVMHEALHNHYTQKSLSLSP

SEQ ID NO: 387 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTYPPVLDSDGSFVLYSKLTVDKSRWQQGNVFSCSVMHEALHNHYTQKSLSLSP

SEQ ID NO: 388 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFGLYSKLTVDKSRWQQGNVFSCSVMHEALHNHYTQKSLSLSP

SEQ ID NO: 389 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFCLYSKLTVDKSRWQQGNVFSCSVMHEALHNHYTQKSLSLSP

SEQ ID NO: 390 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFVLYSKLTVDKSRWQQGNVFSCSVMHEALHNHYTQKSLSLSP

SEQ ID NO: 391 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTHPPVLDSDGSFGLYSKLTVDKSRWQQGNVFSCSVMHEALHNHYTQKSLSLSP

SEQ ID NO: 392 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTHPPVLDSDGSFSLYSKLTVDKSRWQQGNVFSCSVMHEALHNHYTQKSLSLSP

SEQ ID NO: 393 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTHPPVLDSDGSFTLYSKLTVDKSRWQQGNVFSCSVMHEALHNHYTQKSLSLSP

SEQ ID NO: 394 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTHPPVLDSDGSFCLYSKLTVDKSRWQQGNVFSCSVMHEALHNHYTQKSLSLSP

SEQ ID NO: 395 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTCPPVLDSDGSFCLYSKLTVDKSRWQQGNVFSCSVMHEALHNHYTQKSLSLSP

SEQ ID NO: 396 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFCLYSKLTVDKSRWQQGNVFSCSVMHEALHNHYTQKSLSLSP

SEQ ID NO: 397 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFVLYSKLTVDKSRWQQGNVFSCSVMHEALHNHYTQKSLSLSP

SEQ ID NO: 398 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTYPPVLDSDGSFGLYSKLTVDKSRWQQGNVFSCSVMHEALHNHYTQKSLSLSP

SEQ ID NO: 399 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTYPPVLDSDGSFSLYSKLTVDKSRWQQGNVFSCSVMHEALHNHYTQKSLSLSP

SEQ ID NO: 400 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFSLYSKLTVDKSRWQQGNVFSCSVMHEALHNHYTQKSLSLSP

SEQ ID NO: 401 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFTLYSKLTVDKSRWQQGNVFSCSVMHEALHNHYTQKSLSLSP

SEQ ID NO: 402 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTHPPVLDSDGSFVLYSKLTVDKSRWQQGNVFSCSVMHEALHNHYTQKSLSLSP

SEQ ID NO: 403 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFGLYSKLTVDKSRWQQGNVFSCSVMHEALHNHYTQKSLSLSP

SEQ ID NO: 404 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLVSKLTVDKSRWQQGNVFSCSVMHEALHNHYTQKSLSLSP

SEQ ID NO: 405 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLVSKLTVDKSRWQQGNVFSCSVMHEALHNHYTQKSLSLSP

SEQ ID NO: 406 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLVSKLTVDKSRWQQGNVFSCSVMHEALHNHYTQKSLSLSP

SEQ ID NO: 407 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLVSKLTVDKSRWQQGNVFSCSVMHEALHNHYTQKSLSLSP

SEQ ID NO: 408 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLVSKLTVDKSRWQQGNVFSCSVMHEALHNHYTQKSLSLSP

SEQ ID NO: 409 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCAVKGFYPSDIAVEWESNGQPENNYKTTPPVLDSDGSFFLVSKLTVDKSRWQQGNVFSCSVMHEALHNHYTQKSLSLSP

SEQ ID NO: 410 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCAVKGFYPSDIAVEWESNGQPENNYKTTPPVLDSDGSFFLVSKLTVDKSRWQQGNVFSCSVMHEALHNHYTQKSLSLSP

SEQ ID NO: 411 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLHCAVKGFYPSDIAVEWESNGQPENNYKTTPPVLDSDGSFFLVSKLTVDKSRWQQGNVFSCSVMHEALHNHYTQKSLSLSP

SEQ ID NO: 412 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLLSKLTVDKSRWQQGNVFSCSVMHEALHNHYTQKSLSLSP

SEQ ID NO: 413 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLISKLTVDKSRWQQGNVFSCSVMHEALHNHYTQKSLSLSP

SEQ ID NO: 414 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLMSKLTVDKSRWQQGNVFSCSVMHEALHNHYTQKSLSLSP

SEQ ID NO: 415 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLASKLTVDKSRWQQGNVFSCSVMHEALHNHYTQKSLSLSP

SEQ ID NO: 416 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLSSKLTVDKSRWQQGNVFSCSVMHEALHNHYTQKSLSLSP

SEQ ID NO: 417 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLTSKLTVDKSRWQQGNVFSCSVMHEALHNHYTQKSLSLSP

SEQ ID NO: 418 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLCSKLTVDKSRWQQGNVFSCSVMHEALHNHYTQKSLSLSP

SEQ ID NO: 419 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLHSKLTVDKSRWQQGNVFSCSVMHEALHNHYTQKSLSLSP

SEQ ID NO: 420 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLNSKLTVDKSRWQQGNVFSCSVMHEALHNHYTQKSLSLSP

SEQ ID NO: 421 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLCSKLTVDKSRWQQGNVFSCSVMHEALHNHYTQKSLSLSP

SEQ ID NO: 422 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLCCAVKGFYPSDIAVEWESNGQPENNYKTTPPVLDSDGSFFLCSKLTVDKSRWQQGNVFSCSVMHEALHNHYTQKSLSLSP

SEQ ID NO: 423 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLLSKLTVDKSRWQQGNVFSCSVMHEALHNHYTQKSLSLSP

SEQ ID NO: 424 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLLSKLTVDKSRWQQGNVFSCSVMHEALHNHYTQKSLSLSP

SEQ ID NO: 425 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLLSKLTVDKSRWQQGNVFSCSVMHEALHNHYTQKSLSLSP

SEQ ID NO: 426 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLLSKLTVDKSRWQQGNVFSCSVMHEALHNHYTQKSLSLSP

SEQ ID NO: 427 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLLSKLTVDKSRWQQGNVFSCSVMHEALHNHYTQKSLSLSP

SEQ ID NO: 428 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLISKLTVDKSRWQQGNVFSCSVMHEALHNHYTQKSLSLSP

SEQ ID NO: 429 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLISKLTVDKSRWQQGNVFSCSVMHEALHNHYTQKSLSLSP

SEQ ID NO: 430 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLISKLTVDKSRWQQGNVFSCSVMHEALHNHYTQKSLSLSP

SEQ ID NO: 431 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLISKLTVDKSRWQQGNVFSCSVMHEALHNHYTQKSLSLSP

SEQ ID NO: 432 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLMSKLTVDKSRWQQGNVFSCSVMHEALHNHYTQKSLSLSP

SEQ ID NO: 433 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLMSKLTVDKSRWQQGNVFSCSVMHEALHNHYTQKSLSLSP

SEQ ID NO: 434 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLMSKLTVDKSRWQQGNVFSCSVMHEALHNHYTQKSLSLSP

SEQ ID NO: 435 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLMSKLTVDKSRWQQGNVFSCSVMHEALHNHYTQKSLSLSP

SEQ ID NO: 436 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLMSKLTVDKSRWQQGNVFSCSVMHEALHNHYTQKSLSLSP

SEQ ID NO: 437 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLASKLTVDKSRWQQGNVFSCSVMHEALHNHYTQKSLSLSP

SEQ ID NO: 438 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLASKLTVDKSRWQQGNVFSCSVMHEALHNHYTQKSLSLSP

SEQ ID NO: 439 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLASKLTVDKSRWQQGNVFSCSVMHEALHNHYTQKSLSLSP

SEQ ID NO: 440 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLASKLTVDKSRWQQGNVFSCSVMHEALHNHYTQKSLSLSP

SEQ ID NO: 441 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLASKLTVDKSRWQQGNVFSCSVMHEALHNHYTQKSLSLSP

SEQ ID NO: 442 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLTSKLTVDKSRWQQGNVFSCSVMHEALHNHYTQKSLSLSP

SEQ ID NO: 443 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLTSKLTVDKSRWQQGNVFSCSVMHEALHNHYTQKSLSLSP

SEQ ID NO: 444 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLTSKLTVDKSRWQQGNVFSCSVMHEALHNHYTQKSLSLSP

SEQ ID NO: 445 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLTSKLTVDKSRWQQGNVFSCSVMHEALHNHYTQKSLSLSP

SEQ ID NO: 446 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLTSKLTVDKSRWQQGNVFSCSVMHEALHNHYTQKSLSLSP

SEQ ID NO: 447 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLCSKLTVDKSRWQQGNVFSCSVMHEALHNHYTQKSLSLSP

SEQ ID NO: 448 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLCSKLTVDKSRWQQGNVFSCSVMHEALHNHYTQKSLSLSP

SEQ ID NO: 449 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLCSKLTVDKSRWQQGNVFSCSVMHEALHNHYTQKSLSLSP

SEQ ID NO: 450 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLCSKLTVDKSRWQQGNVFSCSVMHEALHNHYTQKSLSLSP

SEQ ID NO: 451 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLHSKLTVDKSRWQQGNVFSCSVMHEALHNHYTQKSLSLSP

SEQ ID NO: 452 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLHSKLTVDKSRWQQGNVFSCSVMHEALHNHYTQKSLSLSP

SEQ ID NO: 453 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLHSKLTVDKSRWQQGNVFSCSVMHEALHNHYTQKSLSLSP

SEQ ID NO: 454 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLHSKLTVDKSRWQQGNVFSCSVMHEALHNHYTQKSLSLSP

SEQ ID NO: 455 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLNSKLTVDKSRWQQGNVFSCSVMHEALHNHYTQKSLSLSP

SEQ ID NO: 456 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLNSKLTVDKSRWQQGNVFSCSVMHEALHNHYTQKSLSLSP

SEQ ID NO: 457 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLNSKLTVDKSRWQQGNVFSCSVMHEALHNHYTQKSLSLSP

SEQ ID NO: 458 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLNSKLTVDKSRWQQGNVFSCSVMHEALHNHYTQKSLSLSP

SEQ ID NO: 459 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCSVKGFYPSDIAVEWESNGQPENNYKTTPPVLDSDGSFFLQSKLTVDKSRWQQGNVFSCSVMHEALHNHYTQKSLSLSP

SEQ ID NO: 460 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLQSKLTVDKSRWQQGNVFSCSVMHEALHNHYTQKSLSLSP

SEQ ID NO: 461 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLQSKLTVDKSRWQQGNVFSCSVMHEALHNHYTQKSLSLSP

SEQ ID NO: 462 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLQSKLTVDKSRWQQGNVFSCSVMHEALHNHYTQKSLSLSP

SEQ ID NO: 463 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCGVKGFYPSDIAVEWESNGQPENNYKTTPPVLDSDGSFFLQSKLTVDKSRWQQGNVFSCSVMHEALHNHYTQKSLSLSP

SEQ ID NO: 464 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLLSKLTVDKSRWQQGNVFSCSVMHEALHNHYTQKSLSLSP

SEQ ID NO: 465 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLLSKLTVDKSRWQQGNVFSCSVMHEALHNHYTQKSLSLSP

SEQ ID NO: 466 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLLSKLTVDKSRWQQGNVFSCSVMHEALHNHYTQKSLSLSP

SEQ ID NO: 467 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLLSKLTVDKSRWQQGNVFSCSVMHEALHNHYTQKSLSLSP

SEQ ID NO: 468 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLISKLTVDKSRWQQGNVFSCSVMHEALHNHYTQKSLSLSP

SEQ ID NO: 469 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLISKLTVDKSRWQQGNVFSCSVMHEALHNHYTQKSLSLSP

SEQ ID NO: 470 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLISKLTVDKSRWQQGNVFSCSVMHEALHNHYTQKSLSLSP

SEQ ID NO: 471 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLISKLTVDKSRWQQGNVFSCSVMHEALHNHYTQKSLSLSP

SEQ ID NO: 472 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLMSKLTVDKSRWQQGNVFSCSVMHEALHNHYTQKSLSLSP

SEQ ID NO: 473 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLASKLTVDKSRWQQGNVFSCSVMHEALHNHYTQKSLSLSP

SEQ ID NO: 474 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLASKLTVDKSRWQQGNVFSCSVMHEALHNHYTQKSLSLSP

SEQ ID NO: 475 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLTSKLTVDKSRWQQGNVFSCSVMHEALHNHYTQKSLSLSP

SEQ ID NO: 476 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLTSKLTVDKSRWQQGNVFSCSVMHEALHNHYTQKSLSLSP

SEQ ID NO: 477 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLTSKLTVDKSRWQQGNVFSCSVMHEALHNHYTQKSLSLSP

SEQ ID NO: 478 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLTSKLTVDKSRWQQGNVFSCSVMHEALHNHYTQKSLSLSP

SEQ ID NO: 479 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLTSKLTVDKSRWQQGNVFSCSVMHEALHNHYTQKSLSLSP

SEQ ID NO: 480 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLCSKLTVDKSRWQQGNVFSCSVMHEALHNHYTQKSLSLSP

SEQ ID NO: 481 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLCSKLTVDKSRWQQGNVFSCSVMHEALHNHYTQKSLSLSP

SEQ ID NO: 482 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLCSKLTVDKSRWQQGNVFSCSVMHEALHNHYTQKSLSLSP

SEQ ID NO: 483 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLHSKLTVDKSRWQQGNVFSCSVMHEALHNHYTQKSLSLSP

SEQ ID NO: 484 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLHSKLTVDKSRWQQGNVFSCSVMHEALHNHYTQKSLSLSP

SEQ ID NO: 485 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLHSKLTVDKSRWQQGNVFSCSVMHEALHNHYTQKSLSLSP

SEQ ID NO: 486 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLHSKLTVDKSRWQQGNVFSCSVMHEALHNHYTQKSLSLSP

SEQ ID NO: 487 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLHSKLTVDKSRWQQGNVFSCSVMHEALHNHYTQKSLSLSP

SEQ ID NO: 488 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLNSKLTVDKSRWQQGNVFSCSVMHEALHNHYTQKSLSLSP

SEQ ID NO: 489 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLNSKLTVDKSRWQQGNVFSCSVMHEALHNHYTQKSLSLSP

SEQ ID NO: 490 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLNSKLTVDKSRWQQGNVFSCSVMHEALHNHYTQKSLSLSP

SEQ ID NO: 491 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLNSKLTVDKSRWQQGNVFSCSVMHEALHNHYTQKSLSLSP

SEQ ID NO: 492 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLNSKLTVDKSRWQQGNVFSCSVMHEALHNHYTQKSLSLSP

SEQ ID NO: 493 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLQSKLTVDKSRWQQGNVFSCSVMHEALHNHYTQKSLSLSP

SEQ ID NO: 494 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLQSKLTVDKSRWQQGNVFSCSVMHEALHNHYTQKSLSLSP

SEQ ID NO: 495 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLQSKLTVDKSRWQQGNVFSCSVMHEALHNHYTQKSLSLSP

SEQ ID NO: 496 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLQSKLTVDKSRWQQGNVFSCSVMHEALHNHYTQKSLSLSP

SEQ ID NO: 497 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCTVKGFYPSDIAVEWESNGQPENNYKTTPPVLDSDGSFFLISKLTVDKSRWQQGNVFSCSVMHEALHNHYTQKSLSLSP

SEQ ID NO: 498 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCCVKGFYPSDIAVEWESNGQPENNYKTTPPVLDSDGSFFLHSKLTVDKSRWQQGNVFSCSVMHEALHNHYTQKSLSLSP

SEQ ID NO: 499 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCVVKGFYPSDIAVEWESNGQPENNYKTTPPVLDSDGSFFLNSKLTVDKSRWQQGNVFSCSVMHEALHNHYTQKSLSLSP

SEQ ID NO: 500 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLLSKLTVDKSRWQQGNVFSCSVMHEALHNHYTQKSLSLSP

SEQ ID NO: 501 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLCSKLTVDKSRWQQGNVFSCSVMHEALHNHYTQKSLSLSP

SEQ ID NO: 502 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLLSKLTVDKSRWQQGNVFSCSVMHEALHNHYTQKSLSLSP

SEQ ID NO: 503 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLLSKLTVDKSRWQQGNVFSCSVMHEALHNHYTQKSLSLSP

SEQ ID NO: 504 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLLSKLTVDKSRWQQGNVFSCSVMHEALHNHYTQKSLSLSP

SEQ ID NO: 505 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLLSKLTVDKSRWQQGNVFSCSVMHEALHNHYTQKSLSLSP

SEQ ID NO: 506 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLLSKLTVDKSRWQQGNVFSCSVMHEALHNHYTQKSLSLSP

SEQ ID NO: 507 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLISKLTVDKSRWQQGNVFSCSVMHEALHNHYTQKSLSLSP

SEQ ID NO: 508 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLISKLTVDKSRWQQGNVFSCSVMHEALHNHYTQKSLSLSP

SEQ ID NO: 509 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLISKLTVDKSRWQQGNVFSCSVMHEALHNHYTQKSLSLSP

SEQ ID NO: 510 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLISKLTVDKSRWQQGNVFSCSVMHEALHNHYTQKSLSLSP

SEQ ID NO: 511 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLISKLTVDKSRWQQGNVFSCSVMHEALHNHYTQKSLSLSP

SEQ ID NO: 512 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLMSKLTVDKSRWQQGNVFSCSVMHEALHNHYTQKSLSLSP

SEQ ID NO: 513 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLMSKLTVDKSRWQQGNVFSCSVMHEALHNHYTQKSLSLSP

SEQ ID NO: 514 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLMSKLTVDKSRWQQGNVFSCSVMHEALHNHYTQKSLSLSP

SEQ ID NO: 515 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLMSKLTVDKSRWQQGNVFSCSVMHEALHNHYTQKSLSLSP

SEQ ID NO: 516 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLMSKLTVDKSRWQQGNVFSCSVMHEALHNHYTQKSLSLSP

SEQ ID NO: 517 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLASKLTVDKSRWQQGNVFSCSVMHEALHNHYTQKSLSLSP

SEQ ID NO: 518 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLASKLTVDKSRWQQGNVFSCSVMHEALHNHYTQKSLSLSP

SEQ ID NO: 519 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLASKLTVDKSRWQQGNVFSCSVMHEALHNHYTQKSLSLSP

SEQ ID NO: 520 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLASKLTVDKSRWQQGNVFSCSVMHEALHNHYTQKSLSLSP

SEQ ID NO: 521 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLASKLTVDKSRWQQGNVFSCSVMHEALHNHYTQKSLSLSP

SEQ ID NO: 522 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLTSKLTVDKSRWQQGNVFSCSVMHEALHNHYTQKSLSLSP

SEQ ID NO: 523 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLTSKLTVDKSRWQQGNVFSCSVMHEALHNHYTQKSLSLSP

SEQ ID NO: 524 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLTSKLTVDKSRWQQGNVFSCSVMHEALHNHYTQKSLSLSP

SEQ ID NO: 525 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLTSKLTVDKSRWQQGNVFSCSVMHEALHNHYTQKSLSLSP

SEQ ID NO: 526 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLTSKLTVDKSRWQQGNVFSCSVMHEALHNHYTQKSLSLSP

SEQ ID NO: 527 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLCSKLTVDKSRWQQGNVFSCSVMHEALHNHYTQKSLSLSP

SEQ ID NO: 528 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLCSKLTVDKSRWQQGNVFSCSVMHEALHNHYTQKSLSLSP

SEQ ID NO: 529 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLCSKLTVDKSRWQQGNVFSCSVMHEALHNHYTQKSLSLSP

SEQ ID NO: 530 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLCSKLTVDKSRWQQGNVFSCSVMHEALHNHYTQKSLSLSP

SEQ ID NO: 531 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLHSKLTVDKSRWQQGNVFSCSVMHEALHNHYTQKSLSLSP

SEQ ID NO: 532 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLHSKLTVDKSRWQQGNVFSCSVMHEALHNHYTQKSLSLSP

SEQ ID NO: 533 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLHSKLTVDKSRWQQGNVFSCSVMHEALHNHYTQKSLSLSP

SEQ ID NO: 534 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLHSKLTVDKSRWQQGNVFSCSVMHEALHNHYTQKSLSLSP

SEQ ID NO: 535 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLNSKLTVDKSRWQQGNVFSCSVMHEALHNHYTQKSLSLSP

SEQ ID NO: 536 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLNSKLTVDKSRWQQGNVFSCSVMHEALHNHYTQKSLSLSP

SEQ ID NO: 537 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLNSKLTVDKSRWQQGNVFSCSVMHEALHNHYTQKSLSLSP

SEQ ID NO: 538 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLNSKLTVDKSRWQQGNVFSCSVMHEALHNHYTQKSLSLSP

SEQ ID NO: 539 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCSVKGFYPSDIAVEWESNGQPENNYKTTPPVLDSDGSFFLQSKLTVDKSRWQQGNVFSCSVMHEALHNHYTQKSLSLSP

SEQ ID NO: 540 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLQSKLTVDKSRWQQGNVFSCSVMHEALHNHYTQKSLSLSP

SEQ ID NO: 541 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLQSKLTVDKSRWQQGNVFSCSVMHEALHNHYTQKSLSLSP

SEQ ID NO: 542 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLQSKLTVDKSRWQQGNVFSCSVMHEALHNHYTQKSLSLSP

SEQ ID NO: 543 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCGVKGFYPSDIAVEWESNGQPENNYKTTPPVLDSDGSFFLQSKLTVDKSRWQQGNVFSCSVMHEALHNHYTQKSLSLSP

SEQ ID NO: 544 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCCVKGFYPSDIAVEWESNGQPENNYKTTPPVLDSDGSFFLCSKLTVDKSRWQQGNVFSCSVMHEALHNHYTQKSLSLSP

SEQ ID NO: 545 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLISKLTVDKSRWQQGNVFSCSVMHEALHNHYTQKSLSLSP

SEQ ID NO: 546 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCVVKGFYPSDIAVEWESNGQPENNYKTTPPVLDSDGSFFLHSKLTVDKSRWQQGNVFSCSVMHEALHNHYTQKSLSLSP

SEQ ID NO: 547 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLFCTVKGFYPSDIAVEWESNGQPENNYKTTPPVLDSDGSFFLNSKLTVDKSRWQQGNVFSCSVMHEALHNHYTQKSLSLSP

SEQ ID NO: 548 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLCSKLTVDKSRWQQGNVFSCSVMHEALHNHYTQKSLSLSP

SEQ ID NO: 549 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLQSKLTVDKSRWQQGNVFSCSVMHEALHNHYTQKSLSLSP

SEQ ID NO: 550 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLMSKLTVDKSRWQQGNVFSCSVMHEALHNHYTQKSLSLSP

SEQ ID NO: 551 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLMSKLTVDKSRWQQGNVFSCSVMHEALHNHYTQKSLSLSP

SEQ ID NO: 552 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLMSKLTVDKSRWQQGNVFSCSVMHEALHNHYTQKSLSLSP

SEQ ID NO: 553 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCVVKGFYPSDIAVEWESNGQPENNYKTTPPVLDSDGSFFLMSKLTVDKSRWQQGNVFSCSVMHEALHNHYTQKSLSLSP

SEQ ID NO: 554 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCSVKGFYPSDIAVEWESNGQPENNYKTTPPVLDSDGSFFLASKLTVDKSRWQQGNVFSCSVMHEALHNHYTQKSLSLSP

SEQ ID NO: 555 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCTVKGFYPSDIAVEWESNGQPENNYKTTPPVLDSDGSFFLASKLTVDKSRWQQGNVFSCSVMHEALHNHYTQKSLSLSP

SEQ ID NO: 556 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCCVKGFYPSDIAVEWESNGQPENNYKTTPPVLDSDGSFFLASKLTVDKSRWQQGNVFSCSVMHEALHNHYTQKSLSLSP

SEQ ID NO: 557 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLYCGVKGFYPSDIAVEWESNGQPENNYKTTPPVLDSDGSFFLQSKLTVDKSRWQQGNVFSCSVMHEALHNHYTQKSLSLSP

SEQ ID NO: 558 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLISDGSFTLYSYLTVDKSRWQQGNVFSCSVMHEALHNHYTQKSLSLSP

SEQ ID NO: 559 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLLSDGSFFLASLLTVDKSRWQQGNVFSCSVMHEALHNHYTQKSLSLSP

SEQ ID NO: 560 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLISDGSFFLASILTVDKSRWQQGNVFSCSVMHEALHNHYTQKSLSLSP

SEQ ID NO: 561 - protein, 447 aa, Homo sapiens
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 562 - protein, 447 aa, Homo sapiens
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCARWTGRTDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 563 - protein, 447 aa, Homo sapiens; identical to SEQ ID NO: 79

SEQ ID NO: 564 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 565 - protein, 447 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 566 - protein, 447 aa, Homo sapiens; identical to SEQ ID NO: 82

SEQ ID NO: 567 - protein, 450 aa, Homo sapiens
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREEGGWIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 568 - protein, 447 aa, Homo sapiens
QVQLVQSGSELKKPGASVKVSCKASGYTFTQNNMDWVRQAPGQGLEWMGDINTRSGGVIYNEEFQDRLIMTVDKSTDTAYMELSSLRSEDTATYHCARRKSYGYYLDVWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 569 - protein, 447 aa, Homo sapiens; identical to SEQ ID NO: 78

SEQ ID NO: 570 - protein, 447 aa, Homo sapiens
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 571 - protein, 447 aa, Homo sapiens
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 572 - protein, 447 aa, Homo sapiens
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 573 - protein, 450 aa, Homo sapiens
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 574 - protein, 450 aa, Homo sapiens
QVQLVESGGGVVQPGGSLRLSCAASGFTFSNAWMHWVRQAPGKGLEWVAQIKDKSQNYATYVAESVKGRFTISRADSKNSIYLQMNSLKTEDTAVYYCRYVHYAAGYGVDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 575 - protein, 448 aa, Homo sapiens
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 576 - protein, 448 aa, Homo sapiens
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTWPPVLDSDGSFALYSRLTVDKSRWQEGNVFSCSVMHEALHNHYTQKSLSLSL

SEQ ID NO: 577 - protein, 219 aa, Homo sapiens
DIVMTQSPLSLPVTPGEPASISCRSSQPLVHSNRNTYLHWYQQKPGQAPRLLIYKVSNRFSGVPDRFSGSGSGTDFTLKISRVEAEDVGVYYCGQGTQVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 578 - protein, 214 aa, Homo sapiens
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 579 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 580 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 581 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 582 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 583 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTFPPVLDSDGSFALYSKLTVDKSRWQQGNVFSCSVMHEALHNHYTQKSLSLSP

SEQ ID NO: 584 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSELTVDKSRWQQGNVFSCSVMHEALHNHYTQESLSLSP

SEQ ID NO: 585 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVYTLPPYREEMTKNQVSLYCLVKGFYPSDIAVEWESNGQPENNYKTTPPVLDSDGSFFLTSKLTVDKSRWQQGNVFSCSVMHEALHNHYTQKSLSLSP

SEQ ID NO: 586 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYLTWPPVLDSDGSFALVSKLTVDKSRWQQGNVFSCSVMHEALHNHYTQKSLSLSP

SEQ ID NO: 587 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 588 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 589 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRECMTKNQVSLTCLVKGFYPSDIAVEWESNGQPENNYDTTPPVLKSDGSFFLYSKLTVDKSRWQQGNVFSCSVMHEALHNHYTQKSLSLSP

SEQ ID NO: 590 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTDNQVSLVCLVKGFYPSDIAVEWESNGQPENNYKTTPPVLMSDGSFFLASVLTVDKSRWQQGNVFSCSVMHEALHNHYTQKSLSLSP

SEQ ID NO: 591 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSDLTVDKSRWQQGNVFSCSVMHEALHNHYTQKSLSLSP

SEQ ID NO: 592 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTENQVSLTCLVKGFYPSDIAVEWESNGQPENNYKTTPPVLMSDGSFFLASVLTVDKSRWQQGNVFSCSVMHEALHNHYTQKSLSLSP

SEQ ID NO: 593 - protein, 347 aa, Homo sapiens
EVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLLCLVKGFYPSDIAVEWESNGQPENNYLTWPPVLDSDGSFALVSKLTVDKSRWQQGNVFSCSVMHEALHNHYTQKSLSLSP

SEQ ID NO: 594 - protein, 225 aa, Homo sapiens
DKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 595 - protein, 225 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 596 - protein, 225 aa, Homo sapiens
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 597 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSP

SEQ ID NO: 598 - protein, 225 aa, Homo sapiens
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 599 - protein, 225 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 600 - protein, 225 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTWPPVLDSDGSFALYSKLTVDKSRWQQGNVFSCSVMHEALHNHYTQKSLSLSP

SEQ ID NO: 601 - protein, 225 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLWCAVKGFYPSDIAVEWESNGQPENNYKTTPPVLDSDGSFFLVSKLTVDKSRWQQGNVFSCSVMHEALHNHYTQKSLSLSP

SEQ ID NO: 602 - protein, 225 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPRVYTLPPSREEMTENQVSLTCLVKGFYPSDIAVEWESNGQPENNYKTTPPVLVSDGSFTLYSWLTVDKSRWQQGNVFSCSVMHEALHNHYTQKSLSLSP

SEQ ID NO: 603 - protein, 225 aa, Homo sapiens
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 604 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTWPPVLDSDGSFALYSKLTVDKSRWQQGNVFSCSVMHGALHNHYTQKYLSLSP

SEQ ID NO: 605 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLWCAVKGFYPSDIAVEWESNGQPENNYKTTPPVLDSDGSFFLVSKLTVDKSRWQQGNVFSCSVMHGALHNHYTQKYLSLSP

SEQ ID NO: 606 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPRVYTLPPSREEMTENQVSLTCLVKGFYPSDIAVEWESNGQPENNYKTTPPVLVSDGSFTLYSWLTVDKSRWQQGNVFSCSVMHGALHNHYTQKYLSLSP

SEQ ID NO: 607 - protein, 443 aa, Homo sapiens
QVQLVQSGAEVKKPGASVTVSCKASGYTFTDYEMHWIRQPPGEGLEWIGAIDGPTPDTAYSEKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 608 - protein, 443 aa, Homo sapiens
QVQLVQSGAEVKKPGASVTVSCKASGYTFTDYEMHWIRQPPGEGLEWIGAIDGPTPDTAYSEKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYDTTPPVLKSDGSFFLYSKLTVDKSRWQQGNVFSCSVMHEALHNHYTQESLSLSP

SEQ ID NO: 609 - protein, 444 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 610 - protein, 444 aa, Homo sapiens
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCAVKGFYPSDIAVEWESNGQPENNYKTTPPVLDSDGSFFLVSRLTVDKSRWQEGNVFSCSVMHEALHNHYTQKSLSLSL

SEQ ID NO: 611 - RNA, 1407 nt, Homo sapiens
  source 1..1407: mol_type = mRNA
atggagtttgggctgtcatgggtctttctggtcgctctgctgcggggagtgcagtgtcaggtccagctggtcgaatcaggcggaggcgtggtccagcccggagggtctctgagactgagttgcgccgcttcaggcttcacattttctaacgcctggatgcactgggtgcgacaggctcctggcaaagggctggagtgggtcgcacagatcaaggacaaatcccagaattacgctacttacgttgcagaatccgtgaaggggaggttcaccatttcccgcgccgattccaaaaactccatctacctgcagatgaatagtctgaagaccgaggacacagccgtgtactattgtagatatgtccattacgcagccggctacggagtggacatctggggacagggaactacagtcacagtctcatccgcttccaccaagggcccatcggtcttccccctggcaccctcctccaagtccacctctgggggcacagcggccctgggctgcctggtcaaggactacttccccgaaccggtgacggtgtcgtggaactcaggcgccctgacctccggcgtgcacaccttcccggctgtcctacagtcctcaggactctactccctctcctccgtggtgaccgtgccctcctcgtccttgggcacccagacctacatctgcaacgtgaatcacaagccctccaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgtcccacgaagaccctgaggtcaagttcaactggtacgtggacggcgtggaggtgcataatgccaagacaaagccgcgggaggagcagtacaactccacgtaccgtgtggtctccgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggaaatgaccaagaaccaggtctccctgacctgcctggtcaaaggcttctatccctccgacatcgccgtggagtgggagtccaatgggcagccggagaacaactacaagaccacgcctcccgtgctggactccgacggctccttcttcctctactccaagctcaccgtggacaagtccaggtggcagcaggggaacgtcttctcatgctccgtgatgcatgaggctctgcacaaccactacacgcagaagtcgctctccctgtctccg

SEQ ID NO: 612 - RNA, 1386 nt, Homo sapiens
  source 1..1386: mol_type = mRNA
atgggctggtcctgcatcatcctgtttctggtggccaccgctaccggcgtccatagccaggtgcagctggtgcagtctggagctgaggtgaagaagcctggggcctcagtgaccgtctcctgcaaggcttctggatacaccttcaccgactatgaaatgcactggatacgacagccccctggagaagggcttgagtggatcggagctattgatggtccaactcctgatactgcctacagtgagaagttcaagggcagagtcacgctgaccgcggacaaatccacgagcacagcctacatggagctgagcagcctgacatctgaggacacggccgtgtattactgtacaagattctactcctatacttactggggccagggaaccctggtcaccgtctcctcagcttccaccaagggcccatcggtcttccccctggcaccctcctccaagtccacctctgggggcacagcggccctgggctgcctggtcaaggactacttccccgaaccggtgacggtgtcgtggaactcaggcgccctgacctccggcgtgcacaccttcccggctgtcctacagtcctcaggactctactccctctcctccgtggtgaccgtgccctcctcgtccttgggcacccagacctacatctgcaacgtgaatcacaagccctccaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgtcccacgaagaccctgaggtcaagttcaactggtacgtggacggcgtggaggtgcataatgccaagacaaagccgcgggaggagcagtacaactccacgtaccgtgtggtctccgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggaaatgaccaagaaccaggtctccctgacctgcctggtcaaaggcttctatccctccgacatcgccgtggagtgggagtccaatgggcagccggagaacaactacaagaccacgcctcccgtgctggactccgacggctccttcttcctctactccaagctcaccgtggacaagtccaggtggcagcaggggaacgtcttctcatgctccgtgatgcatgaggctctgcacaaccactacacgcagaagtcgctctccctgtctccg

SEQ ID NO: 613 - RNA, 1407 nt, Homo sapiens
  source 1..1407: mol_type = mRNA
atggagtttgggctgtcatgggtctttctggtcgctctgctgcggggagtgcagtgtcaggtccagctggtcgaatcaggcggaggcgtggtccagcccggagggtctctgagactgagttgcgccgcttcaggcttcacattttctaacgcctggatgcactgggtgcgacaggctcctggcaaagggctggagtgggtcgcacagatcaaggacaaatcccagaattacgctacttacgttgcagaatccgtgaaggggaggttcaccatttcccgcgccgattccaaaaactccatctacctgcagatgaatagtctgaagaccgaggacacagccgtgtactattgtagatatgtccattacgcagccggctacggagtggacatctggggacagggaactacagtcacagtctcatccgcttccaccaagggcccatcggtcttccccctggcaccctcctccaagtccacctctgggggcacagcggccctgggctgcctggtcaaggactacttccccgaaccggtgacggtgtcgtggaactcaggcgccctgacctccggcgtgcacaccttcccggctgtcctacagtcctcaggactctactccctctcctccgtggtgaccgtgccctcctcgtccttgggcacccagacctacatctgcaacgtgaatcacaagccctccaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgtcccacgaagaccctgaggtcaagttcaactggtacgtggacggcgtggaggtgcataatgccaagacaaagccgcgggaggagcagtacaactccacgtaccgtgtggtctccgtcctcaccgtcctgcaccaggactggctgaatggcaaggagtacaagtgcaaggtctccaacaaagccctcccagcccccatcgagaaaaccatctccaaagccaaagggcagccccgagaaccaagagtgtacaccctgcccccatcccgggaggaaatgaccgagaaccaggtctccctgacctgcctggtcaaaggcttctatccctccgacatcgccgtggagtgggagtccaatgggcagccggagaacaactacaagaccacgcctcccgtgctggtgtccgacggctccttcaccctctactcctggctcaccgtggacaagtccaggtggcagcaggggaacgtcttctcatgctccgtgatgcatgaggctctgcacaaccactacacgcagaagtcgctctccctgtctccg

SEQ ID NO: 614 - RNA, 1386 nt, Homo sapiens
  source 1..1386: mol_type = mRNA
atgggctggtcctgcatcatcctgtttctggtggccaccgctaccggcgtccatagccaggtgcagctggtgcagtctggagctgaggtgaagaagcctggggcctcagtgaccgtctcctgcaaggcttctggatacaccttcaccgactatgaaatgcactggatacgacagccccctggagaagggcttgagtggatcggagctattgatggtccaactcctgatactgcctacagtgagaagttcaagggcagagtcacgctgaccgcggacaaatccacgagcacagcctacatggagctgagcagcctgacatctgaggacacggccgtgtattactgtacaagattctactcctatacttactggggccagggaaccctggtcaccgtctcctcagcttccaccaagggcccatcggtcttccccctggcaccctcctccaagtccacctctgggggcacagcggccctgggctgcctggtcaaggactacttccccgaaccggtgacggtgtcgtggaactcaggcgccctgacctccggcgtgcacaccttcccggctgtcctacagtcctcaggactctactccctctcctccgtggtgaccgtgccctcctcgtccttgggcacccagacctacatctgcaacgtgaatcacaagccctccaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgtcccacgaagaccctgaggtcaagttcaactggtacgtggacggcgtggaggtgcataatgccaagacaaagccgcgggaggagcagtacaactccacgtaccgtgtggtctccgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggaaagaaatgaccaagaaccaggtctccctgacctgcctggtcaaaggcttctatccctccgacatcgccgtggagtgggagtccaatgggcagccggagaacaactacgacaccacgcctcccgtcctgaagtccgacggctccttcttcctctactccaagctcaccgtggacaagtccaggtggcagcaggggaacgtcttctcatgctccgtgatgcatgaggctctgcacaaccactacacgcaggagtcgctctccctgtctccg

SEQ ID NO: 615 - RNA, 1401 nt, Homo sapiens
  source 1..1401: mol_type = mRNA
atggagtttgggctgagctgggttttcctcgttgctcttttaagaggtgtccagtgtcaggtgcagctggtggagtctgggggaggcttggtccagcctggggggtccctgagactctcctgtgcagcctctggattcaccttcagttactatgacatccagtgggtccgccaggctccaggcaaggggctggagtgggtgtcatccattagtcctagtggtcagtccacttactatcgacgcgaggtaaagggccgattcaccatctccagggacaactccaagaacacactgtatctgcaaatgaactccctgagagccgaggacacggccgtgtattactgtgcgagacggaccgggcgggagtacggaggtggctggtactttgactactggggccaaggaaccctggtcaccgtgtcctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctg

SEQ ID NO: 616 - RNA, 1389 nt, Homo sapiens
  source 1..1389: mol_type = mRNA
atggactggacctggaggatactcttcttggtggcagcagcgacaggtgcccactcccaggtgcagctggtgcagtctgggtctgagttgaagaagcctggggcctcggtgaaggtctcctgcaaggcttctggatacaccttcaccgacaacaacatggactgggtgcgacaggcccctggacaagggcttgagtggatgggagatattaatactagaagtggtggttctatctacaacgaggagtttcaggacagagtcatcatgaccgtggacaagtccacggacacagcctacatggaattgtcctccctgagatctgaggacacggccacttatcactgcgcgagaaggaagtcctacggctactaccttgacgagtggggcgagggaaccctggtcaccgtctcctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctg

SEQ ID NO: 617 - RNA, 1401 nt, Homo sapiens
  source 1..1401: mol_type = mRNA
atggagtttgggctgagctgggttttcctcgttgctcttttaagaggtgtccagtgtcaggtgcagctggtggagtctgggggaggcttggtccagcctggggggtccctgagactctcctgtgcagcctctggattcaccttcagttactatgacatccagtgggtccgccaggctccaggcaaggggctggagtgggtgtcatccattagtcctagtggtcagtccacttactatcgacgcgaggtaaagggccgattcaccatctccagggacaactccaagaacacactgtatctgcaaatgaactccctgagagccgaggacacggccgtgtattactgtgcgagacggaccgggcgggagtacggaggtggctggtactttgactactggggccaaggaaccctggtcaccgtgtcctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagacctggcctcccgtgctggactccgacggctccttcgccctctacagcaggctaaccgtggacaagagcaggtggcaggaggggaatgtcttctcatgctccgtgatgcatgaggctctgcacaaccactacacacagaagagcctctccctgtctctg

SEQ ID NO: 618 - RNA, 1389 nt, Homo sapiens
  source 1..1389: mol_type = mRNA
atggactggacctggaggatactcttcttggtggcagcagcgacaggtgcccactcccaggtgcagctggtgcagtctgggtctgagttgaagaagcctggggcctcggtgaaggtctcctgcaaggcttctggatacaccttcaccgacaacaacatggactgggtgcgacaggcccctggacaagggcttgagtggatgggagatattaatactagaagtggtggttctatctacaacgaggagtttcaggacagagtcatcatgaccgtggacaagtccacggacacagcctacatggaattgtcctccctgagatctgaggacacggccacttatcactgcgcgagaaggaagtcctacggctactaccttgacgagtggggcgagggaaccctggtcaccgtctcctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtggtgcgcagtcaaaggcttctaccccagcgacatcgccgtggagtgggagagcaatgggcagccggagaacaactacaagaccacgcctcccgtgctggactccgacggctccttcttcctcgtcagcaggctaaccgtggacaagagcaggtggcaggaggggaatgtcttctcatgctccgtgatgcatgaggctctgcacaaccactacacacagaagagcctctccctgtctctg

SEQ ID NO: 619 - RNA, 714 nt, Homo sapiens
  source 1..714: mol_type = mRNA
atgggctggtcctgcatcatcctgtttctggtggccaccgctaccggcgtccatagcgatattgtgatgactcagtctccactctccctgcccgtcacccctggagagccggcctccatctcctgcagatctagtcagccccttgtacacagtaataggaacacctatttacattggtaccagcagaagccagggcaggctccaaggctcctgatctataaagtttccaaccgattttctggggtccctgacaggttcagtggcagtggatcaggcacagattttacactgaaaatcagcagagtggaggctgaggatgttggggtttattactgcggccaaggtacacaggttccttacacgtttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggactccaaggactccacctactccctctcctccaccctgacgctgtccaaagcagactacgagaaacacaaagtctacgcctgcgaagtcacccatcagggcctgtcctcgcccgtcacaaagtccttcaacaggggagagtgt

SEQ ID NO: 620 - RNA, 708 nt, Homo sapiens
  source 1..708: mol_type = mRNA
atggacatgagggtccccgctcagctcctggggctcctgctactctggctccgaggtgccagatgtgacatccagatgacccagtctccatcctcactgtctgcatctgtaggagacagagtcaccatcacttgtaaagcaagtcggaatattgagagacagttagcctggtatcagcagaaacctggccaggctcccgaactcctcatttatcaggcatccagaaaggaatccggcgtcccagacaggttcagtggcagtcggtatgggacagacttcactctcaccatctctagtctgcaacctgaagatatcgcaacttactactgtcagcaatattctgaccctcctctcacgttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggactccaaggactccacctactccctctcctccaccctgacgctgtccaaagcagactacgagaaacacaaagtctacgcctgcgaagtcacccatcagggcctgtcctcgcccgtcacaaagtccttcaacaggggagagtgt

(19) United States
(12) Patent Application Publication
(10) Pub. No.: US 2026/0217835 A1
(43) Pub. Date: Jul. 30, 2026

(54) POLYPEPTIDE WITH CONTROLLED ASSOCIATION

(71) Applicant: Chugai Seiyaku Kabushiki Kaisha, Tokyo (JP)

(72) Inventors: Hikaru Koga, Kanagawa (JP); Shun Shimizu, Kanagawa (JP)

(73) Assignee: Chugai Seiyaku Kabushiki Kaisha, Tokyo (JP)

(21) Appl. No.: 19/142,266

(22) PCT Filed: Dec. 27, 2023
(86) PCT No.: PCT/JP2023/046857

§ 371 Date: Jun. 23, 2025

(30) Foreign Application Priority Data
Dec. 27, 2022 (JP) 2022-210580

(51) Int. Cl. C07K 16/28

(52) U.S. Cl. CPC C07K 16/283; C07K 2317/31; C07K 2317/526; C07K 2317/92; C07K 2317/94

(57) ABSTRACT

The present disclosure relates to polypeptides for which association between polypeptides is controlled, and in one embodiment, polypeptides for which association between Fc regions is controlled, methods for producing polypeptides for which association between Fc regions (in one embodiment, CH3 regions) is controlled, methods for controlling association between Fc regions, compositions comprising such an Fc region, compositions comprising a nucleic acid encoding such a polypeptide, and the like.

### TECHNICAL FIELD

The present disclosure relates to polypeptides for which association between polypeptides is controlled, for example, polypeptides for which association between heavy chains or between Fc regions is controlled, methods for producing polypeptides for which association between Fc regions (in one embodiment, CH3 regions) is controlled, methods for controlling association between heavy chains or between Fc regions, compositions comprising a polypeptide having an Fc region for which association is controlled, and compositions containing a nucleic acid encoding such a polypeptide, and the like.

### BACKGROUND ART

Methods for coexpressing multiple antibodies in a single cell are important for efficient production of pharmaceutical mixtures or generation of pharmaceutical mixtures in living organisms using mRNAs, DNA plasmids, gene therapy, or such. However, when antibody A that is composed of HA (heavy chain) and LA (light chain) and antibody B that is composed of HB (heavy chain) and LB (light chain) are coexpressed, eight antibody species in addition to the desired antibodies A and B are formed in a random manner. This leads to production of a heterogeneous pharmaceutical mixture or heterogeneous efficacy. Therefore, there was a need for a technique to suppress the expression of the antibodies other than antibodies A and B and allow antibodies A and B to be expressed efficiently.

As methods for solving this problem, a method that utilizes differences of the specificity of heavy chain association between isotypes such as IgG and IgA, and a method that utilizes the difference of the specificity of heavy chain association between the subclasses IgG1 and IgG3, have been reported (PTL 1). However, since antibodies that are generally used for therapeutic purposes are IgG, there is a demand for a technique that is applicable to all IgG subclasses and enables even antibodies of the same IgG subclass to be coexpressed. To address this problem, there have been reports of methods for promoting the association of heavy chains of the same type, in which amino acid modifications are introduced into the heavy chain CH3 regions to suppress the expression of IgG in which HA and HB are associated (PTLs 2-4). These are all methods for controlling the association of heavy chains of the same type using the attractive and repulsive forces of electric charges, but have actually been tested for limited modifications and their combinations. Thus, they have not been able to achieve efficient coexpression of three or more antibodies. Moreover, these prior studies were intended for simultaneous production of multiple antibodies, not for expression of antibodies in living organisms. In the case of expressing therapeutic antibodies in living organisms, their not associating with endogenous IgG would lead to reduced side effects. However, there has been no report of a technique that achieves coexpression of multiple antibodies that do not associate with endogenous IgG.

### CITATION LIST

### Patent Literature

[PTL 1] WO 2004/009618
[PTL 2] WO 2013/157953
[PTL 3] WO 2014/015804
[PTL 4] WO 2017/205014

### SUMMARY OF INVENTION

### Technical Problem

The present invention was achieved in view of the above circumstances. An objective of the present invention is to provide polypeptides for which association between polypeptides is controlled, methods for producing polypeptides for which association is controlled, methods for controlling the association of polypeptides, nucleic acids encoding polypeptides for which association is controlled, compositions containing such a nucleic acid, and the like.

A further objective of the present invention is, in one embodiment, to provide polypeptides for which association between Fc regions is controlled, methods for producing polypeptides for which association between Fc regions is controlled, expression of polypeptides for which association between Fc regions is controlled in living organisms, and methods for controlling association between Fc regions. Further, an objective of the present disclosure is to provide, in one embodiment, antibodies for which association in the interface between CH3 regions is controlled, and methods for coexpressing two or more antibodies in an efficient manner.

### Solutions to Problem

The present inventors selected CH3, a constant region of the heavy chain, as an inter-Fc region to be subjected to control of association, and conducted dedicated studies on control of the association between Fc regions of the same type. As a result, the inventors discovered that multimers of Fc regions of the same type can be efficiently formed by suppressing association between Fc regions of different types using steric hindrance caused by modification of amino acid residues in the CH3 interface into bulky and small amino acids at the same time or using artificially introduced disulfide linkages, or by introducing novel combinations of modifications utilizing the attractive and repulsive forces of electric charges.

Thus, with the findings of the present inventors, it is possible to control the association between heavy chains of the same type. Moreover, the present disclosure is not only applicable to the control of association between heavy chains of the same type, but is also able to control association between any Fc-containing polypeptides.

The present inventors further confirmed that antibodies for which association between Fc regions is controlled according to the present disclosure retain the functions of Fc.

As stated above, the present inventors successfully developed polypeptides for which association between Fc regions is controlled, thereby completing the present invention.

The present disclosure relates to polypeptides for which association between polypeptides is controlled, methods for producing polypeptides for which association is controlled, methods for controlling association of polypeptides, nucleic acids encoding polypeptides for which association is controlled, compositions containing such a nucleic acid, and such.

In one embodiment, the present disclosure relates to polypeptides for which association between Fc regions is controlled, methods for producing polypeptides for which association between Fc regions is controlled, expression of polypeptides for which association between Fc regions is controlled in living organisms, and methods for controlling association between Fc regions, and more specifically relates to the following:

[1] A nucleic acid encoding a first polypeptide,
wherein the first polypeptide comprises an Fc region into which a modification has been introduced, and
wherein, because of the modification introduced into the Fc region, the first polypeptide associates more readily with a first polypeptide having the modification via the Fc region than with a first polypeptide comprising an Fc region into which the modification has not been introduced.
   [1-1] The aforementioned nucleic acid, wherein the first polypeptide associates less readily with a polypeptide into which the modification has not been introduced than with a polypeptide having the modification.
   [1-1-1] The aforementioned nucleic acid, wherein the first polypeptide less readily forms a heterodimer with another polypeptide into which the modification has not been introduced.
   [1-1-2] The aforementioned nucleic acid, which less readily forms a heterodimer than a homodimer.
   [1-1-3] The aforementioned nucleic acid, wherein the first polypeptide does not associate, or associates less readily, with endogenous IgG or an antibody fragment containing an Fc region thereof.
   [1-2] The aforementioned nucleic acid, wherein the first polypeptides having the modification associate with each other.
   [1-2-1] The aforementioned nucleic acid, wherein the first polypeptide having the modification forms a homomer.
   [1-2-2] The aforementioned nucleic acid, which more readily forms a homodimer than a heterodimer.
   [1-2-3] The aforementioned nucleic acid, wherein the first polypeptide exhibits a stronger homodimerization-promoting ability than a control (e.g., wild-type IgG).
   [1-2-4] The aforementioned nucleic acid, wherein a homodimer of a polypeptide other than the first polypeptide (e.g., a second polypeptide) is further formed.
   [1-2-5] The aforementioned nucleic acid, wherein a homodimer(s) of one or two or more types of polypeptides are formed.
   [1-2-6] The aforementioned nucleic acid, wherein the modifications in the two or more types of polypeptides are different from each other.
   [1-2-7] The aforementioned nucleic acid, wherein the combination of the modifications different from each other is a combination shown in Table 7.
   [1-3] The aforementioned nucleic acid, wherein the first polypeptide having the modification has an increased ability to form an associated multimer with a polypeptide having the modification as compared to a polypeptide into which the modification has not been introduced.
   [1-4] The aforementioned nucleic acid, wherein the nucleic acid is an RNA, a DNA, or a vector or plasmid carrying the nucleic acid.
   [1-5] The aforementioned nucleic acid, wherein the first polypeptide is a polypeptide for which association is controlled. [1-5-1] The aforementioned nucleic acid, wherein the association is association between Fc regions.
   [1-6] The aforementioned nucleic acid, wherein the first polypeptide has CH3.
   [1-6-1] The aforementioned nucleic acid, wherein the association is association in an interface between CH3 regions.
   [1-6-2] The aforementioned nucleic acid, wherein the modification is a modification in CH3.
   [2] The aforementioned nucleic acid, wherein the first polypeptide associates more readily with a first polypeptide having the modification via the Fc region than with a first polypeptide comprising an Fc region into which the modification has not been introduced due to at least one of the following actions resulting from the introduced modification: (1) steric complementarity (also referred to as “steric hindrance”), (2) disulfide linkage (also referred to as “disulfide bond”), and (3) electrostatic charge (also referred to as “charge”).
   [2-1] The aforementioned nucleic acid, wherein the steric complementarity results from the presence of a knob and a hole in the Fc region.
   [2-1-1] The aforementioned nucleic acid, wherein the modification is a modification to introduce a knob and a hole into the Fc region.
   [2-1-2] The aforementioned nucleic acid, wherein the knob and the hole in the Fc region induce the formation of a homodimer of the first polypeptide.
   [2-1-3] The aforementioned nucleic acid, wherein the knob and the hole are all introduced into the first polypeptide.
   [2-1-4] The aforementioned nucleic acid, which associates more readily with a polypeptide having the modification than a polypeptide into which the modification has not been introduced due to at least the combinations of the actions of (1) steric complementarity and (3) electrostatic charge.
   [2-2] The aforementioned nucleic acid, wherein the disulfide linkage is formed by substituting one or more amino acids in the Fc region with cysteine (C).
   [2-2-1] The aforementioned nucleic acid, wherein the modification is a modification to introduce one or more cysteines into the Fc region.
   [2-2-2] The aforementioned nucleic acid, wherein the modification is substitution of one or more amino acids in the Fc region with cysteine.
   [2-2-3] The aforementioned nucleic acid, wherein the disulfide linkage is a linkage between the cysteines modified (introduced) in the Fc region.
   [2-2-4] The aforementioned nucleic acid, which associates more readily with a polypeptide having the modification than a polypeptide into which the modification has not been introduced due to at least the combinations of the actions of (2) disulfide linkage and (3) electrostatic charge.
   [2-3] The aforementioned nucleic acid, wherein the action of the electrostatic charge results from one or more charged amino acids modified (introduced) in the Fc region.
   [2-3-1] The aforementioned nucleic acid, wherein the introduced charged amino acid(s) is a positively charged amino acid and/or a negatively charged amino acid.
   [2-3-2] The aforementioned nucleic acid, wherein at least two amino acids in the Fc region are substituted with a positively charged amino acid and a negatively charged amino acid.
   [2-3-3] The aforementioned nucleic acid, wherein the positively charged amino acid is selected from lysine (K), arginine (R), and histidine (H), and/or the negatively charged amino acid is selected from aspartic acid (D) and glutamic acid (E).
   [2-4] The aforementioned nucleic acid, wherein the action is (a combination of) more than one action (two or three actions) selected from (1) steric complementarity, (2) disulfide linkage, and
   (3) electrostatic charge.
   [3] A composition comprising:
the nucleic acid of [1]; and
a nucleic acid encoding a second polypeptide,
wherein the second polypeptide comprises an Fc region into which a modification has been introduced, and
wherein, because of the modification introduced into the Fc region, the second polypeptide associates more readily with a second polypeptide having the modification via the Fc region than with a second polypeptide comprising an Fc region into which the modification has not been introduced.
   [3-1] The aforementioned composition, wherein the modification in the second polypeptide is different from the modification in the first polypeptide.
   [3-2] The aforementioned composition, wherein the action in the second polypeptide is different from the action in the first polypeptide.
   [3-3] The aforementioned composition, wherein the amino acid sequence of the second polypeptide prior to modification differs from the amino acid sequence of the first polypeptide prior to modification.
   [3-4] The aforementioned composition, which further comprises a nucleic acid encoding a polypeptide different from the first or second polypeptide (in one embodiment, a third and/or fourth polypeptide).
   [4] A composition comprising:
the nucleic acid of [1];
a nucleic acid encoding a second polypeptide; and
a nucleic acid encoding a third polypeptide,
wherein the second polypeptide associates more readily with the third polypeptide than with the second polypeptide.
   [5] A composition comprising:
the nucleic acid of [2]; and
a nucleic acid encoding a second polypeptide, wherein the second polypeptide does not have the modification the first polypeptide has.
   [6] The aforementioned composition, wherein the modification introduced into the first polypeptide is different from the modification introduced into the second polypeptide.
   [7] The aforementioned composition, wherein the second polypeptide associates more readily with a second polypeptide having the modification via the Fc region than with a second polypeptide comprising an Fc region into which the modification has not been introduced due to at least one of the following actions resulting from the introduced modification: (1) steric complementarity, (2) disulfide linkage, and (3) electrostatic charge.
   [7-1] The aforementioned composition, wherein the modification in the second polypeptide is different from the modification in the first polypeptide.
   [7-2] The aforementioned composition, wherein the action in the second polypeptide is different from the action in the first polypeptide.
   [8] The aforementioned nucleic acid or composition, wherein the modification introduced into the Fc region is at least one of the modifications set forth in Table 2.
   [9] The aforementioned nucleic acid or composition, wherein the modification introduced into the Fc region is at least one of the modifications set forth in Table 4.
   [10] The aforementioned nucleic acid or composition, wherein the modification introduced into the first polypeptide and the modification introduced into the second polypeptide are at least one of the combinations of modifications set forth in Table 6.
   [11] The aforementioned nucleic acid or composition, wherein the polypeptide is an antibody.
   [12] The aforementioned nucleic acid or composition, wherein the Fc region is an Fc region of IgG.
   [13] The aforementioned nucleic acid or composition, wherein the polypeptide into which the modification has been introduced maintains a function of the polypeptide prior to modification.
   [14] The aforementioned nucleic acid or composition, wherein the Fc region into which the modification has been introduced maintains a function of the Fc of IgG.
   [15] The aforementioned nucleic acid or composition, wherein the Fc region is derived from any one of IgG1, 2, 3, and 4.
   [16] A host cell into which the aforementioned nucleic acid or composition has been introduced.
   [17] A polypeptide expressed from the aforementioned nucleic acid.
   [18] A composition comprising a nucleic acid encoding a polypeptide,
wherein the polypeptide comprises an Fc region into which a modification has been introduced,
wherein the modification is at least one of the modifications set forth in Table 2.
   [19] A polypeptide which comprises an Fc region into which a modification has been introduced, wherein the polypeptide associates more readily with a polypeptide having the modification via the Fc region than with a polypeptide comprising an Fc region into which the modification has not been introduced due to at least one of the following actions resulting from the introduced modification: (1) steric complementarity, (2) disulfide linkage, and (3) electrostatic charge.
   [19-1] The aforementioned polypeptide, wherein the modification is a modification in CH3.
   [20] A method for obtaining a polypeptide for which association is controlled, comprising the step of obtaining a nucleic acid encoding the polypeptide; and
the step of expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.
   [20-1] The aforementioned method, wherein the expression is in vivo, ex vivo, or in vitro expression.
   [21] A method for controlling association of a homomer of a polypeptide, comprising:
the step of obtaining a nucleic acid encoding the polypeptide; and
the step of expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.
   [22] A method for promoting the expression of a homomer of a polypeptide, comprising the step of obtaining a nucleic acid encoding the polypeptide; and
the step of expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

Furthermore, the present disclosure relates to the following. The technical features set forth in [1]-[22] above are also incorporated into the following inventions as appropriate.

[101] A polypeptide for which association is controlled, wherein the polypeptide comprises an Fc region into which a modification has been introduced, wherein, because of the modification introduced into the Fc region, the polypeptide associates more readily with a polypeptide having the modification via the Fc region than with a polypeptide into which the modification has not been introduced.
[101-1] The aforementioned polypeptide, wherein the polypeptide associates less readily with a polypeptide into which the modification has not been introduced than with a polypeptide having the modification.
[101-1-1] The aforementioned polypeptide, wherein the polypeptide less readily forms a heterodimer with another polypeptide that does not have the modification.
[101-1-2] The aforementioned polypeptide, which less readily forms a heterodimer than a homodimer.
[101-1-3] The aforementioned polypeptide, wherein the polypeptide does not associate, or associates less readily, with endogenous IgG or an antibody fragment containing an Fc region thereof.
[101-2] The aforementioned polypeptide, wherein the polypeptides having the modification associate with each other.
[101-2-1] The aforementioned polypeptide, wherein the polypeptide having the modification forms a homomer.
[101-2-2] The aforementioned polypeptide, which more readily forms a homodimer than a heterodimer.
[101-2-3] The aforementioned polypeptide, which exhibits a stronger homodimerization-promoting ability than a control (e.g., wild-type IgG).
[101-3] The aforementioned polypeptide, wherein the polypeptide having the modification has an increased ability to form an associated multimer with a polypeptide having the modification as compared to a polypeptide that does not have the modification.
[101-3-1] The aforementioned polypeptide, wherein the association is association between Fc regions.
[101-4] The aforementioned polypeptide, wherein the polypeptide has CH3.
[101-4-1] The aforementioned polypeptide, wherein the association is association in an interface between CH3 regions.
[101-4-2] The aforementioned polypeptide, wherein the modification is a modification in CH3.
[102] The aforementioned polypeptide, wherein the polypeptide associates more readily with a polypeptide having the modification via the Fc region than with a polypeptide into which the modification has not been introduced due to at least one of the following actions resulting from the introduced modification: (1) steric complementarity, (2) disulfide linkage, and (3) electrostatic charge.
[102-1] The aforementioned polypeptide, wherein the steric complementarity results from the presence of a knob and a hole in the Fc region.
[102-1-1] The aforementioned polypeptide, wherein the modification is a modification to introduce a knob and a hole into the Fc region.
[102-1-2] The aforementioned polypeptide, wherein the knob and the hole in the Fc region induce the formation of a homodimer of the first polypeptide.
[102-1-3] The aforementioned polypeptide, wherein the knob and the hole are all introduced into the first polypeptide.
[102-2] The aforementioned polypeptide, wherein the disulfide linkage is formed by substituting one or more amino acids in the Fc region with cysteine (C).
[102-2-1] The aforementioned polypeptide, wherein the modification is a modification to introduce one or more cysteines into the Fc region.
[102-2-2] The aforementioned polypeptide, wherein the modification is substitution of one or more amino acids in the Fc region with cysteine.
[102-2-3] The aforementioned polypeptide, wherein the disulfide linkage is a linkage between the cysteines modified (introduced) in the Fc region.
[102-2-4] The aforementioned polypeptide, which associates more readily with a polypeptide having the modification than a polypeptide into which the modification has not been introduced due to at least the combinations of the actions of (2) disulfide linkage and (3) electrostatic charge.
[102-3] The aforementioned polypeptide, wherein the action of the electrostatic charge results from one or more charged amino acids modified (introduced) in the Fc region.
[102-3-1] The aforementioned polypeptide, wherein the introduced charged amino acid(s) is a positively charged amino acid and/or a negatively charged amino acid.
[102-3-2] The aforementioned polypeptide, wherein at least two amino acids in the Fc region are substituted with a positively charged amino acid and a negatively charged amino acid.
[102-3-3] The aforementioned polypeptide, wherein the positively charged amino acid is selected from lysine (K), arginine (R), and histidine (H), and/or the negatively charged amino acid is selected from aspartic acid (D) and glutamic acid (E).
[102-4] The aforementioned polypeptide, wherein the action is (a combination of) more than one action (two or three actions) selected from (1) steric complementarity, (2) disulfide linkage, and (3) electrostatic charge.
[103] The aforementioned polypeptide, wherein the modification introduced into the Fc region is at least one of the modifications set forth in Table 2.
[104] The aforementioned polypeptide, wherein the modification introduced into the Fc region is at least one of the modifications set forth in Table 4.
[105] The aforementioned polypeptide, wherein the steric complementarity comprises knob-into-hole.
[106] The aforementioned polypeptide, wherein the polypeptide is an antibody.
[107] The aforementioned polypeptide, wherein the Fc region is an Fc region of IgG.
[108] The aforementioned polypeptide, wherein the Fc region is derived from any one of IgG1, 2, 3, and 4.
[201] A method for producing a polypeptide for which association between Fc regions is controlled, comprising:
(a) obtaining a nucleic acid encoding a polypeptide into which a modification has been introduced in the Fc region, wherein the polypeptide associates more readily with a polypeptide having the modification than with a polypeptide into which the modification has not been introduced due to at least one of the following actions: (1) steric complementarity, (2) disulfide linkage, and (3) electrostatic charge;
(b) introducing the nucleic acid into a host cell and culturing the host cell to express the nucleic acid; and
(c) recovering the polypeptide from the culture of the host cell.
   [201-1] The aforementioned method, which further comprises introducing a modification into the Fc region such that the action of (1) steric complementarity, (2) disulfide linkage, or (3) electrostatic charge occurs in the polypeptide.
   [202-2] The aforementioned method, wherein the modification introduced into the Fc region is at least one of the modifications set forth in Table 2 or Table 4.
   [202] A method for producing a polypeptide for which association between Fc regions is controlled, comprising:
(a) modifying a nucleic acid encoding a polypeptide comprising an Fc region such that the polypeptide associates more readily with a polypeptide having the modification than with a polypeptide into which the modification has not been introduced due to at least one of the following actions: (1) steric complementarity, (2) disulfide linkage, and (3) electrostatic charge;
(b) introducing the modified nucleic acid into a host cell and culturing the host cell to express the nucleic acid; and
(c) recovering the polypeptide from the culture of the host cell.
   [203] A method for controlling association between polypeptides comprising an Fc region, comprising modifying the polypeptide such that the polypeptide associates more readily with a polypeptide having the modification than with a polypeptide into which the modification has not been introduced due to at least one of the following actions: (1) steric complementarity, (2) disulfide linkage, and (3) electrostatic charge.
   [204] A method for generating in a living organism a polypeptide for which association between polypeptides comprising an Fc region is controlled, comprising:
(a) preparing a nucleic acid encoding a polypeptide comprising an Fc region such that the polypeptide comprising an Fc region is modified into a polypeptide that associates more readily with a polypeptide having the modification than with a polypeptide into which the modification has not been introduced due to at least one of the following actions: (1) steric complementarity, (2) disulfide linkage, and (3) electrostatic charge; and
(b) introducing the nucleic acid of step (a) into a living organism.
   [204-1] The aforementioned method, which comprises introducing a modification into the Fc region such that the action of (1) steric complementarity, (2) disulfide linkage, or (3) electrostatic charge occurs in the polypeptide.
   [204-2] The aforementioned method, wherein the nucleic acid is included in a vesicle.
   [204-3] The aforementioned method, which comprises contacting the nucleic acid with a vesicle.
   [204-4] The aforementioned method, wherein the vesicle is a lipid nanoparticle (LNP), a virus, an extracellular vesicle (EV), or a liposome.
   [204-5] The aforementioned method, wherein the nucleic acid is an RNA, a DNA, or a vector or plasmid carrying the nucleic acid.
   [204-6] The aforementioned method, wherein the RNA is a mRNA, a genomic RNA, or a circular RNA.
   [204-7] The aforementioned method, wherein the polypeptide comprising an Fc region is an antibody.
   [204-8] The aforementioned method, wherein the polypeptide comprising an Fc region is a single-domain antibody.
   [204-9] The aforementioned method, wherein the polypeptide comprising an Fc region is IgG.
   [204-10] The aforementioned method, wherein the polypeptide comprising an Fc region does not associate with endogenous IgG.
   [204-11] The aforementioned method, wherein the polypeptide comprising an Fc region retains a function of the Fc of wild-type IgG.
   [204-12] The aforementioned method, which comprises administering the nucleic acid to a subject.
   [204-13] The aforementioned method, wherein the subject is a human.
   [204-14] The aforementioned method, wherein the polypeptide comprising an Fc region is a humanized antibody or a human antibody.
   [205] A method for expressing in a cell a polypeptide for which association between polypeptides comprising an Fc region is controlled, comprising:
(a) preparing a nucleic acid encoding a polypeptide comprising an Fc region such that the polypeptide comprising an Fc region is modified into a polypeptide that associates more readily with a polypeptide having the modification than with a polypeptide into which the modification has not been introduced due to at least one of the following actions: (1) steric complementarity, (2) disulfide linkage, and (3) electrostatic charge; and
(b) introducing the nucleic acid of step (a) into a cell.
   [205-1] The aforementioned method, which comprises introducing a modification into the Fc region such that the action of (1) steric complementarity, (2) disulfide linkage, or (3) electrostatic charge occurs in the polypeptide.
   [205-2] The aforementioned method, wherein the nucleic acid is an RNA, a DNA, or a vector or plasmid carrying the nucleic acid.
   [205-3] The aforementioned method, wherein the RNA is a mRNA, a genomic RNA, or a circular RNA.
   [205-4] The aforementioned method, wherein the polypeptide comprising an Fc region is an antibody.
   [205-5] The aforementioned method, wherein the polypeptide comprising an Fc region is a single-domain antibody.
   [205-6] The aforementioned method, wherein the polypeptide comprising an Fc region is IgG.
   [205-7] The aforementioned method, wherein the cell is a human cell.
   [205-8] The aforementioned method, which is an in vivo, ex vivo, or in vitro method.
   [206] A method for expressing in a cell two or more types of polypeptides comprising an Fc region, wherein association between polypeptides of the same type is controlled:
(a) preparing a nucleic acid encoding a polypeptide comprising an Fc region such that a first polypeptide comprising an Fc region is modified into a polypeptide that associates more readily with a polypeptide having the modification than with a polypeptide into which the modification has not been introduced due to at least one of the following actions: (1) steric complementarity, (2) disulfide linkage, and (3) electrostatic charge; and
(b) preparing a nucleic acid encoding a polypeptide comprising an Fc region such that a second polypeptide comprising an Fc region is modified into a polypeptide that associates more readily with a polypeptide having the modification than with a polypeptide into which the modification has not been introduced due to at least one of the following actions: (1) steric complementarity, (2) disulfide linkage, and (3) electrostatic charge; wherein the modification in the second polypeptide is different from the modification in the first polypeptide.
   [206-1] The aforementioned method, wherein two or more types of polypeptides comprising an Fc region are expressed, and polypeptides of the same type are allowed to form a homomer.
   [206-2] The aforementioned method, wherein the modifications in the two or more types of polypeptides to be expressed are different from each other.
   [206-3] The aforementioned method, wherein the combination of the modifications different from each other is a combination shown in Table 7.
   [207] A method for controlling association of two or more types of polypeptides comprising an Fc region, wherein association between polypeptides of the same type is controlled, wherein the two or more types of polypeptides are modified such that they associate more readily with a polypeptide having the modification than with a polypeptide into which the modification has not been introduced due to at least one of the following actions: (1) steric complementarity, (2) disulfide linkage, and (3) electrostatic charge, and further wherein the modifications in the two or more types of polypeptides are different from each other.
   [207-1] The aforementioned method, wherein the combination of the modifications different from each other is a combination shown in Table 7.
   [208] A method for producing a pharmaceutical composition comprising a vesicle including a nucleic acid encoding a polypeptide for which association between Fc regions is controlled, comprising:
(a) obtaining a nucleic acid encoding a polypeptide into which a modification has been introduced in the Fc region, wherein the polypeptide associates more readily with a polypeptide having the modification than with a polypeptide into which the modification has not been introduced due to at least one of the following actions: (1) steric complementarity, (2) disulfide linkage, and (3) electrostatic charge; and
(b) contacting the nucleic acid of step (a) with a vesicle.
   [208-1] The aforementioned method, wherein the vesicle is a lipid nanoparticle (LNP), a virus, an extracellular vesicle (EV), or a liposome.
   [208-2] The aforementioned method, wherein a pharmaceutically acceptable carrier is further contacted.
   [208-3] The aforementioned method, wherein the nucleic acid is an RNA, a DNA, or a vector or plasmid carrying the nucleic acid.
   [301] A pharmaceutical composition comprising the aforementioned nucleic acid or the aforementioned polypeptide, and a pharmaceutically acceptable carrier.
   [301-1] A pharmaceutical composition comprising a nucleic acid encoding a polypeptide comprising an Fc region for which association is controlled.
   [301-2] The aforementioned pharmaceutical composition, which comprises the nucleic acid included (encapsulated) in a vesicle.
   [301-3] A pharmaceutical composition comprising a nucleic acid encoding a polypeptide comprising an Fc region for which association is controlled, and a vesicle.
   [301-4] The aforementioned pharmaceutical composition, wherein the vesicle is a lipid nanoparticle (LNP), a virus, an extracellular vesicle (EV), or a liposome.
   [301-5] The aforementioned pharmaceutical composition, wherein the nucleic acid is an RNA, a DNA, or a vector or plasmid carrying the nucleic acid.
   [301-6] The aforementioned pharmaceutical composition, which is for generating in a living organism a polypeptide for which association between Fc regions is controlled.
   [301-7] The aforementioned pharmaceutical composition, which is for expressing in a cell a polypeptide for which association between Fc regions is controlled.

In one embodiment, the present disclosure relates to the following:

### Embodiment 1

A nucleic acid encoding a first polypeptide,
wherein the first polypeptide comprises an Fc region into which a modification has been introduced,
wherein, because of the modification introduced into the Fc region, the first polypeptide associates more readily with a first polypeptide having the modification via the Fc region than with a first polypeptide comprising an Fc region into which the modification has not been introduced.

### Embodiment 2

The nucleic acid of Embodiment 1, wherein the first polypeptide associates more readily with a first polypeptide having the modification via the Fc region than with a first polypeptide comprising an Fc region into which the modification has not been introduced due to at least one of the following actions resulting from the introduced modification: (1) steric complementarity, (2) disulfide linkage, and (3) electrostatic charge.

### Embodiment 3

A composition comprising:
the nucleic acid of present disclosure 1; and
a nucleic acid encoding a second polypeptide,
wherein the second polypeptide comprises an Fc region into which a modification has been introduced,
wherein, because of the modification introduced into the Fc region, the second polypeptide associates more readily with a second polypeptide having the modification via the Fc region than with a second polypeptide comprising an Fc region into which the modification has not been introduced.

### Embodiment 4

A composition comprising:
the nucleic acid of Embodiment 1;
a nucleic acid encoding a second polypeptide; and
a nucleic acid encoding a third polypeptide,
wherein the second polypeptide associates more readily with the third polypeptide than with the second polypeptide.

### Embodiment 5

A composition comprising:
the nucleic acid of Embodiment 2; and
a nucleic acid encoding a second polypeptide,
wherein the second polypeptide does not have the modification the first polypeptide has.

### Embodiment 6

The composition of Embodiment 3, wherein the modification introduced into the first polypeptide is different from the modification introduced into the second polypeptide.

### Embodiment 7

The composition of Embodiment 3, wherein the second polypeptide associates more readily with a second polypeptide having the modification via the Fc region than with a second polypeptide comprising an Fc region into which the modification has not been introduced due to at least one of the following actions resulting from the introduced modification: (1) steric complementarity, (2) disulfide linkage, and (3) electrostatic charge.

### Embodiment 8

The nucleic acid of Embodiment 1, wherein the modification introduced into the Fc region is at least one of the modifications set forth in Table 2.

### Embodiment 9

The nucleic acid of Embodiment 1, wherein the modification introduced into the Fc region is at least one of the modifications set forth in Table 4.

### Embodiment 10

The nucleic acid of Embodiment 6, wherein the modification introduced into the first polypeptide and the modification introduced into the second polypeptide are at least one of the combinations of modifications set forth in Table 6.

### Embodiment 11

The nucleic acid of Embodiment 1, wherein the polypeptide is an antibody.

### Embodiment 12

The nucleic acid of Embodiment 1, wherein the Fc region is an Fc region of IgG.

### Embodiment 13

The nucleic acid of Embodiment 1, wherein the polypeptide into which the modification has been introduced retains a function of the polypeptide prior to modification.

### Embodiment 14

The nucleic acid of Embodiment 12, wherein the Fc region into which the modification has been introduced retains a function of an Fc of IgG.

### Embodiment 15

The nucleic acid of Embodiment 1, wherein the Fc region is derived from any one of IgG1, 2, 3, and 4.

### Embodiment 16

A host cell into which the nucleic acid of any one of Embodiments 1, 2, and 8-14 or the composition of any one of Embodiments 3-7 has been introduced.

### Embodiment 17

A polypeptide expressed from the nucleic acid of Embodiment 1.

### Embodiment 18

A composition comprising a nucleic acid encoding a polypeptide, wherein the polypeptide comprises an Fc region into which a modification has been introduced, wherein the modification is at least one of the modifications set forth in Table 2.

### Embodiment 19

A polypeptide which comprises an Fc region into which a modification has been introduced, wherein the polypeptide associates more readily with a polypeptide having the modification via the Fc region than with a polypeptide comprising an Fc region into which the modification has not been introduced due to at least one of the following actions resulting from the introduced modification: (1) steric complementarity, (2) disulfide linkage, and (3) electrostatic charge.

### Embodiment 20

A method for obtaining a polypeptide for which association is controlled, comprising: obtaining a nucleic acid encoding the polypeptide; and
expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

### Embodiment 21

A method for controlling association of a homomer of a polypeptide, comprising: obtaining a nucleic acid encoding the polypeptide; and
expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

### Embodiment 22

A method for promoting expression of a homomer of a polypeptide, comprising: obtaining a nucleic acid encoding the polypeptide; and
expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

### Embodiment 23

The composition of Embodiment 4, wherein the second polypeptide comprises an Fc region into which a modification has been introduced,
wherein, because of the modification introduced into the Fc region, the second polypeptide associates more readily with the third polypeptide via the Fc region than with the second polypeptide.

### Embodiment 24

The composition of Embodiment 1, wherein the Fc region into which the modification has been introduced comprises an amino acid modification at one combination or two or more combinations of positions selected from the combinations of positions shown in (a) to (d) below according to EU numbering:
(a) positions 394 and 405;
(b) positions 366, 368, and 407;
(c) positions 347, 360, 399, 405 and 409; and
(d) positions 356, 392, 399, and 439.

### Embodiment 25

The composition of Embodiment 24, wherein the Fc region into which the modification has been introduced comprises at least one amino acid selected from the group consisting of:
(a) W, F, or Y at position 394, and
   A, S, T, C, G, or V at position 405;
(b) W, Y, or F at position 366,
   A, S, T, C, V, or G at position 368, and
   V, L, I, M, A, S, T, C, N, or Q at position 407;
(c) R, K, Y, or H at position 347,
   E or D at position 360,
   V, L, I, M, S, T, C, H, A, N, Q, or G at position 399,
   T, A, V, S, C, N, D, or G at position 405, and
   W, F, Y, or H at position 409; and
(d) K or R at position 356,
   D or E at position 392,
   K or R at position 399, and
   E or D at position 439,
according to EU numbering.

### Embodiment 26

The composition of Embodiment 24, wherein the Fc region into which the modification has been introduced comprises at least one amino acid selected from the group consisting of:
(a) W or F at position 394, and
   A, S, T, or G at position 405;
(b) W, Y, or F at position 366,
   A, T, C, V, or G at position 368, and
   V, L, I, M, A, or C at position 407;
(c) R, K, Y, or H at position 347
   E or D at position 360
   V, L, I, M, S, T, C, H, A, N, or G at position 399,
   T, A, or V at position 405, and
   W, F, or Y at position 409; and
(d) K at position 356,
   D at position 392,
   K at position 399, and
   E at position 439,
according to EU numbering.

### Embodiment 27

The nucleic acid of Embodiment 1, wherein the modification introduced into the Fc region is at least one of the modifications set forth in Table 15.

### Embodiment 28

The nucleic acid of Embodiment 1, wherein the modification introduced into the Fc region is at least one of the modifications set forth in Table 17.

In one embodiment, the present disclosure relates to the following:

### Embodiment 29

The composition of Embodiment 1, wherein the Fc region into which the modification has been introduced comprises amino acid modifications at one combination or two or more combinations of positions selected from the combinations of positions shown in (a) to (d) below according to EU numbering:
(a) positions 345, 347, 360, 366, 399, 407, and 409;
(b) positions 356, 399, 409, and 439;
(c) positions 356, 392, 399, and 409; and
(d) positions 392, 399, and 409.

### Embodiment 30

The composition of Embodiment 29, wherein the Fc region into which the modification has been introduced comprises at least one amino acid selected from the group consisting of:
(a) R or E at position 345,
   R or K at position 347,
   D or E at position 360,
   V at position 366,
   M, Q, N, H, I, F, Y, T, S, V, or L at position 399,
   A at position 407, and
   V, Q, N, H, L, I, F, Y, T, or S at position 409;
(b) K, R, or H at position 356,
   K, R, or H at position 399,
   E or D at position 409, and
   E or D at position 439,
(c) K, R, or H at position 356,
   D or E at position 392,
   K, R, or H at position 399, and
   D or E at position 409; and
(d) D or E at position 392,
   K or R at position 399, and
   D or E at position 409,
according to EU numbering.

### BRIEF DESCRIPTION OF DRAWINGS

FIG. 1 shows the concept of the control of CH3 interface association which promotes homodimer formation of heavy chains. (1) Amino acid modifications with charges that cause modified CH3 regions to have an attractive force with each other but a repulsive force with wild-type CH3. (2) Amino acid modifications for producing modified CH3 with a protuberance and a cavity, where modified CH3 regions can bind with each other but cannot associate with wild-type CH3 due to steric hindrance. (3) Amino acid modifications that allow multiple artificial disulfide bonds to be formed between modified CH3 regions. Association with wild-type CH3 is suppressed.

FIG. 2 shows a method of screening for heavy chain homodimerization-promoting modifications by SEC analysis.

FIG. 3 (FIG. 3-1 to FIG. 3-47) shows the evaluation of heavy chain association of antibodies by CIEX analysis. In FIG. 3-1, one antibody without CH3 modification is analyzed.

In FIG. 3-2, one antibody with CH3 modification is analyzed.

In FIG. 3-3, one antibody without CH3 modification is analyzed.

In FIG. 3-4, one antibody with CH3 modification is analyzed.

In FIG. 3-5, one antibody without CH3 modification is analyzed.

In FIG. 3-6, one antibody with CH3 modification is analyzed.

In FIG. 3-7, one antibody without CH3 modification is analyzed.

In FIG. 3-8, one antibody with CH3 modification is analyzed.

FIG. 3-9 shows the result of analysis in coexpression of two antibodies without CH3 modification. Each peak position was identified and assigned based on the elution position observed when expressing each one antibody.

FIG. 3-10 shows the result of analysis in coexpression of two antibodies with CH3 modification. Each peak position was identified and assigned based on the eluted position observed when expressing each one antibody.

FIG. 3-11 shows the result of analysis in coexpression of two antibodies without CH3 modification. Each peak position was identified and assigned based on the eluted position observed when expressing each one antibody.

FIG. 3-12 shows the result of analysis in coexpression of two antibodies with CH3 modification. Each peak position was identified and assigned based on the elution position observed when expressing each one antibody.

FIG. 3-13 shows the result of analysis in coexpression of two antibodies without CH3 modification. Each peak position was identified and assigned based on the elution position observed when expressing each one antibody.

FIG. 3-14 shows the result of analysis in coexpression of two antibodies with CH3 modification. Each peak position was identified and assigned based on the elution position observed when expressing each one antibody.

FIG. 3-15 shows the result of analysis in coexpression of two antibodies without CH3 modification. Each peak position was identified and assigned based on the elution position observed when expressing each one antibody.

FIG. 3-16 shows the result of analysis in coexpression of two antibodies with CH3 modification. Each peak position was identified and assigned based on the elution position observed when expressing each one antibody.

FIG. 3-17 shows the result of analysis in coexpression of two antibodies without CH3 modification. Each peak position was identified and assigned based on the elution position observed when expressing each one antibody.

FIG. 3-18 shows the result of analysis in coexpression of two antibodies with CH3 modification. Each peak position was identified and assigned based on the elution position observed when expressing each one antibody.

FIG. 3-19 shows the result of analysis in coexpression of two antibodies without CH3 modification. Each peak position was identified and assigned based on the elution position observed when expressing each one antibody.

FIG. 3-20 shows the result of analysis in coexpression of two antibodies with CH3 modification. Each peak position was identified and assigned based on the elution position observed when expressing each one antibody.

FIG. 3-21 shows the result of analysis in coexpression of three antibodies without CH3 modification. Each peak was assigned with reference to the peak positions observed when expressing one antibody or coexpressing two antibodies.

FIG. 3-22 shows the result of analysis in coexpression of three antibodies with CH3 modification. Each peak was assigned with reference to the peak positions observed when expressing one antibody or coexpressing two antibodies.

FIG. 3-23 shows the result of analysis in coexpression of three antibodies without CH3 modification. Each peak was assigned with reference to the peak positions observed when expressing one antibody or coexpressing two antibodies.

FIG. 3-24 shows the result of analysis in coexpression of three antibodies with CH3 modification. Each peak was assigned with reference to the peak positions observed when expressing one antibody or coexpressing two antibodies.

FIG. 3-25 shows the result of analysis in coexpression of three antibodies without CH3 modification. Each peak was assigned with reference to the peak positions observed when expressing one antibody or coexpressing two antibodies.

FIG. 3-26 shows the result of analysis in coexpression of three antibodies with CH3 modification. Each peak was assigned with reference to the peak positions observed when expressing one antibody or coexpressing two antibodies.

FIG. 3-27 shows the result of analysis in coexpression of four antibodies without CH3 modification. Each peak was assigned with reference to the peak positions observed when expressing one antibody or coexpressing two antibodies.

FIG. 3-28 shows the result of analysis in coexpression of four antibodies with CH3 modification. Each peak was assigned with reference to the peak positions observed when expressing one antibody or coexpressing two antibodies.

In FIG. 3-29, one antibody with CH3 modification is analyzed.

FIG. 3-30 shows the result of analysis in coexpression of two antibodies with CH3 modification. Each peak position was identified and assigned based on the elution position observed when expressing each one antibody.

FIG. 3-31 shows the result of analysis in coexpression of two antibodies with CH3 modification. Each peak position was identified and assigned based on the elution position observed when expressing each one antibody.

FIG. 3-32 shows the result of analysis in coexpression of two antibodies with CH3 modification. Each peak position was identified and assigned based on the elution position observed when expressing each one antibody.

FIG. 3-33 shows the result of analysis in coexpression of three antibodies with CH3 modification. Each peak was assigned with reference to the peak positions observed when expressing one antibody or coexpressing two antibodies.

FIG. 3-34 shows the result of analysis in coexpression of three antibodies with CH3 modification. Each peak was assigned with reference to the peak positions observed when expressing one antibody or coexpressing two antibodies.

FIG. 3-35 shows the result of analysis in coexpression of four antibodies with CH3 modification. Each peak was assigned with reference to the peak positions observed when expressing one antibody or coexpressing two antibodies.

In FIG. 3-36, one antibody with CH3 modification is analyzed.

FIG. 3-37 shows the result of analysis in coexpression of two antibodies with CH3 modification. Each peak position was identified and assigned based on the elution position observed when expressing each one antibody.

FIG. 3-38 shows the result of analysis in coexpression of two antibodies with CH3 modification. Each peak position was identified and assigned based on the elution position observed when expressing each one antibody.

FIG. 3-39 shows the result of analysis in coexpression of two antibodies with CH3 modification. Each peak position was identified and assigned based on the elution position observed when expressing each one antibody.

FIG. 3-40 shows the result of analysis in coexpression of three antibodies with CH3 modification. Each peak was assigned with reference to the peak positions observed when expressing one antibody or coexpressing two antibodies.

FIG. 3-41 shows the result of analysis in coexpression of three antibodies with CH3 modification. Each peak was assigned with reference to the peak positions observed when expressing one antibody or coexpressing two antibodies.

FIG. 3-42 shows the result of analysis in coexpression of three antibodies with CH3 modification. Each peak was assigned with reference to the peak positions observed when expressing one antibody or coexpressing two antibodies.

FIG. 3-43 shows the result of analysis in coexpression of four antibodies with CH3 modification. Each peak was assigned with reference to the peak positions observed when expressing one antibody or coexpressing two antibodies.

FIG. 3-44 shows the result of analysis in coexpression of two antibodies with CH3 modification. Each peak position was identified and assigned based on the elution position observed when expressing each one antibody.

FIG. 3-45 shows the result of analysis in coexpression of three antibodies with CH3 modification. Each peak was assigned with reference to the peak positions observed when expressing one antibody or coexpressing two antibodies.

FIG. 3-46 shows the result of analysis in coexpression of three antibodies with CH3 modification. Each peak was assigned with reference to the peak positions observed when expressing one antibody or coexpressing two antibodies.

FIG. 3-47 shows the result of analysis in coexpression of four antibodies with CH3 modification. Each peak was assigned with reference to the peak positions observed when expressing one antibody or coexpressing two antibodies.

FIG. 4 shows the evaluation of ECM binding of CH3-modified antibodies. Non-specific binding of each antibody into which modifications for promoting heavy chain homodimerization were introduced was evaluated.

FIG. 5 (FIG. 5-1 to FIG. 5-14) shows the evaluation of heavy chain association of two antibodies by SEC analysis. Since there were multimeric components resulting from amino acid modification or antibody format, comparison was performed focusing on the heavy chain heteromultimer component indicated by the arrow in the figure. The elution position of the heteromultimer component in the chromatogram was identified by comparison with the elution position of each heavy chain homodimer reference sample. The analysis results of the reference samples are also shown in the figure. In FIG. 5-1, the elution positions of reference antibodies without CH3 modification, and the ability of heavy chain homodimerization under coexpression of full-length heavy chains and Fc fragments, are analyzed.

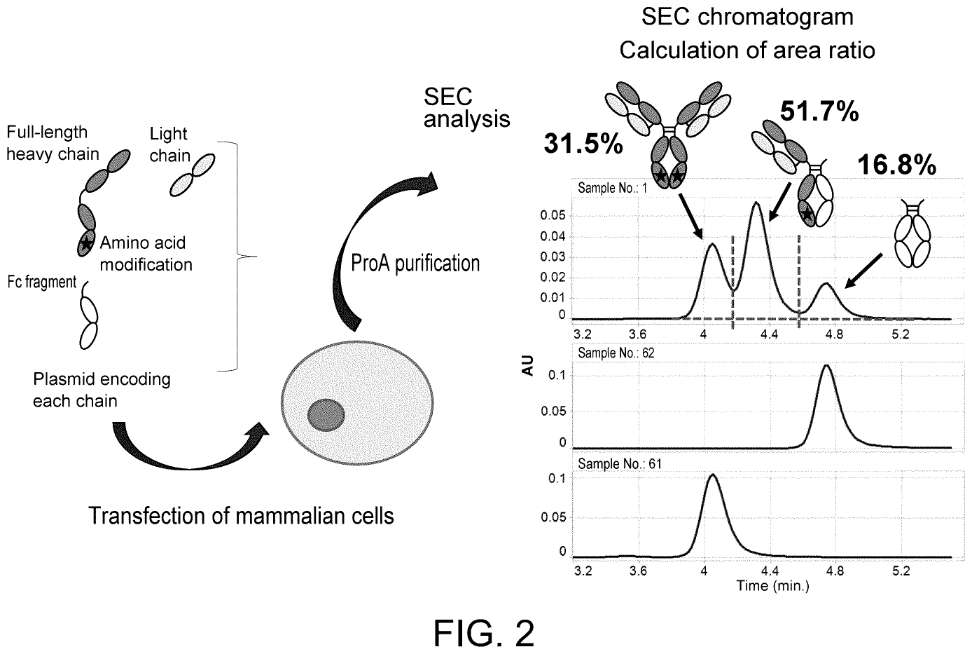

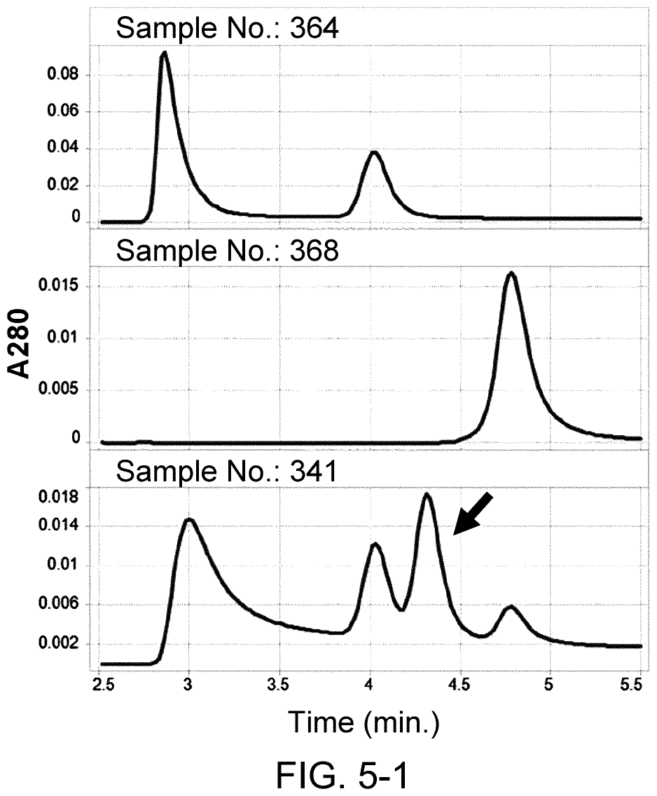

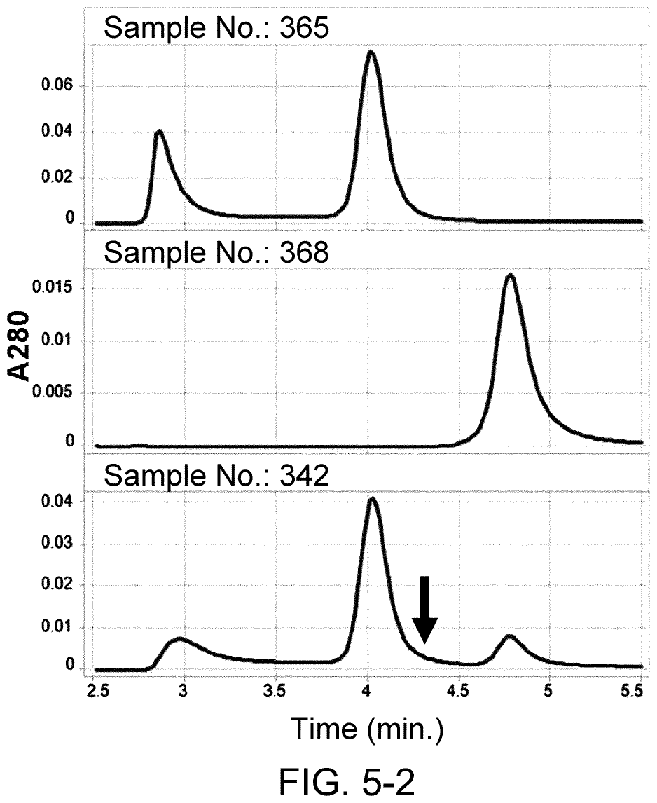

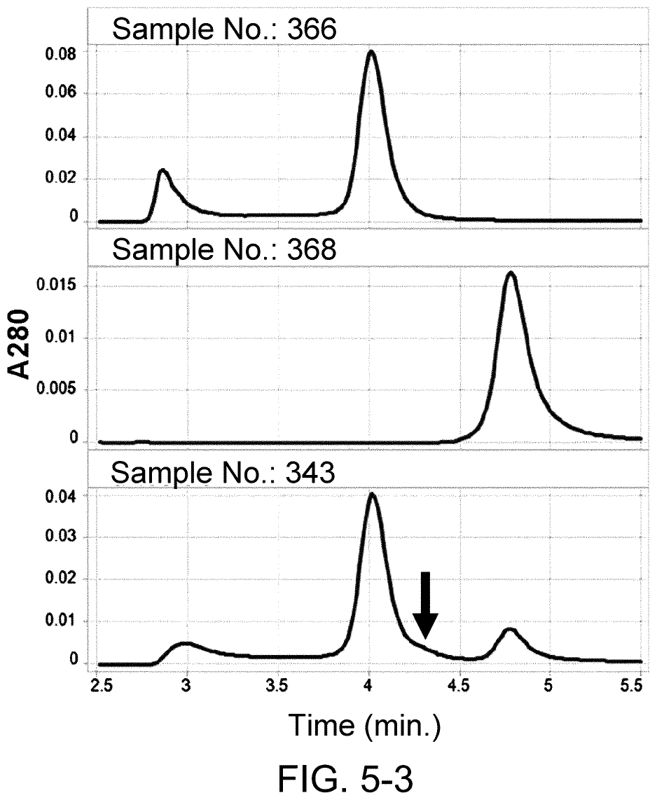

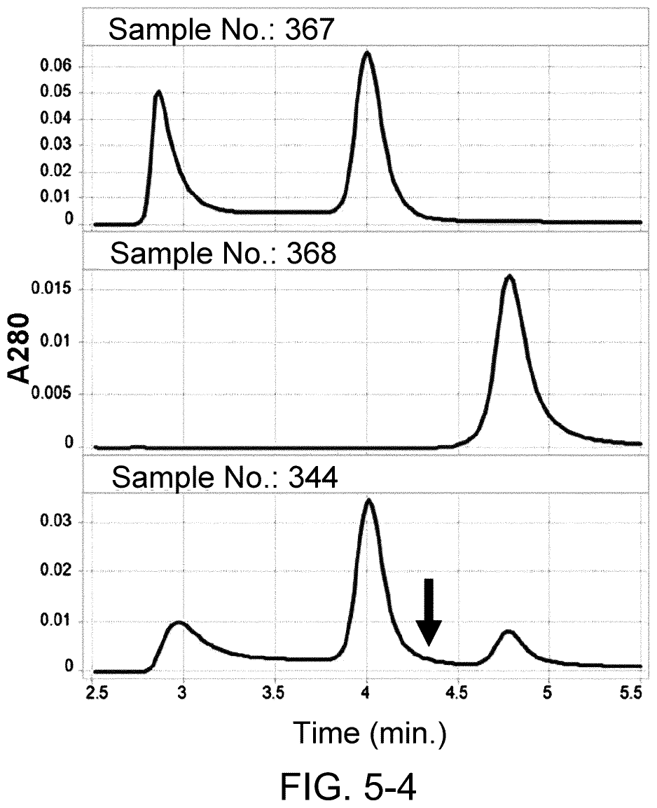

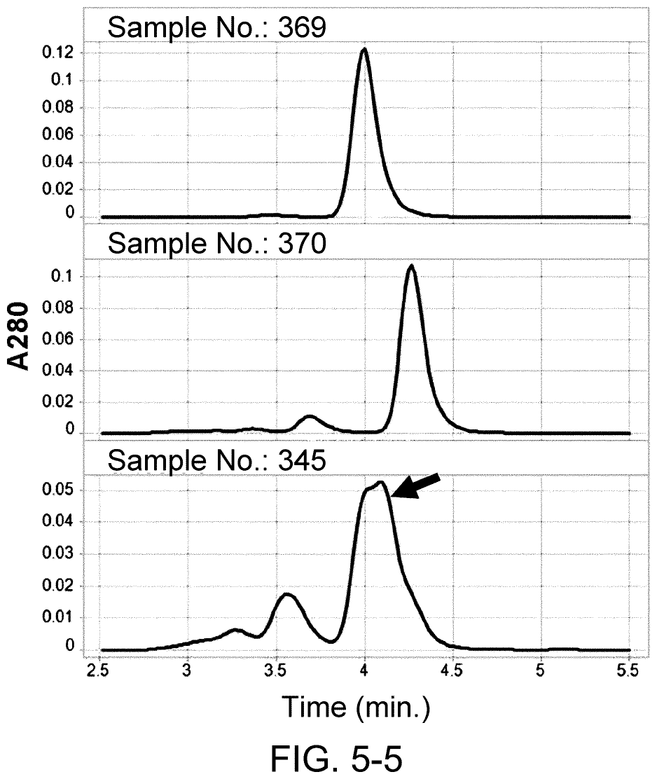

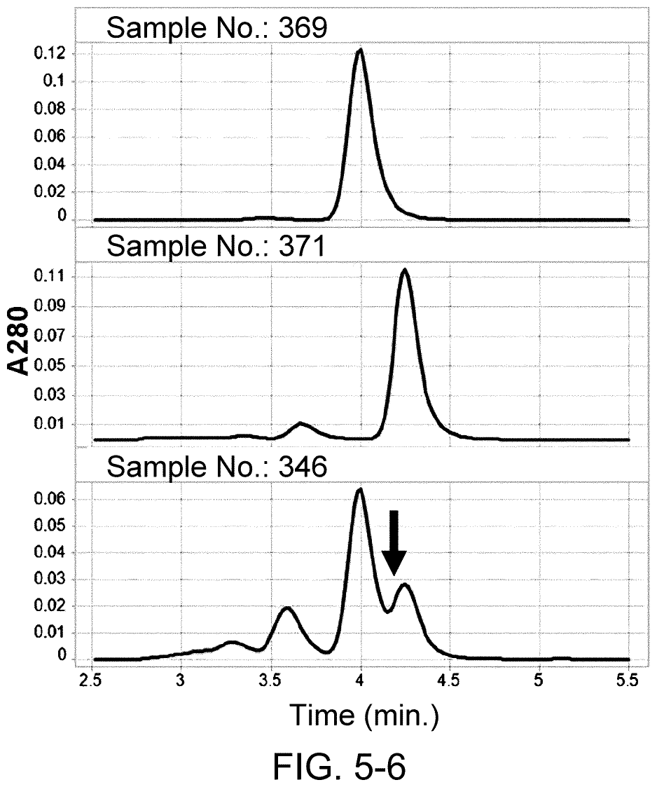

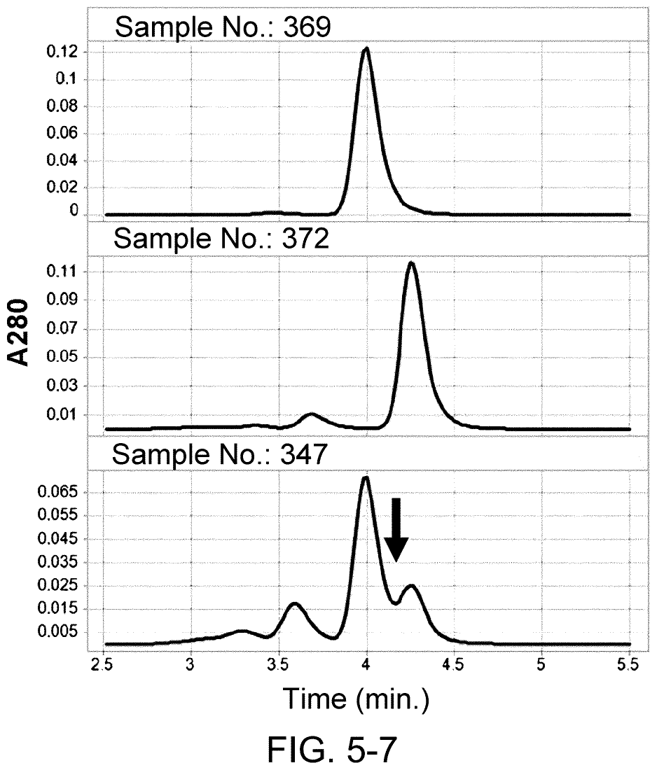

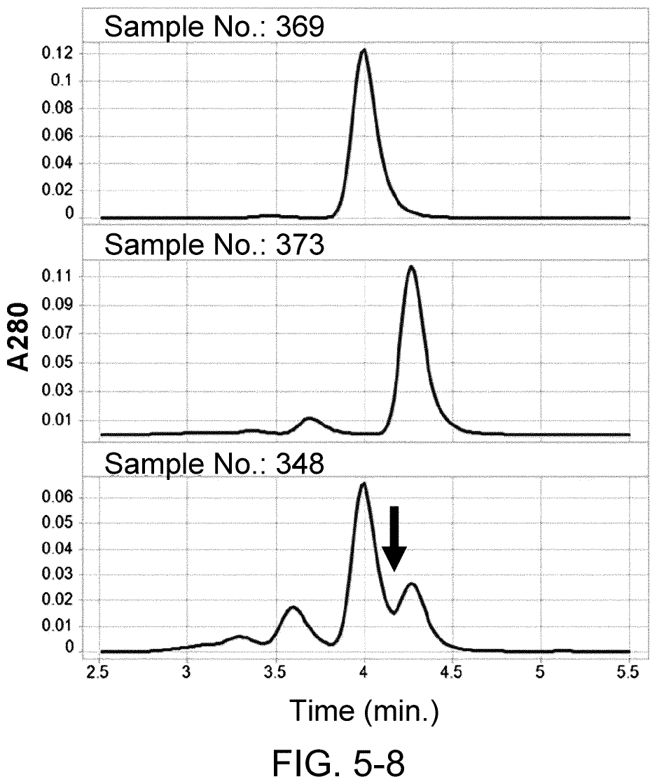

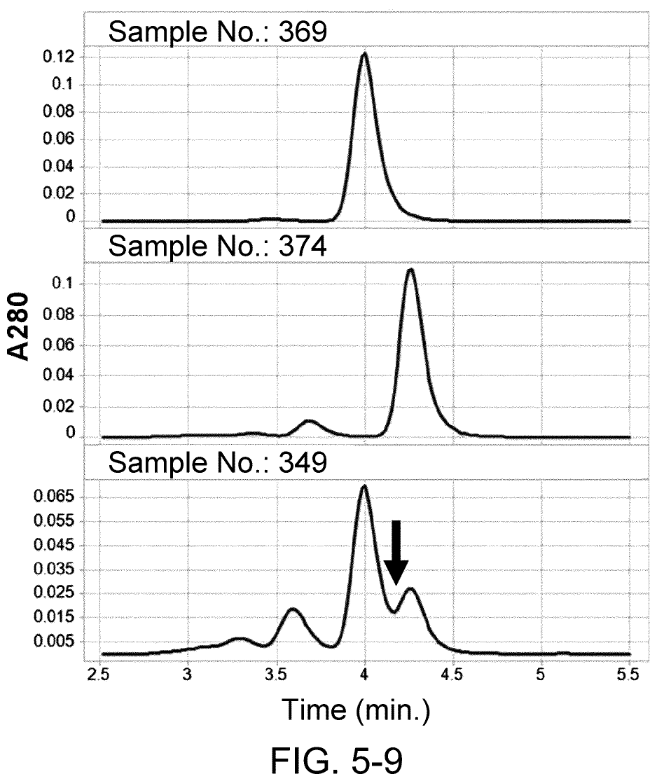

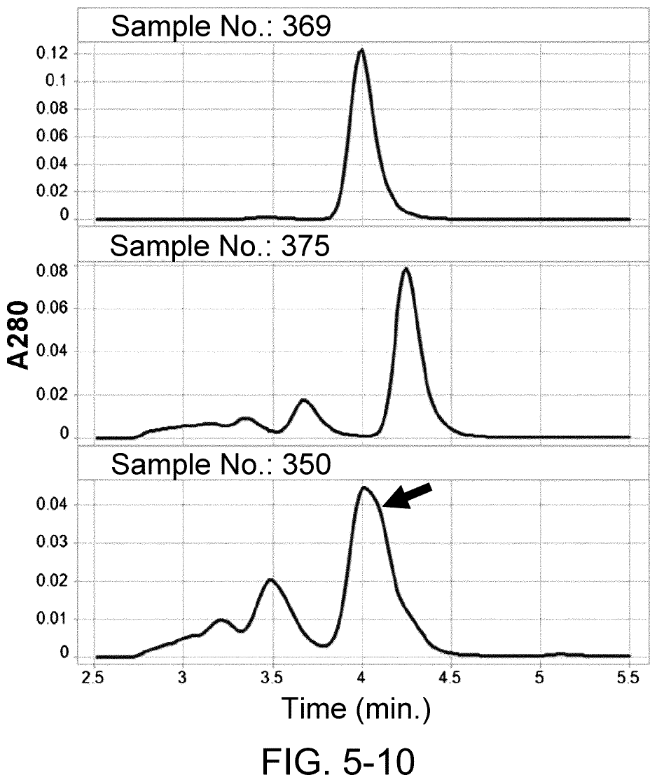

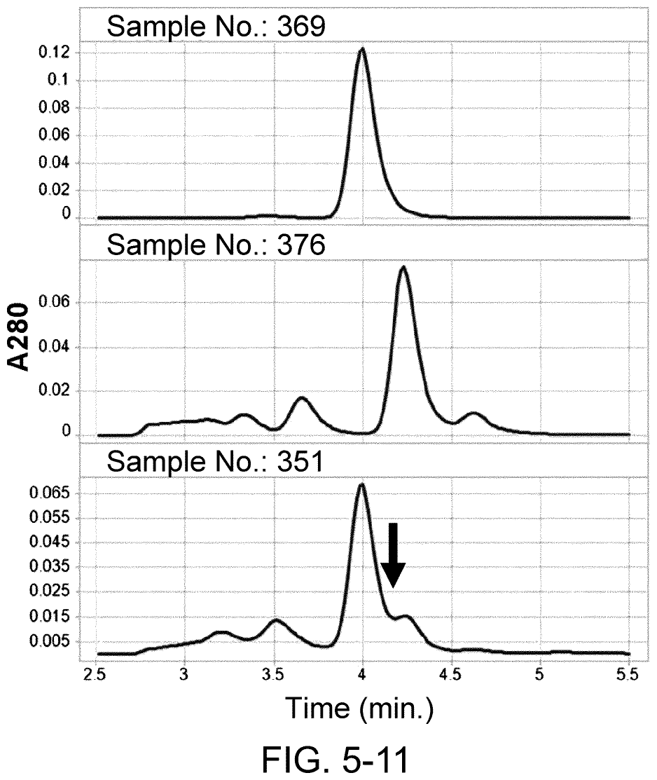

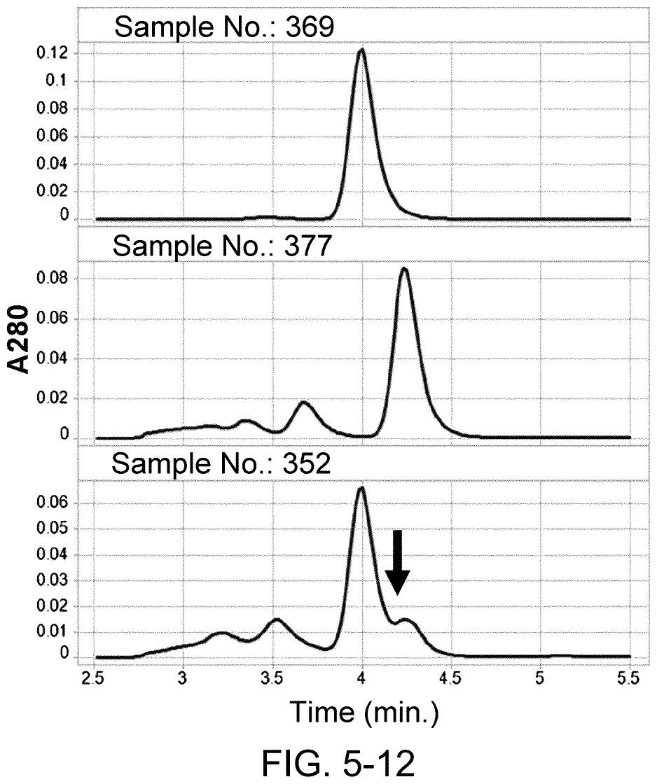

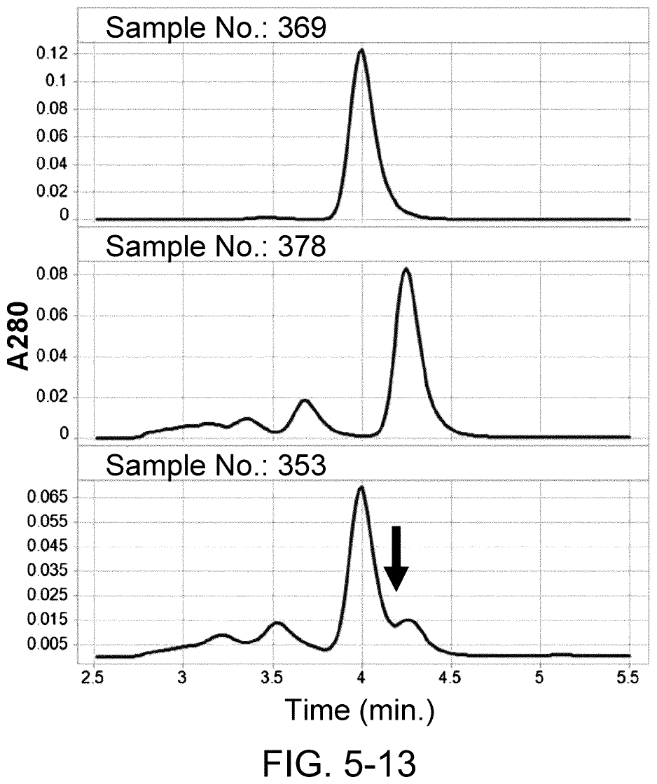

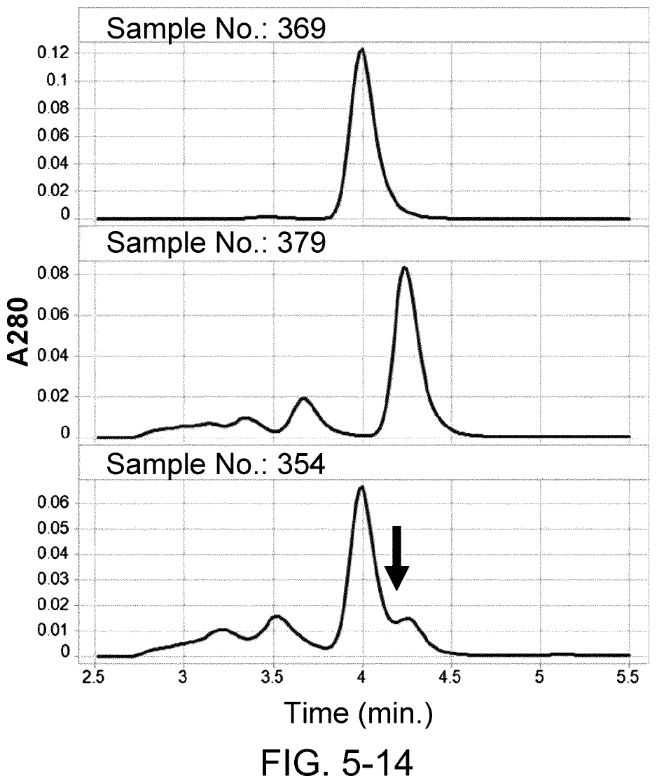

In FIG. 5-2, the elution positions of reference antibodies with CH3 modification, and the ability of heavy chain homodimerization under coexpression of full-length heavy chains and Fc fragments, are analyzed.

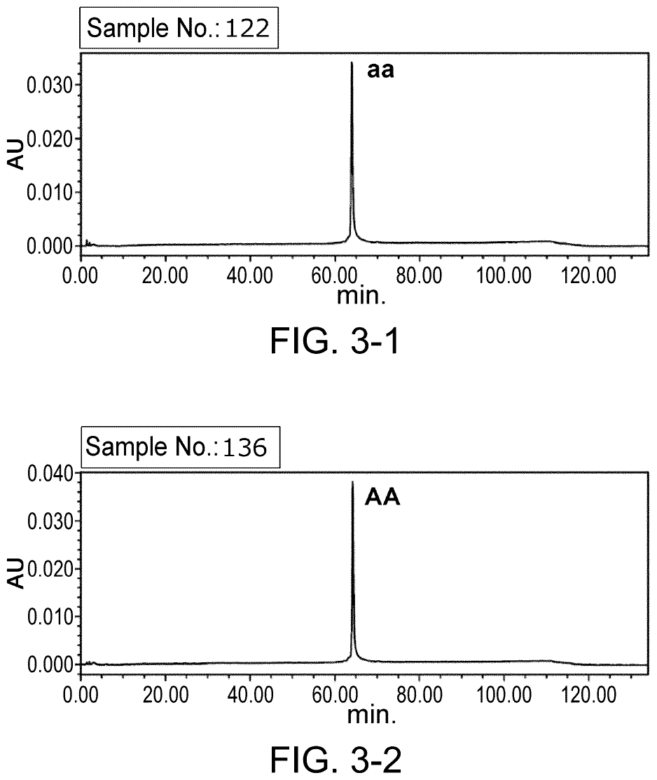

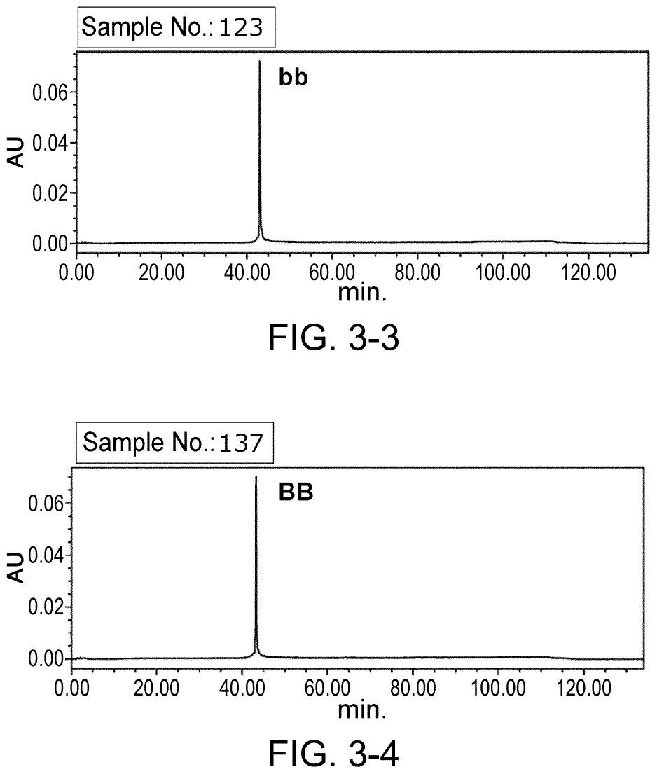

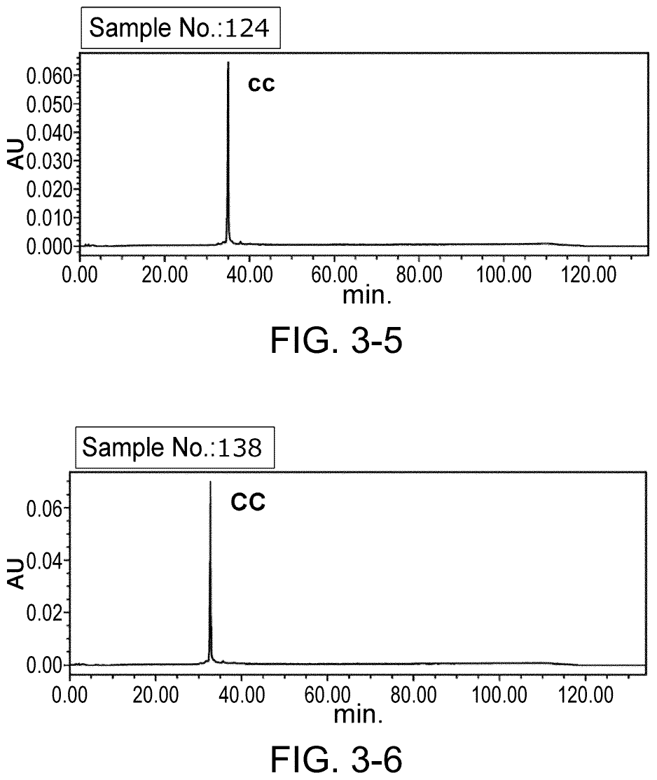

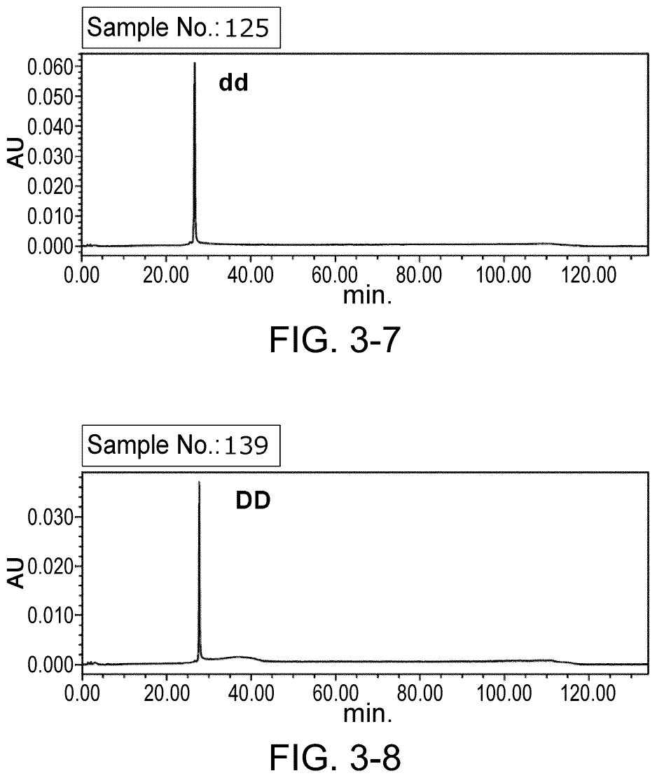

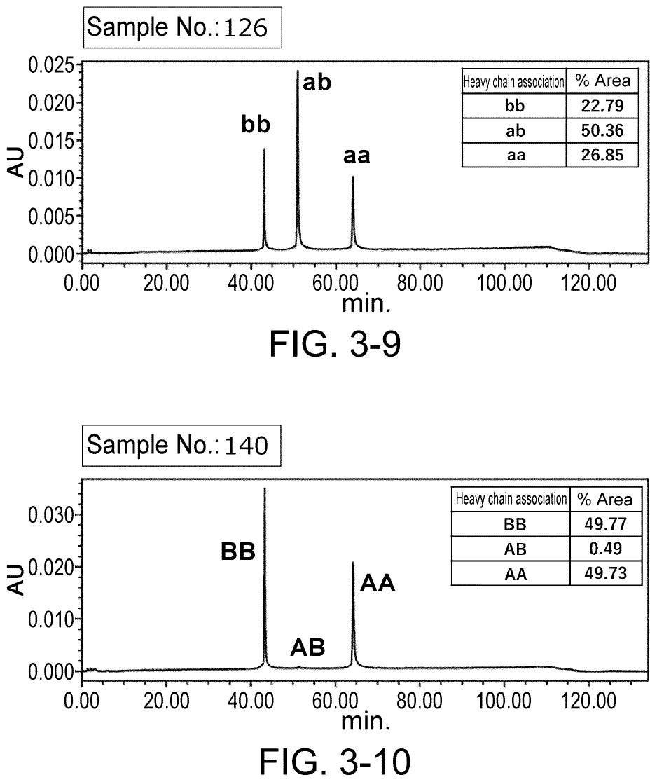

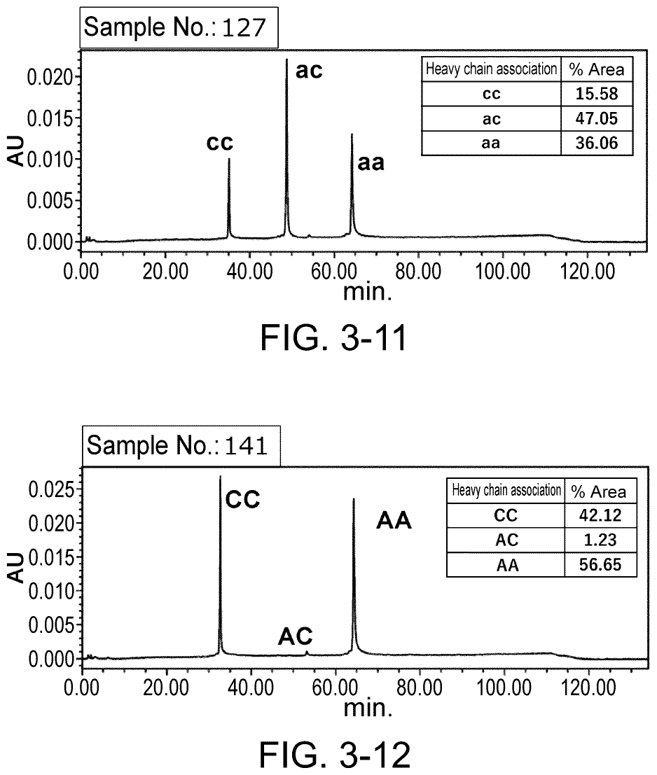

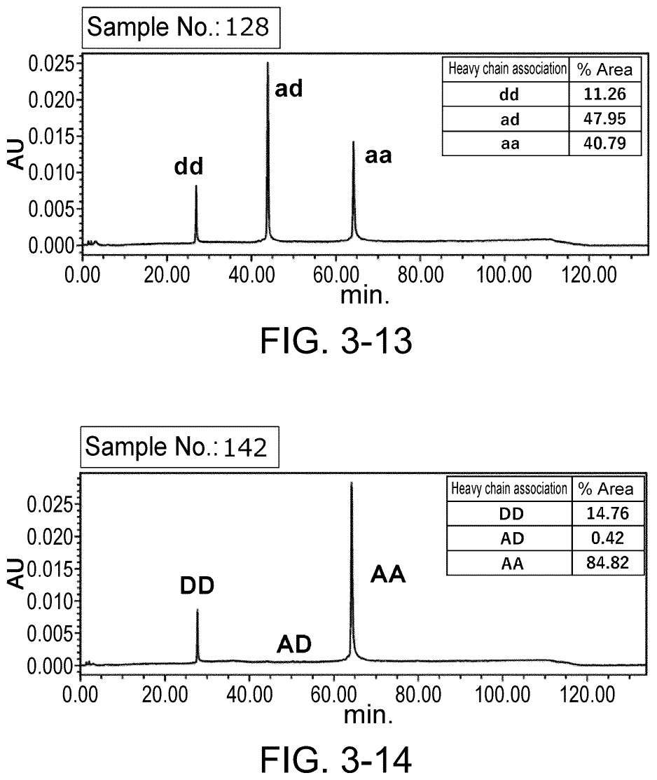

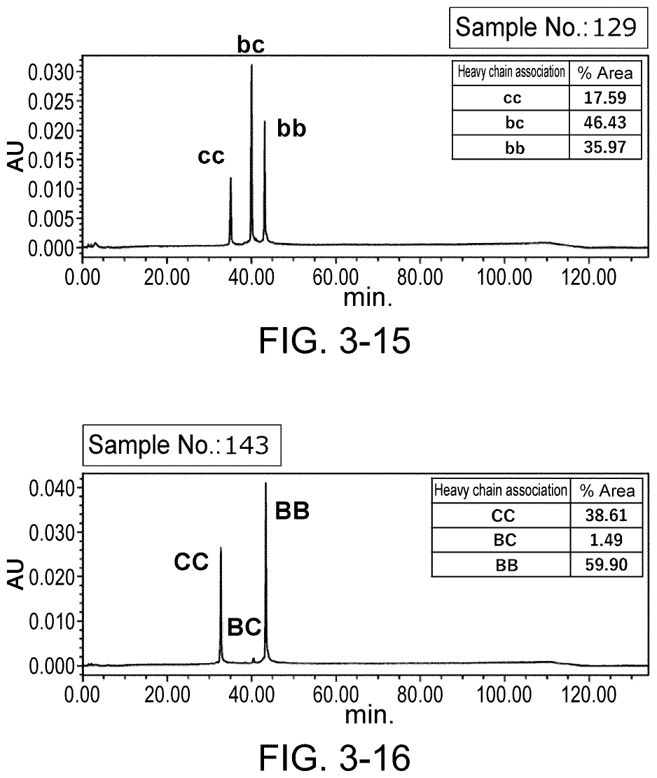

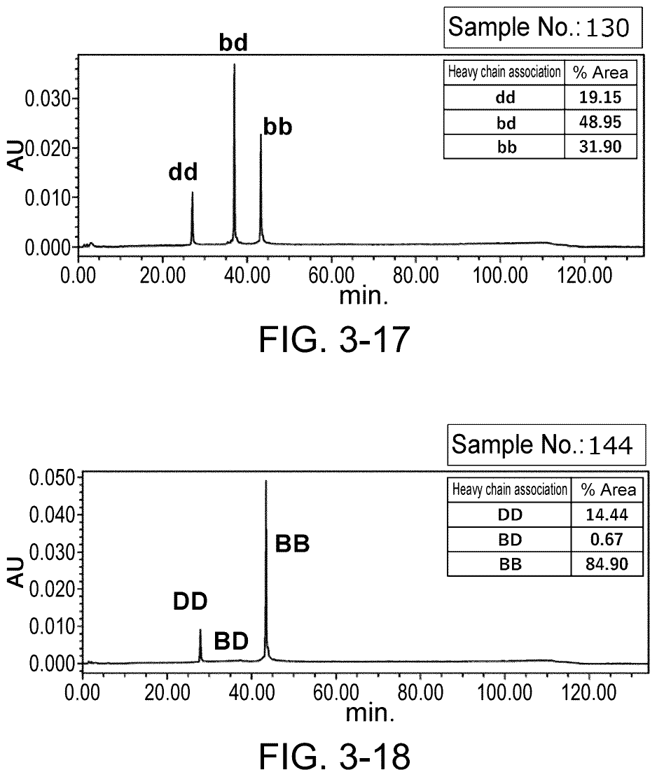

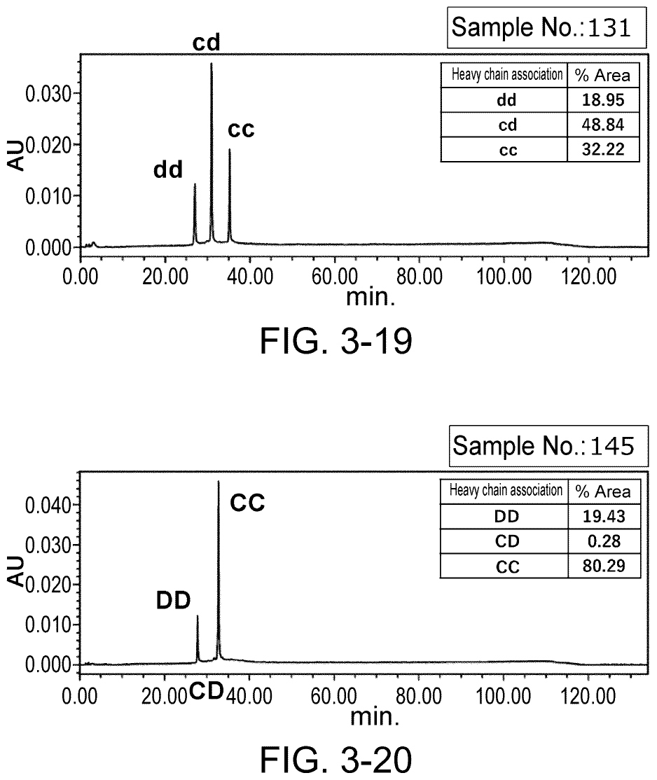

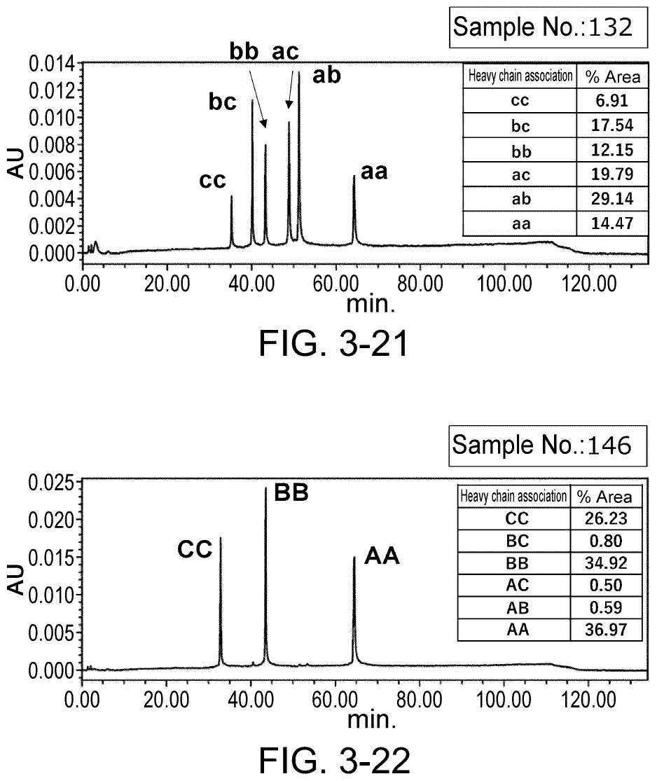

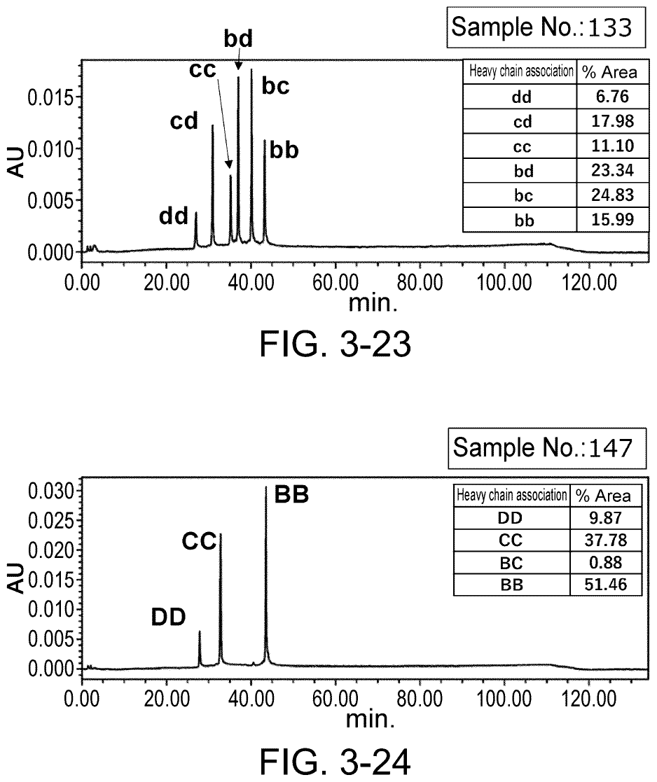

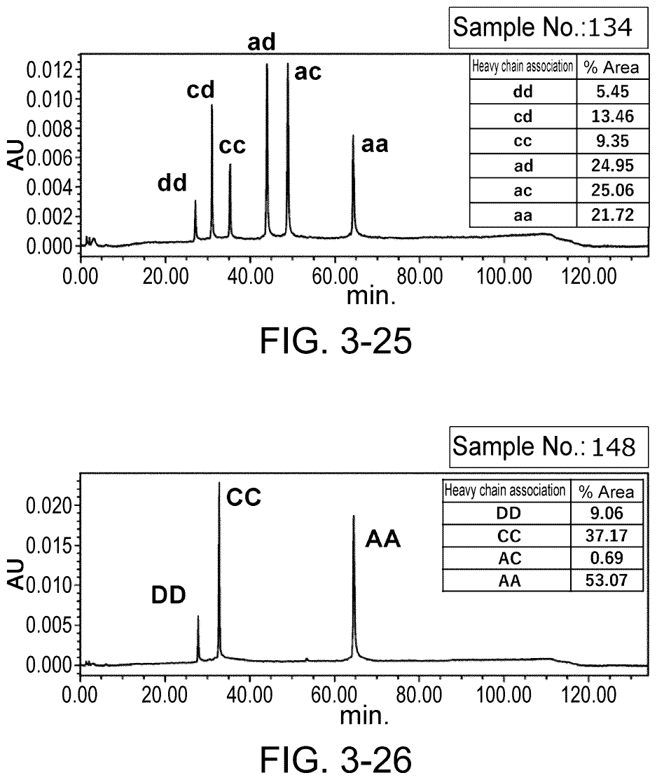

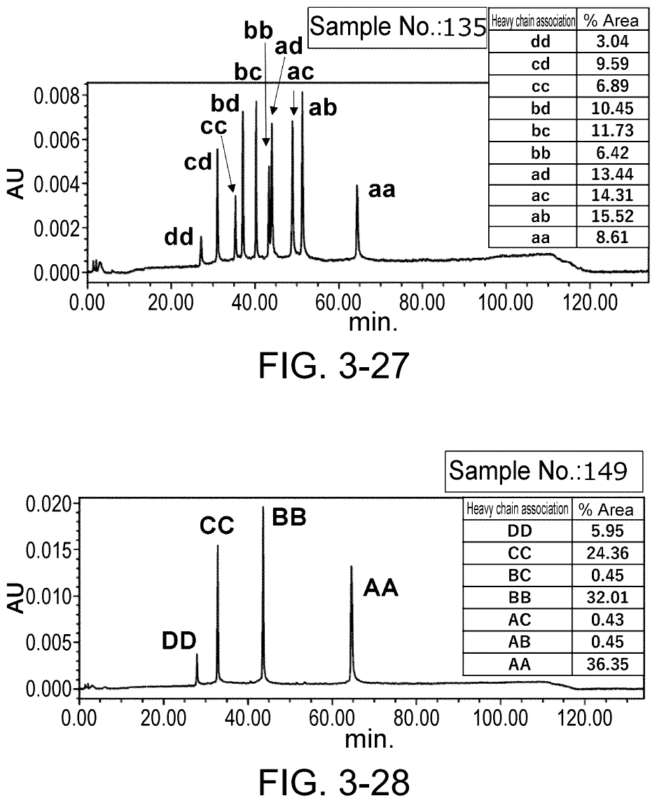

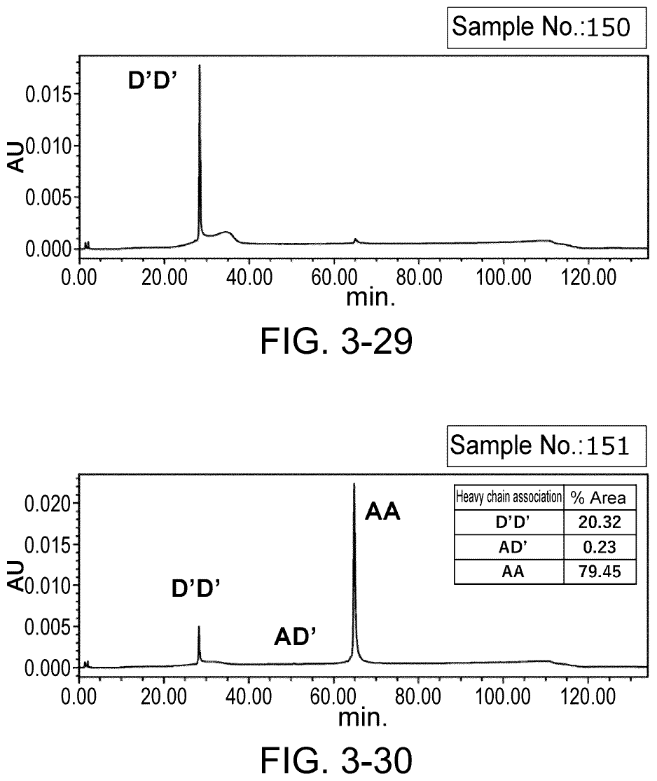

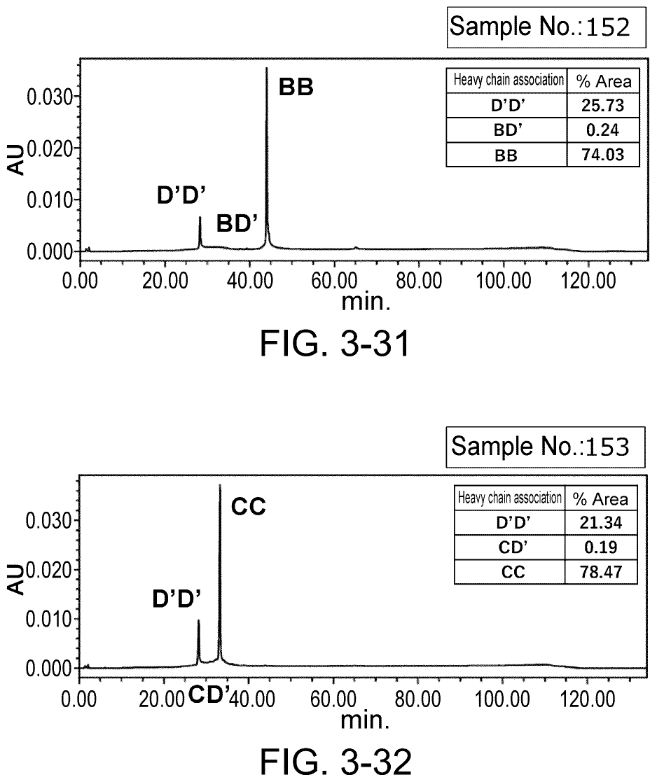

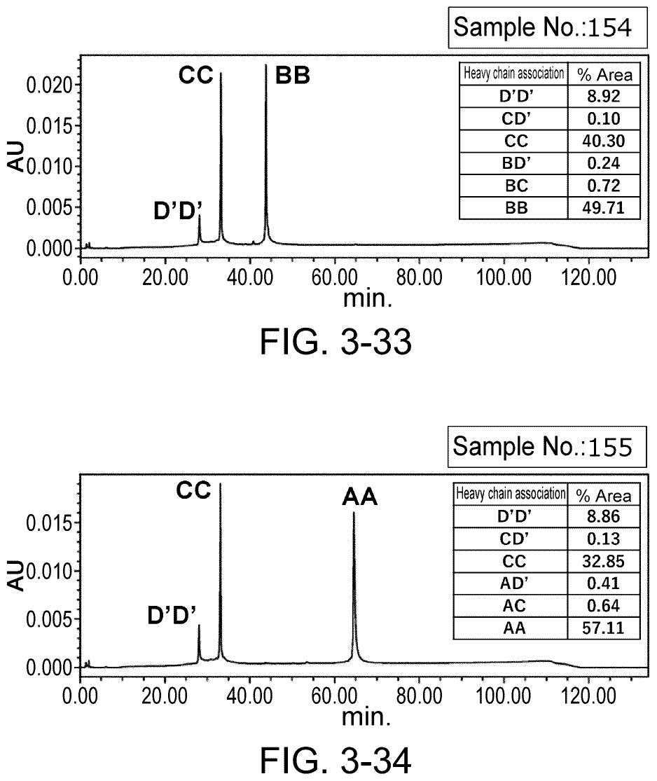

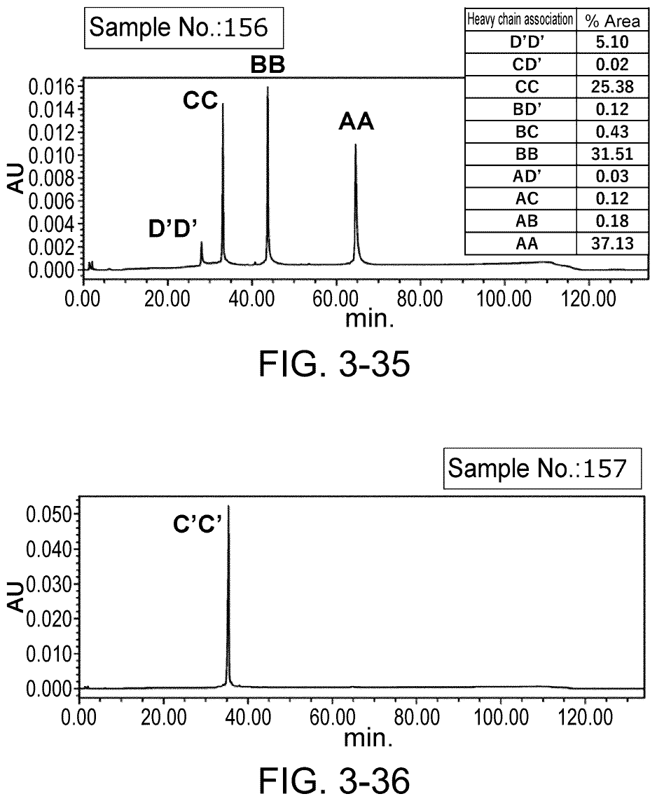

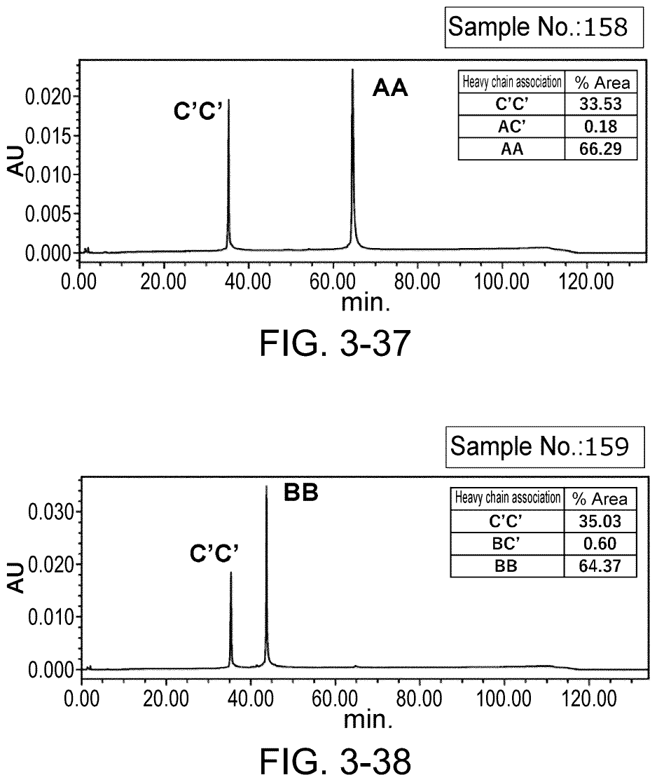

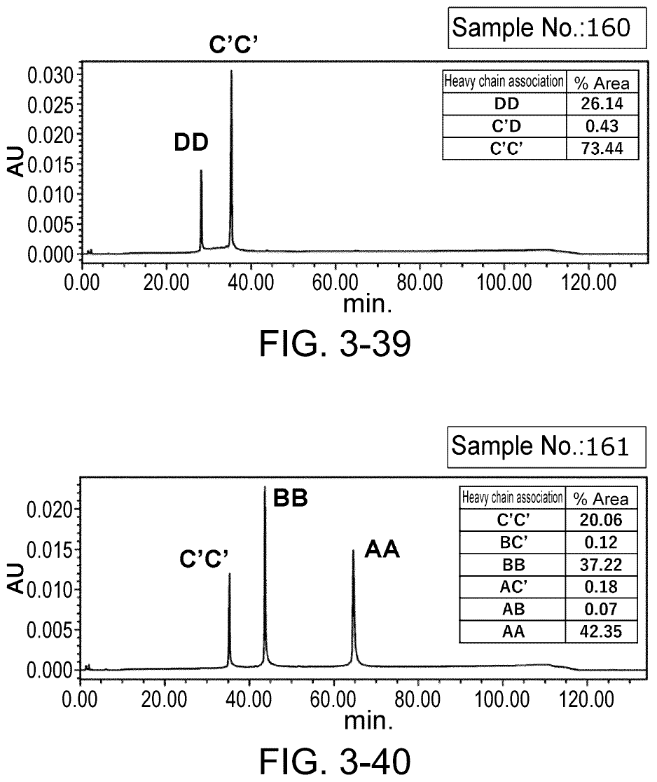

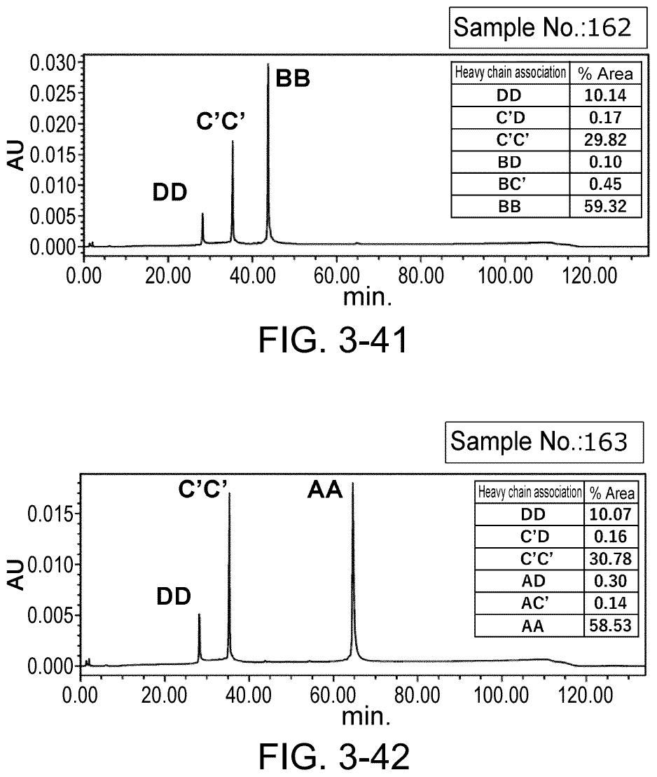

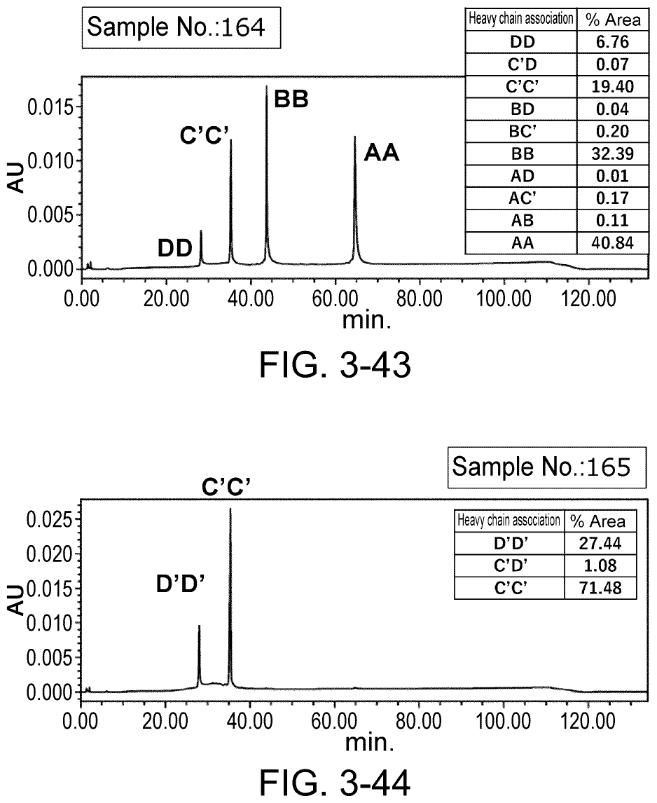

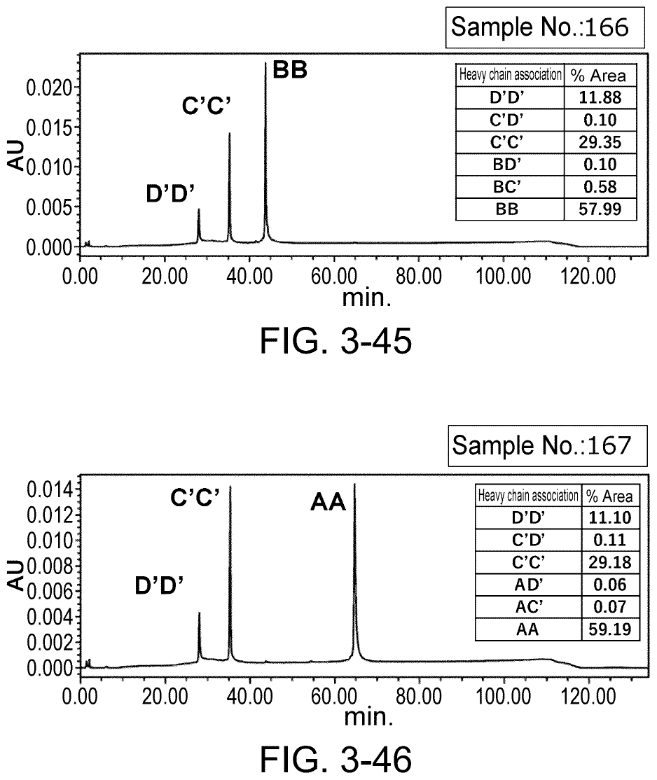

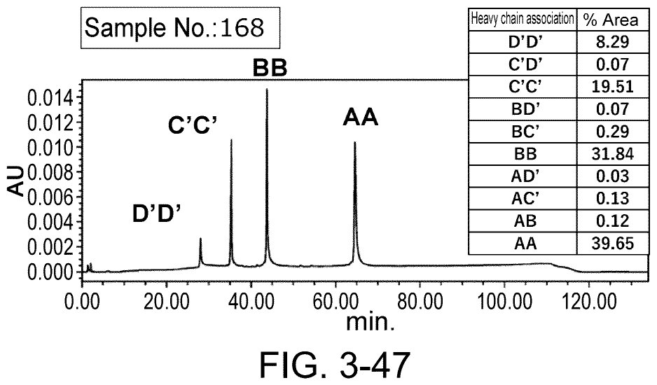

In FIG. 5-3, the elution positions of reference antibodies with CH3 modification, and the ability of heavy chain homodimerization under coexpression of full-length heavy chains and Fc fragments, are analyzed.

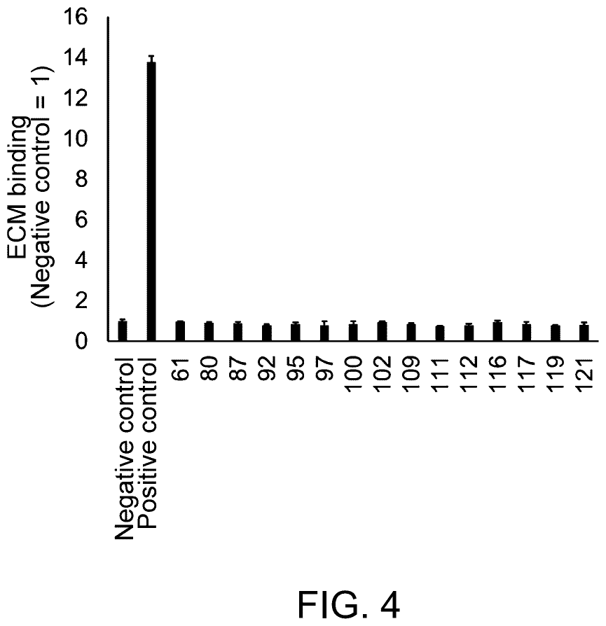

In FIG. 5-4, the elution positions of reference antibodies with CH3 modification, and the ability of heavy chain homodimerization under coexpression of full-length heavy chains and Fc fragments, are analyzed.

In FIG. 5-5, the elution positions of reference antibodies without CH3 modification, and the ability of heavy chain homodimerization under coexpression of full-length heavy chains and Fc fragments, are analyzed.

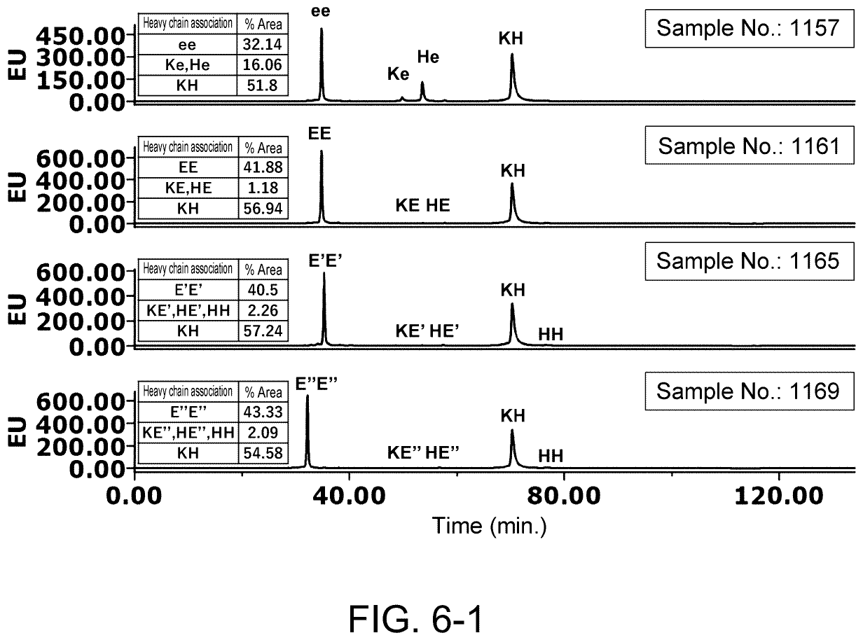

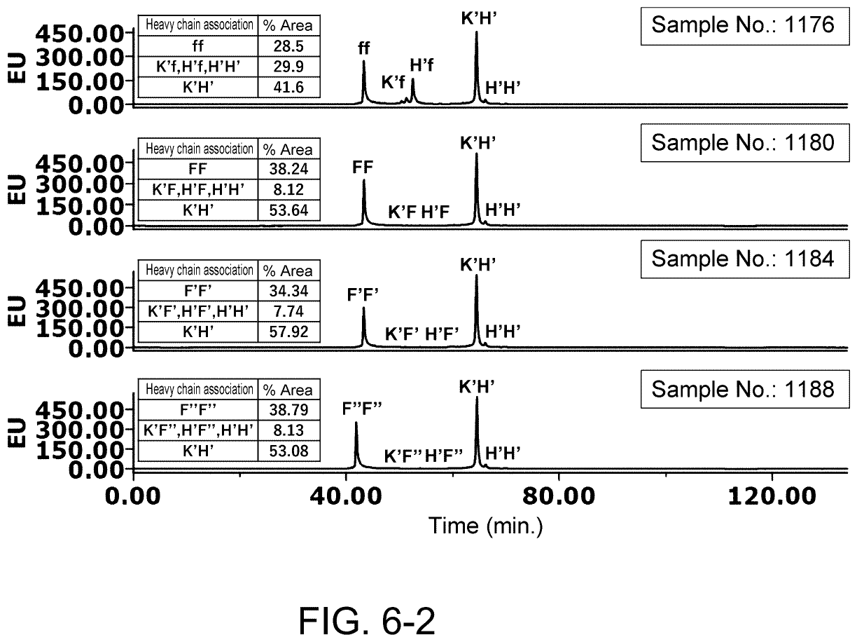

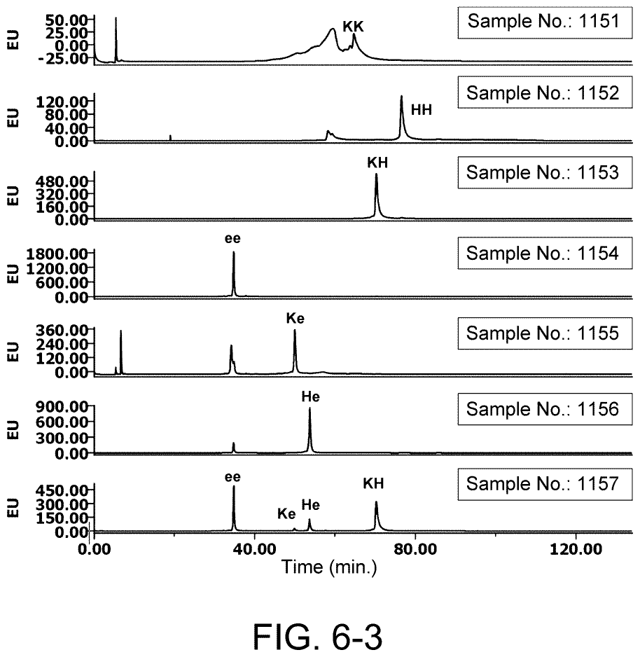

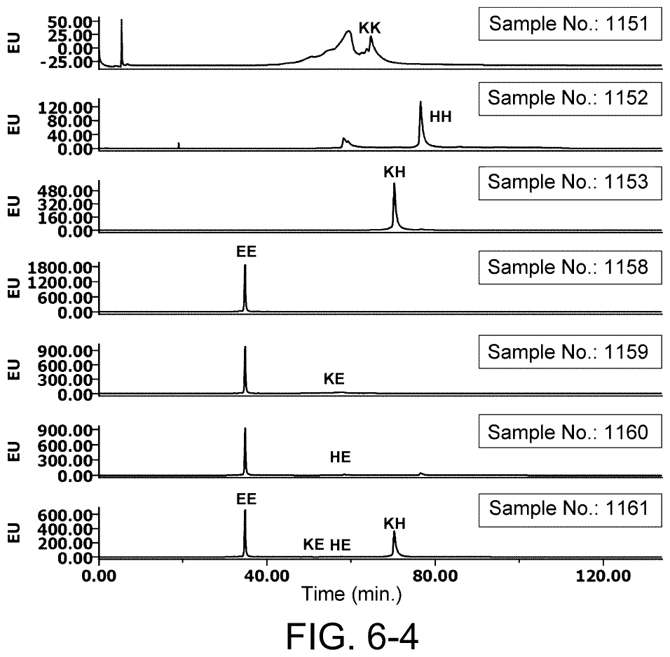

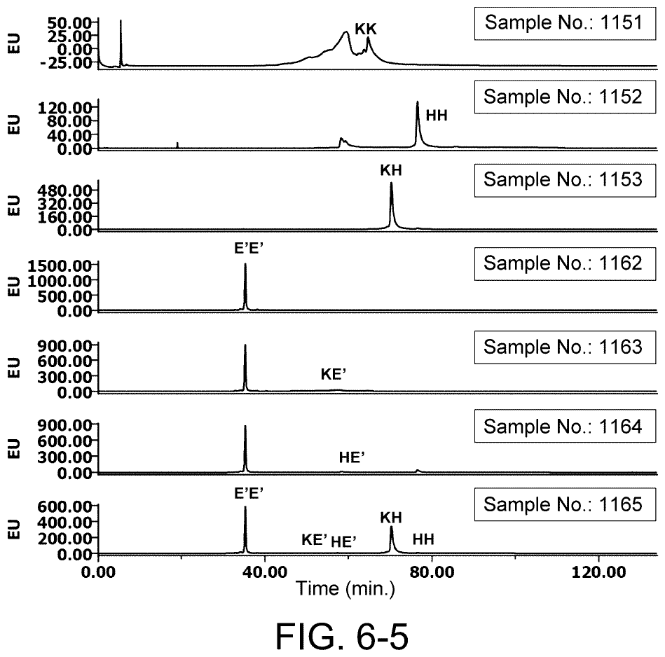

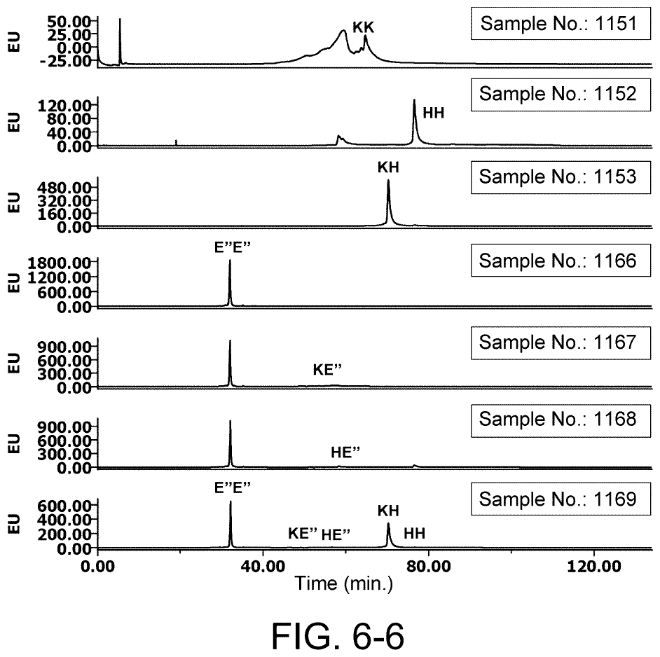

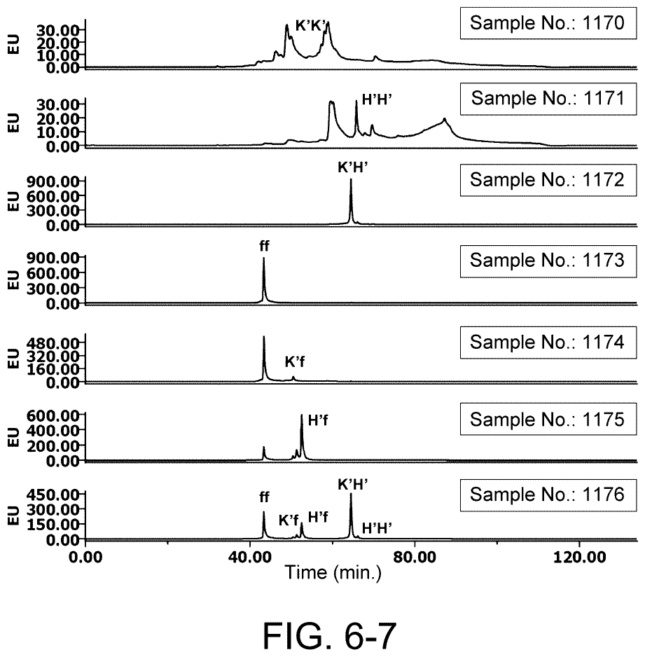

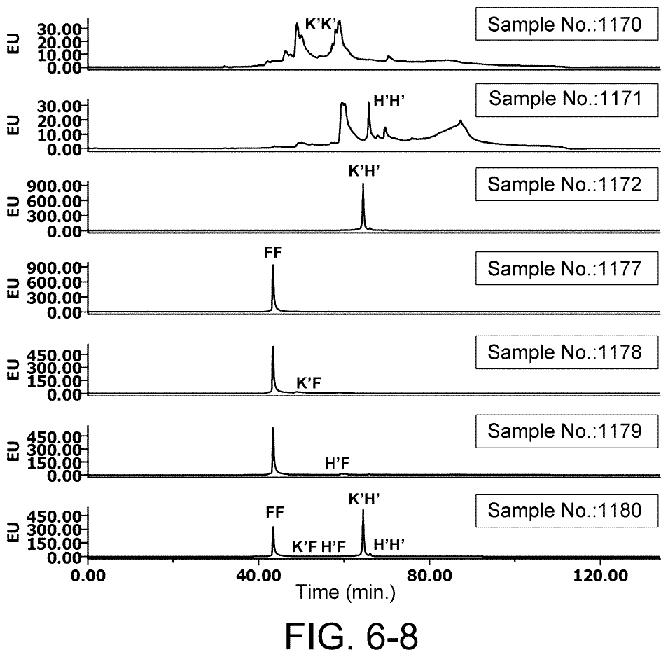

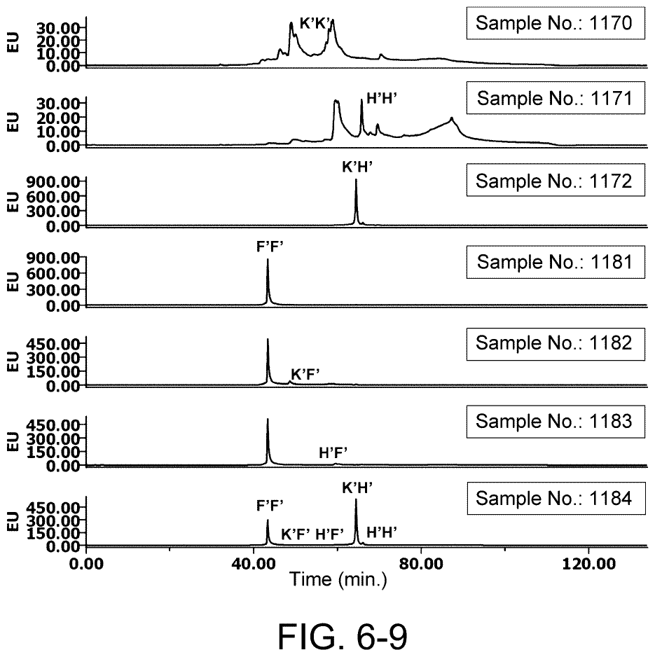

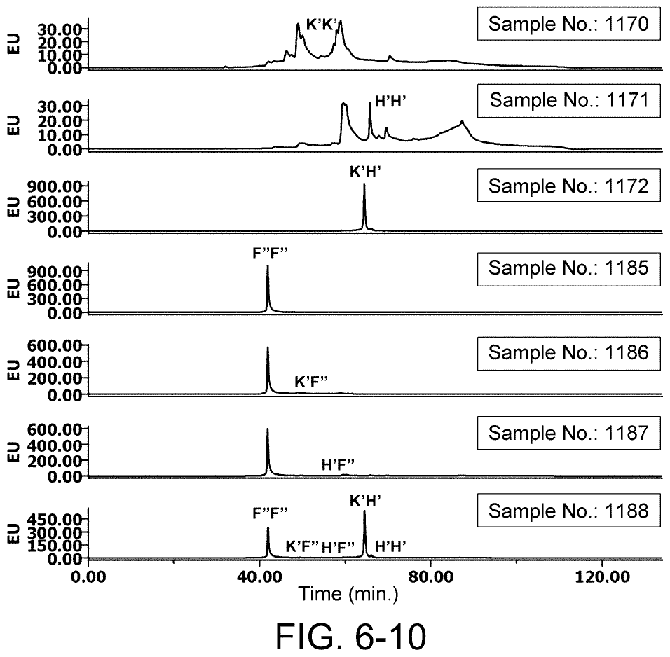

In FIG. 5-6, the elution positions of reference antibodies with CH3 modification, and the ability of heavy chain homodimerization under coexpression of full-length heavy chains and Fc fragments, are analyzed.

In FIG. 5-7, the elution positions of reference antibodies with CH3 modification, and the ability of heavy chain homodimerization under coexpression of full-length heavy chains and Fc fragments, are analyzed.

In FIG. 5-8, the elution positions of reference antibodies with CH3 modification, and the ability of heavy chain homodimerization under coexpression of full-length heavy chains and Fc fragments, are analyzed.

In FIG. 5-9, the elution positions of reference antibodies with CH3 modification, and the ability of heavy chain homodimerization under coexpression of full-length heavy chains and Fc fragments, are analyzed.

In FIG. 5-10, the elution positions of reference antibodies without CH3 modification, and the ability of heavy chain homodimerization under coexpression of full-length heavy chains and Fc fragments, are analyzed.

In FIG. 5-11, the elution positions of reference antibodies with CH3 modification, and the ability of heavy chain homodimerization under coexpression of full-length heavy chains and Fc fragments, are analyzed.

In FIG. 5-12, the elution positions of reference antibodies with CH3 modification, and the ability of heavy chain homodimerization under coexpression of full-length heavy chains and Fc fragments, are analyzed.

In FIG. 5-13, the elution positions of reference antibodies with CH3 modification, and the ability of heavy chain homodimerization under coexpression of full-length heavy chains and Fc fragments, are analyzed.

In FIG. 5-14, the elution positions of reference antibodies with CH3 modification, and the ability of heavy chain homodimerization under coexpression of full-length heavy chains and Fc fragments, are analyzed.

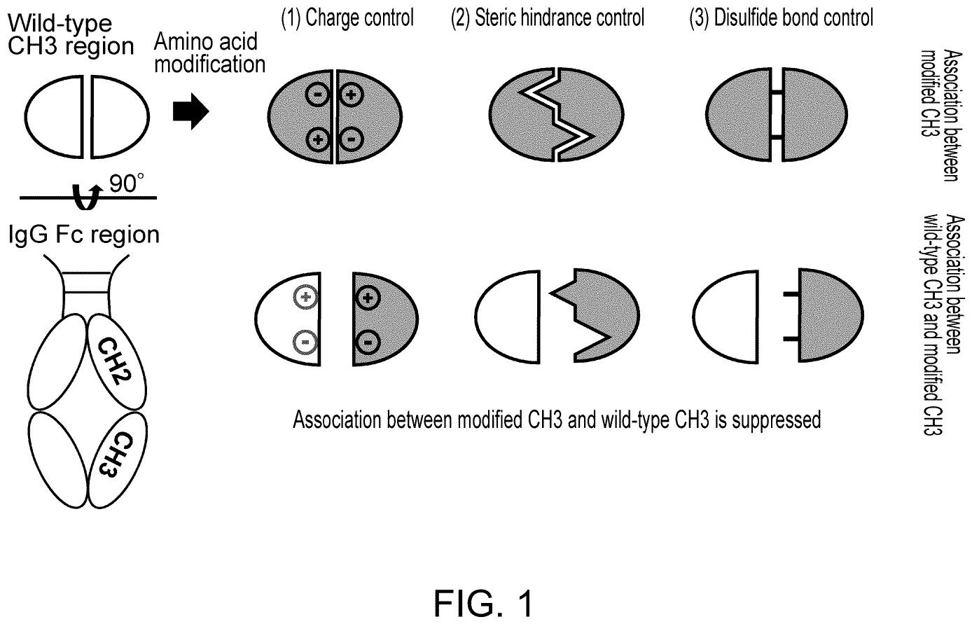

FIG. 6 (FIG. 6-1 to FIG. 6-10) shows the evaluation of the independent heavy chain heteromeric association ability and heavy chain homomeric association ability of antibodies by CIEX analysis. FIG. 6-1 shows the result of analysis when coexpressing one homodimeric antibody and one heterodimeric antibody for which heteromeric association was promoted by knobs-into-holes modifications. In each of the cases where the homodimer had no CH3 modification (Sample No. 1157) and had CH3 modifications (Samples Nos. 1161, 1165, and 1169), the area ratios of homodimer, heterodimer, and unintended heavy chain multimer were calculated. In all cases, the pair containing modifications for promoting homodimerization suppressed unintended heavy chain association as compared to the case of no CH3 modification.

FIG. 6-2 shows the result of analysis when coexpressing one homodimeric antibody and one heterodimeric antibody for which heteromeric association was promoted by knobs-into-holes modifications including disulfide bonds. In each of the cases where the homodimer had no CH3 modification (Sample No. 1176) and had CH3 modifications (Samples Nos. 1180, 1184, and 1188), the area ratios of homodimer, heterodimer, and unintended heavy chain multimer were calculated. In all cases, the pair containing modifications for promoting homodimerization suppressed unintended heavy chain association as compared to the case of no CH3 modification.

FIG. 6-3 shows the result of peak assignment in the CIEX analysis of Samples No. 1157. The plasmids used for expression of Sample No. 1157 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

FIG. 6-4 shows the result of peak assignment in the CIEX analysis of Samples No. 1161. The plasmids used for expression of Sample No. 1161 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

FIG. 6-5 shows the result of peak assignment in the CIEX analysis of Samples No. 1165. The plasmids used for expression of Sample No. 1165 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

FIG. 6-6 shows the result of peak assignment in the CIEX analysis of Samples No. 1169. The plasmids used for expression of Sample No. 1169 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

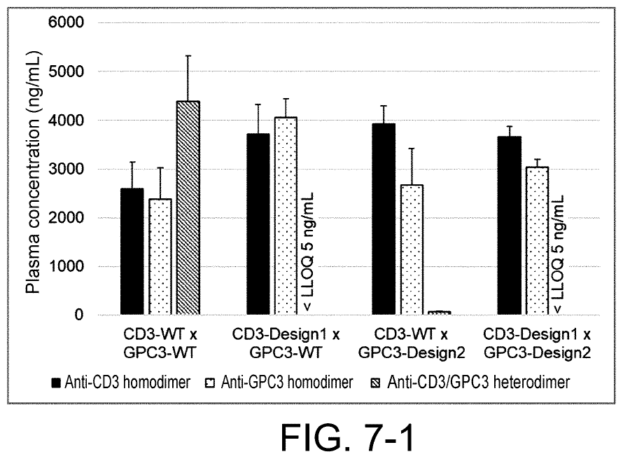

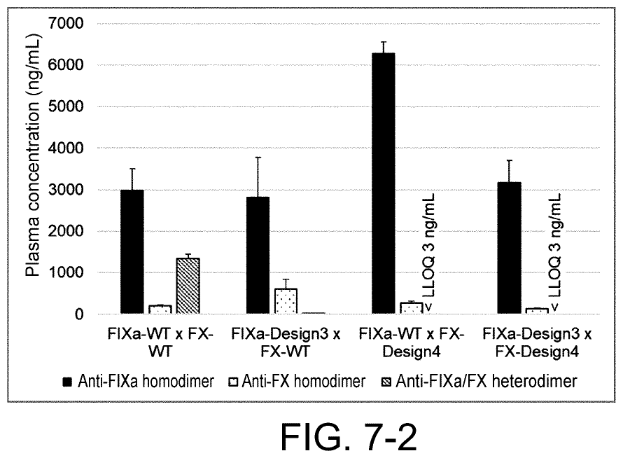

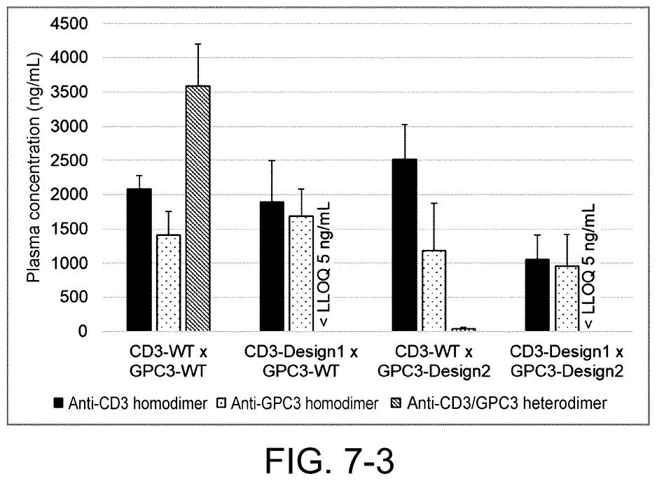

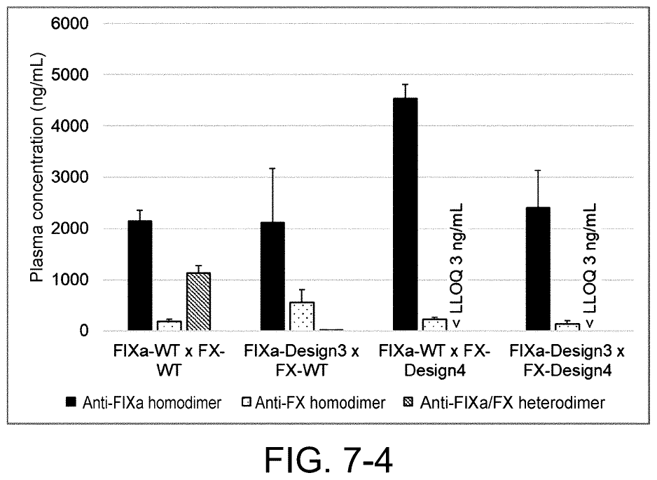

FIG. 6-7 shows the result of peak assignment in the CIEX analysis of Samples No. 1176. The plasmids used for expression of Sample No. 1176 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

FIG. 6-8 shows the result of peak assignment in the CIEX analysis of Samples No. 1180. The plasmids used for expression of Sample No. 1180 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

FIG. 6-9 shows the result of peak assignment in the CIEX analysis of Samples No. 1184. The plasmids used for expression of Sample No. 1184 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

FIG. 6-10 shows the result of peak assignment in the CIEX analysis of Samples No. 1188. The plasmids used for expression of Sample No. 1188 were expressed in various combinations and each reference antibody thus produced was analyzed. The peaks for the heavy chain homodimer and heavy chain heterodimer of interest and other unintended peaks were assigned. The assigned heavy chain association is indicated at each peak in the figure.

FIG. 7-1 shows the plasma concentration of the anti-CD3 homodimer, anti-GPC3 homodimer, and anti-CD3/GPC3 heterodimer on day 3 after administration.

FIG. 7-2 shows the plasma concentration of the anti-FIXa homodimer, anti-FX homodimer, and anti-FIXa/FX heterodimer on day 3 after administration.

FIG. 7-3 shows the plasma concentration of the anti-CD3 homodimer, anti-GPC3 homodimer, and anti-CD3/GPC3 heterodimer on day 7 after administration.

FIG. 7-4 shows the plasma concentration of the anti-FIXa homodimer, anti-FX homodimer, and anti-FIXa/FX heterodimer on day 7 after administration.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

The definitions and detailed description below are provided to facilitate understanding of the present disclosure illustrated herein.

### Definitions

### Amino Acids

Herein, amino acids are described by one- or three-letter codes or both, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V.

### Modification of Amino Acids

For amino acid modification (herein also referred to as “amino acid substitution” or “amino acid mutation”) in the amino acid sequence of a polypeptide, known methods such as site-directed mutagenesis methods (Kunkel et al. (Proc. Natl. Acad. Sci. USA (1985) 82, 488-492)) and overlap extension PCR may be appropriately employed. Furthermore, several known methods may also be employed as amino acid alteration methods for substitution to non-natural amino acids (Annu Rev. Biophys. Biomol. Struct. (2006) 35, 225-249; and Proc. Natl. Acad. Sci. U.S.A. (2003) 100 (11), 6353-6357). For example, it is possible to use a cell-free translation system (Clover Direct (Protein Express)) containing a tRNA which has a non-natural amino acid bound to a complementary amber suppressor tRNA of one of the stop codons, the UAG codon (amber codon).

In the present specification, the meaning of the term “and/or” when describing the site of amino acid modification includes every combination where “and” and “or” are suitably combined. Specifically, for example, “the amino acids at positions x, y, and/or z are substituted” includes the following variation of amino acid alterations: amino acid(s) at (a) position x, (b) position y, (c) position z, (d) positions x and y, (e) positions x and z, (f) positions y and z, and (g) positions x, y, and z.

Furthermore, herein, as an expression showing modification of amino acids, an expression that shows before and after a number indicating a specific position, one-letter or three-letter codes for amino acids before and after modification, respectively, may be used appropriately. For example, the modification E345K used when substituting an amino acid contained in an antibody variable region indicates substitution of Glu (E) at position 345 (according to EU numbering) with Lys (K). That is, the number shows the amino acid position according to EU numbering, the one-letter or three-letter amino-acid code written before the number shows the amino acid before substitution, and the one-letter or three-letter amino-acid code written after the number shows the amino acid after substitution.

### Polypeptides

As used herein, term “polypeptide” refers to a molecule composed of monomers (amino acids) linearly linked by amide bonds (also known as peptide bonds). The term “polypeptide” does not refer to a product with a specific length. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be, in one embodiment, of a size of 10 or more, 20 or more, 25 or more, 50 or more, 75 or more, 100 or more, 200 or more, 500 or more, or 1,000 or more amino acids.

A polypeptide of the present disclosure is, in one embodiment, produced in a living organism. For example, a polypeptide of the present disclosure can be produced in a living organism by introducing a nucleic acid encoding the polypeptide of the present disclosure into the living organism. In one embodiment, a polypeptide of the present disclosure is a polypeptide expressed in a living organism.

### Recombinant Methods and Compositions

Polypeptides which are antibodies or antigen-binding molecules may be produced using recombinant methods and compositions, e.g., as described in U.S. Pat. No. 4,816,567. In one embodiment, isolated nucleic acid encoding a polypeptide as described herein is provided. In one embodiment, such nucleic acid may encode a polypeptide having a heavy chain Fc region. In a further embodiment, one or more vectors (e.g., expression vectors) comprising such nucleic acid are provided. In a further embodiment, a host cell comprising such nucleic acid is provided. In one of such embodiments, the host cell comprises (e.g., has been transformed with) a vector comprising a nucleic acid encoding an amino acid sequence comprising a heavy chain Fc region. In one embodiment, the host cell is eukaryotic, e.g. a Chinese Hamster Ovary (CHO) cell or lymphoid cell (e.g., Y0, NS0, Sp2/0 cell). In one embodiment, a method for producing the polypeptide for which association is controlled according to the present disclosure is provided, wherein the method comprises culturing a host cell comprising a nucleic acid encoding the polypeptide, as provided above, under conditions suitable for expression of the polypeptide, and optionally recovering the polypeptide from the host cell (or host cell culture medium).

For recombinant production of a polypeptide described herein (in one embodiment, an antibody), a nucleic acid encoding the polypeptide is isolated and inserted into one or more vectors for further cloning and/or expression in a host cell. Such nucleic acids may be readily isolated and sequenced using conventional procedures, e.g., by using oligonucleotide probes that are capable of binding specifically to a gene encoding the heavy chain Fc region.

Suitable host cells for cloning or expression of vectors encoding polypeptides (in one embodiment, antibodies) include prokaryotic or eukaryotic cells described herein. For example, polypeptides (in one embodiment, antibodies) may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of polypeptide (antibody) fragments in E. coli.) After expression, the polypeptide may be isolated from the bacterial cell paste in a soluble fraction and can be further purified.

In addition to prokaryotes, eukaryotic microbes such as filamentous fungi or yeast are suitable cloning or expression hosts for polypeptide (antibody)-encoding vectors, including fungi and yeast strains whose glycosylation pathways have been “humanized,” resulting in the production of a polypeptide (an antibody in one embodiment) with a partially or fully human glycosylation pattern. (See Gerngross, Nat. Biotech. 22:1409-1414 (2004), and Li et al., Nat. Biotech. 24:210-215 (2006).)

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. Reference can be made to, e.g., U.S. Pat. Nos. 5,959,177, 6,040,498, 6,420,548, 7,125,978, and 6,417,429 (describing PLANTIBODIES™ technology for producing polypeptides (antibodies in one embodiment) in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR″ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for polypeptide (antibody) production, reference can be made to, e.g., Yazaki and Wu, Methods in Molecular Biology, Vol. 248 (B.K.C. Lo, ed., Humana Press, Totowa, NJ), pp. 255-268 (2003).

Recombinant production of a polypeptide (in one embodiment, an antibody) described herein could be done with methods similar to those described above, by using a host cell that comprises (e.g., has been transformed with) one or more vectors comprising a nucleic acid that encodes an amino acid sequence comprising the whole polypeptide (antibody) or part of the polypeptide (antibody).

The polypeptides of the present disclosure (in one embodiment, antibodies) include polypeptides that have undergone posttranslational modification. Examples of the polypeptides thereof of the present disclosure undergoing posttranslational modification include, in the case of the polypeptides being antibodies, antibodies which have undergone pyroglutamylation at the N terminal of the heavy chain variable region and/or deletion of lysine at the C terminal of the heavy chain. It is known in the field that such posttranslational modification due to pyroglutamylation at the N terminal and deletion of lysine at the C terminal does not have any influence on the activity of the antibody (Analytical Biochemistry, 2006, Vol. 348, p. 24-39).

As used herein, the terms “first”, “second”, and such with respect to polypeptides etc., are used for convenience of distinguishing. Use of these terms is not intended to confer a specific order or orientation unless explicitly so stated.

A polypeptide may be a TCR (T cell receptor) or part of a TCR.

### Expression in Cells in Living Organisms

In one embodiment, a polypeptide of the present disclosure may be expressed in cells in living organisms.

In one embodiment, expression of a polypeptide of the present disclosure in cells is achieved by introducing a nucleic acid encoding the polypeptide of the present disclosure into target cells. Any standard method of introducing nucleic acids into a cell may be used. Such methods include, for example, microinjection, ballistic injection, electroporation, calcium phosphate precipitation, liposomes, and transfection with retroviral, adenoviral, adeno-associated viral and vaccinia vectors carrying the nucleic acid of interest.

In one embodiment, a nucleic acid encoding a polypeptide of the present disclosure may be introduced into cells of a subject (in one embodiment, a human) by in vivo and ex vivo methods. In one example of in vivo delivery, a nucleic acid is injected directly into the subject e.g., at the site where treatment is required. In a further example of in vivo delivery, a nucleic acid is introduced into a cell using transfection with a viral vector (such as adenovirus, Herpes simplex I virus, or adeno-associated virus) and a lipid-based system (useful lipids for lipid-mediated transfer of the gene are DOTMA, DOPE and DC-Chol, for example) (For review of certain gene marking and gene therapy protocols, see Anderson et al., Science 256:808-813 (1992), and WO 93/25673 and the references cited therein). In an example of ex vivo treatment, a subject's cells are removed, a nucleic acid is introduced into those isolated cells, and the modified cells are administered to the subject either directly or, for example, encapsulated within porous membranes which are implanted into the subject (see, e.g., U.S. Pat. Nos. 4,892,538 and 5,283,187). A commonly used vector for ex vivo delivery of a nucleic acid is, in one embodiment, a retroviral vector.

### Intrabody

In one embodiment, a polypeptide of the present disclosure can be expressed intracellularly as an intrabody (intracellularly expressed antibody).

The term “intrabody,” as used herein, refers to an antibody or antigen-binding portion thereof that is expressed intracellularly and that is capable of selectively binding to a target molecule, as described, e.g., in Marasco, Gene Therapy 4:11-15 (1997); Kontermann, Methods 34:163-170 (2004); U.S. Pat. Nos. 6,004,940 and 6,329,173; U.S. Patent Application Publication No. 2003/0104402, and PCT Publication No. WO2003/077945 (see also, for example, WO96/007321 published Mar. 14, 1996, concerning the use of gene therapy to generate intracellular antibodies).

In one embodiment, intracellular expression of an intrabody may be effected by introducing a nucleic acid encoding the antibody or antigen-binding portion thereof into a target cell. One or more nucleic acids encoding all or a portion of an antibody including a polypeptide of the present disclosure can be delivered to a target cell, such that one or more intrabodies are expressed.

In one embodiment, a polypeptide of the present disclosure for which association between polypeptides is controlled can be used for intracellular production of intrabodies.

### Single-Domain Antibody

In one embodiment, a polypeptide of the present disclosure may be a polypeptide having a single-domain antibody.

As used herein, the term “single-domain antibody” is not limited by its structure as long as the domain can exert antigen binding activity by itself. It is known that a general antibody, for example, an IgG antibody, exhibits antigen binding activity in a state where a variable region is formed by the pairing of VH and VL, whereas the own domain structure of the single-domain antibody can exert antigen binding activity by itself without pairing with another domain. Usually, the single-domain antibody has a relatively low molecular weight and exists in the form of a monomer.

Examples of the single-domain antibody include, but are not limited to, antigen-binding molecules congenitally lacking a light chain, such as VHH of an animal of the family Camelidae and shark VNAR, and antibody fragments containing the whole or a portion of an antibody VH domain or the whole or a portion of an antibody VL domain. Examples of the single-domain antibody which is an antibody fragment containing the whole or a portion of an antibody VH or VL domain include, but are not limited to, artificially prepared single-domain antibodies originating from human antibody VH or human antibody VL as described in U.S. Pat. No. 6,248,516 B1, etc. In some embodiments of the present disclosure, one single-domain antibody has three CDRs (CDR1, CDR2 and CDR3).

In one embodiment, single-domain antibodies of the present disclosure include scFv-Fc or VHH-Fc.

### Variable Region

In one embodiment, a polypeptide of the present disclosure may be a polypeptide having a variable region.

The term “variable region” or “variable domain” refers to the domain of an antibody heavy or light chain that is involved in binding the antibody to antigen. The variable domains of the heavy chain and light chain (VH and VL, respectively) of a naturally-occurring antibody generally have similar structures, with each domain comprising four conserved framework regions (FRs) and three hypervariable regions (HVRs). (See, e.g., Kindt et al. Kuby Immunology, 6th ed., W. H. Freeman and Co., page 91 (2007).) A single VH or VL domain is capable of conferring antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

### HVR or CDR

In one embodiment, a polypeptide of the present disclosure is an antibody, and may have HVRs or CDRs.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Hypervariable regions (HVRs) are also referred to as “complementarity determining regions” (CDRs), and these terms are used herein interchangeably in reference to portions of the variable region that form the antigen binding regions. Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

HVR-H1, HVR-H2, HVR-H3, HVR-L1, HVR-L2, and HVR-L3 are also mentioned as “H-CDR1”, “H-CDR2”, “H-CDR3”, “L-CDR1”, “L-CDR2”, and “L-CDR3”, respectively.

### Framework

In one embodiment, a polypeptide of the present disclosure is an antibody, and may have a variable region containing a framework.

In one embodiment, a polypeptide of the present disclosure may have a variable region containing a framework.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

### Antibody

In one embodiment, a polypeptide of the present disclosure is an antibody. The term “antibody” herein is used in the broadest sense and encompasses various antibody structures, including but not limited to monoclonal antibodies, polyclonal antibodies, monospecific antibodies, and antibody fragments so long as they exhibit the desired antigen-binding activity.

### Class of Antibody

In one embodiment, a polypeptide of the present disclosure is an antibody, which is not limited to a particular class of antibody.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

### EU Numbering

Unless otherwise indicated, amino acid residues in the light chain constant region are numbered herein according to Kabat et al., and numbering of amino acid residues in the heavy chain constant region is according to the EU numbering system, also called the EU index, as described in Kabat et al., Sequences of Proteins of Immunological Interest, 5th Ed. Public Health Service, National Institutes of Health, Bethesda, MD, 1991.

### Chimeric Antibody

In one embodiment, a polypeptide of the present disclosure may be a chimeric antibody.

The term “chimeric” antibody refers to an antibody in which a portion of the heavy and/or light chain is derived from a particular source or species, while the remainder of the heavy and/or light chain is derived from a different source or species. Similarly, the term “chimeric antibody variable domain” refers to an antibody variable region in which a portion of the heavy and/or light chain variable region is derived from a particular source or species, while the remainder of the heavy and/or light chain variable region is derived from a different source or species.

### Humanized Antibody

In one embodiment, a polypeptide of the present disclosure may be a humanized antibody.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

### Human Antibody

In one embodiment, a polypeptide of the present disclosure may be a human antibody.

A “human antibody” is one which possesses an amino acid sequence which corresponds to that of an antibody produced by a human or a human cell or derived from a non-human source that utilizes human antibody repertoires or other human antibody-encoding sequences.

### Nucleic Acid (Polynucleotide)

In one embodiment, the present disclosure relates to nucleic acids encoding a polypeptide of the present disclosure.

In one embodiment, a nucleic acid is RNA, DNA, or a vector or plasmid carrying the nucleic acid.

“Nucleic acid” or “polynucleotide” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label.

In one embodiment, RNAs of the present disclosure include, for example, mRNAs, genomic RNAs, and circular RNAs.

A polynucleotide of the present disclosure may comprise a 5′-cap structure. The “5′-cap structure” is, for example, a 5′-modified nucleotide, particularly a guanine nucleotide, positioned at the 5′-terminus of RNA, e.g., mRNA. A 5′-cap structure may be linked via a 5′-5′-triphosphate linkage. Examples of the 5′-cap structure are cap0 (methylation of the first nucleobase, e.g., m7GpppN), cap1 (additional methylation of the ribose in the nucleotide adjacent to m7GpppN), cap2 (additional methylation of the ribose of the second nucleotide downstream of m7GpppN), cap3 (additional methylation of the ribose of the third nucleotide downstream of m7GpppN), cap4 (additional methylation of the ribose of the fourth nucleotide downstream of m7GpppN), ARCA (anti-reverse cap analog), modified ARCA (e.g., phosphorothioate-modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

A polynucleotide of the present disclosure may comprise at least one poly (A) sequence and/or at least one poly (C) sequence and/or at least one histone step-loop sequence/structure.

A polynucleotide of the present disclosure may comprise a 5′-untranslated region (UTR) and/or a 3′-untranslated region (UTR). In one embodiment, an untranslated region may include a structure that promotes translation, such as an IRES (internal ribosome entry site). In one embodiment, a nucleic acid encoding a polypeptide of the present disclosure is RNA (e.g., mRNA). In one embodiment, the RNA comprises one or more of the following elements: a coding region (coding sequence; CDS), 5′UTR, 3′UTR, Cap, and poly-A tail. In one embodiment, the coding region comprises a start codon and a stop codon. In an RNA of the present disclosure, in one embodiment, one or more or all of the uridines may be substituted with pseudouridines (N1-methyl pseudouridines).

In one embodiment, a nucleic acid of the present disclosure is DNA. In one embodiment, the DNA is configured such that the aforementioned RNA of the present disclosure is transcribed. In one embodiment, a DNA of the present disclosure is a vector DNA or a plasmid DNA. In one embodiment, the vector or plasmid comprises one or more of the following elements: an ORF (a region encoding a polypeptide of the present disclosure), a promoter, Kozak, a marker gene (in one embodiment, a drug selection gene, a fluorescent marker gene, a reporter gene, or such).

A polynucleotide of the present disclosure may be formulated with a transport agent. The transport agent is, for example, a lipidoid, liposome, lipoplex, lipid nanoparticle, polymeric compound, peptide, protein, cell, nanoparticle mimic, nanotube, or conjugate. A transport agent may contain a lipid. The lipid may be selected from the group consisting of cholesterol, fecosterol, sitosterol, ergosterol, campesterol, stigmasterol, brassicasterol, tomatidine, ursolic acid, alpha-tocopherol, and mixtures thereof.

### Vector

In one embodiment, the present disclosure relates to a vector carrying a nucleic acid of the present disclosure.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors could be introduced into host cells using virus or electroporation. However, introduction of vectors is not limited to in vitro method. For example, vectors could also be introduced into a subject using in vivo method directly.

### Host Cell

In one embodiment, the present disclosure relates to host cells into which a nucleic acid of the present disclosure has been introduced. In one embodiment, a host cell is a cell expressing a polypeptide of the present disclosure. The cell may be a eukaryotic cell or a prokaryotic cell.

The terms “host cell,” “host cell line,” and “host cell culture” are used interchangeably and refer to, for example, cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations.

Further, in one embodiment, the present disclosure relates to polypeptides expressed from the aforementioned host cell.

### Fc Region

In the present disclosure, the term “Fc region” or “Fc domain” is used interchangeably, and refers to a region comprising a fragment consisting of a hinge or a portion thereof and CH2 and CH3 domains in an antibody molecule. The Fc region of IgG class means, but is not limited to, a region from, for example, cysteine 226 (EU numbering) to the C terminus or proline 230 (EU numbering) to the C terminus. The Fc region can be preferably obtained by the partial digestion of, for example, an IgG1, IgG2, IgG3, or IgG4 monoclonal antibody with a proteolytic enzyme such as pepsin followed by the re-elution of a fraction adsorbed on a protein A column or a protein G column. Such a proteolytic enzyme is not particularly limited as long as the enzyme is capable of digesting a whole antibody to restrictively form Fab or F(ab′) 2 under appropriately set reaction conditions (e.g., pH) of the enzyme. Examples thereof can include pepsin and papain.

In one embodiment, an Fc region derived from, for example, naturally occurring IgG can be used as an “Fc region” prior to modification. In this context, the naturally occurring IgG means a polypeptide that contains an amino acid sequence identical to that of IgG found in nature and belongs to a class of an antibody substantially encoded by an immunoglobulin gamma gene. The naturally occurring human IgG means, for example, naturally occurring human IgG1, naturally occurring human IgG2, naturally occurring human IgG3, or naturally occurring human IgG4. The naturally occurring IgG also includes variants or the like spontaneously derived therefrom. A plurality of allotype sequences based on gene polymorphism are described as the constant regions of human IgG1, human IgG2, human IgG3, and human IgG4 antibodies in Sequences of proteins of immunological interest, NIH Publication No. 91-3242, any of which can be used in the present disclosure. Particularly, the sequence of human IgG1 may have DEL or EEM as an amino acid sequence of EU numbering positions 356 to 358.

In one embodiment described herein, the Fc region is an IgG Fc region. In one embodiment, the Fc region is an Fc region of IgG1, IgG2, IgG3, or IgG4. Furthermore, in one embodiment, the Fc region is a human IgG1 Fc region.

In one embodiment, a polypeptide having an Fc region of the present disclosure shows a binding affinity for human FcRn or FcγR that is comparable to that of a naturally-occurring (wild-type) human IgG1 Fc region.

In one embodiment, a polypeptide having an Fc region of the present disclosure shows a binding affinity for human FcRn or FcγR that is similar to that of the polypeptide prior to modification.

In one embodiment, an Fc region (Fc domain) exhibits a binding affinity for an Fc receptor that is similar to that of a naturally-occurring IgG1 Fc region. In one embodiment, an Fc region (or a polypeptide having the Fc region) exhibits 80% or higher, preferably 90% or higher, more preferably 95% or higher, and most preferably 98% or higher binding affinity for an Fc receptor, as compared to a naturally-occurring IgG1 Fc region (or a polypeptide comprising a naturally-occurring IgG1 Fc region).

In one embodiment, an Fc region (or a polypeptide having the Fc region) binds to an Fc receptor. In one embodiment, the Fc receptor is an Fcγ receptor. In one embodiment, the Fc receptor is a human Fc receptor. In one embodiment, the Fc receptor is an activating Fc receptor. In a specific embodiment, the Fc receptor is an activating human Fcγ receptor, more specifically human FcγRIIIa, FcγRI, or FcγRIIa.

In one embodiment, the Fc region (Fc domain) of a polypeptide of the present disclosure comprises a modification that does not reduce the binding affinity of the Fc region for an Fc receptor. In one embodiment, a modification in a polypeptide of the present disclosure does not reduce the binding affinity of the Fc region for human FcRn. In embodiment, such modifications include those shown in Table 8.

Modified Fc regions (mutant Fc domains) can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors and such may be obtained by recombinant expression.

### Fc Receptor

The term “Fc receptor” or “FcR” refers to a receptor that binds to the Fc region of an antibody. In some embodiments, an FcR is a naturally-occurring human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “Fc receptor” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18 (12): 592-598 (1997); Ghetie et al., Nature Biotechnology, 15 (7): 637-640 (1997); Hinton et al., J. Biol. Chem. 279 (8): 6213-6216 (2004); WO 2004/92219 (Hinton et al.).

### Fcγ Receptor

Fcγ receptor refers to a receptor capable of binding to the Fc domain of monoclonal IgG1, IgG2, IgG3, or IgG4 antibodies, and includes all members belonging to the family of proteins substantially encoded by an Fcγ receptor gene. In human, the family includes FcγRI (CD64) including isoforms FcγRIa, FcγRIb and FcγRIc; FcγRII (CD32) including isoforms FcγRIIa (including allotype H131 and R131), FcγRIIb (including FcγRIIb-1 and FcγRIIb-2), and FcγRIIc; and FcγRIII (CD16) including isoform FcγRIIIa (including allotype V158 and F158) and FcγRIIIb (including allotype FcγRIIIb-NA1 and FcγRIIIb-NA2); as well as all unidentified human Fcγ receptors, Fcγ receptor isoforms, and allotypes thereof. However, Fcγ receptor is not limited to these examples. Without being limited thereto, Fcγ receptor includes those derived from humans, mice, rats, rabbits, and monkeys. Fcγ receptor may be derived from any organisms. Mouse Fcγ receptor includes, without being limited to, FcγRI (CD64), FcγRII (CD32), FcγRIII (CD16), and FcγRIII-2 (CD16-2), as well as all unidentified mouse Fcγ receptors, Fcγ receptor isoforms, and allotypes thereof. Such preferred Fcγ receptors include, for example, human FcγRI (CD64), FcγRIIA (CD32), FcγRIIB (CD32), FcγRIIIA (CD16), and/or FcγRIIIB (CD16). The polynucleotide sequence and amino acid sequence of FcγRI are shown in RefSeq accession number NM_000566.3 and RefSeq accession number NP_000557.1, respectively; the polynucleotide sequence and amino acid sequence of FcγRIIA are shown in RefSeq accession number BC020823.1 and RefSeq accession number AAH20823.1, respectively; the polynucleotide sequence and amino acid sequence of FcγRIIB are shown in RefSeq accession number BC146678.1 and RefSeq accession number AAI46679.1, respectively; the polynucleotide sequence and amino acid sequence of FcγRIIIA are shown in RefSeq accession number BC033678.1 and RefSeq accession number AAH33678.1, respectively; and the polynucleotide sequence and amino acid sequence of FcγRIIIB are shown in RefSeq accession number BC128562.1 and RefSeq accession number AAI28563.1, respectively. Whether an Fcγ receptor has binding activity to the Fc domain of a monoclonal IgG1, IgG2, IgG3, or IgG4 antibody can be assessed by ALPHA screen (Amplified Luminescent Proximity Homogeneous Assay), surface plasmon resonance (SPR)-based BIACORE method, and others (Proc. Natl. Acad. Sci. USA (2006) 103 (11), 4005-4010), in addition to the above-described FACS and ELISA formats.

### Fcγ Receptor-Binding Activity

The binding activity of Fc region (Fc domain) to any of the Fcγ receptors FcγRI, FcγRIIA, FcγRIIB, FcγRIIIA, and/or FcγRIIIB can be assessed by using the above-described FACS and ELISA formats as well as ALPHA screen (Amplified Luminescent Proximity Homogeneous Assay) and surface plasmon resonance (SPR)-based BIACORE method (Proc. Natl. Acad. Sci. USA (2006) 103 (11), 4005-4010).

ALPHA screen is performed by the ALPHA technology based on the principle described below using two types of beads: donor and acceptor beads. A luminescent signal is detected only when molecules linked to the donor beads interact biologically with molecules linked to the acceptor beads and when the two beads are located in close proximity. Excited by laser beam, the photosensitizer in a donor bead converts oxygen around the bead into excited singlet oxygen. When the singlet oxygen diffuses around the donor beads and reaches the acceptor beads located in close proximity, a chemiluminescent reaction within the acceptor beads is induced. This reaction ultimately results in light emission. If molecules linked to the donor beads do not interact with molecules linked to the acceptor beads, the singlet oxygen produced by donor beads do not reach the acceptor beads and chemiluminescent reaction does not occur.

For example, a biotin-labeled antigen-binding molecule or antibody is immobilized to the donor beads and glutathione S-transferase (GST)-tagged Fcγ receptor is immobilized to the acceptor beads. In the absence of a polypeptide comprising a competitive mutant Fc domain, Fcγ receptor interacts with a polypeptide comprising a wild-type Fc region, inducing a signal of 520 to 620 nm as a result. The polypeptide having a non-tagged mutant Fc region competes with the polypeptide comprising a wild-type Fc region for the interaction with Fcγ receptor. The relative binding affinity can be determined by quantifying the reduction of fluorescence as a result of competition. Methods for biotinylating polypeptides such as antibodies using Sulfo-NHS-biotin or the like are known. Appropriate methods for adding the GST tag to an Fcγ receptor include methods that involve fusing polypeptides encoding Fcγ receptor and GST in-frame, expressing the fused gene using cells introduced with a vector carrying the gene, and then purifying using a glutathione column. The induced signal can be preferably analyzed, for example, by fitting to a one-site competition model based on nonlinear regression analysis using software such as GRAPHPAD PRISM (GraphPad; San Diego).

One of the substances for observing their interaction is immobilized as a ligand onto the gold thin layer of a sensor chip. When light is shed on the rear surface of the sensor chip so that total reflection occurs at the interface between the gold thin layer and glass, the intensity of reflected light is partially reduced at a certain site (SPR signal). The other substance for observing their interaction is injected as an analyte onto the surface of the sensor chip. The mass of immobilized ligand molecule increases when the analyte binds to the ligand. This alters the refraction index of solvent on the surface of the sensor chip. The change in refraction index causes a positional shift of SPR signal (conversely, the dissociation shifts the signal back to the original position). In the Biacore system, the amount of shift described above (i.e., the change of mass on the sensor chip surface) is plotted on the vertical axis, and thus the change of mass over time is shown as measured data (sensorgram). Kinetic parameters (association rate constant (ka) and dissociation rate constant (kd)) are determined from the curve of sensorgram, and affinity (KD) is determined from the ratio between these two constants. Inhibition assay is preferably used in the BIACORE methods. Examples of such inhibition assay are described in Proc. Natl. Acad. Sci. USA (2006) 103 (11), 4005-4010.

### Maintenance/Retention of Fc Functions and Physicochemical Properties

In one embodiment, a polypeptide having a modified Fc region of the present disclosure retains a function of Fc.

In one embodiment, a polypeptide having a modified Fc region of the present disclosure maintains a physicochemical property of a polypeptide having a wild-type Fc region (in one embodiment, a wild-type IgG antibody, e.g., wild-type IgG1). In one embodiment, the physicochemical properties include the monomer ratio, thermal stability (e.g., thermal transition midpoint (Tm) value), antibody yield, etc., of the polypeptide. In one embodiment, the monomer ratio, Tm value, and polypeptide (antibody) yield of a polypeptide having a modified Fc region of the present disclosure (in one embodiment, an antibody) are not changed, not decreased, or not greatly decreased, as compared to the polypeptide (antibody) before modification.

In one embodiment, “the monomer ratio of a polypeptide is not greatly decreased” means that the monomer ratio is not decreased by 50% or more, preferably 30% or more, more preferably 10% or more, or yet more preferably 5% or more, as compared to a polypeptide having a wild-type Fc region.

The monomer ratio of a polypeptide is measured by the following method: It is evaluated by size exclusion chromatography (SEC) using ACQUITY UPLC H-Class (Waters), in which 50 mM phosphate buffer containing 300 mM sodium chloride, pH7.0 (Isekyu) is used as a running buffer, and TSKgel SuperSW3000 custom-made column (4.6 mm×15 cm, 4 μm, Gel Lot 89R)(TOSOH) is used as an analysis column. Chromatograms are recorded at a wavelength of UV 215 nm. Samples are diluted at 0.1 mg/mL, and 10 μL is injected. The column temperature is set to 30° C., and measurement is performed at a flow rate of 0.35 mL/min for 10 minutes. Data is analyzed using Empower3 (Waters). The peak area ratios (%) of the monomer, associated multimers, and degradation products of the antibody as estimated from an antibody reference sample (e.g., Tocilizumab) are calculated, and the monomer ratio is determined.

In one embodiment, “the Tm of a polypeptide is not greatly decreased” means that the Tm is not decreased by 30° C. or more, preferably 20° C. or more, more preferably 10° C. or more, even more preferably 5° C. or more, or yet more preferably 2° C. or more, as compared to a polypeptide having a wild-type Fc region.

The Tm of a polypeptide (antibody Fc region) is evaluated by differential scanning fluorimetry (DSF). It has already been reported that Tm determined by this method shows good correlation with Tm determined by differential scanning calorimetry, which is a widely-known method for evaluating the thermal stability of antibodies (Journal of Pharmaceutical Science 2010; 4:1707-1720). A 5000×concentrate of SYPRO orange protein gel stain (Invitrogen) was diluted 50 times with 0.5 M PBS (Sigma), and 18 μL of Fc-containing polypeptide solution diluted to 0.1 mg/mL was mixed with 2 μL of this detection dye. The 20-μL mixture was dispensed into a tube for measurement, and its temperature was raised from 30° C. to 99° C. at a rate of 240° C./hr using Rotor-Gene Q (QIAGEN). Fluorescent changes with increasing temperature were observed at 470 nm (excitation wavelength)/555 nm (fluorescence wavelength). Obtained data was used to determine the temperature at which fluorescent transition was seen using Rotor-Gene Q Series Software (QIAGEN), and the lowest value (Tm1) was determined as the Tm value derived from the Fc region. However, if the Tm value derived from the polypeptide domain fused to the Fc region is comparable to or lower than the Tm value of the Fc, the Tm1 value cannot be said to be the Tm derived from the Fc. In this case, it is necessary to assign the Tm derived from the Fc region (CH3, CH2) separately by preparing and comparing the Fc domain alone, or enzymatically cleaving the polypeptide domain fused to the Fc region before measurement.

In one embodiment, “the yield of a polypeptide (antibody) is not greatly decreased” means that the yield of the polypeptide (antibody) is not decreased by 95% or more, preferably 80% or more, more preferably 50% or more, even more preferably 30% or more, or yet more preferably 10% or more, as compared to a polypeptide having a wild-type Fc region.

The yield of a polypeptide (antibody) can be determined by the following method: One mL of 2E+6 cells/mL of Expi293-F cells (Thermo Fisher Scientific) are transfected with a total of 1 μg of plasmids encoding the full-length heavy and light chains of an antibody at a mass ratio of 1:1 or 1:2, and allowed to express them transiently. For transfection, ExpiFectamine293 transfection kit (Thermo Fisher Scientific) is used. After 4 days of transfection, the culture supernatant was collected and purified using MonoSpin ProA (GL science) or MonoSpin ProG (GL science). The amount of the antibody thus purified (mg) is determined as the yield of the polypeptide (antibody). A polypeptide having a wild-type Fc region and a polypeptide having amino acid modifications in the Fc region can be expressed and purified by the same method and their yields can compared to calculate the expression decrease ratio.

In one embodiment, the expression level of a polypeptide (in one embodiment, an antibody) having a modified Fc region of the present disclosure is comparable to that of a wild-type polypeptide (e.g., IgG1). The ratio of monomer formation is also high, and the Tm of the Fc region is also comparable.

In one embodiment, a polypeptide having a modified Fc region of the present disclosure maintains a Fc function of a polypeptide having a wild-type Fc region (in one embodiment, a wild-type IgG antibody, e.g., wild-type IgG1). In one embodiment, the Fc functions include the ability to bind to FcRn or FcγR. In one embodiment, the FcRn-binding ability and/or FcγR-binding ability of a polypeptide (in one embodiment, an antibody) having a modified Fc region of the present disclosure is not changed, not decreased, or not greatly decreased due to modification in the CH3 interface, as compared to the polypeptide (antibody) prior to modification.

In one embodiment, “the FcRn-binding ability of a polypeptide is not greatly decreased” means that the KD value for FcRn does not become 50 times or more greater, preferably 10 times or more greater, more preferably 2 times or more greater, or even more preferably 1.1 times or more greater than that of a polypeptide having a wild-type Fc region.

The KD value for FcRn can be determined by the following method: Binding to human neonatal Fc receptor (FcRn) is evaluated using Biacore T200 (Cytiva). Evaluation is performed at 25° C. using 50 mM phosphate buffer, 150 mM NaCl, 0.05 w/v %-P20, pH6.0, as a running buffer. rProtein L (BioVision) is immobilized onto Series S CM4 (Cytiva) as a ligand-capturing molecule. However, in the case of an antibody molecule that does not bind to Protein L, other capturing molecules such as Protein A and Protein G may be used. An antibody solution prepared with the running buffer is allowed to interact with this CM4 sensor chip to capture about 400 RU of the antibody. The human FcRn protein used in this measurement is prepared by the method described in WO2010107110. Human FcRn is diluted to 0, 250, 500, 1000, 2000, and 4000 nM with the running buffer and allowed to bind to the captured antibody. The chip is regenerated using 10 mM Glycine-HCl (pH 1.5) and repeatedly used to capture antibodies for measurement. The KD (M) of each antibody for FcRn is calculated using Biacore T200 Evaluation Software 3.2.1 with a steady-state model. By calculating the ratio of the KD value of a polypeptide having amino acid modification in the Fc region to that of a polypeptide having a wild-type Fc region, the binding ability to human FcRn can be compared.

In one embodiment, “the FcγR-binding ability of a polypeptide is not greatly decreased” means that the level of binding to FcγR per amount of captured antibody does not become 1/10 or lower, preferably ⅕ or lower, more preferably ½ or lower, or even more preferably ⅔ or lower due to modification in the CH3 interface as compared to a polypeptide having a wild-type Fc region.

The level of binding to FcγRs per amount of captured antibody can be measured by the following method: The binding activity of the produced Fc-modified antibodies to each human Fcγ receptor is evaluated using Biacore T200 (Cytiva). Evaluation was performed at 25° C. using 50 mM phosphate buffer, 150 mM NaCl, 0.05 w/v %-P20, pH7.4, as a running buffer. rProtein L (BioVision) was immobilized onto Series S CM4 (Cytiva) as a ligand-capturing molecule. An antibody solution prepared with the running buffer was allowed to interact with this CM4 sensor chip to capture about 500 RU of the antibody in the case of measurement for human FcγRIa, and 2000 RU in the case of measurement for the other human FcγRs. The human FcγR proteins used in this measurement are prepared by the method described in WO2022220275. Human FcγR was diluted with the running buffer to 8 nM in the case of FcγRIa or 1000 nM in the case of the other FcγRs, and allowed to bind to the captured antibody. The chip is regenerated using 10 mM Glycine-HCl (pH 1.5) and repeatedly used to capture antibodies for measurement. The binding activity of each antibody to each FcγR was evaluated by calculating the level of FcγR-binding (RU) per unit amount of antibody using Biacore T200 Evaluation Software version 3.2.1. By calculating the ratio of the level of FcγR binding (RU) per unit amount of antibody of a polypeptide having amino acid modification in the Fc region to that of a polypeptide having a wild-type Fc region, the ability of binding to the human FcγR can be compared.

In one embodiment, a polypeptide (in one embodiment, an antibody) having a modified Fc region of the present disclosure has both an FcRn-binding ability and an FcγR-binding ability comparable to those of a wild-type polypeptide (e.g., IgG1).

### Affinity

In one embodiment, polypeptides of the present disclosure have affinity between the polypeptides (e.g., between CH3).

In one embodiment, a polypeptide of the present disclosure binds to an FcRn or an FcγR. This binding can be evaluated from the affinity of the polypeptide of the present disclosure for FcRn or FcγR.

“Affinity” refers to the strength of noncovalent interactions between a single binding site of a molecule (e.g., a polypeptide or antibody) and its binding partner (e.g., FcRn). The affinity of a molecule X for its partner Y can generally be represented by the dissociation constant (KD), which is the ratio of dissociation and association rate constants (koff and kon, respectively). Affinity can be measured by well-established methods known in the art, including those described herein. A particular method for measuring affinity is Surface Plasmon Resonance (SPR).

In one embodiment, KD is measured by a radiolabeled antigen binding assay (RIA). In one embodiment, an RIA is performed with the Fab version of an antibody of interest and its antigen. For example, solution binding affinity of a polypeptide for an assay target is measured by equilibrating the polypeptide with a minimal concentration of (¹²⁵I)-labeled antigen in the presence of a titration series of unlabeled antigen, then capturing the bound assay target with an anti-polypeptide antibody-coated plate (see, e.g., Chen et al., J. Mol. Biol. 293:865-881 (1999)).

According to another embodiment, Kd is measured using a BIACORE® surface plasmon resonance assay, such as an assay using BIACORE®-2000 or BIACORE®-3000 (BIAcore, Inc., Piscataway, NJ).

### Control of Association

In one embodiment, the control of association of the present disclosure is control of association between polypeptides of the same type. In one embodiment, it is control of association between heavy chains of the same type or between CH3 domains of the same type.

In one embodiment, the association between a polypeptide having a modified Fc region and a polypeptide having a wild-type Fc region is controlled.

In one embodiment, the association between modified CH3 and wild-type CH3 is controlled.

In one embodiment, the control of association of the present disclosure includes control of association between any polypeptides, for example, any polypeptides comprising an Fc region.

The “control” of association, in one embodiment, refers to the promotion of association between polypeptides into which a modification of the present disclosure has been introduced, or the promotion or elevation of formation of a homodimer of a polypeptide of the present disclosure. (The formation of a homodimer mentioned here refers to formation of a dimer molecule by association of two identical polypeptides. In the case of antibody, it particularly refers to a molecule comprising a component in which any two identical polypeptides comprising an Fc region are associated.) In one embodiment, the “control” of association is suppression or inhibition of formation of a heterodimer of a polypeptide of the present disclosure. The formation of a heterodimer mentioned here refers to a dimer molecule formed by association of two different polypeptides. In the case of antibody, it particularly refers to a molecule comprising a component in which any two different polypeptides comprising an Fc region are associated. Alternatively, it may be referred to as a heteromultimer as a component in which a plurality of different polypeptides are associated.

Polypeptides into which Modifications have been Introduced

In one embodiment, the present disclosure relates to a polypeptide comprising an Fc region into which a modification has been introduced (also referred to as “a polypeptide of the present disclosure”).

In one embodiment, a polypeptide of the present disclosure is an antibody, and may have a variable region or a variable domain.

In one embodiment, the present disclosure relates to a polypeptide having a heavy chain Fc region or heavy chain CH3 into which a modification has been introduced.

In one embodiment, a polypeptide of the present disclosure has a heavy chain Fc region. In one embodiment, a polypeptide comprises, e.g., a CH3 region.

In one embodiment, a polypeptide of the present disclosure is an antibody heavy chain. A polypeptide of the present disclosure may have an antigen-binding domain, a heavy chain variable region, and/or a light chain variable region.

In one embodiment, a polypeptide comprising an Fc region into which a modification has been introduced according to the present disclosure associates more readily with a polypeptide having the modification than with a polypeptide not having the modification.

In one embodiment, a polypeptide comprising an Fc region into which a modification has been introduced according to the present disclosure associates less readily with a polypeptide not having the modification than with a polypeptide having the modification.

In one embodiment, a polypeptide comprising an Fc region into which a modification has been introduced according to the present disclosure less readily forms a heterodimer with another polypeptide not having the modification. In one embodiment, a polypeptide of the present disclosure less readily forms a heterodimer than a homodimer. In one embodiment, a polypeptide of the present disclosure more readily forms a homodimer than a heterodimer.

In one embodiment, a polypeptide comprising an Fc region into which a modification has been introduced according to the present disclosure has an increased ability to form an associated multimer with a polypeptide having the modification as compared to a polypeptide not having the modification.

In one embodiment, a polypeptide comprising an Fc region into which a modification has been introduced according to the present disclosure associates with a polypeptide having the modification.

In one embodiment, a polypeptide of the present disclosure forms a homomer (homodimer, homodimerized molecule).

In one embodiment, a polypeptide of the present disclosure shows a stronger homodimerization-promoting ability than a control (e.g., the polypeptide before modification, wild-type IgG, or such).

In one embodiment, a polypeptide of the present disclosure is a polypeptide for which association between polypeptides is controlled. In one embodiment, the association is association between Fc regions or between CH3 domains.

In one embodiment, a polypeptide of the present disclosure does not associate, or associates less readily, with endogenous IgG or antibody fragments containing an Fc region thereof.

### Modifications to be Introduced

In one embodiment, because of one of the following actions due to an introduced modification, a polypeptide of the present disclosure associates more readily with a polypeptide having the modification than with a polypeptide not having the modification: (1) steric complementarity (also referred to as “steric hindrance”), (2) disulfide linkage (also referred to as “disulfide bond”), and (3) electrostatic charge (also referred to as “charge”).

In one embodiment, a modification introduced into a polypeptide of the present disclosure causes (1) steric complementarity (also referred to as “steric hindrance”), (2) disulfide linkage (also referred to as “disulfide bond”), or (3) electrostatic charge (also referred to as “charge”).

### Steric Complementarity (Steric Hindrance Control)

In one embodiment, a modification that causes steric complementarity (steric hindrance) according to the present disclosure includes modifying an amino acid residue(s) present in a region where association between polypeptides occurs (e.g., CH3 interface) to a bulky amino acid and/or a small amino acid. In one embodiment, modifications to a bulky amino acid and a small amino acid are carried out concurrently. In one embodiment, such a modification includes replacing an original small amino acid or non-bulky amino acid prior to modification with a bulky amino acid, or replacing an original bulky amino acid or non-bulky amino acid with a small amino acid.

In one embodiment, “bulky amino acids” include those with a larger molecular weight than the amino acid prior to modification. Bulky amino acids include, for example, tyrosine (Y), tryptophan (W), arginine (R), histidine (H), phenylalanine (F), leucine (L), valine (V), isoleucine (I), methionine (M), serine(S), threonine (T), cysteine (C), asparagine (N), glutamine (Q), lysine (K), aspartic acid (D), and glutamic acid (E).

In one embodiment, “small amino acids” include those with a smaller molecular weight than the amino acid prior to modification. Smaller amino acids include, for example, alanine (A), threonine (T), leucine (L), valine (V), asparagine (N), serine(S), isoleucine (I), methionine (M), glycine (G), cysteine (C), glutamine (Q), lysine (K), arginine (R), histidine (H), aspartic acid (D), and glutamic acid (E).

In one embodiment, a modification introduced into a polypeptide of the present disclosure causes steric complementarity (steric hindrance). In one embodiment, steric complementarity results from introducing knob-into-hole into a polypeptide of the present disclosure.

In one embodiment, said modification is a so-called “knob-into-hole” modification, comprising a protuberance (“knob”) modification and a cavity (“hole”) modification in an Fc region. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Protuberances (knobs) are constructed, for example, by replacing small amino acid side chains from the interface of a polypeptide of the present disclosure with larger side chains (e.g. tyrosine or tryptophan). Compensatory cavities (holes) of identical or similar size to the protuberances are created in the interface of the polypeptide, for example, by replacing large amino acid side chains with smaller ones (e.g. alanine or threonine).

In one embodiment, steric complementarity results from the presence of a knob and a hole in an Fc region. In one embodiment, a modification introduced into a polypeptide of the present disclosure is introduction of a knob and a hole into the Fc region. The knob and hole in the Fc region induces or promotes the homodimer formation of the polypeptide of the present disclosure.

In one embodiment, both a knob and a hole are introduced into the same molecule of a polypeptide of the present disclosure.

In one embodiment, in the CH3 domain of the Fc region of a polypeptide, an amino acid residue is replaced with an amino acid residue with a larger side-chain volume (knob construction), and another amino acid position in the same polypeptide is replaced with an amino acid residue with a smaller side-chain volume to create a cavity (hole construction).

The protuberance and cavity can be made by altering the nucleic acid encoding the polypeptides, e.g. by site-specific mutagenesis, or by peptide synthesis.

In one embodiment, amino acid positions to be modified for steric complementarity (steric hindrance control) include, for example, the following positions on the Fc region:
positions 347, 349, 350, 351, 354, 357, 364, 366, 368, 370, 392, 394, 399, 405, 407, and 409 (EU numbering).

In one embodiment, amino acid modifications for steric complementarity (steric hindrance control) include, for example, the following modifications (amino acid substitutions): Q347E, T350V, L351Y, S354Y, Y349T, E357N, S364H, T366W, T366Y, T366V, T366L, L368A, K370E, K392L, T394W, T394F, D399V, D399M, F405A, F405L, F405T, Y407V, Y407T, Y407A, K409R, K409W, and K409V.

In one embodiment, one or more of the above-mentioned modifications are combined.

In one embodiment, combinations of amino acid modifications for steric complementarity (steric hindrance control) include, for example, the following: T394W/F405A (“/” means that the preceding and following modifications are all included; the same applies hereinafter), T366W/L368A/Y407V, T366Y/Y407T, T366W/Y407A, T366Y/T394W/F405A/Y407T, E357N/K370E/D399V/F405T/K409W, Y349T/S364H/T394F/F405A, T394F/F405A, Q347E/S354Y/T366Y/Y407T, T350V/L351Y/T366L/T394W/F405A, K392L/T394W/F405A/Y407V, and T350V/L351Y/T366L/K392L/T394W/F405A/Y407V.

### Disulfide Linkage (Disulfide Bond Control)

In one embodiment, a modification introduced into a polypeptide of the present disclosure causes a disulfide linkage (disulfide bond) between modified polypeptides.

Modifications to introduce a disulfide linkage in a polypeptide of the present disclosure include, for example, substituting an amino acid other than cysteine with cysteine.

In one embodiment, the modification is a modification to introduce one or more cysteines into an Fc region. In one embodiment, the modification is substitution of one or more amino acids in an Fc region with cysteine.

In one embodiment, a disulfide linkage is formed between modified polypeptides, thereby stabilizing the homodimer.

In one embodiment, at least one amino acid substitution with a cysteine residue is introduced into a polypeptide comprising an Fc region, and a disulfide linkage is formed between the introduced cysteine and an existing cysteine.

In one embodiment, at least two amino acid substitutions with a cysteine residue are introduced into a polypeptide comprising an Fc region, and a disulfide linkage is formed between the introduced cysteine residues.

In one embodiment, a disulfide linkage is formed between polypeptides of the present disclosure.

In one embodiment, a disulfide linkage is formed between a modified (introduced) cysteine in one of polypeptides forming a homodimer and a cysteine at the corresponding same position in the other polypeptide.

In one embodiment, a disulfide linkage is formed between a modified (introduced) cysteine in one of polypeptides forming a homodimer and a cysteine at a different position in the other polypeptide.

In one embodiment, positions for amino acid modification to (substitution with) cysteine for disulfide linkage (disulfide bond control) include, for example, the following positions on the Fc region:
349, 351, 354, 356, 357, 392, 394, 397, and 399 (EU numbering).

In one embodiment, modifications (substitutions with cysteine) at one or more of the above-mentioned positions are combined.

In one embodiment, combinations of amino acid modifications to cysteine for disulfide linkage (disulfide bond control) include, for example, the following: K392C/D399C, Y349C/E356C, Y349C/E357C, L351C/S354C, and T394C/V397C.

In one embodiment, these modifications are combined.

In one embodiment, combined modifications (substitutions with cysteine) include, for example, the following:
Y349C/E357C/K392C/D399C, Y349C/L351C/S354C/E357C, Y349C/E357C/T394C/V397C, Y349C/E357C/K392C/T394C/V397C/D399C, K392C/T394C/V397C/D399C, and L351C/S354C/K392C/D399C.

### Electrostatic Charge (Charge Control)

In one embodiment, a modification introduced into a polypeptide of the present disclosure is introduction of a charged amino acid. In one embodiment, such modifications include substituting an amino acid residue in a polypeptide of the present disclosure with a charged amino acid residue.

In one embodiment, the action of electrostatic charge results from one or more charged amino acids modified (introduced) in an Fc region.

In one embodiment, “charged amino acid residues” (charged amino acids) are selected, for example, from amino acid residues included in either one of the following groups:
(a) glutamic acid (E) and aspartic acid (D); and
(b) lysine (K), arginine (R), and histidine (H).

In one embodiment, a modification introduced into a polypeptide of the present disclosure is introduction of two or more oppositely charged amino acids.

The phrase “oppositely charged” means, for example, that when at least one of the two or more amino acid residues is selected from the amino acid residues included in either one of groups (a) and (b) mentioned above, the remaining amino acid residues are selected from the amino acid residues included in the other group.

In one embodiment, a modification in a polypeptide of the present disclosure introduces a positively charged amino acid and/or a negatively charged amino acid.

In general, lysine (K), arginine (R), and histidine (H) are known as amino acids with a positive charge (positively charged amino acids). Glutamic acid (E) and aspartic acid (D) are known as amino acids with a negative charge (negatively charged amino acids).

In one embodiment, the above-mentioned positively charged amino acid is selected from lysine (K), arginine (R), and histidine (H), and/or the above-mentioned negatively charged amino acid is selected from aspartic acid (D) and glutamic acid (E).

In one embodiment, amino acid positions to be modified for electrostatic charge (charge control) include, for example, the following positions on the Fc region:
positions 345, 347, 351, 356, 357, 360, 366, 370, 392, 399, 409, and 439 (EU numbering).

In one embodiment, modifications at one or more of the above-mentioned positions are combined.

In one embodiment, positions to be modified to a positively charged amino acid include, for example, the following:
positions 345, 347, 351, 356, 357, 366, and 399.

In one embodiment, positions to be modified to a negatively charged amino acid include, for example, the following:
positions 351, 360, 370, 392, 409, and 439.

In one embodiment, combinations of amino acid positions to be modified to a positively charged amino acid or a negatively charged amino acid include, for example, the following:
modification of position 345 to a positively charged amino acid and modification of position 360 to a negatively charged amino acid;
modification of position 347 to a positively charged amino acid and modification of position 360 to a negatively charged amino acid;
modification of position 357 to a positively charged amino acid and modification of position 370 to a negatively charged amino acid;
modification of position 399 to a positively charged amino acid and modification of position 409 to a negatively charged amino acid;
modification of position 399 to a positively charged amino acid and modification of position 392 to a negatively charged amino acid;
modification of position 366 to a positively charged amino acid and modification of position 351 to a negatively charged amino acid;
modification of position 351 to a positively charged amino acid and modification of position 366 to a negatively charged amino acid; and
modification of position 356 to a positively charged amino acid and modification of position 439 to a negatively charged amino acid.

In one embodiment, one or more of the above-mentioned modifications are combined. In one embodiment, the above-mentioned modifications may be combined with a further modification to a positively or negatively charged amino acid alone.

Combined modifications include, for example, the following:
“modification of position 357 to a positively charged amino acid and modification of position 370 to a negatively charged amino acid” and “modification of position 399 to a positively charged amino acid and modification of position 409 to a negatively charged amino acid”;
“modification of position 356 to a positively charged amino acid and modification of position 439 to a negatively charged amino acid” and “modification of position 399 to a positively charged amino acid and modification of position 409 to a negatively charged amino acid”;
“modification of position 356 to a positively charged amino acid and modification of position 439 to a negatively charged amino acid” and “modification of position 357 to a positively charged amino acid and modification of position 370 to a negatively charged amino acid”;
“modification of position 356 to a positively charged amino acid and modification of position 439 to a negatively charged amino acid” and “modification of position 399 to a positively charged amino acid and modification of position 392 to a negatively charged amino acid”;
“modification of position 357 to a positively charged amino acid and modification of position 370 to a negatively charged amino acid” and “modification of position 399 to a positively charged amino acid and modification of position 392 to a negatively charged amino acid”;
“modification of position 399 to a positively charged amino acid and modification of position 409 to a negatively charged amino acid” and “modification of position 392 to a negatively charged amino acid”;
“modification of position 399 to a positively charged amino acid and modification of position 409 to a negatively charged amino acid”, “modification of position 392 to a negatively charged amino acid”, and “modification of position 356 to a positively charged amino acid”; and
“modification of position 356 to a positively charged amino acid and modification of position 439 to a negatively charged amino acid”, “modification of position 357 to a positively charged amino acid and modification of position 370 to a negatively charged amino acid”, and “modification of position 399 to a positively charged amino acid and modification of position 409 to a negatively charged amino acid”.

In one embodiment, amino acid modifications for electrostatic charge (charge control) include, for example, the following modifications:
E345K/K360E, E345R/K360E, Q347R/K360D, Q347R/K360E, E357K/K370E, D399K/K409E, D399K/K409D, D399R/K409E, D399R/K409D, K392E/D399K, K392D/D399K, K392E/D399R, K392D/D399R, L351D/T366K, L351E/T366K, L351K/T366D, L351K/T366E, and E356K/K439E.

In one embodiment, two or more of these modification can be combined. Examples include the following:
E357K/K370E/D399K/K409E, E356K/D399K/K409E/K439E, E356K/E357K/K370E/K439E, E356K/K392D/D399K/K439E, E357K/K370E/K392D/D399K, K392D/D399K/K409D, E356K/K392D/D399K/K409D, and E356K/E357K/K370E/D399K/K409E/K439E.

### Combinations of Introduced Modifications

In one embodiment, modifications introduced into a polypeptide of the present disclosure may be a combination of two or more different types of modifications.

In one embodiment, the combination of two or more different types of modifications is a combination selected from the aforementioned (1) steric complementarity, (2) disulfide linkage, and (3) electrostatic charge.

In one embodiment, the combination is a combination of (1) steric complementarity and (2) disulfide linkage, a combination of (1) steric complementarity and (3) electrostatic charge, or a combination of (2) disulfide linkage and (3) electrostatic charge.

In one embodiment, modifications introduced into a polypeptide of the present disclosure include those to introduce all of (1) steric complementarity, (2) disulfide linkage, and (3) electrostatic charge.

In the case of combining (1) steric complementarity and (3) electrostatic charge, in one embodiment, examples of modifications include, but are not limited to, the following: Q347R/K360E/D399V/F405T/K409W, E345R/Q347R/K360D/T366V/D399M/Y407A/K409V, and E345R/K360E/D399M/Y407A/K409V.

In the case of combining (2) disulfide linkage and (3) electrostatic charge, in one embodiment, examples of modifications include, but are not limited to, the following: Y349C/E357C/K392D/D399K, E357K/K370E/T394C/V397C, Y349C/E357C/K370E, E357K/K370E/K392C/D399C, and K392D/T394C/V397C/D399K.

In one embodiment, different modifications can be introduced into each of a plurality of polypeptides of the present disclosure to control the association of each polypeptide (in one embodiment, promote the homodimer formation of each polypeptide).

In one embodiment, a modification in a polypeptide of the present disclosure promotes the homodimer formation of the polypeptide. Amino acid modifications exhibiting strong homodimer formation-promoting ability include, for example, the following, but are not limited thereto (categories of modification effect shown in parentheses):
T394W/F405A (steric hindrance)
T366W/L368A/Y407V (steric hindrance)
Q347R/K360E/D399V/F405T/K409W (steric hindrance+charge)
T394F/F405A (steric hindrance)
E356K/D399K/K409E/K439E (charge)
Q347E/S354Y/T366Y/Y407T (steric hindrance)
K392L/T394W/F405A/Y407V (steric hindrance)
E356K/K392D/D399K/K439E (charge)
K392D/D399K/K409D (charge)
Y349C/E357C/K392D/D399K (charge+disulfide bond)
E345R/Q347R/K360D/T366V/D399M/Y407A/K409V (steric hindrance+charge)
E356K/K392D/D399K/K409D (charge)
E345R/K360E/D399M/Y407A/K409V (steric hindrance+charge)
T350V/L351Y/T366L/K392L/T394W/F405A/Y407V (steric hindrance).

Additionally, in one embodiment, such modifications include those shown in Tables 16-18. In one embodiment, the amino acid modifications disclosed herein can also be combined. Examples include the combinations of amino acid modifications shown in Tables 19-21.

In one embodiment, modifications introduced into a plurality of polypeptides of the present disclosure are selected from the above-mentioned (1) steric complementarity, (2) disulfide linkage, and (3) electrostatic charge. In one embodiment, when a modification for (1) steric complementarity, (2) disulfide linkage, or (3) electrostatic charge is introduced into a first polypeptide, a modification different from the one in the first polypeptide is introduced into a second polypeptide.

In one embodiment, combinations of modifications introduced into the first and second polypeptides include the combinations in Table 5 that show an amount of heterodimer of less than 52%.

In one embodiment, those combinations of modifications can be used when two antibodies are coexpressed. As shown in Example 6, pairs of modifications in such a relation as to suppress heterodimers in experimental results are expected not to cause heteromeric association no matter how many of them are combined. Therefore, such pairs can be used for not only coexpressing two antibodies but also coexpressing three, four, five or more antibodies.

### Modification of Amino Acid Residues

A “modification” of an amino acid residue in the present disclosure specifically refers to substitution of the original amino acid residue with another amino acid residue, deletion of the original amino acid residue, addition of a new amino acid residue, and such. In one embodiment, the modification refers to substitution of the original amino acid residue with another amino acid residue.

Modifications introduced into a polypeptide of the present disclosure are not limited to only the modifications for the above-mentioned (1) steric complementarity, (2) disulfide linkage, and (3) electrostatic charge, but, in one embodiment, may further include modifications other than these modifications.

In one embodiment, when a polypeptide of the present disclosure is an antibody, the constant regions of the antibody, in particular the heavy chain constant region, may be modified as necessary in order to improve antibody functions and/or stability. Examples of modifications to improve antibody functions include modifications to enhance or attenuate the binding of the antibody with an Fcγ receptor (FcγR), modifications to enhance or attenuate the binding of the antibody with an FcRn, and modifications to enhance or attenuate the cytotoxic activity of the antibody (e.g., ADCC activity, CDC activity, and such). Moreover, in one embodiment, modifications to improve the heterogeneity of the antibody and/or modifications to improve the immunogenicity and/or pharmacokinetics may be included. Moreover, in one embodiment, modifications to promote the hexamerization of the antibody and modifications to glycosylation sequences may be included.

For the heterogeneity of the heavy-chain C-terminal sequence of IgG antibody, deletion of the C-terminal lysine residue, and amidation of the C-terminal carboxyl group due to deletion of both of the C-terminal two amino acids, glycine and lysine, have been reported (Anal Biochem. 2007 Jan. 1; 360(1):75-83). Accordingly, when a polypeptide of the present disclosure is an antibody, in one embodiment, IgG with the C-terminal lysine or C-terminal lysine and glycine deleted can be used to reduce the C-terminal heterogeneity of the heavy chain Fc region.

### Interface

In one embodiment, the above-mentioned modifications are introduced into a region of the polypeptide that forms an interface. In one embodiment, the above-mentioned modifications are introduced into a region of the polypeptide comprising an Fc region that forms an interface in the Fc region. In one embodiment, the modifications are introduced into a region that forms the interface of CH3.

An “interface” in the present disclosure usually refers to a surface at which association (interaction) occurs. An amino acid residue(s) forming an interface usually refers to one or more amino acid residues included in a polypeptide region subjected to that association, and, in one embodiment, refers to amino acid residue(s) that come close and are involved in interaction when association occurs.

In the present disclosure, the amino acid residues in a polypeptide subjected to modification are not limited to those in the Fc region or CH3 region. In one embodiment, amino acid residues are modified in a region that forms an interface between polypeptides. Those skilled in the art can identify the amino acid residues that form an interface by homology modeling and such using commercially available software. Then, amino acid residues of these positions can be subjected to modification so as to control the association.

In one embodiment, the amino acid residues to be modified are those that come close to each other when association occurs between polypeptide regions forming an interface.

### Antibody Formats Subjected to Modification

In one embodiment, an antibody format to be subjected to a modification of the present disclosure is an IgG format. For example, wild-type IgG, which has no modification in Fc, or IgG containing a modification in Fc, is used. In one embodiment, various antibody formats such as a VHH or scFv linked with an Fc, and antibody formats into which an amino acid modification for changing an Fc function has been introduced can be used. In one embodiment, IgG formats having a deglycosylated Fc region and/or containing modifications for enhancing the binding to an FcRn, suppressing the binding to an FcγR, and/or promoting hexamer formation may be used. In one embodiment, Ig formats containing an Fc that is linked with a polypeptide containing a Fab, VHH, or scFv, or with a polypeptide other than Fab, VHH, or scFv, can also be used. In one embodiment, IgG formats containing an amino acid modification that does not affect the Fc interface can be used.

### Coexpression of Homodimer and Heteromultimer

In one embodiment, an amino acid modification for promoting homodimer formation of the present disclosure can be used concurrently with an amino acid modification for promoting heterodimer formation (e.g., a knob-into-hole modification).

In one embodiment, the present disclosure relates to a method for coexpressing a homodimer and a heteromultimer.

In one embodiment, the method comprises:
the step of obtaining a nucleic acid encoding a first polypeptide;
the step of obtaining a nucleic acid encoding a second polypeptide;
the step of obtaining a nucleic acid encoding a third polypeptide; and/or
the step of expressing said nucleic acids.

Here, in one embodiment, the first polypeptide has an Fc region into which a modification has been introduced. Because of the modification introduced into the Fc region, the first polypeptide associates more readily with a first polypeptide having that modification via the Fc region than with a first polypeptide comprising an Fc region into which the modification has not been introduced.

Here, in one embodiment, the second polypeptide associates more readily with the third polypeptide than with the second polypeptide.

Here, in one embodiment, the second polypeptide has an Fc region into which a modification has been introduced. Because of the modification introduced into the Fc region, the second polypeptide associates more readily with the third polypeptide via the Fc region than with the second polypeptide.

In one embodiment, amino acid modifications for promoting heterodimer formation include the modifications shown in Example 13 described later.

### Combinations of Modifications Exhibiting Strong Suppression of Heteromeric Association

In one embodiment, the above-mentioned Fc region into which a modification has been introduced includes Fc regions into which a modification exhibiting strong suppression of heteromeric association (e.g., a combination of modifications) has been introduced. Combinations of modifications that exhibit strong suppression of heteromeric association include, for example, the amino acid modifications shown in Tables 19-21.

In one embodiment, the above-mentioned Fc region into which a modification has been introduced contains amino acid modifications at one combination or two or more combinations of positions selected from the combinations of positions shown in (a) to (d) below according to EU numbering:
(a) positions 394 and 405;
(b) positions 366, 368, and 407;
(c) positions 347, 360, 399, 405 and 409; and
(d) positions 356, 392, 399 and 439.

In one embodiment, the above-mentioned Fc region into which a modification has been introduced contains at least one amino acid selected from the group consisting of:
(a) W, F, or Y at position 394, and
   A, S, T, C, G, or V at position 405;
(b) W, Y, or F at position 366,
   A, S, T, C, V, or G at position 368, and
   V, L, I, M, A, S, T, C, N, or Q at position 407;
(c) R, K, Y, or H at position 347,
   E or D at position 360,
   V, L, I, M, S, T, C, H, A, N, Q, or G at position 399,
   T, A, V, S, C, N, D, or G at position 405, and
   W, F, Y, or H at position 409; and
(d) K or R at position 356,
   D or E at position 392,
   K or R at position 399, and
   E or D at position 439
according to EU numbering.

In one embodiment, the above-mentioned Fc region into which a modification has been introduced contains at least one amino acid selected from the group consisting of:
(a) W or F at position 394, and
   A, S, T, or G at position 405;
(b) W, Y, or F at position 366,
   A, T, C, V, or G at position 368, and
   V, L, I, M, A, or C at position 407;
(c) R, K, Y, or H at position 347,
   E or D at position 360,
   V, L, I, M, S, T, C, H, A, N, or G at position 399,
   T, A, or V at position 405, and
   W or F at position 409; and
(d) K at position 356,
   D at position 392,
   K at position 399, and
   E at position 439,
according to EU numbering.

In one embodiment, the above-mentioned Fc region into which a modification has been introduced contains amino acid modifications at one combination or two or more combinations of positions selected from the combinations of positions shown in (a) to (d) below according to EU numbering:
(a) positions 345, 347, 360, 366, 399, 407, and 409;
(b) positions 356, 399, 409, and 439;
(c) positions 356, 392, 399, and 409; and
(d) positions 392, 399, and 409.

In one embodiment, the above-mentioned Fc region into which a modification has been introduced contains at least one amino acid selected from the group consisting of:
(a) R or E at position 345,
   R or K at position 347,
   D or E at position 360,
   V at position 366,
   M, Q, N, H, I, F, Y, T, S, V, or L at position 399,
   A at position 407, and
   V, Q, N, H, L, I, F, Y, T, or S at position 409;
(b) K, R, or H at position 356,
   K, R, or H at position 399,
   E or D at position 409, and
   E or D at position 439,
(c) K, R, or H at position 356,
   D or E at position 392,
   K, R, or H at position 399,
   D or E at position 409; and
(d) D or E at position 392,
   K or R at position 399, and
   D or E at position 409,
according to EU numbering.

### Purification of Polypeptides

Polypeptides (in one embodiment, antibodies) prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the polypeptide binds. For example, for affinity chromatography purification of polypeptides (e.g., antibodies) of the present disclosure, a matrix with protein A or protein G may be used. Sequential Protein A or G affinity chromatography and size exclusion chromatography can be used to isolate a polypeptide. The purity of a polypeptide of the present disclosure can be determined by any of a variety of well-known analytical methods including gel electrophoresis, high pressure liquid chromatography, and the like.

### Compositions

In one embodiment, the present disclosure relates to a composition comprising a nucleic acid encoding a polypeptide of the present disclosure.

In one embodiment, the composition comprises a nucleic acid encoding a first polypeptide, wherein the first polypeptide has an Fc region into which a modification has been introduced, and wherein, because of the modification introduced into the Fc region, the first polypeptide associates more readily with a polypeptide having the modification than with a polypeptide not having the modification.

In one embodiment, the composition of the present disclosure comprises a nucleic acid encoding a second polypeptide, wherein the second polypeptide has an Fc region into which a modification has been introduced, and wherein, because of the modification introduced into the Fc region, the second polypeptide associates more readily with a polypeptide having the modification than with a polypeptide not having the modification.

In one embodiment, the composition of the present disclosure may further comprise a nucleic acid(s) encoding multiple different polypeptides different from the above-mentioned first or second polypeptide (in one embodiment, a third and/or a fourth polypeptide).

In one embodiment, a composition of the present disclosure comprises:
a nucleic acid encoding a first polypeptide;
a nucleic acid encoding a second polypeptide; and
a nucleic acid encoding a third polypeptide.

Here,
in one embodiment, the first polypeptide has an Fc region into which a modification has been introduced, and because of the modification introduced into the Fc region, the first polypeptide associates more readily with a first polypeptide having the modification via the Fc region than with a first polypeptide comprising an Fc region into which the modification has not been introduced.

In one embodiment, the second polypeptide associates more readily with the third polypeptide than with the second polypeptide.

In one embodiment, the second polypeptide has an Fc region into which a modification has been introduced, and because of the modification introduced into the Fc region, the second polypeptide associates more readily with the third polypeptide via the Fc region than with the second polypeptide.

### Pharmaceutical Compositions

In one embodiment, the present disclosure provides a pharmaceutical composition comprising a nucleic acid or polypeptide (in one embodiment, an antibody) of the disclosure. In one embodiment, the present disclosure relates to a composition (pharmaceutical composition) comprising a nucleic acid or polypeptide of the disclosure and a pharmaceutically acceptable carrier.

In one embodiment, a pharmaceutical formulation of the present disclosure is prepared by mixing a polypeptide (in one embodiment, an antibody) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

A polypeptide (in one embodiment, an antibody), a pharmaceutical composition, or such of the present disclosure is, for example, either orally or parenterally administered to a subject (in one embodiment, a patient). For example, parenteral administration is preferred. Specifically, such administration methods include injection, nasal administration, transpulmonary administration, and percutaneous administration. Injections include, for example, intravenous injections, intramuscular injections, intraperitoneal injections, and subcutaneous injections. For example, pharmaceutical compositions of the present disclosure can be administered locally or systemically by injection. Furthermore, appropriate administration methods can be selected according to the subject's (patient's) age and symptoms. The administered dose can be selected, for example, from the range of 0.0001 mg to 1,000 mg per kg of body weight for each administration. Alternatively, the dose can be selected, for example, from the range of 0.001 mg/body to 100,000 mg/body per patient. However, the dose of a pharmaceutical composition of the present disclosure is not limited to these doses.

In one embodiment, a pharmaceutical composition of the present disclosure comprises a nucleic acid of the disclosure. In one embodiment, the pharmaceutical composition comprises a nucleic acid of the disclosure included (encapsulated) in a vesicle. Such vesicles include, for example, lipid nanoparticles (LNPs), viruses, extracellular vesicles (EVs), and liposomes.

### Direct Expression in Subjects

If necessary, a vector comprising a nucleic acid molecule encoding a polypeptide (in one embodiment, an antibody) of the present disclosure may be introduced into a subject to express the polypeptide of the present disclosure directly within the subject. Subjects include, for example, a human, a non-human animal, an ex vivo cell, and an in vitro cell. An example of the vector that can be used is adenovirus, but is not limited thereto. It is also possible to administer a nucleic acid molecule encoding a polypeptide (in one embodiment, an antibody) of the present disclosure directly into a subject, to administer the nucleic acid molecule included (encapsulated) in a vesicle, to transfer a nucleic acid molecule encoding a polypeptide of the present disclosure via electroporation to a subject, or to administer cells comprising a nucleic acid molecule encoding a polypeptide of the present disclosure to be expressed into a subject to express the polypeptide (in one embodiment, antibody) of the present disclosure in the subject.

In one embodiment, the present disclosure relates to expression of a polypeptide for which association between Fc regions is controlled, in a living organism. Polypeptides of the present disclosure are, in one embodiment, antibodies that do not associate with wild-type IgG, thus making it possible to provide therapeutic drugs based on homogeneous, safe, multiple antibodies when antibody expression in living organisms is intended. In one embodiment, in the case of expressing therapeutic antibodies in living organisms, their not associating with endogenous IgG will lead to reduced side effects.

In one embodiment, a messenger RNA (mRNA) encoding a polypeptide of the present disclosure can be introduced into a subject (a mammal, a human, a non-human animal, or such) to promote homodimer formation of the polypeptide of the present disclosure.

In one embodiment, the introduction into a subject can be performed using mRNA-encapsulated lipid nanoparticles (mRNA-LNPs).

Method for Obtaining Polypeptides for which Association is Controlled

In one embodiment, the present disclosure relates to a method for obtaining a polypeptide for which association between polypeptides is controlled.

In one embodiment, the method comprises:
the step of obtaining a nucleic acid encoding the polypeptide; and
the step of expressing the nucleic acid.

In one embodiment, the above-mentioned polypeptide has an Fc region, and because of the modification introduced into the Fc region, the polypeptide associates more readily with a polypeptide having the modification than with a polypeptide not having the modification.

In one embodiment, the above-mentioned expression is in vivo, ex vivo, or in vitro expression.

Herein, the term “step” is used interchangeably with “process” and “phase”.

### Method for Production and Method for Controlling Association

In one embodiment, the present disclosure relates to a method for producing a polypeptide for which association is controlled. In one embodiment, the production method is a method for producing a polypeptide for which association of the Fc region is controlled.

In one embodiment, the production method comprises the following steps:
(a) obtaining a nucleic acid encoding a polypeptide into which a modification has been introduced in the Fc region;
(b) introducing the nucleic acid into a host cell and culturing the host cell to express the nucleic acid; and
(c) recovering the polypeptide from the culture of the host cell.

In one embodiment, the above-mentioned polypeptide associates more readily with a polypeptide having the modification than with a polypeptide not having the modification due to at least one of the following actions: (1) steric complementarity, (2) disulfide linkage, and (3) electrostatic charge.

In one embodiment, the above-mentioned production method further comprises the step of introducing a modification into the Fc region such that the action of (1) steric complementarity, (2) disulfide linkage, and/or (3) electrostatic charge occurs in the above-mentioned polypeptide.

In one embodiment, the production method comprises the following steps:
(a) modifying a nucleic acid encoding a polypeptide comprising an Fc region such that the polypeptide associates more readily with a polypeptide having the modification than with a polypeptide not having the modification due to at least one of the following actions: (1) steric complementarity, (2) disulfide linkage, and (3) electrostatic charge;
(b) introducing the modified nucleic acid into a host cell and culturing the host cell to express the nucleic acid; and
(c) recovering the polypeptide from the culture of the host cell.

In one embodiment, the present disclosure relates to a method for controlling association between polypeptides.

In one embodiment, the method is a method for controlling association between polypeptides comprising an Fc region. In one embodiment, the method is a method for controlling association between CH3 domains.

In one embodiment, the method comprises modifying a polypeptide such that it associates more readily with a polypeptide having the modification than with a polypeptide not having the modification due to at least one of the following actions: (1) steric complementarity, (2) disulfide linkage, and (3) electrostatic charge.

In one embodiment, a method for controlling the association of the homomer of a polypeptide comprises:
the step of obtaining a nucleic acid encoding the polypeptide; and
the step of expressing the nucleic acid,
wherein the polypeptide has an Fc region, and
wherein, because of the modification introduced into the Fc region, the polypeptide associates more readily with a polypeptide having the modification via the Fc region than with a polypeptide comprising an Fc region into which the modification has not been introduced.

In one embodiment, the present disclosure relates to a method for promoting the expression of a homomer of a polypeptide.

In one embodiment, the method comprises the step of obtaining a nucleic acid encoding the polypeptide; and
the step of expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

All prior art documents cited herein are incorporated herein by reference.

The Examples provided below are one embodiment of the present invention, and the present invention is not limited to these embodiments. In the Sequence Listing, SEQ ID NOs: 1-60, 78-101, and 104-114 are full-length heavy chains, SEQ ID NO: 61 is a light chain, and SEQ ID NOs: 62-77 and 102-103 are Fc fragments. (For SEQ ID NOs: 115-620, see Table 14 and subsequent tables.)

The value “0” in the tables presented in the Examples is construed as having the same significant figures as the other values in the same column. For example, when the other values in the same column have significant figures to one decimal place, “0” is construed as “0.0”.

### EXAMPLES

### [Example 1] Search for Amino Acid Modifications for Promoting Homodimer Formation of Heavy Chains

To express multiple antibodies simultaneously in cells, first, a search was performed for amino acid modifications positioned in the CH3 interface that are disadvantageous for heterodimer formation between the heavy chains of two antibodies in terms of electrostatic force, disulfide linkage formation, or steric hindrance, but are advantageous for homodimer formation. The concept is shown in FIG. 1. A number of amino acid modifications for the purpose of promoting heterodimerization of the heavy chains of antibodies A and B have so far been reported (listed below). Accordingly, it was considered that similar residues might also be able to be used as residues for promoting homodimerization.
WO2006106905—PROCESS FOR PRODUCTION OF POLYPEPTIDE BY REGULATION OF ASSEMBLY
An efficient route to human bispecific IgG (Nat Biotechnol. 1998 July; 16(7):677-81. doi: 10.1038/nbt0798-677.)
Efficient generation of stable bispecific IgG1 by controlled Fab-arm exchange (Proc Natl Acad Sci USA. 2013 Mar. 26; 110(13):5145-50. doi: 10.1073/pnas. 1220145110.)
Immunoglobulin Fc Heterodimer Platform Technology: From Design to Applications in
Therapeutic Antibodies and Proteins (Front Immunol. 2016 Oct. 6; 7:394. doi: 10.3389/fimmu.2016.00394. eCollection 2016.)

However, since the modification for promoting the homodimer formation of heavy chain needs to be achieved between heavy chains of one type, the degree of freedom of available amino acid modifications is lower than when promoting heterodimer formation using two different heavy chains. In particular, the promotion of disulfide formation between identical heavy chains and the controlling method based on physical hindrance via protuberances and cavities are expected to be difficult because they require more stringent orientation than electrostatic interactions, which can take effect in a relatively long distance of about 5-10 Å, and therefore the modifications used for heterodimers may not be compatible. In fact, only limited combinations of charged amino acid modifications have so far been reported to promote homodimer formation (WO2013157953A1, WO2014015804A1, WO2017205014, J Biol Chem. 2017 Oct. 27; 292(43): 17885-17896). Under these circumstances, the present inventors carried out a large-scale study on combinations of amino acid modifications on the CH3 interface to search for amino acid modifications that promote homodimer formation. A list of the studied amino acid modifications and the intended effects of modification is shown in Table 1. The amino acid numbers in Table 1 are in accordance with EU numbering (Sequences of proteins of immunological interest, NIH Publication No. 91-3242). The subsequent amino acid numbers are also in accordance with EU numbering. In screening, the heavy chain used was a human IgG1 sequence.

Plasmids expressing a full-length heavy chain with these amino acid modifications and an Fc fragment below the hinge region containing no modification were prepared by a method known to those skilled in the art. When these two plasmids and a plasmid encoding a light chain are expressed simultaneously in mammalian cells, the homodimer of the full-length IgG heavy chain, the homodimer of the Fc fragment, and the heterodimer in which the two are associated, will be produced. The ratio of expression of these three can be analyzed by size exclusion chromatography (SEC) because they have different molecular weights. To confirm whether the formation of heavy chain homodimer was promoted, each peak area ratio was calculated and compared with unmodified (WT) human IgG1. If the formation of heavy chain homodimer is promoted, the area ratio of the heterodimer is expected to decrease as compared to WT-IgG1. The combinations of the genes of each antibody used in screening and their SEQ ID NOs are shown in Table 1.

The antibodies listed in Table 1 were transiently expressed in mammalian cells by a method known to those skilled in the art using the produced genes, and then purified by a method known to those skilled in the art. Specifically, the antibodies of Table 1 were transiently expressed by transfecting 1 mL of 2E+6 cells/mL Expi293F (Thermo Fisher Scientific) with the plasmids encoding the full-length heavy chain, the light chain, and the Fc fragment at a mass ratio of 1:2:1. After 4 days of transfection, the supernatant was recovered and purified using MonoSpin ProA (GL science).

The purified antibodies were assessed by size exclusion chromatography (SEC) analysis using ACQUITY UPLC H-Class (Waters). Fifty mM phosphate buffer containing 300 mM sodium chloride, pH7.0 (Isekyu) was used as a running buffer, and TSKgel SuperSW3000 custom-made column (4.6 mm×15 cm, 4 μm, Gel Lot 89R) (TOSOH) was used as an analysis column. Chromatograms were recorded at a wavelength of UV 215 nm. Data was analyzed using Empower3 (Waters). For analysis of the antibodies of Table 1, the area ratios of the three components, i.e., the homodimer component of the full-length heavy chain, the heterodimer component with the Fc fragment, and the homodimer component of the Fc fragment, were calculated separately and presented in percent as shown in the representative example of FIG. 2. In FIG. 2, the reference samples prepared in Sample Nos. 61 and 62 (Table 2) are shown as well, thereby confirming that the three components can be properly separated in SEC analysis. Analysis results are shown in Table 1. In this analysis, the ratio of the heteromultimer for WT-IgG1 was 51.7%, suggesting that amino acid modifications exhibiting this ratio or lower will promote homodimerization of heavy chains.

TABLE 1
Modifications and amino acid sequences used in screening and SEC analysis results

| | | | Full- | | | Homo | | |
|---|---|---|---|---|---|---|---|---|
| | | | length | | | full- | | |
| | | Modifications that | heavy | Light | Fc | length | | |
| | Category of | promote formation | chain | chain | fragment | heavy | Hetero- | Homo Fc |
| Sample | modification | of heavy chain | SEQ ID | SEQ ID | SEQ ID | chain | multimer | fragment |
| No. | effect | homodimers | NO | NO | NO | (%) | (%) | (%) |
| 1 | WT-IgG1 | — | 1 | 61 | 62 | 31.5 | 51.7 | 16.8 |
| 2 | Charge | E345K/K360E | 2 | 61 | 62 | 34.3 | 50.3 | 15.4 |
| 3 | Charge | E345R/K360E | 3 | 61 | 62 | 31.0 | 51.8 | 17.1 |
| 4 | Charge | E357K/K370E | 4 | 61 | 62 | 42.5 | 40.6 | 17.0 |
| 5 | Charge | D399K/K409E | 5 | 61 | 62 | 56.4 | 12.1 | 31.5 |
| 6 | Charge | D399K/K409D | 6 | 61 | 62 | 53.6 | 18.6 | 27.8 |
| 7 | Charge | D399R/K409E | 7 | 61 | 62 | 57.0 | 13.2 | 29.8 |
| 8 | Charge | D399R/K409D | 8 | 61 | 62 | 57.1 | 14.4 | 28.5 |
| 9 | Charge | K392E/D399K | 9 | 61 | 62 | 40.0 | 36.1 | 23.9 |
| 10 | Charge | K392D/D399K | 10 | 61 | 62 | 54.2 | 20.3 | 25.6 |
| 11 | Charge | K392E/D399R | 11 | 61 | 62 | 40.4 | 37.7 | 21.8 |
| 12 | Charge | K392D/D399R | 12 | 61 | 62 | 47.7 | 27.9 | 24.4 |
| 13 | Disulfide bond | K392C/D399C | 13 | 61 | 62 | 35.8 | 41.5 | 22.8 |
| 14 | Disulfide bond | Y349C/S354C | 14 | 61 | 62 | 27.6 | 52.9 | 19.5 |
| 15 | Disulfide bond | Y349C/E356C | 15 | 61 | 62 | 34.2 | 48.1 | 17.7 |
| 16 | Disulfide bond | Y349C/E357C | 16 | 61 | 62 | 36.2 | 41.5 | 22.3 |
| 17 | Disulfide bond | L351C/S354C | 17 | 61 | 62 | 27.4 | 49.4 | 23.2 |
| 18 | Disulfide bond | T394C/V397C | 18 | 61 | 62 | 33.1 | 46.8 | 20.1 |
| 19 | Steric hindrance | T394W/F405A | 19 | 61 | 62 | 70.7 | 0.0 | 29.3 |
| 20 | Charge | L351D/T366K | 20 | 61 | 62 | 66.8 | 1.0 | 32.2 |
| 21 | Charge | L351E/T366K | 21 | 61 | 62 | 50.8 | 12.1 | 37.1 |
| 22 | Charge | L351K/T366D | 22 | 61 | 62 | 55.0 | 16.2 | 28.8 |
| 23 | Charge | L351K/T366E | 23 | 61 | 62 | 39.6 | 38.0 | 22.4 |
| 24 | Steric hindrance + | F405L/K409R | 24 | 61 | 62 | 8.0 | 83.0 | 9.0 |
| | Charge | | | | | | | |
| 25 | Charge | E356K/K439E | 25 | 61 | 62 | 53.0 | 16.0 | 31.0 |
| 26 | Steric hindrance | T366W/L368A/Y407V | 26 | 61 | 62 | 64.0 | 0.0 | 36.0 |
| 27 | Steric hindrance | T366Y/Y407T | 27 | 61 | 62 | 65.5 | 3.3 | 31.2 |
| 28 | Steric hindrance | T366W/Y407A | 28 | 61 | 62 | 55.4 | 7.7 | 37.0 |
| 29 | Steric hindrance | T366Y/T394W/F405A/Y407T | 29 | 61 | 62 | 58.4 | 6.7 | 34.9 |
| 30 | Steric hindrance | E357N/K370E/D399V/F405T/K409W | 30 | 61 | 62 | 62.5 | 3.9 | 33.6 |
| 31 | Steric hindrance + | Q347R/K360E/D399V/F405T/K409W | 31 | 61 | 62 | 73.6 | 0.0 | 26.4 |
| | Charge | | | | | | | |
| 32 | Steric hindrance | Y349T/S364H/T394F/F405A | 32 | 61 | 62 | 30.7 | 26.5 | 42.8 |
| 33 | Steric hindrance | Y349T/S364H | 33 | 61 | 62 | 24.1 | 65.2 | 10.7 |
| 34 | Steric hindrance | T394F/F405A | 34 | 61 | 62 | 72.6 | 0.0 | 27.4 |
| 35 | Charge | E357K/K370E/D399K/K409E | 35 | 61 | 62 | 27.0 | 45.0 | 28.0 |
| 36 | Charge | E356K/D399K/K409E/K439E | 36 | 61 | 62 | 64.0 | 0.0 | 36.0 |
| 37 | Charge | E356K/E357K/K370E/K439E | 37 | 61 | 62 | 55.8 | 10.4 | 33.8 |
| 38 | Charge | E356K/E357K/K370E/D399K/K409E/K439E | 38 | 61 | 62 | 58.5 | 1.3 | 40.2 |
| 39 | Steric hindrance | Q347E/S354Y/T366Y/Y407T | 39 | 61 | 62 | 61.6 | 0.0 | 38.4 |
| 40 | Steric hindrance | T350V/L351Y/T366L/T394W/F405A | 40 | 61 | 62 | 54.2 | 8.6 | 37.3 |
| 41 | Steric hindrance | K392L/T394W/F405A/Y407V | 41 | 61 | 62 | 65.9 | 0.0 | 34.1 |
| 42 | Disulfide bond | Y349C/E357C/K392C/D399C | 42 | 61 | 62 | 22.8 | 7.2 | 70.1 |
| 43 | Disulfide bond | Y349C/L351C/S354C/E357C | 43 | 61 | 62 | 40.2 | 22.7 | 37.1 |
| 44 | Disulfide bond | Y349C/E357C/T394C/V397C | 44 | 61 | 62 | 20.0 | 23.1 | 56.9 |
| 45 | Disulfide bond | Y349C/E357C/K392C/T394C/V397C/D399C | 45 | 61 | 62 | 24.3 | 0.0 | 75.7 |
| 46 | Disulfide bond | K392C/T394C/V397C/D399C | 46 | 61 | 62 | 24.0 | 30.2 | 45.8 |
| 47 | Disulfide bond | L351C/S354C/K392C/D399C | 47 | 61 | 62 | 21.1 | 39.3 | 39.7 |
| 48 | Charge | E356K/K392D/D399K/K439E | 48 | 61 | 62 | 68.6 | 0.0 | 31.4 |
| 49 | Charge | E357K/K370E/K392D/D399K | 49 | 61 | 62 | 59.6 | 8.3 | 32.1 |
| 50 | Charge | K392D/D399K/K409D | 50 | 61 | 62 | 65.5 | 0.0 | 34.5 |
| 51 | Charge + | Y349C/E357C/K392D/D399K | 51 | 61 | 62 | 36.0 | 0.0 | 64.0 |
| | Disulfide bond | | | | | | | |
| 52 | Charge + | E357K/K370E/T394C/V397C | 52 | 61 | 62 | 38.8 | 28.4 | 32.8 |
| | Disulfide bond | | | | | | | |
| 53 | Charge + | Y349C/E357C/K370E | 53 | 61 | 62 | 39.8 | 31.3 | 28.9 |
| | Disulfide bond | | | | | | | |
| 54 | Charge + | E357K/K370E/K392C/D399C | 54 | 61 | 62 | 52.4 | 20.6 | 27.0 |
| | Disulfide bond | | | | | | | |
| 55 | Steric hindrance + | E345R/Q347R/K360D/T366V/D399M/Y407A/K409V | 55 | 61 | 62 | 67.9 | 0.0 | 32.1 |
| | Charge | | | | | | | |
| 56 | Charge | E356K/K392D/D399K/K409D | 56 | 61 | 62 | 62.9 | 0.0 | 37.1 |
| 57 | Steric hindrance + | E345R/Q347R/K360D/T366V | 57 | 61 | 62 | 11.5 | 70.8 | 17.7 |
| | Charge | | | | | | | |
| 58 | Steric hindrance + | E345R/K360E/D399M/Y407A/K409V | 58 | 61 | 62 | 63.1 | 0.0 | 36.9 |
| | Charge | | | | | | | |
| 59 | Charge + | K392D/T394C/V397C/D399K | 59 | 61 | 62 | 39.9 | 19.9 | 40.3 |
| | Disulfide bond | | | | | | | |
| 60 | Steric hindrance | T350V/L351Y/T366L/K392L/T394W/F405A/Y407V | 60 | 61 | 62 | 61.9 | 0.0 | 38.1 |

### [Example 2] Expression and Purification of Antibodies

Next, in order to examine the effect of these amino modifications in the CH3 interface on the physicochemical properties of antibody, the antibodies of Table 2 were prepared. The antibodies were transiently expressed in mammalian cells by the method described in Example 1 using genes produced by a method known to those skilled in the art, and then purified. The plasmids encoding the full length heavy chain and light chain were used for transfection at a mass ratio of 1:1 or 1:2.

TABLE 2
Modifications and SEQ ID NOs of antibodies used in analysis

| | | | Full- | |
|---|---|---|---|---|
| | | | length | |
| | | | heavy | Light |
| | | | chain | chain |
| Sample | Category of | Modifications that promote formation of heavy | SEQ | SEQ |
| No. | modification effect | chain homodimers | ID NO | ID NO |
| 61 | WT-IgG1 | — | 1 | 61 |
| 62 | WT-Fc fragment | — | 62 | — |
| 63 | Charge | E345K/K360E | 2 | 61 |
| 64 | Charge | E345R/K360E | 3 | 61 |
| 65 | Charge | E357K/K370E | 4 | 61 |
| 66 | Charge | D399K/K409E | 5 | 61 |
| 67 | Charge | D399K/K409D | 6 | 61 |
| 68 | Charge | D399R/K409E | 7 | 61 |
| 69 | Charge | D399R/K409D | 8 | 61 |
| 70 | Charge | K392E/D399K | 9 | 61 |
| 71 | Charge | K392D/D399K | 10 | 61 |
| 72 | Charge | K392E/D399R | 11 | 61 |
| 73 | Charge | K392D/D399R | 12 | 61 |
| 74 | Disulfide bond | K392C/D399C | 13 | 61 |
| 75 | Disulfide bond | Y349C/S354C | 14 | 61 |
| 76 | Disulfide bond | Y349C/E356C | 15 | 61 |
| 77 | Disulfide bond | Y349C/E357C | 16 | 61 |
| 78 | Disulfide bond | L351C/S354C | 17 | 61 |
| 79 | Disulfide bond | T394C/V397C | 18 | 61 |
| 80 | Steric hindrance | T394W/F405A | 19 | 61 |
| 81 | Charge | L351D/T366K | 20 | 61 |
| 82 | Charge | L351E/T366K | 21 | 61 |
| 83 | Charge | L351K/T366D | 22 | 61 |
| 84 | Charge | L351K/T366E | 23 | 61 |
| 85 | Steric hindrance + | F405L/K409R | 24 | 61 |
| | Charge | | | |
| 86 | Charge | E356K/K439E | 25 | 61 |
| 87 | Steric hindrance | T366W/L368A/Y407V | 26 | 61 |
| 88 | Steric hindrance | T366Y/Y407T | 27 | 61 |
| 89 | Steric hindrance | T366W/Y407A | 28 | 61 |
| 90 | Steric hindrance | T366Y/T394W/F405A/Y407T | 29 | 61 |
| 91 | Steric hindrance | E357N/K370E/D399V/F405T/K409W | 30 | 61 |
| 92 | Steric hindrance + | Q347R/K360E/D399V/F405T/K409W | 31 | 61 |
| | Charge | | | |
| 93 | Steric hindrance | Y349T/S364H/T394F/F405A | 32 | 61 |
| 94 | Steric hindrance | Y349T/S364H | 33 | 61 |
| 95 | Steric hindrance | T394F/F405A | 34 | 61 |
| 96 | Charge | E357K/K370E/D399K/K409E | 35 | 61 |
| 97 | Charge | E356K/D399K/K409E/K439E | 36 | 61 |
| 98 | Charge | E356K/E357K/K370E/K439E | 37 | 61 |
| 99 | Charge | E356K/E357K/K370E/D399K/K409E/K439E | 38 | 61 |
| 100 | Steric hindrance | Q347E/S354Y/T366Y/Y407T | 39 | 61 |
| 101 | Steric hindrance | T350V/L351Y/T366L/T394W/F405A | 40 | 61 |
| 102 | Steric hindrance | K392L/T394W/F405A/Y407V | 41 | 61 |
| 103 | Disulfide bond | Y349C/E357C/K392C/D399C | 42 | 61 |
| 104 | Disulfide bond | Y349C/L351C/S354C/E357C | 43 | 61 |
| 105 | Disulfide bond | Y349C/E357C/T394C/V397C | 44 | 61 |
| 106 | Disulfide bond | Y349C/E357C/K392C/T394C/V397C/D399C | 45 | 61 |
| 107 | Disulfide bond | K392C/T394C/V397C/D399C | 46 | 61 |
| 108 | Disulfide bond | L351C/S354C/K392C/D399C | 47 | 61 |
| 109 | Charge | E356K/K392D/D399K/K439E | 48 | 61 |
| 110 | Charge | E357K/K370E/K392D/D399K | 49 | 61 |
| 111 | Charge | K392D/D399K/K409D | 50 | 61 |
| 112 | Charge + Disulfide | Y349C/E357C/K392D/D399K | 51 | 61 |
| | bond | | | |
| 113 | Charge + Disulfide | E357K/K370E/T394C/V397C | 52 | 61 |
| | bond | | | |
| 114 | Charge + Disulfide | Y349C/E357C/K370E | 53 | 61 |
| | bond | | | |
| 115 | Charge + Disulfide | E357K/K370E/K392C/D399C | 54 | 61 |
| | bond | | | |
| 116 | Steric hindrance + | E345R/Q347R/K360D/T366V/D399M/Y407A/ | 55 | 61 |
| | Charge | K409V | | |
| 117 | Charge | E356K/K392D/D399K/K409D | 56 | 61 |
| 118 | Steric hindrance + | E345R/Q347R/K360D/T366V | 57 | 61 |
| | Charge | | | |
| 119 | Steric hindrance + | E345R/K360E/D399M/Y407A/K409V | 58 | 61 |
| | Charge | | | |
| 120 | Charge + Disulfide | K392D/T394C/V397C/D399K | 59 | 61 |
| | bond | | | |
| 121 | Steric hindrance | T350V/L351Y/T366L/K392L/T394W/F405A/ | 60 | 61 |
| | | Y407V | | |

### [Example 3] Evaluation of Physicochemical Properties of Antibodies with Amino Acid Modifications Introduced in the CH3 Interface

The effects of CH3 interface modifications on the expression level, monomer content (%), and thermal denaturation midpoint temperature (Tm) of antibody were compared. The yields of the antibodies prepared in Example 2 are shown in Table 3.

In addition, the monomer content in the purified antibodies was evaluated by the SEC analysis method described in Example 1. The results of the SEC analysis were analyzed to calculate the monomer content (%), where the components eluted on the higher molecular weight side than the monomer were collectively regarded as associated multimers, and the components eluted on the lower molecular weight side than the monomer were collectively regarded as degradation products. The analysis results are shown in Table 3.

The Tm of the antibodies subjected to amino acid modification was evaluated by differential scanning fluorimetry (DSF). It has already been reported that Tm determined by this method shows good correlation with Tm determined by differential scanning calorimetry, which is a widely-known method for evaluating the thermal stability of antibodies (Journal of Pharmaceutical Science 2010; 4:1707-1720).

A 5000×concentrate of SYPRO orange protein gel stain (Invitrogen) was diluted with PBS (Sigma), and an antibody solution was mixed with this detection dye. The 20-μL mixture was dispensed into a tube for measurement, and its temperature was raised from 30° C. to 99° C. at a rate of 240° C./hr using Rotor-Gene Q (QIAGEN). Fluorescent changes with increasing temperature were observed at 470 nm (excitation wavelength)/555 nm (fluorescence wavelength).

Obtained data was used to determine the temperature at which fluorescent transition was seen using Rotor-Gene Q Series Software (QIAGEN), and this value was determined as the Tm value. For the antibody used in this test, the Tm of Fab is about 95° C., which is markedly higher than the Tm of CH2 and CH3. Thus, in this test, the Tm value observed at the lowest temperature was regarded as a change in Tm resulting from Fc modification, and used for comparison. The results are shown in Table 3.

TABLE 3
Analysis results of antibodies with amino acid modifications

| | Full- | | | | | |
|---|---|---|---|---|---|---|
| | length | | | | | |
| | heavy | Light | Modifications that | SEC | | |
| | chain | chain | promote formation of | monomer | | |
| Sample | SEQ | SEQ | heavy chain | ratio | Tm | Yield |
| No. | ID NO | ID NO | homodimers | (%) | (° C.) | (mg) |
| 61 | 1 | 61 | — | 98.32 | 69 | 0.25 |
| 63 | 2 | 61 | E345K/K360E | 98.76 | 69 | 0.28 |
| 64 | 3 | 61 | E345R/K360E | 98.97 | 69.6 | 0.29 |
| 65 | 4 | 61 | E357K/K370E | 97.81 | 69.6 | 0.21 |
| 66 | 5 | 61 | D399K/K409E | 97.4 | 65.4 | 0.3 |
| 67 | 6 | 61 | D399K/K409D | 97.78 | 63 | 0.29 |
| 68 | 7 | 61 | D399R/K409E | 97.75 | 66 | 0.25 |
| 69 | 8 | 61 | D399R/K409D | 97.77 | 64.8 | 0.28 |
| 70 | 9 | 61 | K392E/D399K | 97.06 | 67.2 | 0.27 |
| 71 | 10 | 61 | K392D/D399K | 98.67 | 68.4 | 0.26 |
| 72 | 11 | 61 | K392E/D399R | 97.05 | 67.8 | 0.24 |
| 73 | 12 | 61 | K392D/D399R | 98.9 | 68.4 | 0.31 |
| 74 | 13 | 61 | K392C/D399C | 98.62 | 68.4 | 0.3 |
| 75 | 14 | 61 | Y349C/S354C | 97.98 | 69 | 0.26 |
| 76 | 15 | 61 | Y349C/E356C | 98.31 | 67.8 | 0.25 |
| 77 | 16 | 61 | Y349C/E357C | 98 | 69 | 0.25 |
| 78 | 17 | 61 | L351C/S354C | 92.42 | 68.4 | 0.26 |
| 79 | 18 | 61 | T394C/V397C | 84.93 | 68.4 | 0.15 |
| 80 | 19 | 61 | T394W/F405A | 97.65 | 68.4 | 0.22 |
| 81 | 20 | 61 | L351D/T366K | 93.99 | 63.6 | 0.23 |
| 82 | 21 | 61 | L351E/T366K | 76.86 | 58.8 | 0.31 |
| 83 | 22 | 61 | L351K/T366D | 97.02 | 67.2 | 0.25 |
| 84 | 23 | 61 | L351K/T366E | 86.6 | 60.6 | 0.31 |
| 85 | 24 | 61 | F405L/K409R | 95.26 | 66 | 0.27 |
| 86 | 25 | 61 | E356K/K439E | 98.74 | 69 | 0.29 |
| 87 | 26 | 61 | T366W/L368A/Y407V | 98.67 | 69 | 0.33 |
| 88 | 27 | 61 | T366Y/Y407T | 92.45 | 66.6 | 0.28 |
| 89 | 28 | 61 | T366W/Y407A | 90.2 | 69.6 | 0.25 |
| 90 | 29 | 61 | T366Y/T394W/F405A/Y407T | 85.25 | 61.2 | 0.23 |
| 91 | 30 | 61 | E357N/K370E/D399V/F405T/K409W | 91.82 | 63.6 | 0.27 |
| 92 | 31 | 61 | Q347R/K360E/D399V/F405T/K409W | 98.32 | 69 | 0.22 |
| 93 | 32 | 61 | Y349T/S364H/T394F/F405A | 68.34 | 53.4 | 0.15 |
| 94 | 33 | 61 | Y349T/S364H | 94.25 | 67.8 | 0.19 |
| 95 | 34 | 61 | T394F/F405A | 96.44 | 67.8 | 0.21 |
| 96 | 35 | 61 | E357K/K370E/D399K/K409E | 82.35 | 58.8 | 0.36 |
| 97 | 36 | 61 | E356K/D399K/K409E/K439E | 99.14 | 66 | 0.32 |
| 98 | 37 | 61 | E356K/E357K/K370E/K439E | 98.23 | 69 | 0.24 |
| 99 | 38 | 61 | E356K/E357K/K370E/D399K/K409E/K439E | 95.35 | 59.4 | 0.32 |
| 100 | 39 | 61 | Q347E/S354Y/T366Y/Y407T | 97.75 | 67.2 | 0.27 |
| 101 | 40 | 61 | T350V/L351Y/T366L/T394W/F405A | 85.08 | 63.6 | 0.32 |
| 102 | 41 | 61 | K392L/T394W/F405A/Y407V | 98.98 | 68.4 | 0.24 |
| 103 | 42 | 61 | Y349C/E357C/K392C/D399C | 54.85 | 54.6 | 0.07 |
| 104 | 43 | 61 | Y349C/L351C/S354C/E357C | 90.99 | 69 | 0.21 |
| 105 | 44 | 61 | Y349C/E357C/T394C/V397C | 90.51 | 68.4 | 0.06 |
| 106 | 45 | 61 | Y349C/E357C/K392C/T394C/V397C/D399C | 83.28 | 53.4 | 0.04 |
| 107 | 46 | 61 | K392C/T394C/V397C/D399C | 70.35 | 57.6 | 0.15 |
| 108 | 47 | 61 | L351C/S354C/K392C/D399C | 63.43 | 58.2 | 0.16 |
| 109 | 48 | 61 | E356K/K392D/D399K/K439E | 98.67 | 69.6 | 0.24 |
| 110 | 49 | 61 | E357K/K370E/K392D/D399K | 96.25 | 64.8 | 0.25 |
| 111 | 50 | 61 | K392D/D399K/K409D | 98.87 | 66 | 0.26 |
| 112 | 51 | 61 | Y349C/E357C/K392D/D399K | 97.2 | 68.4 | 0.05 |
| 113 | 52 | 61 | E357K/K370E/T394C/V397C | 98.22 | 67.2 | 0.22 |
| 114 | 53 | 61 | Y349C/E357C/K370E | 97.44 | 69.6 | 0.28 |
| 115 | 54 | 61 | E357K/K370E/K392C/D399C | 98.58 | 67.8 | 0.21 |
| 116 | 55 | 61 | E345R/Q347R/K360D/T366V/D399M/ | 98.36 | 52.8 | 0.27 |
| | | | Y407A/K409V | | | |
| 117 | 56 | 61 | E356K/K392D/D399K/K409D | 99.36 | 62.4 | 0.26 |
| 118 | 57 | 61 | E345R/Q347R/K360D/T366V | 90.8 | 66.6 | 0.26 |
| 119 | 58 | 61 | E345R/K360E/D399M/Y407A/K409V | 96.93 | 69.6 | 0.27 |
| 120 | 59 | 61 | K392D/T394C/V397C/D399K | 93.48 | 64.8 | 0.2 |
| 121 | 60 | 61 | T350V/L351Y/T366L/K392L/T394W/F405A/ | 97.23 | 69.6 | 0.24 |
| | | | Y407V | | | |

### [Example 4] Selection and Discussion of Modifications for Promoting Homodimerization of Heavy Chains

In the results of SEC analysis performed after expression of the full-length heavy chain, light chain, and Fc fragment as shown in Table 1, all modification pairs that exhibited a heterodimer formation ratio lower than that for WT-IgG1 (51.7%) can be said to provide a certain level of promotion of heavy-chain homodimer formation. As originally expected, the previously reported modifications for promoting the heteromeric association of heavy chains did not necessarily result in promotion of the homodimer formation of heavy chains. In particular, Sample Nos. 24, 33, and 57 resulted in considerable promotion of heavy chain heterodimerization, yielding no desired effect. It was also difficult for them to maintain the good physicochemical properties of antibody. Some modifications were found to greatly reduce the monomer ratio after SEC analysis, lower the Tm value, or reduce the antibody yield. This experimental fact suggests that modification pairs for promoting heavy-chain heterodimerization are not necessarily the same as those for promoting heavy-chain homodimerization, and it is important to discover pairs of amino acid modifications that promote homodimerization between heavy chains of one type and maintain good physicochemical properties. The present inventors evaluated the various antibodies shown in Table 1, and thereby discovered amino acid modifications that particularly potently suppress the heterodimerization of heavy chains and promote homodimerization of heavy chains. These selected antibodies are shown in Table 4. Compared to WT-IgG1, all these antibodies showed a comparable antibody expression level, a high monomer formation ratio, and a comparable Tm of the Fc region (however, a decrease was observed only in the antibody yield of Sample No. 112 compared to WT-IgG1).

TABLE 4
Amino acid modifications that exhibited strong
heavy-chain homodimerization-promoting ability

| Sample | Category of | Modifications that promote formation |
|---|---|---|
| No. | modification effect | of heavy chain homodimers |
| 80 | Steric hindrance | T394W/F405A |
| 87 | Steric hindrance | T366W/L368A/Y407V |
| 92 | Steric hindrance + | Q347R/K360E/D399V/F405T/ |
| | Charge | K409W |
| 95 | Steric hindrance | T394F/F405A |
| 97 | Charge | E356K/D399K/K409E/K439E |
| 100 | Steric hindrance | Q347E/S354Y/T366Y/Y407T |
| 102 | Steric hindrance | K392L/T394W/F405A/Y407V |
| 109 | Charge | E356K/K392D/D399K/K439E |
| 111 | Charge | K392D/D399K/K409D |
| 112 | Charge + Disulfide bond | Y349C/E357C/K392D/D399K |
| 116 | Steric hindrance + | E345R/Q347R/K360D/T366V/ |
| | Charge | D399M/Y407A/K409V |
| 117 | Charge | E356K/K392D/D399K/K409D |
| 119 | Steric hindrance + | E345R/K360E/D399M/Y407A/ |
| | Charge | K409V |
| 121 | Steric hindrance | T350V/L351Y/T366L/K392L/ |
| | | T394W/F405A/Y407V |

### [Example 5] Identification of Amino Acid Modification Pairs that Allow Coexpression of Multiple Antibodies

Next, in order to identify amino acid modification pairs that allow coexpression of multiple antibodies, genes were prepared such that the amino acid modifications shown in Table 4 were also introduced into the Fc fragment of IgG1. These exhaustive combinations were examined to find modification pairs that promote heavy-chain homodimer formation between modified Fc regions. For expression, the light chain of SEQ ID NO: 61 was used. Antibodies were expressed by the same method as described in Example 1. The homodimerization-promoting ability of each modification pair was determined based on whether the heterodimer level after SEC analysis was reduced as compared to a pair of identical modifications. (For example, when a pair of WT-IgG is expressed, the heterodimer level is 52%. Thus, a combination that shows a heterodimer level of less than 52% can be determined to be a pair having homodimerization-promoting ability. The SEQ ID NOs and combinations thereof used in the experiment, and their heterodimer formation ratios are shown in Table 5. Pairs of modifications in such a relation as to suppress heterodimerization in the experimental result are expected not to cause heteromeric association no matter how many of them are combined. Therefore, they can be used not only for coexpression of two antibodies but also for coexpression of three, four, five, six, seven, eight, nine, or more antibodies.

More specifically, amino acid modification pairs that showed a heteromultimer level of less than 10% in Table 5 can be used in combination. Table 6 shows effective amino acid modification pairs composed of these combinations (<10% Combinations 1-3512).

Furthermore, amino acid modification pairs composed only of pairs having stronger heavy-chain homomeric association-promoting ability (amino acid modification pairs that showed a heteromultimer level of 0% in Table 5) (0% Combinations 1-744) are shown in Table 7. In Tables 5, 6, and 7, the non-introduction of modifications is denoted as WT.

TABLE 5
Evaluation of heavy-chain homodimerization ability between selected modified antibodies

| | | | CH3 modification of Fc fragment | | | | | | | | | | | | | | |
|---|---|---|---|---|---|---|---|---|---|---|---|---|---|---|---|---|---|
| | | | | | | | | | | | | | | E345R/ | | | T350V/ |
| | | | | | | | | | | | | | | Q347R/ | | | L351Y/ |
| | | | | | | Q347R/ | | | | | | | | K360D/ | | E345R/ | T366L/ |
| | | | | | | K360E/ | | E356K/ | Q347E/ | K392L/ | E356K/ | | Y349C/ | T366V/ | E356K/ | K360E/ | K392L/ |
| | | | | | T366W/ | D399V/ | | D399K/ | S354Y/ | T394W/ | K392D/ | K392D/ | E357C/ | D399M/ | K392D/ | D399M/ | T394W/ |
| | | | | T394W/ | L368A/ | F405T/ | T394F/ | K409E/ | T366Y/ | F405A/ | D399K/ | D399K/ | K392D/ | Y407A/ | D399K/ | Y407A/ | F405A/ |
| | | | WT | F405A | Y407V | K409W | F405A | K439E | Y407T | Y407V | K439E | K409D | D399K | K409V | K409D | K409V | Y407V |
| | | | SEQ ID NO | | | | | | | | | | | | | | |
| | | | 63 | 64 | 65 | 66 | 67 | 68 | 69 | 70 | 71 | 72 | 73 | 74 | 75 | 76 | 77 |
| CH3 | WT | 1 | 52 | 0 | 0 | 0 | 0 | 0 | 0 | 0 | 0 | 0 | 0 | 0 | 0 | 0 | 0 |
| modification | T394W/F405A | 19 | 0 | 53 | 0 | 0 | 57 | 0 | 0 | 52 | 0 | 0 | 0 | 0 | 0 | 0 | 7 |
| of | T366W/L368A/ | 26 | 0 | 0 | 54 | 0 | 0 | 0 | 16 | 0 | 0 | 0 | 0 | 0 | 0 | 0 | 0 |
| full-length | Y407V | | | | | | | | | | | | | | | | |
| IgG | Q347R/K360E/ | 31 | 0 | 0 | 0 | 52 | 0 | 0 | 0 | 0 | 0 | 0 | 0 | 9 | 0 | 4 | 0 |
| | D399V/F405T/ | | | | | | | | | | | | | | | | |
| | K409W | | | | | | | | | | | | | | | | |
| | T394F/F405A | 34 | 0 | 59 | 0 | 0 | 53 | 0 | 0 | 47 | 0 | 0 | 0 | 0 | 0 | 0 | 1 |
| | E356K/D399K/ | 36 | 0 | 0 | 0 | 0 | 0 | 55 | 0 | 0 | 67 | 8 | 0 | 0 | 51 | 0 | 0 |
| | K409E/K439E | | | | | | | | | | | | | | | | |
| | Q347E/S354Y/ | 39 | 0 | 0 | 19 | 0 | 0 | 0 | 57 | 0 | 0 | 0 | 1 | 1 | 0 | 0 | 0 |
| | T366Y/Y407T | | | | | | | | | | | | | | | | |
| | K392L/T394W/ | 41 | 0 | 55 | 0 | 0 | 47 | 0 | 0 | 53 | 0 | 0 | 0 | 0 | 0 | 0 | 66 |
| | F405A/Y407V | | | | | | | | | | | | | | | | |
| | E356K/K392D/ | 48 | 0 | 0 | 0 | 0 | 0 | 63 | 0 | 0 | 53 | 7 | 0 | 0 | 55 | 0 | 0 |
| | D399K/K439E | | | | | | | | | | | | | | | | |
| | K392D/D399K/ | 50 | 0 | 0 | 0 | 0 | 0 | 7 | 0 | 0 | 6 | 54 | 0 | 0 | 47 | 4 | 0 |
| | K409D | | | | | | | | | | | | | | | | |
| | Y349C/E357C/ | 51 | 0 | 0 | 0 | 0 | 0 | 0 | 0 | 0 | 0 | 9 | 56 | 2 | 0 | 0 | 0 |
| | K392D/D399K | | | | | | | | | | | | | | | | |
| | E345R/Q347R/ | 55 | 0 | 0 | 0 | 6 | 0 | 0 | 0 | 0 | 0 | 0 | 0 | 52 | 0 | 49 | 0 |
| | K360D/T366V/ | | | | | | | | | | | | | | | | |
| | D399M/Y407A/ | | | | | | | | | | | | | | | | |
| | K409V | | | | | | | | | | | | | | | | |
| | E356K/K392D/ | 56 | 0 | 0 | 0 | 0 | 0 | 53 | 0 | 0 | 60 | 51 | 0 | 0 | 56 | 0 | 0 |
| | D399K/K409D | | | | | | | | | | | | | | | | |
| | E345R/K360E/ | 58 | 0 | 0 | 0 | 4 | 0 | 0 | 0 | 0 | 0 | 3 | 0 | 51 | 0 | 51 | 0 |
| | D399M/Y407A/ | | | | | | | | | | | | | | | | |
| | K409V | | | | | | | | | | | | | | | | |
| | T350V/L351Y/ | 60 | 0 | 6 | 0 | 0 | 4 | 0 | 0 | 66 | 0 | 0 | 0 | 0 | 0 | 0 | 51 |
| | T366L/K392L/ | | | | | | | | | | | | | | | | |
| | T394W/F405A/ | | | | | | | | | | | | | | | | |
| | Y407V | | | | | | | | | | | | | | | | |

Lengthy table referenced here
US20260217835A1-20260730-T00001
Please refer to the end of the specification for access instructions.

TABLE 7
Amino acid modification pairs that can be used for coexpression of two to six antibodies (composed
of amino acid modification pairs that showed a heteromultimer level of 0% in Table 5)

| Amino acid | | | | | | |
|---|---|---|---|---|---|---|
| modification pairs | Antibody 1 | Antibody 2 | Antibody 3 | Antibody 4 | Antibody 5 | Antibody 6 |
| 0% Combi | WT | T394W/F405A | | | | |
| nation 1 | | | | | | |
| 0% Combi | WT | T366W/L368A/ | | | | |
| nation 2 | | Y407V | | | | |
| 0% Combi | WT | Q347R/K360E/ | | | | |
| nation 3 | | D399V/F405T/ | | | | |
| | | K409W | | | | |
| 0% Combi | WT | E356K/D399K/ | | | | |
| nation 4 | | K409E/K439E | | | | |
| 0% Combi | WT | Y349C/E357C/ | | | | |
| nation 5 | | K392D/D399K | | | | |
| 0% Combi | WT | E345R/Q347R/ | | | | |
| nation 6 | | K360D/T366V/ | | | | |
| | | D399M/Y407A/ | | | | |
| | | K409V | | | | |
| 0% Combi | WT | Q347E/S354Y/ | | | | |
| nation 7 | | T366Y/Y407T | | | | |
| 0% Combi | WT | E356K/K392D/ | | | | |
| nation 8 | | D399K/K439E | | | | |
| 0% Combi | WT | K392D/D399K/ | | | | |
| nation 9 | | K409D | | | | |
| 0% Combi | WT | E356K/K392D/ | | | | |
| nation 10 | | D399K/K409D | | | | |
| 0% Combi | WT | E345R/K360E/ | | | | |
| nation 11 | | D399M/Y407A/ | | | | |
| | | K409V | | | | |
| 0% Combi | WT | T394F/F405A | | | | |
| nation 12 | | | | | | |
| 0% Combi | WT | K392L/T394W/ | | | | |
| nation 13 | | F405A/Y407V | | | | |
| 0% Combi | WT | T350V/L351Y/ | | | | |
| nation 14 | | T366L/K392L/ | | | | |
| | | T394W/F405A/ | | | | |
| | | Y407V | | | | |
| 0% Combi | T394W/F405A | T366W/L368A/ | | | | |
| nation 15 | | Y407V | | | | |
| 0% Combi | T394W/F405A | Q347R/K360E/ | | | | |
| nation 16 | | D399V/F405T/ | | | | |
| | | K409W | | | | |
| 0% Combi | T394W/F405A | E356K/D399K/ | | | | |
| nation 17 | | K409E/K439E | | | | |
| 0% Combi | T394W/F405A | Y349C/E357C/ | | | | |
| nation 18 | | K392D/D399K | | | | |
| 0% Combi | T394W/F405A | E345R/Q347R/ | | | | |
| nation 19 | | K360D/T366V/ | | | | |
| | | D399M/Y407A/ | | | | |
| | | K409V | | | | |
| 0% Combi | T394W/F405A | Q347E/S354Y/ | | | | |
| nation 20 | | T366Y/Y407T | | | | |
| 0% Combi | T394W/F405A | E356K/K392D/ | | | | |
| nation 21 | | D399K/K439E | | | | |
| 0% Combi | T394W/F405A | K392D/D399K/ | | | | |
| nation 22 | | K409D | | | | |
| 0% Combi | T394W/F405A | E356K/K392D/ | | | | |
| nation 23 | | D399K/K409D | | | | |
| 0% Combi | T394W/F405A | E345R/K360E/ | | | | |
| nation 24 | | D399M/Y407A/ | | | | |
| | | K409V | | | | |
| 0% Combi | T366W/L368A/ | Q347R/K360E/ | | | | |
| nation 25 | Y407V | D399V/F405T/ | | | | |
| | | K409W | | | | |
| 0% Combi | T366W/L368A/ | E356K/D399K/ | | | | |
| nation 26 | Y407V | K409E/K439E | | | | |
| 0% Combi | T366W/L368A/ | Y349C/E357C/ | | | | |
| nation 27 | Y407V | K392D/D399K | | | | |
| 0% Combi | T366W/L368A/ | E345R/Q347R/ | | | | |
| nation 28 | Y407V | K360D/T366V/ | | | | |
| | | D399M/Y407A/ | | | | |
| | | K409V | | | | |
| 0% Combi | T366W/L368A/ | E356K/K392D/ | | | | |
| nation 29 | Y407V | D399K/K439E | | | | |
| 0% Combi | T366W/L368A/ | K392D/D399K/ | | | | |
| nation 30 | Y407V | K409D | | | | |
| 0% Combi | T366W/L368A/ | E356K/K392D/ | | | | |
| nation 31 | Y407V | D399K/K409D | | | | |
| 0% Combi | T366W/L368A/ | E345R/K360E/ | | | | |
| nation 32 | Y407V | D399M/Y407A/ | | | | |
| | | K409V | | | | |
| 0% Combi | T366W/L368A/ | T394F/F405A | | | | |
| nation 33 | Y407V | | | | | |
| 0% Combi | T366W/L368A/ | K392L/T394W/ | | | | |
| nation 34 | Y407V | F405A/Y407V | | | | |
| 0% Combi | T366W/L368A/ | T350V/L351Y/ | | | | |
| nation 35 | Y407V | T366L/K392L/ | | | | |
| | | T394W/F405A/ | | | | |
| | | Y407V | | | | |
| 0% Combi | Q347R/K360E/ | E356K/D399K/ | | | | |
| nation 36 | D399V/F405T/ | K409E/K439E | | | | |
| | K409W | | | | | |
| 0% Combi | Q347R/K360E/ | Y349C/E357C/ | | | | |
| nation 37 | D399V/F405T/ | K392D/D399K | | | | |
| | K409W | | | | | |
| 0% Combi | Q347R/K360E/ | Q347E/S354Y/ | | | | |
| nation 38 | D399V/F405T/ | T366Y/Y407T | | | | |
| | K409W | | | | | |
| 0% Combi | Q347R/K360E/ | E356K/K392D/ | | | | |
| nation 39 | D399V/F405T/ | D399K/K439E | | | | |
| | K409W | | | | | |
| 0% Combi | Q347R/K360E/ | K392D/D399K/ | | | | |
| nation 40 | D399V/F405T/ | K409D | | | | |
| | K409W | | | | | |
| 0% Combi | Q347R/K360E/ | E356K/K392D/ | | | | |
| nation 41 | D399V/F405T/ | D399K/K409D | | | | |
| | K409W | | | | | |
| 0% Combi | Q347R/K360E/ | T394F/F405A | | | | |
| nation 42 | D399V/F405T/ | | | | | |
| | K409W | | | | | |
| 0% Combi | Q347R/K360E/ | K392L/T394W/ | | | | |
| nation 43 | D399V/F405T/ | F405A/Y407V | | | | |
| | K409W | | | | | |
| 0% Combi | Q347R/K360E/ | T350V/L351Y/ | | | | |
| nation 44 | D399V/F405T/ | T366L/K392L/ | | | | |
| | K409W | T394W/F405A/ | | | | |
| | | Y407V | | | | |
| 0% Combi | E356K/D399K/ | Y349C/E357C/ | | | | |
| nation 45 | K409E/K439E | K392D/D399K | | | | |
| 0% Combi | E356K/D399K/ | E345R/Q347R/ | | | | |
| nation 46 | K409E/K439E | K360D/T366V/ | | | | |
| | | D399M/Y407A/ | | | | |
| | | K409V | | | | |
| 0% Combi | E356K/D399K/ | Q347E/S354Y/ | | | | |
| nation 47 | K409E/K439E | T366Y/Y407T | | | | |
| 0% Combi | E356K/D399K/ | E345R/K360E/ | | | | |
| nation 48 | K409E/K439E | D399M/Y407A/ | | | | |
| | | K409V | | | | |
| 0% Combi | E356K/D399K/ | T394F/F405A | | | | |
| nation 49 | K409E/K439E | | | | | |
| 0% Combi | E356K/D399K/ | K392L/T394W/ | | | | |
| nation 50 | K409E/K439E | F405A/Y407V | | | | |
| 0% Combi | E356K/D399K/ | T350V/L351Y/ | | | | |
| nation 51 | K409E/K439E | T366L/K392L/ | | | | |
| | | T394W/F405A/ | | | | |
| | | Y407V | | | | |
| 0% Combi | Y349C/E357C/ | E356K/K392D/ | | | | |
| nation 52 | K392D/D399K | D399K/K439E | | | | |
| 0% Combi | Y349C/E357C/ | E356K/K392D/ | | | | |
| nation 53 | K392D/D399K | D399K/K409D | | | | |
| 0% Combi | Y349C/E357C/ | E345R/K360E/ | | | | |
| nation 54 | K392D/D399K | D399M/Y407A/ | | | | |
| | | K409V | | | | |
| 0% Combi | Y349C/E357C/ | T394F/F405A | | | | |
| nation 55 | K392D/D399K | | | | | |

| | | | | | |
|---|---|---|---|---|---|
| | | | | | Y407A/K409V |
| <10% Combi | WT | T366W/L368A/ | Q347R/K360E/ | Y349C/E357C/ | E356K/K392D/ |
| nation 3434 | | Y407V | D399V/F405T/ | K392D/D399K | D399K/K439E |
| | | | K409W | | |
| <10% Combi | WT | T366W/L368A/ | Q347R/K360E/ | Y349C/E357C/ | E356K/K392D/ |
| nation 3435 | | Y407V | D399V/F405T/ | K392D/D399K | D399K/K439E |
| | | | K409W | | |
| <10% Combi | WT | T366W/L368A/ | Q347R/K360E/ | Y349C/E357C/ | E356K/K392D/ |
| nation 3436 | | Y407V | D399V/F405T/ | K392D/D399K | D399K/K439E |
| | | | K409W | | |
| <10% Combi | WT | T366W/L368A/ | Q347R/K360E/ | Y349C/E357C/ | E356K/K392D/ |
| nation 3437 | | Y407V | D399V/F405T/ | K392D/D399K | D399K/K439E |
| | | | K409W | | |
| <10% Combi | WT | T366W/L368A/ | Q347R/K360E/ | Y349C/E357C/ | E356K/K392D/ |
| nation 3438 | | Y407V | D399V/F405T/ | K392D/D399K | D399K/K439E |
| | | | K409W | | |
| <10% Combi | WT | T366W/L368A/ | Q347R/K360E/ | Y349C/E357C/ | K392D/D399K/ |
| nation 3439 | | Y407V | D399V/F405T/ | K392D/D399K | K409D |
| | | | K409W | | |
| <10% Combi | WT | T366W/L368A/ | Q347R/K360E/ | Y349C/E357C/ | E356K/K392D/ |
| nation 3440 | | Y407V | D399V/F405T/ | K392D/D399K | D399K/K409D |
| | | | K409W | | |
| <10% Combi | WT | T366W/L368A/ | Q347R/K360E/ | E345R/Q347R/ | E356K/K392D/ |
| nation 3441 | | Y407V | D399V/F405T/ | K360D/T366V/ | D399K/K439E |
| | | | K409W | D399M/Y407A/ | |
| | | | | K409V | |
| <10% Combi | WT | T366W/L368A/ | Q347R/K360E/ | E356K/K392D/ | K392D/D399K/ |
| nation 3442 | | Y407V | D399V/F405T/ | D399K/K439E | K409D |
| | | | K409W | | |
| <10% Combi | WT | T366W/L368A/ | E356K/D399K/ | Y349C/E357C/ | E345R/Q347R/ |
| nation 3443 | | Y407V | K409E/K439E | K392D/D399K | K360D/T366V/ |
| | | | | | D399M/Y407A/ |
| | | | | | K409V |
| <10% Combi | WT | T366W/L368A/ | E356K/D399K/ | Y349C/E357C/ | K392D/D399K/ |
| nation 3444 | | Y407V | K409E/K439E | K392D/D399K | K409D |
| <10% Combi | WT | T366W/L368A/ | Y349C/E357C/ | E345R/Q347R/ | E356K/K392D/ |
| nation 3445 | | Y407V | K392D/D399K | K360D/T366V/ | D399K/K439E |
| | | | | D399M/Y407A/ | |
| | | | | K409V | |
| <10% Combi | WT | T366W/L368A/ | Y349C/E357C/ | E356K/K392D/ | K392D/D399K/ |
| nation 3446 | | Y407V | K392D/D399K | D399K/K439E | K409D |
| <10% Combi | WT | Q347R/K360E/ | E356K/D399K/ | Y349C/E357C/ | E345R/Q347R/ |
| nation 3447 | | D399V/F405T/ | K409E/K439E | K392D/D399K | K360D/T366V/ |
| | | K409W | | | D399M/Y407A/ |
| | | | | | K409V |
| <10% Combi | WT | Q347R/K360E/ | E356K/D399K/ | Y349C/E357C/ | E345R/Q347R/ |
| nation 3448 | | D399V/F405T/ | K409E/K439E | K392D/D399K | K360D/T366V/ |
| | | K409W | | | D399M/Y407A/ |
| | | | | | K409V |
| <10% Combi | WT | Q347R/K360E/ | E356K/D399K/ | Y349C/E357C/ | E345R/Q347R/ |
| nation 3449 | | D399V/F405T/ | K409E/K439E | K392D/D399K | K360D/T366V/ |
| | | K409W | | | D399M/Y407A/ |
| | | | | | K409V |
| <10% Combi | WT | Q347R/K360E/ | E356K/D399K/ | Y349C/E357C/ | E345R/Q347R/ |
| nation 3450 | | D399V/F405T/ | K409E/K439E | K392D/D399K | K360D/T366V/ |
| | | K409W | | | D399M/Y407A/ |
| | | | | | K409V |
| <10% Combi | WT | Q347R/K360E/ | E356K/D399K/ | Y349C/E357C/ | E345R/Q347R/ |
| nation 3451 | | D399V/F405T/ | K409E/K439E | K392D/D399K | K360D/T366V/ |
| | | K409W | | | D399M/Y407A/ |
| | | | | | K409V |
| <10% Combi | WT | Q347R/K360E/ | E356K/D399K/ | Y349C/E357C/ | Q347E/S354Y/ |
| nation 3452 | | D399V/F405T/ | K409E/K439E | K392D/D399K | T366Y/Y407T |
| | | K409W | | | |
| <10% Combi | WT | Q347R/K360E/ | E356K/D399K/ | Y349C/E357C/ | Q347E/S354Y/ |
| nation 3453 | | D399V/F405T/ | K409E/K439E | K392D/D399K | T366Y/Y407T |
| | | K409W | | | |
| <10% Combi | WT | Q347R/K360E/ | E356K/D399K/ | Y349C/E357C/ | Q347E/S354Y/ |
| nation 3454 | | D399V/F405T/ | K409E/K439E | K392D/D399K | T366Y/Y407T |
| | | K409W | | | |
| <10% Combi | WT | Q347R/K360E/ | E356K/D399K/ | Y349C/E357C/ | Q347E/S354Y/ |
| nation 3455 | | D399V/F405T/ | K409E/K439E | K392D/D399K | T366Y/Y407T |
| | | K409W | | | |
| <10% Combi | WT | Q347R/K360E/ | E356K/D399K/ | Y349C/E357C/ | Q347E/S354Y/ |
| nation 3456 | | D399V/F405T/ | K409E/K439E | K392D/D399K | T366Y/Y407T |
| | | K409W | | | |
| <10% Combi | WT | Q347R/K360E/ | E356K/D399K/ | Y349C/E357C/ | K392D/D399K/ |
| nation 3457 | | D399V/F405T/ | K409E/K439E | K392D/D399K | K409D |
| | | K409W | | | |
| <10% Combi | WT | Q347R/K360E/ | E356K/D399K/ | E345R/Q347R/ | Q347E/S354Y/ |
| nation 3458 | | D399V/F405T/ | K409E/K439E | K360D/T366V/ | T366Y/Y407T |
| | | K409W | | D399M/Y407A/ | |
| | | | | K409V | |
| <10% Combi | WT | Q347R/K360E/ | E356K/D399K/ | Q347E/S354Y/ | K392D/D399K/ |
| nation 3459 | | D399V/F405T/ | K409E/K439E | T366Y/Y407T | K409D |
| | | K409W | | | |
| <10% Combi | WT | Q347R/K360E/ | Y349C/E357C/ | E345R/Q347R/ | Q347E/S354Y/ |
| nation 3460 | | D399V/F405T/ | K392D/D399K | K360D/T366V/ | T366Y/Y407T |
| | | K409W | | D399M/Y407A/ | |
| | | | | K409V | |
| <10% Combi | WT | Q347R/K360E/ | Y349C/E357C/ | E345R/Q347R/ | Q347E/S354Y/ |
| nation 3461 | | D399V/F405T/ | K392D/D399K | K360D/T366V/ | T366Y/Y407T |
| | | K409W | | D399M/Y407A/ | |
| | | | | K409V | |
| <10% Combi | WT | Q347R/K360E/ | Y349C/E357C/ | E345R/Q347R/ | Q347E/S354Y/ |
| nation 3462 | | D399V/F405T/ | K392D/D399K | K360D/T366V/ | T366Y/Y407T |
| | | K409W | | D399M/Y407A/ | |
| | | | | K409V | |
| <10% Combi | WT | Q347R/K360E/ | Y349C/E357C/ | E345R/Q347R/ | Q347E/S354Y/ |
| nation 3463 | | D399V/F405T/ | K392D/D399K | K360D/T366V/ | T366Y/Y407T |
| | | K409W | | D399M/Y407A/ | |
| | | | | K409V | |
| <10% Combi | WT | Q347R/K360E/ | Y349C/E357C/ | E345R/Q347R/ | Q347E/S354Y/ |
| nation 3464 | | D399V/F405T/ | K392D/D399K | K360D/T366V/ | T366Y/Y407T |
| | | K409W | | D399M/Y407A/ | |
| | | | | K409V | |
| <10% Combi | WT | Q347R/K360E/ | Y349C/E357C/ | E345R/Q347R/ | Q347E/S354Y/ |
| nation 3465 | | D399V/F405T/ | K392D/D399K | K360D/T366V/ | T366Y/Y407T |
| | | K409W | | D399M/Y407A/ | |
| | | | | K409V | |
| <10% Combi | WT | Q347R/K360E/ | Y349C/E357C/ | E345R/Q347R/ | E356K/K392D/ |
| nation 3466 | | D399V/F405T/ | K392D/D399K | K360D/T366V/ | D399K/K439E |
| | | K409W | | D399M/Y407A/ | |
| | | | | K409V | |
| <10% Combi | WT | Q347R/K360E/ | Y349C/E357C/ | Q347E/S354Y/ | E356K/K392D/ |
| nation 3467 | | D399V/F405T/ | K392D/D399K | T366Y/Y407T | D399K/K439E |
| | | K409W | | | |
| <10% Combi | WT | Q347R/K360E/ | Y349C/E357C/ | Q347E/S354Y/ | E356K/K392D/ |
| nation 3468 | | D399V/F405T/ | K392D/D399K | T366Y/Y407T | D399K/K439E |
| | | K409W | | | |
| <10% Combi | WT | Q347R/K360E/ | Y349C/E357C/ | Q347E/S354Y/ | E356K/K392D/ |
| nation 3469 | | D399V/F405T/ | K392D/D399K | T366Y/Y407T | D399K/K439E |
| | | K409W | | | |
| <10% Combi | WT | Q347R/K360E/ | Y349C/E357C/ | Q347E/S354Y/ | E356K/K392D/ |
| nation 3470 | | D399V/F405T/ | K392D/D399K | T366Y/Y407T | D399K/K439E |
| | | K409W | | | |
| <10% Combi | WT | Q347R/K360E/ | Y349C/E357C/ | Q347E/S354Y/ | E356K/K392D/ |
| nation 3471 | | D399V/F405T/ | K392D/D399K | T366Y/Y407T | D399K/K439E |
| | | K409W | | | |
| <10% Combi | WT | Q347R/K360E/ | Y349C/E357C/ | Q347E/S354Y/ | K392D/D399K/ |
| nation 3472 | | D399V/F405T/ | K392D/D399K | T366Y/Y407T | K409D |
| | | K409W | | | |
| <10% Combi | WT | Q347R/K360E/ | Y349C/E357C/ | Q347E/S354Y/ | E356K/K392D/ |
| nation 3473 | | D399V/F405T/ | K392D/D399K | T366Y/Y407T | D399K/K409D |
| | | K409W | | | |
| <10% Combi | WT | Q347R/K360E/ | Y349C/E357C/ | E356K/K392D/ | K392D/D399K/ |
| nation 3474 | | D399V/F405T/ | K392D/D399K | D399K/K439E | K409D |
| | | K409W | | | |
| <10% Combi | WT | Q347R/K360E/ | E345R/Q347R/ | Q347E/S354Y/ | E356K/K392D/ |
| nation 3475 | | D399V/F405T/ | K360D/T366V/ | T366Y/Y407T | D399K/K439E |
| | | K409W | D399M/Y407A/ | | |
| | | | K409V | | |
| <10% Combi | WT | Q347R/K360E/ | Q347E/S354Y/ | E356K/K392D/ | K392D/D399K/ |
| nation 3476 | | D399V/F405T/ | T366Y/Y407T | D399K/K439E | K409D |
| | | K409W | | | |
| <10% Combi | WT | E356K/D399K/ | Y349C/E357C/ | E345R/Q347R/ | Q347E/S354Y/ |
| nation 3477 | | K409E/K439E | K392D/D399K | K360D/T366V/ | T366Y/Y407T |
| | | | | D399M/Y407A/ | |
| | | | | K409V | |
| <10% Combi | WT | E356K/D399K/ | Y349C/E357C/ | Q347E/S354Y/ | K392D/D399K/ |
| nation 3478 | | K409E/K439E | K392D/D399K | T366Y/Y407T | K409D |
| <10% Combi | WT | Y349C/E357C/ | E345R/Q347R/ | Q347E/S354Y/ | E356K/K392D/ |
| nation 3479 | | K392D/D399K | K360D/T366V/ | T366Y/Y407T | D399K/K439E |
| | | | D399M/Y407A/ | | |
| | | | K409V | | |
| <10% Combi | WT | Y349C/E357C/ | Q347E/S354Y/ | E356K/K392D/ | K392D/D399K/ |
| nation 3480 | | K392D/D399K | T366Y/Y407T | D399K/K439E | K409D |
| <10% Combi | T394W/F405A | T366W/L368A/ | Q347R/K360E/ | E356K/D399K/ | Y349C/E357C/ |
| nation 3481 | | Y407V | D399V/F405T/ | K409E/K439E | K392D/D399K |
| | | | K409W | | |
| <10% Combi | T394W/F405A | T366W/L368A/ | Q347R/K360E/ | E356K/D399K/ | Y349C/E357C/ |
| nation 3482 | | Y407V | D399V/F405T/ | K409E/K439E | K392D/D399K |
| | | | K409W | | |
| <10% Combi | T394W/F405A | T366W/L368A/ | Q347R/K360E/ | Y349C/E357C/ | E345R/Q347R/ |
| nation 3483 | | Y407V | D399V/F405T/ | K392D/D399K | K360D/T366V/ |
| | | | K409W | | D399M/Y407A/ |
| | | | | | K409V |
| <10% Combi | T394W/F405A | T366W/L368A/ | Q347R/K360E/ | Y349C/E357C/ | E356K/K392D/ |
| nation 3484 | | Y407V | D399V/F405T/ | K392D/D399K | D399K/K439E |
| | | | K409W | | |
| <10% Combi | T394W/F405A | Q347R/K360E/ | E356K/D399K/ | Y349C/E357C/ | E345R/Q347R/ |
| nation 3485 | | D399V/F405T/ | K409E/K439E | K392D/D399K | K360D/T366V/ |
| | | K409W | | | D399M/Y407A/ |
| | | | | | K409V |
| <10% Combi | T394W/F405A | Q347R/K360E/ | E356K/D399K/ | Y349C/E357C/ | Q347E/S354Y/ |
| nation 3486 | | D399V/F405T/ | K409E/K439E | K392D/D399K | T366Y/Y407T |
| | | K409W | | | |
| <10% Combi | T394W/F405A | Q347R/K360E/ | Y349C/E357C/ | E345R/Q347R/ | Q347E/S354Y/ |
| nation 3487 | | D399V/F405T/ | K392D/D399K | K360D/T366V/ | T366Y/Y407T |
| | | K409W | | D399M/Y407A/ | |
| | | | | K409V | |
| <10% Combi | T394W/F405A | Q347R/K360E/ | Y349C/E357C/ | Q347E/S354Y/ | E356K/K392D/ |
| nation 3488 | | D399V/F405T/ | K392D/D399K | T366Y/Y407T | D399K/K439E |
| | | K409W | | | |
| <10% Combi | T366W/L368A/ | Q347R/K360E/ | E356K/D399K/ | Y349C/E357C/ | E345R/Q347R/ |
| nation 3489 | Y407V | D399V/F405T/ | K409E/K439E | K392D/D399K | K360D/T366V/ |
| | | K409W | | | D399M/Y407A/ |
| | | | | | K409V |
| <10% Combi | T366W/L368A/ | Q347R/K360E/ | E356K/D399K/ | Y349C/E357C/ | K392D/D399K/ |
| nation 3490 | Y407V | D399V/F405T/ | K409E/K439E | K392D/D399K | K409D |
| | | K409W | | | |
| <10% Combi | T366W/L368A/ | Q347R/K360E/ | Y349C/E357C/ | E345R/Q347R/ | E356K/K392D/ |
| nation 3491 | Y407V | D399V/F405T/ | K392D/D399K | K360D/T366V/ | D399K/K439E |
| | | K409W | | D399M/Y407A/ | |
| | | | | K409V | |
| <10% Combi | T366W/L368A/ | Q347R/K360E/ | Y349C/E357C/ | E356K/K392D/ | K392D/D399K/ |
| nation 3492 | Y407V | D399V/F405T/ | K392D/D399K | D399K/K439E | K409D |
| | | K409W | | | |
| <10% Combi | Q347R/K360E/ | E356K/D399K/ | Y349C/E357C/ | E345R/Q347R/ | Q347E/S354Y/ |
| nation 3493 | D399V/F405T/ | K409E/K439E | K392D/D399K | K360D/T366V/ | T366Y/Y407T |
| | K409W | | | D399M/Y407A/ | |
| | | | | K409V | |
| <10% Combi | Q347R/K360E/ | E356K/D399K/ | Y349C/E357C/ | Q347E/S354Y/ | K392D/D399K/ |
| nation 3494 | D399V/F405T/ | K409E/K439E | K392D/D399K | T366Y/Y407T | K409D |
| | K409W | | | | |
| <10% Combi | Q347R/K360E/ | Y349C/E357C/ | E345R/Q347R/ | Q347E/S354Y/ | E356K/K392D/ |
| nation 3495 | D399V/F405T/ | K392D/D399K | K360D/T366V/ | T366Y/Y407T | D399K/K439E |
| | K409W | | D399M/Y407A/ | | |
| | | | K409V | | |
| <10% Combi | Q347R/K360E/ | Y349C/E357C/ | Q347E/S354Y/ | E356K/K392D/ | K392D/D399K/ |
| nation 3496 | D399V/F405T/ | K392D/D399K | T366Y/Y407T | D399K/K439E | K409D |
| | K409W | | | | |
| <10% Combi | WT | T394W/F405A | T366W/L368A/ | Q347R/K360E/ | E356K/D399K/ |
| nation 3497 | | | Y407V | D399V/F405T/ | K409E/K439E |
| | | | | K409W | |
| <10% Combi | WT | T394W/F405A | T366W/L368A/ | Q347R/K360E/ | E356K/D399K/ |
| nation 3498 | | | Y407V | D399V/F405T/ | K409E/K439E |
| | | | | K409W | |
| <10% Combi | WT | T394W/F405A | T366W/L368A/ | Q347R/K360E/ | Y349C/E357C/ |
| nation 3499 | | | Y407V | D399V/F405T/ | K392D/D399K |
| | | | | K409W | |
| <10% Combi | WT | T394W/F405A | T366W/L368A/ | Q347R/K360E/ | Y349C/E357C/ |
| nation 3500 | | | Y407V | D399V/F405T/ | K392D/D399K |
| | | | | K409W | |
| <10% Combi | WT | T394W/F405A | Q347R/K360E/ | E356K/D399K/ | Y349C/E357C/ |
| nation 3501 | | | D399V/F405T/ | K409E/K439E | K392D/D399K |
| | | | K409W | | |
| <10% Combi | WT | T394W/F405A | Q347R/K360E/ | E356K/D399K/ | Y349C/E357C/ |
| nation 3502 | | | D399V/F405T/ | K409E/K439E | K392D/D399K |
| | | | K409W | | |
| <10% Combi | WT | T394W/F405A | Q347R/K360E/ | Y349C/E357C/ | E345R/Q347R/ |
| nation 3503 | | | D399V/F405T/ | K392D/D399K | K360D/T366V/ |
| | | | K409W | | D399M/Y407A/ |
| | | | | | K409V |
| <10% Combi | WT | T394W/F405A | Q347R/K360E/ | Y349C/E357C/ | Q347E/S354Y/ |
| nation 3504 | | | D399V/F405T/ | K392D/D399K | T366Y/Y407T |
| | | | K409W | | |
| <10% Combi | WT | T366W/L368A/ | Q347R/K360E/ | E356K/D399K/ | Y349C/E357C/ |
| nation 3505 | | Y407V | D399V/F405T/ | K409E/K439E | K392D/D399K |
| | | | K409W | | |
| <10% Combi | WT | T366W/L368A/ | Q347R/K360E/ | E356K/D399K/ | Y349C/E357C/ |
| nation 3506 | | Y407V | D399V/F405T/ | K409E/K439E | K392D/D399K |
| | | | K409W | | |
| <10% Combi | WT | T366W/L368A/ | Q347R/K360E/ | Y349C/E357C/ | E345R/Q347R/ |
| nation 3507 | | Y407V | D399V/F405T/ | K392D/D399K | K360D/T366V/ |
| | | | K409W | | D399M/Y407A/ |
| | | | | | K409V |
| <10% Combi | WT | T366W/L368A/ | Q347R/K360E/ | Y349C/E357C/ | E356K/K392D/ |
| nation 3508 | | Y407V | D399V/F405T/ | K392D/D399K | D399K/K439E |
| | | | K409W | | |
| <10% Combi | WT | Q347R/K360E/ | E356K/D399K/ | Y349C/E357C/ | E345R/Q347R/ |
| nation 3509 | | D399V/F405T/ | K409E/K439E | K392D/D399K | K360D/T366V/ |
| | | K409W | | | D399M/Y407A/ |
| | | | | | K409V |
| <10% Combi | WT | Q347R/K360E/ | E356K/D399K/ | Y349C/E357C/ | Q347E/S354Y/ |
| nation 3510 | | D399V/F405T/ | K409E/K439E | K392D/D399K | T366Y/Y407T |
| | | K409W | | | |
| <10% Combi | WT | Q347R/K360E/ | Y349C/E357C/ | E345R/Q347R/ | Q347E/S354Y/ |
| nation 3511 | | D399V/F405T/ | K392D/D399K | K360D/T366V/ | T366Y/Y407T |
| | | K409W | | D399M/Y407A/ | |
| | | | | K409V | |
| <10% Combi | WT | Q347R/K360E/ | Y349C/E357C/ | Q347E/S354Y/ | E356K/K392D/ |
| nation 3512 | | D399V/F405T/ | K392D/D399K | T366Y/Y407T | D399K/K439E |
| | | K409W | | | |

| | | | | |
|---|---|---|---|---|
| | | | Y405A/K407V | |
| <10% Combi | K392D/D399K/ | E345R/K360E/ | T394F/F405A | |
| nation 3434 | K409D | D399M/Y407A/ | | |
| | | K409V | | |
| <10% Combi | K392D/D399K/ | E345R/K360E/ | K392L/T394W/ | |
| nation 3435 | K409D | D399M/Y407A/ | F405A/Y407V | |
| | | K409V | | |
| <10% Combi | K392D/D399K/ | E345R/K360E/ | T350V/L351Y/ | |
| nation 3436 | K409D | D399M/Y407A/ | T366L/K392L/ | |
| | | K409V | T394W/F405A/ | |
| | | | Y407V | |
| <10% Combi | K392D/D399K/ | T394F/F405A | T350V/L351Y/ | |
| nation 3437 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3438 | D399M/Y407A/ | | T366L/K392L/ | |
| | K409V | | T394W/F405A/ | |
| | | | Y407V | |
| <10% Combi | E345R/K360E/ | T394F/F405A | T350V/L351Y/ | |
| nation 3439 | D399M/Y407A/ | | T366L/K392L/ | |
| | K409V | | T394W/F405A/ | |
| | | | Y407V | |
| <10% Combi | E345R/K360E/ | T394F/F405A | T350V/L351Y/ | |
| nation 3440 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3441 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3442 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3443 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3444 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3445 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3446 | D399M/Y407A/ | | T366L/K392L/ | |
| | K409V | | T394W/F405A/ | |
| | | | Y407V | |
| <10% Combi | Q347E/S354Y/ | K392D/D399K/ | T394F/F405A | |
| nation 3447 | T366Y/Y407T | K409D | | |
| <10% Combi | Q347E/S354Y/ | K392D/D399K/ | K392L/T394W/ | |
| nation 3448 | T366Y/Y407T | K409D | F405A/Y407V | |
| <10% Combi | Q347E/S354Y/ | K392D/D399K/ | T350V/L351Y/ | |
| nation 3449 | T366Y/Y407T | K409D | T366L/K392L/ | |
| | | | T394W/F405A/ | |
| | | | Y407V | |
| <10% Combi | Q347E/S354Y/ | T394F/F405A | T350V/L351Y/ | |
| nation 3450 | T366Y/Y407T | | T366L/K392L/ | |
| | | | T394W/F405A/ | |
| | | | Y407V | |
| <10% Combi | K392D/D399K/ | T394F/F405A | T350V/L351Y/ | |
| nation 3451 | K409D | | T366L/K392L/ | |
| | | | T394W/F405A/ | |
| | | | Y407V | |
| <10% Combi | K392D/D399K/ | E345R/K360E/ | T394F/F405A | |
| nation 3452 | K409D | D399M/Y407A/ | | |
| | | K409V | | |
| <10% Combi | K392D/D399K/ | E345R/K360E/ | K392L/T394W/ | |
| nation 3453 | K409D | D399M/Y407A/ | F405A/Y407V | |
| | | K409V | | |
| <10% Combi | K392D/D399K/ | E345R/K360E/ | T350V/L351Y/ | |
| nation 3454 | K409D | D399M/Y407A/ | T366L/K392L/ | |
| | | K409V | T394W/F405A/ | |
| | | | Y407V | |
| <10% Combi | K392D/D399K/ | T394F/F405A | T350V/L351Y/ | |
| nation 3455 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3456 | D399M/Y407A/ | | T366L/K392L/ | |
| | K409V | | T394W/F405A/ | |
| | | | Y407V | |
| <10% Combi | E345R/K360E/ | T394F/F405A | T350V/L351Y/ | |
| nation 3457 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3458 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3459 | D399M/Y407A/ | | T366L/K392L/ | |
| | K409V | | T394W/F405A/ | |
| | | | Y407V | |
| <10% Combi | E356K/K392D/ | K392D/D399K/ | T394F/F405A | |
| nation 3460 | D399K/K439E | K409D | | |
| <10% Combi | E356K/K392D/ | K392D/D399K/ | K392L/T394W/ | |
| nation 3461 | D399K/K439E | K409D | F405A/Y407V | |
| <10% Combi | E356K/K392D/ | K392D/D399K/ | T350V/L351Y/ | |
| nation 3462 | D399K/K439E | K409D | T366L/K392L/ | |
| | | | T394W/F405A/ | |
| | | | Y407V | |
| <10% Combi | E356K/K392D/ | T394F/F405A | T350V/L351Y/ | |
| nation 3463 | D399K/K439E | | T366L/K392L/ | |
| | | | T394W/F405A/ | |
| | | | Y407V | |
| <10% Combi | K392D/D399K/ | T394F/F405A | T350V/L351Y/ | |
| nation 3464 | K409D | | T366L/K392L/ | |
| | | | T394W/F405A/ | |
| | | | Y407V | |
| <10% Combi | E356K/K392D/ | T394F/F405A | T350V/L351Y/ | |
| nation 3465 | D399K/K409D | | T366L/K392L/ | |
| | | | T394W/F405A/ | |
| | | | Y407V | |
| <10% Combi | K392D/D399K/ | T394F/F405A | T350V/L351Y/ | |
| nation 3466 | K409D | | T366L/K392L/ | |
| | | | T394W/F405A/ | |
| | | | Y407V | |
| <10% Combi | K392D/D399K/ | E345R/K360E/ | T394F/F405A | |
| nation 3467 | K409D | D399M/Y407A/ | | |
| | | K409V | | |
| <10% Combi | K392D/D399K/ | E345R/K360E/ | K392L/T394W/ | |
| nation 3468 | K409D | D399M/Y407A/ | F405A/Y407V | |
| | | K409V | | |
| <10% Combi | K392D/D399K/ | E345R/K360E/ | T350V/L351Y/ | |
| nation 3469 | K409D | D399M/Y407A/ | T366L/K392L/ | |
| | | K409V | T394W/F405A/ | |
| | | | Y407V | |
| <10% Combi | K392D/D399K/ | T394F/F405A | T350V/L351Y/ | |
| nation 3470 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3471 | D399M/Y407A/ | | T366L/K392L/ | |
| | K409V | | T394W/F405A/ | |
| | | | Y407V | |
| <10% Combi | E345R/K360E/ | T394F/F405A | T350V/L351Y/ | |
| nation 3472 | D399M/Y407A/ | | T366L/K392L/ | |
| | K409V | | T394W/F405A/ | |
| | | | Y407V | |
| <10% Combi | E345R/K360E/ | T394F/F405A | T350V/L351Y/ | |
| nation 3473 | D399M/Y407A/ | | T366L/K392L/ | |
| | K409V | | T394W/F405A/ | |
| | | | Y407V | |
| <10% Combi | E345R/K360E/ | T394F/F405A | T350V/L351Y/ | |
| nation 3474 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3475 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3476 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3477 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3478 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3479 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3480 | D399M/Y407A/ | | T366L/K392L/ | |
| | K409V | | T394W/F405A/ | |
| | | | Y407V | |
| <10% Combi | E345R/Q347R/ | K392D/D399K/ | T350V/L351Y/ | |
| nation 3481 | K360D/T366V/ | K409D | T366L/K392L/ | |
| | D399M/Y407A/ | | T394W/F405A/ | |
| | K409V | | Y407V | |
| <10% Combi | K392D/D399K/ | E345R/K360E/ | T350V/L351Y/ | |
| nation 3482 | K409D | D399M/Y407A/ | T366L/K392L/ | |
| | | K409V | T394W/F405A/ | |
| | | | Y407V | |
| <10% Combi | E356K/K392D/ | K392D/D399K/ | T350V/L351Y/ | |
| nation 3483 | D399K/K439E | K409D | T366L/K392L/ | |
| | | | T394W/F405A/ | |
| | | | Y407V | |
| <10% Combi | K392D/D399K/ | E345R/K360E/ | T350V/L351Y/ | |
| nation 3484 | K409D | D399M/Y407A/ | T366L/K392L/ | |
| | | K409V | T394W/F405A/ | |
| | | | Y407V | |
| <10% Combi | Q347E/S354Y/ | K392D/D399K/ | T350V/L351Y/ | |
| nation 3485 | T366Y/Y407T | K409D | T366L/K392L/ | |
| | | | T394W/F405A/ | |
| | | | Y407V | |
| <10% Combi | K392D/D399K/ | E345R/K360E/ | T350V/L351Y/ | |
| nation 3486 | K409D | D399M/Y407A/ | T366L/K392L/ | |
| | | K409V | T394W/F405A/ | |
| | | | Y407V | |
| <10% Combi | E356K/K392D/ | K392D/D399K/ | T350V/L351Y/ | |
| nation 3487 | D399K/K439E | K409D | T366L/K392L/ | |
| | | | T394W/F405A/ | |
| | | | Y407V | |
| <10% Combi | K392D/D399K/ | E345R/K360E/ | T350V/L351Y/ | |
| nation 3488 | K409D | D399M/Y407A/ | T366L/K392L/ | |
| | | K409V | T394W/F405A/ | |
| | | | Y407V | |
| <10% Combi | K392D/D399K/ | T394F/F405A | T350V/L351Y/ | |
| nation 3489 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3490 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3491 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3492 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3493 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3494 | D399M/Y407A/ | | T366L/K392L/ | |
| | K409V | | T394W/F405A/ | |
| | | | Y407V | |
| <10% Combi | K392D/D399K/ | T394F/F405A | T350V/L351Y/ | |
| nation 3495 | K409D | | T366L/K392L/ | |
| | | | T394W/F405A/ | |
| | | | Y407V | |
| <10% Combi | E345R/K360E/ | T394F/F405A | T350V/L351Y/ | |
| nation 3496 | D399M/Y407A/ | | T366L/K392L/ | |
| | K409V | | T394W/F405A/ | |
| | | | Y407V | |
| <10% Combi | Y349C/E357C/ | E345R/Q347R/ | K392D/D399K/ | T350V/L351Y/ |
| nation 3497 | K392D/D399K | K360D/T366V/ | K409D | T366L/K392L/ |
| | | D399M/Y407A/ | | T394W/F405A/ |
| | | K409V | | Y407V |
| <10% Combi | Y349C/E357C/ | K392D/D399K/ | E345R/K360E/ | T350V/L351Y/ |
| nation 3498 | K392D/D399K | K409D | D399M/Y407A/ | T366L/K392L/ |
| | | | K409V | T394W/F405A/ |
| | | | | Y407V |
| <10% Combi | E345R/Q347R/ | E356K/K392D/ | K392D/D399K/ | T350V/L351Y/ |
| nation 3499 | K360D/T366V/ | D399K/K439E | K409D | T366L/K392L/ |
| | D399M/Y407A/ | | | T394W/F405A/ |
| | K409V | | | Y407V |
| <10% Combi | E356K/K392D/ | K392D/D399K/ | E345R/K360E/ | T350V/L351Y/ |
| nation 3500 | D399K/K439E | K409D | D399M/Y407A/ | T366L/K392L/ |
| | | | K409V | T394W/F405A/ |
| | | | | Y407V |
| <10% Combi | E345R/Q347R/ | Q347E/S354Y/ | K392D/D399K/ | T350V/L351Y/ |
| nation 3501 | K360D/T366V/ | T366Y/Y407T | K409D | T366L/K392L/ |
| | D399M/Y407A/ | | | T394W/F405A/ |
| | K409V | | | Y407V |
| <10% Combi | Q347E/S354Y/ | K392D/D399K/ | E345R/K360E/ | T350V/L351Y/ |
| nation 3502 | T366Y/Y407T | K409D | D399M/Y407A/ | T366L/K392L/ |
| | | | K409V | T394W/F405A/ |
| | | | | Y407V |
| <10% Combi | Q347E/S354Y/ | E356K/K392D/ | K392D/D399K/ | T350V/L351Y/ |
| nation 3503 | T366Y/Y407T | D399K/K439E | K409D | T366L/K392L/ |
| | | | | T394W/F405A/ |
| | | | | Y407V |
| <10% Combi | E356K/K392D/ | K392D/D399K/ | E345R/K360E/ | T350V/L351Y/ |
| nation 3504 | D399K/K439E | K409D | D399M/Y407A/ | T366L/K392L/ |
| | | | K409V | T394W/F405A/ |
| | | | | Y407V |
| <10% Combi | E345R/Q347R/ | K392D/D399K/ | T394F/F405A | T350V/L351Y/ |
| nation 3505 | K360D/T366V/ | K409D | | T366L/K392L/ |
| | D399M/Y407A/ | | | T394W/F405A/ |
| | K409V | | | Y407V |
| <10% Combi | K392D/D399K/ | E345R/K360E/ | T394F/F405A | T350V/L351Y/ |
| nation 3506 | K409D | D399M/Y407A/ | | T366L/K392L/ |
| | | K409V | | T394W/F405A/ |
| | | | | Y407V |
| <10% Combi | E356K/K392D/ | K392D/D399K/ | T394F/F405A | T350V/L351Y/ |
| nation 3507 | D399K/K439E | K409D | | T366L/K392L/ |
| | | | | T394W/F405A/ |
| | | | | Y407V |
| <10% Combi | K392D/D399K/ | E345R/K360E/ | T394F/F405A | T350V/L351Y/ |
| nation 3508 | K409D | D399M/Y407A/ | | T366L/K392L/ |
| | | K409V | | T394W/F405A/ |
| | | | | Y407V |
| <10% Combi | Q347E/S354Y/ | K392D/D399K/ | T394F/F405A | T350V/L351Y/ |
| nation 3509 | T366Y/Y407T | K409D | | T366L/K392L/ |
| | | | | T394W/F405A/ |
| | | | | Y407V |
| <10% Combi | K392D/D399K/ | E345R/K360E/ | T394F/F405A | T350V/L351Y/ |
| nation 3510 | K409D | D399M/Y407A/ | | T366L/K392L/ |
| | | K409V | | T394W/F405A/ |
| | | | | Y407V |
| <10% Combi | E356K/K392D/ | K392D/D399K/ | T394F/F405A | T350V/L351Y/ |
| nation 3511 | D399K/K439E | K409D | | T366L/K392L/ |
| | | | | T394W/F405A/ |
| | | | | Y407V |
| <10% Combi | K392D/D399K/ | E345R/K360E/ | T394F/F405A | T350V/L351Y/ |
| nation 3512 | K409D | D399M/Y407A/ | | T366L/K392L/ |
| | | K409V | | T394W/F405A/ |
| | | | | Y407V |

TABLE 7
Amino acid modification pairs that can be used for coexpression of two to six antibodies (composed
of amino acid modification pairs that showed a heteromultimer level of 0% in Table 5)

| Amino acid | | | | | | |
|---|---|---|---|---|---|---|
| modification pairs | Antibody 1 | Antibody 2 | Antibody 3 | Antibody 4 | Antibody 5 | Antibody 6 |
| 0% Combination 1 | WT | T394W/F405A | | | | |
| 0% Combination 2 | WT | T366W/L368A/ | | | | |
| | | Y407V | | | | |
| 0% Combination 3 | WT | Q347R/K360E/ | | | | |
| | | D399V/F405T/ | | | | |
| | | K409W | | | | |
| 0% Combination 4 | WT | E356K/D399K/ | | | | |
| | | K409E/K439E | | | | |
| 0% Combination 5 | WT | Y349C/E357C/ | | | | |
| | | K392D/D399K | | | | |
| 0% Combination 6 | WT | E345R/Q347R/ | | | | |
| | | K360D/T366V/ | | | | |
| | | D399M/Y407A/ | | | | |
| | | K409V | | | | |
| 0% Combination 7 | WT | Q347E/S354Y/ | | | | |
| | | T366Y/Y407T | | | | |
| 0% Combination 8 | WT | E356K/K392D/ | | | | |
| | | D399K/K439E | | | | |
| 0% Combination 9 | WT | K392D/D399K/ | | | | |
| | | K409D | | | | |
| 0% Combination 10 | WT | E356K/K392D/ | | | | |
| | | D399K/K409D | | | | |
| 0% Combination 11 | WT | E345R/K360E/ | | | | |
| | | D399M/Y407A/ | | | | |
| | | K409V | | | | |
| 0% Combination 12 | WT | T394F/F405A | | | | |
| 0% Combination 13 | WT | K392L/T394W/ | | | | |
| | | F405A/Y407V | | | | |
| 0% Combination 14 | WT | T350V/L351Y/ | | | | |
| | | T366L/K392L/ | | | | |
| | | T394W/F405A/ | | | | |
| | | Y407V | | | | |
| 0% Combination 15 | T394W/F405A | T366W/L368A/ | | | | |
| | | Y407V | | | | |
| 0% Combination 16 | T394W/F405A | Q347R/K360E/ | | | | |
| | | D399V/F405T/ | | | | |
| | | K409W | | | | |
| 0% Combination 17 | T394W/F405A | E356K/D399K/ | | | | |
| | | K409E/K439E | | | | |
| 0% Combination 18 | T394W/F405A | Y349C/E357C/ | | | | |
| | | K392D/D399K | | | | |
| 0% Combination 19 | T394W/F405A | E345R/Q347R/ | | | | |
| | | K360D/T366V/ | | | | |
| | | D399M/Y407A/ | | | | |
| | | K409V | | | | |
| 0% Combination 20 | T394W/F405A | Q347E/S354Y/ | | | | |
| | | T366Y/Y407T | | | | |
| 0% Combination 21 | T394W/F405A | E356K/K392D/ | | | | |
| | | D399K/K439E | | | | |
| 0% Combination 22 | T394W/F405A | K392D/D399K/ | | | | |
| | | K409D | | | | |
| 0% Combination 23 | T394W/F405A | E356K/K392D/ | | | | |
| | | D399K/K409D | | | | |
| 0% Combination 24 | T394W/F405A | E345R/K360E/ | | | | |
| | | D399M/Y407A/ | | | | |
| | | K409V | | | | |
| 0% Combination 25 | T366W/L368A/ | Q347R/K360E/ | | | | |
| | Y407V | D399V/F405T/ | | | | |
| | | K409W | | | | |
| 0% Combination 26 | T366W/L368A/ | E356K/D399K/ | | | | |
| | Y407V | K409E/K439E | | | | |
| 0% Combination 27 | T366W/L368A/ | Y349C/E357C/ | | | | |
| | Y407V | K392D/D399K | | | | |
| 0% Combination 28 | T366W/L368A/ | E345R/Q347R/ | | | | |
| | Y407V | K360D/T366V/ | | | | |
| | | D399M/Y407A/ | | | | |
| | | K409V | | | | |
| 0% Combination 29 | T366W/L368A/ | E356K/K392D/ | | | | |
| | Y407V | D399K/K439E | | | | |
| 0% Combination 30 | T366W/L368A/ | K392D/D399K/ | | | | |
| | Y407V | K409D | | | | |
| 0% Combination 31 | T366W/L368A/ | E356K/K392D/ | | | | |
| | Y407V | D399K/K409D | | | | |
| 0% Combination 32 | T366W/L368A/ | E345R/K360E/ | | | | |
| | Y407V | D399M/Y407A/ | | | | |
| | | K409V | | | | |
| 0% Combination 33 | T366W/L368A/ | T394F/F405A | | | | |
| | Y407V | | | | | |
| 0% Combination 34 | T366W/L368A/ | K392L/T394W/ | | | | |
| | Y407V | F405A/Y407V | | | | |
| 0% Combination 35 | T366W/L368A/ | T350V/L351Y/ | | | | |
| | Y407V | T366L/K392L/ | | | | |
| | | T394W/F405A/ | | | | |
| | | Y407V | | | | |
| 0% Combination 36 | Q347R/K360E/ | E356K/D399K/ | | | | |
| | D399V/F405T/ | K409E/K439E | | | | |
| | K409W | | | | | |
| 0% Combination 37 | Q347R/K360E/ | Y349C/E357C/ | | | | |
| | D399V/F405T/ | K392D/D399K | | | | |
| | K409W | | | | | |
| 0% Combination 56 | Y349C/E357C/ | K392L/T394W/ | | | | |
| | K392D/D399K | F405A/Y407V | | | | |
| 0% Combination 57 | Y349C/E357C/ | T350V/L351Y/ | | | | |
| | K392D/D399K | T366L/K392L/ | | | | |
| | | T394W/F405A/ | | | | |
| | | Y407V | | | | |
| 0% Combination 58 | E345R/Q347R/ | E356K/K392D/ | | | | |
| | K360D/T366V/ | D399K/K439E | | | | |
| | D399M/Y407A/ | | | | | |
| | K409V | | | | | |
| 0% Combination 59 | E345R/Q347R/ | K392D/D399K/ | | | | |
| | K360D/T366V/ | K409D | | | | |
| | D399M/Y407A/ | | | | | |
| | K409V | | | | | |
| 0% Combination 60 | E345R/Q347R/ | E356K/K392D/ | | | | |
| | K360D/T366V/ | D399K/K409D | | | | |
| | D399M/Y407A/ | | | | | |
| | K409V | | | | | |
| 0% Combination 61 | E345R/Q347R/ | T394F/F405A | | | | |
| | K360D/T366V/ | | | | | |
| | D399M/Y407A/ | | | | | |
| | K409V | | | | | |
| 0% Combination 62 | E345R/Q347R/ | K392L/T394W/ | | | | |
| | K360D/T366V/ | F405A/Y407V | | | | |
| | D399M/Y407A/ | | | | | |
| | K409V | | | | | |
| 0% Combination 63 | E345R/Q347R/ | T350V/L351Y/ | | | | |
| | K360D/T366V/ | T366L/K392L/ | | | | |
| | D399M/Y407A/ | T394W/F405A/ | | | | |
| | K409V | Y407V | | | | |
| 0% Combination 64 | Q347E/S354Y/ | E356K/K392D/ | | | | |
| | T366Y/Y407T | D399K/K439E | | | | |
| 0% Combination 65 | Q347E/S354Y/ | K392D/D399K/ | | | | |
| | T366Y/Y407T | K409D | | | | |
| 0% Combination 66 | Q347E/S354Y/ | E356K/K392D/ | | | | |
| | T366Y/Y407T | D399K/K409D | | | | |
| 0% Combination 67 | Q347E/S354Y/ | E345R/K360E/ | | | | |
| | T366Y/Y407T | D399M/Y407A/ | | | | |
| | | K409V | | | | |
| 0% Combination 68 | Q347E/S354Y/ | T394F/F405A | | | | |
| | T366Y/Y407T | | | | | |
| 0% Combination 69 | Q347E/S354Y/ | K392L/T394W/ | | | | |
| | T366Y/Y407T | F405A/Y407V | | | | |
| 0% Combination 70 | Q347E/S354Y/ | T350V/L351Y/ | | | | |
| | T366Y/Y407T | T366L/K392L/ | | | | |
| | | T394W/F405A/ | | | | |
| | | Y407V | | | | |
| 0% Combination 71 | E356K/K392D/ | E345R/K360E/ | | | | |
| | D399K/K439E | D399M/Y407A/ | | | | |
| | | K409V | | | | |
| 0% Combination 72 | E356K/K392D/ | T394F/F405A | | | | |
| | D399K/K439E | | | | | |
| 0% Combination 73 | E356K/K392D/ | K392L/T394W/ | | | | |
| | D399K/K439E | F405A/Y407V | | | | |
| 0% Combination 74 | E356K/K392D/ | T350V/L351Y/ | | | | |
| | D399K/K439E | T366L/K392L/ | | | | |
| | | T394W/F405A/ | | | | |
| | | Y407V | | | | |
| 0% Combination 75 | K392D/D399K/ | T394F/F405A | | | | |
| | K409D | | | | | |
| 0% Combination 76 | K392D/D399K/ | K392L/T394W/ | | | | |
| | K409D | F405A/Y407V | | | | |
| 0% Combination 77 | K392D/D399K/ | T350V/L351Y/ | | | | |
| | K409D | T366L/K392L/ | | | | |
| | | T394W/F405A/ | | | | |
| | | Y407V | | | | |
| 0% Combination 78 | E356K/K392D/ | E345R/K360E/ | | | | |
| | D399K/K409D | D399M/Y407A/ | | | | |
| | | K409V | | | | |
| 0% Combination 79 | E356K/K392D/ | T394F/F405A | | | | |
| | D399K/K409D | | | | | |
| 0% Combination 80 | E356K/K392D/ | K392L/T394W/ | | | | |
| | D399K/K409D | F405A/Y407V | | | | |
| 0% Combination 81 | E356K/K392D/ | T350V/L351Y/ | | | | |
| | D399K/K409D | T366L/K392L/ | | | | |
| | | T394W/F405A/ | | | | |
| | | Y407V | | | | |
| 0% Combination 82 | E345R/K360E/ | T394F/F405A | | | | |
| | D399M/Y407A/ | | | | | |
| | K409V | | | | | |
| 0% Combination 83 | E345R/K360E/ | K392L/T394W/ | | | | |
| | D399M/Y407A/ | F405A/Y407V | | | | |
| | K409V | | | | | |
| 0% Combination 84 | E345R/K360E/ | T350V/L351Y/ | | | | |
| | D399M/Y407A/ | T366L/K392L/ | | | | |
| | K409V | T394W/F405A/ | | | | |
| | | Y407V | | | | |
| 0% Combination 85 | WT | T394W/F405A | T366W/L368A/ | | | |
| | | | Y407V | | | |
| 0% Combination 86 | WT | T394W/F405A | Q347R/K360E/ | | | |
| | | | D399V/F405T/ | | | |
| | | | K409W | | | |
| 0% Combination 87 | WT | T394W/F405A | E356K/D399K/ | | | |
| | | | K409E/K439E | | | |
| 0% Combination 88 | WT | T394W/F405A | Y349C/E357C/ | | | |
| | | | K392D/D399K | | | |
| 0% Combination 89 | WT | T394W/F405A | E345R/Q347R/ | | | |
| | | | K360D/T366V/ | | | |
| | | | D399M/Y407A/ | | | |
| | | | K409V | | | |
| 0% Combination 90 | WT | T394W/F405A | Q347E/S354Y/ | | | |
| | | | T366Y/Y407T | | | |
| 0% Combination 91 | WT | T394W/F405A | E356K/K392D/ | | | |
| | | | D399K/K439E | | | |
| 0% Combination 92 | WT | T394W/F405A | K392D/D399K/ | | | |
| | | | K409D | | | |
| 0% Combination 93 | WT | T394W/F405A | E356K/K392D/ | | | |
| | | | D399K/K409D | | | |
| 0% Combination 94 | WT | T394W/F405A | E345R/K360E/ | | | |
| | | | D399M/Y407A/ | | | |
| | | | K409V | | | |
| 0% Combination 95 | WT | T366W/L368A/ | Q347R/K360E/ | | | |
| | | Y407V | D399V/F405T/ | | | |
| | | | K409W | | | |
| 0% Combination 96 | WT | T366W/L368A/ | E356K/D399K/ | | | |
| | | Y407V | K409E/K439E | | | |
| 0% Combination 97 | WT | T366W/L368A/ | Y349C/E357C/ | | | |
| | | Y407V | K392D/D399K | | | |
| 0% Combination 98 | WT | T366W/L368A/ | E345R/Q347R/ | | | |
| | | Y407V | K360D/T366V/ | | | |
| | | | D399M/Y407A/ | | | |
| | | | K409V | | | |
| 0% Combination 99 | WT | T366W/L368A/ | E356K/K392D/ | | | |
| | | Y407V | D399K/K439E | | | |
| 0% Combination 100 | WT | T366W/L368A/ | K392D/D399K/ | | | |
| | | Y407V | K409D | | | |
| 0% Combination 101 | WT | T366W/L368A/ | E356K/K392D/ | | | |
| | | Y407V | D399K/K409D | | | |
| 0% Combination 102 | WT | T366W/L368A/ | E345R/K360E/ | | | |
| | | Y407V | D399M/Y407A/ | | | |
| | | | K409V | | | |
| 0% Combination 103 | WT | T366W/L368A/ | T394F/F405A | | | |
| | | Y407V | | | | |
| 0% Combination 104 | WT | T366W/L368A/ | K392L/T394W/ | | | |
| | | Y407V | F405A/Y407V | | | |
| 0% Combination 105 | WT | T366W/L368A/ | T350V/L351Y/ | | | |
| | | Y407V | T366L/K392L/ | | | |
| | | | T394W/F405A/ | | | |
| | | | Y407V | | | |
| 0% Combination 56 | Y349C/E357C/ | K392L/T394W/ | | | | |
| | K392D/D399K | F405A/Y407V | | | | |
| 0% Combination 57 | Y349C/E357C/ | T350V/L351Y/ | | | | |
| | K392D/D399K | T366L/K392L/ | | | | |
| | | T394W/F405A/ | | | | |
| | | Y407V | | | | |
| 0% Combination 58 | E345R/Q347R/ | E356K/K392D/ | | | | |
| | K360D/T366V/ | D399K/K439E | | | | |
| | D399M/Y407A/ | | | | | |
| | K409V | | | | | |
| 0% Combination 59 | E345R/Q347R/ | K392D/D399K/ | | | | |
| | K360D/T366V/ | K409D | | | | |
| | D399M/Y407A/ | | | | | |
| | K409V | | | | | |
| 0% Combination 60 | E345R/Q347R/ | E356K/K392D/ | | | | |
| | K360D/T366V/ | D399K/K409D | | | | |
| | D399M/Y407A/ | | | | | |
| | K409V | | | | | |
| 0% Combination 61 | E345R/Q347R/ | T394F/F405A | | | | |
| | K360D/T366V/ | | | | | |
| | D399M/Y407A/ | | | | | |
| | K409V | | | | | |
| 0% Combination 62 | E345R/Q347R/ | K392L/T394W/ | | | | |
| | K360D/T366V/ | F405A/Y407V | | | | |
| | D399M/Y407A/ | | | | | |
| | K409V | | | | | |
| 0% Combination 63 | E345R/Q347R/ | T350V/L351Y/ | | | | |
| | K360D/T366V/ | T366L/K392L/ | | | | |
| | D399M/Y407A/ | T394W/F405A/ | | | | |
| | K409V | Y407V | | | | |
| 0% Combination 64 | Q347E/S354Y/ | E356K/K392D/ | | | | |
| | T366Y/Y407T | D399K/K439E | | | | |
| 0% Combination 65 | Q347E/S354Y/ | K392D/D399K/ | | | | |
| | T366Y/Y407T | K409D | | | | |
| 0% Combination 66 | Q347E/S354Y/ | E356K/K392D/ | | | | |
| | T366Y/Y407T | D399K/K409D | | | | |
| 0% Combination 67 | Q347E/S354Y/ | E345R/K360E/ | | | | |
| | T366Y/Y407T | D399M/Y407A/ | | | | |
| | | K409V | | | | |
| 0% Combination 68 | Q347E/S354Y/ | T394F/F405A | | | | |
| | T366Y/Y407T | | | | | |
| 0% Combination 69 | Q347E/S354Y/ | K392L/T394W/ | | | | |
| | T366Y/Y407T | F405A/Y407V | | | | |
| 0% Combination 70 | Q347E/S354Y/ | T350V/L351Y/ | | | | |
| | T366Y/Y407T | T366L/K392L/ | | | | |
| | | T394W/F405A/ | | | | |
| | | Y407V | | | | |
| 0% Combination 71 | E356K/K392D/ | E345R/K360E/ | | | | |
| | D399K/K439E | D399M/Y407A/ | | | | |
| | | K409V | | | | |
| 0% Combination 72 | E356K/K392D/ | T394F/F405A | | | | |
| | D399K/K439E | | | | | |
| 0% Combination 73 | E356K/K392D/ | K392L/T394W/ | | | | |
| | D399K/K439E | F405A/Y407V | | | | |
| 0% Combination 74 | E356K/K392D/ | T350V/L351Y/ | | | | |
| | D399K/K439E | T366L/K392L/ | | | | |
| | | T394W/F405A/ | | | | |
| | | Y407V | | | | |
| 0% Combination 75 | K392D/D399K/ | T394F/F405A | | | | |
| | K409D | | | | | |
| 0% Combination 76 | K392D/D399K/ | K392L/T394W/ | | | | |
| | K409D | F405A/Y407V | | | | |
| 0% Combination 77 | K392D/D399K/ | T350V/L351Y/ | | | | |
| | K409D | T366L/K392L/ | | | | |
| | | T394W/F405A/ | | | | |
| | | Y407V | | | | |
| 0% Combination 78 | E356K/K392D/ | E345R/K360E/ | | | | |
| | D399K/K409D | D399M/Y407A/ | | | | |
| | | K409V | | | | |
| 0% Combination 79 | E356K/K392D/ | T394F/F405A | | | | |
| | D399K/K409D | | | | | |
| 0% Combination 80 | E356K/K392D/ | K392L/T394W/ | | | | |
| | D399K/K409D | F405A/Y407V | | | | |
| 0% Combination 81 | E356K/K392D/ | T350V/L351Y/ | | | | |
| | D399K/K409D | T366L/K392L/ | | | | |
| | | T394W/F405A/ | | | | |
| | | Y407V | | | | |
| 0% Combination 82 | E345R/K360E/ | T394F/F405A | | | | |
| | D399M/Y407A/ | | | | | |
| | K409V | | | | | |
| 0% Combination 83 | E345R/K360E/ | K392L/T394W/ | | | | |
| | D399M/Y407A/ | F405A/Y407V | | | | |
| | K409V | | | | | |
| 0% Combination 84 | E345R/K360E/ | T350V/L351Y/ | | | | |
| | D399M/Y407A/ | T366L/K392L/ | | | | |
| | K409V | T394W/F405A/ | | | | |
| | | Y407V | | | | |
| 0% Combination 85 | WT | T394W/F405A | T366W/L368A/ | | | |
| | | | Y407V | | | |
| 0% Combination 86 | WT | T394W/F405A | Q347R/K360E/ | | | |
| | | | D399V/F405T/ | | | |
| | | | K409W | | | |
| 0% Combination 87 | WT | T394W/F405A | E356K/D399K/ | | | |
| | | | K409E/K439E | | | |
| 0% Combination 88 | WT | T394W/F405A | Y349C/E357C/ | | | |
| | | | K392D/D399K | | | |
| 0% Combination 89 | WT | T394W/F405A | E345R/Q347R/ | | | |
| | | | K360D/T366V/ | | | |
| | | | D399M/Y407A/ | | | |
| | | | K409V | | | |
| 0% Combination 90 | WT | T394W/F405A | Q347E/S354Y/ | | | |
| | | | T366Y/Y407T | | | |
| 0% Combination 91 | WT | T394W/F405A | E356K/K392D/ | | | |
| | | | D399K/K439E | | | |
| 0% Combination 92 | WT | T394W/F405A | K392D/D399K/ | | | |
| | | | K409D | | | |
| 0% Combination 93 | WT | T394W/F405A | E356K/K392D/ | | | |
| | | | D399K/K409D | | | |
| 0% Combination 94 | WT | T394W/F405A | E345R/K360E/ | | | |
| | | | D399M/Y407A/ | | | |
| | | | K409V | | | |
| 0% Combination 95 | WT | T366W/L368A/ | Q347R/K360E/ | | | |
| | | Y407V | D399V/F405T/ | | | |
| | | | K409W | | | |
| 0% Combination 96 | WT | T366W/L368A/ | E356K/D399K/ | | | |
| | | Y407V | K409E/K439E | | | |
| 0% Combination 97 | WT | T366W/L368A/ | Y349C/E357C/ | | | |
| | | Y407V | K392D/D399K | | | |
| 0% Combination 98 | WT | T366W/L368A/ | E345R/Q347R/ | | | |
| | | Y407V | K360D/T366V/ | | | |
| | | | D399M/Y407A/ | | | |
| | | | K409V | | | |
| 0% Combination 99 | WT | T366W/L368A/ | E356K/K392D/ | | | |
| | | Y407V | D399K/K439E | | | |
| 0% Combination 100 | WT | T366W/L368A/ | K392D/D399K/ | | | |
| | | Y407V | K409D | | | |
| 0% Combination 101 | WT | T366W/L368A/ | E356K/K392D/ | | | |
| | | Y407V | D399K/K409D | | | |
| 0% Combination 102 | WT | T366W/L368A/ | E345R/K360E/ | | | |
| | | Y407V | D399M/Y407A/ | | | |
| | | | K409V | | | |
| 0% Combination 103 | WT | T366W/L368A/ | T394F/F405A | | | |
| | | Y407V | | | | |
| 0% Combination 104 | WT | T366W/L368A/ | K392L/T394W/ | | | |
| | | Y407V | F405A/Y407V | | | |
| 0% Combination 105 | WT | T366W/L368A/ | T350V/L351Y/ | | | |
| | | Y407V | T366L/K392L/ | | | |
| | | | T394W/F405A/ | | | |
| | | | Y407V | | | |
| 0% Combination 106 | WT | Q347R/K360E/ | E356K/D399K/ | | | |
| | | D399V/F405T/ | K409E/K439E | | | |
| | | K409W | | | | |
| 0% Combination 107 | WT | Q347R/K360E/ | Y349C/E357C/ | | | |
| | | D399V/F405T/ | K392D/D399K | | | |
| | | K409W | | | | |
| 0% Combination 108 | WT | Q347R/K360E/ | Q347E/S354Y/ | | | |
| | | D399V/F405T/ | T366Y/Y407T | | | |
| | | K409W | | | | |
| 0% Combination 109 | WT | Q347R/K360E/ | E356K/K392D/ | | | |
| | | D399V/F405T/ | D399K/K439E | | | |
| | | K409W | | | | |
| 0% Combination 110 | WT | Q347R/K360E/ | K392D/D399K/ | | | |
| | | D399V/F405T/ | K409D | | | |
| | | K409W | | | | |
| 0% Combination 111 | WT | Q347R/K360E/ | E356K/K392D/ | | | |
| | | D399V/F405T/ | D399K/K409D | | | |
| | | K409W | | | | |
| 0% Combination 112 | WT | Q347R/K360E/ | T394F/F405A | | | |
| | | D399V/F405T/ | | | | |
| | | K409W | | | | |
| 0% Combination 113 | WT | Q347R/K360E/ | K392L/T394W/ | | | |
| | | D399V/F405T/ | F405A/Y407V | | | |
| | | K409W | | | | |
| 0% Combination 114 | WT | Q347R/K360E/ | T350V/L351Y/ | | | |
| | | D399V/F405T/ | T366L/K392L/ | | | |
| | | K409W | T394W/F405A/ | | | |
| | | | Y407V | | | |
| 0% Combination 115 | WT | E356K/D399K/ | Y349C/E357C/ | | | |
| | | K409E/K439E | K392D/D399K | | | |
| 0% Combination 116 | WT | E356K/D399K/ | E345R/Q347R/ | | | |
| | | K409E/K439E | K360D/T366V/ | | | |
| | | | D399M/Y407A/ | | | |
| | | | K409V | | | |
| 0% Combination 117 | WT | E356K/D399K/ | Q347E/S354Y/ | | | |
| | | K409E/K439E | T366Y/Y407T | | | |
| 0% Combination 118 | WT | E356K/D399K/ | E345R/K360E/ | | | |
| | | K409E/K439E | D399M/Y407A/ | | | |
| | | | K409V | | | |
| 0% Combination 119 | WT | E356K/D399K/ | T394F/F405A | | | |
| | | K409E/K439E | | | | |
| 0% Combination 120 | WT | E356K/D399K/ | K392L/T394W/ | | | |
| | | K409E/K439E | F405A/Y407V | | | |
| 0% Combination 121 | WT | E356K/D399K/ | T350V/L351Y/ | | | |
| | | K409E/K439E | T366L/K392L/ | | | |
| | | | T394W/F405A/ | | | |
| | | | Y407V | | | |
| 0% Combination 122 | WT | Y349C/E357C/ | E356K/K392D/ | | | |
| | | K392D/D399K | D399K/K439E | | | |
| 0% Combination 123 | WT | Y349C/E357C/ | E356K/K392D/ | | | |
| | | K392D/D399K | D399K/K409D | | | |
| 0% Combination 124 | WT | Y349C/E357C/ | E345R/K360E/ | | | |
| | | K392D/D399K | D399M/Y407A/ | | | |
| | | | K409V | | | |
| 0% Combination 125 | WT | Y349C/E357C/ | T394F/F405A | | | |
| | | K392D/D399K | | | | |
| 0% Combination 126 | WT | Y349C/E357C/ | K392L/T394W/ | | | |
| | | K392D/D399K | F405A/Y407V | | | |
| 0% Combination 127 | WT | Y349C/E357C/ | T350V/L351Y/ | | | |
| | | K392D/D399K | T366L/K392L/ | | | |
| | | | T394W/F405A/ | | | |
| | | | Y407V | | | |
| 0% Combination 128 | WT | E345R/Q347R/ | E356K/K392D/ | | | |
| | | K360D/T366V/ | D399K/K439E | | | |
| | | D399M/Y407A/ | | | | |
| | | K409V | | | | |
| 0% Combination 129 | WT | E345R/Q347R/ | K392D/D399K/ | | | |
| | | K360D/T366V/ | K409D | | | |
| | | D399M/Y407A/ | | | | |
| | | K409V | | | | |
| 0% Combination 130 | WT | E345R/Q347R/ | E356K/K392D/ | | | |
| | | K360D/T366V/ | D399K/K409D | | | |
| | | D399M/Y407A/ | | | | |
| | | K409V | | | | |
| 0% Combination 131 | WT | E345R/Q347R/ | T394F/F405A | | | |
| | | K360D/T366V/ | | | | |
| | | D399M/Y407A/ | | | | |
| | | K409V | | | | |
| 0% Combination 132 | WT | E345R/Q347R/ | K392L/T394W/ | | | |
| | | K360D/T366V/ | F405A/Y407V | | | |
| | | D399M/Y407A/ | | | | |
| | | K409V | | | | |
| 0% Combination 133 | WT | E345R/Q347R/ | T350V/L351Y/ | | | |
| | | K360D/T366V/ | T366L/K392L/ | | | |
| | | D399M/Y407A/ | T394W/F405A/ | | | |
| | | K409V | Y407V | | | |
| 0% Combination 134 | WT | Q347E/S354Y/ | E356K/K392D/ | | | |
| | | T366Y/Y407T | D399K/K439E | | | |
| 0% Combination 135 | WT | Q347E/S354Y/ | K392D/D399K/ | | | |
| | | T366Y/Y407T | K409D | | | |
| 0% Combination 136 | WT | Q347E/S354Y/ | E356K/K392D/ | | | |
| | | T366Y/Y407T | D399K/K409D | | | |
| 0% Combination 137 | WT | Q347E/S354Y/ | E345R/K360E/ | | | |
| | | T366Y/Y407T | D399M/Y407A/ | | | |
| | | | K409V | | | |
| 0% Combination 138 | WT | Q347E/S354Y/ | T394F/F405A | | | |
| | | T366Y/Y407T | | | | |
| 0% Combination 139 | WT | Q347E/S354Y/ | K392L/T394W/ | | | |
| | | T366Y/Y407T | F405A/Y407V | | | |
| 0% Combination 140 | WT | Q347E/S354Y/ | T350V/L351Y/ | | | |
| | | T366Y/Y407T | T366L/K392L/ | | | |
| | | | T394W/F405A/ | | | |
| | | | Y407V | | | |
| 0% Combination 141 | WT | E356K/K392D/ | E345R/K360E/ | | | |
| | | D399K/K439E | D399M/Y407A/ | | | |
| | | | K409V | | | |
| 0% Combination 142 | WT | E356K/K392D/ | T394F/F405A | | | |
| | | D399K/K439E | | | | |
| 0% Combination 143 | WT | E356K/K392D/ | K392L/T394W/ | | | |
| | | D399K/K439E | F405A/Y407V | | | |
| 0% Combination 144 | WT | E356K/K392D/ | T350V/L351Y/ | | | |
| | | D399K/K439E | T366L/K392L/ | | | |
| | | | T394W/F405A/ | | | |
| | | | Y407V | | | |
| 0% Combination 145 | WT | K392D/D399K/ | T394F/F405A | | | |
| | | K409D | | | | |
| 0% Combination 146 | WT | K392D/D399K/ | K392L/T394W/ | | | |
| | | K409D | F405A/Y407V | | | |
| 0% Combination 147 | WT | K392D/D399K/ | T350V/L351Y/ | | | |
| | | K409D | T366L/K392L/ | | | |
| | | | T394W/F405A/ | | | |
| | | | Y407V | | | |
| 0% Combination 148 | WT | E356K/K392D/ | E345R/K360E/ | | | |
| | | D399K/K409D | D399M/Y407A/ | | | |
| | | | K409V | | | |
| 0% Combination 149 | WT | E356K/K392D/ | T394F/F405A | | | |
| | | D399K/K409D | | | | |
| 0% Combination 150 | WT | E356K/K392D/ | K392L/T394W/ | | | |
| | | D399K/K409D | F405A/Y407V | | | |
| 0% Combination 151 | WT | E356K/K392D/ | T350V/L351Y/ | | | |
| | | D399K/K409D | T366L/K392L/ | | | |
| | | | T394W/F405A/ | | | |
| | | | Y407V | | | |
| 0% Combination 152 | WT | E345R/K360E/ | T394F/F405A | | | |
| | | D399M/Y407A/ | | | | |
| | | K409V | | | | |
| 0% Combination 153 | WT | E345R/K360E/ | K392L/T394W/ | | | |
| | | D399M/Y407A/ | F405A/Y407V | | | |
| | | K409V | | | | |
| 0% Combination 154 | WT | E345R/K360E/ | T350V/L351Y/ | | | |
| | | D399M/Y407A/ | T366L/K392L/ | | | |
| | | K409V | T394W/F405A/ | | | |
| | | | Y407V | | | |
| 0% Combination 155 | T394W/F405A | T366W/L368A/ | Q347R/K360E/ | | | |
| | | Y407V | D399V/F405T/ | | | |
| | | | K409W | | | |
| 0% Combination 156 | T394W/F405A | T366W/L368A/ | E356K/D399K/ | | | |
| | | Y407V | K409E/K439E | | | |
| 0% Combination 157 | T394W/F405A | T366W/L368A/ | Y349C/E357C/ | | | |
| | | Y407V | K392D/D399K | | | |
| 0% Combination 158 | T394W/F405A | T366W/L368A/ | E345R/Q347R/ | | | |
| | | Y407V | K360D/T366V/ | | | |
| | | | D399M/Y407A/ | | | |
| | | | K409V | | | |
| 0% Combination 159 | T394W/F405A | T366W/L368A/ | E356K/K392D/ | | | |
| | | Y407V | D399K/K439E | | | |
| 0% Combination 160 | T394W/F405A | T366W/L368A/ | K392D/D399K/ | | | |
| | | Y407V | K409D | | | |
| 0% Combination 161 | T394W/F405A | T366W/L368A/ | E356K/K392D/ | | | |
| | | Y407V | D399K/K409D | | | |
| 0% Combination 162 | T394W/F405A | T366W/L368A/ | E345R/K360E/ | | | |
| | | Y407V | D399M/Y407A/ | | | |
| | | | K409V | | | |
| 0% Combination 163 | T394W/F405A | Q347R/K360E/ | E356K/D399K/ | | | |
| | | D399V/F405T/ | K409E/K439E | | | |
| | | K409W | | | | |
| 0% Combination 164 | T394W/F405A | Q347R/K360E/ | Y349C/E357C/ | | | |
| | | D399V/F405T/ | K392D/D399K | | | |
| | | K409W | | | | |
| 0% Combination 165 | T394W/F405A | Q347R/K360E/ | Q347E/S354Y/ | | | |
| | | D399V/F405T/ | T366Y/Y407T | | | |
| | | K409W | | | | |
| 0% Combination 166 | T394W/F405A | Q347R/K360E/ | E356K/K392D/ | | | |
| | | D399V/F405T/ | D399K/K439E | | | |
| | | K409W | | | | |
| 0% Combination 167 | T394W/F405A | Q347R/K360E/ | K392D/D399K/ | | | |
| | | D399V/F405T/ | K409D | | | |
| | | K409W | | | | |
| 0% Combination 168 | T394W/F405A | Q347R/K360E/ | E356K/K392D/ | | | |
| | | D399V/F405T/ | D399K/K409D | | | |
| | | K409W | | | | |
| 0% Combination 169 | T394W/F405A | E356K/D399K/ | Y349C/E357C/ | | | |
| | | K409E/K439E | K392D/D399K | | | |
| 0% Combination 170 | T394W/F405A | E356K/D399K/ | E345R/Q347R/ | | | |
| | | K409E/K439E | K360D/T366V/ | | | |
| | | | D399M/Y407A/ | | | |
| | | | K409V | | | |
| 0% Combination 171 | T394W/F405A | E356K/D399K/ | Q347E/S354Y/ | | | |
| | | K409E/K439E | T366Y/Y407T | | | |
| 0% Combination 172 | T394W/F405A | E356K/D399K/ | E345R/K360E/ | | | |
| | | K409E/K439E | D399M/Y407A/ | | | |
| | | | K409V | | | |
| 0% Combination 173 | T394W/F405A | Y349C/E357C/ | E356K/K392D/ | | | |
| | | K392D/D399K | D399K/K439E | | | |
| 0% Combination 174 | T394W/F405A | Y349C/E357C/ | E356K/K392D/ | | | |
| | | K392D/D399K | D399K/K409D | | | |
| 0% Combination 175 | T394W/F405A | Y349C/E357C/ | E345R/K360E/ | | | |
| | | K392D/D399K | D399M/Y407A/ | | | |
| | | | K409V | | | |
| 0% Combination 176 | T394W/F405A | E345R/Q347R/ | E356K/K392D/ | | | |
| | | K360D/T366V/ | D399K/K439E | | | |
| | | D399M/Y407A/ | | | | |
| | | K409V | | | | |
| 0% Combination 177 | T394W/F405A | E345R/Q347R/ | K392D/D399K/ | | | |
| | | K360D/T366V/ | K409D | | | |
| | | D399M/Y407A/ | | | | |
| | | K409V | | | | |
| 0% Combination 178 | T394W/F405A | E345R/Q347R/ | E356K/K392D/ | | | |
| | | K360D/T366V/ | D399K/K409D | | | |
| | | D399M/Y407A/ | | | | |
| | | K409V | | | | |
| 0% Combination 179 | T394W/F405A | Q347E/S354Y/ | E356K/K392D/ | | | |
| | | T366Y/Y407T | D399K/K439E | | | |
| 0% Combination 180 | T394W/F405A | Q347E/S354Y/ | K392D/D399K/ | | | |
| | | T366Y/Y407T | K409D | | | |
| 0% Combination 181 | T394W/F405A | Q347E/S354Y/ | E356K/K392D/ | | | |
| | | T366Y/Y407T | D399K/K409D | | | |
| 0% Combination 182 | T394W/F405A | Q347E/S354Y/ | E345R/K360E/ | | | |
| | | T366Y/Y407T | D399M/Y407A/ | | | |
| | | | K409V | | | |
| 0% Combination 183 | T394W/F405A | E356K/K392D/ | E345R/K360E/ | | | |
| | | D399K/K439E | D399M/Y407A/ | | | |
| | | | K409V | | | |
| 0% Combination 184 | T394W/F405A | E356K/K392D/ | E345R/K360E/ | | | |
| | | D399K/K409D | D399M/Y407A/ | | | |
| | | | K409V | | | |
| 0% Combination 185 | T366W/L368A/ | Q347R/K360E/ | E356K/D399K/ | | | |
| | Y407V | D399V/F405T/ | K409E/K439E | | | |
| | | K409W | | | | |
| 0% Combination 186 | T366W/L368A/ | Q347R/K360E/ | Y349C/E357C/ | | | |
| | Y407V | D399V/F405T/ | K392D/D399K | | | |
| | | K409W | | | | |
| 0% Combination 187 | T366W/L368A/ | Q347R/K360E/ | E356K/K392D/ | | | |
| | Y407V | D399V/F405T/ | D399K/K439E | | | |
| | | K409W | | | | |
| 0% Combination 188 | T366W/L368A/ | Q347R/K360E/ | K392D/D399K/ | | | |
| | Y407V | D399V/F405T/ | K409D | | | |
| | | K409W | | | | |
| 0% Combination 189 | T366W/L368A/ | Q347R/K360E/ | E356K/K392D/ | | | |
| | Y407V | D399V/F405T/ | D399K/K409D | | | |
| | | K409W | | | | |
| 0% Combination 190 | T366W/L368A/ | Q347R/K360E/ | T394F/F405A | | | |
| | Y407V | D399V/F405T/ | | | | |
| | | K409W | | | | |
| 0% Combination 191 | T366W/L368A/ | Q347R/K360E/ | K392L/T394W/ | | | |
| | Y407V | D399V/F405T/ | F405A/Y407V | | | |
| | | K409W | | | | |
| 0% Combination 192 | T366W/L368A/ | Q347R/K360E/ | T350V/L351Y/ | | | |
| | Y407V | D399V/F405T/ | T366L/K392L/ | | | |
| | | K409W | T394W/F405A/ | | | |
| | | | Y407V | | | |
| 0% Combination 193 | T366W/L368A/ | E356K/D399K/ | Y349C/E357C/ | | | |
| | Y407V | K409E/K439E | K392D/D399K | | | |
| 0% Combination 194 | T366W/L368A/ | E356K/D399K/ | E345R/Q347R/ | | | |
| | Y407V | K409E/K439E | K360D/T366V/ | | | |
| | | | D399M/Y407A/ | | | |
| | | | K409V | | | |
| 0% Combination 195 | T366W/L368A/ | E356K/D399K/ | E345R/K360E/ | | | |
| | Y407V | K409E/K439E | D399M/Y407A/ | | | |
| | | | K409V | | | |
| 0% Combination 196 | T366W/L368A/ | E356K/D399K/ | T394F/F405A | | | |
| | Y407V | K409E/K439E | | | | |
| 0% Combination 197 | T366W/L368A/ | E356K/D399K/ | K392L/T394W/ | | | |
| | Y407V | K409E/K439E | F405A/Y407V | | | |
| 0% Combination 198 | T366W/L368A/ | E356K/D399K/ | T350V/L351Y/ | | | |
| | Y407V | K409E/K439E | T366L/K392L/ | | | |
| | | | T394W/F405A/ | | | |
| | | | Y407V | | | |
| 0% Combination 199 | T366W/L368A/ | Y349C/E357C/ | E356K/K392D/ | | | |
| | Y407V | K392D/D399K | D399K/K439E | | | |
| 0% Combination 200 | T366W/L368A/ | Y349C/E357C/ | E356K/K392D/ | | | |
| | Y407V | K392D/D399K | D399K/K409D | | | |
| 0% Combination 201 | T366W/L368A/ | Y349C/E357C/ | E345R/K360E/ | | | |
| | Y407V | K392D/D399K | D399M/Y407A/ | | | |
| | | | K409V | | | |
| 0% Combination 202 | T366W/L368A/ | Y349C/E357C/ | T394F/F405A | | | |
| | Y407V | K392D/D399K | | | | |
| 0% Combination 203 | T366W/L368A/ | Y349C/E357C/ | K392L/T394W/ | | | |
| | Y407V | K392D/D399K | F405A/Y407V | | | |
| 0% Combination 204 | T366W/L368A/ | Y349C/E357C/ | T350V/L351Y/ | | | |
| | Y407V | K392D/D399K | T366L/K392L/ | | | |
| | | | T394W/F405A/ | | | |
| | | | Y407V | | | |
| 0% Combination 205 | T366W/L368A/ | E345R/Q347R/ | E356K/K392D/ | | | |
| | Y407V | K360D/T366V/ | D399K/K439E | | | |
| | | D399M/Y407A/ | | | | |
| | | K409V | | | | |
| 0% Combination 206 | T366W/L368A/ | E345R/Q347R/ | K392D/D399K/ | | | |
| | Y407V | K360D/T366V/ | K409D | | | |
| | | D399M/Y407A/ | | | | |
| | | K409V | | | | |
| 0% Combination 207 | T366W/L368A/ | E345R/Q347R/ | E356K/K392D/ | | | |
| | Y407V | K360D/T366V/ | D399K/K409D | | | |
| | | D399M/Y407A/ | | | | |
| | | K409V | | | | |
| 0% Combination 208 | T366W/L368A/ | E345R/Q347R/ | T394F/F405A | | | |
| | Y407V | K360D/T366V/ | | | | |
| | | D399M/Y407A/ | | | | |
| | | K409V | | | | |
| 0% Combination 209 | T366W/L368A/ | E345R/Q347R/ | K392L/T394W/ | | | |
| | Y407V | K360D/T366V/ | F405A/Y407V | | | |
| | | D399M/Y407A/ | | | | |
| | | K409V | | | | |
| 0% Combination 210 | T366W/L368A/ | E345R/Q347R/ | T350V/L351Y/ | | | |
| | Y407V | K360D/T366V/ | T366L/K392L/ | | | |
| | | D399M/Y407A/ | T394W/F405A/ | | | |
| | | K409V | Y407V | | | |
| 0% Combination 211 | T366W/L368A/ | E356K/K392D/ | E345R/K360E/ | | | |
| | Y407V | D399K/K439E | D399M/Y407A/ | | | |
| | | | K409V | | | |
| 0% Combination 212 | T366W/L368A/ | E356K/K392D/ | T394F/F405A | | | |
| | Y407V | D399K/K439E | | | | |
| 0% Combination 213 | T366W/L368A/ | E356K/K392D/ | K392L/T394W/ | | | |
| | Y407V | D399K/K439E | F405A/Y407V | | | |
| 0% Combination 214 | T366W/L368A/ | E356K/K392D/ | T350V/L351Y/ | | | |
| | Y407V | D399K/K439E | T366L/K392L/ | | | |
| | | | T394W/F405A/ | | | |
| | | | Y407V | | | |
| 0% Combination 215 | T366W/L368A/ | K392D/D399K/ | T394F/F405A | | | |
| | Y407V | K409D | | | | |
| 0% Combination 216 | T366W/L368A/ | K392D/D399K/ | K392L/T394W/ | | | |
| | Y407V | K409D | F405A/Y407V | | | |
| 0% Combination 217 | T366W/L368A/ | K392D/D399K/ | T350V/L351Y/ | | | |
| | Y407V | K409D | T366L/K392L/ | | | |
| | | | T394W/F405A/ | | | |
| | | | Y407V | | | |
| 0% Combination 218 | T366W/L368A/ | E356K/K392D/ | E345R/K360E/ | | | |
| | Y407V | D399K/K409D | D399M/Y407A/ | | | |
| | | | K409V | | | |
| 0% Combination 219 | T366W/L368A/ | E356K/K392D/ | T394F/F405A | | | |
| | Y407V | D399K/K409D | | | | |
| 0% Combination 220 | T366W/L368A/ | E356K/K392D/ | K392L/T394W/ | | | |
| | Y407V | D399K/K409D | F405A/Y407V | | | |
| 0% Combination 221 | T366W/L368A/ | E356K/K392D/ | T350V/L351Y/ | | | |
| | Y407V | D399K/K409D | T366L/K392L/ | | | |
| | | | T394W/F405A/ | | | |
| | | | Y407V | | | |
| 0% Combination 222 | T366W/L368A/ | E345R/K360E/ | T394F/F405A | | | |
| | Y407V | D399M/Y407A/ | | | | |
| | | K409V | | | | |
| 0% Combination 223 | T366W/L368A/ | E345R/K360E/ | K392L/T394W/ | | | |
| | Y407V | D399M/Y407A/ | F405A/Y407V | | | |
| | | K409V | | | | |
| 0% Combination 224 | T366W/L368A/ | E345R/K360E/ | T350V/L351Y/ | | | |
| | Y407V | D399M/Y407A/ | T366L/K392L/ | | | |
| | | K409V | T394W/F405A/ | | | |
| | | | Y407V | | | |
| 0% Combination 225 | Q347R/K360E/ | E356K/D399K/ | Y349C/E357C/ | | | |
| | D399V/F405T/ | K409E/K439E | K392D/D399K | | | |
| | K409W | | | | | |
| 0% Combination 226 | Q347R/K360E/ | E356K/D399K/ | Q347E/S354Y/ | | | |
| | D399V/F405T/ | K409E/K439E | T366Y/Y407T | | | |
| | K409W | | | | | |
| 0% Combination 227 | Q347R/K360E/ | E356K/D399K/ | T394F/F405A | | | |
| | D399V/F405T/ | K409E/K439E | | | | |
| | K409W | | | | | |
| 0% Combination 228 | Q347R/K360E/ | E356K/D399K/ | K392L/T394W/ | | | |
| | D399V/F405T/ | K409E/K439E | F405A/Y407V | | | |
| | K409W | | | | | |
| 0% Combination 229 | Q347R/K360E/ | E356K/D399K/ | T350V/L351Y/ | | | |
| | D399V/F405T/ | K409E/K439E | T366L/K392L/ | | | |
| | K409W | | T394W/F405A/ | | | |
| | | | Y407V | | | |
| 0% Combination 230 | Q347R/K360E/ | Y349C/E357C/ | E356K/K392D/ | | | |
| | D399V/F405T/ | K392D/D399K | D399K/K439E | | | |
| | K409W | | | | | |
| 0% Combination 231 | Q347R/K360E/ | Y349C/E357C/ | E356K/K392D/ | | | |
| | D399V/F405T/ | K392D/D399K | D399K/K409D | | | |
| | K409W | | | | | |
| 0% Combination 232 | Q347R/K360E/ | Y349C/E357C/ | T394F/F405A | | | |
| | D399V/F405T/ | K392D/D399K | | | | |
| | K409W | | | | | |
| 0% Combination 233 | Q347R/K360E/ | Y349C/E357C/ | K392L/T394W/ | | | |
| | D399V/F405T/ | K392D/D399K | F405A/Y407V | | | |
| | K409W | | | | | |
| 0% Combination 234 | Q347R/K360E/ | Y349C/E357C/ | T350V/L351Y/ | | | |
| | D399V/F405T/ | K392D/D399K | T366L/K392L/ | | | |
| | K409W | | T394W/F405A/ | | | |
| | | | Y407V | | | |
| 0% Combination 235 | Q347R/K360E/ | Q347E/S354Y/ | E356K/K392D/ | | | |
| | D399V/F405T/ | T366Y/Y407T | D399K/K439E | | | |
| | K409W | | | | | |
| 0% Combination 236 | Q347R/K360E/ | Q347E/S354Y/ | K392D/D399K/ | | | |
| | D399V/F405T/ | T366Y/Y407T | K409D | | | |
| | K409W | | | | | |
| 0% Combination 237 | Q347R/K360E/ | Q347E/S354Y/ | E356K/K392D/ | | | |
| | D399V/F405T/ | T366Y/Y407T | D399K/K409D | | | |
| | K409W | | | | | |
| 0% Combination 238 | Q347R/K360E/ | Q347E/S354Y/ | T394F/F405A | | | |
| | D399V/F405T/ | T366Y/Y407T | | | | |
| | K409W | | | | | |
| 0% Combination 239 | Q347R/K360E/ | Q347E/S354Y/ | K392L/T394W/ | | | |
| | D399V/F405T/ | T366Y/Y407T | F405A/Y407V | | | |
| | K409W | | | | | |
| 0% Combination 240 | Q347R/K360E/ | Q347E/S354Y/ | T350V/L351Y/ | | | |
| | D399V/F405T/ | T366Y/Y407T | T366L/K392L/ | | | |
| | K409W | | T394W/F405A/ | | | |
| | | | Y407V | | | |
| 0% Combination 241 | Q347R/K360E/ | E356K/K392D/ | T394F/F405A | | | |
| | D399V/F405T/ | D399K/K439E | | | | |
| | K409W | | | | | |
| 0% Combination 242 | Q347R/K360E/ | E356K/K392D/ | K392L/T394W/ | | | |
| | D399V/F405T/ | D399K/K439E | F405A/Y407V | | | |
| | K409W | | | | | |
| 0% Combination 243 | Q347R/K360E/ | E356K/K392D/ | T350V/L351Y/ | | | |
| | D399V/F405T/ | D399K/K439E | T366L/K392L/ | | | |
| | K409W | | T394W/F405A/ | | | |
| | | | Y407V | | | |
| 0% Combination 244 | Q347R/K360E/ | K392D/D399K/ | T394F/F405A | | | |
| | D399V/F405T/ | K409D | | | | |
| | K409W | | | | | |
| 0% Combination 245 | Q347R/K360E/ | K392D/D399K/ | K392L/T394W/ | | | |
| | D399V/F405T/ | K409D | F405A/Y407V | | | |
| | K409W | | | | | |
| 0% Combination 246 | Q347R/K360E/ | K392D/D399K/ | T350V/L351Y/ | | | |
| | D399V/F405T/ | K409D | T366L/K392L/ | | | |
| | K409W | | T394W/F405A/ | | | |
| | | | Y407V | | | |
| 0% Combination 247 | Q347R/K360E/ | E356K/K392D/ | T394F/F405A | | | |
| | D399V/F405T/ | D399K/K409D | | | | |
| | K409W | | | | | |
| 0% Combination 248 | Q347R/K360E/ | E356K/K392D/ | K392L/T394W/ | | | |
| | D399V/F405T/ | D399K/K409D | F405A/Y407V | | | |
| | K409W | | | | | |
| 0% Combination 249 | Q347R/K360E/ | E356K/K392D/ | T350V/L351Y/ | | | |
| | D399V/F405T/ | D399K/K409D | T366L/K392L/ | | | |
| | K409W | | T394W/F405A/ | | | |
| | | | Y407V | | | |
| 0% Combination 250 | E356K/D399K/ | Y349C/E357C/ | E345R/K360E/ | | | |
| | K409E/K439E | K392D/D399K | D399M/Y407A/ | | | |
| | | | K409V | | | |
| 0% Combination 251 | E356K/D399K/ | Y349C/E357C/ | T394F/F405A | | | |
| | K409E/K439E | K392D/D399K | | | | |
| 0% Combination 252 | E356K/D399K/ | Y349C/E357C/ | K392L/T394W/ | | | |
| | K409E/K439E | K392D/D399K | F405A/Y407V | | | |
| 0% Combination 253 | E356K/D399K/ | Y349C/E357C/ | T350V/L351Y/ | | | |
| | K409E/K439E | K392D/D399K | T366L/K392L/ | | | |
| | | | T394W/F405A/ | | | |
| | | | Y407V | | | |
| 0% Combination 254 | E356K/D399K/ | E345R/Q347R/ | T394F/F405A | | | |
| | K409E/K439E | K360D/T366V/ | | | | |
| | | D399M/Y407A/ | | | | |
| | | K409V | | | | |
| 0% Combination 255 | E356K/D399K/ | E345R/Q347R/ | K392L/T394W/ | | | |
| | K409E/K439E | K360D/T366V/ | F405A/Y407V | | | |
| | | D399M/Y407A/ | | | | |
| | | K409V | | | | |
| 0% Combination 256 | E356K/D399K/ | E345R/Q347R/ | T350V/L351Y/ | | | |
| | K409E/K439E | K360D/T366V/ | T366L/K392L/ | | | |
| | | D399M/Y407A/ | T394W/F405A/ | | | |
| | | K409V | Y407V | | | |
| 0% Combination 257 | E356K/D399K/ | Q347E/S354Y/ | E345R/K360E/ | | | |
| | K409E/K439E | T366Y/Y407T | D399M/Y407A/ | | | |
| | | | K409V | | | |
| 0% Combination 258 | E356K/D399K/ | Q347E/S354Y/ | T394F/F405A | | | |
| | K409E/K439E | T366Y/Y407T | | | | |
| 0% Combination 259 | E356K/D399K/ | Q347E/S354Y/ | K392L/T394W/ | | | |
| | K409E/K439E | T366Y/Y407T | F405A/Y407V | | | |
| 0% Combination 260 | E356K/D399K/ | Q347E/S354Y/ | T350V/L351Y/ | | | |
| | K409E/K439E | T366Y/Y407T | T366L/K392L/ | | | |
| | | | T394W/F405A/ | | | |
| | | | Y407V | | | |
| 0% Combination 261 | E356K/D399K/ | E345R/K360E/ | T394F/F405A | | | |
| | K409E/K439E | D399M/Y407A/ | | | | |
| | | K409V | | | | |
| 0% Combination 262 | E356K/D399K/ | E345R/K360E/ | K392L/T394W/ | | | |
| | K409E/K439E | D399M/Y407A/ | F405A/Y407V | | | |
| | | K409V | | | | |
| 0% Combination 263 | E356K/D399K/ | E345R/K360E/ | T350V/L351Y/ | | | |
| | K409E/K439E | D399M/Y407A/ | T366L/K392L/ | | | |
| | | K409V | T394W/F405A/ | | | |
| | | | Y407V | | | |
| 0% Combination 264 | Y349C/E357C/ | E356K/K392D/ | E345R/K360E/ | | | |
| | K392D/D399K | D399K/K439E | D399M/Y407A/ | | | |
| | | | K409V | | | |
| 0% Combination 265 | Y349C/E357C/ | E356K/K392D/ | T394F/F405A | | | |
| | K392D/D399K | D399K/K439E | | | | |
| 0% Combination 266 | Y349C/E357C/ | E356K/K392D/ | K392L/T394W/ | | | |
| | K392D/D399K | D399K/K439E | F405A/Y407V | | | |
| 0% Combination 267 | Y349C/E357C/ | E356K/K392D/ | T350V/L351Y/ | | | |
| | K392D/D399K | D399K/K439E | T366L/K392L/ | | | |
| | | | T394W/F405A/ | | | |
| | | | Y407V | | | |
| 0% Combination 268 | Y349C/E357C/ | E356K/K392D/ | E345R/K360E/ | | | |
| | K392D/D399K | D399K/K409D | D399M/Y407A/ | | | |
| | | | K409V | | | |
| 0% Combination 269 | Y349C/E357C/ | E356K/K392D/ | T394F/F405A | | | |
| | K392D/D399K | D399K/K409D | | | | |
| 0% Combination 270 | Y349C/E357C/ | E356K/K392D/ | K392L/T394W/ | | | |
| | K392D/D399K | D399K/K409D | F405A/Y407V | | | |
| 0% Combination 271 | Y349C/E357C/ | E356K/K392D/ | T350V/L351Y/ | | | |
| | K392D/D399K | D399K/K409D | T366L/K392L/ | | | |
| | | | T394W/F405A/ | | | |
| | | | Y407V | | | |
| 0% Combination 272 | Y349C/E357C/ | E345R/K360E/ | T394F/F405A | | | |
| | K392D/D399K | D399M/Y407A/ | | | | |
| | | K409V | | | | |
| 0% Combination 273 | Y349C/E357C/ | E345R/K360E/ | K392L/T394W/ | | | |
| | K392D/D399K | D399M/Y407A/ | F405A/Y407V | | | |
| | | K409V | | | | |
| 0% Combination 274 | Y349C/E357C/ | E345R/K360E/ | T350V/L351Y/ | | | |
| | K392D/D399K | D399M/Y407A/ | T366L/K392L/ | | | |
| | | K409V | T394W/F405A/ | | | |
| | | | Y407V | | | |
| 0% Combination 275 | E345R/Q347R/ | E356K/K392D/ | T394F/F405A | | | |
| | K360D/T366V/ | D399K/K439E | | | | |
| | D399M/Y407A/ | | | | | |
| | K409V | | | | | |
| 0% Combination 276 | E345R/Q347R/ | E356K/K392D/ | K392L/T394W/ | | | |
| | K360D/T366V/ | D399K/K439E | F405A/Y407V | | | |
| | D399M/Y407A/ | | | | | |
| | K409V | | | | | |
| 0% Combination 277 | E345R/Q347R/ | E356K/K392D/ | T350V/L351Y/ | | | |
| | K360D/T366V/ | D399K/K439E | T366L/K392L/ | | | |
| | D399M/Y407A/ | | T394W/F405A/ | | | |
| | K409V | | Y407V | | | |
| 0% Combination 278 | E345R/Q347R/ | K392D/D399K/ | T394F/F405A | | | |
| | K360D/T366V/ | K409D | | | | |
| | D399M/Y407A/ | | | | | |
| | K409V | | | | | |
| 0% Combination 279 | E345R/Q347R/ | K392D/D399K/ | K392L/T394W/ | | | |
| | K360D/T366V/ | K409D | F405A/Y407V | | | |
| | D399M/Y407A/ | | | | | |
| | K409V | | | | | |
| 0% Combination 280 | E345R/Q347R/ | K392D/D399K/ | T350V/L351Y/ | | | |
| | K360D/T366V/ | K409D | T366L/K392L/ | | | |
| | D399M/Y407A/ | | T394W/F405A/ | | | |
| | K409V | | Y407V | | | |
| 0% Combination 281 | E345R/Q347R/ | E356K/K392D/ | T394F/F405A | | | |
| | K360D/T366V/ | D399K/K409D | | | | |
| | D399M/Y407A/ | | | | | |
| | K409V | | | | | |
| 0% Combination 282 | E345R/Q347R/ | E356K/K392D/ | K392L/T394W/ | | | |
| | K360D/T366V/ | D399K/K409D | F405A/Y407V | | | |
| | D399M/Y407A/ | | | | | |
| | K409V | | | | | |
| 0% Combination 283 | E345R/Q347R/ | E356K/K392D/ | T350V/L351Y/ | | | |
| | K360D/T366V/ | D399K/K409D | T366L/K392L/ | | | |
| | D399M/Y407A/ | | T394W/F405A/ | | | |
| | K409V | | Y407V | | | |
| 0% Combination 284 | Q347E/S354Y/ | E356K/K392D/ | E345R/K360E/ | | | |
| | T366Y/Y407T | D399K/K439E | D399M/Y407A/ | | | |
| | | | K409V | | | |
| 0% Combination 285 | Q347E/S354Y/ | E356K/K392D/ | T394F/F405A | | | |
| | T366Y/Y407T | D399K/K439E | | | | |
| 0% Combination 286 | Q347E/S354Y/ | E356K/K392D/ | K392L/T394W/ | | | |
| | T366Y/Y407T | D399K/K439E | F405A/Y407V | | | |
| 0% Combination 287 | Q347E/S354Y/ | E356K/K392D/ | T350V/L351Y/ | | | |
| | T366Y/Y407T | D399K/K439E | T366L/K392L/ | | | |
| | | | T394W/F405A/ | | | |
| | | | Y407V | | | |
| 0% Combination 288 | Q347E/S354Y/ | K392D/D399K/ | T394F/F405A | | | |
| | T366Y/Y407T | K409D | | | | |
| 0% Combination 289 | Q347E/S354Y/ | K392D/D399K/ | K392L/T394W/ | | | |
| | T366Y/Y407T | K409D | F405A/Y407V | | | |
| 0% Combination 290 | Q347E/S354Y/ | K392D/D399K/ | T350V/L351Y/ | | | |
| | T366Y/Y407T | K409D | T366L/K392L/ | | | |
| | | | T394W/F405A/ | | | |
| | | | Y407V | | | |
| 0% Combination 291 | Q347E/S354Y/ | E356K/K392D/ | E345R/K360E/ | | | |
| | T366Y/Y407T | D399K/K409D | D399M/Y407A/ | | | |
| | | | K409V | | | |
| 0% Combination 292 | Q347E/S354Y/ | E356K/K392D/ | T394F/F405A | | | |
| | T366Y/Y407T | D399K/K409D | | | | |
| 0% Combination 293 | Q347E/S354Y/ | E356K/K392D/ | K392L/T394W/ | | | |
| | T366Y/Y407T | D399K/K409D | F405A/Y407V | | | |
| 0% Combination 294 | Q347E/S354Y/ | E356K/K392D/ | T350V/L351Y/ | | | |
| | T366Y/Y407T | D399K/K409D | T366L/K392L/ | | | |
| | | | T394W/F405A/ | | | |
| | | | Y407V | | | |
| 0% Combination 295 | Q347E/S354Y/ | E345R/K360E/ | T394F/F405A | | | |
| | T366Y/Y407T | D399M/Y407A/ | | | | |
| | | K409V | | | | |
| 0% Combination 296 | Q347E/S354Y/ | E345R/K360E/ | K392L/T394W/ | | | |
| | T366Y/Y407T | D399M/Y407A/ | F405A/Y407V | | | |
| | | K409V | | | | |
| 0% Combination 297 | Q347E/S354Y/ | E345R/K360E/ | T350V/L351Y/ | | | |
| | T366Y/Y407T | D399M/Y407A/ | T366L/K392L/ | | | |
| | | K409V | T394W/F405A/ | | | |
| | | | Y407V | | | |
| 0% Combination 298 | E356K/K392D/ | E345R/K360E/ | T394F/F405A | | | |
| | D399K/K439E | D399M/Y407A/ | | | | |
| | | K409V | | | | |
| 0% Combination 299 | E356K/K392D/ | E345R/K360E/ | K392L/T394W/ | | | |
| | D399K/K439E | D399M/Y407A/ | F405A/Y407V | | | |
| | | K409V | | | | |
| 0% Combination 300 | E356K/K392D/ | E345R/K360E/ | T350V/L351Y/ | | | |
| | D399K/K439E | D399M/Y407A/ | T366L/K392L/ | | | |
| | | K409V | T394W/F405A/ | | | |
| | | | Y407V | | | |
| 0% Combination 301 | E356K/K392D/ | E345R/K360E/ | T394F/F405A | | | |
| | D399K/K409D | D399M/Y407A/ | | | | |
| | | K409V | | | | |
| 0% Combination 302 | E356K/K392D/ | E345R/K360E/ | K392L/T394W/ | | | |
| | D399K/K409D | D399M/Y407A/ | F405A/Y407V | | | |
| | | K409V | | | | |
| 0% Combination 303 | E356K/K392D/ | E345R/K360E/ | T350V/L351Y/ | | | |
| | D399K/K409D | D399M/Y407A/ | T366L/K392L/ | | | |
| | | K409V | T394W/F405A/ | | | |
| | | | Y407V | | | |
| 0% Combination 304 | WT | T394W/F405A | T366W/L368A/ | Q347R/K360E/ | | |
| | | | Y407V | D399V/F405T/ | | |
| | | | | K409W | | |
| 0% Combination 305 | WT | T394W/F405A | T366W/L368A/ | E356K/D399K/ | | |
| | | | Y407V | K409E/K439E | | |
| 0% Combination 306 | WT | T394W/F405A | T366W/L368A/ | Y349C/E357C/ | | |
| | | | Y407V | K392D/D399K | | |
| 0% Combination 307 | WT | T394W/F405A | T366W/L368A/ | E345R/Q347R/ | | |
| | | | Y407V | K360D/T366V/ | | |
| | | | | D399M/Y407A/ | | |
| | | | | K409V | | |
| 0% Combination 308 | WT | T394W/F405A | T366W/L368A/ | E356K/K392D/ | | |
| | | | Y407V | D399K/K439E | | |
| 0% Combination 309 | WT | T394W/F405A | T366W/L368A/ | K392D/D399K/ | | |
| | | | Y407V | K409D | | |
| 0% Combination 310 | WT | T394W/F405A | T366W/L368A/ | E356K/K392D/ | | |
| | | | Y407V | D399K/K409D | | |
| 0% Combination 311 | WT | T394W/F405A | T366W/L368A/ | E345R/K360E/ | | |
| | | | Y407V | D399M/Y407A/ | | |
| | | | | K409V | | |
| 0% Combination 312 | WT | T394W/F405A | Q347R/K360E/ | E356K/D399K/ | | |
| | | | D399V/F405T/ | K409E/K439E | | |
| | | | K409W | | | |
| 0% Combination 313 | WT | T394W/F405A | Q347R/K360E/ | Y349C/E357C/ | | |
| | | | D399V/F405T/ | K392D/D399K | | |
| | | | K409W | | | |
| 0% Combination 314 | WT | T394W/F405A | Q347R/K360E/ | Q347E/S354Y/ | | |
| | | | D399V/F405T/ | T366Y/Y407T | | |
| | | | K409W | | | |
| 0% Combination 315 | WT | T394W/F405A | Q347R/K360E/ | E356K/K392D/ | | |
| | | | D399V/F405T/ | D399K/K439E | | |
| | | | K409W | | | |
| 0% Combination 316 | WT | T394W/F405A | Q347R/K360E/ | K392D/D399K/ | | |
| | | | D399V/F405T/ | K409D | | |
| | | | K409W | | | |
| 0% Combination 317 | WT | T394W/F405A | Q347R/K360E/ | E356K/K392D/ | | |
| | | | D399V/F405T/ | D399K/K409D | | |
| | | | K409W | | | |
| 0% Combination 318 | WT | T394W/F405A | E356K/D399K/ | Y349C/E357C/ | | |
| | | | K409E/K439E | K392D/D399K | | |
| 0% Combination 319 | WT | T394W/F405A | E356K/D399K/ | E345R/Q347R/ | | |
| | | | K409E/K439E | K360D/T366V/ | | |
| | | | | D399M/Y407A/ | | |
| | | | | K409V | | |
| 0% Combination 320 | WT | T394W/F405A | E356K/D399K/ | Q347E/S354Y/ | | |
| | | | K409E/K439E | T366Y/Y407T | | |
| 0% Combination 321 | WT | T394W/F405A | E356K/D399K/ | E345R/K360E/ | | |
| | | | K409E/K439E | D399M/Y407A/ | | |
| | | | | K409V | | |
| 0% Combination 322 | WT | T394W/F405A | Y349C/E357C/ | E356K/K392D/ | | |
| | | | K392D/D399K | D399K/K439E | | |
| 0% Combination 323 | WT | T394W/F405A | Y349C/E357C/ | E356K/K392D/ | | |
| | | | K392D/D399K | D399K/K409D | | |
| 0% Combination 324 | WT | T394W/F405A | Y349C/E357C/ | E345R/K360E/ | | |
| | | | K392D/D399K | D399M/Y407A/ | | |
| | | | | K409V | | |
| 0% Combination 325 | WT | T394W/F405A | E345R/Q347R/ | E356K/K392D/ | | |
| | | | K360D/T366V/ | D399K/K439E | | |
| | | | D399M/Y407A/ | | | |
| | | | K409V | | | |
| 0% Combination 326 | WT | T394W/F405A | E345R/Q347R/ | K392D/D399K/ | | |
| | | | K360D/T366V/ | K409D | | |
| | | | D399M/Y407A/ | | | |
| | | | K409V | | | |
| 0% Combination 327 | WT | T394W/F405A | E345R/Q347R/ | E356K/K392D/ | | |
| | | | K360D/T366V/ | D399K/K409D | | |
| | | | D399M/Y407A/ | | | |
| | | | K409V | | | |
| 0% Combination 328 | WT | T394W/F405A | Q347E/S354Y/ | E356K/K392D/ | | |
| | | | T366Y/Y407T | D399K/K439E | | |
| 0% Combination 329 | WT | T394W/F405A | Q347E/S354Y/ | K392D/D399K/ | | |
| | | | T366Y/Y407T | K409D | | |
| 0% Combination 330 | WT | T394W/F405A | Q347E/S354Y/ | E356K/K392D/ | | |
| | | | T366Y/Y407T | D399K/K409D | | |
| 0% Combination 331 | WT | T394W/F405A | Q347E/S354Y/ | E345R/K360E/ | | |
| | | | T366Y/Y407T | D399M/Y407A/ | | |
| | | | | K409V | | |
| 0% Combination 332 | WT | T394W/F405A | E356K/K392D/ | E345R/K360E/ | | |
| | | | D399K/K439E | D399M/Y407A/ | | |
| | | | | K409V | | |
| 0% Combination 333 | WT | T394W/F405A | E356K/K392D/ | E345R/K360E/ | | |
| | | | D399K/K409D | D399M/Y407A/ | | |
| | | | | K409V | | |
| 0% Combination 334 | WT | T366W/L368A/ | Q347R/K360E/ | E356K/D399K/ | | |
| | | Y407V | D399V/F405T/ | K409E/K439E | | |
| | | | K409W | | | |
| 0% Combination 335 | WT | T366W/L368A/ | Q347R/K360E/ | Y349C/E357C/ | | |
| | | Y407V | D399V/F405T/ | K392D/D399K | | |
| | | | K409W | | | |
| 0% Combination 336 | WT | T366W/L368A/ | Q347R/K360E/ | E356K/K392D/ | | |
| | | Y407V | D399V/F405T/ | D399K/K439E | | |
| | | | K409W | | | |
| 0% Combination 337 | WT | T366W/L368A/ | Q347R/K360E/ | K392D/D399K/ | | |
| | | Y407V | D399V/F405T/ | K409D | | |
| | | | K409W | | | |
| 0% Combination 338 | WT | T366W/L368A/ | Q347R/K360E/ | E356K/K392D/ | | |
| | | Y407V | D399V/F405T/ | D399K/K409D | | |
| | | | K409W | | | |
| 0% Combination 339 | WT | T366W/L368A/ | Q347R/K360E/ | T394F/F405A | | |
| | | Y407V | D399V/F405T/ | | | |
| | | | K409W | | | |
| 0% Combination 340 | WT | T366W/L368A/ | Q347R/K360E/ | K392L/T394W/ | | |
| | | Y407V | D399V/F405T/ | F405A/Y407V | | |
| | | | K409W | | | |
| 0% Combination 341 | WT | T366W/L368A/ | Q347R/K360E/ | T350V/L351Y/ | | |
| | | Y407V | D399V/F405T/ | T366L/K392L/ | | |
| | | | K409W | T394W/F405A/ | | |
| | | | | Y407V | | |
| 0% Combination 342 | WT | T366W/L368A/ | E356K/D399K/ | Y349C/E357C/ | | |
| | | Y407V | K409E/K439E | K392D/D399K | | |
| 0% Combination 343 | WT | T366W/L368A/ | E356K/D399K/ | E345R/Q347R/ | | |
| | | Y407V | K409E/K439E | K360D/T366V/ | | |
| | | | | D399M/Y407A/ | | |
| | | | | K409V | | |
| 0% Combination 344 | WT | T366W/L368A/ | E356K/D399K/ | E345R/K360E/ | | |
| | | Y407V | K409E/K439E | D399M/Y407A/ | | |
| | | | | K409V | | |
| 0% Combination 345 | WT | T366W/L368A/ | E356K/D399K/ | T394F/F405A | | |
| | | Y407V | K409E/K439E | | | |
| 0% Combination 346 | WT | T366W/L368A/ | E356K/D399K/ | K392L/T394W/ | | |
| | | Y407V | K409E/K439E | F405A/Y407V | | |
| 0% Combination 347 | WT | T366W/L368A/ | E356K/D399K/ | T350V/L351Y/ | | |
| | | Y407V | K409E/K439E | T366L/K392L/ | | |
| | | | | T394W/F405A/ | | |
| | | | | Y407V | | |
| 0% Combination 348 | WT | T366W/L368A/ | Y349C/E357C/ | E356K/K392D/ | | |
| | | Y407V | K392D/D399K | D399K/K439E | | |
| 0% Combination 349 | WT | T366W/L368A/ | Y349C/E357C/ | E356K/K392D/ | | |
| | | Y407V | K392D/D399K | D399K/K409D | | |
| 0% Combination 350 | WT | T366W/L368A/ | Y349C/E357C/ | E345R/K360E/ | | |
| | | Y407V | K392D/D399K | D399M/Y407A/ | | |
| | | | | K409V | | |
| 0% Combination 351 | WT | T366W/L368A/ | Y349C/E357C/ | T394F/F405A | | |
| | | Y407V | K392D/D399K | | | |
| 0% Combination 352 | WT | T366W/L368A/ | Y349C/E357C/ | K392L/T394W/ | | |
| | | Y407V | K392D/D399K | F405A/Y407V | | |
| 0% Combination 353 | WT | T366W/L368A/ | Y349C/E357C/ | T350V/L351Y/ | | |
| | | Y407V | K392D/D399K | T366L/K392L/ | | |
| | | | | T394W/F405A/ | | |
| | | | | Y407V | | |
| 0% Combination 354 | WT | T366W/L368A/ | E345R/Q347R/ | E356K/K392D/ | | |
| | | Y407V | K360D/T366V/ | D399K/K439E | | |
| | | | D399M/Y407A/ | | | |
| | | | K409V | | | |
| 0% Combination 355 | WT | T366W/L368A/ | E345R/Q347R/ | K392D/D399K/ | | |
| | | Y407V | K360D/T366V/ | K409D | | |
| | | | D399M/Y407A/ | | | |
| | | | K409V | | | |
| 0% Combination 356 | WT | T366W/L368A/ | E345R/Q347R/ | E356K/K392D/ | | |
| | | Y407V | K360D/T366V/ | D399K/K409D | | |
| | | | D399M/Y407A/ | | | |
| | | | K409V | | | |
| 0% Combination 357 | WT | T366W/L368A/ | E345R/Q347R/ | T394F/F405A | | |
| | | Y407V | K360D/T366V/ | | | |
| | | | D399M/Y407A/ | | | |
| | | | K409V | | | |
| 0% Combination 358 | WT | T366W/L368A/ | E345R/Q347R/ | K392L/T394W/ | | |
| | | Y407V | K360D/T366V/ | F405A/Y407V | | |
| | | | D399M/Y407A/ | | | |
| | | | K409V | | | |
| 0% Combination 359 | WT | T366W/L368A/ | E345R/Q347R/ | T350V/L351Y/ | | |
| | | Y407V | K360D/T366V/ | T366L/K392L/ | | |
| | | | D399M/Y407A/ | T394W/F405A/ | | |
| | | | K409V | Y407V | | |
| 0% Combination 360 | WT | T366W/L368A/ | E356K/K392D/ | E345R/K360E/ | | |
| | | Y407V | D399K/K439E | D399M/Y407A/ | | |
| | | | | K409V | | |
| 0% Combination 361 | WT | T366W/L368A/ | E356K/K392D/ | T394F/F405A | | |
| | | Y407V | D399K/K439E | | | |
| 0% Combination 362 | WT | T366W/L368A/ | E356K/K392D/ | K392L/T394W/ | | |
| | | Y407V | D399K/K439E | F405A/Y407V | | |
| 0% Combination 363 | WT | T366W/L368A/ | E356K/K392D/ | T350V/L351Y/ | | |
| | | Y407V | D399K/K439E | T366L/K392L/ | | |
| | | | | T394W/F405A/ | | |
| | | | | Y407V | | |
| 0% Combination 364 | WT | T366W/L368A/ | K392D/D399K/ | T394F/F405A | | |
| | | Y407V | K409D | | | |
| 0% Combination 365 | WT | T366W/L368A/ | K392D/D399K/ | K392L/T394W/ | | |
| | | Y407V | K409D | F405A/Y407V | | |
| 0% Combination 366 | WT | T366W/L368A/ | K392D/D399K/ | T350V/L351Y/ | | |
| | | Y407V | K409D | T366L/K392L/ | | |
| | | | | T394W/F405A/ | | |
| | | | | Y407V | | |
| 0% Combination 367 | WT | T366W/L368A/ | E356K/K392D/ | E345R/K360E/ | | |
| | | Y407V | D399K/K409D | D399M/Y407A/ | | |
| | | | | K409V | | |
| 0% Combination 368 | WT | T366W/L368A/ | E356K/K392D/ | T394F/F405A | | |
| | | Y407V | D399K/K409D | | | |
| 0% Combination 369 | WT | T366W/L368A/ | E356K/K392D/ | K392L/T394W/ | | |
| | | Y407V | D399K/K409D | F405A/Y407V | | |
| 0% Combination 370 | WT | T366W/L368A/ | E356K/K392D/ | T350V/L351Y/ | | |
| | | Y407V | D399K/K409D | T366L/K392L/ | | |
| | | | | T394W/F405A/ | | |
| | | | | Y407V | | |
| 0% Combination 371 | WT | T366W/L368A/ | E345R/K360E/ | T394F/F405A | | |
| | | Y407V | D399M/Y407A/ | | | |
| | | | K409V | | | |
| 0% Combination 372 | WT | T366W/L368A/ | E345R/K360E/ | K392L/T394W/ | | |
| | | Y407V | D399M/Y407A/ | F405A/Y407V | | |
| | | | K409V | | | |
| 0% Combination 373 | WT | T366W/L368A/ | E345R/K360E/ | T350V/L351Y/ | | |
| | | Y407V | D399M/Y407A/ | T366L/K392L/ | | |
| | | | K409V | T394W/F405A/ | | |
| | | | | Y407V | | |
| 0% Combination 374 | WT | Q347R/K360E/ | E356K/D399K/ | Y349C/E357C/ | | |
| | | D399V/F405T/ | K409E/K439E | K392D/D399K | | |
| | | K409W | | | | |
| 0% Combination 375 | WT | Q347R/K360E/ | E356K/D399K/ | Q347E/S354Y/ | | |
| | | D399V/F405T/ | K409E/K439E | T366Y/Y407T | | |
| | | K409W | | | | |
| 0% Combination 376 | WT | Q347R/K360E/ | E356K/D399K/ | T394F/F405A | | |
| | | D399V/F405T/ | K409E/K439E | | | |
| | | K409W | | | | |
| 0% Combination 377 | WT | Q347R/K360E/ | E356K/D399K/ | K392L/T394W/ | | |
| | | D399V/F405T/ | K409E/K439E | F405A/Y407V | | |
| | | K409W | | | | |
| 0% Combination 378 | WT | Q347R/K360E/ | E356K/D399K/ | T350V/L351Y/ | | |
| | | D399V/F405T/ | K409E/K439E | T366L/K392L/ | | |
| | | K409W | | T394W/F405A/ | | |
| | | | | Y407V | | |
| 0% Combination 379 | WT | Q347R/K360E/ | Y349C/E357C/ | E356K/K392D/ | | |
| | | D399V/F405T/ | K392D/D399K | D399K/K439E | | |
| | | K409W | | | | |
| 0% Combination 380 | WT | Q347R/K360E/ | Y349C/E357C/ | E356K/K392D/ | | |
| | | D399V/F405T/ | K392D/D399K | D399K/K409D | | |
| | | K409W | | | | |
| 0% Combination 381 | WT | Q347R/K360E/ | Y349C/E357C/ | T394F/F405A | | |
| | | D399V/F405T/ | K392D/D399K | | | |
| | | K409W | | | | |
| 0% Combination 382 | WT | Q347R/K360E/ | Y349C/E357C/ | K392L/T394W/ | | |
| | | D399V/F405T/ | K392D/D399K | F405A/Y407V | | |
| | | K409W | | | | |
| 0% Combination 383 | WT | Q347R/K360E/ | Y349C/E357C/ | T350V/L351Y/ | | |
| | | D399V/F405T/ | K392D/D399K | T366L/K392L/ | | |
| | | K409W | | T394W/F405A/ | | |
| | | | | Y407V | | |
| 0% Combination 384 | WT | Q347R/K360E/ | Q347E/S354Y/ | E356K/K392D/ | | |
| | | D399V/F405T/ | T366Y/Y407T | D399K/K439E | | |
| | | K409W | | | | |
| 0% Combination 385 | WT | Q347R/K360E/ | Q347E/S354Y/ | K392D/D399K/ | | |
| | | D399V/F405T/ | T366Y/Y407T | K409D | | |
| | | K409W | | | | |
| 0% Combination 386 | WT | Q347R/K360E/ | Q347E/S354Y/ | E356K/K392D/ | | |
| | | D399V/F405T/ | T366Y/Y407T | D399K/K409D | | |
| | | K409W | | | | |
| 0% Combination 387 | WT | Q347R/K360E/ | Q347E/S354Y/ | T394F/F405A | | |
| | | D399V/F405T/ | T366Y/Y407T | | | |
| | | K409W | | | | |
| 0% Combination 388 | WT | Q347R/K360E/ | Q347E/S354Y/ | K392L/T394W/ | | |
| | | D399V/F405T/ | T366Y/Y407T | F405A/Y407V | | |
| | | K409W | | | | |
| 0% Combination 389 | WT | Q347R/K360E/ | Q347E/S354Y/ | T350V/L351Y/ | | |
| | | D399V/F405T/ | T366Y/Y407T | T366L/K392L/ | | |
| | | K409W | | T394W/F405A/ | | |
| | | | | Y407V | | |
| 0% Combination 390 | WT | Q347R/K360E/ | E356K/K392D/ | T394F/F405A | | |
| | | D399V/F405T/ | D399K/K439E | | | |
| | | K409W | | | | |
| 0% Combination 391 | WT | Q347R/K360E/ | E356K/K392D/ | K392L/T394W/ | | |
| | | D399V/F405T/ | D399K/K439E | F405A/Y407V | | |
| | | K409W | | | | |
| 0% Combination 392 | WT | Q347R/K360E/ | E356K/K392D/ | T350V/L351Y/ | | |
| | | D399V/F405T/ | D399K/K439E | T366L/K392L/ | | |
| | | K409W | | T394W/F405A/ | | |
| | | | | Y407V | | |
| 0% Combination 393 | WT | Q347R/K360E/ | K392D/D399K/ | T394F/F405A | | |
| | | D399V/F405T/ | K409D | | | |
| | | K409W | | | | |
| 0% Combination 394 | WT | Q347R/K360E/ | K392D/D399K/ | K392L/T394W/ | | |
| | | D399V/F405T/ | K409D | F405A/Y407V | | |
| | | K409W | | | | |
| 0% Combination 395 | WT | Q347R/K360E/ | K392D/D399K/ | T350V/L351Y/ | | |
| | | D399V/F405T/ | K409D | T366L/K392L/ | | |
| | | K409W | | T394W/F405A/ | | |
| | | | | Y407V | | |
| 0% Combination 396 | WT | Q347R/K360E/ | E356K/K392D/ | T394F/F405A | | |
| | | D399V/F405T/ | D399K/K409D | | | |
| | | K409W | | | | |
| 0% Combination 397 | WT | Q347R/K360E/ | E356K/K392D/ | K392L/T394W/ | | |
| | | D399V/F405T/ | D399K/K409D | F405A/Y407V | | |
| | | K409W | | | | |
| 0% Combination 398 | WT | Q347R/K360E/ | E356K/K392D/ | T350V/L351Y/ | | |
| | | D399V/F405T/ | D399K/K409D | T366L/K392L/ | | |
| | | K409W | | T394W/F405A/ | | |
| | | | | Y407V | | |
| 0% Combination 399 | WT | E356K/D399K/ | Y349C/E357C/ | E345R/K360E/ | | |
| | | K409E/K439E | K392D/D399K | D399M/Y407A/ | | |
| | | | | K409V | | |
| 0% Combination 400 | WT | E356K/D399K/ | Y349C/E357C/ | T394F/F405A | | |
| | | K409E/K439E | K392D/D399K | | | |
| 0% Combination 401 | WT | E356K/D399K/ | Y349C/E357C/ | K392L/T394W/ | | |
| | | K409E/K439E | K392D/D399K | F405A/Y407V | | |
| 0% Combination 402 | WT | E356K/D399K/ | Y349C/E357C/ | T350V/L351Y/ | | |
| | | K409E/K439E | K392D/D399K | T366L/K392L/ | | |
| | | | | T394W/F405A/ | | |
| | | | | Y407V | | |
| 0% Combination 403 | WT | E356K/D399K/ | E345R/Q347R/ | T394F/F405A | | |
| | | K409E/K439E | K360D/T366V/ | | | |
| | | | D399M/Y407A/ | | | |
| | | | K409V | | | |
| 0% Combination 404 | WT | E356K/D399K/ | E345R/Q347R/ | K392L/T394W/ | | |
| | | K409E/K439E | K360D/T366V/ | F405A/Y407V | | |
| | | | D399M/Y407A/ | | | |
| | | | K409V | | | |
| 0% Combination 405 | WT | E356K/D399K/ | E345R/Q347R/ | T350V/L351Y/ | | |
| | | K409E/K439E | K360D/T366V/ | T366L/K392L/ | | |
| | | | D399M/Y407A/ | T394W/F405A/ | | |
| | | | K409V | Y407V | | |
| 0% Combination 406 | WT | E356K/D399K/ | Q347E/S354Y/ | E345R/K360E/ | | |
| | | K409E/K439E | T366Y/Y407T | D399M/Y407A/ | | |
| | | | | K409V | | |
| 0% Combination 407 | WT | E356K/D399K/ | Q347E/S354Y/ | T394F/F405A | | |
| | | K409E/K439E | T366Y/Y407T | | | |
| 0% Combination 408 | WT | E356K/D399K/ | Q347E/S354Y/ | K392L/T394W/ | | |
| | | K409E/K439E | T366Y/Y407T | F405A/Y407V | | |
| 0% Combination 409 | WT | E356K/D399K/ | Q347E/S354Y/ | T350V/L351Y/ | | |
| | | K409E/K439E | T366Y/Y407T | T366L/K392L/ | | |
| | | | | T394W/F405A/ | | |
| | | | | Y407V | | |
| 0% Combination 410 | WT | E356K/D399K/ | E345R/K360E/ | T394F/F405A | | |
| | | K409E/K439E | D399M/Y407A/ | | | |
| | | | K409V | | | |
| 0% Combination 411 | WT | E356K/D399K/ | E345R/K360E/ | K392L/T394W/ | | |
| | | K409E/K439E | D399M/Y407A/ | F405A/Y407V | | |
| | | | K409V | | | |
| 0% Combination 412 | WT | E356K/D399K/ | E345R/K360E/ | T350V/L351Y/ | | |
| | | K409E/K439E | D399M/Y407A/ | T366L/K392L/ | | |
| | | | K409V | T394W/F405A/ | | |
| | | | | Y407V | | |
| 0% Combination 413 | WT | Y349C/E357C/ | E356K/K392D/ | E345R/K360E/ | | |
| | | K392D/D399K | D399K/K439E | D399M/Y407A/ | | |
| | | | | K409V | | |
| 0% Combination 414 | WT | Y349C/E357C/ | E356K/K392D/ | T394F/F405A | | |
| | | K392D/D399K | D399K/K439E | | | |
| 0% Combination 415 | WT | Y349C/E357C/ | E356K/K392D/ | K392L/T394W/ | | |
| | | K392D/D399K | D399K/K439E | F405A/Y407V | | |
| 0% Combination 416 | WT | Y349C/E357C/ | E356K/K392D/ | T350V/L351Y/ | | |
| | | K392D/D399K | D399K/K439E | T366L/K392L/ | | |
| | | | | T394W/F405A/ | | |
| | | | | Y407V | | |
| 0% Combination 417 | WT | Y349C/E357C/ | E356K/K392D/ | E345R/K360E/ | | |
| | | K392D/D399K | D399K/K409D | D399M/Y407A/ | | |
| | | | | K409V | | |
| 0% Combination 418 | WT | Y349C/E357C/ | E356K/K392D/ | T394F/F405A | | |
| | | K392D/D399K | D399K/K409D | | | |
| 0% Combination 419 | WT | Y349C/E357C/ | E356K/K392D/ | K392L/T394W/ | | |
| | | K392D/D399K | D399K/K409D | F405A/Y407V | | |
| 0% Combination 420 | WT | Y349C/E357C/ | E356K/K392D/ | T350V/L351Y/ | | |
| | | K392D/D399K | D399K/K409D | T366L/K392L/ | | |
| | | | | T394W/F405A/ | | |
| | | | | Y407V | | |
| 0% Combination 421 | WT | Y349C/E357C/ | E345R/K360E/ | T394F/F405A | | |
| | | K392D/D399K | D399M/Y407A/ | | | |
| | | | K409V | | | |
| 0% Combination 422 | WT | Y349C/E357C/ | E345R/K360E/ | K392L/T394W/ | | |
| | | K392D/D399K | D399M/Y407A/ | F405A/Y407V | | |
| | | | K409V | | | |
| 0% Combination 423 | WT | Y349C/E357C/ | E345R/K360E/ | T350V/L351Y/ | | |
| | | K392D/D399K | D399M/Y407A/ | T366L/K392L/ | | |
| | | | K409V | T394W/F405A/ | | |
| | | | | Y407V | | |
| 0% Combination 424 | WT | E345R/Q347R/ | E356K/K392D/ | T394F/F405A | | |
| | | K360D/T366V/ | D399K/K439E | | | |
| | | D399M/Y407A/ | | | | |
| | | K409V | | | | |
| 0% Combination 425 | WT | E345R/Q347R/ | E356K/K392D/ | K392L/T394W/ | | |
| | | K360D/T366V/ | D399K/K439E | F405A/Y407V | | |
| | | D399M/Y407A/ | | | | |
| | | K409V | | | | |
| 0% Combination 426 | WT | E345R/Q347R/ | E356K/K392D/ | T350V/L351Y/ | | |
| | | K360D/T366V/ | D399K/K439E | T366L/K392L/ | | |
| | | D399M/Y407A/ | | T394W/F405A/ | | |
| | | K409V | | Y407V | | |
| 0% Combination 427 | WT | E345R/Q347R/ | K392D/D399K/ | T394F/F405A | | |
| | | K360D/T366V/ | K409D | | | |
| | | D399M/Y407A/ | | | | |
| | | K409V | | | | |
| 0% Combination 428 | WT | E345R/Q347R/ | K392D/D399K/ | K392L/T394W/ | | |
| | | K360D/T366V/ | K409D | F405A/Y407V | | |
| | | D399M/Y407A/ | | | | |
| | | K409V | | | | |
| 0% Combination 429 | WT | E345R/Q347R/ | K392D/D399K/ | T350V/L351Y/ | | |
| | | K360D/T366V/ | K409D | T366L/K392L/ | | |
| | | D399M/Y407A/ | | T394W/F405A/ | | |
| | | K409V | | Y407V | | |
| 0% Combination 430 | WT | E345R/Q347R/ | E356K/K392D/ | T394F/F405A | | |
| | | K360D/T366V/ | D399K/K409D | | | |
| | | D399M/Y407A/ | | | | |
| | | K409V | | | | |
| 0% Combination 431 | WT | E345R/Q347R/ | E356K/K392D/ | K392L/T394W/ | | |
| | | K360D/T366V/ | D399K/K409D | F405A/Y407V | | |
| | | D399M/Y407A/ | | | | |
| | | K409V | | | | |
| 0% Combination 432 | WT | E345R/Q347R/ | E356K/K392D/ | T350V/L351Y/ | | |
| | | K360D/T366V/ | D399K/K409D | T366L/K392L/ | | |
| | | D399M/Y407A/ | | T394W/F405A/ | | |
| | | K409V | | Y407V | | |
| 0% Combination 433 | WT | Q347E/S354Y/ | E356K/K392D/ | E345R/K360E/ | | |
| | | T366Y/Y407T | D399K/K439E | D399M/Y407A/ | | |
| | | | | K409V | | |
| 0% Combination 434 | WT | Q347E/S354Y/ | E356K/K392D/ | T394F/F405A | | |
| | | T366Y/Y407T | D399K/K439E | | | |
| 0% Combination 435 | WT | Q347E/S354Y/ | E356K/K392D/ | K392L/T394W/ | | |
| | | T366Y/Y407T | D399K/K439E | F405A/Y407V | | |
| 0% Combination 436 | WT | Q347E/S354Y/ | E356K/K392D/ | T350V/L351Y/ | | |
| | | T366Y/Y407T | D399K/K439E | T366L/K392L/ | | |
| | | | | T394W/F405A/ | | |
| | | | | Y407V | | |
| 0% Combination 437 | WT | Q347E/S354Y/ | K392D/D399K/ | T394F/F405A | | |
| | | T366Y/Y407T | K409D | | | |
| 0% Combination 438 | WT | Q347E/S354Y/ | K392D/D399K/ | K392L/T394W/ | | |
| | | T366Y/Y407T | K409D | F405A/Y407V | | |
| 0% Combination 439 | WT | Q347E/S354Y/ | K392D/D399K/ | T350V/L351Y/ | | |
| | | T366Y/Y407T | K409D | T366L/K392L/ | | |
| | | | | T394W/F405A/ | | |
| | | | | Y407V | | |
| 0% Combination 440 | WT | Q347E/S354Y/ | E356K/K392D/ | E345R/K360E/ | | |
| | | T366Y/Y407T | D399K/K409D | D399M/Y407A/ | | |
| | | | | K409V | | |
| 0% Combination 441 | WT | Q347E/S354Y/ | E356K/K392D/ | T394F/F405A | | |
| | | T366Y/Y407T | D399K/K409D | | | |
| 0% Combination 442 | WT | Q347E/S354Y/ | E356K/K392D/ | K392L/T394W/ | | |
| | | T366Y/Y407T | D399K/K409D | F405A/Y407V | | |
| 0% Combination 443 | WT | Q347E/S354Y/ | E356K/K392D/ | T350V/L351Y/ | | |
| | | T366Y/Y407T | D399K/K409D | T366L/K392L/ | | |
| | | | | T394W/F405A/ | | |
| | | | | Y407V | | |
| 0% Combination 444 | WT | Q347E/S354Y/ | E345R/K360E/ | T394F/F405A | | |
| | | T366Y/Y407T | D399M/Y407A/ | | | |
| | | | K409V | | | |
| 0% Combination 445 | WT | Q347E/S354Y/ | E345R/K360E/ | K392L/T394W/ | | |
| | | T366Y/Y407T | D399M/Y407A/ | F405A/Y407V | | |
| | | | K409V | | | |
| 0% Combination 446 | WT | Q347E/S354Y/ | E345R/K360E/ | T350V/L351Y/ | | |
| | | T366Y/Y407T | D399M/Y407A/ | T366L/K392L/ | | |
| | | | K409V | T394W/F405A/ | | |
| | | | | Y407V | | |
| 0% Combination 447 | WT | E356K/K392D/ | E345R/K360E/ | T394F/F405A | | |
| | | D399K/K439E | D399M/Y407A/ | | | |
| | | | K409V | | | |
| 0% Combination 448 | WT | E356K/K392D/ | E345R/K360E/ | K392L/T394W/ | | |
| | | D399K/K439E | D399M/Y407A/ | F405A/Y407V | | |
| | | | K409V | | | |
| 0% Combination 449 | WT | E356K/K392D/ | E345R/K360E/ | T350V/L351Y/ | | |
| | | D399K/K439E | D399M/Y407A/ | T366L/K392L/ | | |
| | | | K409V | T394W/F405A/ | | |
| | | | | Y407V | | |
| 0% Combination 450 | WT | E356K/K392D/ | E345R/K360E/ | T394F/F405A | | |
| | | D399K/K409D | D399M/Y407A/ | | | |
| | | | K409V | | | |
| 0% Combination 451 | WT | E356K/K392D/ | E345R/K360E/ | K392L/T394W/ | | |
| | | D399K/K409D | D399M/Y407A/ | F405A/Y407V | | |
| | | | K409V | | | |
| 0% Combination 452 | WT | E356K/K392D/ | E345R/K360E/ | T350V/L351Y/ | | |
| | | D399K/K409D | D399M/Y407A/ | T366L/K392L/ | | |
| | | | K409V | T394W/F405A/ | | |
| | | | | Y407V | | |
| 0% Combination 453 | T394W/F405A | T366W/L368A/ | Q347R/K360E/ | E356K/D399K/ | | |
| | | Y407V | D399V/F405T/ | K409E/K439E | | |
| | | | K409W | | | |
| 0% Combination 454 | T394W/F405A | T366W/L368A/ | Q347R/K360E/ | Y349C/E357C/ | | |
| | | Y407V | D399V/F405T/ | K392D/D399K | | |
| | | | K409W | | | |
| 0% Combination 455 | T394W/F405A | T366W/L368A/ | Q347R/K360E/ | E356K/K392D/ | | |
| | | Y407V | D399V/F405T/ | D399K/K439E | | |
| | | | K409W | | | |
| 0% Combination 456 | T394W/F405A | T366W/L368A/ | Q347R/K360E/ | K392D/D399K/ | | |
| | | Y407V | D399V/F405T/ | K409D | | |
| | | | K409W | | | |
| 0% Combination 457 | T394W/F405A | T366W/L368A/ | Q347R/K360E/ | E356K/K392D/ | | |
| | | Y407V | D399V/F405T/ | D399K/K409D | | |
| | | | K409W | | | |
| 0% Combination 458 | T394W/F405A | T366W/L368A/ | E356K/D399K/ | Y349C/E357C/ | | |
| | | Y407V | K409E/K439E | K392D/D399K | | |
| 0% Combination 459 | T394W/F405A | T366W/L368A/ | E356K/D399K/ | E345R/Q347R/ | | |
| | | Y407V | K409E/K439E | K360D/T366V/ | | |
| | | | | D399M/Y407A/ | | |
| | | | | K409V | | |
| 0% Combination 460 | T394W/F405A | T366W/L368A/ | E356K/D399K/ | E345R/K360E/ | | |
| | | Y407V | K409E/K439E | D399M/Y407A/ | | |
| | | | | K409V | | |
| 0% Combination 461 | T394W/F405A | T366W/L368A/ | Y349C/E357C/ | E356K/K392D/ | | |
| | | Y407V | K392D/D399K | D399K/K439E | | |
| 0% Combination 462 | T394W/F405A | T366W/L368A/ | Y349C/E357C/ | E356K/K392D/ | | |
| | | Y407V | K392D/D399K | D399K/K409D | | |
| 0% Combination 463 | T394W/F405A | T366W/L368A/ | Y349C/E357C/ | E345R/K360E/ | | |
| | | Y407V | K392D/D399K | D399M/Y407A/ | | |
| | | | | K409V | | |
| 0% Combination 464 | T394W/F405A | T366W/L368A/ | E345R/Q347R/ | E356K/K392D/ | | |
| | | Y407V | K360D/T366V/ | D399K/K439E | | |
| | | | D399M/Y407A/ | | | |
| | | | K409V | | | |
| 0% Combination 465 | T394W/F405A | T366W/L368A/ | E345R/Q347R/ | K392D/D399K/ | | |
| | | Y407V | K360D/T366V/ | K409D | | |
| | | | D399M/Y407A/ | | | |
| | | | K409V | | | |
| 0% Combination 466 | T394W/F405A | T366W/L368A/ | E345R/Q347R/ | E356K/K392D/ | | |
| | | Y407V | K360D/T366V/ | D399K/K409D | | |
| | | | D399M/Y407A/ | | | |
| | | | K409V | | | |
| 0% Combination 467 | T394W/F405A | T366W/L368A/ | E356K/K392D/ | E345R/K360E/ | | |
| | | Y407V | D399K/K439E | D399M/Y407A/ | | |
| | | | | K409V | | |
| 0% Combination 468 | T394W/F405A | T366W/L368A/ | E356K/K392D/ | E345R/K360E/ | | |
| | | Y407V | D399K/K409D | D399M/Y407A/ | | |
| | | | | K409V | | |
| 0% Combination 469 | T394W/F405A | Q347R/K360E/ | E356K/D399K/ | Y349C/E357C/ | | |
| | | D399V/F405T/ | K409E/K439E | K392D/D399K | | |
| | | K409W | | | | |
| 0% Combination 470 | T394W/F405A | Q347R/K360E/ | E356K/D399K/ | Q347E/S354Y/ | | |
| | | D399V/F405T/ | K409E/K439E | T366Y/Y407T | | |
| | | K409W | | | | |
| 0% Combination 471 | T394W/F405A | Q347R/K360E/ | Y349C/E357C/ | E356K/K392D/ | | |
| | | D399V/F405T/ | K392D/D399K | D399K/K439E | | |
| | | K409W | | | | |
| 0% Combination 472 | T394W/F405A | Q347R/K360E/ | Y349C/E357C/ | E356K/K392D/ | | |
| | | D399V/F405T/ | K392D/D399K | D399K/K409D | | |
| | | K409W | | | | |
| 0% Combination 473 | T394W/F405A | Q347R/K360E/ | Q347E/S354Y/ | E356K/K392D/ | | |
| | | D399V/F405T/ | T366Y/Y407T | D399K/K439E | | |
| | | K409W | | | | |
| 0% Combination 474 | T394W/F405A | Q347R/K360E/ | Q347E/S354Y/ | K392D/D399K/ | | |
| | | D399V/F405T/ | T366Y/Y407T | K409D | | |
| | | K409W | | | | |
| 0% Combination 475 | T394W/F405A | Q347R/K360E/ | Q347E/S354Y/ | E356K/K392D/ | | |
| | | D399V/F405T/ | T366Y/Y407T | D399K/K409D | | |
| | | K409W | | | | |
| 0% Combination 476 | T394W/F405A | E356K/D399K/ | Y349C/E357C/ | E345R/K360E/ | | |
| | | K409E/K439E | K392D/D399K | D399M/Y407A/ | | |
| | | | | K409V | | |
| 0% Combination 477 | T394W/F405A | E356K/D399K/ | Q347E/S354Y/ | E345R/K360E/ | | |
| | | K409E/K439E | T366Y/Y407T | D399M/Y407A/ | | |
| | | | | K409V | | |
| 0% Combination 478 | T394W/F405A | Y349C/E357C/ | E356K/K392D/ | E345R/K360E/ | | |
| | | K392D/D399K | D399K/K439E | D399M/Y407A/ | | |
| | | | | K409V | | |
| 0% Combination 479 | T394W/F405A | Y349C/E357C/ | E356K/K392D/ | E345R/K360E/ | | |
| | | K392D/D399K | D399K/K409D | D399M/Y407A/ | | |
| | | | | K409V | | |
| 0% Combination 480 | T394W/F405A | Q347E/S354Y/ | E356K/K392D/ | E345R/K360E/ | | |
| | | T366Y/Y407T | D399K/K439E | D399M/Y407A/ | | |
| | | | | K409V | | |
| 0% Combination 481 | T394W/F405A | Q347E/S354Y/ | E356K/K392D/ | E345R/K360E/ | | |
| | | T366Y/Y407T | D399K/K409D | D399M/Y407A/ | | |
| | | | | K409V | | |
| 0% Combination 482 | T366W/L368A/ | Q347R/K360E/ | E356K/D399K/ | Y349C/E357C/ | | |
| | Y407V | D399V/F405T/ | K409E/K439E | K392D/D399K | | |
| | | K409W | | | | |
| 0% Combination 483 | T366W/L368A/ | Q347R/K360E/ | E356K/D399K/ | T394F/F405A | | |
| | Y407V | D399V/F405T/ | K409E/K439E | | | |
| | | K409W | | | | |
| 0% Combination 484 | T366W/L368A/ | Q347R/K360E/ | E356K/D399K/ | K392L/T394W/ | | |
| | Y407V | D399V/F405T/ | K409E/K439E | F405A/Y407V | | |
| | | K409W | | | | |
| 0% Combination 485 | T366W/L368A/ | Q347R/K360E/ | E356K/D399K/ | T350V/L351Y/ | | |
| | Y407V | D399V/F405T/ | K409E/K439E | T366L/K392L/ | | |
| | | K409W | | T394W/F405A/ | | |
| | | | | Y407V | | |
| 0% Combination 486 | T366W/L368A/ | Q347R/K360E/ | Y349C/E357C/ | E356K/K392D/ | | |
| | Y407V | D399V/F405T/ | K392D/D399K | D399K/K439E | | |
| | | K409W | | | | |
| 0% Combination 487 | T366W/L368A/ | Q347R/K360E/ | Y349C/E357C/ | E356K/K392D/ | | |
| | Y407V | D399V/F405T/ | K392D/D399K | D399K/K409D | | |
| | | K409W | | | | |
| 0% Combination 488 | T366W/L368A/ | Q347R/K360E/ | Y349C/E357C/ | T394F/F405A | | |
| | Y407V | D399V/F405T/ | K392D/D399K | | | |
| | | K409W | | | | |
| 0% Combination 489 | T366W/L368A/ | Q347R/K360E/ | Y349C/E357C/ | K392L/T394W/ | | |
| | Y407V | D399V/F405T/ | K392D/D399K | F405A/Y407V | | |
| | | K409W | | | | |
| 0% Combination 490 | T366W/L368A/ | Q347R/K360E/ | Y349C/E357C/ | T350V/L351Y/ | | |
| | Y407V | D399V/F405T/ | K392D/D399K | T366L/K392L/ | | |
| | | K409W | | T394W/F405A/ | | |
| | | | | Y407V | | |
| 0% Combination 491 | T366W/L368A/ | Q347R/K360E/ | E356K/K392D/ | T394F/F405A | | |
| | Y407V | D399V/F405T/ | D399K/K439E | | | |
| | | K409W | | | | |
| 0% Combination 492 | T366W/L368A/ | Q347R/K360E/ | E356K/K392D/ | K392L/T394W/ | | |
| | Y407V | D399V/F405T/ | D399K/K439E | F405A/Y407V | | |
| | | K409W | | | | |
| 0% Combination 493 | T366W/L368A/ | Q347R/K360E/ | E356K/K392D/ | T350V/L351Y/ | | |
| | Y407V | D399V/F405T/ | D399K/K439E | T366L/K392L/ | | |
| | | K409W | | T394W/F405A/ | | |
| | | | | Y407V | | |
| 0% Combination 494 | T366W/L368A/ | Q347R/K360E/ | K392D/D399K/ | T394F/F405A | | |
| | Y407V | D399V/F405T/ | K409D | | | |
| | | K409W | | | | |
| 0% Combination 495 | T366W/L368A/ | Q347R/K360E/ | K392D/D399K/ | K392L/T394W/ | | |
| | Y407V | D399V/F405T/ | K409D | F405A/Y407V | | |
| | | K409W | | | | |
| 0% Combination 496 | T366W/L368A/ | Q347R/K360E/ | K392D/D399K/ | T350V/L351Y/ | | |
| | Y407V | D399V/F405T/ | K409D | T366L/K392L/ | | |
| | | K409W | | T394W/F405A/ | | |
| | | | | Y407V | | |
| 0% Combination 497 | T366W/L368A/ | Q347R/K360E/ | E356K/K392D/ | T394F/F405A | | |
| | Y407V | D399V/F405T/ | D399K/K409D | | | |
| | | K409W | | | | |
| 0% Combination 498 | T366W/L368A/ | Q347R/K360E/ | E356K/K392D/ | K392L/T394W/ | | |
| | Y407V | D399V/F405T/ | D399K/K409D | F405A/Y407V | | |
| | | K409W | | | | |
| 0% Combination 499 | T366W/L368A/ | Q347R/K360E/ | E356K/K392D/ | T350V/L351Y/ | | |
| | Y407V | D399V/F405T/ | D399K/K409D | T366L/K392L/ | | |
| | | K409W | | T394W/F405A/ | | |
| | | | | Y407V | | |
| 0% Combination 500 | T366W/L368A/ | E356K/D399K/ | Y349C/E357C/ | E345R/K360E/ | | |
| | Y407V | K409E/K439E | K392D/D399K | D399M/Y407A/ | | |
| | | | | K409V | | |
| 0% Combination 501 | T366W/L368A/ | E356K/D399K/ | Y349C/E357C/ | T394F/F405A | | |
| | Y407V | K409E/K439E | K392D/D399K | | | |
| 0% Combination 502 | T366W/L368A/ | E356K/D399K/ | Y349C/E357C/ | K392L/T394W/ | | |
| | Y407V | K409E/K439E | K392D/D399K | F405A/Y407V | | |
| 0% Combination 503 | T366W/L368A/ | E356K/D399K/ | Y349C/E357C/ | T350V/L351Y/ | | |
| | Y407V | K409E/K439E | K392D/D399K | T366L/K392L/ | | |
| | | | | T394W/F405A/ | | |
| | | | | Y407V | | |
| 0% Combination 504 | T366W/L368A/ | E356K/D399K/ | E345R/Q347R/ | T394F/F405A | | |
| | Y407V | K409E/K439E | K360D/T366V/ | | | |
| | | | D399M/Y407A/ | | | |
| | | | K409V | | | |
| 0% Combination 505 | T366W/L368A/ | E356K/D399K/ | E345R/Q347R/ | K392L/T394W/ | | |
| | Y407V | K409E/K439E | K360D/T366V/ | F405A/Y407V | | |
| | | | D399M/Y407A/ | | | |
| | | | K409V | | | |
| 0% Combination 506 | T366W/L368A/ | E356K/D399K/ | E345R/Q347R/ | T350V/L351Y/ | | |
| | Y407V | K409E/K439E | K360D/T366V/ | T366L/K392L/ | | |
| | | | D399M/Y407A/ | T394W/F405A/ | | |
| | | | K409V | Y407V | | |
| 0% Combination 507 | T366W/L368A/ | E356K/D399K/ | E345R/K360E/ | T394F/F405A | | |
| | Y407V | K409E/K439E | D399M/Y407A/ | | | |
| | | | K409V | | | |
| 0% Combination 508 | T366W/L368A/ | E356K/D399K/ | E345R/K360E/ | K392L/T394W/ | | |
| | Y407V | K409E/K439E | D399M/Y407A/ | F405A/Y407V | | |
| | | | K409V | | | |
| 0% Combination 509 | T366W/L368A/ | E356K/D399K/ | E345R/K360E/ | T350V/L351Y/ | | |
| | Y407V | K409E/K439E | D399M/Y407A/ | T366L/K392L/ | | |
| | | | K409V | T394W/F405A/ | | |
| | | | | Y407V | | |
| 0% Combination 510 | T366W/L368A/ | Y349C/E357C/ | E356K/K392D/ | E345R/K360E/ | | |
| | Y407V | K392D/D399K | D399K/K439E | D399M/Y407A/ | | |
| | | | | K409V | | |
| 0% Combination 511 | T366W/L368A/ | Y349C/E357C/ | E356K/K392D/ | T394F/F405A | | |
| | Y407V | K392D/D399K | D399K/K439E | | | |
| 0% Combination 512 | T366W/L368A/ | Y349C/E357C/ | E356K/K392D/ | K392L/T394W/ | | |
| | Y407V | K392D/D399K | D399K/K439E | F405A/Y407V | | |
| 0% Combination 513 | T366W/L368A/ | Y349C/E357C/ | E356K/K392D/ | T350V/L351Y/ | | |
| | Y407V | K392D/D399K | D399K/K439E | T366L/K392L/ | | |
| | | | | T394W/F405A/ | | |
| | | | | Y407V | | |
| 0% Combination 514 | T366W/L368A/ | Y349C/E357C/ | E356K/K392D/ | E345R/K360E/ | | |
| | Y407V | K392D/D399K | D399K/K409D | D399M/Y407A/ | | |
| | | | | K409V | | |
| 0% Combination 515 | T366W/L368A/ | Y349C/E357C/ | E356K/K392D/ | T394F/F405A | | |
| | Y407V | K392D/D399K | D399K/K409D | | | |
| 0% Combination 516 | T366W/L368A/ | Y349C/E357C/ | E356K/K392D/ | K392L/T394W/ | | |
| | Y407V | K392D/D399K | D399K/K409D | F405A/Y407V | | |
| 0% Combination 517 | T366W/L368A/ | Y349C/E357C/ | E356K/K392D/ | T350V/L351Y/ | | |
| | Y407V | K392D/D399K | D399K/K409D | T366L/K392L/ | | |
| | | | | T394W/F405A/ | | |
| | | | | Y407V | | |
| 0% Combination 518 | T366W/L368A/ | Y349C/E357C/ | E345R/K360E/ | T394F/F405A | | |
| | Y407V | K392D/D399K | D399M/Y407A/ | | | |
| | | | K409V | | | |
| 0% Combination 519 | T366W/L368A/ | Y349C/E357C/ | E345R/K360E/ | K392L/T394W/ | | |
| | Y407V | K392D/D399K | D399M/Y407A/ | F405A/Y407V | | |
| | | | K409V | | | |
| 0% Combination 520 | T366W/L368A/ | Y349C/E357C/ | E345R/K360E/ | T350V/L351Y/ | | |
| | Y407V | K392D/D399K | D399M/Y407A/ | T366L/K392L/ | | |
| | | | K409V | T394W/F405A/ | | |
| | | | | Y407V | | |
| 0% Combination 521 | T366W/L368A/ | E345R/Q347R/ | E356K/K392D/ | T394F/F405A | | |
| | Y407V | K360D/T366V/ | D399K/K439E | | | |
| | | D399M/Y407A/ | | | | |
| | | K409V | | | | |
| 0% Combination 522 | T366W/L368A/ | E345R/Q347R/ | E356K/K392D/ | K392L/T394W/ | | |
| | Y407V | K360D/T366V/ | D399K/K439E | F405A/Y407V | | |
| | | D399M/Y407A/ | | | | |
| | | K409V | | | | |
| 0% Combination 523 | T366W/L368A/ | E345R/Q347R/ | E356K/K392D/ | T350V/L351Y/ | | |
| | Y407V | K360D/T366V/ | D399K/K439E | T366L/K392L/ | | |
| | | D399M/Y407A/ | | T394W/F405A/ | | |
| | | K409V | | Y407V | | |
| 0% Combination 524 | T366W/L368A/ | E345R/Q347R/ | K392D/D399K/ | T394F/F405A | | |
| | Y407V | K360D/T366V/ | K409D | | | |
| | | D399M/Y407A/ | | | | |
| | | K409V | | | | |
| 0% Combination 525 | T366W/L368A/ | E345R/Q347R/ | K392D/D399K/ | K392L/T394W/ | | |
| | Y407V | K360D/T366V/ | K409D | F405A/Y407V | | |
| | | D399M/Y407A/ | | | | |
| | | K409V | | | | |
| 0% Combination 526 | T366W/L368A/ | E345R/Q347R/ | K392D/D399K/ | T350V/L351Y/ | | |
| | Y407V | K360D/T366V/ | K409D | T366L/K392L/ | | |
| | | D399M/Y407A/ | | T394W/F405A/ | | |
| | | K409V | | Y407V | | |
| 0% Combination 527 | T366W/L368A/ | E345R/Q347R/ | E356K/K392D/ | T394F/F405A | | |
| | Y407V | K360D/T366V/ | D399K/K409D | | | |
| | | D399M/Y407A/ | | | | |
| | | K409V | | | | |
| 0% Combination 528 | T366W/L368A/ | E345R/Q347R/ | E356K/K392D/ | K392L/T394W/ | | |
| | Y407V | K360D/T366V/ | D399K/K409D | F405A/Y407V | | |
| | | D399M/Y407A/ | | | | |
| | | K409V | | | | |
| 0% Combination 529 | T366W/L368A/ | E345R/Q347R/ | E356K/K392D/ | T350V/L351Y/ | | |
| | Y407V | K360D/T366V/ | D399K/K409D | T366L/K392L/ | | |
| | | D399M/Y407A/ | | T394W/F405A/ | | |
| | | K409V | | Y407V | | |
| 0% Combination 530 | T366W/L368A/ | E356K/K392D/ | E345R/K360E/ | T394F/F405A | | |
| | Y407V | D399K/K439E | D399M/Y407A/ | | | |
| | | | K409V | | | |
| 0% Combination 531 | T366W/L368A/ | E356K/K392D/ | E345R/K360E/ | K392L/T394W/ | | |
| | Y407V | D399K/K439E | D399M/Y407A/ | F405A/Y407V | | |
| | | | K409V | | | |
| 0% Combination 532 | T366W/L368A/ | E356K/K392D/ | E345R/K360E/ | T350V/L351Y/ | | |
| | Y407V | D399K/K439E | D399M/Y407A/ | T366L/K392L/ | | |
| | | | K409V | T394W/F405A/ | | |
| | | | | Y407V | | |
| 0% Combination 533 | T366W/L368A/ | E356K/K392D/ | E345R/K360E/ | T394F/F405A | | |
| | Y407V | D399K/K409D | D399M/Y407A/ | | | |
| | | | K409V | | | |
| 0% Combination 534 | T366W/L368A/ | E356K/K392D/ | E345R/K360E/ | K392L/T394W/ | | |
| | Y407V | D399K/K409D | D399M/Y407A/ | F405A/Y407V | | |
| | | | K409V | | | |
| 0% Combination 535 | T366W/L368A/ | E356K/K392D/ | E345R/K360E/ | T350V/L351Y/ | | |
| | Y407V | D399K/K409D | D399M/Y407A/ | T366L/K392L/ | | |
| | | | K409V | T394W/F405A/ | | |
| | | | | Y407V | | |
| 0% Combination 536 | Q347R/K360E/ | E356K/D399K/ | Y349C/E357C/ | T394F/F405A | | |
| | D399V/F405T/ | K409E/K439E | K392D/D399K | | | |
| | K409W | | | | | |
| 0% Combination 537 | Q347R/K360E/ | E356K/D399K/ | Y349C/E357C/ | K392L/T394W/ | | |
| | D399V/F405T/ | K409E/K439E | K392D/D399K | F405A/Y407V | | |
| | K409W | | | | | |
| 0% Combination 538 | Q347R/K360E/ | E356K/D399K/ | Y349C/E357C/ | T350V/L351Y/ | | |
| | D399V/F405T/ | K409E/K439E | K392D/D399K | T366L/K392L/ | | |
| | K409W | | | T394W/F405A/ | | |
| | | | | Y407V | | |
| 0% Combination 539 | Q347R/K360E/ | E356K/D399K/ | Q347E/S354Y/ | T394F/F405A | | |
| | D399V/F405T/ | K409E/K439E | T366Y/Y407T | | | |
| | K409W | | | | | |
| 0% Combination 540 | Q347R/K360E/ | E356K/D399K/ | Q347E/S354Y/ | K392L/T394W/ | | |
| | D399V/F405T/ | K409E/K439E | T366Y/Y407T | F405A/Y407V | | |
| | K409W | | | | | |
| 0% Combination 541 | Q347R/K360E/ | E356K/D399K/ | Q347E/S354Y/ | T350V/L351Y/ | | |
| | D399V/F405T/ | K409E/K439E | T366Y/Y407T | T366L/K392L/ | | |
| | K409W | | | T394W/F405A/ | | |
| | | | | Y407V | | |
| 0% Combination 542 | Q347R/K360E/ | Y349C/E357C/ | E356K/K392D/ | T394F/F405A | | |
| | D399V/F405T/ | K392D/D399K | D399K/K439E | | | |
| | K409W | | | | | |
| 0% Combination 543 | Q347R/K360E/ | Y349C/E357C/ | E356K/K392D/ | K392L/T394W/ | | |
| | D399V/F405T/ | K392D/D399K | D399K/K439E | F405A/Y407V | | |
| | K409W | | | | | |
| 0% Combination 544 | Q347R/K360E/ | Y349C/E357C/ | E356K/K392D/ | T350V/L351Y/ | | |
| | D399V/F405T/ | K392D/D399K | D399K/K439E | T366L/K392L/ | | |
| | K409W | | | T394W/F405A/ | | |
| | | | | Y407V | | |
| 0% Combination 545 | Q347R/K360E/ | Y349C/E357C/ | E356K/K392D/ | T394F/F405A | | |
| | D399V/F405T/ | K392D/D399K | D399K/K409D | | | |
| | K409W | | | | | |
| 0% Combination 546 | Q347R/K360E/ | Y349C/E357C/ | E356K/K392D/ | K392L/T394W/ | | |
| | D399V/F405T/ | K392D/D399K | D399K/K409D | F405A/Y407V | | |
| | K409W | | | | | |
| 0% Combination 547 | Q347R/K360E/ | Y349C/E357C/ | E356K/K392D/ | T350V/L351Y/ | | |
| | D399V/F405T/ | K392D/D399K | D399K/K409D | T366L/K392L/ | | |
| | K409W | | | T394W/F405A/ | | |
| | | | | Y407V | | |
| 0% Combination 548 | Q347R/K360E/ | Q347E/S354Y/ | E356K/K392D/ | T394F/F405A | | |
| | D399V/F405T/ | T366Y/Y407T | D399K/K439E | | | |
| | K409W | | | | | |
| 0% Combination 549 | Q347R/K360E/ | Q347E/S354Y/ | E356K/K392D/ | K392L/T394W/ | | |
| | D399V/F405T/ | T366Y/Y407T | D399K/K439E | F405A/Y407V | | |
| | K409W | | | | | |
| 0% Combination 550 | Q347R/K360E/ | Q347E/S354Y/ | E356K/K392D/ | T350V/L351Y/ | | |
| | D399V/F405T/ | T366Y/Y407T | D399K/K439E | T366L/K392L/ | | |
| | K409W | | | T394W/F405A/ | | |
| | | | | Y407V | | |
| 0% Combination 551 | Q347R/K360E/ | Q347E/S354Y/ | K392D/D399K/ | T394F/F405A | | |
| | D399V/F405T/ | T366Y/Y407T | K409D | | | |
| | K409W | | | | | |
| 0% Combination 552 | Q347R/K360E/ | Q347E/S354Y/ | K392D/D399K/ | K392L/T394W/ | | |
| | D399V/F405T/ | T366Y/Y407T | K409D | F405A/Y407V | | |
| | K409W | | | | | |
| 0% Combination 553 | Q347R/K360E/ | Q347E/S354Y/ | K392D/D399K/ | T350V/L351Y/ | | |
| | D399V/F405T/ | T366Y/Y407T | K409D | T366L/K392L/ | | |
| | K409W | | | T394W/F405A/ | | |
| | | | | Y407V | | |
| 0% Combination 554 | Q347R/K360E/ | Q347E/S354Y/ | E356K/K392D/ | T394F/F405A | | |
| | D399V/F405T/ | T366Y/Y407T | D399K/K409D | | | |
| | K409W | | | | | |
| 0% Combination 555 | Q347R/K360E/ | Q347E/S354Y/ | E356K/K392D/ | K392L/T394W/ | | |
| | D399V/F405T/ | T366Y/Y407T | D399K/K409D | F405A/Y407V | | |
| | K409W | | | | | |
| 0% Combination 556 | Q347R/K360E/ | Q347E/S354Y/ | E356K/K392D/ | T350V/L351Y/ | | |
| | D399V/F405T/ | T366Y/Y407T | D399K/K409D | T366L/K392L/ | | |
| | K409W | | | T394W/F405A/ | | |
| | | | | Y407V | | |
| 0% Combination 557 | E356K/D399K/ | Y349C/E357C/ | E345R/K360E/ | T394F/F405A | | |
| | K409E/K439E | K392D/D399K | D399M/Y407A/ | | | |
| | | | K409V | | | |
| 0% Combination 558 | E356K/D399K/ | Y349C/E357C/ | E345R/K360E/ | K392L/T394W/ | | |
| | K409E/K439E | K392D/D399K | D399M/Y407A/ | F405A/Y407V | | |
| | | | K409V | | | |
| 0% Combination 559 | E356K/D399K/ | Y349C/E357C/ | E345R/K360E/ | T350V/L351Y/ | | |
| | K409E/K439E | K392D/D399K | D399M/Y407A/ | T366L/K392L/ | | |
| | | | K409V | T394W/F405A/ | | |
| | | | | Y407V | | |
| 0% Combination 560 | E356K/D399K/ | Q347E/S354Y/ | E345R/K360E/ | T394F/F405A | | |
| | K409E/K439E | T366Y/Y407T | D399M/Y407A/ | | | |
| | | | K409V | | | |
| 0% Combination 561 | E356K/D399K/ | Q347E/S354Y/ | E345R/K360E/ | K392L/T394W/ | | |
| | K409E/K439E | T366Y/Y407T | D399M/Y407A/ | F405A/Y407V | | |
| | | | K409V | | | |
| 0% Combination 562 | E356K/D399K/ | Q347E/S354Y/ | E345R/K360E/ | T350V/L351Y/ | | |
| | K409E/K439E | T366Y/Y407T | D399M/Y407A/ | T366L/K392L/ | | |
| | | | K409V | T394W/F405A/ | | |
| | | | | Y407V | | |
| 0% Combination 563 | Y349C/E357C/ | E356K/K392D/ | E345R/K360E/ | T394F/F405A | | |
| | K392D/D399K | D399K/K439E | D399M/Y407A/ | | | |
| | | | K409V | | | |
| 0% Combination 564 | Y349C/E357C/ | E356K/K392D/ | E345R/K360E/ | K392L/T394W/ | | |
| | K392D/D399K | D399K/K439E | D399M/Y407A/ | F405A/Y407V | | |
| | | | K409V | | | |
| 0% Combination 565 | Y349C/E357C/ | E356K/K392D/ | E345R/K360E/ | T350V/L351Y/ | | |
| | K392D/D399K | D399K/K439E | D399M/Y407A/ | T366L/K392L/ | | |
| | | | K409V | T394W/F405A/ | | |
| | | | | Y407V | | |
| 0% Combination 566 | Y349C/E357C/ | E356K/K392D/ | E345R/K360E/ | T394F/F405A | | |
| | K392D/D399K | D399K/K409D | D399M/Y407A/ | | | |
| | | | K409V | | | |
| 0% Combination 567 | Y349C/E357C/ | E356K/K392D/ | E345R/K360E/ | K392L/T394W/ | | |
| | K392D/D399K | D399K/K409D | D399M/Y407A/ | F405A/Y407V | | |
| | | | K409V | | | |
| 0% Combination 568 | Y349C/E357C/ | E356K/K392D/ | E345R/K360E/ | T350V/L351Y/ | | |
| | K392D/D399K | D399K/K409D | D399M/Y407A/ | T366L/K392L/ | | |
| | | | K409V | T394W/F405A/ | | |
| | | | | Y407V | | |
| 0% Combination 569 | Q347E/S354Y/ | E356K/K392D/ | E345R/K360E/ | T394F/F405A | | |
| | T366Y/Y407T | D399K/K439E | D399M/Y407A/ | | | |
| | | | K409V | | | |
| 0% Combination 570 | Q347E/S354Y/ | E356K/K392D/ | E345R/K360E/ | K392L/T394W/ | | |
| | T366Y/Y407T | D399K/K439E | D399M/Y407A/ | F405A/Y407V | | |
| | | | K409V | | | |
| 0% Combination 571 | Q347E/S354Y/ | E356K/K392D/ | E345R/K360E/ | T350V/L351Y/ | | |
| | T366Y/Y407T | D399K/K439E | D399M/Y407A/ | T366L/K392L/ | | |
| | | | K409V | T394W/F405A/ | | |
| | | | | Y407V | | |
| 0% Combination 572 | Q347E/S354Y/ | E356K/K392D/ | E345R/K360E/ | T394F/F405A | | |
| | T366Y/Y407T | D399K/K409D | D399M/Y407A/ | | | |
| | | | K409V | | | |
| 0% Combination 573 | Q347E/S354Y/ | E356K/K392D/ | E345R/K360E/ | K392L/T394W/ | | |
| | T366Y/Y407T | D399K/K409D | D399M/Y407A/ | F405A/Y407V | | |
| | | | K409V | | | |
| 0% Combination 574 | Q347E/S354Y/ | E356K/K392D/ | E345R/K360E/ | T350V/L351Y/ | | |
| | T366Y/Y407T | D399K/K409D | D399M/Y407A/ | T366L/K392L/ | | |
| | | | K409V | T394W/F405A/ | | |
| | | | | Y407V | | |
| 0% Combination 575 | WT | T394W/F405A | T366W/L368A/ | Q347R/K360E/ | E356K/D399K/ | |
| | | | Y407V | D399V/F405T/ | K409E/K439E | |
| | | | | K409W | | |
| 0% Combination 576 | WT | T394W/F405A | T366W/L368A/ | Q347R/K360E/ | Y349C/E357C/ | |
| | | | Y407V | D399V/F405T/ | K392D/D399K | |
| | | | | K409W | | |
| 0% Combination 577 | WT | T394W/F405A | T366W/L368A/ | Q347R/K360E/ | E356K/K392D/ | |
| | | | Y407V | D399V/F405T/ | D399K/K439E | |
| | | | | K409W | | |
| 0% Combination 578 | WT | T394W/F405A | T366W/L368A/ | Q347R/K360E/ | K392D/D399K/ | |
| | | | Y407V | D399V/F405T/ | K409D | |
| | | | | K409W | | |
| 0% Combination 579 | WT | T394W/F405A | T366W/L368A/ | Q347R/K360E/ | E356K/K392D/ | |
| | | | Y407V | D399V/F405T/ | D399K/K409D | |
| | | | | K409W | | |
| 0% Combination 580 | WT | T394W/F405A | T366W/L368A/ | E356K/D399K/ | Y349C/E357C/ | |
| | | | Y407V | K409E/K439E | K392D/D399K | |
| 0% Combination 581 | WT | T394W/F405A | T366W/L368A/ | E356K/D399K/ | E345R/Q347R/ | |
| | | | Y407V | K409E/K439E | K360D/T366V/ | |
| | | | | | D399M/Y407A/ | |
| | | | | | K409V | |
| 0% Combination 582 | WT | T394W/F405A | T366W/L368A/ | E356K/D399K/ | E345R/K360E/ | |
| | | | Y407V | K409E/K439E | D399M/Y407A/ | |
| | | | | | K409V | |
| 0% Combination 583 | WT | T394W/F405A | T366W/L368A/ | Y349C/E357C/ | E356K/K392D/ | |
| | | | Y407V | K392D/D399K | D399K/K439E | |
| 0% Combination 584 | WT | T394W/F405A | T366W/L368A/ | Y349C/E357C/ | E356K/K392D/ | |
| | | | Y407V | K392D/D399K | D399K/K409D | |
| 0% Combination 585 | WT | T394W/F405A | T366W/L368A/ | Y349C/E357C/ | E345R/K360E/ | |
| | | | Y407V | K392D/D399K | D399M/Y407A/ | |
| | | | | | K409V | |
| 0% Combination 586 | WT | T394W/F405A | T366W/L368A/ | E345R/Q347R/ | E356K/K392D/ | |
| | | | Y407V | K360D/T366V/ | D399K/K439E | |
| | | | | D399M/Y407A/ | | |
| | | | | K409V | | |
| 0% Combination 587 | WT | T394W/F405A | T366W/L368A/ | E345R/Q347R/ | K392D/D399K/ | |
| | | | Y407V | K360D/T366V/ | K409D | |
| | | | | D399M/Y407A/ | | |
| | | | | K409V | | |
| 0% Combination 588 | WT | T394W/F405A | T366W/L368A/ | E345R/Q347R/ | E356K/K392D/ | |
| | | | Y407V | K360D/T366V/ | D399K/K409D | |
| | | | | D399M/Y407A/ | | |
| | | | | K409V | | |
| 0% Combination 589 | WT | T394W/F405A | T366W/L368A/ | E356K/K392D/ | E345R/K360E/ | |
| | | | Y407V | D399K/K439E | D399M/Y407A/ | |
| | | | | | K409V | |
| 0% Combination 590 | WT | T394W/F405A | T366W/L368A/ | E356K/K392D/ | E345R/K360E/ | |
| | | | Y407V | D399K/K409D | D399M/Y407A/ | |
| | | | | | K409V | |
| 0% Combination 591 | WT | T394W/F405A | Q347R/K360E/ | E356K/D399K/ | Y349C/E357C/ | |
| | | | D399V/F405T/ | K409E/K439E | K392D/D399K | |
| | | | K409W | | | |
| 0% Combination 592 | WT | T394W/F405A | Q347R/K360E/ | E356K/D399K/ | Q347E/S354Y/ | |
| | | | D399V/F405T/ | K409E/K439E | T366Y/Y407T | |
| | | | K409W | | | |
| 0% Combination 593 | WT | T394W/F405A | Q347R/K360E/ | Y349C/E357C/ | E356K/K392D/ | |
| | | | D399V/F405T/ | K392D/D399K | D399K/K439E | |
| | | | K409W | | | |
| 0% Combination 594 | WT | T394W/F405A | Q347R/K360E/ | Y349C/E357C/ | E356K/K392D/ | |
| | | | D399V/F405T/ | K392D/D399K | D399K/K409D | |
| | | | K409W | | | |
| 0% Combination 595 | WT | T394W/F405A | Q347R/K360E/ | Q347E/S354Y/ | E356K/K392D/ | |
| | | | D399V/F405T/ | T366Y/Y407T | D399K/K439E | |
| | | | K409W | | | |
| 0% Combination 596 | WT | T394W/F405A | Q347R/K360E/ | Q347E/S354Y/ | K392D/D399K/ | |
| | | | D399V/F405T/ | T366Y/Y407T | K409D | |
| | | | K409W | | | |
| 0% Combination 597 | WT | T394W/F405A | Q347R/K360E/ | Q347E/S354Y/ | E356K/K392D/ | |
| | | | D399V/F405T/ | T366Y/Y407T | D399K/K409D | |
| | | | K409W | | | |
| 0% Combination 598 | WT | T394W/F405A | E356K/D399K/ | Y349C/E357C/ | E345R/K360E/ | |
| | | | K409E/K439E | K392D/D399K | D399M/Y407A/ | |
| | | | | | K409V | |
| 0% Combination 599 | WT | T394W/F405A | E356K/D399K/ | Q347E/S354Y/ | E345R/K360E/ | |
| | | | K409E/K439E | T366Y/Y407T | D399M/Y407A/ | |
| | | | | | K409V | |
| 0% Combination 600 | WT | T394W/F405A | Y349C/E357C/ | E356K/K392D/ | E345R/K360E/ | |
| | | | K392D/D399K | D399K/K439E | D399M/Y407A/ | |
| | | | | | K409V | |
| 0% Combination 601 | WT | T394W/F405A | Y349C/E357C/ | E356K/K392D/ | E345R/K360E/ | |
| | | | K392D/D399K | D399K/K409D | D399M/Y407A/ | |
| | | | | | K409V | |
| 0% Combination 602 | WT | T394W/F405A | Q347E/S354Y/ | E356K/K392D/ | E345R/K360E/ | |
| | | | T366Y/Y407T | D399K/K439E | D399M/Y407A/ | |
| | | | | | K409V | |
| 0% Combination 603 | WT | T394W/F405A | Q347E/S354Y/ | E356K/K392D/ | E345R/K360E/ | |
| | | | T366Y/Y407T | D399K/K409D | D399M/Y407A/ | |
| | | | | | K409V | |
| 0% Combination 604 | WT | T366W/L368A/ | Q347R/K360E/ | E356K/D399K/ | Y349C/E357C/ | |
| | | Y407V | D399V/F405T/ | K409E/K439E | K392D/D399K | |
| | | | K409W | | | |
| 0% Combination 605 | WT | T366W/L368A/ | Q347R/K360E/ | E356K/D399K/ | T394F/F405A | |
| | | Y407V | D399V/F405T/ | K409E/K439E | | |
| | | | K409W | | | |
| 0% Combination 606 | WT | T366W/L368A/ | Q347R/K360E/ | E356K/D399K/ | K392L/T394W/ | |
| | | Y407V | D399V/F405T/ | K409E/K439E | F405A/Y407V | |
| | | | K409W | | | |
| 0% Combination 607 | WT | T366W/L368A/ | Q347R/K360E/ | E356K/D399K/ | T350V/L351Y/ | |
| | | Y407V | D399V/F405T/ | K409E/K439E | T366L/K392L/ | |
| | | | K409W | | T394W/F405A/ | |
| | | | | | Y407V | |
| 0% Combination 608 | WT | T366W/L368A/ | Q347R/K360E/ | Y349C/E357C/ | E356K/K392D/ | |
| | | Y407V | D399V/F405T/ | K392D/D399K | D399K/K439E | |
| | | | K409W | | | |
| 0% Combination 609 | WT | T366W/L368A/ | Q347R/K360E/ | Y349C/E357C/ | E356K/K392D/ | |
| | | Y407V | D399V/F405T/ | K392D/D399K | D399K/K409D | |
| | | | K409W | | | |
| 0% Combination 610 | WT | T366W/L368A/ | Q347R/K360E/ | Y349C/E357C/ | T394F/F405A | |
| | | Y407V | D399V/F405T/ | K392D/D399K | | |
| | | | K409W | | | |
| 0% Combination 611 | WT | T366W/L368A/ | Q347R/K360E/ | Y349C/E357C/ | K392L/T394W/ | |
| | | Y407V | D399V/F405T/ | K392D/D399K | F405A/Y407V | |
| | | | K409W | | | |
| 0% Combination 612 | WT | T366W/L368A/ | Q347R/K360E/ | Y349C/E357C/ | T350V/L351Y/ | |
| | | Y407V | D399V/F405T/ | K392D/D399K | T366L/K392L/ | |
| | | | K409W | | T394W/F405A/ | |
| | | | | | Y407V | |
| 0% Combination 613 | WT | T366W/L368A/ | Q347R/K360E/ | E356K/K392D/ | T394F/F405A | |
| | | Y407V | D399V/F405T/ | D399K/K439E | | |
| | | | K409W | | | |
| 0% Combination 614 | WT | T366W/L368A/ | Q347R/K360E/ | E356K/K392D/ | K392L/T394W/ | |
| | | Y407V | D399V/F405T/ | D399K/K439E | F405A/Y407V | |
| | | | K409W | | | |
| 0% Combination 615 | WT | T366W/L368A/ | Q347R/K360E/ | E356K/K392D/ | T350V/L351Y/ | |
| | | Y407V | D399V/F405T/ | D399K/K439E | T366L/K392L/ | |
| | | | K409W | | T394W/F405A/ | |
| | | | | | Y407V | |
| 0% Combination 616 | WT | T366W/L368A/ | Q347R/K360E/ | K392D/D399K/ | T394F/F405A | |
| | | Y407V | D399V/F405T/ | K409D | | |
| | | | K409W | | | |
| 0% Combination 617 | WT | T366W/L368A/ | Q347R/K360E/ | K392D/D399K/ | K392L/T394W/ | |
| | | Y407V | D399V/F405T/ | K409D | F405A/Y407V | |
| | | | K409W | | | |
| 0% Combination 618 | WT | T366W/L368A/ | Q347R/K360E/ | K392D/D399K/ | T350V/L351Y/ | |
| | | Y407V | D399V/F405T/ | K409D | T366L/K392L/ | |
| | | | K409W | | T394W/F405A/ | |
| | | | | | Y407V | |
| 0% Combination 619 | WT | T366W/L368A/ | Q347R/K360E/ | E356K/K392D/ | T394F/F405A | |
| | | Y407V | D399V/F405T/ | D399K/K409D | | |
| | | | K409W | | | |
| 0% Combination 620 | WT | T366W/L368A/ | Q347R/K360E/ | E356K/K392D/ | K392L/T394W/ | |
| | | Y407V | D399V/F405T/ | D399K/K409D | F405A/Y407V | |
| | | | K409W | | | |
| 0% Combination 621 | WT | T366W/L368A/ | Q347R/K360E/ | E356K/K392D/ | T350V/L351Y/ | |
| | | Y407V | D399V/F405T/ | D399K/K409D | T366L/K392L/ | |
| | | | K409W | | T394W/F405A/ | |
| | | | | | Y407V | |
| 0% Combination 622 | WT | T366W/L368A/ | E356K/D399K/ | Y349C/E357C/ | E345R/K360E/ | |
| | | Y407V | K409E/K439E | K392D/D399K | D399M/Y407A/ | |
| | | | | | K409V | |
| 0% Combination 623 | WT | T366W/L368A/ | E356K/D399K/ | Y349C/E357C/ | T394F/F405A | |
| | | Y407V | K409E/K439E | K392D/D399K | | |
| 0% Combination 624 | WT | T366W/L368A/ | E356K/D399K/ | Y349C/E357C/ | K392L/T394W/ | |
| | | Y407V | K409E/K439E | K392D/D399K | F405A/Y407V | |
| 0% Combination 625 | WT | T366W/L368A/ | E356K/D399K/ | Y349C/E357C/ | T350V/L351Y/ | |
| | | Y407V | K409E/K439E | K392D/D399K | T366L/K392L/ | |
| | | | | | T394W/F405A/ | |
| | | | | | Y407V | |
| 0% Combination 626 | WT | T366W/L368A/ | E356K/D399K/ | E345R/Q347R/ | T394F/F405A | |
| | | Y407V | K409E/K439E | K360D/T366V/ | | |
| | | | | D399M/Y407A/ | | |
| | | | | K409V | | |
| 0% Combination 627 | WT | T366W/L368A/ | E356K/D399K/ | E345R/Q347R/ | K392L/T394W/ | |
| | | Y407V | K409E/K439E | K360D/T366V/ | F405A/Y407V | |
| | | | | D399M/Y407A/ | | |
| | | | | K409V | | |
| 0% Combination 628 | WT | T366W/L368A/ | E356K/D399K/ | E345R/Q347R/ | T350V/L351Y/ | |
| | | Y407V | K409E/K439E | K360D/T366V/ | T366L/K392L/ | |
| | | | | D399M/Y407A/ | T394W/F405A/ | |
| | | | | K409V | Y407V | |
| 0% Combination 629 | WT | T366W/L368A/ | E356K/D399K/ | E345R/K360E/ | T394F/F405A | |
| | | Y407V | K409E/K439E | D399M/Y407A/ | | |
| | | | | K409V | | |
| 0% Combination 630 | WT | T366W/L368A/ | E356K/D399K/ | E345R/K360E/ | K392L/T394W/ | |
| | | Y407V | K409E/K439E | D399M/Y407A/ | F405A/Y407V | |
| | | | | K409V | | |
| 0% Combination 631 | WT | T366W/L368A/ | E356K/D399K/ | E345R/K360E/ | T350V/L351Y/ | |
| | | Y407V | K409E/K439E | D399M/Y407A/ | T366L/K392L/ | |
| | | | | K409V | T394W/F405A/ | |
| | | | | | Y407V | |
| 0% Combination 632 | WT | T366W/L368A/ | Y349C/E357C/ | E356K/K392D/ | E345R/K360E/ | |
| | | Y407V | K392D/D399K | D399K/K439E | D399M/Y407A/ | |
| | | | | | K409V | |
| 0% Combination 633 | WT | T366W/L368A/ | Y349C/E357C/ | E356K/K392D/ | T394F/F405A | |
| | | Y407V | K392D/D399K | D399K/K439E | | |
| 0% Combination 634 | WT | T366W/L368A/ | Y349C/E357C/ | E356K/K392D/ | K392L/T394W/ | |
| | | Y407V | K392D/D399K | D399K/K439E | F405A/Y407V | |
| 0% Combination 635 | WT | T366W/L368A/ | Y349C/E357C/ | E356K/K392D/ | T350V/L351Y/ | |
| | | Y407V | K392D/D399K | D399K/K439E | T366L/K392L/ | |
| | | | | | T394W/F405A/ | |
| | | | | | Y407V | |
| 0% Combination 636 | WT | T366W/L368A/ | Y349C/E357C/ | E356K/K392D/ | E345R/K360E/ | |
| | | Y407V | K392D/D399K | D399K/K409D | D399M/Y407A/ | |
| | | | | | K409V | |
| 0% Combination 637 | WT | T366W/L368A/ | Y349C/E357C/ | E356K/K392D/ | T394F/F405A | |
| | | Y407V | K392D/D399K | D399K/K409D | | |
| 0% Combination 638 | WT | T366W/L368A/ | Y349C/E357C/ | E356K/K392D/ | K392L/T394W/ | |
| | | Y407V | K392D/D399K | D399K/K409D | F405A/Y407V | |
| 0% Combination 639 | WT | T366W/L368A/ | Y349C/E357C/ | E356K/K392D/ | T350V/L351Y/ | |
| | | Y407V | K392D/D399K | D399K/K409D | T366L/K392L/ | |
| | | | | | T394W/F405A/ | |
| | | | | | Y407V | |
| 0% Combination 640 | WT | T366W/L368A/ | Y349C/E357C/ | E345R/K360E/ | T394F/F405A | |
| | | Y407V | K392D/D399K | D399M/Y407A/ | | |
| | | | | K409V | | |
| 0% Combination 641 | WT | T366W/L368A/ | Y349C/E357C/ | E345R/K360E/ | K392L/T394W/ | |
| | | Y407V | K392D/D399K | D399M/Y407A/ | F405A/Y407V | |
| | | | | K409V | | |
| 0% Combination 642 | WT | T366W/L368A/ | Y349C/E357C/ | E345R/K360E/ | T350V/L351Y/ | |
| | | Y407V | K392D/D399K | D399M/Y407A/ | T366L/K392L/ | |
| | | | | K409V | T394W/F405A/ | |
| | | | | | Y407V | |
| 0% Combination 643 | WT | T366W/L368A/ | E345R/Q347R/ | E356K/K392D/ | T394F/F405A | |
| | | Y407V | K360D/T366V/ | D399K/K439E | | |
| | | | D399M/Y407A/ | | | |
| | | | K409V | | | |
| 0% Combination 644 | WT | T366W/L368A/ | E345R/Q347R/ | E356K/K392D/ | K392L/T394W/ | |
| | | Y407V | K360D/T366V/ | D399K/K439E | F405A/Y407V | |
| | | | D399M/Y407A/ | | | |
| | | | K409V | | | |
| 0% Combination 645 | WT | T366W/L368A/ | E345R/Q347R/ | E356K/K392D/ | T350V/L351Y/ | |
| | | Y407V | K360D/T366V/ | D399K/K439E | T366L/K392L/ | |
| | | | D399M/Y407A/ | | T394W/F405A/ | |
| | | | K409V | | Y407V | |
| 0% Combination 646 | WT | T366W/L368A/ | E345R/Q347R/ | K392D/D399K/ | T394F/F405A | |
| | | Y407V | K360D/T366V/ | K409D | | |
| | | | D399M/Y407A/ | | | |
| | | | K409V | | | |
| 0% Combination 647 | WT | T366W/L368A/ | E345R/Q347R/ | K392D/D399K/ | K392L/T394W/ | |
| | | Y407V | K360D/T366V/ | K409D | F405A/Y407V | |
| | | | D399M/Y407A/ | | | |
| | | | K409V | | | |
| 0% Combination 648 | WT | T366W/L368A/ | E345R/Q347R/ | K392D/D399K/ | T350V/L351Y/ | |
| | | Y407V | K360D/T366V/ | K409D | T366L/K392L/ | |
| | | | D399M/Y407A/ | | T394W/F405A/ | |
| | | | K409V | | Y407V | |
| 0% Combination 649 | WT | T366W/L368A/ | E345R/Q347R/ | E356K/K392D/ | T394F/F405A | |
| | | Y407V | K360D/T366V/ | D399K/K409D | | |
| | | | D399M/Y407A/ | | | |
| | | | K409V | | | |
| 0% Combination 650 | WT | T366W/L368A/ | E345R/Q347R/ | E356K/K392D/ | K392L/T394W/ | |
| | | Y407V | K360D/T366V/ | D399K/K409D | F405A/Y407V | |
| | | | D399M/Y407A/ | | | |
| | | | K409V | | | |
| 0% Combination 651 | WT | T366W/L368A/ | E345R/Q347R/ | E356K/K392D/ | T350V/L351Y/ | |
| | | Y407V | K360D/T366V/ | D399K/K409D | T366L/K392L/ | |
| | | | D399M/Y407A/ | | T394W/F405A/ | |
| | | | K409V | | Y407V | |
| 0% Combination 652 | WT | T366W/L368A/ | E356K/K392D/ | E345R/K360E/ | T394F/F405A | |
| | | Y407V | D399K/K439E | D399M/Y407A/ | | |
| | | | | K409V | | |
| 0% Combination 653 | WT | T366W/L368A/ | E356K/K392D/ | E345R/K360E/ | K392L/T394W/ | |
| | | Y407V | D399K/K439E | D399M/Y407A/ | F405A/Y407V | |
| | | | | K409V | | |
| 0% Combination 654 | WT | T366W/L368A/ | E356K/K392D/ | E345R/K360E/ | T350V/L351Y/ | |
| | | Y407V | D399K/K439E | D399M/Y407A/ | T366L/K392L/ | |
| | | | | K409V | T394W/F405A/ | |
| | | | | | Y407V | |
| 0% Combination 655 | WT | T366W/L368A/ | E356K/K392D/ | E345R/K360E/ | T394F/F405A | |
| | | Y407V | D399K/K409D | D399M/Y407A/ | | |
| | | | | K409V | | |
| 0% Combination 656 | WT | T366W/L368A/ | E356K/K392D/ | E345R/K360E/ | K392L/T394W/ | |
| | | Y407V | D399K/K409D | D399M/Y407A/ | F405A/Y407V | |
| | | | | K409V | | |
| 0% Combination 657 | WT | T366W/L368A/ | E356K/K392D/ | E345R/K360E/ | T350V/L351Y/ | |
| | | Y407V | D399K/K409D | D399M/Y407A/ | T366L/K392L/ | |
| | | | | K409V | T394W/F405A/ | |
| | | | | | Y407V | |
| 0% Combination 658 | WT | Q347R/K360E/ | E356K/D399K/ | Y349C/E357C/ | T394F/F405A | |
| | | D399V/F405T/ | K409E/K439E | K392D/D399K | | |
| | | K409W | | | | |
| 0% Combination 659 | WT | Q347R/K360E/ | E356K/D399K/ | Y349C/E357C/ | K392L/T394W/ | |
| | | D399V/F405T/ | K409E/K439E | K392D/D399K | F405A/Y407V | |
| | | K409W | | | | |
| 0% Combination 660 | WT | Q347R/K360E/ | E356K/D399K/ | Y349C/E357C/ | T350V/L351Y/ | |
| | | D399V/F405T/ | K409E/K439E | K392D/D399K | T366L/K392L/ | |
| | | K409W | | | T394W/F405A/ | |
| | | | | | Y407V | |
| 0% Combination 661 | WT | Q347R/K360E/ | E356K/D399K/ | Q347E/S354Y/ | T394F/F405A | |
| | | D399V/F405T/ | K409E/K439E | T366Y/Y407T | | |
| | | K409W | | | | |
| 0% Combination 662 | WT | Q347R/K360E/ | E356K/D399K/ | Q347E/S354Y/ | K392L/T394W/ | |
| | | D399V/F405T/ | K409E/K439E | T366Y/Y407T | F405A/Y407V | |
| | | K409W | | | | |
| 0% Combination 663 | WT | Q347R/K360E/ | E356K/D399K/ | Q347E/S354Y/ | T350V/L351Y/ | |
| | | D399V/F405T/ | K409E/K439E | T366Y/Y407T | T366L/K392L/ | |
| | | K409W | | | T394W/F405A/ | |
| | | | | | Y407V | |
| 0% Combination 664 | WT | Q347R/K360E/ | Y349C/E357C/ | E356K/K392D/ | T394F/F405A | |
| | | D399V/F405T/ | K392D/D399K | D399K/K439E | | |
| | | K409W | | | | |
| 0% Combination 665 | WT | Q347R/K360E/ | Y349C/E357C/ | E356K/K392D/ | K392L/T394W/ | |
| | | D399V/F405T/ | K392D/D399K | D399K/K439E | F405A/Y407V | |
| | | K409W | | | | |
| 0% Combination 666 | WT | Q347R/K360E/ | Y349C/E357C/ | E356K/K392D/ | T350V/L351Y/ | |
| | | D399V/F405T/ | K392D/D399K | D399K/K439E | T366L/K392L/ | |
| | | K409W | | | T394W/F405A/ | |
| | | | | | Y407V | |
| 0% Combination 667 | WT | Q347R/K360E/ | Y349C/E357C/ | E356K/K392D/ | T394F/F405A | |
| | | D399V/F405T/ | K392D/D399K | D399K/K409D | | |
| | | K409W | | | | |
| 0% Combination 668 | WT | Q347R/K360E/ | Y349C/E357C/ | E356K/K392D/ | K392L/T394W/ | |
| | | D399V/F405T/ | K392D/D399K | D399K/K409D | F405A/Y407V | |
| | | K409W | | | | |
| 0% Combination 669 | WT | Q347R/K360E/ | Y349C/E357C/ | E356K/K392D/ | T350V/L351Y/ | |
| | | D399V/F405T/ | K392D/D399K | D399K/K409D | T366L/K392L/ | |
| | | K409W | | | T394W/F405A/ | |
| | | | | | Y407V | |
| 0% Combination 670 | WT | Q347R/K360E/ | Q347E/S354Y/ | E356K/K392D/ | T394F/F405A | |
| | | D399V/F405T/ | T366Y/Y407T | D399K/K439E | | |
| | | K409W | | | | |
| 0% Combination 671 | WT | Q347R/K360E/ | Q347E/S354Y/ | E356K/K392D/ | K392L/T394W/ | |
| | | D399V/F405T/ | T366Y/Y407T | D399K/K439E | F405A/Y407V | |
| | | K409W | | | | |
| 0% Combination 672 | WT | Q347R/K360E/ | Q347E/S354Y/ | E356K/K392D/ | T350V/L351Y/ | |
| | | D399V/F405T/ | T366Y/Y407T | D399K/K439E | T366L/K392L/ | |
| | | K409W | | | T394W/F405A/ | |
| | | | | | Y407V | |
| 0% Combination 673 | WT | Q347R/K360E/ | Q347E/S354Y/ | K392D/D399K/ | T394F/F405A | |
| | | D399V/F405T/ | T366Y/Y407T | K409D | | |
| | | K409W | | | | |
| 0% Combination 674 | WT | Q347R/K360E/ | Q347E/S354Y/ | K392D/D399K/ | K392L/T394W/ | |
| | | D399V/F405T/ | T366Y/Y407T | K409D | F405A/Y407V | |
| | | K409W | | | | |
| 0% Combination 675 | WT | Q347R/K360E/ | Q347E/S354Y/ | K392D/D399K/ | T350V/L351Y/ | |
| | | D399V/F405T/ | T366Y/Y407T | K409D | T366L/K392L/ | |
| | | K409W | | | T394W/F405A/ | |
| | | | | | Y407V | |
| 0% Combination 676 | WT | Q347R/K360E/ | Q347E/S354Y/ | E356K/K392D/ | T394F/F405A | |
| | | D399V/F405T/ | T366Y/Y407T | D399K/K409D | | |
| | | K409W | | | | |
| 0% Combination 677 | WT | Q347R/K360E/ | Q347E/S354Y/ | E356K/K392D/ | K392L/T394W/ | |
| | | D399V/F405T/ | T366Y/Y407T | D399K/K409D | F405A/Y407V | |
| | | K409W | | | | |
| 0% Combination 678 | WT | Q347R/K360E/ | Q347E/S354Y/ | E356K/K392D/ | T350V/L351Y/ | |
| | | D399V/F405T/ | T366Y/Y407T | D399K/K409D | T366L/K392L/ | |
| | | K409W | | | T394W/F405A/ | |
| | | | | | Y407V | |
| 0% Combination 679 | WT | E356K/D399K/ | Y349C/E357C/ | E345R/K360E/ | T394F/F405A | |
| | | K409E/K439E | K392D/D399K | D399M/Y407A/ | | |
| | | | | K409V | | |
| 0% Combination 680 | WT | E356K/D399K/ | Y349C/E357C/ | E345R/K360E/ | K392L/T394W/ | |
| | | K409E/K439E | K392D/D399K | D399M/Y407A/ | F405A/Y407V | |
| | | | | K409V | | |
| 0% Combination 681 | WT | E356K/D399K/ | Y349C/E357C/ | E345R/K360E/ | T350V/L351Y/ | |
| | | K409E/K439E | K392D/D399K | D399M/Y407A/ | T366L/K392L/ | |
| | | | | K409V | T394W/F405A/ | |
| | | | | | Y407V | |
| 0% Combination 682 | WT | E356K/D399K/ | Q347E/S354Y/ | E345R/K360E/ | T394F/F405A | |
| | | K409E/K439E | T366Y/Y407T | D399M/Y407A/ | | |
| | | | | K409V | | |
| 0% Combination 683 | WT | E356K/D399K/ | Q347E/S354Y/ | E345R/K360E/ | K392L/T394W/ | |
| | | K409E/K439E | T366Y/Y407T | D399M/Y407A/ | F405A/Y407V | |
| | | | | K409V | | |
| 0% Combination 684 | WT | E356K/D399K/ | Q347E/S354Y/ | E345R/K360E/ | T350V/L351Y/ | |
| | | K409E/K439E | T366Y/Y407T | D399M/Y407A/ | T366L/K392L/ | |
| | | | | K409V | T394W/F405A/ | |
| | | | | | Y407V | |
| 0% Combination 685 | WT | Y349C/E357C/ | E356K/K392D/ | E345R/K360E/ | T394F/F405A | |
| | | K392D/D399K | D399K/K439E | D399M/Y407A/ | | |
| | | | | K409V | | |
| 0% Combination 686 | WT | Y349C/E357C/ | E356K/K392D/ | E345R/K360E/ | K392L/T394W/ | |
| | | K392D/D399K | D399K/K439E | D399M/Y407A/ | F405A/Y407V | |
| | | | | K409V | | |
| 0% Combination 687 | WT | Y349C/E357C/ | E356K/K392D/ | E345R/K360E/ | T350V/L351Y/ | |
| | | K392D/D399K | D399K/K439E | D399M/Y407A/ | T366L/K392L/ | |
| | | | | K409V | T394W/F405A/ | |
| | | | | | Y407V | |
| 0% Combination 688 | WT | Y349C/E357C/ | E356K/K392D/ | E345R/K360E/ | T394F/F405A | |
| | | K392D/D399K | D399K/K409D | D399M/Y407A/ | | |
| | | | | K409V | | |
| 0% Combination 689 | WT | Y349C/E357C/ | E356K/K392D/ | E345R/K360E/ | K392L/T394W/ | |
| | | K392D/D399K | D399K/K409D | D399M/Y407A/ | F405A/Y407V | |
| | | | | K409V | | |
| 0% Combination 690 | WT | Y349C/E357C/ | E356K/K392D/ | E345R/K360E/ | T350V/L351Y/ | |
| | | K392D/D399K | D399K/K409D | D399M/Y407A/ | T366L/K392L/ | |
| | | | | K409V | T394W/F405A/ | |
| | | | | | Y407V | |
| 0% Combination 691 | WT | Q347E/S354Y/ | E356K/K392D/ | E345R/K360E/ | T394F/F405A | |
| | | T366Y/Y407T | D399K/K439E | D399M/Y407A/ | | |
| | | | | K409V | | |
| 0% Combination 692 | WT | Q347E/S354Y/ | E356K/K392D/ | E345R/K360E/ | K392L/T394W/ | |
| | | T366Y/Y407T | D399K/K439E | D399M/Y407A/ | F405A/Y407V | |
| | | | | K409V | | |
| 0% Combination 693 | WT | Q347E/S354Y/ | E356K/K392D/ | E345R/K360E/ | T350V/L351Y/ | |
| | | T366Y/Y407T | D399K/K439E | D399M/Y407A/ | T366L/K392L/ | |
| | | | | K409V | T394W/F405A/ | |
| | | | | | Y407V | |
| 0% Combination 694 | WT | Q347E/S354Y/ | E356K/K392D/ | E345R/K360E/ | T394F/F405A | |
| | | T366Y/Y407T | D399K/K409D | D399M/Y407A/ | | |
| | | | | K409V | | |
| 0% Combination 695 | WT | Q347E/S354Y/ | E356K/K392D/ | E345R/K360E/ | K392L/T394W/ | |
| | | T366Y/Y407T | D399K/K409D | D399M/Y407A/ | F405A/Y407V | |
| | | | | K409V | | |
| 0% Combination 696 | WT | Q347E/S354Y/ | E356K/K392D/ | E345R/K360E/ | T350V/L351Y/ | |
| | | T366Y/Y407T | D399K/K409D | D399M/Y407A/ | T366L/K392L/ | |
| | | | | K409V | T394W/F405A/ | |
| | | | | | Y407V | |
| 0% Combination 697 | T394W/F405A | T366W/L368A/ | Q347R/K360E/ | E356K/D399K/ | Y349C/E357C/ | |
| | | Y407V | D399V/F405T/ | K409E/K439E | K392D/D399K | |
| | | | K409W | | | |
| 0% Combination 698 | T394W/F405A | T366W/L368A/ | Q347R/K360E/ | Y349C/E357C/ | E356K/K392D/ | |
| | | Y407V | D399V/F405T/ | K392D/D399K | D399K/K439E | |
| | | | K409W | | | |
| 0% Combination 699 | T394W/F405A | T366W/L368A/ | Q347R/K360E/ | Y349C/E357C/ | E356K/K392D/ | |
| | | Y407V | D399V/F405T/ | K392D/D399K | D399K/K409D | |
| | | | K409W | | | |
| 0% Combination 700 | T394W/F405A | T366W/L368A/ | E356K/D399K/ | Y349C/E357C/ | E345R/K360E/ | |
| | | Y407V | K409E/K439E | K392D/D399K | D399M/Y407A/ | |
| | | | | | K409V | |
| 0% Combination 701 | T394W/F405A | T366W/L368A/ | Y349C/E357C/ | E356K/K392D/ | E345R/K360E/ | |
| | | Y407V | K392D/D399K | D399K/K439E | D399M/Y407A/ | |
| | | | | | K409V | |
| 0% Combination 702 | T394W/F405A | T366W/L368A/ | Y349C/E357C/ | E356K/K392D/ | E345R/K360E/ | |
| | | Y407V | K392D/D399K | D399K/K409D | D399M/Y407A/ | |
| | | | | | K409V | |
| 0% Combination 703 | T366W/L368A/ | Q347R/K360E/ | E356K/D399K/ | Y349C/E357C/ | T394F/F405A | |
| | Y407V | D399V/F405T/ | K409E/K439E | K392D/D399K | | |
| | | K409W | | | | |
| 0% Combination 704 | T366W/L368A/ | Q347R/K360E/ | E356K/D399K/ | Y349C/E357C/ | K392L/T394W/ | |
| | Y407V | D399V/F405T/ | K409E/K439E | K392D/D399K | F405A/Y407V | |
| | | K409W | | | | |
| 0% Combination 705 | T366W/L368A/ | Q347R/K360E/ | E356K/D399K/ | Y349C/E357C/ | T350V/L351Y/ | |
| | Y407V | D399V/F405T/ | K409E/K439E | K392D/D399K | T366L/K392L/ | |
| | | K409W | | | T394W/F405A/ | |
| | | | | | Y407V | |
| 0% Combination 706 | T366W/L368A/ | Q347R/K360E/ | Y349C/E357C/ | E356K/K392D/ | T394F/F405A | |
| | Y407V | D399V/F405T/ | K392D/D399K | D399K/K439E | | |
| | | K409W | | | | |
| 0% Combination 707 | T366W/L368A/ | Q347R/K360E/ | Y349C/E357C/ | E356K/K392D/ | K392L/T394W/ | |
| | Y407V | D399V/F405T/ | K392D/D399K | D399K/K439E | F405A/Y407V | |
| | | K409W | | | | |
| 0% Combination 708 | T366W/L368A/ | Q347R/K360E/ | Y349C/E357C/ | E356K/K392D/ | T350V/L351Y/ | |
| | Y407V | D399V/F405T/ | K392D/D399K | D399K/K439E | T366L/K392L/ | |
| | | K409W | | | T394W/F405A/ | |
| | | | | | Y407V | |
| 0% Combination 709 | T366W/L368A/ | Q347R/K360E/ | Y349C/E357C/ | E356K/K392D/ | T394F/F405A | |
| | Y407V | D399V/F405T/ | K392D/D399K | D399K/K409D | | |
| | | K409W | | | | |
| 0% Combination 710 | T366W/L368A/ | Q347R/K360E/ | Y349C/E357C/ | E356K/K392D/ | K392L/T394W/ | |
| | Y407V | D399V/F405T/ | K392D/D399K | D399K/K409D | F405A/Y407V | |
| | | K409W | | | | |
| 0% Combination 711 | T366W/L368A/ | Q347R/K360E/ | Y349C/E357C/ | E356K/K392D/ | T350V/L351Y/ | |
| | Y407V | D399V/F405T/ | K392D/D399K | D399K/K409D | T366L/K392L/ | |
| | | K409W | | | T394W/F405A/ | |
| | | | | | Y407V | |
| 0% Combination 712 | T366W/L368A/ | E356K/D399K/ | Y349C/E357C/ | E345R/K360E/ | T394F/F405A | |
| | Y407V | K409E/K439E | K392D/D399K | D399M/Y407A/ | | |
| | | | | K409V | | |
| 0% Combination 713 | T366W/L368A/ | E356K/D399K/ | Y349C/E357C/ | E345R/K360E/ | K392L/T394W/ | |
| | Y407V | K409E/K439E | K392D/D399K | D399M/Y407A/ | F405A/Y407V | |
| | | | | K409V | | |
| 0% Combination 714 | T366W/L368A/ | E356K/D399K/ | Y349C/E357C/ | E345R/K360E/ | T350V/L351Y/ | |
| | Y407V | K409E/K439E | K392D/D399K | D399M/Y407A/ | T366L/K392L/ | |
| | | | | K409V | T394W/F405A/ | |
| | | | | | Y407V | |
| 0% Combination 715 | T366W/L368A/ | Y349C/E357C/ | E356K/K392D/ | E345R/K360E/ | T394F/F405A | |
| | Y407V | K392D/D399K | D399K/K439E | D399M/Y407A/ | | |
| | | | | K409V | | |
| 0% Combination 716 | T366W/L368A/ | Y349C/E357C/ | E356K/K392D/ | E345R/K360E/ | K392L/T394W/ | |
| | Y407V | K392D/D399K | D399K/K439E | D399M/Y407A/ | F405A/Y407V | |
| | | | | K409V | | |
| 0% Combination 717 | T366W/L368A/ | Y349C/E357C/ | E356K/K392D/ | E345R/K360E/ | T350V/L351Y/ | |
| | Y407V | K392D/D399K | D399K/K439E | D399M/Y407A/ | T366L/K392L/ | |
| | | | | K409V | T394W/F405A/ | |
| | | | | | Y407V | |
| 0% Combination 718 | T366W/L368A/ | Y349C/E357C/ | E356K/K392D/ | E345R/K360E/ | T394F/F405A | |
| | Y407V | K392D/D399K | D399K/K409D | D399M/Y407A/ | | |
| | | | | K409V | | |
| 0% Combination 719 | T366W/L368A/ | Y349C/E357C/ | E356K/K392D/ | E345R/K360E/ | K392L/T394W/ | |
| | Y407V | K392D/D399K | D399K/K409D | D399M/Y407A/ | F405A/Y407V | |
| | | | | K409V | | |
| 0% Combination 720 | T366W/L368A/ | Y349C/E357C/ | E356K/K392D/ | E345R/K360E/ | T350V/L351Y/ | |
| | Y407V | K392D/D399K | D399K/K409D | D399M/Y407A/ | T366L/K392L/ | |
| | | | | K409V | T394W/F405A/ | |
| | | | | | Y407V | |
| 0% Combination 721 | WT | T394W/F405A | T366W/L368A/ | Q347R/K360E/ | E356K/D399K/ | Y349C/E357C/ |
| | | | Y407V | D399V/F405T/ | K409E/K439E | K392D/D399K |
| | | | | K409W | | |
| 0% Combination 722 | WT | T394W/F405A | T366W/L368A/ | Q347R/K360E/ | Y349C/E357C/ | E356K/K392D/ |
| | | | Y407V | D399V/F405T/ | K392D/D399K | D399K/K439E |
| | | | | K409W | | |
| 0% Combination 723 | WT | T394W/F405A | T366W/L368A/ | Q347R/K360E/ | Y349C/E357C/ | E356K/K392D/ |
| | | | Y407V | D399V/F405T/ | K392D/D399K | D399K/K409D |
| | | | | K409W | | |
| 0% Combination 724 | WT | T394W/F405A | T366W/L368A/ | E356K/D399K/ | Y349C/E357C/ | E345R/K360E/ |
| | | | Y407V | K409E/K439E | K392D/D399K | D399M/Y407A/ |
| | | | | | | K409V |
| 0% Combination 725 | WT | T394W/F405A | T366W/L368A/ | Y349C/E357C/ | E356K/K392D/ | E345R/K360E/ |
| | | | Y407V | K392D/D399K | D399K/K439E | D399M/Y407A/ |
| | | | | | | K409V |
| 0% Combination 726 | WT | T394W/F405A | T366W/L368A/ | Y349C/E357C/ | E356K/K392D/ | E345R/K360E/ |
| | | | Y407V | K392D/D399K | D399K/K409D | D399M/Y407A/ |
| | | | | | | K409V |
| 0% Combination 727 | WT | T366W/L368A/ | Q347R/K360E/ | E356K/D399K/ | Y349C/E357C/ | T394F/F405A |
| | | Y407V | D399V/F405T/ | K409E/K439E | K392D/D399K | |
| | | | K409W | | | |
| 0% Combination 728 | WT | T366W/L368A/ | Q347R/K360E/ | E356K/D399K/ | Y349C/E357C/ | K392L/T394W/ |
| | | Y407V | D399V/F405T/ | K409E/K439E | K392D/D399K | F405A/Y407V |
| | | | K409W | | | |
| 0% Combination 729 | WT | T366W/L368A/ | Q347R/K360E/ | E356K/D399K/ | Y349C/E357C/ | T350V/L351Y/ |
| | | Y407V | D399V/F405T/ | K409E/K439E | K392D/D399K | T366L/K392L/ |
| | | | K409W | | | T394W/F405A/ |
| | | | | | | Y407V |
| 0% Combination 730 | WT | T366W/L368A/ | Q347R/K360E/ | Y349C/E357C/ | E356K/K392D/ | T394F/F405A |
| | | Y407V | D399V/F405T/ | K392D/D399K | D399K/K439E | |
| | | | K409W | | | |
| 0% Combination 731 | WT | T366W/L368A/ | Q347R/K360E/ | Y349C/E357C/ | E356K/K392D/ | K392L/T394W/ |
| | | Y407V | D399V/F405T/ | K392D/D399K | D399K/K439E | F405A/Y407V |
| | | | K409W | | | |
| 0% Combination 732 | WT | T366W/L368A/ | Q347R/K360E/ | Y349C/E357C/ | E356K/K392D/ | T350V/L351Y/ |
| | | Y407V | D399V/F405T/ | K392D/D399K | D399K/K439E | T366L/K392L/ |
| | | | K409W | | | T394W/F405A/ |
| | | | | | | Y407V |
| 0% Combination 733 | WT | T366W/L368A/ | Q347R/K360E/ | Y349C/E357C/ | E356K/K392D/ | T394F/F405A |
| | | Y407V | D399V/F405T/ | K392D/D399K | D399K/K409D | |
| | | | K409W | | | |
| 0% Combination 734 | WT | T366W/L368A/ | Q347R/K360E/ | Y349C/E357C/ | E356K/K392D/ | K392L/T394W/ |
| | | Y407V | D399V/F405T/ | K392D/D399K | D399K/K409D | F405A/Y407V |
| | | | K409W | | | |
| 0% Combination 735 | WT | T366W/L368A/ | Q347R/K360E/ | Y349C/E357C/ | E356K/K392D/ | T350V/L351Y/ |
| | | Y407V | D399V/F405T/ | K392D/D399K | D399K/K409D | T366L/K392L/ |
| | | | K409W | | | T394W/F405A/ |
| | | | | | | Y407V |
| 0% Combination 736 | WT | T366W/L368A/ | E356K/D399K/ | Y349C/E357C/ | E345R/K360E/ | T394F/F405A |
| | | Y407V | K409E/K439E | K392D/D399K | D399M/Y407A/ | |
| | | | | | K409V | |
| 0% Combination 737 | WT | T366W/L368A/ | E356K/D399K/ | Y349C/E357C/ | E345R/K360E/ | K392L/T394W/ |
| | | Y407V | K409E/K439E | K392D/D399K | D399M/Y407A/ | F405A/Y407V |
| | | | | | K409V | |
| 0% Combination 738 | WT | T366W/L368A/ | E356K/D399K/ | Y349C/E357C/ | E345R/K360E/ | T350V/L351Y/ |
| | | Y407V | K409E/K439E | K392D/D399K | D399M/Y407A/ | T366L/K392L/ |
| | | | | | K409V | T394W/F405A/ |
| | | | | | | Y407V |
| 0% Combination 739 | WT | T366W/L368A/ | Y349C/E357C/ | E356K/K392D/ | E345R/K360E/ | T394F/F405A |
| | | Y407V | K392D/D399K | D399K/K439E | D399M/Y407A/ | |
| | | | | | K409V | |
| 0% Combination 740 | WT | T366W/L368A/ | Y349C/E357C/ | E356K/K392D/ | E345R/K360E/ | K392L/T394W/ |
| | | Y407V | K392D/D399K | D399K/K439E | D399M/Y407A/ | F405A/Y407V |
| | | | | | K409V | |
| 0% Combination 741 | WT | T366W/L368A/ | Y349C/E357C/ | E356K/K392D/ | E345R/K360E/ | T350V/L351Y/ |
| | | Y407V | K392D/D399K | D399K/K439E | D399M/Y407A/ | T366L/K392L/ |
| | | | | | K409V | T394W/F405A/ |
| | | | | | | Y407V |
| 0% Combination 742 | WT | T366W/L368A/ | Y349C/E357C/ | E356K/K392D/ | E345R/K360E/ | T394F/F405A |
| | | Y407V | K392D/D399K | D399K/K409D | D399M/Y407A/ | |
| | | | | | K409V | |
| 0% Combination 743 | WT | T366W/L368A/ | Y349C/E357C/ | E356K/K392D/ | E345R/K360E/ | K392L/T394W/ |
| | | Y407V | K392D/D399K | D399K/K409D | D399M/Y407A/ | F405A/Y407V |
| | | | | | K409V | |
| 0% Combination 744 | WT | T366W/L368A/ | Y349C/E357C/ | E356K/K392D/ | E345R/K360E/ | T350V/L351Y/ |
| | | Y407V | K392D/D399K | D399K/K409D | D399M/Y407A/ | T366L/K392L/ |
| | | | | | K409V | T394W/F405A/ |
| | | | | | | Y407V |

### [Example 6] Confirmation of Coexpression of Multiple Antibodies by Ion Exchange Chromatography (IEC)

Example 5 identified pairs that allow coexpression of two or more antibodies. In order to confirm whether they can actually achieve promotion of homodimer formation between multiple IgG antibodies, Expi293F was allowed to express multiple modified IgG antibodies. For expression, a plasmid encoding the heavy chain and a plasmid encoding the light chain were used at a mass ratio of 1:1 for transfection. When multiple heavy chains were used for expression, they were used such that the mass ratio of each heavy chain was equal. Each antibody after Protein A purification was analyzed by ion exchange chromatography (IEC). Specifically, the produced antibody was analyzed by cation exchange chromatography (CIEX) using Alliance system (Waters). A two-solvent gradient method was performed using YMC-BioPro SP-F 5 μm 4.6×100 (YMC) as an analysis column, CX-1 pH Gradient Buffer A, pH 5.6 (Thermo) as mobile phase A, and CX-1 pH Gradient Buffer B, pH 10.2 (Thermo) as mobile phase B. Samples were detected by absorption at a wavelength of 280 nm (denoted as AU on the vertical axes in figures). Data was analyzed using Empower3 (Waters) to calculate the ratio of each detected peak.

The modifications used in antibody expression and the corresponding SEQ ID NOs are shown in Table 8 (the non-introduction of modifications is denoted as “−”). In expression, the heavy chains of four different antibodies with different isoelectric points (pl) were used and, to simplify analysis, a common light chain (SEQ ID NO: 61) was used. The antibodies prior to modification and the individual modified antibodies were also analyzed for assignment of the peaks detected when the multiple heavy chains were expressed. Analysis results are shown in FIGS. 3-1 to 3-47 along with the assignment and area ratio of each peak. In these figures, peak assignments are indicated using abbreviations that feature heavy chain association. The abbreviations and the correspondences between the template heavy chain sequences and the modifications are shown in Table 9 (the non-introduction of modifications is denoted as “−”). For example, the antibody with homodimerized identical heavy chains of SEQ ID NO: 1 and the common light chain is denoted as “aa”. All combinations implemented were found to have a strong heavy-chain homomeric association-promoting ability. Similarly, the combinations shown in Tables 6 and 7 are also considered to exhibit a heavy-chain homomeric association ability.

TABLE 8
Correspondences between SEQ ID NOs and amino acid modifications in the samples
used for confirming controlled heavy-chain association of multiple antibodies

| | Heavy | | Heavy | | Heavy | | Heavy | | |
|---|---|---|---|---|---|---|---|---|---|
| | chain | | chain | | chain | | chain | Modifications that | Light |
| | 1: | Modifications that | 2: | Modifications that | 3: | Modifications that | 4: | promote | chain: |
| | SEQ | promote formation | SEQ | promote formation | SEQ | promote formation | SEQ | formation of | SEQ |
| Sample | ID | of heavy chain | ID | of heavy chain | ID | of heavy chain | ID | heavy chain | ID |
| No. | NO | homodimers | NO | homodimers | NO | homodimers | NO | homodimers | NO |
| 122 | 1 | — | | | | | | | 61 |
| 123 | 78 | — | | | | | | | 61 |
| 124 | 79 | — | | | | | | | 61 |
| 125 | 80 | — | | | | | | | 61 |
| 126 | 1 | — | 78 | — | | | | | 61 |
| 127 | 1 | — | 79 | — | | | | | 61 |
| 128 | 1 | — | 80 | — | | | | | 61 |
| 129 | 78 | — | 79 | — | | | | | 61 |
| 130 | 78 | — | 80 | — | | | | | 61 |
| 131 | 79 | — | 80 | — | | | | | 61 |
| 132 | 1 | — | 78 | — | 79 | — | | | 61 |
| 133 | 78 | — | 79 | — | 80 | — | | | 61 |
| 134 | 79 | — | 80 | — | 1 | — | | | 61 |
| 135 | 1 | — | 78 | — | 79 | — | 80 | — | 61 |
| 136 | 19 | T394W/F405A | | | | | | | 61 |
| 137 | 81 | T366W/L368A/ | | | | | | | 61 |
| | | Y407V | | | | | | | |
| 138 | 82 | Q347R/K360E/ | | | | | | | 61 |
| | | D399V/F405T/ | | | | | | | |
| | | K409W | | | | | | | |
| 139 | 83 | E356K/D399K/ | | | | | | | 61 |
| | | K409E/K439E | | | | | | | |
| 140 | 19 | T394W/F405A | 81 | T366W/L368A/ | | | | | 61 |
| | | | | Y407V | | | | | |
| 141 | 19 | T394W/F405A | 82 | Q347R/K360E/ | | | | | 61 |
| | | | | D399V/F405T/ | | | | | |
| | | | | K409W | | | | | |
| 142 | 19 | T394W/F405A | 83 | E356K/D399K/ | | | | | 61 |
| | | | | K409E/K439E | | | | | |
| 143 | 81 | T366W/L368A/ | 82 | Q347R/K360E/ | | | | | 61 |
| | | Y407V | | D399V/F405T/ | | | | | |
| | | | | K409W | | | | | |
| 144 | 81 | T366W/L368A/ | 83 | E356K/K392D/ | | | | | 61 |
| | | Y407V | | D399K/K439E | | | | | |
| 145 | 82 | Q347R/K360E/ | 83 | E356K/D399K/ | | | | | 61 |
| | | D399V/F405T/ | | K409E/K439E | | | | | |
| | | K409W | | | | | | | |
| 146 | 19 | T394W/F405A | 81 | T366W/L368A/ | 82 | Q347R/K360E/ | | | 61 |
| | | | | Y407V | | D399V/F405T/ | | | |
| | | | | | | K409W | | | |
| 147 | 81 | T366W/L368A/ | 82 | Q347R/K360E/ | 83 | E356K/K392D/ | | | 61 |
| | | Y407V | | D399V/F405T/ | | D399K/K439E | | | |
| | | | | K409W | | | | | |
| 148 | 82 | Q347R/K360E/ | 83 | E356K/D399K/ | 19 | T394W/F405A | | | 61 |
| | | D399V/F405T/ | | K409E/K439E | | | | | |
| | | K409W | | | | | | | |
| 149 | 19 | T394W/F405A | 81 | T366W/L368A/ | 82 | Q347R/K360E/ | 83 | E356K/D399K/ | 61 |
| | | | | Y407V | | D399V/F405T/ | | K409E/K439E | |
| | | | | | | K409W | | | |
| 150 | 84 | E356K/K392D/ | | | | | | | 61 |
| | | D399K/K439E | | | | | | | |
| 151 | 19 | T394W/F405A | 84 | E356K/K392D/ | | | | | 61 |
| | | | | D399K/K439E | | | | | |
| 152 | 81 | T366W/L368A/ | 84 | E356K/K392D/ | | | | | 61 |
| | | Y407V | | D399K/K439E | | | | | |
| 153 | 82 | Q347R/K360E/ | 84 | E356K/K392D/ | | | | | 61 |
| | | D399V/F405T/ | | D399K/K439E | | | | | |
| | | K409W | | | | | | | |
| 154 | 81 | T366W/L368A/ | 82 | Q347R/K360E/ | 84 | E356K/K392D/ | | | 61 |
| | | Y407V | | D399V/F405T/ | | D399K/K439E | | | |
| | | | | K409W | | | | | |
| 155 | 82 | Q347R/K360E/ | 84 | E356K/K392D/ | 19 | T394W/F405A | | | 61 |
| | | D399V/F405T/ | | D399K/K439E | | | | | |
| | | K409W | | | | | | | |
| 156 | 19 | T394W/F405A | 81 | T366W/L368A/ | 82 | Q347R/K360E/ | 84 | E356K/K392D/ | 61 |
| | | | | Y407V | | D399V/F405T/ | | D399K/K439E | |
| | | | | | | K409W | | | |
| 157 | 85 | E345R/K360E/ | | | | | | | 61 |
| | | D399M/Y407A/ | | | | | | | |
| | | K409V | | | | | | | |
| 158 | 19 | T394W/F405A | 85 | E345R/K360E/ | | | | | 61 |
| | | | | D399M/Y407A/ | | | | | |
| | | | | K409V | | | | | |
| 159 | 81 | T366W/L368A/ | 85 | E345R/K360E/ | | | | | 61 |
| | | Y407V | | D399M/Y407A/ | | | | | |
| | | | | K409V | | | | | |
| 160 | 85 | E345R/K360E/ | 83 | E356K/D399K/ | | | | | 61 |
| | | D399M/Y407A/ | | K409E/K439E | | | | | |
| | | K409V | | | | | | | |
| 161 | 19 | T394W/F405A | 81 | T366W/L368A/ | 85 | E345R/K360E/ | | | 61 |
| | | | | Y407V | | D399M/Y407A/ | | | |
| | | | | | | K409V | | | |
| 162 | 81 | T366W/L368A/ | 85 | E345R/K360E/ | 83 | E356K/D399K/ | | | 61 |
| | | Y407V | | D399M/Y407A/ | | K409E/K439E | | | |
| | | | | K409V | | | | | |
| 163 | 85 | E345R/K360E/ | 83 | E356K/D399K/ | 19 | T394W/F405A | | | 61 |
| | | D399M/Y407A/ | | K409E/K439E | | | | | |
| | | K409V | | | | | | | |
| 164 | 19 | T394W/F405A | 81 | T366W/L368A/ | 85 | E345R/K360E/ | 83 | E356K/D399K/ | 61 |
| | | | | Y407V | | D399M/Y407A/ | | K409E/K439E | |
| | | | | | | K409V | | | |
| 165 | 85 | E345R/K360E/ | 84 | E356K/K392D/ | | | | | 61 |
| | | D399M/Y407A/ | | D399K/K439E | | | | | |
| | | K409V | | | | | | | |
| 166 | 81 | T366W/L368A/ | 85 | E345R/K360E/ | 84 | E356K/K392D/ | | | 61 |
| | | Y407V | | D399M/Y407A/ | | D399K/K439E | | | |
| | | | | K409V | | | | | |
| 167 | 85 | E345R/K360E/ | 84 | E356K/K392D/ | 19 | T394W/F405A | | | 61 |
| | | D399M/Y407A/ | | D399K/K439E | | | | | |
| | | K409V | | | | | | | |
| 168 | 19 | T394W/F405A | 81 | T366W/L368A/ | 85 | E345R/K360E/ | 84 | E356K/K392D/ | 61 |
| | | | | Y407V | | D399M/Y407A/ | | D399K/K439E | |
| | | | | | | K409V | | | |

TABLE 9
Correspondences between template heavy chains, introduced modifications, and heavy chain abbreviations in figures and tables

| Template | Heavy | | | Heavy | | | Heavy | |
|---|---|---|---|---|---|---|---|---|
| heavy | chain | | | chain | | | chain | |
| chain: | abbre- | | Heavy chain | abbre- | Modifications introduced | Heavy chain | abbre- | Modifications introduced |
| SEQ ID NO | viation | Modification | SEQ ID NO | viation | into template heavy chain | SEQ ID NO | viation | into template heavy chain |
| 1 | a | — | 19 | A | T394W/F405A | | | |
| 78 | b | — | 81 | B | T366W/L368A/Y407V | | | |
| 79 | c | — | 82 | C | Q347R/K360E/D399V/ | 85 | C′ | E345R/K360E/D399M/ |
| | | | | | F405T/K409W | | | Y407A/K409V |
| 80 | d | — | 83 | D | E356K/D399K/K409E/ | 84 | D′ | E356K/K392D/D399K/ |
| | | | | | K439E | | | K439E |

### [Example 7] Evaluation of Human FcRn Binding of Antibodies with Modifications Introduced into CH3 Region

Next, the effect of the modifications for promoting heavy-chain homomeric association on Fc functions was examined.

Binding to human neonatal Fc receptor (FcRn) was evaluated using Biacore T200 (Cytiva). Evaluation was performed at 25° C. using 50 mM phosphate buffer, 150 mM NaCl, 0.05 w/v %-P20, pH6.0, as a running buffer. rProtein L (BioVision) was immobilized onto Series S CM4 (Cytiva) as a ligand-capturing molecule. An antibody solution prepared with the running buffer was allowed to interact with this CM4 sensor chip to capture about 400 RU of the antibody. The human FcRn protein used in this measurement was prepared by the method described in WO2010107110. Human FcRn was diluted to 0, 250, 500, 1000, 2000, and 4000 nM with the running buffer and allowed to bind to the captured antibody. The chip was regenerated using 10 mM Glycine-HCl (pH 1.5) and repeatedly used to capture antibodies for measurement. The FcRn-binding activity of each antibody was evaluated by calculating KD (M) using Biacore T200 Evaluation Software 3.2.1 with a steady-state model. KD values were similar in the presence and absence of the modifications in the CH3 interface. Table 10 shows the KD (M) between human FcRn and each antibody (the non-introduction of modifications is denoted as “−”).

TABLE 10
Evaluation of the human FcRn-binding activity of antibodies
with modifications introduced into the CH3 region

| Sample | Modifications that promote formation | KD value (M) |
|---|---|---|
| No. | of heavy chain homodimers | for human FcRn |
| 61 | — | 1.83E−06 |
| 80 | T394W/F405A | 1.83E−06 |
| 87 | T366W/L368A/Y407V | 1.73E−06 |
| 92 | Q347R/K360E/D399V/F405T/K409W | 1.86E−06 |
| 95 | T394F/F405A | 1.77E−06 |
| 97 | E356K/D399K/K409E/K439E | 1.74E−06 |
| 100 | Q347E/S354Y/T366Y/Y407T | 1.76E−06 |
| 102 | K392L/T394W/F405A/Y407V | 1.69E−06 |
| 109 | E356K/K392D/D399K/K439E | 1.69E−06 |
| 111 | K392D/D399K/K409D | 1.53E−06 |
| 112 | Y349C/E357C/K392D/D399K | 1.57E−06 |
| 116 | E345R/Q347R/K360D/T366V/D399M/ | 1.78E−06 |
| | Y407A/K409V | |
| 117 | E356K/K392D/D399K/K409D | 1.59E−06 |
| 119 | E345R/K360E/D399M/Y407A/K409V | 1.61E−06 |
| 121 | T350V/L351Y/T366L/K392L/T394W/ | 1.73E−06 |
| | F405A/Y407V | |

### [Example 8] Evaluation of Human Fcγ Receptor Binding of Antibodies with Modifications Introduced into the CH3 Region

The binding activity of the produced modified antibodies for each human Fcγ receptor (hereinafter denoted as FcγR) was evaluated using Biacore T200 (Cytiva). Evaluation was performed at 25° C. using 50 mM phosphate buffer, 150 mM NaCl, 0.05 w/v %-P20, pH7.4, as a running buffer. rProtein L (BioVision) was immobilized onto Series S CM4 (Cytiva) as a ligand-capturing molecule. An antibody solution prepared with the running buffer was allowed to interact with this CM4 sensor chip to capture about 500 RU of the antibody in the case of measurement for human FcγRIa, and 2000 RU in the case of measurement for the other human FcγRs. The human FcγR proteins used in this measurement were prepared by the method described in WO2022220275. The human FcγR was diluted with the running buffer to 8 nM in the case of FcγRIa or 1000 nM in the case of the other FcγRs, and allowed to bind to the captured antibody. The chip was regenerated using 10 mM Glycine-HCl (pH 1.5) and repeatedly used to capture antibodies for measurement. The binding activity of each antibody to each FcγR was evaluated by calculating the level of FcγR-binding (RU) per unit amount of antibody using Biacore T200 Evaluation Software version 3.2.1.

Table 11 shows binding levels per unit amount of antibody, and Table 12 shows relative levels (%) to the binding level of the Fc control antibody (Sample No. 61) (the non-introduction of modifications is denoted as “−”). The antibodies with CH3 modifications that promote heavy chain homodimer formation generally showed comparable or slightly higher binding activity to each human FcγR as compared to the control. However, Sample No. 117 showed a binding level of half that of the control or lower for some FcγRs. Therefore, if Fc functions comparable to the control antibody are desired for the purpose of use, the CH3 modifications other than those of Sample No. 117 should be used.

TABLE 11
Response values of CH3-modified antibodies to human FcγRs per RU of captured antibody

| Sample | Modifications that promote formation | Level of binding to FcγR/amount of captured antibody | | |
|---|---|---|---|---|
| No. | of heavy chain homodimers | hFcgRIa | hFcgRIIa_167H | hFcgRIIa_167R |
| 61 | — | 0.2221 | 0.0637 | 0.0512 |
| 80 | T394W/F405A | 0.2143 | 0.0745 | 0.0641 |
| 87 | T366W/L368A/Y407V | 0.2087 | 0.0784 | 0.0673 |
| 92 | Q347R/K360E/D399V/F405T/K409W | 0.2235 | 0.0815 | 0.0690 |
| 95 | T394F/F405A | 0.2160 | 0.0815 | 0.0711 |
| 97 | E356K/D399K/K409E/K439E | 0.1818 | 0.0664 | 0.0537 |
| 100 | Q347E/S354Y/T366Y/Y407T | 0.2242 | 0.0633 | 0.0583 |
| 102 | K392L/T394W/F405A/Y407V | 0.2195 | 0.0857 | 0.0751 |
| 109 | E356K/K392D/D399K/K439E | 0.2011 | 0.0831 | 0.0676 |
| 111 | K392D/D399K/K409D | 0.2237 | 0.0839 | 0.0698 |
| 112 | Y349C/E357C/K392D/D399K | 0.2392 | 0.0889 | 0.0747 |
| 116 | E345R/Q347R/K360D/T366V/D399M/Y407A/ | 0.1823 | 0.0475 | 0.0424 |
| | K409V | | | |
| 117 | E356K/K392D/D399K/K409D | 0.2067 | 0.0343 | 0.0245 |
| 119 | E345R/K360E/D399M/Y407A/K409V | 0.1732 | 0.0561 | 0.0480 |
| 121 | T350V/L351Y/T366L/K392L/T394W/F405A/ | 0.1957 | 0.0927 | 0.0839 |
| | Y407V | | | |

| Sample | Level of binding to FcγR/amount of captured antibody | | | | |
|---|---|---|---|---|---|
| No. | hFcgRIIb | hFcgRIIIa_176F | hFcgRIIIa_176V | hFcgRIIIb_NA1 | hFcgRIIIb_NA2 |
| 61 | 0.0124 | 0.0281 | 0.0879 | 0.0058 | 0.0097 |
| 80 | 0.0191 | 0.0345 | 0.0977 | 0.0078 | 0.0123 |
| 87 | 0.0201 | 0.0427 | 0.1104 | 0.0097 | 0.0153 |
| 92 | 0.0204 | 0.0379 | 0.1036 | 0.0097 | 0.0148 |
| 95 | 0.0226 | 0.0382 | 0.1053 | 0.0095 | 0.0144 |
| 97 | 0.0135 | 0.0276 | 0.0874 | 0.0065 | 0.0100 |
| 100 | 0.0177 | 0.0406 | 0.1104 | 0.0078 | 0.0125 |
| 102 | 0.0255 | 0.0422 | 0.1084 | 0.0108 | 0.0163 |
| 109 | 0.0191 | 0.0368 | 0.0995 | 0.0094 | 0.0148 |
| 111 | 0.0197 | 0.0405 | 0.1042 | 0.0111 | 0.0167 |
| 112 | 0.0227 | 0.0405 | 0.1044 | 0.0113 | 0.0176 |
| 116 | 0.0111 | 0.0299 | 0.0804 | 0.0054 | 0.0087 |
| 117 | 0.0049 | 0.0171 | 0.0545 | 0.0019 | 0.0043 |
| 119 | 0.0120 | 0.0311 | 0.0876 | 0.0063 | 0.0103 |
| 121 | 0.0316 | 0.0518 | 0.1183 | 0.0143 | 0.0214 |

TABLE 12
Relative binding ratios (%) of CH3-modified antibodies for human FcγRs

| Sample | Modifications that promote formation | Ratio of (level of binding to FcγR/amount of captured antibody) to WT-IgG1 (%) | | |
|---|---|---|---|---|
| No. | of heavy chain homodimers | hFcgRIa | hFcgRIIa_167H | hFcgRIIa_167R |
| 61 | — | 100 | 100 | 100 |
| 80 | T394W/F405A | 96 | 117 | 125 |
| 87 | T366W/L368A/Y407V | 94 | 123 | 131 |
| 92 | Q347R/K360E/D399V/F405T/K409W | 101 | 128 | 135 |
| 95 | T394F/F405A | 97 | 128 | 139 |
| 97 | E356K/D399K/K409E/K439E | 82 | 104 | 105 |
| 100 | Q347E/S354Y/T366Y/Y407T | 101 | 99 | 114 |
| 102 | K392L/T394W/F405A/Y407V | 99 | 135 | 147 |
| 109 | E356K/K392D/D399K/K439E | 91 | 131 | 132 |
| 111 | K392D/D399K/K409D | 101 | 132 | 136 |
| 112 | Y349C/E357C/K392D/D399K | 108 | 140 | 146 |
| 116 | E345R/Q347R/K360D/T366V/D399M/Y407A/ | 82 | 75 | 83 |
| | K409V | | | |
| 117 | E356K/K392D/D399K/K409D | 93 | 54 | 48 |
| 119 | E345R/K360E/D399M/Y407A/K409V | 78 | 88 | 94 |
| 121 | T350V/L351Y/T366L/K392L/T394W/F405A/ | 88 | 146 | 164 |
| | Y407V | | | |

| Sample | Ratio of (level of binding to FcγR/amount of captured antibody) to WT-IgG1 (%) | | | | |
|---|---|---|---|---|---|
| No. | hFcgRIIb | hFcgRIIIa_176F | hFcgRIIIa_176V | hFcgRIIIb_NA1 | hFcgRIIIb_NA2 |
| 61 | 100 | 100 | 100 | 100 | 100 |
| 80 | 155 | 123 | 111 | 135 | 127 |
| 87 | 163 | 152 | 126 | 168 | 159 |
| 92 | 165 | 135 | 118 | 168 | 153 |
| 95 | 183 | 136 | 120 | 164 | 149 |
| 97 | 109 | 98 | 99 | 111 | 104 |
| 100 | 143 | 144 | 126 | 134 | 129 |
| 102 | 206 | 150 | 123 | 186 | 169 |
| 109 | 155 | 131 | 113 | 162 | 154 |
| 111 | 160 | 144 | 119 | 191 | 172 |
| 112 | 184 | 144 | 119 | 196 | 183 |
| 116 | 90 | 107 | 92 | 93 | 90 |
| 117 | 40 | 61 | 62 | 33 | 44 |
| 119 | 97 | 110 | 100 | 108 | 107 |
| 121 | 256 | 184 | 135 | 247 | 221 |

### [Example 9] Evaluation of ECM Binding of Antibodies with Modifications Introduced into CH3 Region

Next, the effect of heavy-chain homomeric association-promoting modifications on the non-specific binding of antibody was examined.

A system of evaluating binding to extracellular matrix (ECM) is known as an in vitro evaluation system for non-specific binding. This assay system was used for evaluation (US Patent 2014/0080153). Measurement results are shown in FIG. 4. The presence and absence of the modifications in the CH3 interface resulted in similar levels of ECM binding.

### [Example 10] Evaluation of Heavy-Chain Homodimer Formation Ability Between Different IgG Subclasses

In Examples 1-9, antibodies of IgG1 subclass were used for evaluation. However, other subclasses (IgG2 and IgG4) are also important for therapeutic antibodies. Moreover, for prevention of association with endogenous antibodies in living organisms, it is important that no heavy chain heteromers are formed between different subclasses. Here, some of the modifications that exhibited strong heavy-chain homodimer formation ability in IgG1 were applied to different IgG subclasses, and their heavy-chain homodimer formation ability was examined by SEC analysis. The correspondences between the template subclasses used for antibody expression, amino acid modifications, SEQ ID NOs, and area ratios are shown in Table 13 (the non-introduction of modifications is denoted as “−”). Antibody expression and analysis were performed according to the methods described in Example 1. However, pairs containing IgG3 were purified using MonoSpin ProG (GL science) as their affinity purification with Protein A is difficult.

The results of Table 13 showed that all subclass pairs of IgG1, IgG2, and IgG4 have heavy-chain homodimer formation ability.

In addition, the analysis of WT-IgG1, IgG2, and IgG4 paired with WT-IgG3 revealed that WT-IgG1, IgG2, and IgG4 intrinsically less readily form a pair with IgG3. This suggested that these amino acid-modified antibodies which do not form heavy-chain heterodimers between IgG1, IgG2, and IgG4 even less readily form heavy-chain heteromultimers with WT-IgG3.

TABLE 13
Evaluation of heavy-chain homodimer forming ability of pairs of different IgG subclasses

| | Full- | | | | | | | Homo | | |
|---|---|---|---|---|---|---|---|---|---|---|
| | length | | | | | | | full- | | |
| | heavy | | | Fc | | | Light | length | | |
| | chain | | Modifications that | fragment | | Modifications that | chain | heavy | Hetero- | Homo Fc |
| Sample | SEQ | IgG | promote formation of | SEQ ID | IgG | promote formation of | SEQ | chain | multimer | fragment |
| No. | ID NO | subclass | heavy chain homodimers | NO | subclass | heavy chain homodimers | ID NO | (%) | (%) | (%) |
| 169 | 1 | IgG1 | — | 62 | IgG1 | — | 61 | 28.7 | 52.1 | 19.2 |
| 170 | 86 | IgG2 | — | 62 | IgG1 | — | 61 | 21.6 | 55 | 23.5 |
| 171 | 87 | IgG2 | T394W/F405A | 62 | IgG1 | — | 61 | 58.9 | 0 | 41.1 |
| 172 | 88 | IgG2 | T366W/L368A/Y407V | 62 | IgG1 | — | 61 | 48.2 | 0 | 51.8 |
| 173 | 89 | IgG2 | Q347R/K360E/D399V/F405T/K409W | 62 | IgG1 | — | 61 | 55.9 | 0 | 44.1 |
| 174 | 90 | IgG2 | K392L/T394W/F405A/Y407V | 62 | IgG1 | — | 61 | 48.8 | 0 | 51.2 |
| 175 | 91 | IgG2 | K392D/D399K/K409D | 62 | IgGI | — | 61 | 52.7 | 0 | 47.3 |
| 176 | 86 | IgG2 | — | 102 | IgG4 | — | 61 | 18.2 | 60.4 | 21.4 |
| 177 | 87 | IgG2 | T394W/F405A | 102 | IgG4 | — | 61 | 66.7 | 0 | 33.3 |
| 178 | 88 | IgG2 | T366W/L368A/Y407V | 102 | IgG4 | — | 61 | 66.6 | 0 | 33.4 |
| 179 | 89 | IgG2 | Q347R/K360E/D399V/F405T/K409W | 102 | IgG4 | — | 61 | 66.8 | 0 | 33.2 |
| 180 | 90 | IgG2 | K392L/T394W/F405A/Y407V | 102 | IgG4 | — | 61 | 66.3 | 0 | 33.7 |
| 181 | 91 | IgG2 | K392D/D399K/K409D | 102 | IgG4 | — | 61 | 60.5 | 0 | 39.5 |
| 182 | 92 | IgG4 | — | 62 | IgG1 | — | 61 | 46.4 | 47.7 | 5.8 |
| 183 | 93 | IgG4 | T394W/F405A | 62 | IgG1 | — | 61 | 56.3 | 0 | 43.7 |
| 184 | 94 | IgG4 | T366W/L368A/Y407V | 62 | IgG1 | — | 61 | 87.3 | 0 | 12.7 |
| 185 | 95 | IgG4 | Q347R/K360E/D399V/F405T/R409W | 62 | IgG1 | — | 61 | 58.6 | 0 | 41.4 |
| 186 | 96 | IgG4 | K392L/T394W/F405A/Y407V | 62 | IgG1 | — | 61 | 78.7 | 0.2 | 21 |
| 187 | 97 | IgG4 | K392D/D399K/R409D | 62 | IgG1 | — | 61 | 56.5 | 0 | 43.5 |
| 188 | 92 | IgG4 | — | 102 | IgG4 | — | 61 | 19.4 | 54.3 | 26. |
| 189 | 93 | IgG4 | T394W/F405A | 102 | IgG4 | — | 61 | 60.3 | 0 | 39.7 |
| 190 | 94 | IgG4 | T366W/L368A/Y407V | 102 | IgG4 | — | 61 | 65.3 | 0 | 34.7 |
| 191 | 95 | IgG4 | Q347R/K360E/D399V/F405T/R409W | 102 | IgG4 | — | 61 | 66.8 | 0 | 33.2 |
| 192 | 96 | IgG4 | K392L/T394W/F405A/Y407V | 102 | IgG4 | — | 61 | 66.2 | 0 | 33.8 |
| 193 | 97 | IgG4 | K392D/D399K/R409D | 102 | IgG4 | — | 61 | 64.5 | 0 | 35.5 |
| 194 | 1 | IgG1 | — | 102 | IgG4 | — | 61 | 33.8 | 55.3 | 10.9 |
| 195 | 19 | IgG1 | T394W/F405A | 102 | IgG4 | — | 61 | 75.3 | 0 | 24.7 |
| 196 | 26 | IgG1 | T366W/L368A/Y407V | 102 | IgG4 | — | 61 | 73.8 | 0 | 26.2 |
| 197 | 31 | IgG1 | Q347R/K360E/D399V/F405T/K409W | 102 | IgG4 | — | 61 | 72.1 | 0 | 27.9 |
| 198 | 36 | IgG1 | E356K/D399K/K409E/K439E | 102 | IgG4 | — | 61 | 73.3 | 0 | 26.7 |
| 199 | 39 | IgG1 | Q347E/S354Y/T366Y/Y407T | 102 | IgG4 | — | 61 | 70.4 | 0 | 29.6 |
| 200 | 41 | IgG1 | K392L/T394W/F405A/Y407V | 102 | IgG4 | — | 61 | 72.7 | 0 | 27.3 |
| 201 | 48 | IgG1 | E356K/K392D/D399K/K439E | 102 | IgG4 | — | 61 | 69.6 | 0 | 30.4 |
| 202 | 50 | IgG1 | K392D/D399K/K409D | 102 | IgG4 | — | 61 | 76.8 | 0 | 23.2 |
| 203 | 55 | IgG1 | E345R/Q347R/K360D/T366V/D399M/Y407A/ | 102 | IgG4 | — | 61 | 70.7 | 0 | 29.3 |
| | | | K409V | | | | | | | |
| 204 | 56 | IgG1 | E356K/K392D/D399K/K409D | 102 | IgG4 | — | 61 | 70.7 | 0 | 29.3 |
| 205 | 58 | IgG1 | E345R/K360E/D399M/Y407A/K409V | 102 | IgG4 | — | 61 | 70.3 | 0 | 29.7 |
| 206 | 60 | IgG1 | T350V/L351Y/T366L/K392L/T394W/F405A/ | 102 | IgG4 | — | 61 | 71.9 | 0 | 28.1 |
| | | | Y407V | | | | | | | |
| 207 | 86 | IgG2 | — | 103 | IgG2 | — | 61 | 37.8 | 48.8 | 13.4 |
| 208 | 87 | IgG2 | T394W/F405A | 103 | IgG2 | — | 61 | 80.8 | 0 | 19.2 |
| 209 | 88 | IgG2 | T366W/L368A/Y407V | 103 | IgG2 | — | 61 | 80 | 0 | 20 |
| 210 | 89 | IgG2 | Q347R/K360E/D399V/F405T/K409W | 103 | IgG2 | — | 61 | 80.4 | 0 | 19.6 |
| 211 | 90 | IgG2 | K392L/T394W/F405A/Y407V | 103 | IgG2 | — | 61 | 78.5 | 0 | 21.5 |
| 212 | 91 | IgG2 | K392D/D399K/K409D | 103 | IgG2 | — | 61 | 79.6 | 0 | 20.4 |
| 213 | 92 | IgG4 | — | 103 | IgG2 | — | 61 | 31.8 | 56.1 | 12.1 |
| 214 | 93 | IgG4 | T394W/F405A | 103 | IgG2 | — | 61 | 82 | 0 | 18 |
| 215 | 94 | IgG4 | T366W/L368A/Y407V | 103 | IgG2 | — | 61 | 83 | 0 | 17 |
| 216 | 95 | IgG4 | Q347R/K360E/D399V/F405T/R409W | 103 | IgG2 | — | 61 | 83.5 | 0 | 16.5 |
| 217 | 96 | IgG4 | K392L/T394W/F405A/Y407V | 103 | IgG2 | — | 61 | 81.5 | 0 | 18.5 |
| 218 | 97 | IgG4 | K392D/D399K/R409D | 103 | IgG2 | — | 61 | 82.5 | 0 | 17.5 |
| 219 | 1 | IgG1 | | 103 | IgG2 | — | 61 | 38.7 | 50 | 11.3 |
| 220 | 19 | IgG1 | T394W/F405A | 103 | IgG2 | — | 61 | 84.3 | 0 | 15.7 |
| 221 | 26 | IgG1 | T366W/L368A/Y407V | 103 | IgG2 | — | 61 | 85.3 | 0 | 14.7 |
| 222 | 31 | IgG1 | Q347R/K360E/D399V/F405T/K409W | 103 | IgG2 | — | 61 | 82.7 | 0 | 17.3 |
| 223 | 36 | IgG1 | E356K/D399K/K409E/K439E | 103 | IgG2 | — | 61 | 84.3 | 0 | 15.7 |
| 224 | 39 | IgG1 | Q347E/S354Y/T366Y/Y407T | 103 | IgG2 | — | 61 | 82.9 | 0 | 17.1 |
| 225 | 41 | IgG1 | K392L/T394W/F405A/Y407V | 103 | IgG2 | — | 61 | 83.8 | 0 | 16.2 |
| 226 | 48 | IgG1 | E356K/K392D/D399K/K439E | 103 | IgG2 | — | 61 | 85.1 | 0 | 14.9 |
| 227 | 50 | IgG1 | K392D/D399K/K409D | 103 | IgG2 | — | 61 | 81.4 | 0 | 18.6 |
| 228 | 55 | IgG1 | E345R/Q347R/K360D/T366V/D399M/Y407A/ | 103 | IgG2 | — | 61 | 83.5 | 0 | 16.5 |
| | | | K409V | | | | | | | |
| 229 | 56 | IgG1 | E356K/K392D/D399K/K409D | 103 | IgG2 | — | 61 | 81.4 | 0 | 18.6 |
| 230 | 58 | IgG1 | E345R/K360E/D399M/Y407A/K409V | 103 | IgG2 | — | 61 | 84.3 | 0 | 15.7 |
| 231 | 60 | IgG1 | T350V/L351Y/T366L/K392L/T394W/F405A/ | 103 | IgG2 | — | 61 | 85.8 | 0 | 14.2 |
| | | | Y407V | | | | | | | |
| 232 | 98 | IgG2 | E356K/D399K/K409E | 103 | IgG2 | — | 61 | 51.1 | 8.2 | 40.7 |
| 233 | 99 | IgG2 | Q347E/S354Y/T366Y/Y407T | 103 | IgG2 | — | 61 | 76.3 | 0 | 23.7 |
| 234 | 100 | IgG2 | E345R/Q347R/K360D/T366V/D399M/Y407A/ | 103 | IgG2 | — | 61 | 76.1 | 0 | 23.9 |
| | | | K409V | | | | | | | |
| 235 | 101 | IgG3 | — | 62 | IgG1 | — | 61 | 49.3 | 20 | 30.6 |
| 236 | 101 | IgG3 | — | 103 | IgG2 | — | 61 | 37.2 | 37.4 | 25.4 |
| 237 | 101 | IgG3 | — | 102 | IgG4 | — | 61 | 62.2 | 10.4 | 27.3 |
| 238 | 104 | IgG2 | E356K/K392D/D399K/K439E | 62 | IgG1 | — | 61 | 50.9 | 0 | 49.1 |
| 239 | 98 | IgG2 | E356K/D399K/K409E | 62 | IgG1 | — | 61 | 20. | 5.4 | 73.9 |
| 240 | 99 | IgG2 | Q347E/S354Y/T366Y/Y407T | 62 | IgG1 | — | 61 | 46.9 | 0 | 53.1 |
| 241 | 100 | IgG2 | E345R/Q347R/K360D/T366V/D399M/Y407A/ | 62 | IgG1 | — | 61 | 49.3 | 0 | 50.7 |
| | | | K409V | | | | | | | |
| 242 | 105 | IgG2 | E356K/K392D/D399K/K409D | 62 | IgG1 | — | 61 | 44.1 | 0 | 55.9 |
| 243 | 106 | IgG2 | E345R/K360E/D399M/Y407A/K409V | 62 | IgG1 | — | 61 | 49.8 | 0 | 50.2 |
| 244 | 107 | IgG2 | T350V/L351Y/T366L/K392L/T394W/F405A/ | 62 | IgG1 | — | 61 | 47.6 | 0 | 52.4 |
| | | | Y407V | | | | | | | |
| 245 | 108 | IgG4 | E356K/K392D/D399K/K439E | 62 | IgG1 | — | 61 | 48.6 | 0 | 51.4 |
| 246 | 109 | IgG4 | E356K/D399K/R409E/K439E | 62 | IgG1 | — | 61 | 56.4 | 0 | 43.6 |
| 247 | 110 | IgG4 | Q347E/S354Y/T366Y/Y407T | 62 | IgG1 | — | 61 | 39 | 10.6 | 50.5 |
| 248 | 111 | IgG4 | E345R/Q347R/K360D/T366V/D399M/Y407A/ | 62 | IgG1 | — | 61 | 54 | 0 | 46 |
| | | | K409V | | | | | | | |
| 249 | 112 | IgG4 | E356K/K392D/D399K/R409D | 62 | IgG1 | — | 61 | 55.8 | 0 | 44.2 |
| 250 | 113 | IgG4 | E345R/K360E/D399M/Y407A/R409V | 62 | IgG1 | — | 61 | 60.2 | 0 | 39.8 |
| 251 | 114 | IgG4 | T350V/L351Y/T366L/K392L/T394W/F405A/ | 62 | IgG1 | — | 61 | 55.1 | 0 | 44.9 |
| | | | Y407V | | | | | | | |
| 252 | 104 | IgG2 | E356K/K392D/D399K/K439E | 103 | IgG2 | — | 61 | 82.7 | 0 | 17.3 |
| 253 | 98 | IgG2 | E356K/D399K/K409E | 103 | IgG2 | — | 61 | 63.7 | 8.7 | 27.6 |
| 254 | 99 | IgG2 | Q347E/S354Y/T366Y/Y407T | 103 | IgG2 | — | 61 | 81.5 | 0 | 18.5 |
| 255 | 100 | IgG2 | E345R/Q347R/K360D/T366V/D399M/Y407A/ | 103 | IgG2 | — | 61 | 80.8 | 0 | 19.2 |
| | | | K409V | | | | | | | |
| 256 | 105 | IgG2 | E356K/K392D/D399K/K409D | 103 | IgG2 | — | 61 | 78.2 | 0 | 21.8 |
| 257 | 106 | IgG2 | E345R/K360E/D399M/Y407A/K409V | 103 | IgG2 | — | 61 | 83.2 | 0 | 16.8 |
| 258 | 108 | IgG4 | E356K/K392D/D399K/K439E | 103 | IgG2 | — | 61 | 81.9 | 0 | 18.1 |
| 259 | 109 | IgG4 | E356K/D399K/R409E/K439E | 103 | IgG2 | — | 61 | 86.2 | 0 | 13.8 |
| 260 | 110 | IgG4 | Q347E/S354Y/T366Y/Y407T | 103 | IgG2 | — | 61 | 73.4 | 5.3 | 21.3 |
| 261 | 111 | IgG4 | E345R/Q347R/K360D/T366V/D399M/Y407A/ | 103 | IgG2 | — | 61 | 82 | 0 | 18 |
| | | | R409V | | | | | | | |
| 262 | 112 | IgG4 | E356K/K392D/D399K/R409D | 103 | IgG2 | — | 61 | 82.8 | 0 | 17.2 |
| 263 | 113 | IgG4 | E345R/K360E/D399M/Y407A/R409V | 103 | IgG2 | — | 61 | 84.5 | 0 | 15.5 |
| 264 | 114 | IgG4 | T350V/L351Y/T366L/K392L/T394W/F405A/ | 103 | IgG2 | — | 61 | 85.6 | 0 | 14.4 |
| | | | Y407V | | | | | | | |
| 265 | 104 | IgG2 | E356K/K392D/D399K/K439E | 102 | IgG4 | — | 61 | 61.3 | 0 | 38.7 |
| 266 | 98 | IgG2 | E356K/D399K/K409E | 102 | IgG4 | — | 61 | 24.9 | 0 | 75.1 |
| 267 | 99 | IgG2 | Q347E/S354Y/T366Y/Y407T | 102 | IgG4 | — | 61 | 56.5 | 0 | 43.5 |
| 268 | 100 | IgG2 | E345R/Q347R/K360D/T366V/D399M/Y407A/ | 102 | IgG4 | — | 61 | 57.7 | 0 | 42.3 |
| | | | K409V | | | | | | | |
| 269 | 105 | IgG2 | E356K/K392D/D399K/K409D | 102 | IgG4 | — | 61 | 50.8 | 0 | 49.2 |
| 270 | 106 | IgG2 | E345R/K360E/D399M/Y407A/K409V | 102 | IgG4 | — | 61 | 62.8 | 0 | 37.2 |
| 271 | 107 | IgG2 | T350V/L351Y/T366L/K392L/T394W/F405A/ | 102 | IgG4 | — | 61 | 59.1 | 0 | 40.9 |
| | | | Y407V | | | | | | | |
| 272 | 108 | IgG4 | E356K/K392D/D399K/K439E | 102 | IgG4 | — | 61 | 58 | 0 | 42 |
| 273 | 109 | IgG4 | E356K/D399K/R409E/K439E | 102 | IgG4 | — | 61 | 64.9 | 0 | 35.1 |
| 274 | 110 | IgG4 | Q347E/S354Y/T366Y/Y407T | 102 | IgG4 | — | 61 | 51.3 | 4.6 | 44.1 |
| 275 | 111 | IgG4 | E345R/Q347R/K360D/T366V/D399M/Y407A/ | 102 | IgG4 | — | 61 | 60.8 | 0 | 39.2 |
| | | | R409V | | | | | | | |
| 276 | 112 | IgG4 | E356K/K392D/D399K/R409D | 102 | IgG4 | — | 61 | 62.6 | 0 | 37.4 |
| 277 | 113 | IgG4 | E345R/K360E/D399M/Y407A/R409V | 102 | IgG4 | — | 61 | 67 | 0 | 33 |
| 278 | 114 | IgG4 | T350V/L351Y/T366L/K392L/T394W/F405A/ | 102 | IgG4 | — | 61 | 64 | 0 | 36 |
| | | | Y407V | | | | | | | |
| 279 | 107 | IgG2 | T350V/L351Y/T366L/K392L/T394W/F405A/ | 103 | IgG2 | — | 61 | 85.5 | 0 | 14.5 |
| | | | Y407V | | | | | | | |

### [Example 11] Evaluation of Heavy Chain Homodimer-Forming Ability in Antibodies Containing Different IgG Formats and Fc Modifications

The evaluation of Examples 1-10 was performed using the basic IgG format and the Fc of wild-type IgG containing no other modification than CH3 interface control. However, to prepare and express more effective therapeutic antibodies, applicability to various antibody formats, such as formats in which a VHH or scFv is linked with an Fc, and antibodies into which amino acid modifications for altering Fc functions are introduced, is ideal. Here, some of the modifications that exhibited strong heavy-chain homodimer formation ability in IgG were applied to various antibody formats and Fc-modified antibodies, and their heavy chain homodimer formation ability was examined by SEC analysis. The correspondences between the heavy chain backbone or amino acid modifications (and their functions) contained in the template antibodies used for antibody expression, the amino acid modifications for promoting heavy chain homodimer formation, the SEQ ID NOs, and the area ratios are shown in Table 14 (the non-introduction of modifications is denoted as “−”). Antibody expression and analysis were performed according to the methods described in Example 1. However, for antibodies containing the E345R/E430G/S440Y modification, which promotes hexamer formation of IgG, a multimeric component was detected in SEC analysis. In addition, for pairs containing the format of scFv.LH-Fc or scFv.HL-Fc, complete separation of each homomer and heteromeric multimer was difficult. Thus, for these samples, the effect of suppressing heteromultimer formation was qualitatively evaluated focusing on the peak of the heteromultimeric component (FIGS. 5-1 to 5-14).

Based on the results of Table 14 and FIGS. 5-1 to 5-14, it was revealed that the heavy-chain homodimer formation ability (or the effect of suppressing heteromultimer formation) is maintained even when the binding region of the antibody is VHH or scFv, and even when the Fc region is deglycosylated or contains modifications for enhancing FcRn binding, suppressing FcγR binding, or promoting hexamer formation.

Furthermore, since the heavy-chain homodimer formation ability (or the effect of suppressing heterodimer formation) is maintained in all formats, it can expected to be also maintained even when Fc is linked to a polypeptide other than Fab, VHH, and scFv, and widely maintained even in the presence of amino acid modifications that do not affect the Fc interface.

TABLE 14
Evaluation of heavy-chain homodimer formation ability in antibodies including VHH-Fc, scFv-Fc, and modifications

| | | | Full- | | Fc | Homo | | |
|---|---|---|---|---|---|---|---|---|
| | | | length | | fragment | full- | | |
| | | | heavy | Light | or scFv, | length | | |
| | | | chain | chain | VHH-Fc | heavy | Hetero- | Homo Fc |
| Sample | Heavy chain backbone or amino acid | Modifications that promote formation | SEQ | SEQ | SEQ ID | chain | multimer | fragment |
| No. | modifications (functions) contained | of heavy chain homodimers | ID NO | ID NO | NO | (%) | (%) | (%) |
| 280 | N297A (deglycosylation) | — | 115 | 61 | 62 | 26.0 | 50.2 | 23.9 |
| 281 | N297A (deglycosylation) | T394W/F405A | 116 | 61 | 62 | 63.3 | 0.0 | 36.7 |
| 282 | N297A (deglycosylation) | T366W/L368A/Y407V | 117 | 61 | 62 | 57.7 | 2.3 | 40.0 |
| 283 | N297A (deglycosylation) | Q347R/K360E/D399V/F405T/K409W | 118 | 61 | 62 | 61.4 | 0.0 | 38.6 |
| 284 | N297A (deglycosylation) | E356K/D399K/K409E/K439E | 119 | 61 | 62 | 62.6 | 0.0 | 37.4 |
| 285 | N297A (deglycosylation) | E356K/K392D/D399K/K439E | 120 | 61 | 62 | 59.8 | 2.6 | 37.6 |
| 286 | N297A (deglycosylation) | K392D/D399K/K409D | 121 | 61 | 62 | 57.1 | 1.5 | 41.3 |
| 287 | N297A (deglycosylation) | E345R/K360E/D399M/Y407A/K409V | 122 | 61 | 62 | 66.3 | 0.0 | 33.7 |
| 288 | N297A (deglycosylation) | T350V/L351Y/T366L/K392L/T394W/F405A/Y407V | 123 | 61 | 62 | 60.2 | 0.0 | 39.8 |
| 289 | M428L/N434A/Y436T/Q438R/S440E | — | 124 | 61 | 62 | 18.6 | 54.5 | 26.8 |
| | (enhance FcRn binding) | | | | | | | |
| 290 | M428L/N434A/Y436T/Q438R/S440E | T394W/F405A | 125 | 61 | 62 | 43.4 | 1.6 | 55.1 |
| | (enhance FcRn binding) | | | | | | | |
| 291 | M428L/N434A/Y436T/Q438R/S440E | T366W/L368A/Y407V | 126 | 61 | 62 | 51.9 | 2.4 | 45.7 |
| | (enhance FcRn binding) | | | | | | | |
| 292 | M428L/N434A/Y436T/Q438R/S440E | Q347R/K360E/D399V/F405T/K409W | 127 | 61 | 62 | 49.9 | 1.5 | 48.6 |
| | (enhance FcRn binding) | | | | | | | |
| 293 | M428L/N434A/Y436T/Q438R/S440E | E356K/D399K/K409E/K439E | 128 | 61 | 62 | 51.7 | 1.5 | 46.8 |
| | (enhance FcRn binding) | | | | | | | |
| 294 | M428L/N434A/Y436T/Q438R/S440E | E356K/K392D/D399K/K439E | 129 | 61 | 62 | 58.8 | 2.2 | 39.1 |
| | (enhance FcRn binding) | | | | | | | |
| 295 | M428L/N434A/Y436T/Q438R/S440E | K392D/D399K/K409D | 130 | 61 | 62 | 43.2 | 1.6 | 55.2 |
| | (enhance FcRn binding) | | | | | | | |
| 296 | M428L/N434A/Y436T/Q438R/S440E | E345R/K360E/D399M/Y407A/K409V | 131 | 61 | 62 | 45.4 | 1.7 | 52.9 |
| | (enhance FcRn binding) | | | | | | | |
| 297 | M428L/N434A/Y436T/Q438R/S440E | T350V/L351Y/T366L/K392L/T394W/F405A/Y407V | 132 | 61 | 62 | 52.0 | 0.0 | 48.0 |
| | (enhance FcRn binding) | | | | | | | |
| 298 | VHH-Fc | — | 1 | 61 | 579 | 52.2 | 41.8 | 5.9 |
| 299 | VHH-Fc | T394W/F405A | 1 | 61 | 580 | 68.9 | 0.0 | 31.1 |
| 300 | VHH-Fc | T366W/L368A/Y407V | 1 | 61 | 581 | 69.5 | 0.0 | 30.5 |
| 301 | VHH-Fc | Q347R/K360E/D399V/F405T/K409W | 1 | 61 | 582 | 72.8 | 0.0 | 27.2 |
| 302 | VHH-Fc | T394F/F405A | 1 | 61 | 583 | 73.6 | 0.0 | 26.4 |
| 303 | VHH-Fc | E356K/D399K/K409E/K439E | 1 | 61 | 584 | 73.4 | 0.0 | 26.6 |
| 304 | VHH-Fc | Q347E/S354Y/T366Y/Y407T | 1 | 61 | 585 | 68.8 | 0.0 | 31.2 |
| 305 | VHH-Fc | K392L/T394W/F405A/Y407V | 1 | 61 | 586 | 66.1 | 0.0 | 33.9 |
| 306 | VHH-Fc | E356K/K392D/D399K/K439E | 1 | 61 | 587 | 68.5 | 0.0 | 31.5 |
| 307 | VHH-Fc | K392D/D399K/K409D | 1 | 61 | 588 | 75.7 | 0.0 | 24.3 |
| 308 | VHH-Fc | Y349C/E357C/K392D/D399K | 1 | 61 | 589 | 91.4 | 0.0 | 8.6 |
| 309 | VHH-Fc | E345R/Q347R/K360D/T366V/D399M/Y407A/K409V | 1 | 61 | 590 | 67.7 | 0.0 | 32.3 |
| 310 | VHH-Fc | E356K/K392D/D399K/K409D | 1 | 61 | 591 | 71.1 | 0.0 | 28.9 |
| 311 | VHH-Fc | E345R/K360E/D399M/Y407A/K409V | 1 | 61 | 592 | 67.3 | 0.0 | 32.7 |
| 312 | VHH-Fc | T350V/L351Y/T366L/K392L/T394W/F405A/Y407V | 1 | 61 | 593 | 68.2 | 0.0 | 31.8 |
| 313 | L235R/G236R (suppress FcγR binding) | — | 133 | 61 | 594 | 27.9 | 52.9 | 19.2 |
| 314 | L235R/G236R (suppress FcγR binding) | T394W/F405A | 134 | 61 | 594 | 64.1 | 0.0 | 35.9 |
| 315 | L235R/G236R (suppress FcγR binding) | T366W/L368A/Y407V | 135 | 61 | 594 | 58.1 | 3.4 | 38.5 |
| 316 | L235R/G236R (suppress FcγR binding) | Q347R/K360E/D399V/F405T/K409W | 136 | 61 | 594 | 60.2 | 0.0 | 39.8 |
| 317 | L235R/G236R (suppress FcγR binding) | E356K/K392D/D399K/K439E | 137 | 61 | 594 | 62.2 | 0.0 | 37.8 |
| 318 | L234A/L235A (suppress FcγR binding) | — | 138 | 61 | 595 | 36.0 | 50.3 | 13.7 |
| 319 | L234A/L235A (suppress FcγR binding) | T394W/F405A | 139 | 61 | 595 | 66.9 | 0.0 | 33.1 |
| 320 | L234A/L235A (suppress FcγR binding) | T366W/L368A/Y407V | 140 | 61 | 595 | 67.5 | 0.0 | 32.5 |
| 321 | L234A/L235A (suppress FcγR binding) | Q347R/K360E/D399V/F405T/K409W | 141 | 61 | 595 | 65.3 | 0.0 | 34.7 |
| 322 | L234A/L235A (suppress FcγR binding) | E356K/K392D/D399K/K439E | 142 | 61 | 595 | 67.9 | 0.0 | 32.1 |
| 323 | M252Y/S254T/T256E (enhance FcRn | — | 143 | 61 | 596 | 16.3 | 50.1 | 33.7 |
| | binding) | | | | | | | |
| 324 | M252Y/S254T/T256E (enhance FcRn | T394W/F405A | 144 | 61 | 596 | 69.8 | 0.0 | 30.2 |
| | binding) | | | | | | | |
| 325 | M252Y/S254T/T256E (enhance FcRn | T366W/L368A/Y407V | 145 | 61 | 596 | 62.3 | 0.0 | 37.7 |
| | binding) | | | | | | | |
| 326 | M252Y/S254T/T256E (enhance FcRn | Q347R/K360E/D399V/F405T/K409W | 146 | 61 | 596 | 63.3 | 0.0 | 36.7 |
| | binding) | | | | | | | |
| 327 | M252Y/S254T/T256E (enhance FcRn | E356K/K392D/D399K/K439E | 147 | 61 | 596 | 61.6 | 0.0 | 38.4 |
| | binding) | | | | | | | |
| 328 | M428L/N434S (enhance FcRn binding) | — | 148 | 61 | 597 | 32.6 | 51.7 | 15.8 |
| 329 | M428L/N434S (enhance FcRn binding) | T394W/F405A | 149 | 61 | 597 | 61.0 | 0.0 | 39.0 |
| 330 | M428L/N434S (enhance FcRn binding) | T366W/L368A/Y407V | 150 | 61 | 597 | 54.8 | 0.0 | 45.2 |
| 331 | M428L/N434S (enhance FcRn binding) | Q347R/K360E/D399V/F405T/K409W | 151 | 61 | 597 | 66.9 | 0.0 | 33.1 |
| 332 | M428L/N434S (enhance FcRn binding) | E356K/K392D/D399K/K439E | 152 | 61 | 597 | 67.9 | 0.0 | 32.1 |
| 333 | L234A/L235A/P329G (suppress FcγR | — | 153 | 61 | 598 | 22.6 | 51.4 | 26.0 |
| | binding) | | | | | | | |
| 334 | L234A/L235A/P329G (suppress FcγR | T394W/F405A | 154 | 61 | 598 | 62.8 | 0.0 | 37.2 |
| | binding) | | | | | | | |
| 335 | L234A/L235A/P329G (suppress FcγR | T366W/L368A/Y407V | 155 | 61 | 598 | 63.9 | 0.0 | 36.1 |
| | binding) | | | | | | | |
| 336 | L234A/L235A/P329G (suppress FcγR | Q347R/K360E/D399V/F405T/K409W | 156 | 61 | 598 | 61.5 | 0.0 | 38.5 |
| | binding) | | | | | | | |
| 337 | L234A/L235A/P329G (suppress FcγR | E356K/K392D/D399K/K439E | 157 | 61 | 598 | 57.4 | 0.0 | 42.6 |
| | binding) | | | | | | | |
| 338 | L234F/L235E/P331S (suppress FcγR | T394W/F405A | 158 | 61 | 599 | 61.9 | 0.0 | 38.1 |
| | binding) | | | | | | | |
| 339 | L234F/L235E/P331S (suppress FcγR | Q347R/K360E/D399V/F405T/K409W | 159 | 61 | 599 | 64.6 | 0.0 | 35.4 |
| | binding) | | | | | | | |
| 340 | L234F/L235E/P331S (suppress FcγR | E356K/K392D/D399K/K439E | 160 | 61 | 599 | 61.6 | 0.0 | 38.4 |
| | binding) | | | | | | | |
| 341 | E345R/E430G/S440Y (promote hexamer | — | 161 | 61 | 165 | Modified Fc control | | |
| | formation) | | | | | | | |
| 342 | E345R/E430G/S440Y (promote hexamer | T394W/F405A | 162 | 61 | 165 | Qualitative evaluation of decrease | | |
| | formation) | | | | | in heteromultimers | | |
| 343 | E345R/E430G/S440Y (promote hexamer | T366W/L368A/Y407V | 163 | 61 | 165 | Qualitative evaluation of decrease | | |
| | formation) | | | | | in heteromultimers | | |
| 344 | E345R/E430G/S440Y (promote hexamer | Q347R/K360E/D399V/F405T/K409W | 164 | 61 | 165 | Qualitative evaluation of decrease | | |
| | formation) | | | | | in heteromultimers | | |
| 345 | scFv.LH-Fc | — | 1 | 61 | 166 | scFv.LH-Fc control | | |
| 346 | scFv.LH-Fc | Q347R/K360E/D399V/F405T/K409W | 1 | 61 | 167 | Qualitative evaluation of decrease | | |
| | | | | | | in heteromultimers | | |
| 347 | scFv.LH-Fc | E356K/K392D/D399K/K439E | 1 | 61 | 168 | Qualitative evaluation of decrease | | |
| | | | | | | in heteromultimers | | |
| 348 | scFv.LH-Fc | T394W/F405A | 1 | 61 | 169 | Qualitative evaluation of decrease | | |
| | | | | | | in heteromultimers | | |
| 349 | scFv.LH-Fc | T366W/L368A/Y407V | 1 | 61 | 170 | Qualitative evaluation of decrease | | |
| | | | | | | in heteromultimers | | |
| 350 | scFv.HL-Fc | — | 1 | 61 | 171 | scFv.HL-Fc control | | |
| 351 | scFv.HL-Fc | Q347R/K360E/D399V/F405T/K409W | 1 | 61 | 172 | Qualitative evaluation of decrease | | |
| | | | | | | in heteromultimers | | |
| 352 | scFv.HL-Fc | E356K/K392D/D399K/K439E | 1 | 61 | 173 | Qualitative evaluation of decrease | | |
| | | | | | | in heteromultimers | | |
| 353 | scFv.HL-Fc | T394W/F405A | 1 | 61 | 174 | Qualitative evaluation of decrease | | |
| | | | | | | in heteromultimers | | |
| 354 | scFv.HL-Fc | T366W/L368A/Y407V | 1 | 61 | 175 | Qualitative evaluation of decrease | | |
| | | | | | | in heteromultimers | | |
| 355 | L234F/L235E/P331S (suppress FcγR | — | 1 | 61 | 599 | 25.5 | 51.4 | 23.1 |
| | binding) Fc fragment | | | | | | | |
| 356 | L234F/L235E/P331S (suppress FcγR | T394W/F405A | 1 | 61 | 600 | 62.1 | 0.0 | 37.9 |
| | binding) Fc fragment | | | | | | | |
| 357 | L234F/L235E/P331S (suppress FcγR | T366W/L368A/Y407V | 1 | 61 | 601 | 60.7 | 0.0 | 39.3 |
| | binding) Fc fragment | | | | | | | |
| 358 | L234F/L235E/P331S (suppress FcγR | Q347R/K360E/D399V/F405T/K409W | 1 | 61 | 602 | 61.2 | 0.0 | 38.8 |
| | binding) Fc fragment | | | | | | | |
| 359 | L234F/L235E/P331S (suppress FcγR | E356K/K392D/D399K/K439E | 1 | 61 | 603 | 62.3 | 0.0 | 37.7 |
| | binding) Fc fragment | | | | | | | |
| 360 | E345R/E430G/S440Y (promote hexamer | — | 1 | 61 | 165 | 30.1 | 61.1 | 8.8 |
| | formation) Fc fragment | | | | | | | |
| 361 | E345R/E430G/S440Y (promote hexamer | T394W/F405A | 1 | 61 | 604 | 86.1 | 0.0 | 13.9 |
| | formation) Fc fragment | | | | | | | |
| 362 | E345R/E430G/S440Y (promote hexamer | T366W/L368A/Y407V | 1 | 61 | 605 | 87.2 | 0.0 | 12.8 |
| | formation) Fc fragment | | | | | | | |
| 363 | E345R/E430G/S440Y (promote hexamer | Q347R/K360E/D399V/F405T/K409W | 1 | 61 | 606 | 92.0 | 0.0 | 8.0 |
| | formation) Fc fragment | | | | | | | |
| 364 | E345R/E430G/S440Y (promote hexamer | — | 161 | 61 | — | Reference sample | | |
| | formation) | | | | | | | |

| | | | | | | |
|---|---|---|---|---|---|---|
| 365 | E345R/E430G/S440Y (promote hexamer | T394W/F405A | 162 | 61 | — | Reference sample |
| | formation) | | | | | |
| 366 | E345R/E430G/S440Y (promote hexamer | T366W/L368A/Y407V | 163 | 61 | — | Reference sample |
| | formation) | | | | | |
| 367 | E345R/E430G/S440Y (promote hexamer | Q347R/K360E/D399V/F405T/K409W | 164 | 61 | — | Reference sample |
| | formation) | | | | | |
| 368 | E345R/E430G/S440Y (promote hexamer | — | — | — | 165 | Reference sample |
| | formation) | | | | | |
| 369 | WT-IgG1 | — | 1 | 61 | — | Reference sample |
| 370 | scFv.LH-Fc | — | — | — | 166 | Reference sample |
| 371 | scFv.LH-Fc | Q347R/K360E/D399V/F405T/K409W | — | — | 167 | Reference sample |
| 372 | scFv.LH-Fc | E356K/K392D/D399K/K439E | — | — | 168 | Reference sample |
| 373 | scFv.LH-Fc | T394W/F405A | — | — | 169 | Reference sample |
| 374 | scFv.LH-Fc | T366W/L368A/Y407V | — | — | 170 | Reference sample |
| 375 | scFv.HL-Fc | — | — | — | 171 | Reference sample |
| 376 | scFv.HL-Fc | Q347R/K360E/D399V/F405T/K409W | — | — | 172 | Reference sample |
| 377 | scFv.HL-Fc | E356K/K392D/D399K/K439E | — | — | 173 | Reference sample |
| 378 | scFv.HL-Fc | T394W/F405A | — | — | 174 | Reference sample |
| 379 | scFv.HL-Fc | T366W/L368A/Y407V | — | — | 175 | Reference sample |

### [Example 12] Further Search for Modifications Promoting Heavy-Chain Homodimer Formation

The above Examples revealed that the amino acid modifications in Table 4 have particularly strong heavy-chain homodimerization-promoting ability, and have wide applicability regardless of antibody subclasses, formats, and Fc modifications in template antibodies. However, amino acid modifications surrounding these effective modification sets have not been fully searched yet, and there may be other effective modifications than the modification sets of Table 4. Thus, for the modifications of T394W/F405A (T394F/F405A), T366W/L368A/Y407V, Q347R/K360E/D399V/F405T/K409W, E345R/Q347R/K360D/T366V/D399M/Y407A/K409V, E356K/D399K/K409E/K439E, E356K/K392D/D399K/K409D, E356K/K392D/D399K/K409D, K392D/D399K/K409D, and E356K/K392D/D399K/K439E in Table 4, large-scale amino acid modification was performed on each modification position. Furthermore, their heavy-chain homodimer formation ability was evaluated by SEC analysis. The correspondences between the categories of effect of the modifications used for antibody expression, amino acid modifications, SEQ ID NOs, and area ratios are shown in Table 15 (the non-introduction of modifications is denoted as “−”). Antibody expression and analysis were performed according to the methods described in Example 1. Homodimers containing each Fc modification were prepared according to the method described in Example 3, and the monomer ratio, Tm, and yield (mg) in SEC analysis were evaluated by similar methods and are shown together in Table 15. As a result, modifications for which the heteromultimer (%) in Table 15 was lower than that of the control, 51.7%, were evaluated as effective modifications. These effective modifications at each position may also be able to be used in combination.

In this test, for samples with a low monomer ratio, a half antibody derived from the homodimer (a monomer composed of one heavy chain and one light chain) was contained in the peak of heteromultimer in the homodimer analysis. Therefore, the apparent ratio of heteromultimer in these samples may be higher than the true value. Nevertheless, since this was considered not to affect the determination of amino acid modifications with strong homodimerization ability and good physical properties, the single criterion of 51.7% or lower was used to evaluate the presence or absence of the ability to suppress heteromultimer formation.

Modifications that showed a heteromultimer ratio (%) of 10% or lower in Table 15 and were therefore viewed as more effective are shown in Table 16. Further, modifications that showed a heteromultimer ratio (%) of 0% in Table 15 and were therefore viewed as more effective are shown in Table 17. Furthermore, modifications that showed a heteromultimer ratio (%) of 0% in Table 15 and for which the monomer ratio, Tm, and yield (mg) upon preparation of the homodimer were 95% or higher, 65° C. or higher, and 0.15 mg or higher in SEC analysis, respectively, and which are therefore particularly advantageous in terms of the heavy-chain homodimer formation ability and the physicochemical properties of the homodimer, are shown in Table 18. Meanwhile, the samples in Table 4 whose heteromultimer ratio (%) is 0% and for which the monomer ratio, Tm, and yield (mg) of the homodimer are 95% or higher, 65° C. or higher, and 0.15 mg or higher, respectively, are Samples Nos. 80, 87, 92, 95, 97, 100, 102, 109, 111, 119, and 121. The modifications contained in these also have a superior heavy-chain homodimer formation ability and physicochemical properties.

The amino acid modifications and their combinations at the respective positions that have been shown to be effective in Tables 15-18 are expected to have similar properties, and therefore can be used as alternatives for the modification sets of T394W/F405A (T394F/F405A), T366W/L368A/Y407V, Q347R/K360E/D399V/F405T/K409W, E345R/Q347R/K360D/T366V/D399M/Y407A/K409V, E356K/D399K/K409E/K439E, E356K/K392D/D399K/K409D, K392D/D399K/K409D, and E356K/K392D/D399K/K439E shown in Tables 6 and 7, and may be applicable as Fc modifications for expressing multiple homodimers.

In addition, in each combination of modifications, amino acids that have shown to be effective at the same modified positions may also be able to be used in combination. Combinations of modifications that showed strong suppression of heteromeric association in Table 1 and amino acid modifications in Table 15 that suppressed the heteromultimer ratio below that of WT, 51.7%, are shown in Table 19. For example, for Modification Set No. 1, the effect was present with W, F, Y, and H at EU numbering position 394 and A, S, T, C, V, and G at position 405. Thus, 4×6=24 combinations of the modifications at the respective positions are also presumed to exert a certain effect. For the other Modification Sets, similarly, the combinations of the modifications shown in the table may also exert a certain heteromultimer-suppressing ability.

Similarly, amino acid modifications that suppressed the heteromultimer ratio to 10% or lower in Table 15 are shown in Table 20. Combinations of these respective amino acids may be more preferable heteromultimer-suppressing modifications.

Furthermore, amino acid modifications that suppressed the heteromultimer ratio to 0% in Table 15 are shown in Table 21. Combinations of these respective amino acids may be even more preferable heteromultimer-suppressing modifications.

TABLE 15
Results of analysis of the homodimer formation ability and physicochemical properties of antibodies with introduced amino acid modifications

| | | | Full- | | | Homo | | | | Full- | | | | |
|---|---|---|---|---|---|---|---|---|---|---|---|---|---|---|
| | | | length | | | full- | | | | length | Light | | | |
| | | Modifications that | heavy | Light | Fc | length | | Homo | | heavy | chain | | | |
| | Category of | promote formation | chain | chain | fragment | heavy | Hetero- | Fc | | chain | SEQ | SEC | | |
| Sample | modification | of heavy chain | SEQ ID | SEQ ID | SEQ ID | chain | multimer | fragment | Sample | SEQ | ID | monomer | Tm | Yield |
| No. | effect | homodimers | NO | NO | NO | (%) | (%) | (%) | No. | ID NO | NO | ratio (%) | (° C.) | (mg) |
| 1 | WT-IgG1 | — | 1 | 61 | 62 | 31.5 | 51.7 | 16.8 | 765 | 1 | 61 | 98.19 | 68.4 | 0.23 |
| 380 | Charge | E356R/D399K/K409E/ | 176 | 61 | 62 | 61.1 | 0.0 | 38.9 | 766 | 176 | 61 | 98.02 | 63.6 | 0.22 |
| | | K439E | | | | | | | | | | | | |
| 381 | Charge | E356H/D399K/K409E/ | 177 | 61 | 62 | 56.3 | 3.3 | 40.4 | 767 | 177 | 61 | 97.16 | 61.8 | 0.22 |
| | | K439E | | | | | | | | | | | | |
| 382 | Charge | E356K/D399R/K409E/ | 178 | 61 | 62 | 61.8 | 0.0 | 38.2 | 768 | 178 | 61 | 98.83 | 51 | 0.23 |
| | | K439E | | | | | | | | | | | | |
| 383 | Charge | E356K/D399H/K409E/ | 179 | 61 | 62 | 63.8 | 0.0 | 36.2 | 769 | 179 | 61 | 97.25 | 66 | 0.15 |
| | | K439E | | | | | | | | | | | | |
| 384 | Charge | E356K/D399K/K409D/ | 180 | 61 | 62 | 64.3 | 0.0 | 35.7 | 770 | 180 | 61 | 97.73 | 63.6 | 0.15 |
| | | K439E | | | | | | | | | | | | |
| 385 | Charge | E356K/D399K/K409E/ | 181 | 61 | 62 | 57.8 | 0.0 | 42.2 | 771 | 181 | 61 | 98.88 | 64.8 | 0.23 |
| | | K439D | | | | | | | | | | | | |
| 386 | Charge | E356K/D399R/K409D/ | 182 | 61 | 62 | 58.7 | 0.0 | 41.3 | 772 | 182 | 61 | 99.1 | 66 | 0.24 |
| | | K439E | | | | | | | | | | | | |
| 387 | Charge | E356K/D399H/K409D/ | 183 | 61 | 62 | 56.4 | 1.9 | 41.7 | 773 | 183 | 61 | 99.02 | 66 | 0.24 |
| | | K439E | | | | | | | | | | | | |
| 388 | Charge | E356R/K392D/D399K/ | 184 | 61 | 62 | 59.7 | 2.6 | 37.7 | 774 | 184 | 61 | 98.74 | 67.8 | 0.2 |
| | | K439E | | | | | | | | | | | | |
| 389 | Charge | E356K/K392D/D399K/ | 185 | 61 | 62 | 56.2 | 2.4 | 41.4 | 775 | 185 | 61 | 98.66 | 67.2 | 0.24 |
| | | K439D | | | | | | | | | | | | |
| 390 | Charge | E356K/K392D/D399R/ | 186 | 61 | 62 | 57.6 | 2.5 | 39.8 | 776 | 186 | 61 | 98.79 | 69 | 0.22 |
| | | K439E | | | | | | | | | | | | |
| 391 | Charge | E356K/K392E/D399K/ | 187 | 61 | 62 | 56.5 | 2.4 | 41.0 | 777 | 187 | 61 | 98.92 | 68.4 | 0.24 |
| | | K439E | | | | | | | | | | | | |
| 392 | Charge | K392D/D399R/K409D | 188 | 61 | 62 | 56.5 | 1.8 | 41.6 | 778 | 188 | 61 | 98.97 | 65.4 | 0.22 |
| 393 | Charge | K392D/D399K/K409E | 189 | 61 | 62 | 60.0 | 0.0 | 40.0 | 779 | 189 | 61 | 98.94 | 66.6 | 0.21 |
| 394 | Charge | K392E/D399K/K409D | 190 | 61 | 62 | 54.5 | 1.9 | 43.6 | 780 | 190 | 61 | 99.27 | 64.2 | 0.24 |
| 395 | Charge | E356K/K392D/D399K/ | 191 | 61 | 62 | 57.7 | 0.0 | 42.3 | 781 | 191 | 61 | 99.17 | 64.2 | 0.24 |
| | | K409E | | | | | | | | | | | | |
| 396 | Charge | E356K/K392D/D399R/ | 192 | 61 | 62 | 59.6 | 0.0 | 40.4 | 782 | 192 | 61 | 99.32 | 62.4 | 0.21 |
| | | K409D | | | | | | | | | | | | |
| 397 | Charge | E356R/K392D/D399K/ | 193 | 61 | 62 | 61.1 | 0.0 | 38.9 | 783 | 193 | 61 | 99.4 | 61.2 | 0.2 |
| | | K409D | | | | | | | | | | | | |
| 398 | Charge | E356H/K392D/D399K/ | 194 | 61 | 62 | 59.4 | 0.0 | 40.6 | 784 | 194 | 61 | 99.16 | 61.8 | 0.23 |
| | | K409D | | | | | | | | | | | | |
| 399 | Charge | E356K/K392E/D399K/ | 195 | 61 | 62 | 59.5 | 0.0 | 40.5 | 785 | 195 | 61 | 97.87 | 61.8 | 0.21 |
| | | K409D | | | | | | | | | | | | |
| 400 | Charge | E356R/K392D/D399K/ | 196 | 61 | 62 | 57.0 | 2.7 | 40.3 | 786 | 196 | 61 | 98.69 | 66.6 | 0.21 |
| | | K439D | | | | | | | | | | | | |
| 401 | Charge | E356K/K392D/D399H/ | 197 | 61 | 62 | 61.2 | 0.0 | 38.8 | 787 | 197 | 61 | 98.75 | 62.4 | 0.18 |
| | | K409D | | | | | | | | | | | | |
| 402 | Charge | E356R/D399K/K409E/ | 198 | 61 | 62 | 56.7 | 0.0 | 43.3 | 788 | 198 | 61 | 98.84 | 63.6 | 0.21 |
| | | K439D | | | | | | | | | | | | |
| 403 | Charge | E356H/D399K/K409E/ | 199 | 61 | 62 | 54.9 | 2.3 | 42.7 | 789 | 199 | 61 | 98.33 | 61.2 | 0.25 |
| | | K439D | | | | | | | | | | | | |
| 404 | Steric hindrance + | Q347R/K360D/T366V/ | 200 | 61 | 62 | 70.6 | 0.0 | 29.4 | 790 | 200 | 61 | 94.56 | 68.4 | 0.09 |
| | Charge | D399M/Y407A/K409V | | | | | | | | | | | | |
| 405 | Steric hindrance + | E345R/Q347K/K360D/ | 201 | 61 | 62 | 68.5 | 0.0 | 31.5 | 791 | 201 | 61 | 97.44 | 68.4 | 0.15 |
| | Charge | T366V/D399M/Y407A/ | | | | | | | | | | | | |
| | | K409V | | | | | | | | | | | | |
| 406 | Steric hindrance + | E345R/Q347R/K360E/ | 202 | 61 | 62 | 65.2 | 0.0 | 34.8 | 792 | 202 | 61 | 97.72 | 68.4 | 0.19 |
| | Charge | T366V/D399M/Y407A/ | | | | | | | | | | | | |
| | | K409V | | | | | | | | | | | | |
| 407 | Steric hindrance + | Q347K/K360D/T366V/ | 203 | 61 | 62 | 63.8 | 0.0 | 36.2 | 793 | 203 | 61 | 97.64 | 68.4 | 0.19 |
| | Charge | D399M/Y407A/K409V | | | | | | | | | | | | |
| 408 | Steric hindrance + | Q347R/K360E/T366V/ | 204 | 61 | 62 | 64.0 | 0.0 | 36.0 | 794 | 204 | 61 | 97.39 | 68.4 | 0.18 |
| | Charge | D399M/Y407A/K409V | | | | | | | | | | | | |
| 409 | Steric hindrance + | Q347K/K360E/T366V/ | 205 | 61 | 62 | 66.6 | 0.0 | 33.4 | 795 | 205 | 61 | 97.43 | 68.4 | 0.18 |
| | Charge | D399M/Y407A/K409V | | | | | | | | | | | | |
| 410 | Steric hindrance + | E345R/Q347R/K360D/ | 206 | 61 | 62 | 64.4 | 0.3 | 35.3 | 796 | 206 | 61 | 97.83 | 66 | 0.21 |
| | Charge | T366V/D399Q/Y407A/ | | | | | | | | | | | | |
| | | K409Q | | | | | | | | | | | | |
| 411 | Steric hindrance + | E345R/Q347R/K360D/ | 207 | 61 | 62 | 65.7 | 0.0 | 34.3 | 797 | 207 | 61 | 96.8 | 67.2 | 0.1 |
| | Charge | T366V/D399N/Y407A/ | | | | | | | | | | | | |
| | | K409Q | | | | | | | | | | | | |
| 412 | Steric hindrance + | E345R/Q347R/K360D/ | 208 | 61 | 62 | 63.7 | 0.0 | 36.3 | 798 | 208 | 61 | 98.57 | 68.4 | 0.21 |
| | Charge | T366V/D399H/Y407A/ | | | | | | | | | | | | |
| | | K409Q | | | | | | | | | | | | |
| 413 | Steric hindrance + | E345R/Q347R/K360D/ | 209 | 61 | 62 | 64.1 | 0.0 | 35.9 | 799 | 209 | 61 | 98.96 | 65.4 | 0.21 |
| | Charge | T366V/D399I/Y407A/ | | | | | | | | | | | | |
| | | K409Q | | | | | | | | | | | | |
| 414 | Steric hindrance + | E345R/Q347R/K360D/ | 210 | 61 | 62 | 64.1 | 0.0 | 35.9 | 800 | 210 | 61 | 98.2 | 63 | 0.19 |
| | Charge | T366V/D399F/Y407A/ | | | | | | | | | | | | |
| | | K409Q | | | | | | | | | | | | |
| 415 | Steric hindrance + | E345R/Q347R/K360D/ | 211 | 61 | 62 | 65.9 | 0.0 | 34.1 | 801 | 211 | 61 | 98.36 | 63.6 | 0.19 |
| | Charge | T366V/D399Y/Y407A/ | | | | | | | | | | | | |
| | | K409Q | | | | | | | | | | | | |
| 416 | Steric hindrance + | E345R/Q347R/K360D/ | 212 | 61 | 62 | 67.8 | 0.0 | 32.2 | 802 | 212 | 61 | 98.38 | 68.4 | 0.19 |
| | Charge | T366V/D399T/Y407A/ | | | | | | | | | | | | |
| | | K409Q | | | | | | | | | | | | |
| 417 | Steric hindrance + | E345R/Q347R/K360D/ | 213 | 61 | 62 | 65.2 | 0.0 | 34.8 | 803 | 213 | 61 | 98.76 | 68.4 | 0.22 |
| | Charge | T366V/D399S/Y407A/ | | | | | | | | | | | | |
| | | K409Q | | | | | | | | | | | | |
| 418 | Steric hindrance + | E345R/Q347R/K360D/ | 214 | 61 | 62 | 36.0 | 24.9 | 39.1 | 804 | 214 | 61 | 56.71 | 54 | 0.22 |
| | Charge | T366V/D399Q/Y407A/ | | | | | | | | | | | | |
| | | K409N | | | | | | | | | | | | |
| 419 | Steric hindrance + | E345R/Q347R/K360D/ | 215 | 61 | 62 | 22.0 | 27.9 | 50.0 | 805 | 215 | 61 | 55.72 | 50.4 | 0.2 |
| | Charge | T366V/D399H/Y407A/ | | | | | | | | | | | | |
| | | K409N | | | | | | | | | | | | |
| 420 | Steric hindrance + | E345R/Q347R/K360D/ | 216 | 61 | 62 | 46.6 | 12.7 | 40.7 | 806 | 216 | 61 | 80.31 | 55.2 | 0.2 |
| | Charge | T366V/D399V/Y407A/ | | | | | | | | | | | | |
| | | K409N | | | | | | | | | | | | |
| 421 | Steric hindrance + | E345R/Q347R/K360D/ | 217 | 61 | 62 | 50.6 | 11.1 | 38.3 | 807 | 217 | 61 | 82.56 | 51.6 | 0.18 |
| | Charge | T366V/D399L/Y407A/ | | | | | | | | | | | | |
| | | K409N | | | | | | | | | | | | |
| 422 | Steric hindrance + | E345R/Q347R/K360D/ | 218 | 61 | 62 | 51.5 | 4.2 | 44.2 | 808 | 218 | 61 | 93.41 | 50.4 | 0.2 |
| | Charge | T366V/D399F/Y407A/ | | | | | | | | | | | | |
| | | K409N | | | | | | | | | | | | |
| 423 | Steric hindrance + | E345R/Q347R/K360D/ | 219 | 61 | 62 | 31.9 | 16.3 | 51.9 | 809 | 219 | 61 | 74.87 | 62.4 | 0.18 |
| | Charge | T366V/D399Y/Y407A/ | | | | | | | | | | | | |
| | | K409N | | | | | | | | | | | | |
| 424 | Steric hindrance + | E345R/Q347R/K360D/ | 220 | 61 | 62 | 48.6 | 13.7 | 37.6 | 810 | 220 | 61 | 79.22 | 53.4 | 0.23 |
| | Charge | T366V/D399T/Y407A/ | | | | | | | | | | | | |
| | | K409N | | | | | | | | | | | | |
| 425 | Steric hindrance + | E345R/Q347R/K360D/ | 221 | 61 | 62 | 28.4 | 27.6 | 44.0 | 811 | 221 | 61 | 50.49 | 52.2 | 0.23 |
| | Charge | T366V/D399S/Y407A/ | | | | | | | | | | | | |
| | | K409N | | | | | | | | | | | | |
| 426 | Steric hindrance + | E345R/Q347R/K360D/ | 222 | 61 | 62 | 57.9 | 1.7 | 40.4 | 812 | 222 | 61 | 96.33 | 65.4 | 0.22 |
| | Charge | T366V/D399Q/Y407A/ | | | | | | | | | | | | |
| | | K409H | | | | | | | | | | | | |
| 427 | Steric hindrance + | E345R/Q347R/K360D/ | 223 | 61 | 62 | 59.2 | 1.1 | 39.7 | 813 | 223 | 61 | 97.13 | 64.8 | 0.21 |
| | Charge | T366V/D399N/Y407A/ | | | | | | | | | | | | |
| | | K409H | | | | | | | | | | | | |
| 428 | Steric hindrance + | E345R/Q347R/K360D/ | 224 | 61 | 62 | 59.3 | 1.5 | 39.2 | 814 | 224 | 61 | 96.11 | 64.2 | 0.21 |
| | Charge | T366V/D399H/Y407A/ | | | | | | | | | | | | |
| | | K409H | | | | | | | | | | | | |
| 429 | Steric hindrance + | E345R/Q347R/K360D/ | 225 | 61 | 62 | 65.7 | 0.0 | 34.3 | 815 | 225 | 61 | 98.81 | 67.2 | 0.2 |
| | Charge | T366V/D399V/Y407A/ | | | | | | | | | | | | |
| | | K409H | | | | | | | | | | | | |
| 430 | Steric hindrance + | E345R/Q347R/K360D/ | 226 | 61 | 62 | 60.7 | 0.0 | 39.3 | 816 | 226 | 61 | 98.22 | 64.8 | 0.21 |
| | Charge | T366V/D399L/Y407A/ | | | | | | | | | | | | |
| | | K409H | | | | | | | | | | | | |
| 431 | Steric hindrance + | E345R/Q347R/K360D/ | 227 | 61 | 62 | 59.3 | 1.4 | 39.3 | 817 | 227 | 61 | 95.94 | 59.4 | 0.22 |
| | Charge | T366V/D399F/Y407A/ | | | | | | | | | | | | |
| | | K409H | | | | | | | | | | | | |
| 432 | Steric hindrance + | E345R/Q347R/K360D/ | 228 | 61 | 62 | 59.9 | 1.6 | 38.5 | 818 | 228 | 61 | 96.08 | 60.6 | 0.18 |
| | Charge | T366V/D399Y/Y407A/ | | | | | | | | | | | | |
| | | K409H | | | | | | | | | | | | |
| 433 | Steric hindrance + | E345R/Q347R/K360D/ | 229 | 61 | 62 | 65.1 | 0.0 | 34.9 | 819 | 229 | 61 | 98.64 | 67.8 | 0.2 |
| | Charge | T366V/D399T/Y407A/ | | | | | | | | | | | | |
| | | K409H | | | | | | | | | | | | |
| 434 | Steric hindrance + | E345R/Q347R/K360D/ | 230 | 61 | 62 | 62.2 | 0.0 | 37.8 | 820 | 230 | 61 | 98.28 | 67.8 | 0.2 |
| | Charge | T366V/D399S/Y407A/ | | | | | | | | | | | | |
| | | K409H | | | | | | | | | | | | |
| 435 | Steric hindrance + | E345R/Q347R/K360D/ | 231 | 61 | 62 | 64.5 | 0.0 | 35.5 | 821 | 231 | 61 | 98.23 | 69 | 0.2 |
| | Charge | T366V/D399Q/Y407A/ | | | | | | | | | | | | |
| | | K409V | | | | | | | | | | | | |
| 436 | Steric hindrance + | E345R/Q347R/K360D/ | 232 | 61 | 62 | 65.4 | 0.0 | 34.6 | 822 | 232 | 61 | 98.41 | 68.4 | 0.2 |
| | Charge | T366V/D399N/Y407A/ | | | | | | | | | | | | |
| | | K409V | | | | | | | | | | | | |
| 437 | Steric hindrance + | E345R/Q347R/K360D/ | 233 | 61 | 62 | 62.6 | 0.0 | 37.4 | 823 | 233 | 61 | 98.66 | 67.8 | 0.2 |
| | Charge | T366V/D399H/Y407A/ | | | | | | | | | | | | |
| | | K409V | | | | | | | | | | | | |
| 438 | Steric hindrance + | E345R/Q347R/K360D/ | 234 | 61 | 62 | 64.3 | 0.0 | 35.7 | 824 | 234 | 61 | 98.42 | 68.4 | 0.22 |
| | Charge | T366V/D399V/Y407A/ | | | | | | | | | | | | |
| | | K409V | | | | | | | | | | | | |
| 439 | Steric hindrance + | E345R/Q347R/K360D/ | 235 | 61 | 62 | 61.7 | 0.0 | 38.3 | 825 | 235 | 61 | 97.67 | 67.2 | 0.2 |
| | Charge | T366V/D399L/Y407A/ | | | | | | | | | | | | |
| | | K409V | | | | | | | | | | | | |
| 440 | Steric hindrance + | E345R/Q347R/K360D/ | 236 | 61 | 62 | 62.9 | 0.0 | 37.1 | 826 | 236 | 61 | 98.16 | 68.4 | 0.21 |
| | Charge | T366V/D399I/Y407A/ | | | | | | | | | | | | |
| | | K409V | | | | | | | | | | | | |
| 441 | Steric hindrance + | E345R/Q347R/K360D/ | 237 | 61 | 62 | 62.6 | 0.0 | 37.4 | 827 | 237 | 61 | 98.26 | 65.4 | 0.2 |
| | Charge | T366V/D399F/Y407A/ | | | | | | | | | | | | |
| | | K409V | | | | | | | | | | | | |
| 442 | Steric hindrance + | E345R/Q347R/K360D/ | 238 | 61 | 62 | 63.7 | 0.0 | 36.3 | 828 | 238 | 61 | 98.46 | 67.2 | 0.21 |
| | Charge | T366V/D399Y/Y407A/ | | | | | | | | | | | | |
| | | K409V | | | | | | | | | | | | |
| 443 | Steric hindrance + | E345R/Q347R/K360D/ | 239 | 61 | 62 | 65.2 | 0.0 | 34.8 | 829 | 239 | 61 | 98.22 | 69 | 0.19 |
| | Charge | T366V/D399T/Y407A/ | | | | | | | | | | | | |
| | | K409V | | | | | | | | | | | | |
| 444 | Steric hindrance + | E345R/Q347R/K360D/ | 240 | 61 | 62 | 66.1 | 0.0 | 33.9 | 830 | 240 | 61 | 98.19 | 69 | 0.18 |
| | Charge | T366V/D399S/Y407A/ | | | | | | | | | | | | |
| | | K409V | | | | | | | | | | | | |
| 445 | Steric hindrance + | E345R/Q347R/K360D/ | 241 | 61 | 62 | 66.9 | 0.0 | 33.1 | 831 | 241 | 61 | 98.01 | 69 | 0.19 |
| | Charge | T366V/D399Q/Y407A/ | | | | | | | | | | | | |
| | | K409L | | | | | | | | | | | | |
| 446 | Steric hindrance + | E345R/Q347R/K360D/ | 242 | 61 | 62 | 61.5 | 0.0 | 38.5 | 832 | 242 | 61 | 98.01 | 68.4 | 0.18 |
| | Charge | T366V/D399N/Y407A/ | | | | | | | | | | | | |
| | | K409L | | | | | | | | | | | | |
| 447 | Steric hindrance + | E345R/Q347R/K360D/ | 243 | 61 | 62 | 62.2 | 0.0 | 37.8 | 833 | 243 | 61 | 98.41 | 67.8 | 0.2 |
| | Charge | T366V/D399H/Y407A/ | | | | | | | | | | | | |
| | | K409L | | | | | | | | | | | | |
| 448 | Steric hindrance + | E345R/Q347R/K360D/ | 244 | 61 | 62 | 61.5 | 0.0 | 38.5 | 834 | 244 | 61 | 97.25 | 67.8 | 0.19 |
| | Charge | T366V/D399I/Y407A/ | | | | | | | | | | | | |
| | | K409L | | | | | | | | | | | | |
| 449 | Steric hindrance + | E345R/Q347R/K360D/ | 245 | 61 | 62 | 60.9 | 0.0 | 39.1 | 835 | 245 | 61 | 97.83 | 66 | 0.19 |
| | Charge | T366V/D399F/Y407A/ | | | | | | | | | | | | |
| | | K409L | | | | | | | | | | | | |
| 450 | Steric hindrance + | E345R/Q347R/K360D/ | 246 | 61 | 62 | 61.2 | 0.0 | 38.8 | 836 | 246 | 61 | 97.48 | 69 | 0.21 |
| | Charge | T366V/D399T/Y407A/ | | | | | | | | | | | | |
| | | K409L | | | | | | | | | | | | |
| 451 | Steric hindrance + | E345R/Q347R/K360D/ | 247 | 61 | 62 | 62.2 | 0.0 | 37.8 | 837 | 247 | 61 | 98.08 | 69.6 | 0.21 |
| | Charge | T366V/D399S/Y407A/ | | | | | | | | | | | | |
| | | K409L | | | | | | | | | | | | |
| 452 | Steric hindrance + | E345R/Q347R/K360D/ | 248 | 61 | 62 | 66.2 | 0.0 | 33.8 | 838 | 248 | 61 | 97.82 | 68.4 | 0.17 |
| | Charge | T366V/D399Q/Y407A/ | | | | | | | | | | | | |
| | | K409I | | | | | | | | | | | | |
| 453 | Steric hindrance + | E345R/Q347R/K360D/ | 249 | 61 | 62 | 63.0 | 0.0 | 37.0 | 839 | 249 | 61 | 98.15 | 68.4 | 0.17 |
| | Charge | T366V/D399N/Y407A/ | | | | | | | | | | | | |
| | | K409I | | | | | | | | | | | | |
| 454 | Steric hindrance + | E345R/Q347R/K360D/ | 250 | 61 | 62 | 62.1 | 0.0 | 37.9 | 840 | 250 | 61 | 98.95 | 67.2 | 0.21 |
| | Charge | T366V/D399H/Y407A/ | | | | | | | | | | | | |
| | | K409I | | | | | | | | | | | | |
| 455 | Steric hindrance + | E345R/Q347R/K360D/ | 251 | 61 | 62 | 65.1 | 0.0 | 34.9 | 841 | 251 | 61 | 97.95 | 68.4 | 0.21 |
| | Charge | T366V/D399V/Y407A/ | | | | | | | | | | | | |
| | | K409I | | | | | | | | | | | | |
| 456 | Steric hindrance + | E345R/Q347R/K360D/ | 252 | 61 | 62 | 59.0 | 0.0 | 41.0 | 842 | 252 | 61 | 97.02 | 65.4 | 0.17 |
| | Charge | T366V/D399L/Y407A/ | | | | | | | | | | | | |
| | | K409I | | | | | | | | | | | | |
| 457 | Steric hindrance + | E345R/Q347R/K360D/ | 253 | 61 | 62 | 61.0 | 0.0 | 39.0 | 843 | 253 | 61 | 98.53 | 64.8 | 0.21 |
| | Charge | T366V/D399F/Y407A/ | | | | | | | | | | | | |
| | | K409I | | | | | | | | | | | | |
| 458 | Steric hindrance + | E345R/Q347R/K360D/ | 254 | 61 | 62 | 62.4 | 0.0 | 37.6 | 844 | 254 | 61 | 98.49 | 67.2 | 0.21 |
| | Charge | T366V/D399Y/Y407A/ | | | | | | | | | | | | |
| | | K409I | | | | | | | | | | | | |
| 459 | Steric hindrance + | E345R/Q347R/K360D/ | 255 | 61 | 62 | 60.5 | 0.0 | 39.5 | 845 | 255 | 61 | 98.24 | 69 | 0.21 |
| | Charge | T366V/D399T/Y407A/ | | | | | | | | | | | | |
| | | K409I | | | | | | | | | | | | |
| 460 | Steric hindrance + | E345R/Q347R/K360D/ | 256 | 61 | 62 | 65.6 | 0.0 | 34.4 | 846 | 256 | 61 | 97.66 | 69 | 0.16 |
| | Charge | T366V/D399S/Y407A/ | | | | | | | | | | | | |
| | | K409I | | | | | | | | | | | | |
| 461 | Steric hindrance + | E345R/Q347R/K360D/ | 257 | 61 | 62 | 63.4 | 0.0 | 36.6 | 847 | 257 | 61 | 98.6 | 67.2 | 0.21 |
| | Charge | T366V/D399Q/Y407A/ | | | | | | | | | | | | |
| | | K409F | | | | | | | | | | | | |
| 462 | Steric hindrance + | E345R/Q347R/K360D/ | 258 | 61 | 62 | 65.9 | 0.0 | 34.1 | 848 | 258 | 61 | 98.68 | 66.6 | 0.2 |
| | Charge | T366V/D399N/Y407A/ | | | | | | | | | | | | |
| | | K409F | | | | | | | | | | | | |
| 463 | Steric hindrance + | E345R/Q347R/K360D/ | 259 | 61 | 62 | 60.5 | 0.0 | 39.5 | 849 | 259 | 61 | 98.77 | 67.2 | 0.2 |
| | Charge | T366V/D399H/Y407A/ | | | | | | | | | | | | |
| | | K409F | | | | | | | | | | | | |
| 464 | Steric hindrance + | E345R/Q347R/K360D/ | 260 | 61 | 62 | 62.3 | 0.0 | 37.7 | 850 | 260 | 61 | 98.54 | 67.2 | 0.21 |
| | Charge | T366V/D399V/Y407A/ | | | | | | | | | | | | |
| | | K409F | | | | | | | | | | | | |
| 465 | Steric hindrance + | E345R/Q347R/K360D/ | 261 | 61 | 62 | 59.8 | 0.0 | 40.2 | 851 | 261 | 61 | 98.63 | 66 | 0.18 |
| | Charge | T366V/D399L/Y407A/ | | | | | | | | | | | | |
| | | K409F | | | | | | | | | | | | |
| 466 | Steric hindrance + | E345R/Q347R/K360D/ | 262 | 61 | 62 | 61.5 | 0.0 | 38.5 | 852 | 262 | 61 | 98.5 | 66 | 0.22 |
| | Charge | T366V/D399I/Y407A/ | | | | | | | | | | | | |
| | | K409F | | | | | | | | | | | | |
| 467 | Steric hindrance + | E345R/Q347R/K360D/ | 263 | 61 | 62 | 60.3 | 0.0 | 39.7 | 853 | 263 | 61 | 98.73 | 63 | 0.19 |
| | Charge | T366V/D399F/Y407A/ | | | | | | | | | | | | |
| | | K409F | | | | | | | | | | | | |
| 468 | Steric hindrance + | E345R/Q347R/K360D/ | 264 | 61 | 62 | 66.0 | 0.0 | 34.0 | 854 | 264 | 61 | 98.86 | 64.8 | 0.19 |
| | Charge | T366V/D399Y/Y407A/ | | | | | | | | | | | | |
| | | K409F | | | | | | | | | | | | |
| 469 | Steric hindrance + | E345R/Q347R/K360D/ | 265 | 61 | 62 | 64.2 | 0.0 | 35.8 | 855 | 265 | 61 | 98.56 | 67.2 | 0.22 |
| | Charge | T366V/D399T/Y407A/ | | | | | | | | | | | | |
| | | K409F | | | | | | | | | | | | |
| 470 | Steric hindrance + | E345R/Q347R/K360D/ | 266 | 61 | 62 | 59.8 | 1.8 | 38.5 | 856 | 266 | 61 | 95.94 | 64.8 | 0.24 |
| | Charge | T366V/D399Q/Y407A/ | | | | | | | | | | | | |
| | | K409Y | | | | | | | | | | | | |
| 471 | Steric hindrance + | E345R/Q347R/K360D/ | 267 | 61 | 62 | 59.3 | 1.4 | 39.2 | 857 | 267 | 61 | 96 | 64.2 | 0.13 |
| | Charge | T366V/D399H/Y407A/ | | | | | | | | | | | | |
| | | K409Y | | | | | | | | | | | | |
| 472 | Steric hindrance + | E345R/Q347R/K360D/ | 268 | 61 | 62 | 58.8 | 0.0 | 41.2 | 858 | 268 | 61 | 98.16 | 66.6 | 0.23 |
| | Charge | T366V/D399V/Y407A/ | | | | | | | | | | | | |
| | | K409Y | | | | | | | | | | | | |
| 473 | Steric hindrance + | E345R/Q347R/K360D/ | 269 | 61 | 62 | 57.7 | 0.0 | 42.3 | 859 | 269 | 61 | 96.91 | 64.2 | 0.21 |
| | Charge | T366V/D399L/Y407A/ | | | | | | | | | | | | |
| | | K409Y | | | | | | | | | | | | |
| 474 | Steric hindrance + | E345R/Q347R/K360D/ | 270 | 61 | 62 | 65.9 | 0.0 | 34.1 | 860 | 270 | 61 | 97.86 | 64.8 | 0.19 |
| | Charge | T366V/D399I/Y407A/ | | | | | | | | | | | | |
| | | K409Y | | | | | | | | | | | | |
| 475 | Steric hindrance + | E345R/Q347R/K360D/ | 271 | 61 | 62 | 59.8 | 0.0 | 40.2 | 861 | 271 | 61 | 97.35 | 60.6 | 0.18 |
| | Charge | T366V/D399F/Y407A/ | | | | | | | | | | | | |
| | | K409Y | | | | | | | | | | | | |
| 476 | Steric hindrance + | E345R/Q347R/K360D/ | 272 | 61 | 62 | 66.7 | 1.3 | 32.1 | 862 | 272 | 61 | 96.17 | 61.8 | 0.15 |
| | Charge | T366V/D399Y/Y407A/ | | | | | | | | | | | | |
| | | K409Y | | | | | | | | | | | | |
| 477 | Steric hindrance + | E345R/Q347R/K360D/ | 273 | 61 | 62 | 64.6 | 0.0 | 35.4 | 863 | 273 | 61 | 97.16 | 66 | 0.21 |
| | Charge | T366V/D399S/Y407A/ | | | | | | | | | | | | |
| | | K409Y | | | | | | | | | | | | |
| 478 | Steric hindrance + | E345R/Q347R/K360D/ | 274 | 61 | 62 | 66.7 | 0.0 | 33.3 | 864 | 274 | 61 | 97.13 | 67.2 | 0.19 |
| | Charge | T366V/D399N/Y407A/ | | | | | | | | | | | | |
| | | K409T | | | | | | | | | | | | |
| 479 | Steric hindrance + | E345R/Q347R/K360D/ | 275 | 61 | 62 | 61.2 | 2.1 | 36.7 | 865 | 275 | 61 | 94.75 | 65.4 | 0.19 |
| | Charge | T366V/D399H/Y407A/ | | | | | | | | | | | | |
| | | K409T | | | | | | | | | | | | |
| 480 | Steric hindrance + | E345R/Q347R/K360D/ | 276 | 61 | 62 | 63.2 | 0.0 | 36.8 | 866 | 276 | 61 | 97.89 | 67.2 | 0.23 |
| | Charge | T366V/D399V/Y407A/ | | | | | | | | | | | | |
| | | K409T | | | | | | | | | | | | |
| 481 | Steric hindrance + | E345R/Q347R/K360D/ | 277 | 61 | 62 | 68.0 | 0.0 | 32.0 | 867 | 277 | 61 | 96.8 | 51 | 0.19 |
| | Charge | T366V/D399L/Y407A/ | | | | | | | | | | | | |
| | | K409T | | | | | | | | | | | | |
| 482 | Steric hindrance + | E345R/Q347R/K360D/ | 278 | 61 | 62 | 72.4 | 0.0 | 27.6 | 868 | 278 | 61 | 95.62 | 67.2 | 0.09 |
| | Charge | T366V/D399I/Y407A/ | | | | | | | | | | | | |
| | | K409T | | | | | | | | | | | | |
| 483 | Steric hindrance + | E345R/Q347R/K360D/ | 279 | 61 | 62 | 61.6 | 1.1 | 37.3 | 869 | 279 | 61 | 96.53 | 63 | 0.22 |
| | Charge | T366V/D399F/Y407A/ | | | | | | | | | | | | |
| | | K409T | | | | | | | | | | | | |
| 484 | Steric hindrance + | E345R/Q347R/K360D/ | 280 | 61 | 62 | 62.2 | 1.1 | 36.7 | 870 | 280 | 61 | 96.78 | 64.2 | 0.22 |
| | Charge | T366V/D399Y/Y407A/ | | | | | | | | | | | | |
| | | K409T | | | | | | | | | | | | |
| 485 | Steric hindrance + | E345R/Q347R/K360D/ | 281 | 61 | 62 | 66.8 | 0.0 | 33.2 | 871 | 281 | 61 | 97.6 | 67.8 | 0.22 |
| | Charge | T366V/D399T/Y407A/ | | | | | | | | | | | | |
| | | K409T | | | | | | | | | | | | |
| 486 | Steric hindrance + | E345R/Q347R/K360D/ | 282 | 61 | 62 | 64.9 | 0.0 | 35.1 | 872 | 282 | 61 | 97.27 | 68.4 | 0.16 |
| | Charge | T366V/D399S/Y407A/ | | | | | | | | | | | | |
| | | K409T | | | | | | | | | | | | |
| 487 | Steric hindrance + | E345R/Q347R/K360D/ | 283 | 61 | 62 | 62.9 | 0.0 | 37.1 | 873 | 283 | 61 | 98.65 | 69 | 0.23 |
| | Charge | T366V/D399Q/Y407A/ | | | | | | | | | | | | |
| | | K409S | | | | | | | | | | | | |
| 488 | Steric hindrance + | E345R/Q347R/K360D/ | 284 | 61 | 62 | 61.1 | 0.0 | 38.9 | 874 | 284 | 61 | 98.77 | 67.8 | 0.24 |
| | Charge | T366V/D399N/Y407A/ | | | | | | | | | | | | |
| | | K409S | | | | | | | | | | | | |
| 489 | Steric hindrance + | E345R/Q347R/K360D/ | 285 | 61 | 62 | 60.8 | 0.0 | 39.2 | 875 | 285 | 61 | 97.45 | 65.4 | 0.24 |
| | Charge | T366V/D399H/Y407A/ | | | | | | | | | | | | |
| | | K409S | | | | | | | | | | | | |
| 490 | Steric hindrance + | E345R/Q347R/K360D/ | 286 | 61 | 62 | 64.5 | 0.0 | 35.5 | 876 | 286 | 61 | 98.58 | 67.2 | 0.21 |
| | Charge | T366V/D399V/Y407A/ | | | | | | | | | | | | |
| | | K409S | | | | | | | | | | | | |
| 491 | Steric hindrance + | E345R/Q347R/K360D/ | 287 | 61 | 62 | 60.9 | 0.0 | 39.1 | 877 | 287 | 61 | 98.74 | 67.2 | 0.22 |
| | Charge | T366V/D399L/Y407A/ | | | | | | | | | | | | |
| | | K409S | | | | | | | | | | | | |
| 492 | Steric hindrance + | E345R/Q347R/K360D/ | 288 | 61 | 62 | 60.8 | 0.0 | 39.2 | 878 | 288 | 61 | 98.89 | 66.6 | 0.23 |
| | Charge | T366V/D399I/Y407A/ | | | | | | | | | | | | |
| | | K409S | | | | | | | | | | | | |
| 493 | Steric hindrance + | E345R/Q347R/K360D | 289 | 61 | 62 | 59.9 | 0.0 | 40.1 | 879 | 289 | 61 | 98.26 | 61.8 | 0.22 |
| | Charge | T366V/D399F/Y407A/ | | | | | | | | | | | | |
| | | K409S | | | | | | | | | | | | |
| 494 | Steric hindrance + | E345R/Q347R/K360D/ | 290 | 61 | 62 | 65.7 | 0.0 | 34.3 | 880 | 290 | 61 | 97.06 | 62.4 | 0.15 |
| | Charge | T366V/D399Y/Y407A/ | | | | | | | | | | | | |
| | | K409S | | | | | | | | | | | | |
| 495 | Steric hindrance + | E345R/Q347R/K360D/ | 291 | 61 | 62 | 67.0 | 0.0 | 33.0 | 881 | 291 | 61 | 98.57 | 68.4 | 0.23 |
| | Charge | T366V/D399T/Y407A/ | | | | | | | | | | | | |
| | | K409S | | | | | | | | | | | | |
| 496 | Steric hindrance + | E345R/Q347R/K360D/ | 292 | 61 | 62 | 61.5 | 0.0 | 38.5 | 882 | 292 | 61 | 98.73 | 53.4 | 0.22 |
| | Charge | T366V/D399S/Y407A/ | | | | | | | | | | | | |
| | | K409S | | | | | | | | | | | | |
| 497 | Steric hindrance + | E345R/Q347R/K360D/ | 293 | 61 | 62 | 65.0 | 0.0 | 35.0 | 883 | 293 | 61 | 97.5 | 66.6 | 0.16 |
| | Charge | T366V/D399M/Y407A/ | | | | | | | | | | | | |
| | | K409Q | | | | | | | | | | | | |
| 498 | Steric hindrance + | E345R/Q347R/K360D/ | 294 | 61 | 62 | 47.6 | 14.3 | 38.1 | 884 | 294 | 61 | 75.67 | 55.8 | 0.22 |
| | Charge | T366V/D399M/Y407A/ | | | | | | | | | | | | |
| | | K409N | | | | | | | | | | | | |
| 499 | Steric hindrance + | E345R/Q347R/K360D/ | 295 | 61 | 62 | 54.4 | 0.7 | 44.9 | 885 | 295 | 61 | 95.32 | 65.4 | 0.22 |
| | Charge | T366V/D399M/Y407A/ | | | | | | | | | | | | |
| | | K409H | | | | | | | | | | | | |
| 500 | Steric hindrance + | E345R/Q347R/K360D/ | 296 | 61 | 62 | 59.0 | 0.0 | 41.0 | 886 | 296 | 61 | 97.5 | 68.4 | 0.22 |
| | Charge | T366V/D399M/Y407A/ | | | | | | | | | | | | |
| | | K409L | | | | | | | | | | | | |
| 501 | Steric hindrance + | E345R/Q347R/K360D/ | 297 | 61 | 62 | 59.0 | 0.0 | 41.0 | 887 | 297 | 61 | 97.7 | 67.2 | 0.23 |
| | Charge | T366V/D399M/Y407A/ | | | | | | | | | | | | |
| | | K409I | | | | | | | | | | | | |
| 502 | Steric hindrance + | E345R/Q347R/K360D/ | 298 | 61 | 62 | 64.9 | 0.0 | 35.1 | 888 | 298 | 61 | 98.14 | 66.6 | 0.22 |
| | Charge | T366V/D399M/Y407A/ | | | | | | | | | | | | |
| | | K409F | | | | | | | | | | | | |
| 503 | Steric hindrance + | E345R/Q347R/K360D/ | 299 | 61 | 62 | 60.5 | 0.0 | 39.5 | 889 | 299 | 61 | 97.03 | 65.4 | 0.22 |
| | Charge | T366V/D399M/Y407A/ | | | | | | | | | | | | |
| | | K409Y | | | | | | | | | | | | |
| 504 | Steric hindrance + | E345R/Q347R/K360D/ | 300 | 61 | 62 | 62.1 | 0.0 | 37.9 | 890 | 300 | 61 | 98.04 | 68.4 | 0.25 |
| | Charge | T366V/D399M/Y407A/ | | | | | | | | | | | | |
| | | K409T | | | | | | | | | | | | |
| 505 | Steric hindrance + | E345R/Q347R/K360D/ | 301 | 61 | 62 | 61.3 | 0.0 | 38.7 | 891 | 301 | 61 | 98.79 | 68.4 | 0.25 |
| | Charge | T366V/D399M/Y407A/ | | | | | | | | | | | | |
| | | K409S | | | | | | | | | | | | |
| 506 | Steric hindrance + | E345R/Q347R/K360D/ | 302 | 61 | 62 | 65.0 | 0.0 | 35.0 | 892 | 302 | 61 | 98.63 | 67.2 | 0.22 |
| | Charge | T366V/D399V/Y407A/ | | | | | | | | | | | | |
| | | K409Q | | | | | | | | | | | | |
| 507 | Steric hindrance + | E345R/Q347R/K360D/ | 303 | 61 | 62 | 67.2 | 0.0 | 32.8 | 893 | 303 | 61 | 97.55 | 64.2 | 0.19 |
| | Charge | T366V/D399L/Y407A/ | | | | | | | | | | | | |
| | | K409Q | | | | | | | | | | | | |
| 508 | Steric hindrance + | E345R/Q347R/K360D/ | 304 | 61 | 62 | 47.3 | 9.0 | 43.7 | 894 | 304 | 61 | 86.62 | 52.2 | 0.19 |
| | Charge | T366V/D399I/Y407A/ | | | | | | | | | | | | |
| | | K409N | | | | | | | | | | | | |
| 509 | Steric hindrance + | E345R/Q347R/K360D/ | 305 | 61 | 62 | 60.9 | 0.0 | 39.1 | 895 | 305 | 61 | 98.46 | 64.8 | 0.24 |
| | Charge | T366V/D399I/Y407A/ | | | | | | | | | | | | |
| | | K409H | | | | | | | | | | | | |
| 510 | Steric hindrance + | E345R/Q347R/K360D/ | 306 | 61 | 62 | 61.1 | 0.0 | 38.9 | 896 | 306 | 61 | 97.51 | 69 | 0.22 |
| | Charge | T366V/D399V/Y407A/ | | | | | | | | | | | | |
| | | K409L | | | | | | | | | | | | |
| 511 | Steric hindrance + | E345R/Q347R/K360D/ | 307 | 61 | 62 | 66.4 | 0.0 | 33.6 | 897 | 307 | 61 | 97.98 | 67.2 | 0.22 |
| | Charge | T366V/D399Y/Y407A/ | | | | | | | | | | | | |
| | | K409L | | | | | | | | | | | | |
| 512 | Steric hindrance + | E345R/Q347R/K360D/ | 308 | 61 | 62 | 63.9 | 0.0 | 36.1 | 898 | 308 | 61 | 98.3 | 67.8 | 0.23 |
| | Charge | T366V/D399S/Y407A/ | | | | | | | | | | | | |
| | | K409F | | | | | | | | | | | | |
| 513 | Steric hindrance + | E345R/Q347R/K360D/ | 309 | 61 | 62 | 63.4 | 0.6 | 35.9 | 899 | 309 | 61 | 96.94 | 64.8 | 0.21 |
| | Charge | T366V/D399N/Y407A/ | | | | | | | | | | | | |
| | | K409Y | | | | | | | | | | | | |
| 514 | Steric hindrance + | E345R/Q347R/K360D/ | 310 | 61 | 62 | 64.0 | 0.0 | 36.0 | 900 | 310 | 61 | 97.62 | 66.6 | 0.21 |
| | Charge | T366V/D399T/Y407A/ | | | | | | | | | | | | |
| | | K409Y | | | | | | | | | | | | |
| 515 | Steric hindrance + | E345R/Q347R/K360D/ | 311 | 61 | 62 | 64.7 | 0.0 | 35.3 | 901 | 311 | 61 | 96.89 | 67.8 | 0.23 |
| | Charge | T366V/D399Q/Y407A/ | | | | | | | | | | | | |
| | | K409T | | | | | | | | | | | | |
| 516 | Steric hindrance + | E345R/Q347R/K360D/ | 312 | 61 | 62 | 26.0 | 16.6 | 57.4 | 902 | 312 | 61 | 61.5 | 51.6 | 0.23 |
| | Charge | T366V/D399N/Y407A/ | | | | | | | | | | | | |
| | | K409N | | | | | | | | | | | | |
| 517 | Steric hindrance + | Q347R/K360E/D399V/ | 313 | 61 | 62 | 64.0 | 0.0 | 36.0 | 903 | 313 | 61 | 98.54 | 68.4 | 0.2 |
| | Charge | F405T/K409F | | | | | | | | | | | | |
| 518 | Steric hindrance + | Q347K/K360E/D399V/ | 314 | 61 | 62 | 62.0 | 0.0 | 38.0 | 904 | 314 | 61 | 98.56 | 69.6 | 0.24 |
| | Charge | F405T/K409W | | | | | | | | | | | | |
| 519 | Steric hindrance + | Q347Y/K360E/D399V/ | 315 | 61 | 62 | 61.7 | 0.0 | 38.3 | 905 | 315 | 61 | 98.39 | 68.4 | 0.25 |
| | Charge | F405T/K409W | | | | | | | | | | | | |
| 520 | Steric hindrance + | Q347R/K360D/D399V/ | 316 | 61 | 62 | 58.5 | 0.0 | 41.5 | 906 | 316 | 61 | 98.43 | 68.4 | 0.25 |
| | Charge | F405T/K409W | | | | | | | | | | | | |
| 521 | Steric hindrance + | Q347K/K360D/D399V/ | 317 | 61 | 62 | 63.2 | 0.0 | 36.8 | 907 | 317 | 61 | 97.86 | 69 | 0.22 |
| | Charge | F405T/K409W | | | | | | | | | | | | |
| 522 | Steric hindrance + | Q347H/K360D/D399V/ | 318 | 61 | 62 | 64.0 | 0.0 | 36.0 | 908 | 318 | 61 | 98.1 | 67.8 | 0.22 |
| | Charge | F405T/K409W | | | | | | | | | | | | |
| 523 | Steric hindrance + | Q347Y/K360D/D399V/ | 319 | 61 | 62 | 59.1 | 0.9 | 40.0 | 909 | 319 | 61 | 97.64 | 66.6 | 0.24 |
| | Charge | F405T/K409W | | | | | | | | | | | | |
| 524 | Steric hindrance + | Q347R/K360E/D399L/ | 320 | 61 | 62 | 58.1 | 0.0 | 41.9 | 910 | 320 | 61 | 98.62 | 69 | 0.25 |
| | Charge | F405T/K409W | | | | | | | | | | | | |
| 525 | Steric hindrance + | Q347R/K360E/D399I/ | 321 | 61 | 62 | 60.9 | 0.0 | 39.1 | 911 | 321 | 61 | 98.75 | 69 | 0.22 |
| | Charge | F405T/K409W | | | | | | | | | | | | |
| 526 | Steric hindrance + | Q347R/K360E/D399M/ | 322 | 61 | 62 | 61.9 | 0.0 | 38.1 | 912 | 322 | 61 | 98.69 | 69 | 0.23 |
| | Charge | F405T/K409W | | | | | | | | | | | | |
| 527 | Steric hindrance + | Q347R/K360E/D399S/ | 323 | 61 | 62 | 62.4 | 0.0 | 37.6 | 913 | 323 | 61 | 98.64 | 69 | 0.24 |
| | Charge | F405T/K409W | | | | | | | | | | | | |
| 528 | Steric hindrance + | Q347R/K360E/D399T/ | 324 | 61 | 62 | 61.8 | 0.0 | 38.2 | 914 | 324 | 61 | 98.56 | 69 | 0.24 |
| | Charge | F405T/K409W | | | | | | | | | | | | |
| 529 | Steric hindrance + | Q347R/K360E/D399C/ | 325 | 61 | 62 | 61.3 | 0.0 | 38.7 | 915 | 325 | 61 | 98.55 | 69.6 | 0.23 |
| | Charge + Disulfide | F405T/K409W | | | | | | | | | | | | |
| | bond | | | | | | | | | | | | | |
| 530 | Steric hindrance + | Q347R/K360E/D399H/ | 326 | 61 | 62 | 59.2 | 0.0 | 40.8 | 916 | 326 | 61 | 96.9 | 67.8 | 0.2 |
| | Charge | F405T/K409W | | | | | | | | | | | | |
| 531 | Steric hindrance + | Q347R/K360E/D399V/ | 327 | 61 | 62 | 62.5 | 0.0 | 37.5 | 917 | 327 | 61 | 98.07 | 67.8 | 0.21 |
| | Charge | F405A/K409W | | | | | | | | | | | | |
| 532 | Steric hindrance + | Q347R/K360E/D399V/ | 328 | 61 | 62 | 62.8 | 0.0 | 37.2 | 918 | 328 | 61 | 98.66 | 69 | 0.23 |
| | Charge | F405V/K409W | | | | | | | | | | | | |
| 533 | Steric hindrance + | Q347R/K360E/D399V/ | 329 | 61 | 62 | 62.3 | 0.2 | 37.4 | 919 | 329 | 61 | 97.95 | 67.8 | 0.25 |
| | Charge | F405S/K409W | | | | | | | | | | | | |
| 534 | Steric hindrance + | Q347R/K360E/D399V/ | 330 | 61 | 62 | 61.3 | 1.4 | 37.3 | 920 | 330 | 61 | 95.73 | 67.8 | 0.16 |
| | Charge + Disulfide | F405C/K409W | | | | | | | | | | | | |
| | bond | | | | | | | | | | | | | |
| 535 | Steric hindrance + | Q347R/K360E/D399V/ | 331 | 61 | 62 | 65.8 | 1.4 | 32.8 | 921 | 331 | 61 | 97.02 | 67.2 | 0.19 |
| | Charge | F405N/K409W | | | | | | | | | | | | |
| 536 | Steric hindrance + | Q347R/K360E/D399V/ | 332 | 61 | 62 | 61.2 | 2.2 | 36.5 | 922 | 332 | 61 | 96.39 | 65.4 | 0.2 |
| | Charge | F405D/K409W | | | | | | | | | | | | |
| 537 | Steric hindrance + | Q347R/K360E/D399V/ | 333 | 61 | 62 | 59.5 | 3.2 | 37.3 | 923 | 333 | 61 | 94.8 | 66 | 0.2 |
| | Charge | F405T/K409Y | | | | | | | | | | | | |
| 538 | Steric hindrance + | Q347R/K360E/D399V/ | 334 | 61 | 62 | 59.6 | 1.8 | 38.6 | 924 | 334 | 61 | 96.49 | 66 | 0.19 |
| | Charge | F405T/K409H | | | | | | | | | | | | |
| 539 | Steric hindrance + | Q347R/K360E/D399A/ | 335 | 61 | 62 | 62.8 | 0.5 | 36.7 | 925 | 335 | 61 | 97.81 | 67.8 | 0.21 |
| | Charge | F405T/K409F | | | | | | | | | | | | |
| 540 | Steric hindrance + | Q347R/K360E/D399L/ | 336 | 61 | 62 | 64.1 | 0.0 | 35.9 | 926 | 336 | 61 | 96.89 | 68.4 | 0.14 |
| | Charge | F405T/K409F | | | | | | | | | | | | |
| 541 | Steric hindrance + | Q347R/K360E/D399I/ | 337 | 61 | 62 | 64.4 | 0.0 | 35.6 | 927 | 337 | 61 | 97.37 | 68.4 | 0.15 |
| | Charge | F405T/K409F | | | | | | | | | | | | |
| 542 | Steric hindrance + | Q347R/K360E/D399M/ | 338 | 61 | 62 | 64.4 | 0.0 | 35.6 | 928 | 338 | 61 | 98.41 | 69 | 0.21 |
| | Charge | F405T/K409F | | | | | | | | | | | | |
| 543 | Steric hindrance + | Q347R/K360E/D399T/ | 339 | 61 | 62 | 64.2 | 0.0 | 35.8 | 929 | 339 | 61 | 98.03 | 69 | 0.19 |
| | Charge | F405T/K409F | | | | | | | | | | | | |
| 544 | Steric hindrance + | Q347R/K360E/D399C/ | 340 | 61 | 62 | 62.8 | 0.4 | 36.8 | 930 | 340 | 61 | 97.49 | 69 | 0.17 |
| | Charge + Disulfide | F405T/K409F | | | | | | | | | | | | |
| | bond | | | | | | | | | | | | | |
| 545 | Steric hindrance + | Q347R/K360E/D399N/ | 341 | 61 | 62 | 62.9 | 0.0 | 37.1 | 931 | 341 | 61 | 98.21 | 68.4 | 0.19 |
| | Charge | F405T/K409F | | | | | | | | | | | | |
| 546 | Steric hindrance + | Q347R/K360E/D399Q/ | 342 | 61 | 62 | 63.4 | 0.9 | 35.7 | 932 | 342 | 61 | 97.4 | 67.8 | 0.2 |
| | Charge | F405T/K409F | | | | | | | | | | | | |
| 547 | Steric hindrance + | Q347R/K360E/D399H/ | 343 | 61 | 62 | 67.3 | 0.4 | 32.3 | 933 | 343 | 61 | 96.36 | 67.2 | 0.16 |
| | Charge | F405T/K409F | | | | | | | | | | | | |
| 548 | Steric hindrance + | Q347R/K360E/D399A/ | 344 | 61 | 62 | 63.8 | 1.3 | 34.9 | 934 | 344 | 61 | 95.83 | 66.6 | 0.17 |
| | Charge | F405T/K409Y | | | | | | | | | | | | |
| 549 | Steric hindrance + | Q347R/K360E/D399L/ | 345 | 61 | 62 | 67.7 | 1.3 | 31.1 | 935 | 345 | 61 | 95.94 | 66 | 0.14 |
| | Charge | F405T/K409Y | | | | | | | | | | | | |
| 550 | Steric hindrance + | Q347R/K360E/D399M/ | 346 | 61 | 62 | 63.8 | 1.2 | 35.0 | 936 | 346 | 61 | 96.74 | 67.2 | 0.21 |
| | Charge | F405T/K409Y | | | | | | | | | | | | |
| 551 | Steric hindrance + | Q347R/K360E/D399S/ | 347 | 61 | 62 | 66.2 | 1.2 | 32.6 | 937 | 347 | 61 | 95.37 | 63.6 | 0.15 |
| | Charge | F405T/K409Y | | | | | | | | | | | | |
| 552 | Steric hindrance + | Q347R/K360E/D399T/ | 348 | 61 | 62 | 62.2 | 2.1 | 35.7 | 938 | 348 | 61 | 95.85 | 66.6 | 0.2 |
| | Charge | F405T/K409Y | | | | | | | | | | | | |
| 553 | Steric hindrance + | Q347R/K360E/D399C/ | 349 | 61 | 62 | 59.3 | 2.3 | 38.4 | 939 | 349 | 61 | 95.53 | 66.6 | 0.23 |
| | Charge + Disulfide | F405T/K409Y | | | | | | | | | | | | |
| | bond | | | | | | | | | | | | | |
| 554 | Steric hindrance + | Q347R/K360E/D399N/ | 350 | 61 | 62 | 64.3 | 0.3 | 35.4 | 940 | 350 | 61 | 97.11 | 68.4 | 0.16 |
| | Charge | F405T/K409Y | | | | | | | | | | | | |
| 555 | Steric hindrance + | Q347R/K360E/D399Q/ | 351 | 61 | 62 | 58.9 | 2.8 | 38.3 | 941 | 351 | 61 | 94.93 | 66.6 | 0.19 |
| | Charge | F405T/K409Y | | | | | | | | | | | | |
| 556 | Steric hindrance + | Q347R/K360E/D399H/ | 352 | 61 | 62 | 62.1 | 0.7 | 37.1 | 942 | 352 | 61 | 97.51 | 67.2 | 0.22 |
| | Charge | F405T/K409Y | | | | | | | | | | | | |
| 557 | Steric hindrance + | Q347R/K360E/D399V/ | 353 | 61 | 62 | 65.1 | 0.0 | 34.9 | 943 | 353 | 61 | 98.15 | 67.8 | 0.18 |
| | Charge | F405A/K409F | | | | | | | | | | | | |
| 558 | Steric hindrance + | Q347R/K360E/D399V/ | 354 | 61 | 62 | 66.7 | 0.0 | 33.3 | 944 | 354 | 61 | 97.13 | 68.4 | 0.15 |
| | Charge | F405V/K409F | | | | | | | | | | | | |
| 559 | Steric hindrance + | Q347R/K360E/D399V/ | 355 | 61 | 62 | 60.0 | 1.1 | 39.0 | 945 | 355 | 61 | 96.76 | 67.2 | 0.17 |
| | Charge | F405S/K409F | | | | | | | | | | | | |
| 560 | Steric hindrance + | Q347R/K360E/D399V/ | 356 | 61 | 62 | 62.4 | 2.0 | 35.6 | 946 | 356 | 61 | 95.59 | 67.8 | 0.16 |
| | Charge + Disulfide | F405C/K409F | | | | | | | | | | | | |
| | bond | | | | | | | | | | | | | |
| 561 | Steric hindrance + | Q347R/K360E/D399V/ | 357 | 61 | 62 | 64.9 | 2.7 | 32.4 | 947 | 357 | 61 | 94.5 | 62.4 | 0.15 |
| | Charge | F405D/K409F | | | | | | | | | | | | |
| 562 | Steric hindrance + | Q347R/K360E/D399V/ | 358 | 61 | 62 | 63.8 | 2.9 | 33.3 | 948 | 358 | 61 | 93.45 | 64.2 | 0.15 |
| | Charge | F405A/K409Y | | | | | | | | | | | | |
| 563 | Steric hindrance + | Q347R/K360E/D399V/ | 359 | 61 | 62 | 63.9 | 5.7 | 30.4 | 949 | 359 | 61 | 88.5 | 64.2 | 0.11 |
| | Charge + Disulfide | F405C/K409Y | | | | | | | | | | | | |
| | bond | | | | | | | | | | | | | |
| 564 | Steric hindrance + | Q347R/K360E/D399V/ | 360 | 61 | 62 | 53.0 | 8.8 | 38.3 | 950 | 360 | 61 | 84.57 | 60 | 0.2 |
| | Charge | F405N/K409Y | | | | | | | | | | | | |
| 565 | Steric hindrance + | Q347R/K360E/D399V/ | 361 | 61 | 62 | 57.0 | 3.2 | 39.8 | 951 | 361 | 61 | 94.89 | 63 | 0.23 |
| | Charge | F405D/K409Y | | | | | | | | | | | | |
| 566 | Steric hindrance + | Q347R/K360E/D399L/ | 362 | 61 | 62 | 65.6 | 0.0 | 34.4 | 952 | 362 | 61 | 98.49 | 68.4 | 0.21 |
| | Charge | F405A/K409W | | | | | | | | | | | | |
| 567 | Steric hindrance + | Q347R/K360E/D399L/ | 363 | 61 | 62 | 66.0 | 0.3 | 33.7 | 953 | 363 | 61 | 97.73 | 67.8 | 0.21 |
| | Charge | F405S/K409W | | | | | | | | | | | | |
| 568 | Steric hindrance + | Q347R/K360E/D399L/ | 364 | 61 | 62 | 64.3 | 1.7 | 34.0 | 954 | 364 | 61 | 95.13 | 67.8 | 0.19 |
| | Charge + Disulfide | F405C/K409W | | | | | | | | | | | | |
| | bond | | | | | | | | | | | | | |
| 569 | Steric hindrance + | Q347R/K360E/D399I/ | 365 | 61 | 62 | 63.8 | 0.3 | 35.9 | 955 | 365 | 61 | 98.21 | 52.8 | 0.21 |
| | Charge | F405S/K409W | | | | | | | | | | | | |
| 570 | Steric hindrance + | Q347R/K360E/D399I/ | 366 | 61 | 62 | 64.1 | 1.3 | 34.6 | 956 | 366 | 61 | 96.18 | 67.8 | 0.19 |
| | Charge + Disulfide | F405C/K409W | | | | | | | | | | | | |
| | bond | | | | | | | | | | | | | |
| 571 | Steric hindrance + | Q347R/K360E/D399M/ | 367 | 61 | 62 | 67.0 | 0.0 | 33.0 | 957 | 367 | 61 | 98.03 | 67.8 | 0.2 |
| | Charge | F405A/K409W | | | | | | | | | | | | |
| 572 | Steric hindrance + | Q347R/K360E/D399M/ | 368 | 61 | 62 | 63.6 | 0.4 | 36.1 | 958 | 368 | 61 | 97.92 | 67.8 | 0.2 |
| | Charge | F405S/K409W | | | | | | | | | | | | |
| 573 | Steric hindrance + | Q347R/K360E/D399M/ | 369 | 61 | 62 | 60.0 | 2.3 | 37.7 | 959 | 369 | 61 | 93.7 | 68.4 | 0.19 |
| | Charge + Disulfide | F405C/K409W | | | | | | | | | | | | |
| | bond | | | | | | | | | | | | | |
| 574 | Steric hindrance + | Q347R/K360E/D399G/ | 370 | 61 | 62 | 69.3 | 0.0 | 30.7 | 960 | 370 | 61 | 97.05 | 69 | 0.16 |
| | Charge | F405T/K409W | | | | | | | | | | | | |
| 575 | Steric hindrance + | Q347H/K360E/D399V/ | 371 | 61 | 62 | 66.1 | 0.0 | 33.9 | 961 | 371 | 61 | 98.13 | 68.4 | 0.2 |
| | Charge | F405T/K409W | | | | | | | | | | | | |
| 576 | Steric hindrance + | Q347R/K360E/D399A/ | 372 | 61 | 62 | 70.9 | 0.0 | 29.1 | 962 | 372 | 61 | 95.45 | 69 | 0.11 |
| | Charge | F405T/K409W | | | | | | | | | | | | |
| 577 | Steric hindrance + | Q347R/K360E/D399N/ | 373 | 61 | 62 | 67.1 | 0.0 | 32.9 | 963 | 373 | 61 | 97.1 | 69 | 0.16 |
| | Charge | F405T/K409W | | | | | | | | | | | | |
| 578 | Steric hindrance + | Q347R/K360E/D399Q/ | 374 | 61 | 62 | 73.5 | 0.7 | 25.8 | 964 | 374 | 61 | 93.84 | 68.4 | 0.07 |
| | Charge | F405T/K409W | | | | | | | | | | | | |
| 579 | Steric hindrance + | Q347R/K360E/D399S/ | 375 | 61 | 62 | 69.2 | 0.7 | 30.2 | 965 | 375 | 61 | 95.51 | 67.8 | 0.13 |
| | Charge | F405T/K409F | | | | | | | | | | | | |
| 580 | Steric hindrance + | Q347R/K360E/D399V/ | 376 | 61 | 62 | 72.0 | 1.3 | 26.7 | 966 | 376 | 61 | 94.02 | 66 | 0.09 |
| | Charge | F405N/K409F | | | | | | | | | | | | |
| 581 | Steric hindrance + | Q347R/K360E/D399V/ | 377 | 61 | 62 | 66.7 | 0.9 | 32.4 | 967 | 377 | 61 | 96.4 | 67.8 | 0.17 |
| | Charge | F405V/K409Y | | | | | | | | | | | | |
| 582 | Steric hindrance + | Q347R/K360E/D399V/ | 378 | 61 | 62 | 60.7 | 5.3 | 34.0 | 968 | 378 | 61 | 90.67 | 63.6 | 0.2 |
| | Charge | F405S/K409Y | | | | | | | | | | | | |
| 583 | Steric hindrance + | Q347R/K360E/D399I/ | 379 | 61 | 62 | 64.9 | 0.0 | 35.1 | 969 | 379 | 61 | 97.8 | 67.8 | 0.18 |
| | Charge | F405A/K409W | | | | | | | | | | | | |
| 584 | Steric hindrance + | Q347R/K360E/D399V/ | 380 | 61 | 62 | 63.2 | 0.9 | 35.9 | 970 | 380 | 61 | 97.48 | 66 | 0.18 |
| | Charge | F405G/K409W | | | | | | | | | | | | |
| 585 | Steric hindrance | T394W/F405S | 381 | 61 | 62 | 63.3 | 0.0 | 36.7 | 971 | 381 | 61 | 99.05 | 68.4 | 0.28 |
| 586 | Steric hindrance | T394W/F405T | 382 | 61 | 62 | 67.2 | 0.0 | 32.8 | 972 | 382 | 61 | 98.67 | 67.2 | 0.26 |
| 587 | Steric hindrance | T394Y/F405A | 383 | 61 | 62 | 58.3 | 3.2 | 38.5 | 973 | 383 | 61 | 99.12 | 64.8 | 0.3 |
| 588 | Steric hindrance | T394H/F405A | 384 | 61 | 62 | 34.0 | 39.4 | 26.6 | 974 | 384 | 61 | 98.59 | 63 | 0.25 |
| 589 | Steric hindrance | T394Y/F405T | 385 | 61 | 62 | 52.4 | 5.2 | 42.4 | 975 | 385 | 61 | 96.59 | 63 | 0.34 |
| 590 | Steric hindrance + | T394Y/F405C | 386 | 61 | 62 | 52.3 | 8.6 | 39.1 | 976 | 386 | 61 | 90.84 | 67.2 | 0.23 |
| | Disulfide bond | | | | | | | | | | | | | |
| 591 | Steric hindrance | T394Y/F405V | 387 | 61 | 62 | 42.8 | 16.3 | 40.9 | 977 | 387 | 61 | 73.47 | 61.2 | 0.3 |
| 592 | Steric hindrance | T394F/F405G | 388 | 61 | 62 | 57.9 | 5.6 | 36.5 | 978 | 388 | 61 | 99.01 | 63 | 0.26 |
| 593 | Steric hindrance + | T394F/F405C | 389 | 61 | 62 | 60.9 | 3.8 | 35.3 | 979 | 389 | 61 | 98.17 | 69 | 0.28 |
| | Disulfide bond | | | | | | | | | | | | | |
| 594 | Steric hindrance | T394F/F405V | 390 | 61 | 62 | 60.8 | 3.7 | 35.5 | 980 | 390 | 61 | 98.58 | 67.2 | 0.25 |
| 595 | Steric hindrance | T394H/F405G | 391 | 61 | 62 | 48.9 | 21.9 | 29.2 | 981 | 391 | 61 | 96.41 | 58.8 | 0.27 |
| 596 | Steric hindrance | T394H/F405S | 392 | 61 | 62 | 45.0 | 26.5 | 28.6 | 982 | 392 | 61 | 97.1 | 64.8 | 0.28 |
| 597 | Steric hindrance | T394H/F405T | 393 | 61 | 62 | 37.4 | 35.7 | 26.9 | 983 | 393 | 61 | 97.36 | 64.2 | 0.28 |
| 598 | Steric hindrance + | T394H/F405C | 394 | 61 | 62 | 20.2 | 59.9 | 19.8 | 984 | 394 | 61 | 94.34 | 67.2 | 0.23 |
| | Disulfide bond | | | | | | | | | | | | | |
| 599 | Disulfide bond | T394C/F405C | 395 | 61 | 62 | 9.0 | 67.1 | 23.9 | 985 | 395 | 61 | 92.35 | 67.8 | 0.27 |
| 600 | Steric hindrance + | T394W/F405C | 396 | 61 | 62 | 57.7 | 4.3 | 38.0 | 986 | 396 | 61 | 91.43 | 67.8 | 0.25 |
| | Disulfide bond | | | | | | | | | | | | | |
| 601 | Steric hindrance | T394W/F405V | 397 | 61 | 62 | 61.9 | 1.3 | 36.9 | 987 | 397 | 61 | 97.09 | 67.2 | 0.21 |
| 602 | Steric hindrance | T394Y/F405G | 398 | 61 | 62 | 61.0 | 2.3 | 36.7 | 988 | 398 | 61 | 95.65 | 62.4 | 0.21 |
| 603 | Steric hindrance | T394Y/F405S | 399 | 61 | 62 | 63.7 | 1.5 | 34.8 | 989 | 399 | 61 | 95.84 | 65.4 | 0.24 |
| 604 | Steric hindrance | T394F/F405S | 400 | 61 | 62 | 60.5 | 3.9 | 35.6 | 990 | 400 | 61 | 97.33 | 67.2 | 0.25 |
| 605 | Steric hindrance | T394F/F405T | 401 | 61 | 62 | 65.6 | 4.0 | 30.4 | 991 | 401 | 61 | 95.95 | 66.6 | 0.17 |
| 606 | Steric hindrance | T394H/F405V | 402 | 61 | 62 | 16.6 | 77.1 | 6.2 | 992 | 402 | 61 | 80.86 | 61.8 | 0.08 |
| 607 | Steric hindrance | T394W/F405G | 403 | 61 | 62 | 63.7 | 0.0 | 36.3 | 993 | 403 | 61 | 96.78 | 66.6 | 0.22 |
| 608 | Steric hindrance | T366W/L368S/Y407V | 404 | 61 | 62 | 56.9 | 4.0 | 39.2 | 994 | 404 | 61 | 98.62 | 67.2 | 0.28 |
| 609 | Steric hindrance | T366W/L368T/Y407V | 405 | 61 | 62 | 58.0 | 3.4 | 38.7 | 995 | 405 | 61 | 98.22 | 69 | 0.27 |
| 610 | Steric hindrance + | T366W/L368C/Y407V | 406 | 61 | 62 | 60.9 | 0.0 | 39.1 | 996 | 406 | 61 | 96.88 | 69 | 0.31 |
| | Disulfide bond | | | | | | | | | | | | | |
| 611 | Steric hindrance | T366W/L368V/Y407V | 407 | 61 | 62 | 59.3 | 4.5 | 36.2 | 997 | 407 | 61 | 98.55 | 69 | 0.28 |
| 612 | Steric hindrance | T366W/L368G/Y407V | 408 | 61 | 62 | 63.6 | 0.0 | 36.4 | 998 | 408 | 61 | 98.78 | 67.2 | 0.29 |
| 613 | Steric hindrance | T366Y/L368A/Y407V | 409 | 61 | 62 | 61.2 | 0.0 | 38.8 | 999 | 409 | 61 | 98.59 | 67.8 | 0.26 |
| 614 | Steric hindrance | T366F/L368A/Y407V | 410 | 61 | 62 | 61.4 | 0.0 | 38.6 | 1000 | 410 | 61 | 98.73 | 67.2 | 0.28 |
| 615 | Steric hindrance | T366H/L368A/Y407V | 411 | 61 | 62 | 20.5 | 60.9 | 18.6 | 1001 | 411 | 61 | 96.33 | 64.2 | 0.26 |
| 616 | Steric hindrance | T366W/L368A/Y407L | 412 | 61 | 62 | 65.5 | 0.0 | 34.5 | 1002 | 412 | 61 | 97.67 | 66.6 | 0.21 |
| 617 | Steric hindrance | T366W/L368A/Y407I | 413 | 61 | 62 | 62.4 | 3.1 | 34.5 | 1003 | 413 | 61 | 98.11 | 68.4 | 0.2 |
| 618 | Steric hindrance | T366W/L368A/Y407M | 414 | 61 | 62 | 61.3 | 1.9 | 36.8 | 1004 | 414 | 61 | 98.02 | 67.8 | 0.21 |
| 619 | Steric hindrance | T366W/L368A/Y407A | 415 | 61 | 62 | 60.6 | 2.6 | 36.7 | 1005 | 415 | 61 | 98.98 | 67.8 | 0.01 |
| 620 | Steric hindrance | T366W/L368A/Y407S | 416 | 61 | 62 | 62.6 | 1.2 | 36.2 | 1006 | 416 | 61 | 96.53 | 66 | 0.17 |
| 621 | Steric hindrance | T366W/L368A/Y407T | 417 | 61 | 62 | 65.9 | 0.4 | 33.8 | 1007 | 417 | 61 | 97.14 | 67.2 | 0.22 |
| 622 | Steric hindrance + | T366W/L368A/Y407C | 418 | 61 | 62 | 60.5 | 4.4 | 35.1 | 1008 | 418 | 61 | 95.38 | 68.4 | 0.21 |
| | Disulfide bond | | | | | | | | | | | | | |
| 623 | Steric hindrance | T366W/L368A/Y407H | 419 | 61 | 62 | 28.0 | 35.5 | 36.5 | 1009 | 419 | 61 | 57.08 | 54.6 | 0.21 |
| 624 | Steric hindrance | T366W/L368A/Y407N | 420 | 61 | 62 | 58.5 | 3.4 | 38.1 | 1010 | 420 | 61 | 93.71 | 60 | 0.22 |
| 625 | Steric hindrance + | T366W/L368C/Y407C | 421 | 61 | 62 | 61.8 | 0.0 | 38.2 | 1011 | 421 | 61 | 97.93 | 67.8 | 0.21 |
| | Disulfide bond | | | | | | | | | | | | | |
| 626 | Steric hindrance + | T366C/L368A/Y407C | 422 | 61 | 62 | 5.1 | 63.8 | 31.0 | 1012 | 422 | 61 | 56.77 | 60.6 | 0.1 |
| | Disulfide bond | | | | | | | | | | | | | |
| 627 | Steric hindrance | T366W/L368S/Y407L | 423 | 61 | 62 | 56.7 | 5.2 | 38.1 | 1013 | 423 | 61 | 94.19 | 63 | 0.22 |
| 628 | Steric hindrance | T366W/L368T/Y407L | 424 | 61 | 62 | 63.1 | 2.8 | 34.1 | 1014 | 424 | 61 | 97.73 | 66 | 0.17 |
| 629 | Steric hindrance + | T366W/L368C/Y407L | 425 | 61 | 62 | 62.4 | 2.1 | 35.5 | 1015 | 425 | 61 | 94.95 | 66 | 0.22 |
| | Disulfide bond | | | | | | | | | | | | | |
| 630 | Steric hindrance | T366W/L368V/Y407L | 426 | 61 | 62 | 62.1 | 2.5 | 35.4 | 1016 | 426 | 61 | 98.65 | 67.2 | 0.23 |
| 631 | Steric hindrance | T366W/L368G/Y407L | 427 | 61 | 62 | 60.3 | 2.1 | 37.6 | 1017 | 427 | 61 | 95.76 | 72 | 0.18 |
| 632 | Steric hindrance | T366W/L368S/Y407I | 428 | 61 | 62 | 53.5 | 10.9 | 35.6 | 1018 | 428 | 61 | 96.32 | 66 | 0.22 |
| 633 | Steric hindrance + | T366W/L368C/Y407I | 429 | 61 | 62 | 59.1 | 3.4 | 37.5 | 1019 | 429 | 61 | 95.18 | 67.8 | 0.22 |
| | Disulfide bond | | | | | | | | | | | | | |
| 634 | Steric hindrance | T366W/L368V/Y407I | 430 | 61 | 62 | 59.7 | 4.5 | 35.8 | 1020 | 430 | 61 | 98.66 | 67.8 | 0.22 |
| 635 | Steric hindrance | T366W/L368G/Y407I | 431 | 61 | 62 | 58.2 | 6.1 | 35.7 | 1021 | 431 | 61 | 96.76 | 64.8 | 0.22 |
| 636 | Steric hindrance | T366W/L368S/Y407M | 432 | 61 | 62 | 54.6 | 5.6 | 39.8 | 1022 | 432 | 61 | 96.55 | 65.4 | 0.22 |
| 637 | Steric hindrance | T366W/L368T/Y407M | 433 | 61 | 62 | 60.6 | 3.0 | 36.4 | 1023 | 433 | 61 | 97.12 | 65.4 | 0.23 |
| 638 | Steric hindrance + | T366W/L368C/Y407M | 434 | 61 | 62 | 60.5 | 4.4 | 35.2 | 1024 | 434 | 61 | 94.73 | 66 | 0.2 |
| | Disulfide bond | | | | | | | | | | | | | |
| 639 | Steric hindrance | T366W/L368V/Y407M | 435 | 61 | 62 | 56.7 | 3.5 | 39.8 | 1025 | 435 | 61 | 96.02 | 66.6 | 0.2 |
| 640 | Steric hindrance | T366W/L368G/Y407M | 436 | 61 | 62 | 55.7 | 5.6 | 38.7 | 1026 | 436 | 61 | 97.25 | 64.8 | 0.25 |
| 641 | Steric hindrance | T366W/L368S/Y407A | 437 | 61 | 62 | 57.6 | 3.5 | 38.9 | 1027 | 437 | 61 | 97.38 | 66 | 0.22 |
| 642 | Steric hindrance | T366W/L368T/Y407A | 438 | 61 | 62 | 57.3 | 4.3 | 38.4 | 1028 | 438 | 61 | 98.42 | 68.4 | 0.22 |
| 643 | Steric hindrance + | T366W/L368C/Y407A | 439 | 61 | 62 | 49.4 | 4.1 | 46.5 | 1029 | 439 | 61 | 94.57 | 69 | 0.22 |
| | Disulfide bond | | | | | | | | | | | | | |
| 644 | Steric hindrance | T366W/L368V/Y407A | 440 | 61 | 62 | 56.9 | 5.1 | 37.9 | 1030 | 440 | 61 | 98.72 | 68.4 | 0.2 |
| 645 | Steric hindrance | T366W/L368G/Y407A | 441 | 61 | 62 | 61.6 | 0.0 | 38.4 | 1031 | 441 | 61 | 98.12 | 66 | 0.24 |
| 646 | Steric hindrance | T366W/L368S/Y407T | 442 | 61 | 62 | 63.6 | 1.2 | 35.2 | 1032 | 442 | 61 | 95.99 | 64.8 | 0.23 |
| 647 | Steric hindrance | T366W/L368T/Y407T | 443 | 61 | 62 | 63.7 | 0.6 | 35.7 | 1033 | 443 | 61 | 96.69 | 66.6 | 0.21 |
| 648 | Steric hindrance + | T366W/L368C/Y407T | 444 | 61 | 62 | 55.0 | 1.3 | 43.7 | 1034 | 444 | 61 | 94.61 | 68.4 | 0.25 |
| | Disulfide bond | | | | | | | | | | | | | |
| 649 | Steric hindrance | T366W/L368V/Y407T | 445 | 61 | 62 | 62.7 | 3.7 | 33.6 | 1035 | 445 | 61 | 96.19 | 67.8 | 0.23 |
| 650 | Steric hindrance | T366W/L368G/Y407T | 446 | 61 | 62 | 58.9 | 1.5 | 39.6 | 1036 | 446 | 61 | 95.89 | 63.6 | 0.24 |
| 651 | Steric hindrance + | T366W/L368S/Y407C | 447 | 61 | 62 | 46.8 | 4.7 | 48.5 | 1037 | 447 | 61 | 96.66 | 66.6 | 0.2 |
| | Disulfide bond | | | | | | | | | | | | | |
| 652 | Steric hindrance + | T366W/L368T/Y407C | 448 | 61 | 62 | 56.8 | 6.6 | 36.6 | 1038 | 448 | 61 | 95.78 | 68.4 | 0.19 |
| | Disulfide bond | | | | | | | | | | | | | |
| 653 | Steric hindrance + | T366W/L368V/Y407C | 449 | 61 | 62 | 55.0 | 8.9 | 36.1 | 1039 | 449 | 61 | 91.35 | 68.4 | 0.21 |
| | Disulfide bond | | | | | | | | | | | | | |
| 654 | Steric hindrance + | T366W/L368G/Y407C | 450 | 61 | 62 | 60.1 | 0.6 | 39.3 | 1040 | 450 | 61 | 96.64 | 66 | 0.21 |
| | Disulfide bond | | | | | | | | | | | | | |
| 655 | Steric hindrance | T366W/L368S/Y407H | 451 | 61 | 62 | 25.3 | 28.4 | 46.3 | 1041 | 451 | 61 | 53.32 | 53.4 | 0.25 |
| 656 | Steric hindrance | T366W/L368T/Y407H | 452 | 61 | 62 | 39.0 | 20.6 | 40.4 | 1042 | 452 | 61 | 59.36 | 55.2 | 0.24 |
| 657 | Steric hindrance | T366W/L368V/Y407H | 453 | 61 | 62 | 29.4 | 38.4 | 32.2 | 1043 | 453 | 61 | 58.63 | 58.2 | 0.23 |
| 658 | Steric hindrance | T366W/L368G/Y407H | 454 | 61 | 62 | 26.5 | 11.9 | 61.6 | 1044 | 454 | 61 | 66.71 | 53.4 | 0.19 |
| 659 | Steric hindrance | T366W/L368S/Y407N | 455 | 61 | 62 | 49.0 | 6.9 | 44.1 | 1045 | 455 | 61 | 85.83 | 54.6 | 0.22 |
| 660 | Steric hindrance | T366W/L368T/Y407N | 456 | 61 | 62 | 62.7 | 3.7 | 33.6 | 1046 | 456 | 61 | 91.72 | 58.2 | 0.19 |
| 661 | Steric hindrance + | T366W/L368C/Y407N | 457 | 61 | 62 | 49.2 | 11.0 | 39.8 | 1047 | 457 | 61 | 78.42 | 55.2 | 0.19 |
| | Disulfide bond | | | | | | | | | | | | | |
| 662 | Steric hindrance | T366W/L368G/Y407N | 458 | 61 | 62 | 31.3 | 10.1 | 58.6 | 1048 | 458 | 61 | 69.65 | 52.2 | 0.18 |
| 663 | Steric hindrance | T366W/L368S/Y407Q | 459 | 61 | 62 | 48.7 | 6.0 | 45.3 | 1049 | 459 | 61 | 89.65 | 56.4 | 0.22 |
| 664 | Steric hindrance | T366W/L368T/Y407Q | 460 | 61 | 62 | 49.3 | 8.6 | 42.2 | 1050 | 460 | 61 | 75.82 | 57 | 0.08 |
| 665 | Steric hindrance + | T366W/L368C/Y407Q | 461 | 61 | 62 | 42.5 | 16.5 | 41.0 | 1051 | 461 | 61 | 62.48 | 56.4 | 0.16 |
| | Disulfide bond | | | | | | | | | | | | | |
| 666 | Steric hindrance | T366W/L368V/Y407Q | 462 | 61 | 62 | 28.6 | 35.9 | 35.5 | 1052 | 462 | 61 | 62.16 | 57.6 | 0.25 |
| 667 | Steric hindrance | T366W/L368G/Y407Q | 463 | 61 | 62 | 24.9 | 28.3 | 46.8 | 1053 | 463 | 61 | 62.44 | 53.4 | 0.09 |
| 668 | Steric hindrance | T366Y/L368S/Y407L | 464 | 61 | 62 | 57.4 | 9.0 | 33.6 | 1054 | 464 | 61 | 96.1 | 66.6 | 0.19 |
| 669 | Steric hindrance + | T366Y/L368C/Y407L | 465 | 61 | 62 | 31.0 | 6.9 | 62.0 | 1055 | 465 | 61 | 95.43 | 67.2 | 0.23 |
| | Disulfide bond | | | | | | | | | | | | | |
| 670 | Steric hindrance | T366Y/L368V/Y407L | 466 | 61 | 62 | 57.3 | 3.1 | 39.6 | 1056 | 466 | 61 | 97.98 | 69 | 0.2 |
| 671 | Steric hindrance | T366Y/L368G/Y407L | 467 | 61 | 62 | 52.1 | 11.1 | 36.8 | 1057 | 467 | 61 | 93.15 | 75.6 | 0.16 |
| 672 | Steric hindrance | T366Y/L368S/Y407I | 468 | 61 | 62 | 58.4 | 3.4 | 38.2 | 1058 | 468 | 61 | 98.75 | 67.8 | 0.01 |
| 673 | Steric hindrance + | T366Y/L368C/Y407I | 469 | 61 | 62 | 65.2 | 1.5 | 33.3 | 1059 | 469 | 61 | 94.16 | 67.8 | 0.16 |
| | Disulfide bond | | | | | | | | | | | | | |
| 674 | Steric hindrance | T366Y/L368V/Y407I | 470 | 61 | 62 | 61.9 | 2.4 | 35.7 | 1060 | 470 | 61 | 97.68 | 68.4 | 0.19 |
| 675 | Steric hindrance | T366Y/L368G/Y407I | 471 | 61 | 62 | 51.1 | 7.3 | 41.6 | 1061 | 471 | 61 | 94.77 | 62.4 | 0.19 |
| 676 | Steric hindrance | T366Y/L368G/Y407M | 472 | 61 | 62 | 57.2 | 8.3 | 34.5 | 1062 | 472 | 61 | 90.25 | 61.2 | 0.04 |
| 677 | Steric hindrance | T366Y/L368V/Y407A | 473 | 61 | 62 | 66.5 | 0.0 | 33.5 | 1063 | 473 | 61 | 97.7 | 67.8 | 0.21 |
| 678 | Steric hindrance | T366Y/L368G/Y407A | 474 | 61 | 62 | 43.6 | 16.2 | 40.2 | 1064 | 474 | 61 | 92.18 | 66.6 | 0.2 |
| 679 | Steric hindrance | T366Y/L368S/Y407T | 475 | 61 | 62 | 58.3 | 3.2 | 38.5 | 1065 | 475 | 61 | 93.85 | 63 | 0.23 |
| 680 | Steric hindrance | T366Y/L368T/Y407T | 476 | 61 | 62 | 59.2 | 3.9 | 36.9 | 1066 | 476 | 61 | 92.9 | 63 | 0.2 |
| 681 | Steric hindrance + | T366Y/L368C/Y407T | 477 | 61 | 62 | 54.6 | 3.9 | 41.5 | 1067 | 477 | 61 | 92.82 | 64.8 | 0.22 |
| | Disulfide bond | | | | | | | | | | | | | |
| 682 | Steric hindrance | T366Y/L368V/Y407T | 478 | 61 | 62 | 63.6 | 1.9 | 34.6 | 1068 | 478 | 61 | 95.64 | 67.2 | 0.23 |
| 683 | Steric hindrance | T366Y/L368G/Y407T | 479 | 61 | 62 | 45.2 | 11.1 | 43.7 | 1069 | 479 | 61 | 78.6 | 55.8 | 0.21 |
| 684 | Steric hindrance + | T366Y/L368T/Y407C | 480 | 61 | 62 | 54.1 | 6.9 | 39.0 | 1070 | 480 | 61 | 96.32 | 66 | 0.22 |
| | Disulfide bond | | | | | | | | | | | | | |
| 685 | Steric hindrance + | T366Y/L368V/Y407C | 481 | 61 | 62 | 58.3 | 2.5 | 39.2 | 1071 | 481 | 61 | 93.21 | 68.4 | 0.24 |
| | Disulfide bond | | | | | | | | | | | | | |
| 686 | Steric hindrance + | T366Y/L368G/Y407C | 482 | 61 | 62 | 44.1 | 11.5 | 44.3 | 1072 | 482 | 61 | 90.88 | 63.6 | 0.19 |
| | Disulfide bond | | | | | | | | | | | | | |
| 687 | Steric hindrance | T366Y/L368S/Y407H | 483 | 61 | 62 | 45.6 | 11.6 | 42.9 | 1073 | 483 | 61 | 74.46 | 55.2 | 0.22 |
| 688 | Steric hindrance | T366Y/L368T/Y407H | 484 | 61 | 62 | 47.1 | 11.3 | 41.6 | 1074 | 484 | 61 | 79.63 | 57 | 0.23 |
| 689 | Steric hindrance + | T366Y/L368C/Y407H | 485 | 61 | 62 | 42.6 | 13.5 | 44.0 | 1075 | 485 | 61 | 72 | 57 | 0.24 |
| | Disulfide bond | | | | | | | | | | | | | |
| 690 | Steric hindrance | T366Y/L368V/Y407H | 486 | 61 | 62 | 40.3 | 23.6 | 36.1 | 1076 | 486 | 61 | 57.82 | 60.6 | 0.27 |
| 691 | Steric hindrance | T366Y/L368G/Y407H | 487 | 61 | 62 | 19.8 | 28.6 | 51.7 | 1077 | 487 | 61 | 49.01 | 51.6 | 0.14 |
| 692 | Steric hindrance | T366Y/L368S/Y407N | 488 | 61 | 62 | 50.4 | 7.4 | 42.2 | 1078 | 488 | 61 | 86.8 | 54.6 | 0.22 |
| 693 | Steric hindrance | T366Y/L368T/Y407N | 489 | 61 | 62 | 45.3 | 11.0 | 43.7 | 1079 | 489 | 61 | 79.68 | 54 | 0.23 |
| 694 | Steric hindrance + | T366Y/L368C/Y407N | 490 | 61 | 62 | 43.3 | 13.1 | 43.6 | 1080 | 490 | 61 | 74.89 | 54 | 0.24 |
| | Disulfide bond | | | | | | | | | | | | | |
| 695 | Steric hindrance | T366Y/L368V/Y407N | 491 | 61 | 62 | 51.9 | 12.3 | 35.8 | 1081 | 491 | 61 | 76.44 | 61.2 | 0.25 |
| 696 | Steric hindrance | T366Y/L368G/Y407N | 492 | 61 | 62 | 16.1 | 20.3 | 63.5 | 1082 | 492 | 61 | 56.22 | 94.2 | 0.14 |
| 697 | Steric hindrance | T366Y/L368S/Y407Q | 493 | 61 | 62 | 46.5 | 8.9 | 44.5 | 1083 | 493 | 61 | 80.63 | 55.8 | 0.2 |
| 698 | Steric hindrance | T366Y/L368T/Y407Q | 494 | 61 | 62 | 40.3 | 15.3 | 44.4 | 1084 | 494 | 61 | 65.43 | 55.2 | 0.2 |
| 699 | Steric hindrance + | T366Y/L368C/Y407Q | 495 | 61 | 62 | 43.6 | 13.7 | 42.7 | 1085 | 495 | 61 | 78.31 | 56.4 | 0.19 |
| | Disulfide bond | | | | | | | | | | | | | |
| 700 | Steric hindrance | T366W/L368A/Y407Q | 496 | 61 | 62 | 55.3 | 3.4 | 41.2 | 1086 | 496 | 61 | 93.08 | 60 | 0.23 |
| 701 | Steric hindrance | T366W/L368T/Y407I | 497 | 61 | 62 | 64.2 | 0.0 | 35.8 | 1087 | 497 | 61 | 98.13 | 67.2 | 0.24 |
| 702 | Steric hindrance + | T366W/L368C/Y407H | 498 | 61 | 62 | 32.0 | 26.5 | 41.5 | 1088 | 498 | 61 | 52.44 | 57.6 | 0.25 |
| | Disulfide bond | | | | | | | | | | | | | |
| 703 | Steric hindrance | T366W/L368V/Y407N | 499 | 61 | 62 | 33.7 | 29.7 | 36.6 | 1089 | 499 | 61 | 51.46 | 55.2 | 0.26 |
| 704 | Steric hindrance | T366Y/L368T/Y407L | 500 | 61 | 62 | 50.0 | 15.4 | 34.6 | 1090 | 500 | 61 | 97.09 | 66 | 0.24 |
| 705 | Steric hindrance + | T366Y/L368C/Y407C | 501 | 61 | 62 | 57.0 | 1.3 | 41.7 | 1091 | 501 | 61 | 96.7 | 64.8 | 0.17 |
| | Disulfide bond | | | | | | | | | | | | | |
| 706 | Steric hindrance | T366F/L368S/Y407L | 502 | 61 | 62 | 55.4 | 7.7 | 36.8 | 1092 | 502 | 61 | 96.07 | 64.2 | 0.23 |
| 707 | Steric hindrance | T366F/L368T/Y407L | 503 | 61 | 62 | 52.7 | 11.4 | 35.8 | 1093 | 503 | 61 | 97.05 | 65.4 | 0.22 |
| 708 | Steric hindrance + | T366F/L368C/Y407L | 504 | 61 | 62 | 59.1 | 4.0 | 36.9 | 1094 | 504 | 61 | 95.62 | 66.6 | 0.22 |
| | Disulfide bond | | | | | | | | | | | | | |
| 709 | Steric hindrance | T366F/L368V/Y407L | 505 | 61 | 62 | 62.1 | 0.0 | 37.9 | 1095 | 505 | 61 | 98.14 | 68.4 | 0.23 |
| 710 | Steric hindrance | T366F/L368G/Y407L | 506 | 61 | 62 | 55.4 | 6.0 | 38.6 | 1096 | 506 | 61 | 93.82 | 78 | 0.19 |
| 711 | Steric hindrance | T366F/L368S/Y407I | 507 | 61 | 62 | 60.4 | 5.3 | 34.3 | 1097 | 507 | 61 | 96.31 | 66 | 0.19 |
| 712 | Steric hindrance | T366F/L368T/Y407I | 508 | 61 | 62 | 55.1 | 5.3 | 39.7 | 1098 | 508 | 61 | 96.55 | 65.4 | 0.23 |
| 713 | Steric hindrance + | T366F/L368C/Y407I | 509 | 61 | 62 | 60.2 | 1.8 | 38.0 | 1099 | 509 | 61 | 95.42 | 67.2 | 0.24 |
| | Disulfide bond | | | | | | | | | | | | | |
| 714 | Steric hindrance | T366F/L368V/Y407I | 510 | 61 | 62 | 61.9 | 0.0 | 38.1 | 1100 | 510 | 61 | 93.8 | 67.8 | 0.22 |
| 715 | Steric hindrance | T366F/L368G/Y407I | 511 | 61 | 62 | 54.4 | 8.9 | 36.8 | 1101 | 511 | 61 | 88.22 | 60.6 | 0.19 |
| 716 | Steric hindrance | T366F/L368S/Y407M | 512 | 61 | 62 | 55.2 | 5.8 | 39.0 | 1102 | 512 | 61 | 96.03 | 64.2 | 0.23 |
| 717 | Steric hindrance | T366F/L368T/Y407M | 513 | 61 | 62 | 52.3 | 9.9 | 37.8 | 1103 | 513 | 61 | 96.16 | 64.8 | 0.21 |
| 718 | Steric hindrance + | T366F/L368C/Y407M | 514 | 61 | 62 | 52.4 | 4.0 | 43.5 | 1104 | 514 | 61 | 94.83 | 66.6 | 0.23 |
| | Disulfide bond | | | | | | | | | | | | | |
| 719 | Steric hindrance | T366F/L368V/Y407M | 515 | 61 | 62 | 62.1 | 0.0 | 37.9 | 1105 | 515 | 61 | 98.04 | 68.4 | 0.24 |
| 720 | Steric hindrance | T366F/L368G/Y407M | 516 | 61 | 62 | 55.5 | 11.8 | 32.7 | 1106 | 516 | 61 | 91.42 | 59.4 | 0.16 |
| 721 | Steric hindrance | T366F/L368S/Y407A | 517 | 61 | 62 | 57.2 | 5.9 | 36.9 | 1107 | 517 | 61 | 96.82 | 64.2 | 0.22 |
| 722 | Steric hindrance | T366F/L368T/Y407A | 518 | 61 | 62 | 56.8 | 7.4 | 35.8 | 1108 | 518 | 61 | 97.21 | 64.8 | 0.2 |
| 723 | Steric hindrance + | T366F/L368C/Y407A | 519 | 61 | 62 | 59.2 | 4.2 | 36.5 | 1109 | 519 | 61 | 94.78 | 66.6 | 0.17 |
| | Disulfide bond | | | | | | | | | | | | | |
| 724 | Steric hindrance | T366F/L368V/Y407A | 520 | 61 | 62 | 63.7 | 2.6 | 33.7 | 1110 | 520 | 61 | 95.93 | 67.8 | 0.14 |
| 725 | Steric hindrance | T366F/L368G/Y407A | 521 | 61 | 62 | 44.7 | 18.9 | 36.5 | 1111 | 521 | 61 | 93 | 57.6 | 0.2 |
| 726 | Steric hindrance | T366F/L368S/Y407T | 522 | 61 | 62 | 11.6 | 2.7 | 85.7 | 1112 | 522 | 61 | 91.24 | 60.6 | 0.16 |
| 727 | Steric hindrance | T366F/L368T/Y407T | 523 | 61 | 62 | 61.8 | 2.2 | 36.0 | 1113 | 523 | 61 | 94.78 | 64.2 | 0.17 |
| 728 | Steric hindrance + | T366F/L368C/Y407T | 524 | 61 | 62 | 59.2 | 4.1 | 36.7 | 1114 | 524 | 61 | 91.42 | 64.2 | 0.12 |
| | Disulfide bond | | | | | | | | | | | | | |
| 729 | Steric hindrance | T366F/L368V/Y407T | 525 | 61 | 62 | 58.1 | 1.9 | 40.0 | 1115 | 525 | 61 | 96.13 | 67.2 | 0.2 |
| 730 | Steric hindrance | T366F/L368G/Y407T | 526 | 61 | 62 | 42.4 | 14.7 | 42.9 | 1116 | 526 | 61 | 68.82 | 54.6 | 0.16 |
| 731 | Steric hindrance + | T366F/L368S/Y407C | 527 | 61 | 62 | 47.9 | 5.2 | 46.9 | 1117 | 527 | 61 | 95.97 | 64.2 | 0.17 |
| | Disulfide bond | | | | | | | | | | | | | |
| 732 | Steric hindrance + | T366F/L368T/Y407C | 528 | 61 | 62 | 62.9 | 5.2 | 31.8 | 1118 | 528 | 61 | 95.49 | 65.4 | 0.12 |
| | Disulfide bond | | | | | | | | | | | | | |
| 733 | Steric hindrance + | T366F/L368V/Y407C | 529 | 61 | 62 | 61.0 | 2.1 | 36.9 | 1119 | 529 | 61 | 93.2 | 68.4 | 0.18 |
| | Disulfide bond | | | | | | | | | | | | | |
| 734 | Steric hindrance + | T366F/L368G/Y407C | 530 | 61 | 62 | 37.9 | 21.6 | 40.5 | 1120 | 530 | 61 | 90.66 | 64.8 | 0.16 |
| | Disulfide bond | | | | | | | | | | | | | |
| 735 | Steric hindrance | T366F/L368S/Y407H | 531 | 61 | 62 | 30.2 | 18.4 | 51.4 | 1121 | 531 | 61 | 59.38 | 52.8 | 0.19 |
| 736 | Steric hindrance | T366F/L368T/Y407H | 532 | 61 | 62 | 35.7 | 15.9 | 48.4 | 1122 | 532 | 61 | 70.92 | 54 | 0.22 |
| 737 | Steric hindrance + | T366F/L368C/Y407H | 533 | 61 | 62 | 43.8 | 14.3 | 41.9 | 1123 | 533 | 61 | 73.11 | 65.4 | 0.19 |
| | Disulfide bond | | | | | | | | | | | | | |
| 738 | Steric hindrance | T366F/L368G/Y407H | 534 | 61 | 62 | 25.9 | 26.2 | 47.8 | 1124 | 534 | 61 | 50.76 | 52.2 | 0.19 |
| 739 | Steric hindrance | T366F/L368S/Y407N | 535 | 61 | 62 | 27.5 | 15.1 | 57.4 | 1125 | 535 | 61 | 61.65 | 51 | 0.2 |
| 740 | Steric hindrance + | T366F/L368C/Y407N | 536 | 61 | 62 | 39.4 | 12.9 | 47.7 | 1126 | 536 | 61 | 72.42 | 53.4 | 0.22 |
| | Disulfide bond | | | | | | | | | | | | | |
| 741 | Steric hindrance | T366F/L368V/Y407N | 537 | 61 | 62 | 48.6 | 11.2 | 40.2 | 1127 | 537 | 61 | 83.11 | 60 | 0.22 |
| 742 | Steric hindrance | T366F/L368G/Y407N | 538 | 61 | 62 | 11.5 | 20.9 | 67.6 | 1128 | 538 | 61 | 61.64 | 50.4 | 0.16 |
| 743 | Steric hindrance | T366F/L368S/Y407Q | 539 | 61 | 62 | 29.0 | 11.8 | 59.2 | 1129 | 539 | 61 | 65 | 52.8 | 0.19 |
| 744 | Steric hindrance | T366F/L368T/Y407Q | 540 | 61 | 62 | 36.2 | 14.6 | 49.2 | 1130 | 540 | 61 | 68.88 | 55.2 | 0.2 |
| 745 | Steric hindrance + | T366F/L368C/Y407Q | 541 | 61 | 62 | 36.4 | 21.5 | 42.1 | 1131 | 541 | 61 | 58.87 | 55.8 | 0.19 |
| | Disulfide bond | | | | | | | | | | | | | |
| 746 | Steric hindrance | T366F/L368V/Y407Q | 542 | 61 | 62 | 31.6 | 28.1 | 40.2 | 1132 | 542 | 61 | 53.09 | 57 | 0.23 |
| 747 | Steric hindrance | T366F/L368G/Y407Q | 543 | 61 | 62 | 16.4 | 20.9 | 62.6 | 1133 | 543 | 61 | 47.58 | 51.6 | 0.15 |
| 748 | Steric hindrance + | T366F/L368C/Y407C | 544 | 61 | 62 | 58.0 | 0.0 | 42.0 | 1134 | 544 | 61 | 97.71 | 66 | 0.19 |
| | Disulfide bond | | | | | | | | | | | | | |
| 749 | Steric hindrance | T366Y/L368T/Y407I | 545 | 61 | 62 | 59.6 | 4.2 | 36.1 | 1135 | 545 | 61 | 96.94 | 66.6 | 0.2 |
| 750 | Steric hindrance | T366F/L368V/Y407H | 546 | 61 | 62 | 50.5 | 21.4 | 28.1 | 1136 | 546 | 61 | 63.31 | 58.2 | 0.13 |
| 751 | Steric hindrance | T366F/L368T/Y407N | 547 | 61 | 62 | 36.6 | 12.9 | 50.5 | 1137 | 547 | 61 | 68.95 | 51.6 | 0.16 |
| 752 | Steric hindrance + | T366Y/L368S/Y407C | 548 | 61 | 62 | 59.9 | 1.3 | 38.8 | 1138 | 548 | 61 | 96.16 | 66 | 0.21 |
| | Disulfide bond | | | | | | | | | | | | | |
| 753 | Steric hindrance | T366Y/L368V/Y407Q | 549 | 61 | 62 | 40.2 | 21.7 | 38.2 | 1139 | 549 | 61 | 61.87 | 59.4 | 0.26 |
| 754 | Steric hindrance | T366Y/L368S/Y407M | 550 | 61 | 62 | 57.9 | 4.5 | 37.6 | 1140 | 550 | 61 | 96.63 | 67.2 | 0.2 |
| 755 | Steric hindrance | T366Y/L368T/Y407M | 551 | 61 | 62 | 55.6 | 8.8 | 35.6 | 1141 | 551 | 61 | 96.86 | 66.6 | 0.2 |
| 756 | Steric hindrance + | T366Y/L368C/Y407M | 552 | 61 | 62 | 55.7 | 5.3 | 39.0 | 1142 | 552 | 61 | 95.11 | 67.2 | 0.19 |
| | Disulfide bond | | | | | | | | | | | | | |
| 757 | Steric hindrance | T366Y/L368V/Y407M | 553 | 61 | 62 | 66.1 | 0.0 | 33.9 | 1143 | 553 | 61 | 98.67 | 69 | 0.23 |
| 758 | Steric hindrance | T366Y/L368S/Y407A | 554 | 61 | 62 | 65.0 | 1.1 | 33.9 | 1144 | 554 | 61 | 96.86 | 64.8 | 0.21 |
| 759 | Steric hindrance | T366Y/L368T/Y407A | 555 | 61 | 62 | 61.3 | 3.6 | 35.1 | 1145 | 555 | 61 | 97.83 | 66 | 0.2 |
| 760 | Steric hindrance + | T366Y/L368C/Y407A | 556 | 61 | 62 | 57.4 | 2.4 | 40.2 | 1146 | 556 | 61 | 94.46 | 65.4 | 0.2 |
| | Disulfide bond | | | | | | | | | | | | | |
| 761 | Steric hindrance | T366Y/L368G/Y407Q | 557 | 61 | 62 | 5.9 | 5.9 | 88.2 | 1147 | 557 | 61 | 51.19 | 51.6 | 0.07 |
| 762 | Steric hindrance + | Q347R/K360E/D399I/F | 558 | 61 | 62 | 65.6 | 0.0 | 34.4 | 1148 | 558 | 61 | 96.74 | 65.4 | 0.2 |
| | Charge | 405T/K409Y | | | | | | | | | | | | |
| 763 | Steric hindrance + | E345R/Q347R/K360D/ | 559 | 61 | 62 | 61.2 | 0.0 | 38.8 | 1149 | 559 | 61 | 97.02 | 66.6 | 0.19 |
| | Charge | T366V/D399L/Y407A/ | | | | | | | | | | | | |
| | | K409L | | | | | | | | | | | | |
| 764 | Steric hindrance + | E345R/Q347R/K360D/ | 560 | 61 | 62 | 58.2 | 0.0 | 41.8 | 1150 | 560 | 61 | 97.69 | 68.4 | 0.2 |
| | Charge | T366V/D399I/Y407A/ | | | | | | | | | | | | |
| | | K409I | | | | | | | | | | | | |

TABLE 16
Amino acid modifications that showed strong heavy-chain homodimerization-promoting
ability with a heteromultimer ratio (%) of 10% or lower in Table 15

| Sample | | Modifications that promote formation of heavy chain |
|---|---|---|
| No. | Category of modification effect | homodimers |
| 766 | Charge | E356R/D399K/K409E/K439E |
| 767 | Charge | E356H/D399K/K409E/K439E |
| 768 | Charge | E356K/D399R/K409E/K439E |
| 769 | Charge | E356K/D399H/K409E/K439E |
| 770 | Charge | E356K/D399K/K409D/K439E |
| 771 | Charge | E356K/D399K/K409E/K439D |
| 772 | Charge | E356K/D399R/K409D/K439E |
| 773 | Charge | E356K/D399H/K409D/K439E |
| 774 | Charge | E356R/K392D/D399K/K439E |
| 775 | Charge | E356K/K392D/D399K/K439D |
| 776 | Charge | E356K/K392D/D399R/K439E |
| 777 | Charge | E356K/K392E/D399K/K439E |
| 778 | Charge | K392D/D399R/K409D |
| 779 | Charge | K392D/D399K/K409E |
| 780 | Charge | K392E/D399K/K409D |
| 781 | Charge | E356K/K392D/D399K/K409E |
| 782 | Charge | E356K/K392D/D399R/K409D |
| 783 | Charge | E356R/K392D/D399K/K409D |
| 784 | Charge | E356H/K392D/D399K/K409D |
| 785 | Charge | E356K/K392E/D399K/K409D |
| 786 | Charge | E356R/K392D/D399K/K439D |
| 787 | Charge | E356K/K392D/D399H/K409D |
| 788 | Charge | E356R/D399K/K409E/K439D |
| 789 | Charge | E356H/D399K/K409E/K439D |
| 790 | Steric hindrance + Charge | Q347R/K360D/T366V/D399M/Y407A/K409V |
| 791 | Steric hindrance + Charge | E345R/Q347K/K360D/T366V/D399M/Y407A/K409V |
| 792 | Steric hindrance + Charge | E345R/Q347R/K360E/T366V/D399M/Y407A/K409V |
| 793 | Steric hindrance + Charge | Q347K/K360D/T366V/D399M/Y407A/K409V |
| 794 | Steric hindrance + Charge | Q347R/K360E/T366V/D399M/Y407A/K409V |
| 795 | Steric hindrance + Charge | Q347K/K360E/T366V/D399M/Y407A/K409V |
| 796 | Steric hindrance + Charge | E345R/Q347R/K360D/T366V/D399Q/Y407A/K409Q |
| 797 | Steric hindrance + Charge | E345R/Q347R/K360D/T366V/D399N/Y407A/K409Q |
| 798 | Steric hindrance + Charge | E345R/Q347R/K360D/T366V/D399H/Y407A/K409Q |
| 799 | Steric hindrance + Charge | E345R/Q347R/K360D/T366V/D399I/Y407A/K409Q |
| 800 | Steric hindrance + Charge | E345R/Q347R/K360D/T366V/D399F/Y407A/K409Q |
| 801 | Steric hindrance + Charge | E345R/Q347R/K360D/T366V/D399Y/Y407A/K409Q |
| 802 | Steric hindrance + Charge | E345R/Q347R/K360D/T366V/D399T/Y407A/K409Q |
| 803 | Steric hindrance + Charge | E345R/Q347R/K360D/T366V/D399S/Y407A/K409Q |
| 808 | Steric hindrance + Charge | E345R/Q347R/K360D/T366V/D399F/Y407A/K409N |
| 812 | Steric hindrance + Charge | E345R/Q347R/K360D/T366V/D399Q/Y407A/K409H |
| 813 | Steric hindrance + Charge | E345R/Q347R/K360D/T366V/D399N/Y407A/K409H |
| 814 | Steric hindrance + Charge | E345R/Q347R/K360D/T366V/D399H/Y407A/K409H |
| 815 | Steric hindrance + Charge | E345R/Q347R/K360D/T366V/D399V/Y407A/K409H |
| 816 | Steric hindrance + Charge | E345R/Q347R/K360D/T366V/D399L/Y407A/K409H |
| 817 | Steric hindrance + Charge | E345R/Q347R/K360D/T366V/D399F/Y407A/K409H |
| 818 | Steric hindrance + Charge | E345R/Q347R/K360D/T366V/D399Y/Y407A/K409H |
| 819 | Steric hindrance + Charge | E345R/Q347R/K360D/T366V/D399T/Y407A/K409H |
| 820 | Steric hindrance + Charge | E345R/Q347R/K360D/T366V/D399S/Y407A/K409H |
| 821 | Steric hindrance + Charge | E345R/Q347R/K360D/T366V/D399Q/Y407A/K409V |
| 822 | Steric hindrance + Charge | E345R/Q347R/K360D/T366V/D399N/Y407A/K409V |
| 823 | Steric hindrance + Charge | E345R/Q347R/K360D/T366V/D399H/Y407A/K409V |
| 824 | Steric hindrance + Charge | E345R/Q347R/K360D/T366V/D399V/Y407A/K409V |
| 825 | Steric hindrance + Charge | E345R/Q347R/K360D/T366V/D399L/Y407A/K409V |
| 826 | Steric hindrance + Charge | E345R/Q347R/K360D/T366V/D399I/Y407A/K409V |
| 827 | Steric hindrance + Charge | E345R/Q347R/K360D/T366V/D399F/Y407A/K409V |
| 828 | Steric hindrance + Charge | E345R/Q347R/K360D/T366V/D399Y/Y407A/K409V |
| 829 | Steric hindrance + Charge | E345R/Q347R/K360D/T366V/D399T/Y407A/K409V |
| 830 | Steric hindrance + Charge | E345R/Q347R/K360D/T366V/D399S/Y407A/K409V |
| 831 | Steric hindrance + Charge | E345R/Q347R/K360D/T366V/D399Q/Y407A/K409L |
| 832 | Steric hindrance + Charge | E345R/Q347R/K360D/T366V/D399N/Y407A/K409L |
| 833 | Steric hindrance + Charge | E345R/Q347R/K360D/T366V/D399H/Y407A/K409L |
| 834 | Steric hindrance + Charge | E345R/Q347R/K360D/T366V/D399I/Y407A/K409L |
| 835 | Steric hindrance + Charge | E345R/Q347R/K360D/T366V/D399F/Y407A/K409L |
| 836 | Steric hindrance + Charge | E345R/Q347R/K360D/T366V/D399T/Y407A/K409L |
| 837 | Steric hindrance + Charge | E345R/Q347R/K360D/T366V/D399S/Y407A/K409L |
| 838 | Steric hindrance + Charge | E345R/Q347R/K360D/T366V/D399Q/Y407A/K409I |
| 839 | Steric hindrance + Charge | E345R/Q347R/K360D/T366V/D399N/Y407A/K409I |
| 840 | Steric hindrance + Charge | E345R/Q347R/K360D/T366V/D399H/Y407A/K409I |
| 841 | Steric hindrance + Charge | E345R/Q347R/K360D/T366V/D399V/Y407A/K409I |
| 842 | Steric hindrance + Charge | E345R/Q347R/K360D/T366V/D399L/Y407A/K409I |
| 843 | Steric hindrance + Charge | E345R/Q347R/K360D/T366V/D399F/Y407A/K409I |
| 844 | Steric hindrance + Charge | E345R/Q347R/K360D/T366V/D399Y/Y407A/K409I |
| 845 | Steric hindrance + Charge | E345R/Q347R/K360D/T366V/D399T/Y407A/K409I |
| 846 | Steric hindrance + Charge | E345R/Q347R/K360D/T366V/D399S/Y407A/K409I |
| 847 | Steric hindrance + Charge | E345R/Q347R/K360D/T366V/D399Q/Y407A/K409F |
| 848 | Steric hindrance + Charge | E345R/Q347R/K360D/T366V/D399N/Y407A/K409F |
| 849 | Steric hindrance + Charge | E345R/Q347R/K360D/T366V/D399H/Y407A/K409F |
| 850 | Steric hindrance + Charge | E345R/Q347R/K360D/T366V/D399V/Y407A/K409F |
| 851 | Steric hindrance + Charge | E345R/Q347R/K360D/T366V/D399L/Y407A/K409F |
| 852 | Steric hindrance + Charge | E345R/Q347R/K360D/T366V/D399I/Y407A/K409F |
| 853 | Steric hindrance + Charge | E345R/Q347R/K360D/T366V/D399F/Y407A/K409F |
| 854 | Steric hindrance + Charge | E345R/Q347R/K360D/T366V/D399Y/Y407A/K409F |
| 855 | Steric hindrance + Charge | E345R/Q347R/K360D/T366V/D399T/Y407A/K409F |
| 856 | Steric hindrance + Charge | E345R/Q347R/K360D/T366V/D399Q/Y407A/K409Y |
| 857 | Steric hindrance + Charge | E345R/Q347R/K360D/T366V/D399H/Y407A/K409Y |
| 858 | Steric hindrance + Charge | E345R/Q347R/K360D/T366V/D399V/Y407A/K409Y |
| 859 | Steric hindrance + Charge | E345R/Q347R/K360D/T366V/D399L/Y407A/K409Y |
| 860 | Steric hindrance + Charge | E345R/Q347R/K360D/T366V/D399I/Y407A/K409Y |
| 861 | Steric hindrance + Charge | E345R/Q347R/K360D/T366V/D399F/Y407A/K409Y |
| 862 | Steric hindrance + Charge | E345R/Q347R/K360D/T366V/D399Y/Y407A/K409Y |
| 863 | Steric hindrance + Charge | E345R/Q347R/K360D/T366V/D399S/Y407A/K409Y |
| 864 | Steric hindrance + Charge | E345R/Q347R/K360D/T366V/D399N/Y407A/K409T |
| 865 | Steric hindrance + Charge | E345R/Q347R/K360D/T366V/D399H/Y407A/K409T |
| 866 | Steric hindrance + Charge | E345R/Q347R/K360D/T366V/D399V/Y407A/K409T |
| 867 | Steric hindrance + Charge | E345R/Q347R/K360D/T366V/D399L/Y407A/K409T |
| 868 | Steric hindrance + Charge | E345R/Q347R/K360D/T366V/D399I/Y407A/K409T |
| 869 | Steric hindrance + Charge | E345R/Q347R/K360D/T366V/D399F/Y407A/K409T |
| 870 | Steric hindrance + Charge | E345R/Q347R/K360D/T366V/D399Y/Y407A/K409T |
| 871 | Steric hindrance + Charge | E345R/Q347R/K360D/T366V/D399T/Y407A/K409T |
| 872 | Steric hindrance + Charge | E345R/Q347R/K360D/T366V/D399S/Y407A/K409T |
| 873 | Steric hindrance + Charge | E345R/Q347R/K360D/T366V/D399Q/Y407A/K409S |
| 874 | Steric hindrance + Charge | E345R/Q347R/K360D/T366V/D399N/Y407A/K409S |
| 875 | Steric hindrance + Charge | E345R/Q347R/K360D/T366V/D399H/Y407A/K409S |
| 876 | Steric hindrance + Charge | E345R/Q347R/K360D/T366V/D399V/Y407A/K409S |
| 877 | Steric hindrance + Charge | E345R/Q347R/K360D/T366V/D399L/Y407A/K409S |
| 878 | Steric hindrance + Charge | E345R/Q347R/K360D/T366V/D399I/Y407A/K409S |
| 879 | Steric hindrance + Charge | E345R/Q347R/K360D/T366V/D399F/Y407A/K409S |
| 880 | Steric hindrance + Charge | E345R/Q347R/K360D/T366V/D399Y/Y407A/K409S |
| 881 | Steric hindrance + Charge | E345R/Q347R/K360D/T366V/D399T/Y407A/K409S |
| 882 | Steric hindrance + Charge | E345R/Q347R/K360D/T366V/D399S/Y407A/K409S |
| 883 | Steric hindrance + Charge | E345R/Q347R/K360D/T366V/D399M/Y407A/K409Q |
| 885 | Steric hindrance + Charge | E345R/Q347R/K360D/T366V/D399M/Y407A/K409H |
| 886 | Steric hindrance + Charge | E345R/Q347R/K360D/T366V/D399M/Y407A/K409L |
| 887 | Steric hindrance + Charge | E345R/Q347R/K360D/T366V/D399M/Y407A/K409I |
| 888 | Steric hindrance + Charge | E345R/Q347R/K360D/T366V/D399M/Y407A/K409F |
| 889 | Steric hindrance + Charge | E345R/Q347R/K360D/T366V/D399M/Y407A/K409Y |
| 890 | Steric hindrance + Charge | E345R/Q347R/K360D/T366V/D399M/Y407A/K409T |
| 891 | Steric hindrance + Charge | E345R/Q347R/K360D/T366V/D399M/Y407A/K409S |
| 892 | Steric hindrance + Charge | E345R/Q347R/K360D/T366V/D399V/Y407A/K409Q |
| 893 | Steric hindrance + Charge | E345R/Q347R/K360D/T366V/D399L/Y407A/K409Q |
| 894 | Steric hindrance + Charge | E345R/Q347R/K360D/T366V/D399I/Y407A/K409N |
| 895 | Steric hindrance + Charge | E345R/Q347R/K360D/T366V/D399I/Y407A/K409H |
| 896 | Steric hindrance + Charge | E345R/Q347R/K360D/T366V/D399V/Y407A/K409L |
| 897 | Steric hindrance + Charge | E345R/Q347R/K360D/T366V/D399Y/Y407A/K409L |
| 898 | Steric hindrance + Charge | E345R/Q347R/K360D/T366V/D399S/Y407A/K409F |
| 899 | Steric hindrance + Charge | E345R/Q347R/K360D/T366V/D399N/Y407A/K409Y |
| 900 | Steric hindrance + Charge | E345R/Q347R/K360D/T366V/D399T/Y407A/K409Y |
| 901 | Steric hindrance + Charge | E345R/Q347R/K360D/T366V/D399Q/Y407A/K409T |
| 903 | Steric hindrance + Charge | Q347R/K360E/D399V/F405T/K409F |
| 904 | Steric hindrance + Charge | Q347K/K360E/D399V/F405T/K409W |
| 905 | Steric hindrance + Charge | Q347Y/K360E/D399V/F405T/K409W |
| 906 | Steric hindrance + Charge | Q347R/K360D/D399V/F405T/K409W |
| 907 | Steric hindrance + Charge | Q347K/K360D/D399V/F405T/K409W |
| 908 | Steric hindrance + Charge | Q347H/K360D/D399V/F405T/K409W |
| 909 | Steric hindrance + Charge | Q347Y/K360D/D399V/F405T/K409W |
| 910 | Steric hindrance + Charge | Q347R/K360E/D399L/F405T/K409W |
| 911 | Steric hindrance + Charge | Q347R/K360E/D399I/F405T/K409W |
| 912 | Steric hindrance + Charge | Q347R/K360E/D399M/F405T/K409W |
| 913 | Steric hindrance + Charge | Q347R/K360E/D399S/F405T/K409W |
| 914 | Steric hindrance + Charge | Q347R/K360E/D399T/F405T/K409W |
| 915 | Steric hindrance + Charge + | Q347R/K360E/D399C/F405T/K409W |
| | Disulfide bond | |
| 916 | Steric hindrance + Charge | Q347R/K360E/D399H/F405T/K409W |
| 917 | Steric hindrance + Charge | Q347R/K360E/D399V/F405A/K409W |
| 918 | Steric hindrance + Charge | Q347R/K360E/D399V/F405V/K409W |
| 919 | Steric hindrance + Charge | Q347R/K360E/D399V/F405S/K409W |
| 920 | Steric hindrance + Charge + | Q347R/K360E/D399V/F405C/K409W |
| | Disulfide bond | |
| 921 | Steric hindrance + Charge | Q347R/K360E/D399V/F405N/K409W |
| 922 | Steric hindrance + Charge | Q347R/K360E/D399V/F405D/K409W |
| 923 | Steric hindrance + Charge | Q347R/K360E/D399V/F405T/K409Y |
| 924 | Steric hindrance + Charge | Q347R/K360E/D399V/F405T/K409H |
| 925 | Steric hindrance + Charge | Q347R/K360E/D399A/F405T/K409F |
| 926 | Steric hindrance + Charge | Q347R/K360E/D399L/F405T/K409F |
| 927 | Steric hindrance + Charge | Q347R/K360E/D399I/F405T/K409F |
| 928 | Steric hindrance + Charge | Q347R/K360E/D399M/F405T/K409F |
| 929 | Steric hindrance + Charge | Q347R/K360E/D399T/F405T/K409F |
| 930 | Steric hindrance + Charge + | Q347R/K360E/D399C/F405T/K409F |
| | Disulfide bond | |
| 931 | Steric hindrance + Charge | Q347R/K360E/D399N/F405T/K409F |
| 932 | Steric hindrance + Charge | Q347R/K360E/D399Q/F405T/K409F |
| 933 | Steric hindrance + Charge | Q347R/K360E/D399H/F405T/K409F |
| 934 | Steric hindrance + Charge | Q347R/K360E/D399A/F405T/K409Y |
| 935 | Steric hindrance + Charge | Q347R/K360E/D399L/F405T/K409Y |
| 936 | Steric hindrance + Charge | Q347R/K360E/D399M/F405T/K409Y |
| 937 | Steric hindrance + Charge | Q347R/K360E/D399S/F405T/K409Y |
| 938 | Steric hindrance + Charge | Q347R/K360E/D399T/F405T/K409Y |
| 939 | Steric hindrance + Charge + | Q347R/K360E/D399C/F405T/K409Y |
| | Disulfide bond | |
| 940 | Steric hindrance + Charge | Q347R/K360E/D399N/F405T/K409Y |
| 941 | Steric hindrance + Charge | Q347R/K360E/D399Q/F405T/K409Y |
| 942 | Steric hindrance + Charge | Q347R/K360E/D399H/F405T/K409Y |
| 943 | Steric hindrance + Charge | Q347R/K360E/D399V/F405A/K409F |
| 944 | Steric hindrance + Charge | Q347R/K360E/D399V/F405V/K409F |
| 945 | Steric hindrance + Charge | Q347R/K360E/D399V/F405S/K409F |
| 946 | Steric hindrance + Charge + | Q347R/K360E/D399V/F405C/K409F |
| | Disulfide bond | |
| 947 | Steric hindrance + Charge | Q347R/K360E/D399V/F405D/K409F |
| 948 | Steric hindrance + Charge | Q347R/K360E/D399V/F405A/K409Y |
| 949 | Steric hindrance + Charge + | Q347R/K360E/D399V/F405C/K409Y |
| | Disulfide bond | |
| 950 | Steric hindrance + Charge | Q347R/K360E/D399V/F405N/K409Y |
| 951 | Steric hindrance + Charge | Q347R/K360E/D399V/F405D/K409Y |
| 952 | Steric hindrance + Charge | Q347R/K360E/D399L/F405A/K409W |
| 953 | Steric hindrance + Charge | Q347R/K360E/D399L/F405S/K409W |
| 954 | Steric hindrance + Charge + | Q347R/K360E/D399L/F405C/K409W |
| | Disulfide bond | |
| 955 | Steric hindrance + Charge | Q347R/K360E/D399I/F405S/K409W |
| 956 | Steric hindrance + Charge + | Q347R/K360E/D399I/F405C/K409W |
| | Disulfide bond | |
| 957 | Steric hindrance + Charge | Q347R/K360E/D399M/F405A/K409W |
| 958 | Steric hindrance + Charge | Q347R/K360E/D399M/F405S/K409W |
| 959 | Steric hindrance + Charge + | Q347R/K360E/D399M/F405C/K409W |
| | Disulfide bond | |
| 960 | Steric hindrance + Charge | Q347R/K360E/D399G/F405T/K409W |
| 961 | Steric hindrance + Charge | Q347H/K360E/D399V/F405T/K409W |
| 962 | Steric hindrance + Charge | Q347R/K360E/D399A/F405T/K409W |
| 963 | Steric hindrance + Charge | Q347R/K360E/D399N/F405T/K409W |
| 964 | Steric hindrance + Charge | Q347R/K360E/D399Q/F405T/K409W |
| 965 | Steric hindrance + Charge | Q347R/K360E/D399S/F405T/K409F |
| 966 | Steric hindrance + Charge | Q347R/K360E/D399V/F405N/K409F |
| 967 | Steric hindrance + Charge | Q347R/K360E/D399V/F405V/K409Y |
| 968 | Steric hindrance + Charge | Q347R/K360E/D399V/F405S/K409Y |
| 969 | Steric hindrance + Charge | Q347R/K360E/D399I/F405A/K409W |
| 970 | Steric hindrance + Charge | Q347R/K360E/D399V/F405G/K409W |
| 971 | Steric hindrance | T394W/F405S |
| 972 | Steric hindrance | T394W/F405T |
| 973 | Steric hindrance | T394Y/F405A |
| 975 | Steric hindrance | T394Y/F405T |
| 976 | Steric hindrance + Disulfide bond | T394Y/F405C |
| 978 | Steric hindrance | T394F/F405G |
| 979 | Steric hindrance + Disulfide bond | T394F/F405C |
| 980 | Steric hindrance | T394F/F405V |
| 986 | Steric hindrance + Disulfide bond | T394W/F405C |
| 987 | Steric hindrance | T394W/F405V |
| 988 | Steric hindrance | T394Y/F405G |
| 989 | Steric hindrance | T394Y/F405S |
| 990 | Steric hindrance | T394F/F405S |
| 991 | Steric hindrance | T394F/F405T |
| 993 | Steric hindrance | T394W/F405G |
| 994 | Steric hindrance | T366W/L368S/Y407V |
| 995 | Steric hindrance | T366W/L368T/Y407V |
| 996 | Steric hindrance + Disulfide bond | T366W/L368C/Y407V |
| 997 | Steric hindrance | T366W/L368V/Y407V |
| 998 | Steric hindrance | T366W/L368G/Y407V |
| 999 | Steric hindrance | T366Y/L368A/Y407V |
| 1000 | Steric hindrance | T366F/L368A/Y407V |
| 1002 | Steric hindrance | T366W/L368A/Y407L |
| 1003 | Steric hindrance | T366W/L368A/Y407I |
| 1004 | Steric hindrance | T366W/L368A/Y407M |
| 1005 | Steric hindrance | T366W/L368A/Y407A |
| 1006 | Steric hindrance | T366W/L368A/Y407S |
| 1007 | Steric hindrance | T366W/L368A/Y407T |
| 1008 | Steric hindrance + Disulfide bond | T366W/L368A/Y407C |
| 1010 | Steric hindrance | T366W/L368A/Y407N |
| 1011 | Steric hindrance + Disulfide bond | T366W/L368C/Y407C |
| 1013 | Steric hindrance | T366W/L368S/Y407L |
| 1014 | Steric hindrance | T366W/L368T/Y407L |
| 1015 | Steric hindrance + Disulfide bond | T366W/L368C/Y407L |
| 1016 | Steric hindrance | T366W/L368V/Y407L |
| 1017 | Steric hindrance | T366W/L368G/Y407L |
| 1019 | Steric hindrance + Disulfide bond | T366W/L368C/Y407I |
| 1020 | Steric hindrance | T366W/L368V/Y407I |
| 1021 | Steric hindrance | T366W/L368G/Y407I |
| 1022 | Steric hindrance | T366W/L368S/Y407M |
| 1023 | Steric hindrance | T366W/L368T/Y407M |
| 1024 | Steric hindrance + Disulfide bond | T366W/L368C/Y407M |
| 1025 | Steric hindrance | T366W/L368V/Y407M |
| 1026 | Steric hindrance | T366W/L368G/Y407M |
| 1027 | Steric hindrance | T366W/L368S/Y407A |
| 1028 | Steric hindrance | T366W/L368T/Y407A |
| 1029 | Steric hindrance + Disulfide bond | T366W/L368C/Y407A |
| 1030 | Steric hindrance | T366W/L368V/Y407A |
| 1031 | Steric hindrance | T366W/L368G/Y407A |
| 1032 | Steric hindrance | T366W/L368S/Y407T |
| 1033 | Steric hindrance | T366W/L368T/Y407T |
| 1034 | Steric hindrance + Disulfide bond | T366W/L368C/Y407T |
| 1035 | Steric hindrance | T366W/L368V/Y407T |
| 1036 | Steric hindrance | T366W/L368G/Y407T |
| 1037 | Steric hindrance + Disulfide bond | T366W/L368S/Y407C |
| 1038 | Steric hindrance + Disulfide bond | T366W/L368T/Y407C |
| 1039 | Steric hindrance + Disulfide bond | T366W/L368V/Y407C |
| 1040 | Steric hindrance + Disulfide bond | T366W/L368G/Y407C |
| 1045 | Steric hindrance | T366W/L368S/Y407N |
| 1046 | Steric hindrance | T366W/L368T/Y407N |
| 1049 | Steric hindrance | T366W/L368S/Y407Q |
| 1050 | Steric hindrance | T366W/L368T/Y407Q |
| 1054 | Steric hindrance | T366Y/L368S/Y407L |
| 1055 | Steric hindrance + Disulfide bond | T366Y/L368C/Y407L |
| 1056 | Steric hindrance | T366Y/L368V/Y407L |
| 1058 | Steric hindrance | T366Y/L368S/Y407I |
| 1059 | Steric hindrance + Disulfide bond | T366Y/L368C/Y407I |
| 1060 | Steric hindrance | T366Y/L368V/Y407I |
| 1061 | Steric hindrance | T366Y/L368G/Y407I |
| 1062 | Steric hindrance | T366Y/L368G/Y407M |
| 1063 | Steric hindrance | T366Y/L368V/Y407A |
| 1065 | Steric hindrance | T366Y/L368S/Y407T |
| 1066 | Steric hindrance | T366Y/L368T/Y407T |
| 1067 | Steric hindrance + Disulfide bond | T366Y/L368C/Y407T |
| 1068 | Steric hindrance | T366Y/L368V/Y407T |
| 1070 | Steric hindrance + Disulfide bond | T366Y/L368T/Y407C |
| 1071 | Steric hindrance + Disulfide bond | T366Y/L368V/Y407C |
| 1078 | Steric hindrance | T366Y/L368S/Y407N |
| 1083 | Steric hindrance | T366Y/L368S/Y407Q |
| 1086 | Steric hindrance | T366W/L368A/Y407Q |
| 1087 | Steric hindrance | T366W/L368T/Y407I |
| 1091 | Steric hindrance + Disulfide bond | T366Y/L368C/Y407C |
| 1092 | Steric hindrance | T366F/L368S/Y407L |
| 1094 | Steric hindrance + Disulfide bond | T366F/L368C/Y407L |
| 1095 | Steric hindrance | T366F/L368V/Y407L |
| 1096 | Steric hindrance | T366F/L368G/Y407L |
| 1097 | Steric hindrance | T366F/L368S/Y407I |
| 1098 | Steric hindrance | T366F/L368T/Y407I |
| 1099 | Steric hindrance + Disulfide bond | T366F/L368C/Y407I |
| 1100 | Steric hindrance | T366F/L368V/Y407I |
| 1101 | Steric hindrance | T366F/L368G/Y407I |
| 1102 | Steric hindrance | T366F/L368S/Y407M |
| 1103 | Steric hindrance | T366F/L368T/Y407M |
| 1104 | Steric hindrance + Disulfide bond | T366F/L368C/Y407M |
| 1105 | Steric hindrance | T366F/L368V/Y407M |
| 1107 | Steric hindrance | T366F/L368S/Y407A |
| 1108 | Steric hindrance | T366F/L368T/Y407A |
| 1109 | Steric hindrance + Disulfide bond | T366F/L368C/Y407A |
| 1110 | Steric hindrance | T366F/L368V/Y407A |
| 1112 | Steric hindrance | T366F/L368S/Y407T |
| 1113 | Steric hindrance | T366F/L368T/Y407T |
| 1114 | Steric hindrance + Disulfide bond | T366F/L368C/Y407T |
| 1115 | Steric hindrance | T366F/L368V/Y407T |
| 1117 | Steric hindrance + Disulfide bond | T366F/L368S/Y407C |
| 1118 | Steric hindrance + Disulfide bond | T366F/L368T/Y407C |
| 1119 | Steric hindrance + Disulfide bond | T366F/L368V/Y407C |
| 1134 | Steric hindrance + Disulfide bond | T366F/L368C/Y407C |
| 1135 | Steric hindrance | T366Y/L368T/Y407I |
| 1138 | Steric hindrance + Disulfide bond | T366Y/L368S/Y407C |
| 1140 | Steric hindrance | T366Y/L368S/Y407M |
| 1141 | Steric hindrance | T366Y/L368T/Y407M |
| 1142 | Steric hindrance + Disulfide bond | T366Y/L368C/Y407M |
| 1143 | Steric hindrance | T366Y/L368V/Y407M |
| 1144 | Steric hindrance | T366Y/L368S/Y407A |
| 1145 | Steric hindrance | T366Y/L368T/Y407A |
| 1146 | Steric hindrance + Disulfide bond | T366Y/L368C/Y407A |
| 1147 | Steric hindrance | T366Y/L368G/Y407Q |
| 1148 | Steric hindrance + Charge | Q347R/K360E/D399I/F405T/K409Y |
| 1149 | Steric hindrance + Charge | E345R/Q347R/K360D/T366V/D399L/Y407A/K409L |
| 1150 | Steric hindrance + Charge | E345R/Q347R/K360D/T366V/D399I/Y407A/K409I |

TABLE 17
Amino acid modifications that showed strong heavy-chain homodimerization-
promoting ability with a heteromultimer ratio (%) of 0% in Table 15

| Sample | | Modifications that promote formation of heavy chain |
|---|---|---|
| No. | Category of modification effect | homodimers |
| 766 | Charge | E356R/D399K/K409E/K439E |
| 768 | Charge | E356K/D399R/K409E/K439E |
| 769 | Charge | E356K/D399H/K409E/K439E |
| 770 | Charge | E356K/D399K/K409D/K439E |
| 771 | Charge | E356K/D399K/K409E/K439D |
| 772 | Charge | E356K/D399R/K409D/K439E |
| 779 | Charge | K392D/D399K/K409E |
| 781 | Charge | E356K/K392D/D399K/K409E |
| 782 | Charge | E356K/K392D/D399R/K409D |
| 783 | Charge | E356R/K392D/D399K/K409D |
| 784 | Charge | E356H/K392D/D399K/K409D |
| 785 | Charge | E356K/K392E/D399K/K409D |
| 787 | Charge | E356K/K392D/D399H/K409D |
| 788 | Charge | E356R/D399K/K409E/K439D |
| 790 | Steric hindrance + Charge | Q347R/K360D/T366V/D399M/Y407A/K409V |
| 791 | Steric hindrance + Charge | E345R/Q347K/K360D/T366V/D399M/Y407A/K409V |
| 792 | Steric hindrance + Charge | E345R/Q347R/K360E/T366V/D399M/Y407A/K409V |
| 793 | Steric hindrance + Charge | Q347K/K360D/T366V/D399M/Y407A/K409V |
| 794 | Steric hindrance + Charge | Q347R/K360E/T366V/D399M/Y407A/K409V |
| 795 | Steric hindrance + Charge | Q347K/K360E/T366V/D399M/Y407A/K409V |
| 797 | Steric hindrance + Charge | E345R/Q347R/K360D/T366V/D399N/Y407A/K409Q |
| 798 | Steric hindrance + Charge | E345R/Q347R/K360D/T366V/D399H/Y407A/K409Q |
| 799 | Steric hindrance + Charge | E345R/Q347R/K360D/T366V/D399I/Y407A/K409Q |
| 800 | Steric hindrance + Charge | E345R/Q347R/K360D/T366V/D399F/Y407A/K409Q |
| 801 | Steric hindrance + Charge | E345R/Q347R/K360D/T366V/D399Y/Y407A/K409Q |
| 802 | Steric hindrance + Charge | E345R/Q347R/K360D/T366V/D399T/Y407A/K409Q |
| 803 | Steric hindrance + Charge | E345R/Q347R/K360D/T366V/D399S/Y407A/K409Q |
| 815 | Steric hindrance + Charge | E345R/Q347R/K360D/T366V/D399V/Y407A/K409H |
| 816 | Steric hindrance + Charge | E345R/Q347R/K360D/T366V/D399L/Y407A/K409H |
| 819 | Steric hindrance + Charge | E345R/Q347R/K360D/T366V/D399T/Y407A/K409H |
| 820 | Steric hindrance + Charge | E345R/Q347R/K360D/T366V/D399S/Y407A/K409H |
| 821 | Steric hindrance + Charge | E345R/Q347R/K360D/T366V/D399Q/Y407A/K409V |
| 822 | Steric hindrance + Charge | E345R/Q347R/K360D/T366V/D399N/Y407A/K409V |
| 823 | Steric hindrance + Charge | E345R/Q347R/K360D/T366V/D399H/Y407A/K409V |
| 824 | Steric hindrance + Charge | E345R/Q347R/K360D/T366V/D399V/Y407A/K409V |
| 825 | Steric hindrance + Charge | E345R/Q347R/K360D/T366V/D399L/Y407A/K409V |
| 826 | Steric hindrance + Charge | E345R/Q347R/K360D/T366V/D399I/Y407A/K409V |
| 827 | Steric hindrance + Charge | E345R/Q347R/K360D/T366V/D399F/Y407A/K409V |
| 828 | Steric hindrance + Charge | E345R/Q347R/K360D/T366V/D399Y/Y407A/K409V |
| 829 | Steric hindrance + Charge | E345R/Q347R/K360D/T366V/D399T/Y407A/K409V |
| 830 | Steric hindrance + Charge | E345R/Q347R/K360D/T366V/D399S/Y407A/K409V |
| 831 | Steric hindrance + Charge | E345R/Q347R/K360D/T366V/D399Q/Y407A/K409L |
| 832 | Steric hindrance + Charge | E345R/Q347R/K360D/T366V/D399N/Y407A/K409L |
| 833 | Steric hindrance + Charge | E345R/Q347R/K360D/T366V/D399H/Y407A/K409L |
| 834 | Steric hindrance + Charge | E345R/Q347R/K360D/T366V/D399I/Y407A/K409L |
| 835 | Steric hindrance + Charge | E345R/Q347R/K360D/T366V/D399F/Y407A/K409L |
| 836 | Steric hindrance + Charge | E345R/Q347R/K360D/T366V/D399T/Y407A/K409L |
| 837 | Steric hindrance + Charge | E345R/Q347R/K360D/T366V/D399S/Y407A/K409L |
| 838 | Steric hindrance + Charge | E345R/Q347R/K360D/T366V/D399Q/Y407A/K409I |
| 839 | Steric hindrance + Charge | E345R/Q347R/K360D/T366V/D399N/Y407A/K409I |
| 840 | Steric hindrance + Charge | E345R/Q347R/K360D/T366V/D399H/Y407A/K409I |
| 841 | Steric hindrance + Charge | E345R/Q347R/K360D/T366V/D399V/Y407A/K409I |
| 842 | Steric hindrance + Charge | E345R/Q347R/K360D/T366V/D399L/Y407A/K409I |
| 843 | Steric hindrance + Charge | E345R/Q347R/K360D/T366V/D399F/Y407A/K409I |
| 844 | Steric hindrance + Charge | E345R/Q347R/K360D/T366V/D399Y/Y407A/K409I |
| 845 | Steric hindrance + Charge | E345R/Q347R/K360D/T366V/D399T/Y407A/K409I |
| 846 | Steric hindrance + Charge | E345R/Q347R/K360D/T366V/D399S/Y407A/K409I |
| 847 | Steric hindrance + Charge | E345R/Q347R/K360D/T366V/D399Q/Y407A/K409F |
| 848 | Steric hindrance + Charge | E345R/Q347R/K360D/T366V/D399N/Y407A/K409F |
| 849 | Steric hindrance + Charge | E345R/Q347R/K360D/T366V/D399H/Y407A/K409F |
| 850 | Steric hindrance + Charge | E345R/Q347R/K360D/T366V/D399V/Y407A/K409F |
| 851 | Steric hindrance + Charge | E345R/Q347R/K360D/T366V/D399L/Y407A/K409F |
| 852 | Steric hindrance + Charge | E345R/Q347R/K360D/T366V/D399I/Y407A/K409F |
| 853 | Steric hindrance + Charge | E345R/Q347R/K360D/T366V/D399F/Y407A/K409F |
| 854 | Steric hindrance + Charge | E345R/Q347R/K360D/T366V/D399Y/Y407A/K409F |
| 855 | Steric hindrance + Charge | E345R/Q347R/K360D/T366V/D399T/Y407A/K409F |
| 858 | Steric hindrance + Charge | E345R/Q347R/K360D/T366V/D399V/Y407A/K409Y |
| 859 | Steric hindrance + Charge | E345R/Q347R/K360D/T366V/D399L/Y407A/K409Y |
| 860 | Steric hindrance + Charge | E345R/Q347R/K360D/T366V/D399I/Y407A/K409Y |
| 861 | Steric hindrance + Charge | E345R/Q347R/K360D/T366V/D399F/Y407A/K409Y |
| 863 | Steric hindrance + Charge | E345R/Q347R/K360D/T366V/D399S/Y407A/K409Y |
| 864 | Steric hindrance + Charge | E345R/Q347R/K360D/T366V/D399N/Y407A/K409T |
| 866 | Steric hindrance + Charge | E345R/Q347R/K360D/T366V/D399V/Y407A/K409T |
| 867 | Steric hindrance + Charge | E345R/Q347R/K360D/T366V/D399L/Y407A/K409T |
| 868 | Steric hindrance + Charge | E345R/Q347R/K360D/T366V/D399I/Y407A/K409T |
| 871 | Steric hindrance + Charge | E345R/Q347R/K360D/T366V/D399T/Y407A/K409T |
| 872 | Steric hindrance + Charge | E345R/Q347R/K360D/T366V/D399S/Y407A/K409T |
| 873 | Steric hindrance + Charge | E345R/Q347R/K360D/T366V/D399Q/Y407A/K409S |
| 874 | Steric hindrance + Charge | E345R/Q347R/K360D/T366V/D399N/Y407A/K409S |
| 875 | Steric hindrance + Charge | E345R/Q347R/K360D/T366V/D399H/Y407A/K409S |
| 876 | Steric hindrance + Charge | E345R/Q347R/K360D/T366V/D399V/Y407A/K409S |
| 877 | Steric hindrance + Charge | E345R/Q347R/K360D/T366V/D399L/Y407A/K409S |
| 878 | Steric hindrance + Charge | E345R/Q347R/K360D/T366V/D399I/Y407A/K409S |
| 879 | Steric hindrance + Charge | E345R/Q347R/K360D/T366V/D399F/Y407A/K409S |
| 880 | Steric hindrance + Charge | E345R/Q347R/K360D/T366V/D399Y/Y407A/K409S |
| 881 | Steric hindrance + Charge | E345R/Q347R/K360D/T366V/D399T/Y407A/K409S |
| 882 | Steric hindrance + Charge | E345R/Q347R/K360D/T366V/D399S/Y407A/K409S |
| 883 | Steric hindrance + Charge | E345R/Q347R/K360D/T366V/D399M/Y407A/K409Q |
| 886 | Steric hindrance + Charge | E345R/Q347R/K360D/T366V/D399M/Y407A/K409L |
| 887 | Steric hindrance + Charge | E345R/Q347R/K360D/T366V/D399M/Y407A/K409I |
| 888 | Steric hindrance + Charge | E345R/Q347R/K360D/T366V/D399M/Y407A/K409F |
| 889 | Steric hindrance + Charge | E345R/Q347R/K360D/T366V/D399M/Y407A/K409Y |
| 890 | Steric hindrance + Charge | E345R/Q347R/K360D/T366V/D399M/Y407A/K409T |
| 891 | Steric hindrance + Charge | E345R/Q347R/K360D/T366V/D399M/Y407A/K409S |
| 892 | Steric hindrance + Charge | E345R/Q347R/K360D/T366V/D399V/Y407A/K409Q |
| 893 | Steric hindrance + Charge | E345R/Q347R/K360D/T366V/D399L/Y407A/K409Q |
| 895 | Steric hindrance + Charge | E345R/Q347R/K360D/T366V/D399I/Y407A/K409H |
| 896 | Steric hindrance + Charge | E345R/Q347R/K360D/T366V/D399V/Y407A/K409L |
| 897 | Steric hindrance + Charge | E345R/Q347R/K360D/T366V/D399Y/Y407A/K409L |
| 898 | Steric hindrance + Charge | E345R/Q347R/K360D/T366V/D399S/Y407A/K409F |
| 900 | Steric hindrance + Charge | E345R/Q347R/K360D/T366V/D399T/Y407A/K409Y |
| 901 | Steric hindrance + Charge | E345R/Q347R/K360D/T366V/D399Q/Y407A/K409T |
| 903 | Steric hindrance + Charge | Q347R/K360E/D399V/F405T/K409F |
| 904 | Steric hindrance + Charge | Q347K/K360E/D399V/F405T/K409W |
| 905 | Steric hindrance + Charge | Q347Y/K360E/D399V/F405T/K409W |
| 906 | Steric hindrance + Charge | Q347R/K360D/D399V/F405T/K409W |
| 907 | Steric hindrance + Charge | Q347K/K360D/D399V/F405T/K409W |
| 908 | Steric hindrance + Charge | Q347H/K360D/D399V/F405T/K409W |
| 910 | Steric hindrance + Charge | Q347R/K360E/D399L/F405T/K409W |
| 911 | Steric hindrance + Charge | Q347R/K360E/D399I/F405T/K409W |
| 912 | Steric hindrance + Charge | Q347R/K360E/D399M/F405T/K409W |
| 913 | Steric hindrance + Charge | Q347R/K360E/D399S/F405T/K409W |
| 914 | Steric hindrance + Charge | Q347R/K360E/D399T/F405T/K409W |
| 915 | Steric hindrance + Charge + | Q347R/K360E/D399C/F405T/K409W |
| | Disulfide bond | |
| 916 | Steric hindrance + Charge | Q347R/K360E/D399H/F405T/K409W |
| 917 | Steric hindrance + Charge | Q347R/K360E/D399V/F405A/K409W |
| 918 | Steric hindrance + Charge | Q347R/K360E/D399V/F405V/K409W |
| 926 | Steric hindrance + Charge | Q347R/K360E/D399L/F405T/K409F |
| 927 | Steric hindrance + Charge | Q347R/K360E/D399I/F405T/K409F |
| 928 | Steric hindrance + Charge | Q347R/K360E/D399M/F405T/K409F |
| 929 | Steric hindrance + Charge | Q347R/K360E/D399T/F405T/K409F |
| 931 | Steric hindrance + Charge | Q347R/K360E/D399N/F405T/K409F |
| 943 | Steric hindrance + Charge | Q347R/K360E/D399V/F405A/K409F |
| 944 | Steric hindrance + Charge | Q347R/K360E/D399V/F405V/K409F |
| 952 | Steric hindrance + Charge | Q347R/K360E/D399L/F405A/K409W |
| 957 | Steric hindrance + Charge | Q347R/K360E/D399M/F405A/K409W |
| 960 | Steric hindrance + Charge | Q347R/K360E/D399G/F405T/K409W |
| 961 | Steric hindrance + Charge | Q347H/K360E/D399V/F405T/K409W |
| 962 | Steric hindrance + Charge | Q347R/K360E/D399A/F405T/K409W |
| 963 | Steric hindrance + Charge | Q347R/K360E/D399N/F405T/K409W |
| 969 | Steric hindrance + Charge | Q347R/K360E/D399I/F405A/K409W |
| 971 | Steric hindrance | T394W/F405S |
| 972 | Steric hindrance | T394W/F405T |
| 993 | Steric hindrance | T394W/F405G |
| 996 | Steric hindrance + Disulfide bond | T366W/L368C/Y407V |
| 998 | Steric hindrance | T366W/L368G/Y407V |
| 999 | Steric hindrance | T366Y/L368A/Y407V |
| 1000 | Steric hindrance | T366F/L368A/Y407V |
| 1002 | Steric hindrance | T366W/L368A/Y407L |
| 1011 | Steric hindrance + Disulfide bond | T366W/L368C/Y407C |
| 1031 | Steric hindrance | T366W/L368G/Y407A |
| 1063 | Steric hindrance | T366Y/L368V/Y407A |
| 1087 | Steric hindrance | T366W/L368T/Y407I |
| 1095 | Steric hindrance | T366F/L368V/Y407L |
| 1100 | Steric hindrance | T366F/L368V/Y407I |
| 1105 | Steric hindrance | T366F/L368V/Y407M |
| 1134 | Steric hindrance + Disulfide bond | T366F/L368C/Y407C |
| 1143 | Steric hindrance | T366Y/L368V/Y407M |
| 1148 | Steric hindrance + Charge | Q347R/K360E/D399I/F405T/K409Y |
| 1149 | Steric hindrance + Charge | E345R/Q347R/K360D/T366V/D399L/Y407A/K409L |
| 1150 | Steric hindrance + Charge | E345R/Q347R/K360D/T366V/D399I/Y407A/K409I |

TABLE 18
Amino acid modifications that showed a heteromultimer ratio (%)
of 0%, a homodimer monomer ratio of 95% or higher, a Tm of 65°
C. or higher, and a yield (mg) of 0.15 mg or more in Table 15

| Sample | | Modifications that promote formation of heavy chain |
|---|---|---|
| No. | Category of modification effect | homodimers |
| 769 | Charge | E356K/D399H/K409E/K439E |
| 772 | Charge | E356K/D399R/K409D/K439E |
| 779 | Charge | K392D/D399K/K409E |
| 791 | Steric hindrance + Charge | E345R/Q347K/K360D/T366V/D399M/Y407A/K409V |
| 792 | Steric hindrance + Charge | E345R/Q347R/K360E/T366V/D399M/Y407A/K409V |
| 793 | Steric hindrance + Charge | Q347K/K360D/T366V/D399M/Y407A/K409V |
| 794 | Steric hindrance + Charge | Q347R/K360E/T366V/D399M/Y407A/K409V |
| 795 | Steric hindrance + Charge | Q347K/K360E/T366V/D399M/Y407A/K409V |
| 798 | Steric hindrance + Charge | E345R/Q347R/K360D/T366V/D399H/Y407A/K409Q |
| 799 | Steric hindrance + Charge | E345R/Q347R/K360D/T366V/D399I/Y407A/K409Q |
| 802 | Steric hindrance + Charge | E345R/Q347R/K360D/T366V/D399T/Y407A/K409Q |
| 803 | Steric hindrance + Charge | E345R/Q347R/K360D/T366V/D399S/Y407A/K409Q |
| 815 | Steric hindrance + Charge | E345R/Q347R/K360D/T366V/D399V/Y407A/K409H |
| 819 | Steric hindrance + Charge | E345R/Q347R/K360D/T366V/D399T/Y407A/K409H |
| 820 | Steric hindrance + Charge | E345R/Q347R/K360D/T366V/D399S/Y407A/K409H |
| 821 | Steric hindrance + Charge | E345R/Q347R/K360D/T366V/D399Q/Y407A/K409V |
| 822 | Steric hindrance + Charge | E345R/Q347R/K360D/T366V/D399N/Y407A/K409V |
| 823 | Steric hindrance + Charge | E345R/Q347R/K360D/T366V/D399H/Y407A/K409V |
| 824 | Steric hindrance + Charge | E345R/Q347R/K360D/T366V/D399V/Y407A/K409V |
| 825 | Steric hindrance + Charge | E345R/Q347R/K360D/T366V/D399L/Y407A/K409V |
| 826 | Steric hindrance + Charge | E345R/Q347R/K360D/T366V/D399I/Y407A/K409V |
| 827 | Steric hindrance + Charge | E345R/Q347R/K360D/T366V/D399F/Y407A/K409V |
| 828 | Steric hindrance + Charge | E345R/Q347R/K360D/T366V/D399Y/Y407A/K409V |
| 829 | Steric hindrance + Charge | E345R/Q347R/K360D/T366V/D399T/Y407A/K409V |
| 830 | Steric hindrance + Charge | E345R/Q347R/K360D/T366V/D399S/Y407A/K409V |
| 831 | Steric hindrance + Charge | E345R/Q347R/K360D/T366V/D399Q/Y407A/K409L |
| 832 | Steric hindrance + Charge | E345R/Q347R/K360D/T366V/D399N/Y407A/K409L |
| 833 | Steric hindrance + Charge | E345R/Q347R/K360D/T366V/D399H/Y407A/K409L |
| 834 | Steric hindrance + Charge | E345R/Q347R/K360D/T366V/D399I/Y407A/K409L |
| 835 | Steric hindrance + Charge | E345R/Q347R/K360D/T366V/D399F/Y407A/K409L |
| 836 | Steric hindrance + Charge | E345R/Q347R/K360D/T366V/D399T/Y407A/K409L |
| 837 | Steric hindrance + Charge | E345R/Q347R/K360D/T366V/D399S/Y407A/K409L |
| 838 | Steric hindrance + Charge | E345R/Q347R/K360D/T366V/D399Q/Y407A/K409I |
| 839 | Steric hindrance + Charge | E345R/Q347R/K360D/T366V/D399N/Y407A/K409I |
| 840 | Steric hindrance + Charge | E345R/Q347R/K360D/T366V/D399H/Y407A/K409I |
| 841 | Steric hindrance + Charge | E345R/Q347R/K360D/T366V/D399V/Y407A/K409I |
| 842 | Steric hindrance + Charge | E345R/Q347R/K360D/T366V/D399L/Y407A/K409I |
| 844 | Steric hindrance + Charge | E345R/Q347R/K360D/T366V/D399Y/Y407A/K409I |
| 845 | Steric hindrance + Charge | E345R/Q347R/K360D/T366V/D399T/Y407A/K409I |
| 846 | Steric hindrance + Charge | E345R/Q347R/K360D/T366V/D399S/Y407A/K409I |
| 847 | Steric hindrance + Charge | E345R/Q347R/K360D/T366V/D399Q/Y407A/K409F |
| 848 | Steric hindrance + Charge | E345R/Q347R/K360D/T366V/D399N/Y407A/K409F |
| 849 | Steric hindrance + Charge | E345R/Q347R/K360D/T366V/D399H/Y407A/K409F |
| 850 | Steric hindrance + Charge | E345R/Q347R/K360D/T366V/D399V/Y407A/K409F |
| 851 | Steric hindrance + Charge | E345R/Q347R/K360D/T366V/D399L/Y407A/K409F |
| 852 | Steric hindrance + Charge | E345R/Q347R/K360D/T366V/D399I/Y407A/K409F |
| 855 | Steric hindrance + Charge | E345R/Q347R/K360D/T366V/D399T/Y407A/K409F |
| 858 | Steric hindrance + Charge | E345R/Q347R/K360D/T366V/D399V/Y407A/K409Y |
| 863 | Steric hindrance + Charge | E345R/Q347R/K360D/T366V/D399S/Y407A/K409Y |
| 864 | Steric hindrance + Charge | E345R/Q347R/K360D/T366V/D399N/Y407A/K409T |
| 866 | Steric hindrance + Charge | E345R/Q347R/K360D/T366V/D399V/Y407A/K409T |
| 871 | Steric hindrance + Charge | E345R/Q347R/K360D/T366V/D399T/Y407A/K409T |
| 872 | Steric hindrance + Charge | E345R/Q347R/K360D/T366V/D399S/Y407A/K409T |
| 873 | Steric hindrance + Charge | E345R/Q347R/K360D/T366V/D399Q/Y407A/K409S |
| 874 | Steric hindrance + Charge | E345R/Q347R/K360D/T366V/D399N/Y407A/K409S |
| 875 | Steric hindrance + Charge | E345R/Q347R/K360D/T366V/D399H/Y407A/K409S |
| 876 | Steric hindrance + Charge | E345R/Q347R/K360D/T366V/D399V/Y407A/K409S |
| 877 | Steric hindrance + Charge | E345R/Q347R/K360D/T366V/D399L/Y407A/K409S |
| 878 | Steric hindrance + Charge | E345R/Q347R/K360D/T366V/D399I/Y407A/K409S |
| 881 | Steric hindrance + Charge | E345R/Q347R/K360D/T366V/D399T/Y407A/K409S |
| 883 | Steric hindrance + Charge | E345R/Q347R/K360D/T366V/D399M/Y407A/K409Q |
| 886 | Steric hindrance + Charge | E345R/Q347R/K360D/T366V/D399M/Y407A/K409L |
| 887 | Steric hindrance + Charge | E345R/Q347R/K360D/T366V/D399M/Y407A/K409I |
| 888 | Steric hindrance + Charge | E345R/Q347R/K360D/T366V/D399M/Y407A/K409F |
| 889 | Steric hindrance + Charge | E345R/Q347R/K360D/T366V/D399M/Y407A/K409Y |
| 890 | Steric hindrance + Charge | E345R/Q347R/K360D/T366V/D399M/Y407A/K409T |
| 891 | Steric hindrance + Charge | E345R/Q347R/K360D/T366V/D399M/Y407A/K409S |
| 892 | Steric hindrance + Charge | E345R/Q347R/K360D/T366V/D399V/Y407A/K409Q |
| 896 | Steric hindrance + Charge | E345R/Q347R/K360D/T366V/D399V/Y407A/K409L |
| 897 | Steric hindrance + Charge | E345R/Q347R/K360D/T366V/D399Y/Y407A/K409L |
| 898 | Steric hindrance + Charge | E345R/Q347R/K360D/T366V/D399S/Y407A/K409F |
| 900 | Steric hindrance + Charge | E345R/Q347R/K360D/T366V/D399T/Y407A/K409Y |
| 901 | Steric hindrance + Charge | E345R/Q347R/K360D/T366V/D399Q/Y407A/K409T |
| 903 | Steric hindrance + Charge | Q347R/K360E/D399V/F405T/K409F |
| 904 | Steric hindrance + Charge | Q347K/K360E/D399V/F405T/K409W |
| 905 | Steric hindrance + Charge | Q347Y/K360E/D399V/F405T/K409W |
| 906 | Steric hindrance + Charge | Q347R/K360D/D399V/F405T/K409W |
| 907 | Steric hindrance + Charge | Q347K/K360D/D399V/F405T/K409W |
| 908 | Steric hindrance + Charge | Q347H/K360D/D399V/F405T/K409W |
| 910 | Steric hindrance + Charge | Q347R/K360E/D399L/F405T/K409W |
| 911 | Steric hindrance + Charge | Q347R/K360E/D399I/F405T/K409W |
| 912 | Steric hindrance + Charge | Q347R/K360E/D399M/F405T/K409W |
| 913 | Steric hindrance + Charge | Q347R/K360E/D399S/F405T/K409W |
| 914 | Steric hindrance + Charge | Q347R/K360E/D399T/F405T/K409W |
| 915 | Steric hindrance + Charge + | Q347R/K360E/D399C/F405T/K409W |
| | Disulfide bond | |
| 916 | Steric hindrance + Charge | Q347R/K360E/D399H/F405T/K409W |
| 917 | Steric hindrance + Charge | Q347R/K360E/D399V/F405A/K409W |
| 918 | Steric hindrance + Charge | Q347R/K360E/D399V/F405V/K409W |
| 927 | Steric hindrance + Charge | Q347R/K360E/D399I/F405T/K409F |
| 928 | Steric hindrance + Charge | Q347R/K360E/D399M/F405T/K409F |
| 929 | Steric hindrance + Charge | Q347R/K360E/D399T/F405T/K409F |
| 931 | Steric hindrance + Charge | Q347R/K360E/D399N/F405T/K409F |
| 943 | Steric hindrance + Charge | Q347R/K360E/D399V/F405A/K409F |
| 944 | Steric hindrance + Charge | Q347R/K360E/D399V/F405V/K409F |
| 952 | Steric hindrance + Charge | Q347R/K360E/D399L/F405A/K409W |
| 957 | Steric hindrance + Charge | Q347R/K360E/D399M/F405A/K409W |
| 960 | Steric hindrance + Charge | Q347R/K360E/D399G/F405T/K409W |
| 961 | Steric hindrance + Charge | Q347H/K360E/D399V/F405T/K409W |
| 963 | Steric hindrance + Charge | Q347R/K360E/D399N/F405T/K409W |
| 969 | Steric hindrance + Charge | Q347R/K360E/D399I/F405A/K409W |
| 971 | Steric hindrance | T394W/F405S |
| 972 | Steric hindrance | T394W/F405T |
| 993 | Steric hindrance | T394W/F405G |
| 996 | Steric hindrance + Disulfide bond | T366W/L368C/Y407V |
| 998 | Steric hindrance | T366W/L368G/Y407V |
| 999 | Steric hindrance | T366Y/L368A/Y407V |
| 1000 | Steric hindrance | T366F/L368A/Y407V |
| 1002 | Steric hindrance | T366W/L368A/Y407L |
| 1011 | Steric hindrance + Disulfide bond | T366W/L368C/Y407C |
| 1031 | Steric hindrance | T366W/L368G/Y407A |
| 1063 | Steric hindrance | T366Y/L368V/Y407A |
| 1087 | Steric hindrance | T366W/L368T/Y407I |
| 1095 | Steric hindrance | T366F/L368V/Y407L |
| 1105 | Steric hindrance | T366F/L368V/Y407M |
| 1134 | Steric hindrance + Disulfide bond | T366F/L368C/Y407C |
| 1143 | Steric hindrance | T366Y/L368V/Y407M |
| 1148 | Steric hindrance + Charge | Q347R/K360E/D399I/F405T/K409Y |
| 1149 | Steric hindrance + Charge | E345R/Q347R/K360D/T366V/D399L/Y407A/K409L |
| 1150 | Steric hindrance + Charge | E345R/Q347R/K360D/T366V/D399I/Y407A/K409I |

TABLE 19
Combinations of modifications that showed strong suppression of heteromeric association in Table 1, and amino acid
modifications at the respective residue positions that suppressed the heteromultimer ratio below 51.7% in Table 15

| | Modification set No. | | | | | | | | | | | | | | | | |
|---|---|---|---|---|---|---|---|---|---|---|---|---|---|---|---|---|---|
| | 1 | | 2 | | | 3 | | | | | | 4 | | | | | |
| | EU numbering | | | | | | | | | | | | | | | | |
| | 394 | 405 | 366 | 368 | 407 | 347 | 360 | 399 | 405 | 409 | 345 | 347 | 360 | 366 | 399 | 407 | 409 |
| Combinations | W | A | W | A | V | R | E | V | T | W | R | R | D | V | M | A | V |
| of | | | | | | | | | | | | | | | | | |
| modifications | | | | | | | | | | | | | | | | | |
| that showed | | | | | | | | | | | | | | | | | |
| strong | | | | | | | | | | | | | | | | | |
| suppression | | | | | | | | | | | | | | | | | |
| of | | | | | | | | | | | | | | | | | |
| heteromeric | | | | | | | | | | | | | | | | | |
| association in | | | | | | | | | | | | | | | | | |
| Table 1 | | | | | | | | | | | | | | | | | |
| Amino acid | F | S | Y | S | L | K | D | L | A | F | E | K | E | | Q | | Q |
| modifications | Y | T | F | T | I | Y | | I | V | Y | | | | | N | | N |
| in Table 15 | H | C | | C | M | H | | M | S | H | | | | | H | | H |
| that | | V | | V | A | | | S | C | | | | | | I | | L |
| suppressed | | G | | G | S | | | T | N | | | | | | F | | I |
| the hetero- | | | | | T | | | C | D | | | | | | Y | | F |
| multimer | | | | | C | | | H | G | | | | | | T | | Y |
| ratio below | | | | | H | | | A | | | | | | | S | | T |
| that of WT, | | | | | N | | | N | | | | | | | V | | S |
| 51.7% | | | | | Q | | | Q | | | | | | | L | | |
| | | | | | | | | G | | | | | | | | | |

| | Modification set No. | | | | | | | | | | | | | | |
|---|---|---|---|---|---|---|---|---|---|---|---|---|---|---|---|
| | 5 | | | | 6 | | | | 7 | | | 8 | | | |
| | EU numbering | | | | | | | | | | | | | | |
| | 356 | 399 | 409 | 439 | 356 | 392 | 399 | 409 | 392 | 399 | 409 | 356 | 392 | 399 | 439 |
| Combinations | K | K | E | E | K | D | K | D | D | K | D | K | D | K | E |
| of | | | | | | | | | | | | | | | |
| modifications | | | | | | | | | | | | | | | |
| that showed | | | | | | | | | | | | | | | |
| strong | | | | | | | | | | | | | | | |
| suppression | | | | | | | | | | | | | | | |
| of | | | | | | | | | | | | | | | |
| heteromeric | | | | | | | | | | | | | | | |
| association in | | | | | | | | | | | | | | | |
| Table 1 | | | | | | | | | | | | | | | |
| Amino acid | R | R | D | D | R | E | R | E | E | R | E | R | E | R | D |
| modifications | H | H | | | H | | H | | | | | | | | |
| in Table 15 | | | | | | | | | | | | | | | |
| that | | | | | | | | | | | | | | | |
| suppressed | | | | | | | | | | | | | | | |
| the hetero- | | | | | | | | | | | | | | | |
| multimer | | | | | | | | | | | | | | | |
| ratio below | | | | | | | | | | | | | | | |
| that of WT, | | | | | | | | | | | | | | | |
| 51.7% | | | | | | | | | | | | | | | |

TABLE 20
Combinations of modifications that showed strong suppression of heteromeric association in Table 1, and amino acid modifications
at the respective residue positions that suppressed the heteromultimer ratio to 10% or lower in Table 15

| | Modification set No. | | | | | | | | | | | | | | | | |
|---|---|---|---|---|---|---|---|---|---|---|---|---|---|---|---|---|---|
| | 1 | | 2 | | | 3 | | | | | 4 | | | | | | |
| | EU numbering | | | | | | | | | | | | | | | | |
| | 394 | 405 | 366 | 368 | 407 | 347 | 360 | 399 | 405 | 409 | 345 | 347 | 360 | 366 | 399 | 407 | 409 |
| Combinations | W | A | W | A | V | R | E | V | T | W | R | R | D | V | M | A | V |
| of | | | | | | | | | | | | | | | | | |
| modifications | | | | | | | | | | | | | | | | | |
| that showed | | | | | | | | | | | | | | | | | |
| strong | | | | | | | | | | | | | | | | | |
| suppression of | | | | | | | | | | | | | | | | | |
| heteromeric | | | | | | | | | | | | | | | | | |
| association in | | | | | | | | | | | | | | | | | |
| Table 1 | | | | | | | | | | | | | | | | | |
| Amino acid | F | S | Y | S | L | K | D | L | A | F | E | K | E | | Q | | Q |
| modifications | Y | T | F | T | I | Y | | I | V | Y | | | | | N | | N |
| in Table 15 | | C | | C | M | H | | M | S | H | | | | | H | | H |
| that suppressed | | G | | V | A | | | S | C | | | | | | I | | L |
| the hetero- | | V | | G | S | | | T | N | | | | | | F | | I |
| multimer ratio | | | | | T | | | C | D | | | | | | Y | | F |
| to 10% or | | | | | C | | | H | G | | | | | | T | | Y |
| lower | | | | | N | | | A | | | | | | | S | | T |
| | | | | | Q | | | N | | | | | | | V | | S |
| | | | | | | | | Q | | | | | | | L | | |
| | | | | | | | | G | | | | | | | | | |

| | Modification set No. | | | | | | | | | | | | | | |
|---|---|---|---|---|---|---|---|---|---|---|---|---|---|---|---|
| | 5 | | | | 6 | | | | 7 | | | 8 | | | |
| | EU numbering | | | | | | | | | | | | | | |
| | 356 | 399 | 409 | 439 | 356 | 392 | 399 | 409 | 392 | 399 | 409 | 356 | 392 | 399 | 439 |
| Combinations | K | K | E | E | K | D | K | D | D | K | D | K | D | K | E |
| of | | | | | | | | | | | | | | | |
| modifications | | | | | | | | | | | | | | | |
| that showed | | | | | | | | | | | | | | | |
| strong | | | | | | | | | | | | | | | |
| suppression | | | | | | | | | | | | | | | |
| of | | | | | | | | | | | | | | | |
| heteromeric | | | | | | | | | | | | | | | |
| association in | | | | | | | | | | | | | | | |
| Table 1 | | | | | | | | | | | | | | | |
| Amino acid | R | R | D | D | R | E | R | E | E | R | E | R | E | R | D |
| modifications | H | H | | | H | | H | | | | | | | | |
| in Table 15 | | | | | | | | | | | | | | | |
| that | | | | | | | | | | | | | | | |
| suppressed | | | | | | | | | | | | | | | |
| the hetero- | | | | | | | | | | | | | | | |
| multimer | | | | | | | | | | | | | | | |
| ratio to 10% | | | | | | | | | | | | | | | |
| or lower | | | | | | | | | | | | | | | |

TABLE 21
Combinations of modifications that showed strong suppression of heteromeric association in Table 1, and amino acid
modifications at the respective residue positions that suppressed the heteromultimer ratio to 0% in Table 15

| | Modification set No. | | | | | | | | | | | | | | | | |
|---|---|---|---|---|---|---|---|---|---|---|---|---|---|---|---|---|---|
| | 1 | | 2 | | | 3 | | | | | 4 | | | | | | |
| | EU numbering | | | | | | | | | | | | | | | | |
| | 394 | 405 | 366 | 368 | 407 | 347 | 360 | 399 | 405 | 409 | 345 | 347 | 360 | 366 | 399 | 407 | 409 |
| Combinations | W | A | W | A | V | R | E | V | T | W | R | R | D | V | M | A | V |
| of | | | | | | | | | | | | | | | | | |
| modifications | | | | | | | | | | | | | | | | | |
| that showed | | | | | | | | | | | | | | | | | |
| strong | | | | | | | | | | | | | | | | | |
| suppression of | | | | | | | | | | | | | | | | | |
| heteromeric | | | | | | | | | | | | | | | | | |
| association in | | | | | | | | | | | | | | | | | |
| Table 1 | | | | | | | | | | | | | | | | | |
| Amino acid | F | S | Y | C | L | K | D | L | A | F | E | K | E | | N | | Q |
| modifications | | T | F | G | C | Y | | I | V | Y | | | | | H | | H |
| in Table 15 | | G | | T | A | H | | M | | | | | | | I | | L |
| that | | | | V | M | | | S | | | | | | | F | | I |
| suppressed the | | | | | I | | | T | | | | | | | Y | | F |
| hetero- | | | | | | | | C | | | | | | | T | | Y |
| multimer ratio | | | | | | | | H | | | | | | | S | | T |
| to 0% | | | | | | | | N | | | | | | | V | | S |
| | | | | | | | | G | | | | | | | L | | |
| | | | | | | | | A | | | | | | | Q | | |

| | Modification set No. | | | | | | | | | | | | | | |
|---|---|---|---|---|---|---|---|---|---|---|---|---|---|---|---|
| | 5 | | | | | 6 | | | 7 | | | 8 | | | |
| | EU numbering | | | | | | | | | | | | | | |
| | 356 | 399 | 409 | 439 | 356 | 392 | 399 | 409 | 392 | 399 | 409 | 356 | 392 | 399 | 439 |
| Combinations | K | K | E | E | K | D | K | D | D | K | D | K | D | K | E |
| of | | | | | | | | | | | | | | | |
| modifications | | | | | | | | | | | | | | | |
| that showed | | | | | | | | | | | | | | | |
| strong | | | | | | | | | | | | | | | |
| suppression | | | | | | | | | | | | | | | |
| of | | | | | | | | | | | | | | | |
| heteromeric | | | | | | | | | | | | | | | |
| association in | | | | | | | | | | | | | | | |
| Table 1 | | | | | | | | | | | | | | | |
| Amino acid | R | R | D | D | R | E | R | E | | | E | | | | |
| modifications | | H | | | H | | H | | | | | | | | |
| in Table 15 | | | | | | | | | | | | | | | |
| that | | | | | | | | | | | | | | | |
| suppressed | | | | | | | | | | | | | | | |
| the hetero- | | | | | | | | | | | | | | | |
| multimer | | | | | | | | | | | | | | | |
| ratio to 0% | | | | | | | | | | | | | | | |

### [Example 13] Evaluation of Coexpression of Homodimer and Heteromultimer

The amino acid modifications discovered in the present invention which promote homodimer formation may be able to be used concurrently with amino acid modifications promoting heterodimer formation (e.g., knob-into-hole modifications) To verify this possibility, antibodies containing some of the modifications that showed strong homodimer formation ability in IgG and knob-into-hole modifications were coexpressed, and the efficiencies (%) of formation of the homodimer and heterodimer of interest when coexpressed with WT-IgG1 were compared by IEC analysis. Antibody preparation and IEC analysis were performed according to the methods described in Example 6. Samples were detected at an excitation wavelength of 280 nm and a fluorescence wavelength of 330 nm (denoted as EU on the vertical axes in figures).

The correspondences between the amino acid modifications promoting heavy chain homodimer formation and heterodimer formation, SEQ ID NOs, and area ratios are shown in Table 22 (the non-introduction of modifications is denoted as “−”). This test was performed using the template antibody pairs of two homodimers and two heteromultimers (In Table 22, the template antibody group “hetero1+homo1” refers to the pair of a heterodimer containing SEQ ID NOs: 561 and 562 and a homodimer containing SEQ ID NO: 563, and the template antibody group “hetero2+homo2” refers to the pair of a heterodimer containing SEQ ID NOs: 567 and 568 and a homodimer containing SEQ ID NO: 78. The respective homodimer formation-promoting modifications were introduced into the homodimers.) The chromatograms of each antibody analysis are shown in FIGS. 6-1 to 6-10. The elution positions of the intended heavy chain heterodimer and heavy chain homodimer, and the area ratio of unintended multimers were calculated by assignment of peaks based on the elution positions of the respective reference samples.

This result showed that the amino acid modifications discovered in the present invention which promote homodimer formation can be used concurrently with amino acid modifications promoting heteromultimers (e.g., heterodimer formation). It was also shown that this effect allows more efficient expression of the homodimer and heteromultimer of interest than expression of the homodimer of WT-IgG.

TABLE 22
Combinations of modifications promoting heavy chain heterodimer formation and modifications
promoting heavy chain homodimer formation, and the reference samples of unintended multimers

| | | | | Modifications | | | Modifications | | | Modifications | Light |
|---|---|---|---|---|---|---|---|---|---|---|---|
| | | Heavy | Heavy | that promote | Heavy | Heavy | that promote | Heavy | Heavy | that promote | chain: |
| Template | | chain 1: | chain | formation of | chain 2: | chain | formation of | chain 3: | chain | formation of | SEQ |
| Antibody | Sample | SEQ ID | abbre- | heavy chain | SEQ ID | abbre- | heavy chain | SEQ ID | abbre- | heavy chain | ID |
| Group | No. | NO | viation | heterodimers | NO | viation | heterodimers | NO | viation | homodimers | NO |
| Hetero1 + | 1151 | 561 | K | T366W | | | | | | | 61 |
| Homo1 | 1152 | 562 | H | T366S/L368A/ | | | | | | | 61 |
| | | | | Y407V | | | | | | | |
| | 1153 | 561 | K | T366W | 562 | H | T366S/L368A/ | | | | 61 |
| | | | | | | | Y407V | | | | |
| | 1154 | | | | | | | 563 | e | — | 61 |
| | 1155 | 561 | K | T366W | | | | 563 | e | — | 61 |
| | 1156 | 562 | H | T366S/L368A/ | | | | 563 | e | — | 61 |
| | | | | Y407V | | | | | | | |
| | 1157 | 561 | K | T366W | 562 | H | T366S/L368A/ | 563 | e | — | 61 |
| | | | | | | | Y407V | | | | |
| | 1158 | | | | | | | 564 | E | T394W/F405A | 61 |
| | 1159 | 561 | K | T366W | | | | 564 | E | T394W/F405A | 61 |
| | 1160 | 562 | H | T366S/L368A/ | | | | 564 | E | T394W/F405A | 61 |
| | | | | Y407V | | | | | | | |
| | 1161 | 561 | K | T366W | 562 | H | T366S/L368A/ | 564 | E | T394W/F405A | 61 |
| | | | | | | | Y407V | | | | |
| | 1162 | | | | | | | 565 | E′ | E356K/K392D/ | 61 |
| | | | | | | | | | | D399K/K439E | |
| | 1163 | 561 | K | T366W | | | | 565 | E′ | E356K/K392D/ | 61 |
| | | | | | | | | | | D399K/K439E | |
| | 1164 | 562 | H | T366S/L368A/ | | | | 565 | E′ | E356K/K392D/ | 61 |
| | | | | Y407V | | | | | | D399K/K439E | |
| | 1165 | 561 | K | T366W | 562 | H | T366S/L368A/ | 565 | E′ | E356K/K392D/ | 61 |
| | | | | | | | Y407V | | | D399K/K439E | |
| | 1166 | | | | | | | 82 | E″ | Q347R/K360E/ | 61 |
| | | | | | | | | | | D399V/F405T/ | |
| | | | | | | | | | | K409W | |
| | 1167 | 561 | K | T366W | | | | 82 | E″ | Q347R/K360E/ | 61 |
| | | | | | | | | | | D399V/F405T/ | |
| | | | | | | | | | | K409W | |
| | 1168 | 562 | H | T366S/L368A/ | | | | 82 | E″ | Q347R/K360E/ | 61 |
| | | | | Y407V | | | | | | D399V/F405T/ | |
| | | | | | | | | | | K409W | |
| | 1169 | 561 | K | T366W | 562 | H | T366S/L368A/ | 82 | E″ | Q347R/K360E/ | 61 |
| | | | | | | | Y407V | | | D399V/F405T/ | |
| | | | | | | | | | | K409W | |
| Hetero2 + | 1170 | 567 | K′ | Y349C/T366W | | | | | | | 61 |
| Homo2 | 1171 | 568 | H′ | E356C/T366S/ | | | | | | | 61 |
| | | | | L368A/Y407V | | | | | | | |
| | 1172 | 567 | K′ | Y349C/T366W | 568 | H′ | E356C/T366S/ | | | | 61 |
| | | | | | | | L368A/Y407V | | | | |
| | 1173 | | | | | | | 78 | f | — | 61 |
| | 1174 | 567 | K′ | Y349C/T366W | | | | 78 | f | — | 61 |
| | 1175 | 568 | H′ | E356C/T366S/ | | | | 78 | f | — | 61 |
| | | | | L368A/Y407V | | | | | | | |
| | 1176 | 567 | K′ | Y349C/T366W | 568 | H′ | E356C/T366S/ | 78 | f | — | 61 |
| | | | | | | | L368A/Y407V | | | | |
| | 1177 | | | | | | | 570 | F | T394W/F405A | 61 |
| | 1178 | 567 | K′ | Y349C/T366W | | | | 570 | F | T394W/F405A | 61 |
| | 1179 | 568 | H′ | E356C/T366S/ | | | | 570 | F | T394W/F405A | 61 |
| | | | | L368A/Y407V | | | | | | | |
| | 1180 | 567 | K′ | Y349C/T366W | 568 | H′ | E356C/T366S/ | 570 | F | T394W/F405A | 61 |
| | | | | | | | L368A/Y407V | | | | |
| | 1181 | | | | | | | 571 | F′ | E356K/K392D/ | 61 |
| | | | | | | | | | | D399K/K439E | |
| | 1182 | 567 | K′ | Y349C/T366W | | | | 571 | F′ | E356K/K392D/ | 61 |
| | | | | | | | | | | D399K/K439E | |
| | 1183 | 568 | H′ | E356C/T366S/ | | | | 571 | F′ | E356K/K392D/ | 61 |
| | | | | L368A/Y407V | | | | | | D399K/K439E | |
| | 1184 | 567 | K′ | Y349C/T366W | 568 | H′ | E356C/T366S/ | 571 | F′ | E356K/K392D/ | 61 |
| | | | | | | | L368A/Y407V | | | D399K/K439E | |
| | 1185 | | | | | | | 572 | F″ | Q347R/K360E/ | 61 |
| | | | | | | | | | | D399V/F405T/ | |
| | | | | | | | | | | K409W | |
| | 1186 | 567 | K′ | Y349C/T366W | | | | 572 | F″ | Q347R/K360E/ | 61 |
| | | | | | | | | | | D399V/F405T/ | |
| | | | | | | | | | | K409W | |
| | 1187 | 568 | H′ | E356C/T366S/ | | | | 572 | F″ | Q347R/K360E/ | 61 |
| | | | | L368A/Y407V | | | | | | D399V/F405T/ | |
| | | | | | | | | | | K409W | |
| | 1188 | 567 | K′ | Y349C/T366W | 568 | H′ | E356C/T366S/ | 572 | F″ | Q347R/K360E/ | 61 |
| | | | | | | | L368A/Y407V | | | D399V/F405T/ | |
| | | | | | | | | | | K409W | |

The amino acid modifications contained in the heavy chain, which are denoted as heavy chain abbreviations in the table, are as follows:
K T366W
H T366S/L368A/Y407V
K′ Y349C/T366W
H′ E356C/T366S/L368A/Y407V
e −
T394W/F405A
E′ E356K/K392D/D399K/K439E
E″ Q347R/K360E/D399V/F405T/K409W
f −
F T394W/F405A
F′ E356K/K392D/D399K/K439E
F″ Q347R/K360E/D399V/F405T/K409W

In this test, two types of knob-into-hole modifications were used as heteromultimer-promoting modifications; however, other reported modifications may also be used. For example, modifications that may be used include strand-exchange engineered domain (SEED) body, which utilizes the differences in association preference between Ig classes, heterodimeric T-cell receptor (TCR) α and β chains (BEAT Fc), T366W/S354C-T366S/L368A/Y407V/Y349C, K409D/K392D-D399K/E356K, S364H/F405A-Y349T/T394F, D221E/P228E/L368E-D221R/P228R/K409R, F405L-K409R, T350V/T366L/K392L/T394W-T350V/L351Y/F405A/Y407V, K360E/K409W-Q347R/D399V/F405T, K360E/K409W/Y349C-Q347R/D399V/F405T/S354C, K370E/K409W-E357N/D399V/F405T, K360D/D399M/Y407A-E345R/Q347R/T366V/K409V, Y349S/K370Y/T366M/K409V-E356G/E357D/S364Q/Y407A, L351D/L368E-L351K/T366K, and L368D/K370S-E357Q/S364K (separation between two different heavy chains is denoted as “−”). In this case, as modifications for promoting homodimer formation, it is more preferable to use, e.g., a set of modifications that do not have modifications at the same residue numbers as, or that are largely different in the properties of amino acid (in terms of size or the presence/absence of charge) from, the amino acid modifications for promoting heteromeric association because this is expected to be less likely to result in promotion of unintended association due to both types of modifications.

In this test, one heteromultimer and one homomultimer were expressed; however, the number of samples to be coexpressed is not limited thereto. For example, when multiple heteromultimers are expressed, modifications at residue numbers different from each other are preferentially combined. It is further possible to express multiple homomultimers having amino acid modifications for promoting homomeric association that are different in amino acid residue number from those modifications for promoting association of the multiple heteromultimers, or that contain modifications at the same residue numbers but composed of amino acids with different properties. This will be able to allow multiple heteromultimers and multiple homomultimers to be expressed simultaneously with high purity.

### [Example 14] Evaluation of Heavy-Chain Homodimer Formation-Promoting Ability in Mammalian Expression System Using mRNA

Next, it was examined whether the transfection of mammalian cells with messenger RNAs (mRNAs) rather then plasmids would also yield the effect of promoting homodimer formation. The antibodies set forth in Table 25 were transiently expressed in mammalian cells by a method known to those skilled in the art using mRNAs containing sequences encoding the antibody heavy and light chains set forth in Tables 23 and 24, and purified by a method known to those skilled in the art. Specifically, plasmid DNAs containing sequences encoding the antibody heavy and light chains set forth in Tables 23 and 24 were used as templates and subjected to PCR to add a T7 promoter sequence and a poly A sequence. The constructed PCR fragments were used as templates to prepare mRNAs by in vitro transcription (IVT) method using MEGAscript T7 Transcription Kit (Thermo Fisher Scientific, AM1333). In transcription, ARCA (TriLink BioTechnologies, N-7003-1) was added as a cap structure, and 1-methylpseudouridine-5′-triphosphate (Trilink BioTechnologies, N-1081-1) as a modified nucleic acid. The prepared mRNAs were transfected into 1 mL of 2.5E+6 cells/mL Expi293 cells at the mass ratios set forth in Table 25 to express the antibodies transiently. After 4 days of transfection, the culture supernatant was collected and the antibodies were purified by a method known to those skilled in the art.

The prepared antibodies were analyzed by the IEC method described in Example 6. The respective area ratios resulting from the three-chain transfection are shown in Table 25. The constructs containing amino acid modifications for promoting homodimer formation showed a marked decrease in the ratio of heteromultimers.

The above results confirm that the amino acid modifications for promoting homodimer formation are effective even when mRNAs are used to express antibodies in mammalian cells.

TABLE 23
mRNAs containing sequences encoding antibody heavy chains

| | | | | Modifications that | | | | ORF |
|---|---|---|---|---|---|---|---|---|
| | | | | promote formation | mRNA ID | mRNA ID | Amino acid | nucleotide |
| Antibody heavy | | Heavy chain | IgG | of heavy chain | (Example | (Examples | sequence: | sequence: |
| chain name | Antigen | abbreviation | subclass | homodimers | 14) | 15, 16) | SEQ ID NO | SEQ ID NO |
| TR01H113-G1T6 | CD3 | T | IgG1 | — | TW_EBL_A1 | WOTL93145 | 573 | 611 |
| GCH065-G1T6 | GPC3 | G | IgG1 | — | TW_EBL_B1 | WOTL93146 | 607 | 612 |
| TR01H113-G1T6v1 | CD3 | T1 | IgG1 | Q347R/K360E/ | TW_EBL_C1 | WOTL93147 | 574 | 613 |
| | | | | D399V/F405T/ | | | | |
| | | | | K409W | | | | |
| GCH065-G1T6v2 | GPC3 | G2 | IgG1 | E356K/K392D/ | TW_EBL_D1 | WOTL93148 | 608 | 614 |
| | | | | D399K/K439E | | | | |
| Q499-G4d | FIXa | Q | IgG4 | — | TW_EBL_E1 | WOTL93149 | 575 | 615 |
| J327-G4d | FX | J | IgG4 | — | TW_EBL_F1 | WOTL93150 | 609 | 616 |
| Q499-G4dv3 | FIXa | Q3 | IgG4 | T394W/F405A | TW_EBL_G1 | WOTL93151 | 576 | 617 |
| J327-G4dv4 | FX | J4 | IgG4 | T366W/L368A/ | TW_EBL_H1 | WOTL93152 | 610 | 618 |
| | | | | Y407V | | | | |

TABLE 24
mRNAs containing sequences encoding antibody light chains

| Antibody light | | mRNA ID | mRNA ID | Amino acid sequence: | ORF nucleotide sequence: |
|---|---|---|---|---|---|
| chain name | Antigen | (Example 14) | (Examples 15, 16) | SEQ ID NO | SEQ ID NO |
| L0011-kT0 | CD3, GPC3 | TW_EBL_A2 | WOTL93153 | 577 | 619 |
| L404-kT0 | FIXa, FX | TW_EBL_B2 | WOTL93154 | 578 | 620 |

TABLE 25
Correspondences of mRNAs used in in vitro test and CIEX analysis results

| | | | | | CIEX analysis of samples expressed | | |
|---|---|---|---|---|---|---|---|
| | | | | | in vitro [Example 14] | | |
| | | | | | Heavy | Heavy | Heavy |
| | | | | | chain | chain | chain |
| | | | | mRNA mass | homo- | hetero- | homo- |
| | | | | ratio | multimer | multimer | multimer |
| | mRNA1 | mRNA2 | mRNA3 | (mRNA1/ | (GG, G2G2, | (TG, T1G2, | (TT, T1T1, |
| | (Heavy | (Light | (Heavy | mRNA2/ | JJ, J4J4) | QJ, Q3J4) | QQ, Q3Q3) |
| Antibody name | chain 1) | chain) | chain 2) | mRNA3) | (%) | (%) | (%) |
| TR01H113-G1T6/L0011- | TW_EBL_A1 | TW_EBL_A2 | TW_EBL_B1 | 1/4/1 | 24.59 | 53.68 | 21.73 |
| kT0//GCH065- | | | | | | | |
| G1T6/L0011-kT0 | | | | | | | |
| TR01H113-G1T6/L0011- | TW_EBL_A1 | TW_EBL_A2 | TW_EBL_D1 | 1/4/1 | 47.1 | 2.07 | 50.84 |
| kT0//GCH065- | | | | | | | |
| G1T6v2/L0011-kT0 | | | | | | | |
| TR01H113- | TW_EBL_C1 | TW_EBL_A2 | TW_EBL_B1 | 1/4/1 | 55.03 | 0 | 44.97 |
| G1T6v1/L0011-kT0//GCH065- | | | | | | | |
| G1T6/L0011-kT0 | | | | | | | |
| TR01H113- | TW_EBL_C1 | TW_EBL_A2 | TW_EBL_D1 | 1/4/1 | 46.13 | 0 | 53.87 |
| G1T6v1/L0011-kT0//GCH065- | | | | | | | |
| G1T6v2/L0011-kT0 | | | | | | | |
| Q499-G4d/L404- | TW_EBL_E1 | TW_EBL_B2 | TW_EBL_F1 | 1/4/1 | 13.64 | 48.9 | 37.46 |
| kT0//J327-G4d/L404-kT0 | | | | | | | |
| Q499-G4d/L404- | TW_EBL_E1 | TW_EBL_B2 | TW_EBL_H1 | 1/4/1 | 27.71 | 0 | 72.29 |
| kT0//J327-G4dv4/L404-kT0 | | | | | | | |
| Q499-G4dv3/L404- | TW_EBL_G1 | TW_EBL_B2 | TW_EBL_F1 | 1/4/1 | 39.96 | 0 | 60.04 |
| kT0//J327-G4d/L404-kT0 | | | | | | | |
| Q499-G4dv3/L404- | TW_EBL_G1 | TW_EBL_B2 | TW_EBL_H1 | 1/4/1 | 27.47 | 0 | 72.53 |
| kT0//J327-G4dv4/L404-kT0 | | | | | | | |

### [Example 15] Preparation of mRNA-Encapsulated Lipid Nanoparticles (mRNA-LNPs)

Lipid nanoparticles encapsulating the mRNAs shown in Tables 23 and 24 (mRNA-LNPs) were prepared using the general lipid composition and preparation method described in references etc. (Hou X, et al., Nat Rev Mater. 2021; 6(12):1078-1094, Weng Y, et al., Biotechnol Adv. 2020 May-June; 40:107534, Webb C, et al., Mol Pharm. 2022 Apr. 4; 19 (4): 1047-1058). Specifically, cationic lipid, DOPE, cholesterol, and PEG lipid were dissolved in EtOH at a molar ratio of 35:16:46.5:1.5 and at a total lipid concentration of about 1.3 mg/mL. The untranslated region (UTR) of mRNA was designed and the full-length mRNA was synthesized by Trilink, using 5-methoxyuridine as a modified nucleic acid and CleanCap AG as a cap structure. The prepared mRNAs were diluted to about 0.02 mg/mL in 25 mM sodium acetate buffer (pH 4.0). mRNA-LNPs were prepared by mixing the lipid ethanol solution and the aqueous mRNA solution at a ratio of 1:3 (vol/vol) using NanoAssemblr Ignite (Precision NanoSystems). The prepared mRNA-LNPs were diluted with about three times their volume of PBS. Then, the external buffer components were replaced with PBS using Amicon 100 kDa centrifugal filter (Millipore), and the mRNA-LNPs were concentrated to a desired concentration. The resulting mRNA-LNPs were filtered using a 0.22 μm sterile filter. The final mRNA-LNPs were stored at −80° C. until further use.

The particle diameter, polydispersity index (PdI), and zeta potential of mRNA-LNPs were measured using Zetasizer NanoZS (Malvern Panalytical). The mRNA-LNP samples were diluted 20 times in PBS before measuring the particle diameter and PdI, or diluted 170 times in 10 mM phosphate buffer (pH7.2) before measuring the zeta potential.

The total RNA concentration and free RNA in the mRNA-LNP samples were determined by a fluorescence-based assay (Ribogreen® reagent, Thermo Fisher Scientific). Encapsulation efficiency is calculated as (total RNA—free RNA)/total RNA. The mRNA-LNP samples were appropriately diluted with 1×TBE buffer containing 1% Triton-X100 and Ribogreen® reagent for determination of total RNA, or diluted with 1×TBE buffer containing only Ribogreen® reagent for determination of free RNA. A calibration curve was produced using the starting RNA solution used for preparing mRNA-LNP and diluted with 1×TBE buffer containing Ribogreen® reagent+/−1.0% Triton-X100. Samples containing a mixture of Triton-X100, Ribogreen® reagent, buffer, and mRNA-LNP sample were incubated at room temperature for about 5 minutes in the absence of light, and measured using SpectraMax M3 Microplate Reader (Molecular Devices) with the excitation, auto cutoff, and emission wavelengths set to 488 nm, 515 nm, and 525 nm, respectively. Total RNA and free RNA were determined from an appropriate calibration curve.

When LNPs were prepared in a typical manner, the encapsulation, particle size, and PdI were >90%, <100 nm, and <0.1, respectively.

LNPs encapsulating the corresponding mRNAs were mixed to give the combinations of mRNAs 1-3 shown in Table 26, and subjected to the in vivo test of Example 16.

TABLE 26
Names of mRNA-LNPs used in the in vivo test and mRNAs contained, and abbreviations of in vivo expression samples

| | | | | mRNA mass | Abbreviation of |
|---|---|---|---|---|---|
| | | | | ratio | samples |
| | mRNA1 | mRNA2 | mRNA3 | (mRNA1/ | expressed in |
| | (Heavy | (Light | (Heavy | mRNA2/ | in vivo test |
| Antibody name | chain 1) | chain) | chain 2) | mRNA3) | [Example 16] |
| TR01H113-G1T6/L0011-kT0//GCH065-G1T6/L0011-kT0 | WOTL93145 | WOTL93153 | WOTL93146 | 1/4/1 | CD3-WT x GPC3-WT |
| TR01H113-G1T6/L0011-kT0//GCH065-G1T6v2/L0011-kT0 | WOTL93145 | WOTL93153 | WOTL93148 | 1/4/1 | CD3-WT x GPC3-Design2 |
| TR01H113-G1T6v1/L0011-kT0//GCH065-G1T6/L0011-KT0 | WOTL93147 | WOTL93153 | WOTL93146 | 1/4/1 | CD3-Design1 x |
| | | | | | GPC3-WT |
| TR01H113-G1T6v1/L0011-kT0//GCH065-G1T6v2/L0011-kT0 | WOTL93147 | WOTL93153 | WOTL93148 | 1/4/1 | CD3-Design1 x |
| | | | | | GPC3-Design2 |
| Q499-G4d/L404-kT0//J327-G4d/L404-kT0 | WOTL93149 | WOTL93154 | WOTL93150 | 1/4/1 | FIXa-WT x FX-WT |
| Q499-G4d/L404-kT0//J327-G4dv4/L404-kT0 | WOTL93149 | WOTL93154 | WOTL93152 | 1/4/1 | FIXa-WT x FX-Design4 |
| Q499-G4dv3/L404-kT0//J327-G4d/L404-kT0 | WOTL93151 | WOTL93154 | WOTL93150 | 1/4/1 | FIXa-Design3 x FX-WT |
| Q499-G4dv3/L404-kT0//J327-G4dv4/L404-kT0 | WOTL93151 | WOTL93154 | WOTL93152 | 1/4/1 | FIXa-Design3 x |
| | | | | | FX-Design4 |

### [Example 16] Evaluation of Heavy-Chain Homodimer Formation-Promoting Ability in Mouse In Vivo Expression Using mRNA-LNPs

To test the in vivo effect of amino acid modifications for promoting heavy-chain homodimer formation, the mRNA-LNPs produced in Example 15 were intravenously administered to C57BL6/J mice (male, 6 weeks old) at 1.0 mg RNA/kg. The correspondences between the names of mRNA-LNPs used in the in vivo test, the mRNAs contained and their mass ratios, and the abbreviations of expression samples, are shown in Table 26.

Whole blood of mice was collected from the jugular vein after 3 and 7 days of administration. After centrifugation at 4° C., 12,000 rpm for 5 minutes, plasma was collected.

The homodimers and heterodimers in the collected plasma were measured. This measurement was carried out using Gyrolab xP workstation or Gyrolab xPand (Gyros Protein Technologies).

First, Human GPC3 Core Protein, TIA0124-rabbitFc (anti-idiotype (ID) antibody for anti-CD3 antibody (TR01H113-GIT6/L0011-KTO and TR01H113-GIT6v1/L0011-kTO)), rAQ8-mIgG2b described in WO2016047652 (anti-ID antibody for anti-FIXa antibody (Q499-G4d/L404-kTO and Q499-G4dv3/L404-KTO)), and rAJ540-rbtIgG (anti-ID antibody for anti-FX antibody (J327-G4d/L404-KTO and J327-G4dv4/L404-kT0)) were labeled with biotin and Alexa Fluor 647 using EZ-Link Sulfo-NHS-Biotin (21217, Thermo Fisher Scientific) and Alexa Fluor 647 Antibody Labeling Kit (A20186, Life Technologies) according to the manufacturers' protocols.

In the measurement of homodimer, the collected plasma or plasma diluted 2 to 200 times with pooled plasma of C57BL/6J (male, 8 weeks old, Jackson Laboratory Japan) was mixed with an equal volume of Rexxip A-max (P0004821, Gyros Protein Technologies). The plasma mixed with an equal volume of Rexxip A-max was mixed with the biotin-labeled antibody and Alexa Fluor 647-labeled antibody diluted with Rexxip A (P0004820, Gyros Protein Technologies) at 1:1:1 (the final concentration of the biotin-labeled antibody and the Alexa Fluor 647-labeled antibody was 1 μg/mL), and incubated at room temperature for 2 hours.

In the measurement of the anti-CD3 homodimer, a plasma sample was mixed with biotin-labeled TIA0124-rabbitFc and Alexa Fluor 647-labeled TIA0124-rabbitFc at 1:1:1. In the measurement of the anti-GPC3 homodimer, a plasma sample was mixed with biotin-labeled Human GPC3 Core Protein and Alexa Fluor 647-labeled Human GPC3 Core Protein at 1:1:1.

In the measurement of the anti-FIXa homodimer, a plasma sample was mixed with biotin-labeled rAQ8-mIgG2b and Alexa Fluor 647-labeled rAQ8-mIgG2b at 1:1:1.

In the measurement of the anti-FX homodimer, a plasma sample was mixed with biotin-labeled rAJ540-rbtIgG and Alexa Fluor 647-labeled rAJ540-rbtIgG at 1:1:1.

The incubated samples were measured using Gyrolab XP workstation or Gyrolab xPand. In this measurement, Gyrolab Bioaffy 1000 (P0004253, Gyros Protein Technologies) was used as a measurement disk. The above incubated mixture of the biotin-labeled antibody, Alexa Fluor 647-labeled antibody, and plasma sample was added onto the measurement disk, and the biotin-labeled antibody-homodimer-Alexa Fluor 647-labeled antibody complex was captured on the reaction layer in the disk. The amount of the captured complex was detected by the fluorescent signal of Alexa647 (Detect PMT 1%). The fluorescent signal of Alexa647 was analyzed with Gyro Evaluator (version 3.7.2.5976, Gyros Protein Technologies).

In the measurement of heterodimer, the collected plasma was diluted 3 to 5 times with pooled plasma of C57BL/6J (male, 8 weeks old, Jackson Laboratory Japan) and mixed with an equal volume of Rexxip A-max (P0004821, Gyros Protein Technologies).

The mixed plasma was measured using Gyrolab xP workstation or Gyrolab xPand. In this measurement, Gyrolab Bioaffy 1000 (P0004253, Gyros Protein Technologies) was used as a measurement disk. The biotin-labeled antibody diluted to 25 μg/mL with PBS 0.05% Tween 20 (P3563, SIGMA-ALDRICH) was added to the measurement disk and immobilized to the reaction layer in the disk. The above prepared plasma sample was added thereto, and the Alexa Flour 647-labeled antibody diluted to 5 μg/mL with Rexxip F (P0004825, Gyros Protein Technologies) was added. The captured heterodimer was detected with the fluorescent signal of Alexa647 (Detect PMT 1%). The fluorescent signal of Alexa647 was analyzed with Gyro Evaluator (version 3.7.2.5976, Gyros Protein Technologies).

In the measurement of the anti-CD3/GPC3 heterodimer, biotin-labeled Human GPC3 Core Protein was used as an immobilization antigen, and Alexa Fluor 647-labeled TIA0124-rabbitFc was used as a detection antibody.

In the measurement of the anti-FIXa/FX heterodimer, biotin-labeled rAQ8-mIgG2b was used as an immobilization antibody, and Alexa Fluor 647-labeled rAJ540-rbtIgG was used as a detection antibody.

As a result of this analysis, the plasma concentration of each antibody is shown in FIGS. 7-1 and 7-2 and FIGS. 7-3 and 7-4.

On day 3 after administration, for the WT antibodies which did not contain homomeric association-promoting modifications (CD3-WT×GPC3-WT and FIXa-WT×FX-WT), the expression of two types of homodimers and heterodimers was detected. For the Design antibodies which contained homomeric association-promoting modifications in either or both antibodies (CD3-WT×GPC3-Design2, CD3-Design1×GPC3-WT, CD3-Design1×GPC3-Design2, FIXa-WT×FX-Design4, FIXa-Design3×FX-WT, and FIXa-Design3×FX-Design4), the expression of homodimers was detected at a similar level as the WT antibodies or slightly increased. On the other hand, the heterodimer expression of the Design antibodies was significantly suppressed in all samples. Compared to the heterodimer expression in the WT antibodies, the expression of the anti-CD3/GPC3 heterodimer was suppressed by 72 to 878 times or more, and the expression of the anti-FIXa/FX heterodimer was suppressed by 241 to 446 times or more.

The suppression effect on the heterodimer expression continued even on day 7 after administration, showing 84- to 717-fold or more suppression of the expression of the anti-CD3/GPC3 heterodimer, and 184- to 375-fold or more suppression of the expression of the anti-FIXa/FX heterodimer.

This demonstrated that the Design antibodies can suppress the expression of heterodimers in vivo.

This test demonstrated the in vivo expression of two antibodies containing the Q347R/K360E/D399V/F405T/K409W or E356K/K392D/D399K/K439E modification in one or two heavy chains, and two antibodies containing the T394W/F405A or T366W/L368A/Y407V modification in one or two heavy chains. Similar suppression of heterodimer expression is also expected with the other amino acid modifications for which the promotion of homodimer formation was confirmed in the in vitro test using plasmids, in the other molecular formats and functional Fc, and in combinations of three or more antibodies.

Examples 1-16 above found a number of modifications that have a strong ability to promote heavy-chain homodimerization and also have superior physicochemical properties and Fc functions for therapeutic antibodies. Furthermore, an exhaustive study of combinations of modified Fc regions found a number of combinations that allow heavy-chain homodimerization to be maintained between modified Fc regions. Since these are antibodies that do not associate with WT-IgG, they make it possible to provide therapeutic drugs based on multiple antibodies that are homogeneous and safe when in vivo antibody expression is intended.

### INDUSTRIAL APPLICABILITY

The present disclosure provides polypeptides for which association between polypeptides is controlled, methods for producing polypeptides for which association is controlled, methods for controlling the association of polypeptides, nucleic acids encoding polypeptides for which association is controlled, compositions containing such a nucleic acid, and such. Because of the controlled association between polypeptides, the polypeptides of the present disclosure can particularly be used for production of antibodies and expression of therapeutic drugs in living organisms.

LENGTHY TABLES
The patent application contains a lengthy table section. A copy of the table is available in electronic form from the USPTO web site (https://seqdata.uspto.gov/docdetail?docId=US20260217835A1). An electronic copy of the table will also be available from the USPTO upon request and payment of the fee set forth in 37 CFR 1.19(b)(3).

1. A nucleic acid encoding a first polypeptide,
wherein the first polypeptide comprises an Fc region into which a modification has been introduced,
wherein, because of the modification introduced into the Fc region, the first polypeptide associates more readily with a first polypeptide having the modification via the Fc region than with a first polypeptide comprising an Fc region into which the modification has not been introduced.

2. The nucleic acid of claim 1, wherein the first polypeptide associates more readily with a first polypeptide having the modification via the Fc region than with a first polypeptide comprising an Fc region into which the modification has not been introduced due to at least one of the following actions resulting from the introduced modification: (1) steric complementarity, (2) disulfide linkage, and (3) electrostatic charge.

3. A composition comprising:
the nucleic acid of claim 1; and
a nucleic acid encoding a second polypeptide,
wherein the second polypeptide comprises an Fc region into which a modification has been introduced,
wherein, because of the modification introduced into the Fc region, the second polypeptide associates more readily with a second polypeptide having the modification via the Fc region than with a second polypeptide comprising an Fc region into which the modification has not been introduced.

4. A composition comprising:
the nucleic acid of claim 1;
a nucleic acid encoding a second polypeptide; and
a nucleic acid encoding a third polypeptide,
wherein the second polypeptide associates more readily with the third polypeptide than with the second polypeptide.

5. A composition comprising:
the nucleic acid of claim 2; and
a nucleic acid encoding a second polypeptide,
wherein the second polypeptide does not have the modification the first polypeptide has.

6. The composition of claim 3, wherein the modification introduced into the first polypeptide is different from the modification introduced into the second polypeptide.

7. The composition of claim 3, wherein the second polypeptide associates more readily with a second polypeptide having the modification via the Fc region than with a second polypeptide comprising an Fc region into which the modification has not been introduced due to at least one of the following actions resulting from the introduced modification: (1) steric complementarity, (2) disulfide linkage, and (3) electrostatic charge.

8. The nucleic acid of claim 1, wherein the modification introduced into the Fc region is at least one of the modifications set forth in Table 2.

9. The nucleic acid of claim 1, wherein the modification introduced into the Fc region is at least one of the modifications set forth in Table 4.

10. The nucleic acid of claim 6, wherein the modification introduced into the first polypeptide and the modification introduced into the second polypeptide are at least one of the combinations of modifications set forth in Table 6.

11. The nucleic acid of claim 1, wherein the polypeptide is an antibody.

12. The nucleic acid of claim 1, wherein the Fc region is an Fc region of IgG.

13. The nucleic acid of claim 1, wherein the polypeptide into which the modification has been introduced retains a function of the polypeptide prior to modification.

14. The nucleic acid of claim 12, wherein the Fc region into which the modification has been introduced retains a function of an Fc of IgG.

15. The nucleic acid of claim 1, wherein the Fc region is derived from any one of IgG1, 2, 3, and 4.

16. A host cell into which the nucleic acid of any one of claims 1, 2, and 8-14 or the composition of any one of claims 3-7 has been introduced.

17. A polypeptide expressed from the nucleic acid of claim 1.

18. A composition comprising a nucleic acid encoding a polypeptide,
wherein the polypeptide comprises an Fc region into which a modification has been introduced,
wherein the modification is at least one of the modifications set forth in Table 2.

19. A polypeptide which comprises an Fc region into which a modification has been introduced, wherein the polypeptide associates more readily with a polypeptide having the modification via the Fc region than with a polypeptide comprising an Fc region into which the modification has not been introduced due to at least one of the following actions resulting from the introduced modification: (1) steric complementarity, (2) disulfide linkage, and (3) electrostatic charge.

20. A method for obtaining a polypeptide for which association is controlled, comprising:
obtaining a nucleic acid encoding the polypeptide; and
expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

21. A method for controlling association of a homomer of a polypeptide, comprising:
obtaining a nucleic acid encoding the polypeptide; and
expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

22. A method for promoting expression of a homomer of a polypeptide, comprising:
obtaining a nucleic acid encoding the polypeptide; and
expressing the nucleic acid,
wherein the polypeptide comprises an Fc region, and
wherein, because of the modification introduced into the Fc region, the polypeptide associates more readily with a polypeptide having the modification via the Fc region than with a polypeptide comprising an Fc region into which the modification has not been introduced.

23. The composition of claim 4, wherein the second polypeptide comprises an Fc region into which a modification has been introduced,
wherein, because of the modification introduced into the Fc region, the second polypeptide associates more readily with the third polypeptide via the Fc region than with the second polypeptide.

24. The composition of claim 1, wherein the Fc region into which the modification has been introduced comprises an amino acid modification at one combination or two or more combinations of positions selected from the combinations of positions shown in (a) to (d) below according to EU numbering:
(a) positions 394 and 405;
(b) positions 366, 368, and 407;
(c) positions 347, 360, 399, 405 and 409; and
(d) positions 356, 392, 399, and 439.

25. The composition of claim 24, wherein the Fc region into which the modification has been introduced comprises at least one amino acid selected from the group consisting of:
(a) W, F, or Y at position 394, and
A, S, T, C, G, or V at position 405;
(b) W, Y, or F at position 366,
A, S, T, C, V, or G at position 368, and
V, L, I, M, A, S, T, C, N, or Q at position 407;
(c) R, K, Y, or H at position 347,
E or D at position 360,
V, L, I, M, S, T, C, H, A, N, Q, or G at position 399,
T, A, V, S, C, N, D, or G at position 405, and
W, F, Y, or H at position 409; and
(d) K or R at position 356,
D or E at position 392,
K or R at position 399, and
E or D at position 439,
according to EU numbering.

26. The composition of claim 24, wherein the Fc region into which the modification has been introduced comprises at least one amino acid selected from the group consisting of:
(a) W or F at position 394, and
A, S, T, or G at position 405;
(b) W, Y, or F at position 366,
A, T, C, V, or G at position 368, and
V, L, I, M, A, or C at position 407;
(c) R, K, Y, or H at position 347,
E or D at position 360,
V, L, I, M, S, T, C, H, A, N, or G at position 399,
T, A, or V at position 405, and
W, F, or Y at position 409; and
(d) K at position 356,
D at position 392,
K at position 399, and
E at position 439,
according to EU numbering.

27. The nucleic acid of claim 1, wherein the modification introduced into the Fc region is at least one of the modifications set forth in Table 15.

28. The nucleic acid of claim 1, wherein the modification introduced into the Fc region is at least one of the modifications set forth in Table 17.
